(19) United States
(12) Patent Application Publication
(10) Pub. No.: US 2003/0134398 A1
(43) Pub. Date: Jul. 17, 2003

(54) Mitomycin biosynthetic gene cluster

(76) Inventors: David H. Sherman, St. Louis Park, MN (US); Yingqing Mao, St. Paul, MN (US); Mustafa Varoglu, San Diego, CA (US); Min He, Monsey, NY (US); Paul Sheldon, St. Paul, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 09/953,348

(22) Filed: Sep. 12, 2001

(51) Int. Cl. C12P 17/16; C12P 17/18; C12N 9/06; C12N 9/10; C07H 21/04; C12P 21/02; C12N 1/21; C12N 15/74

(52) U.S. Cl. 435/119; 435/69.1; 435/193; 435/320.1; 435/252.3; 536/23.2; 435/191

(57) ABSTRACT

The invention provides a biosynthetic gene cluster for mitomycin, as well as methods of using gene(s) within the cluster to alter biosynthesis.

### BACKGROUND OF THE INVENTION

Streptomyces are filamentous Gram-positive soil bacteria with a nucleotide base composition greater than 70 mole % G+C (Stackebrandt and Woese, 1981). They produce a wide array of biologically active compounds including over two thirds of the commercially important natural product metabolites (Alderson et al., 1993; Bevax, 1998). Genetic information accumulated over the past 15 years has demonstrated that genes encoding enzymes for natural product assembly are clustered on the Streptomyces genome (Martin, 1992). In addition, one or more pathway-specific transcriptional regulatory genes, and at least one resistance gene are typically found within the antibiotic biosynthetic gene cluster (Chater, 1992). Heterologous hybridization with gene probes based on highly conserved biosynthetic enzyme amino acid sequences has been useful to clone antibiotic biosynthetic genes (Hopwood, 1997; Seno and Baltz, 1989; Turgay and Marahiel, 1994).

The mitomycins are a group of natural products that contain a variety of functional groups, including aminobenzoquinone and aziridine ring systems. One representative of the family, mitomycin C (MC), was the first recognized bioreductive alkylating agent. In particular, since its discovery and demonstration of anticancer activity in the 1960s, many aspects of the chemistry and biology of MC have been investigated. This has provided detailed information on its unprecedented molecular mechanism, unique biological and pharmacological properties, drug resistance, and bioactive analogues (Hata et al., 1956; Verweij, 1997). MC is regarded as the prototype natural product alkylating agent whose activity is dependent on the reductive activation (either chemically, such as low pH, or enzymatically, such as DT-diaphorase, NADH cytochrome c reductase) (Boxer, 1997; Cummings et al., 1998). Activated MC crosslinks double-stranded DNA, which in turn induces diverse biological effects including selective inhibition of DNA synthesis, mutagenesis, induction of DNA repair (SOS response), sister chromatid exchange, signal transduction, and induction of apoptosis (Tomasz and Palem, 1997). Tumor hypoxia and the increased expression of bioreductive enzymes in malignant cells create a selective environment for drug activation and make MC an attractive agent for anti-tumor therapy (Spanswick et al., 1998). MC has become one of the most effective antitumor drugs against non-small cell lung carcinoma and other soft tumors, as well as a clinically important component of combination cancer chemotherapy and radiotherapy of solid tumors (Henderson, 1993).

In addition to its biological and pharmacological importance, MC is prominent because its molecular mechanism represents a model for structurally related antitumor antibiotics such as porfiromycin (Pan and Iracki, 1988), mitiromycin (Wakaki et al., 1958), FR66979 (Paz and Hopkins, 1997), FR900482 (Williams et al., 1997), FK973 (Hirai et al., 1994), and FK317 (Naoe et al., 1998), as well as structurally unrelated bioreductive agents such as EO9 (Smitskampwilms et al., 1996), and tirapazamine (Evans et al., 1998). Numerous MC derivatives have been synthesized and tested for enhanced activities, including the recently identified selective protein tyrosine kinase inhibitor, la-docosahexaenoyl MC (Kasai and Arai, 1995; Shikano et al., 1998).

Streptomyces lavendulae produces MC. The molecule has an unusual structure comprised of aziridine, pyrrolizidine, pyrrolo-(1,2a)-indole, and amino-methylbenzoquinone rings to give the mitosane nucleus (Webb et al., 1962). The mitosane core of MC was shown to be derived from the junction of an arnino-methylbenzoquinone (mC₇N unit) and hexosamine (C₆N unit) (Homemann, 1981). The C₆N unit consists of carbons 1, 2, 3, 9, 9a, 10, with the aziridine nitrogen derived intact from D-glucosamine (Hornemann et al., 1974).

The mC₇N unit in MC and the ansamycins is derived from 3-amino-5-hydroxybenzoic acid (AHBA) (Becker et al., 1983; Kibby and Richards, 1981). AHBA was first shown to be incorporated into the ansamycin antibiotic actamycin (Kibby et al., 1980). Subsequently, it was confirmed as an efficient precursor for rifamycin (Becker et al., 1983; Kibby and Rickards, 1981; Ghilsalba and Neuesch, 1981), geldanamycin (Potgieter, 1983), ansamitocin (Hatano et al., 1982), ansatrienin (Wu et al., 1987), streptovaricin (Staley and Rinehart, 1991) and naphthomycin A (Lee et al., 1994). Anderson et al. (1980) demonstrated that [carboxy-¹³C] AHBA could be efficiently and specifically incorporated into the C-6 methyl group of porfiromycin, which contains the same mitosane core as MC. Incorporation experiments with radiolabeled precursors have demonstrated that the mitosane core of MC was derived from the junction of AHBA and D-glucosamine (Anderson et al., 1980; Hornemann, 1981).

Meanwhile the O- and N- (but not C-) methyl groups were shown to be derived from L-methionine, while the C-10 carbamoyl group came from L-arginine or L-citrulline (Bezanson and Vining, 1971; Hornemann and Eggert, 1975; Homemann et al., 1974). [¹⁴C]-labeled precursor feeding studies with D-glucose, pyruvate and D-erytrose indicated that de novo biosynthesis of AHBA resulted directly from the shildiate pathway. However, no incorporation into the mC₇N unit of either MC (Hornemann, 1981) or the ansamycin antibiotics (Chiao et al., 1998) was found from labeling studies with shikimic acid, the shikimate precursor 3-dehydroshikimic acid, or the shikdmate derived amino acids. These results led to the hypothesis of a modified shikimate pathway, in which a 3-deoxy-D-arabino-heptulosonic acid-7-phosphate (DAHP) synthase-like enzyme catalyzes the conversion to 3,4-dideoxy4-amino-D-arabino-heptulosonic acid-7-phosphate (amino-DABP), to give the ammoniated shikimate pathway (Kim et al., 1992). Floss (1997) provided strong support for this new variant of the shikimate pathway by showing that aminoDAHP, 5-deoxy-5-amino-3dehydroshikimic acid (aminoDHQ), and 5-deoxy-5-amino-3-dehydroshikimic acid (aminoDHS) could be efficiently converted into AHBA by a cell-free extract of Amycolatopsis mediteranei (rifamycin producer), in contrast to the normal shikimate pathway intermediate DAHP which was not converted (Kim et al., 1992; Kim et al., 1996). Recently, the AHBA synthase (rifK) gene from A. mediteranei has been cloned, sequenced and functionally characterized (Kim et al., 1998).

Little is known regarding the details of the convergent assembly of MC from AHBA and D-glucosamine in S. lavendulae, i.e., whether its de novo biosynthesis is related to the primary metabolic shikimate pathway, an important route in microorganisms and plants for aromatic amino acid biosynthesis (Floss, 1997). In addition, it is unclear how S. lavendulae resists the activity of MC since the preferred MC alkylation sites in DNA are guanine and cytosine, and MC-induced cell death can result from a single crosslink per genome (Tomasz, 1995).

Thus, there is a continuing need for the identification and isolation of antibiotic biosynthetic genes, including genes which confer resistance to antibiotics or result in enhanced production of antibiotics.

### SUMMARY OF THE INVENTION

The present invention provides an isolated and purified nucleic acid molecule, e.g., DNA, comprising a gene cluster for mitomycin, a variant or a fragment thereof (the mit/mmc gene cluster). As described hereinbelow, the S. lavendulae mitomycin gene cluster includes the mitomycin biosynthetic gene cluster comprising 47 mitomycin biosynthetic genes spanning 55 kb of contiguous DNA. The biosynthetic portion of the gene cluster includes genes that encode polypeptides involved in the generation of biosynthetic precursors, mitosane ring system assembly and functionalization (e.g., methylation, hydroxylation, aminotransfer, carbamoylation, and carbonyl reduction), a mitomycin resistance gene which is different than mrd and the unlinked mcr, as well as several regulatory genes. Gene disruption was employed to further characterize some of the genes. Fourteen of 22 gene disruption mutants affected mitomycin biosynthesis, resulting in abrogation or overexpression of drug production, e.g., targeted genetic disruption of a mitomycin pathway regulator (e.g., mmcW) led to a substantial increase in drug production. It is preferred that the isolated and purified nucleic acid molecule of the invention is nucleic acid from Streptomyces spp., such as Streptomyces lavendulae (e.g., B19/ATCC 27422, NRRL 2564, KY68 1, ATCC 27423, or PB 1000), Streptomyces caespitosus, Streptomyces verticillatus, and Streptomyces sandaensis (FERM-P7654), although isolated and purified nucleic acid molecules from other organisms which produce mitomycin or biological or functional equivalents thereof are also within the scope of the invention. The nucleic acid molecules of the invention are double-stranded or single-stranded.

As described hereinbelow, a 3.8 kb BamHI fragment from the S. lavendulae genome was isolated which comprises three open reading frames (ORFs). One of the ORFs (mitA) showed high similarity to previously identified AHBA synthase genes (Kim et al., 1998), while another (mitB) showed sequence similarity to several prokaryotic and eukaryotic glycosyltransferases. Nucleotide sequence analysis showed that mitA encodes a 388 amino acid protein that has 71% identity (80% similarity) with the rifamycin AHBA synthase from Amycolatopsis mediteranei, as well as with two additional AHBA synthases from related ansamycin antibiotic-producing microorganisms. Gene disruption and site-directed mutagenesis of the S. lavendulae chromosomal copy of mitA completely blocked the production of MC. The function of mitA was confirmed by complementation of a S. lavendulae strain containing a K191A mutation in MitA with 3-amino-5-hydroxybenzoic acid, i.e., MC production was restored when the mitA mutant strain was cultured in the presence of exogenous 3-amino-5-hydroxybenzoic acid. mitB encodes a 272 amino acid protein.

Seven gene products (aminoDHQ synthase (MitP), aminoquinate dehydrogenase (MitT), aminoDHQ dehydratase (MmcF), AHBA synthase (MitA), oxidoreductase (MitG), phosphatase (MitJ), and kinase (MitS)) are likely responsible for assembly of the intermediate 3-amino-5-hydroxybenzoic acid (AHBA) through a variant of the shikimate pathway. However, the gene encoding aminoDAHP synthase, the first presumed enzyme involved in AHBA biosynthesis from phosphoenol pyruvate (PEP) and erythrose 4-phosphate (E4P), is not linked within the mitomycin biosynthetic gene cluster.

A mitomycin resistance determinant (mct) encodes a membrane-associated protein involved in excretion of mitomycin from cells. Disruption of met by insertional inactivation resulted in a S. lavendulae mutant strain that was considerably more sensitive to MC. Expression of mct in E. coli conferred a 5-fold increase in cellular resistance to MC, led to the synthesis of a membrane associated protein, and correlated with reduced intracellular accumulation of drug. Co-expression of mct and mrd in E. coli resulted in a 150-fold increase in resistance, as well as reduced intracellular accumulation of MC. The results establish that MRD maintains a high affinity for MC and may serve as the primary receptor (participating as an accessory component in a drug export system) for subsequent transport by MCT.

The cloned mitomycin biosynthetic genes are useful to elucidate the molecular basis for the biosynthesis of the mitosane ring system, as well as to engineer the biosynthesis of novel natural products. Moreover, genetic engineering or overexpression of the transport, resistance and regulatory proteins may lead to higher titers of mitomycin compounds from production cultures.

Preferably, the isolated nucleic acid molecule comprising the gene cluster includes a nucleic acid sequence comprising SEQ ID NO:96 or SEQ ID NO:76, a variant or a fragment thereof, e.g., a nucleic acid molecule that hybridizes under moderate, or more preferably stringent, hybridization conditions to SEQ ID NO:96, SEQ ID NO:76, the complement thereof, or a fragment thereof. Moderate and stringent hybridization conditions are well known to the art, see, for example sections 9.47-9.51 of Sambrook et al. (Molecular Cloning: A Laboratory Manual, Cold Spring Harbor Laboratory, Cold Spring Harbor, N.Y. (1989). For example, stringent conditions are those that (1) employ low ionic strength and high temperature for washing, for example, 0.015 M NaCl/0.0015 M sodium citrate (SSC); 0.1% sodium lauryl sulfate (SDS) at 50° C., or (2) employ a denaturing agent such as formamide during hybridization, e.g., 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% sodium dodecylsulfate (SDS), and 10% dextran sulfate at 42° C., with washes at 42° C. in 0.2×SSC and 0.1% SDS.

A preferred nucleic molecule of the invention comprises a nucleic acid sequence encoding a polypeptide including, but not limited to, MitA (e.g., SEQ ID NO:10 encoded by SEQ ID NO:97), MitB (e.g., SEQ ID NO:11 encoded by SEQ ID NO:98), MitC (e.g., SEQ ID NO:12 encoded by SEQ ID NO:99), MitD (e.g., SEQ ID NO:100 encoded by SEQ ID NO:45), MitE (e.g., SEQ ID NO:101 encoded by SEQ ID NO:44), MitF (e.g., SEQ ID NO:102 encoded by SEQ ID NO:43), MitG (e.g., SEQ ID NO:103 encoded by SEQ ID NO:42), MitH (e.g., SEQ ID NO:104 encoded by SEQ ID NO:41), MitI (e.g., SEQ ID NO:105 encoded by SEQ ID NO:40), MitJ (e.g., SEQ ID NO:106 encoded by SEQ ID NO:39), MitK (e.g., SEQ ID NO:107 encoded by SEQ ID NO:38), MitL (e.g., SEQ ID NO:108 encoded by SEQ ID NO:37), MitM (e.g., SEQ ID NO:109 encoded by SEQ ID NO:36), MitN (e.g., SEQ ID NO:108 encoded by SEQ ID NO:35), MitO (e.g., SEQ ID NO:111 encoded by SEQ ID NO:34), MitP (e.g., SEQ ID NO:112 encoded by SEQ ID NO:33), MitQ (e.g., SEQ ID NO:113 encoded by SEQ ID NO:32), MitR (e.g., SEQ ID NO:114 encoded by SEQ ID NO:31), MitS (e.g., SEQ ID NO: 115 encoded by SEQ ID NO:30), MitT (e.g., SEQ ID NO:140 encoded by SEQ ID NO:29), MmcA (SEQ ID NO:116 encoded by SEQ ID NO:49), MmcB (SEQ ID NO:117 encoded by SEQ ID NO:50), MmcC (SEQ ID NO:118 encoded by SEQ ID NO:51), MmcD (SEQ ID NO:119 encoded by SEQ ID NO:52), MmcE (SEQ ID NO:120 encoded by SEQ ID NO:53), MmcF (SEQ ID NO:121 encoded by SEQ ID NO:54), MmcG (SEQ ID NO:122 encoded by SEQ ID NO:55), MmcH (SEQ ID NO:123 encoded by SEQ ID NO:56), MmcI (SEQ ID NO:124 encoded by SEQ ID NO:57), MmcJ (SEQ ID NO:125 encoded by SEQ ID NO:58), MmcK (SEQ ID NO:126 encoded by SEQ ID NO:59), MmcL (SEQ ID NO:127 encoded by SEQ ID NO:60), MmcM (SEQ ID NO:128 encoded by SEQ ID NO:61), MmcN (SEQ ID NO:129 encoded by SEQ ID NO:62), MmcO (SEQ ID NO:130 encoded by SEQ ID NO:63), MmcP (SEQ ID NO:131 encoded by SEQ ID NO:64), MmcQ (SEQ ID NO:132 encoded by SEQ ID NO:65), MmcR (SEQ ID NO:133 encoded by SEQ ID NO:66), MmcS (SEQ ID NO:134 encoded by SEQ ID NO:67), MmcT (SEQ ID NO:135 encoded by SEQ ID NO:68), MmcU (SEQ ID NO:136 encoded by SEQ ID NO:69), MmcV (SEQ ID NO:137 encoded by SEQ ID NO:70), MmcW (SEQ ID NO:138 encoded by SEQ ID NO:71), MmcX (SEQ ID NO:139 encoded by SEQ ID NO:72), MmcY (SEQ ID NO:141 encoded by SEQ ID NO:73), Mct (SEQ ID NO:117 encoded by SEQ ID NO:16), a variant or a fragment thereof, e.g., a nucleic acid molecule that hybridizes under moderate, or more preferably stringent, hybridization conditions to at least one of the nucleic acid sequences identified hereinabove or the complement thereof.

The invention further provides an isolated and purified nucleic acid molecule which is linked to a mitomycin biosynthetic gene cluster and which encodes polyketide biosynthetic enzymes, a variant or a fragment thereof. Preferably, the nucleic acid molecule of this embodiment of the invention comprises at least one, preferably at least five, and more preferably at least nine, open reading frames. More preferably, the nucleic acid molecule hybridizes under moderate, or more preferably stringent, hybridization conditions to SEQ ID NO:74, or a portion thereof.

The invention also provides an isolated and purified nucleic acid molecule which is linked to a mitomycin biosynthetic gene cluster and which encodes sugar biosynthetic enzymes, a variant or a fragment thereof. Preferably, the nucleic acid molecule of this embodiment of the invention comprises at least one, preferably at least five, more preferably at least nine, and even more preferably at least twelve, open reading frames. Preferably, the nucleic acid molecule of this embodiment of the invention hybridizes under moderate, or more preferably stringent, hybridization conditions to SEQ ID NO:75, or a portion thereof.

The invention also provides a variant polypeptide having at least about 80%, more preferably at least about 90%, and even more preferably at least about 95%, but less than 100%, contiguous amino acid sequence identity to a polypeptide having an amino acid sequence encoded by SEQ ID NO:76, or a fragment thereof. A preferred variant polypeptide includes a variant polypeptide or fragment thereof having at least about 1%, more preferably at least about 10%, and even more preferably at least about 50%, the activity of the polypeptide having the amino acid sequence comprising SEQ ID NO:10-12, 17 or 100-141. Thus, for example, the activity of a polypeptide having SEQ ID NO:98 can be compared to a variant of SEQ ID NO:98 having at least one amino acid substitution, insertion, or deletion relative to SEQ ID NO:98.

A variant nucleic acid sequence of the invention has at least about 80%, more preferably at least about 90%, and even more preferably at least about 95%, but less than 100%, contiguous nucleic acid sequence identity to a nucleic acid sequence comprising SEQ ID NO:76, the complement thereof, or a fragment thereof The amino acid and/or nucleic acid similarity (or homology) of two sequences may be determined manually or using algorithms well known to the art.

The invention also provides probes and primers comprising at least a portion of the nucleic acid molecules of the invention. The probes or primers of the invention are preferably detectably labeled or have a binding site for a detectable label. Preferably, the probes or primers of the invention are at least about 7, more preferably at least about 15, contiguous nucleotides bases having at least about 80% identity, more preferably at least about 90% identity, to the isolated nucleic acid molecules of the invention. Such probes or primers are useful to detect, quantify, isolate and/or amplify DNA strands with complementary to sequences related to the mitomycin biosynthetic gene cluster, sequences related to those encoding the polyketide biosynthetic enzymes linked to the mitomycin biosynthetic gene cluster, sequences related to those encoding sugar biosynthetic enzymes linked to the mitomycin biosynthetic gene cluster, a variant or a fragment thereof.

Also provided is an expression cassette comprising a nucleic acid molecule comprising at least a portion of a mitomycin biosynthetic gene cluster, a nucleic acid molecule which is linked to a mitomycin biosynthetic gene cluster and which encodes polyketide biosynthetic enzymes, a nucleic acid molecule which is linked to a mitomycin biosynthetic gene cluster and which encodes sugar biosynthetic enzymes, a variant or fragment thereof; operably linked to a promoter functional in a host cell. Host cells that have been modified genetically, i.e., recombinant host cells, include host cells comprising an expression cassette, e.g., an expression cassette of the invention, or host cells in which the genome has been genetically manipulated, e.g., by deletion of a portion of, replacement of a portion of, or by disruption of, the host chromosome, so as to reduce or eliminate the expression of a particular mitomycin biosynthetic gene, polyketide biosynthetic gene or a sugar biosynthetic gene of the invention.

One embodiment of the invention is a recombinant host cell, e.g., a bacterial cell, in which a portion of a nucleic acid sequence comprising the mitomycin gene cluster, i.e., the endogenous or native genomic sequence, is disrupted or replaced, for example, by an insertion with heterologous sequences or substituted with a variant nucleic acid sequence of the invention, preferably so as to result in altered mitomycin synthesis, such as an increase in mitomycin synthesis, and/or production of a novel compound. For example, the invention includes a recombinant host cell in which the mmcW gene is disrupted, for example, by replacement with a selectable marker gene, so as to yield a recombinant host cell having an increase in mitomycin production.

Another embodiment of the invention is a recombinant host cell, the genome of which is augmented by an expression cassette, e.g., via an extrachromosomal element such as a plasmid or by stable integration of the cassette into the host chromosome. Thus, the genome of the recombinant host cell is augmented with at least one mitomycin biosynthetic gene, polyketide biosynthetic gene or a sugar biosynthetic gene of the invention so as to yield an altered level of mitomycin and/or a novel compound(s) relative to the corresponding non-recombinant host cell.

Alternatively, the genome of a recombinant host cell is augmented with a non-mitomycin biosynthetic gene and, optionally, at least one mitomycin biosynthetic gene, polyketide biosynthetic gene or a sugar biosynthetic gene of the invention so as to yield an altered level of mitomycin and/or a novel compound(s) relative to the corresponding non-recombinant host cell. For example, the recombinant host cell may be augmented with pik4 (see U.S. application Ser. No. 09/105,537, filed Jun. 26, 1998, the disclosure of which is incorporated by reference herein) and piki expressed in an amount effective to yield a novel compound(s).

Host cells usefull to prepare the recombinant host cells of the invention include cells which do not express or do not comprise nucleic acid corresponding to the nucleic acid molecules of the invention, e.g., mitomycin biosynthetic genes, as well as cells which naturally produce mitomycin.

Thus, the invention also provides isolated and purified polypeptides encoded by a nucleic acid molecule of the invention. Preferably, the polypeptide of the invention is obtained from recombinant host cells, e.g., the genome of which is augmented by a nucleic acid molecule of the invention. In addition, expression cassettes and host cells comprising antisense sequences of at least a portion of the mitomycin biosynthetic gene cluster of the invention are envisioned.

In another embodiment of the invention, the isolated and purified nucleic acid molecule which is linked to a mitomycin biosynthetic gene cluster and which encodes polyketide biosynthetic enzymes, e.g., a polyketide synthase, is useful in methods to prepare recombinant polyhydroxyalkanoate monomer synthases and polymers.

Thus, the present invention provides a method of preparing a polyhydroxyalkanoate synthase. The method comprises introducing expression cassette into a host cell. The expression cassette comprises a DNA molecule encoding a polyketide synthase, operably linked to a promoter functional in the host cell. The DNA molecule is preferably obtained from a mitomycin-producing organism, e.g., a Streptomyces spp. such as S. lavendulae. The DNA molecule encoding the polyketide synthase is then expressed in the cell. Thus, another embodiment of the invention provides a purified recombinant polyketide isolated from a host cell which expresses the synthase.

Another embodiment of the invention is a method of preparing a polyhydroxyalkanoate polymer. The method comprises introducing a first expression cassette and a second expression cassette into a host cell. The first expression cassette comprises a DNA segment encoding a fatty acid synthase in which the dehydrase activity has been inactivated that is operably linked to a promoter functional in the host cell, e.g., an insect cell. The inactivation preferably is via a mutation in the catalytic site of the dehydrase. The second expression cassette comprises a DNA segment encoding a polyketide synthase that is preferably obtained from a mitomycin-producing organism operably linked to a promoter functional in the host cell. The expression cassettes may be on the same or separate molecules. The DNA segments in the expression cassettes are expressed in the cell so as to yield a polyhydroxyalkanoate polymer.

The present invention also provides an expression cassette comprising a nucleic acid molecule encoding a polyhydroxyalkanoate monomer synthase operably linked to a promoter functional in a host cell. The nucleic acid molecule comprises a plurality of DNA segments. Thus, the nucleic acid molecule comprises at least a first and a second DNA segment. The first DNA segment encodes a first module and the second DNA segment encodes a second module, wherein the DNA segments together encode a polyhydroxyalkanoate monomer synthase. No more than one DNA segment is derived from the eryA gene cluster of Saccharopolyspora erythraea. It is also preferred that the first DNA segment comprises a module from a mitomycin-producing organism, e.g., Streptomyces spp. The nucleic acid molecule may optionally firther comprise a third DNA segment encoding a polyhydroxyalkanoate synthase. Alternatively, a second nucleic acid molecule encoding a polyhydroxyalkanoate synthase may be introduced into the host cell.

Also provided is an isolated and purified DNA molecule. The DNA molecule comprises a plurality of DNA segments. Thus, the DNA molecule comprises at least a first and a second DNA segment. The first DNA segment encodes a first module and the second DNA segment encodes a second module. Together the DNA segments encode a recombinant polyhydroxyalkanoate monomer synthase. It is preferred that no more than one DNA segment is derived from the eryA gene cluster of Saccharopolyspora erythraea. Also, it is preferred that no more than one module is derived from the gene cluster from Streptomyces hygroscopicus that encodes rapamycin or the gene cluster that encodes spiramycin. A preferred embodiment of the invention employs a first DNA segment comprising a module from a mitomycin-producing organism. A further preferred embodiment of the isolated DNA molecule of the invention includes a DNA segment encoding a polyhydroxyalkanoate synthase.

Further provided is a method of preparing a polyhydroxyalkanoate polymer. The method comprises introducing a first DNA molecule and a second DNA molecule into a host cell. The first DNA molecule comprises a DNA segment encoding a recombinant polyhydroxyalkanoate monomer synthase. The recombinant polyhydroxyalkanoate monomer synthase comprises a plurality of modules. Thus, the monomer synthase comprises at least a first module and a second module. The first DNA molecule is operably linked to a promoter functional in a host cell. The second DNA molecule comprises a DNA segment encoding a polyhydroxyalkanoate synthase operably linked to a promoter functional in the host cell. It is preferred that at least one module is from a mitomycin-producing organism. The DNAs encoding the recombinant polyhydroxyalkanoate monomer synthase and polyhydroxyalkanoate synthase are expressed in the host cell so as to generate a polyhydroxyalkanoate polymer.

Yet another embodiment of the invention is an isolated and purified DNA molecule. The DNA molecule comprises a plurality of DNA segments. That is, the DNA molecule comprises at least a first and a second DNA segment. The first DNA segment encodes a fatty acid synthase and the second DNA segment encodes a module of a polyketide synthase. A preferred embodiment of the invention employs a second DNA segment comprising a module of a polyketide synthase from a mitomycin-producing organism such as Streptomyces.

Also provided is a method of providing a polyhydroxyalkanoate monomer synthase. The method comprises introducing an expression cassette into a host cell. The expression cassette comprises a DNA molecule encoding a polyhydroxyalkanoate monomer synthase operably linked to a promoter functional in the host cell. The monomer synthase comprises a plurality of modules. Thus, the monomer synthase comprises at least a first and second module which together encode the monomer synthase. A preferred embodiment of the invention employs a module from a mitomycin-producing organism. Optionally, the expression cassette further comprises a second DNA molecule encoding a polyhydroxyalkanoate synthase.

The invention also provides an isolated and purified DNA molecule comprising a first DNA segment encoding a first module and a second DNA segment encoding a second module, wherein the DNA segments together encode a recombinant polyhydroxyalkanoate monomer synthase. Preferably, at least one DNA segment is derived from DNA which is linked to the mitomycin gene cluster of S. lavendulae. Also preferably, no more than one DNA segment is derived from the eryA gene cluster of Saccharopolyspora erythraea. In one embodiment of the invention, the 3′ most DNA segment of the isolated DNA molecule of the invention encodes a thioesterase II. Also provided is an expression cassette comprising a nucleic acid molecule encoding the polyhydroxyalkanoate monomer synthase operably linked to a promoter functional in a host cell.

Yet another embodiment of the invention is a method of providing a polyhydroxyalkanoate monomer. The method comprises introducing into a host cell a DNA molecule comprising a DNA segment encoding a recombinant polyhydroxyalkanoate monomer synthase operably linked to a promoter functional in the host cell. Preferably, the second DNA molecule is derived from DNA which is linked to the mitomycin gene cluster. The recombinant polyhydroxyalkanoate monomer synthase comprises a first module and a second module, wherein at least one DNA segment is derived from DNA which is linked to a mitomycin gene cluster, e.g., the mitomycin gene cluster of S. lavendulae. The DNA encoding the recombinant polyhydroxyalkanoate monomer synthase is then expressed in the host cell so as to generate a polyhydroxyalkanoate monomer. Optionally, a second DNA molecule may be introduced into the host cell. The second DNA molecule comprises a DNA segment encoding a polyhydroxyalkanoate synthase operably linked to a promoter functional in the host cell. The two DNA molecules are expressed in the host cell so as to generate a polyhydroxyalkanoate polymer.

Another embodiment of the invention is an isolated and purified DNA molecule comprising a first DNA segment encoding a fatty acid synthase and a second DNA segment encoding a module from the DNA which is linked to the mitomycin gene cluster of S. lavendulae. Such a DNA molecule can be employed in a method of providing a polyhydroxyalkanoate monomer. Thus, a DNA molecule comprising a first DNA segment encoding a fatty acid synthase and a second DNA segment encoding a polyketide synthase is introduced into a host cell. The first DNA segment is 5′ to the second DNA segment and the first DNA segment is operably linked to a promoter functional in the host cell. The first DNA segment is linked to the second DNA segment so that the linked DNA segments express a fusion protein. The DNA molecule is expressed in the host cell so as to generate a polyhydroxyalkanoate monomer.

Further provided is a method of providing a polyhydroxyalkanoate monomer synthase. The method comprises introducing an expression cassette comprising a DNA molecule encoding a polyhydroxyalkanoate synthase operably linked to a promoter functional in a host cell. The DNA molecule comprises a first DNA segment encoding a first module and a second DNA segment encoding a second module wherein the DNA segments together encode a polyhydroxyalkanoate monomer synthase. At least one DNA segment is derived from DNA which is linked to the mitomycin gene cluster of S. lavendulae. The DNA molecule is expressed in the host cell. Optionally, the DNA molecule further comprises a DNA segment encoding a polyhydroxyalkanoate synthase. Alternatively, a second, separate DNA molecule encoding a polyhydroxyalkanoate synthase is introduced into the host cell.

Thus, the invention provides an isolated and purified DNA molecule comprising a first DNA segment encoding a first module and a second DNA segment encoding a second module, wherein the DNA segments together encode a recombinant polyhydroxyalkanoate monomer synthase, and wherein at least one DNA segment is derived from the mit/mmc gene cluster of S. lavendulae. Preferably, no more than one DNA segment is derived from the eryA gene cluster of Saccharopolyspora erythraea. In one embodiment of the invention, the 3′ most DNA segment of the isolated DNA molecule of the invention encodes a thioesterase II. Also provided is an expression cassette comprising a nucleic acid molecule encoding the polyhydroxyalkanoate monomer synthase operably linked to a promoter functional in a host cell.

Yet another embodiment of the invention is a method of providing a polyhydroxyalkanoate monomer. The method comprises introducing into a host cell a DNA molecule comprising a DNA segment encoding a recombinant polyhydroxyalkanoate monomer synthase operably linked to a promoter functional in the host cell. The recombinant polyhydroxyalkanoate monomer synthase comprises a first module and a second module, wherein at least one DNA segment is derived from the mit/mmc gene cluster of S. lavendulae. The DNA encoding the recombinant polyhydroxyalkanoate monomer synthase is then expressed in the host cell so as to generate a polyhydroxyalkanoate monomer. Optionally, a second DNA molecule may be introduced into the host cell. The second DNA molecule comprises a DNA segment encoding a polyhydroxyalkanoate synthase operably linked to a promoter functional in the host cell. The two DNA molecules are expressed in the host cell so as to generate a polyhydroxyalkanoate polymer.

Another embodiment of the invention is an isolated and purified DNA molecule comprising a first DNA segment encoding a fatty acid synthase and a second DNA segment encoding a module from the mit/mmc gene cluster of S. lavendulae. Such a DNA molecule can be employed in a method of providing a polyhydroxyalkanoate monomer. Thus, a DNA molecule comprising a first DNA segment encoding a fatty acid synthase and a second DNA segment encoding a polyketide synthase is introduced into a host cell. The first DNA segment is 5′ to the second DNA segment and the first DNA segment is operably linked to a promoter functional in the host cell. The first DNA segment is linked to the second DNA segment so that the linked DNA segments express a fusion protein. The DNA molecule is expressed in the host cell so as to generate a polyhydroxyalkanoate monomer.

Further provided is a method of providing a polyhydroxyalkanoate monomer synthase. The method comprises introducing an expression cassette comprising a DNA molecule encoding a polyhydroxyalkanoate synthase operably linked to a promoter functional in a host cell. The DNA molecule comprises a first DNA segment encoding a first module and a second DNA segment encoding a second module wherein the DNA segments together encode a polyhydroxyalkanoate monomer synthase. At least one DNA segment is derived from the mit/mmc gene cluster of S. lavendulae. The DNA molecule is expressed in the host cell. Optionally, the DNA molecule further comprises a DNA segment encoding a polyhydroxyalkanoate synthase. Alternatively, a second, separate DNA molecule encoding a polyhydroxyalkanoate synthase is introduced into the host cell.

Also provided is a method for directing the biosynthesis of specific sugar-modified polyketides by genetic manipulation of a polyketide-producing microorganism. The method comprises introducing into a polyketide-producing microorganism a DNA sequence encoding enzymes in sugar biosynthesis, e.g., a DNA sequence comprising SEQ ID NO:75, a variant or fragment thereof, so as to yield a microorganism that produces specific sugar-modified polyketides. Alternatively, an anti-sense DNA sequence of the invention may be employed. Then the sugar-modified polyketides are isolated from the microorganism. It is preferred that the DNA sequence is modified so as to result in the inactivation of at least one enzymatic activity in sugar biosynthesis or in the attachment of the sugar to a polyketide

Thus, the modules encoded by the nucleic acid segments of the invention may be employed in the methods described hereinabove to prepare polyhydroxyalkanoates of varied chain length or having various side chain substitutions.

The compounds produced by the recombinant host cells of the invention are preferably biologically active agents such as antibiotics, anti-inflammatory agents, anti-cancer agents, antibiotics, immune-enhancers, immunosuppressants, agents to treat asthma, chronic obstructive pulmonary disease as well as other diseases involving respiratory inflammation, or cholesterol-lowering agents; or as crop protection agents (e.g., fungicides or insecticides), as well as biopolymers, e.g., in packaging or biomedical applications, or to engineer PHA monomer synthases. Methods employing these compounds, e.g., to treat a mammal, e.g., a human, bird or fish in need of such therapy, are also envisioned.

### BRIEF DESCRIPTION OF THE FIGURES

FIG. 1. The biosynthetic pathway for mitomycin antibiotics.

FIG. 2. Organization of the mitomycin gene cluster. The deduced ORFs are drawn to scale, and their corresponding genes are marked in italics. The filled bars indicate the location of the mitomycin cluster. Abbreviations of the restriction enzymes: B: BamHI, S: SphI, P: PstI, E: EcoRI, X: XhoI, K: KpnI.

FIG. 3. The three SAM dependent methyltransferase conserved motifs can be found in MitM (SEQ ID NO:1), MitN (SEQ ID NO:2), and MmcR (SEQ ID NO:3). DmpM (SEQ ID NO:4; Kim et al., 1998), TcmN (SEQ ID NO:5; Shikano et al., 1998), ORF14 (SEQ ID NO:6; August et al., 1998), EryG (SEQ ID NO:7; Hardwick and Pelham, 1994) are O-methyltransferases for puromycin, tetracenomycin C, rifamycin, and erytliromycin biosynthesis, respectively. Consen=consensus sequence (SEQ ID NO:8).

FIG. 4. Sequence similarity of MitM, MitN, and MmcR with other O-methyltransferases: DmpM (Kim et al., 1998), TcmN (Shikano et al., 1998), ORF14 (August et al., 1998), EryG (Hardwick and Pelham, 1994), RdmB (Mazodier et al., 1989), DnrK (Lee and Stock, 1996), and DauK (Devereux et al. 1984)); and C-methyltransferases: SMT (Schafeijohann et al., 1993), ESMTI (Floss, 1997), SMT1 (Blattner et al., 1997), and SED6 (Guilfoile and Hutchinson, 1992)). The dendrogram was constructed with the program PILEUP (Denis and Brzezinld, 1992).

FIG. 5. MC genes and deduced enzme functions.

FIG. 6. Bacterial strains and plasmids. Strains DH5α and DH5αF′ are available from Gibco BRL (Gaithersburg, MD), ATCC 27643 and NRRL 2564 are available from the American Type Culture Collection, and strain S17-1 is described in Hidaka et al. (1995). Plasmids pNJ1, pUC119, pKC 1139, pDHS3001, pKN108, and pFD666 are described in Kuzuyama et al. (1995), Madduri et al. (1993), Boxer (1997), Kagan and Clarke (1994), Kim et al. (1998), and Coque et al. (1995), respectively.

FIG. 7. Biosynthetic pathway leading to mitomycin C.

FIG. 8. Southern hybridization and restriction-enzyme map of the mrd and rifK hybridizing regions from S. lavendulae. A) Southern hybridization with the rifK gene probe (Kim et al., 1998). Lane 1, A. mediteranei ATCC 27643 genomic DNA digested with BamHI; Lane 2, S. lavendulae NRRL 2564 genomic DNA digested with BamHI; B) Physical map showing the mitABC genes. The location of mrd and rifK hybridizing genes in cosmid pDHS7529 are indicated by solid bars. Enzymes: E, EcoRI; B, BamHI. The sequenced 3.8 kb BamHI fragment containing mitA, mitB, mitC is enlarged (wide arrows). Thin arrows below show sites of resistance gene integration for disruption experiments.

FIG. 9. Nucleotide sequence of the 3.8 kb DNA fragment containing mitABC (SEQ ID NO:9). The deduced gene products are indicated in the one-letter code under the DNA sequence (SEQ ID NO:10, MitA; SEQ ID NO: 11, MitB; SEQ ID NO:12, MitC). Possible ribosome binding sites are marked in the boxed regions. The presumed translational start site and direction of transcription for each ORF is indicated by an arrow and marked accordingly.

FIG. 10. Alignment of MitA with three other AHBA synthases. The deduced amino acid sequence comparison from AHBAS genes derived from Streptomyces lavendulae (SEQ ID NO:10). Streptomyces collinus (Z54208; SEQ ID NO:13), Actinosynnema pretiosum (I39657; SEQ ID NO:14), and Amycolatopsis mediteranei (I39657; SEQ ID NO:15) is shown with the conserved lysine in the PLP-binding motif underlined.

FIG. 11. Southern blot analysis of the mitA mutant strain. A) Construction of mitA disruption mutant and restriction map of the wild-type and mitA disruption mutant showing expected band sizes in the Southern blot. Maps are not drawn to scale. B) S. lavendulae genomic DNA from wild-type (lanes 1 and 2) and double crossover mutant (lanes 3 and 4) were digested with BamHI (lane 1 and 3) and SphI (lane 2 and lane 4), respectively. The 4.9 kb EcoRI-HindIII fragment from pDHS2001 containing tsr-disrupted mitA was used as the probe.

FIG. 12. Southern blot analysis of mitB mutant MM101. A) Construction of mitB disruption mutant and restriction map of the wild-type and mitB disruption mutant showing the expected sites in the Southern blot. B) S. lavendulae genomic DNA from wild-type (lane 1 and 3) and mitB mutant (lane 2 and 4) were digested with BamHI (lane 1 and 2) and Sacl (lane 3 and 4). DNA probe: 3.8 kb BamHI fragment insert from pDHS7601.

FIG. 13. Chemical analysis and biological activity of extracts from S. lavendulae wild-type and mutant strains. A) HPLC analysis of authentic mitomycin C standard, mitomycin C production in the wild-type S. lavendulae, mitA (AHBAS) and mitB (gtf) disruption mutants of S. lavendulae. One mg of crude extract injected, 1 μg of MC injected as standard. B) Bacillus subtilis bioassay of mitomycin C production in mitA disruption mutant strain of S. lavendulae. Filter discs: 1) 100 μg injection of wild-type—collected 12.5-13.5 minutes; 2) 100 μg injection of mitA (ahbas) disruption mutant—collected 12.5-13.5 minutes; 3) 100 μg injection of W.T. containing vector—collected 12.5-13.5 minutes; 4) One μg of mitomycin C collected from HPLC from 12.5-13.5 minutes; 5) Tris buffer negative control; 6) methanol solvent negative control.

FIG. 14. Strains and plasmids employed in Example 3. BL21 (DE3) and pET17b are available from Novagen (Madison, Wis.). pDH57006 is described in Sheldon et al. (1997).

FIG. 15. Genetic map showing the physical linkage of the mct and mrd genes within the MC biosynthetic gene cluster. The expanded box shows the line plot of the mct ORF.

FIG. 16. The nucleotide sequence of mot (SEQ ID NO:16). The deduced amino acid sequence of mct is indicated under the nucleotide sequence with the one letter designation (SEQ ID NO:17). A conserved motif characteristic of 14 TMS proteins is boxed while the invariant beta-turn motif is denoted with a dashed underline. The putative ribosome binding site is marked with a solid underline.

FIG. 17. Dot matrix alignment of the deduced amino acid sequence of mct with other actinomycete antibiotic efflux proteins. Comparable parameters were utilized in generating the alignments.

FIG. 18. Hydropathy analysis of the deduced amino acid sequence of MC-translocase. A) Hydropathy plot obtained from prediction of Kyte and Doolittle (1982). B) Schematic representation of MC-translocase protein topology. The trnnsmembrane spanning regions are marked (1-14). The initial and final amino acid positions of each transmembrane domain are indicated by small numbers. The relative position of positively (H, R, K) and negatively (D, Q) charged amino acids are indicated by a plus and minus, respectively.

FIG. 19. Creation of the mct disruption mutant. A) The chromosomal mct gene (black bar) was disrupted by inserting a neomycin resistance marker (shaded) within the gene. Following double crossover recombination, specific restriction bands are predicted to be shifted in the mct mutant genome compared to the wild-type strain. B) Southem blot analysis of the mct mutant. As expected, when probed with the 4.0 kb Bamli insert from pDHS7661, the 4.0 kb BamHI hybridization band in wild-type S. lavendulae was shifted to 5.4 kb in mct knockouts, while a 1.65 kb SacI hybridization band was shifted to 3.0 kb in size. Lane 1 and 5: wild-type genomic DNA digested with BamHI. Lane 2, 3, 4, and 6: Four double crossover colonies genomic DNA digested with BamHI. Lane 7: wild-type genomic DNA digested with SstI. Lane 8: double crossover clone 6 genomic DNA digested with SstI.

FIG. 20. MC uptake analysis of strains PJS100, PJS102, and PJS103. BL21(DE3)::pET17b vector control strain, (●); strain PJS100, (▪); strain PJS102, (♦); strain PJS103, (x).

FIG. 21. Complete nucleotide sequence of the mitomycin gene cluster (SEQ ID NO:96).

FIG. 22. Complete nucleotide sequence of ORFs 1-9 (SEQ ID NO:74).

FIG. 23. Complete nucleotide sequence of ORFs 11-22 (SEQ ID NO:75).

FIG. 24. Codons for various amino acids.

FIG. 25. Exemplary amino acid substitutions.

FIG. 26. Complete nucleotide sequence of the mitomycin biosynthetic genes (SEQ ID NO:76).

FIG. 27A. Structure of selected mitomycins.

FIG. 27B. Structure of selected naturally occurring mitomycins.

FIG. 28. Synthetic scheme.

FIG. 29. ¹H spectrum of 9a-demethoxy mitomycin A (CD₃CN, 800 MHZ).

FIG. 30. ¹H-¹H COSY spectrm of 9a-demethoxy mitomycin A (CD₃CN, 800 MHZ).

FIG. 31. HMQC spectrum of 9a-demethoxy mitomycin A (CD₃CN, 800 MHZ).

FIG. 32. HMBC spectrum (2.4 ppm to 4.9 ppm) of 9a-demethoxy mitomycin A (CD₃CN, 800 MHZ).

FIG. 33. HMBC spectrum (1.6 ppm to 2.35 ppm) of 9a-demethoxy mitomycin A (CD₃CN, 800 MHZ).

FIG. 34. Low and high resolution electrospray ionization mass spectrum of 9a-demethoxy mitomycin A.

FIG. 35, UV spectrum of 9ademethoxy mitomycin A.

FIG. 36. ¹H spectrum of 9-epi-mitomycin B in CD₃CN and CD₃0D, 800 MHZ.

FIG. 37. Low and high resolution electrospray ionization mass spectrum of 9-epi-mitomycin B.

FIG. 38. Schematic of the construction of a mitM deletional mutant.

FIG. 39. MC production in wild type and various mitM and mitN mutants.

FIG. 40. Purification of MitM and MitN.

FIG. 41. Conversion of MA to MF by MitM and MitN.

FIG. 42. Late stages in mitomycin biosynthesis.

FIG. 43. Thin layer chromatogram of MM107 and MM108.

### DETAILED DESCRIPTION OF THE INVENTION

Definitions

As used herein, a “Type I polyketide synthase” is a single polypeptide with a single set of iteratively used active sites. This is in contrast to a Type II polyketide synthase which employs active sites on a series of polypeptides.

As used herein, a “linker region” is an amino acid sequence present in a multifunctional protein which is less well conserved in an amino acid sequence than an amino acid sequence with catalytic activity.

As used herein, an “extender unit” catalytic or enzymatic domain is an acyl transferase in a module that catalyzes chain elongation by adding 2-4 carbon units to an acyl chain and is located carboxy-terminal to another acyl transferase. For example, an extender unit with methylmalonylCoA specificity adds acyl groups to a methylmalonylCoA molecule.

As used herein, a “polyhydroxyalkanoate” or “PHA” polymer includes, but is not limited to, linked units of related, preferably heterologous, hydroxyalkanoates such as 3-hydroxybutyrate, 3-hydroxyvalerate, 3-hydroxycaproate, 3-hydroxyheptanoate, 3-hydroxyhexanoate, 3-hydroxyoctanoate, 3-hydroxyundecanoate, and 3-hydroxydodecanoate, and their 4-hydroxy and 5-hydroxy counterparts.

As used herein, a “recombinant” nucleic acid or protein molecule is a molecule where the nucleic acid molecule which encodes the protein has been modified in vitro, so that its sequence is not naturally occurring, or corresponds to naturally occurring sequences that are not positioned as they would be positioned in a genome which has not been modified.

As used herein, a “multifunctional protein” is one where two or more enzymatic activities are present on a single polypeptide.

As used herein, a “module” is one of a series of repeated units in a multifunctional protein, such as a Type I polyketide synthase or a fatty acid synthase.

As used herein, a “premature termination product” is a product which is produced by a recombinant multifunctional protein which is different than the product produced by the non-recombinant multifunctional protein. In general, the product produced by the recombinant multifunctional protein has fewer acyl groups.

As used herein, a DNA that is “derived from” a gene cluster is a DNA that has been isolated and purified in vitro from genomic DNA, or synthetically prepared on the basis of the sequence of genomic DNA.

An “antibiotic” as used herein is a substance produced by a microorganism which, either naturally or with limited chemical modification, will inhibit the growth of or kill another microorganism or eukaryotic cell.

An “antibiotic biosynthetic gene” is a nucleic acid, e.g., DNA, segment or sequence that encodes an enzymatic activity which is necessary for an enzymatic reaction in the process of converting primary metabolites into antibiotics.

An “antibiotic biosynthetic pathway” includes the entire set of antibiotic biosynthetic genes necessary for the process of converting primary metabolites into antibiotics. These genes can be isolated by methods well known to the art, e.g., see U.S. Pat. No. 4,935,340.

Antibiotic-producing organisms include any organism, including, but not limited to, Actinoplanes, Actinomadura, Bacillus, Cephalosporium, Micromonospora, Penicillium, Nocardia, and Streptomyces, which either produces an antibiotic or contains genes which, if expressed, would produce an antibiotic.

The term “polyketide” as used herein refers to a large and diverse class of natural products, including but not limited to antibiotic, antifingal, anticancer, and anti-helminthic compounds.

The term “polyketide-producing microorganism” as used herein includes any microorganism that can produce a polyketide naturally or after being suitably engineered (i.e., genetically). Examples of actinomycetes that naturally produce polyketides include but are not limited to Micromonospora rosaria, Micromonospora megalomicea, Saccharopolyspora erythraea, Streptomyces antibioticus, Streptomyces albereticuli, Streptomyces ambofaciens, Streptomyces avermitilis, Streptomycesfradiae, Streptomyces griseus, Streptomyces hydroscopicus, Streptomyces tsukulubaensis, Streptomyces mycarofasciens, Streptomyces platenesis, Streptomyces violaceoniger, Streptomyces violaceoniger, Streptomyces thermotolerans, Streptomyces rimosus, Streptomyces peucetius, Streptomyces coelicolor, Streptomes glaucescens, Streptomyces roseofulvus, Streptomyces cinnamonensis, Streptomyces curacoi, and Amycolatopsis mediteranei (see Hopwood, D. A and Sherman, D. H., Annu. Rev. Genet., 24:37-66 (1990), incorporated herein by reference). Other examples of polyketide-producing microorganisms that produce polyketides naturally include various Actinomadura, Dactylosporangium and Nocardia strains.

The term “glycosylated polyketide” refers to any polyketide that contains one or more sugar residues.

The term “glycosylation-modified polyketide” refers to a polyketide having a changed glycosylation pattern or configuration relative to that particular polyketide's unmodified or native state.

The term “sugar biosynthesis genes” as used herein refers to nucleic acid sequences from organisms such as S. lavendulae that encode sugar biosynthesis enzymes and is intended to include sequences of DNA from other polyketide-producing microorganisms which are identical or analogous to those obtained from S. lavendulae.

The term “sugar biosynthesis enzymes” as used herein refers to polypeptides which are involved in the biosynthesis and/or attachment of polyketide-associated sugars and their derivatives and intermediates.

The term “polyketide-associated sugar” refers to a sugar that is known to attach to polyketides or that can be attached to polyketides by the processes described herein.

The term “sugar derivative” refers to a sugar which is naturally associated with a polyketide but which is altered relative to the unmodified or native.

The term “sugar intermediate” refers to an intermediate compound produced in a sugar biosynthesis pathway.

A “recombinant” host cell of the invention has a genome that has been manipulated in vitro so as to alter, e.g., decrease or disrupt, or, alternatively, increase, the function or activity of at least one gene, e.g., in the mitomycin biosynthetic gene cluster, of the invention.

As used herein, the “mit/mmc” or “mitomycin” gene cluster includes sequences encoding enzymes for mitosane precursor formation, mitosane ring assembly, regulation of mitomycin biosynthesis, functionalization, and resistance to mitomycin, as well as closely linked sequences encoding polyketide and sugar biosynthetic enzyes.

As used herein, the terms “isolated and/or purified” refer to in vitro isolation of a RNA, DNA or polypeptide molecule from its natural cellular environment, and from association with other components of the cell, such as nucleic acid or polypeptide, so that is can be sequenced, replicated and/or expressed. Moreover, the nucleic acid may encode more than one polypeptide. For example, “an isolated DNA molecule encoding an AHBA synthase” is RNA or DNA containing greater than 7, preferably 15, and more preferably 20 or more sequential nucleotide bases that preferably encode a biologically active polypeptide, or a fragment or variant thereof, that is complementary to the non-coding, or complementary to the coding strand, of an AHBA synthase RNA, or hybridizes to the RNA or DNA encoding the AHBA synthase and remains stably bound under low, moderate, or stringent conditions, as defined by methods well known to the art, e.g., in Sambrook et al., supra.

An antibiotic resistance-conferring gene is a nucleic acid segment that encodes an enzymatic or other activity which alone or in combination with other gene products, confers resistance to an antibiotic.

As used herein, “mitomycin” includes, but is not limited to, mitomycin A, mitomycin B, mitomycin C, porfiromycin, mitiromycin, mitomycin D, mitomycin E, mitomycin F, mitomycin G, mitomycin H, mitomycin I, mitomycin J, mitomycin L, mitomycin M, mitomycin K, albomitomycin A, isomitomycin A, KW2149, KW2149 metabolites such as M-16 and M-18, FR66979, FK973, FK317, and FR900482, as well as structural or functional equivalents thereof (“analogs”), or derivatives thereof.

As used herein, the term “derivative” means that a particular compound produced by a host cell of the invention or prepared in vitro using polypeptides encoded by the nucleic acid molecules of the invention, is modified so that it comprises other moieties, e.g., peptide or polypeptide molecules, such as antibodies or fragments thereof, nucleic acid molecules, sugars, lipids, fats, a detectable signal molecule such as a radioisotope, e.g., gamma emitters small chemicals, metals, salts, synthetic polymers, e.g., polylactide and polyglycolide, surfactants and glycosaminoglycans, which are covalently or non-covalently attached or linked to the compound.

It will be appreciated by those skilled in the art that each atom of the compounds of the invention having a chiral center may exist in and be isolated in optically active and racemic forms. Some compounds may exhibit polymorphism. It is to be understood that the present invention encompasses any racemic, optically active, polymorphic or stereoisomeric form, or mixtures thereof, of a compound of the invention, which possess the useful properties described herein, it being well known in the art how to prepare optically active forms (for example, by resolution of the racemic form by recrystallization techniques, by synthesis from optically active starting materials, by chiral synthesis, or by chromatographic separation using a chiral stationary phase) and how to determine activity using the standard tests described herein, or using other similar tests which are well known in the art.

The term “sequence homology” or “sequence identity” means the proportion of base matches between two nucleic acid sequences or the proportion amino acid matches between two amino acid sequences. When sequence homology is expressed as a percentage, e.g., 50%, the percentage denotes the proportion of matches over the length of sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes, the sequence homology between the target nucleic acid and the oligonucleotide sequence is generally not less than 17 target base matches out of 20 possible oligonucleotide base pair matches (85%); preferably not less than 9 matches out of 10 possible base pair matches (90%), and more preferably not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, 1972, volume 5, National Biomedical Research Foundation, pp. 101-110, and Supplement 2 to this volume, pp. 1-10. The two sequences or parts thereof are more preferably homologous if their amino acids are greater than or equal to 50% identical when optimally aligned using the ALIGN program.

The following terms are used to describe the sequence relationships between two or more polynucleotides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing, or may comprise a complete cDNA or gene sequence. Generally, a reference sequence is at least 20 nucleotides in length, frequently at least 25 nucleotides in length, and often at least 50 nucleotides in length. Since two polynucleotides mayeach (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window”, as used herein, refers to a conceptual segment of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (1981) Adv. Appi. Math. 2:482, by the homology alignment algorithm of Needleman and Wunsch (1970) J. Mol. Biol. 4& 443, by the search for similarity method of Pearson and Lipman (1988) Proc. Natl. Acad. Sci. (U.S.A.) 85: 2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

As applied to polypeptides, the term “substantial identity” means that two peptide sequences, when optimally aligned, such as by the programs GAP or BESTFIT using default gap weights, share at least about 80 percent sequence identity, preferably at least about 90 percent sequence identity, more preferably at least about 95 percent sequence identity, and most preferably at least about 99 percent sequence identity.

In accordance with the present invention, there is provided a purified and isolated nucleic acid molecule which encodes the entire pathway for the biosynthesis of mitomycin, as well as polyketide biosynthetic and sugar biosynthetic genes that are linked to the mitomycin biosynthetic genes. Desirably, the nucleic acid molecule is a DNA isolated from Streptomyces spp. The present invention fuirther includes isolated and purified DNA sequences which hybridize under standard or stringent conditions to the the nucleic acid molecules of the invention. It should be understood to those skilled in the art that the invention also encompasses the purified and isolated polypeptides which may be encoded by the sequences of the nucleic acid molecules of this invention.

The invention described herein can be used for the production of mitomycin, analogs or derivatives thereof, or novel compounds. Commercial chemical syntheses of mitomycin are not feasible. The gene cluster described herein contains all the genes required for the production of the mitosane group of antibiotics, compounds which are clinically prescribed antitumor compounds employed in the treatment of a wide variety of cancers including non-small cell lung cancer, metastatic breast cancer, esophageal, gastric, pancreatic, and anal canal carcinomas. Thus, the isolation and characterization of this gene cluster allows for the selective production of mitomycin antibiotics, the overproduction or under production of particular compounds, e.g., overproduction of certain mitomycin antibiotics, and the production of novel compounds, e.g., mitomycin-derived compounds as well as the production of novel non-mitomycin related compounds. For example, combinational biosynthetic-based modification of mitomycin antibiotics may be accomplished by selective activation or disruption of specific genes within the cluster or incorporation of the genes into biased biosynthetic libraries which are assayed for a wide range of biological activities, to derive greater chemical diversity in the mitomycins. A further example includes the introduction of a mitomycin biosynthetic gene(s) into a particular host cell so as to result in the production of a novel non-mitomycin related compound due to the activity of the mitomycin biosynthetic gene(s) on other metabolites, intermediates or components of the host cells. The in vitro expression of polypeptides from this gene cluster also provides an enzymatic route to the production of known mitomycin compounds that are produced in low quantities, or conversion of currently available mitomycins to other known or novel mitomycins, e.g., the bioconversion of mitomycin C to porfiromycin.

The mitomycin resistance genes may also be used to provide higher mitomycin resistance to cancer patients undergoing treatment and for clonal selection purposes (e.g., using mrd). For example, the resistance gene(s) may be inserted into human bone marrow cell lines to confer higher resistance to non-cancerous cells, thus allowing higher doses of mitomycins to be administered to cancer patients. Moreover, because mitomycin acts directly upon DNA itself, its toxicity is extremely broad, and therefore the resistance genes could be used for efficient selection in prokaryotes, fungi, plants, mammalian cell culture, and insect cell culture. Further, the regulatory resistance and transport genes may be used to create higher producing strains capable of synthesizing more mitomycin than can currently be obtained through traditional fermentation strategies.

In addition, the invention described herein can be used for the production of novel compounds which include a diverse range of biodegradable PHA polymers through genetic redesign of DNA such as that found in Streptomyces spp. Different PHA synthases can thenbe tested for their ability to polymerize the monomers produced by the recombinant PHA synthase into a biodegradable polymer. PHA synthases can be tested for their specificity with respect to different monomer substrates by methods well known to the art.

The potential uses and applications of PHAs produced by PHA monomer synthases and PHA synthases include both medical and industrial applications. Medical applications of PHAs include surgical pins, sutures, staples, swabs, wound dressings, blood vessel replacements, bone replacements and plates, stimulation of bone growth by piezoelectric properties, and biodegradable carrier for long-term dosage of pharmaceuticals. Industrial applications of PHAs include disposable items such as baby diapers, packaging containers, bottles, wrappings, bags, and films, and biodegradable carriers for long-teni dosage of herbicides, fungicides, insecticides, or fertilizers.

In animals, the biosynthesis of fatty acids de novo from malonyl-CoA is catalyzed by FAS. For example, the rat FAS is a homodimer with a subunit structure consisting of 2505 amino acid residues having a molecular weight of 272,340 Da. Each subunit consists of seven catalytic activities in separate physical domains (Amy et al., Proc Natl. Acad. Sci. USA, 3114 (1989)). The physical location of six of the catalytic activities, ketoacyl synthase (KS), malonyl/acetyltransferase (M/AT), enoyl reductase (ER), ketoreductase (KR), acyl carrier protein (ACP), and thioesterase (TE), has been established by (1) the identification of the various active site residues within the overall amino acid sequence by isolation of catalytically active fragments from limited proteolytic digests of the whole FAS, (2) the identification of regions within the FAS that exhibit sequence similarity with various monofunctional proteins, (3) expression of DNA encoding an amino acid sequence with catalytic activity to produce recombinant proteins, and (4) the identification of DNA that does not encode catalytic activity, i.e., DNA encoding a linker region. (Smith et al., Proc. Natl Acad Sci. USA, 73, 1184 (1976); Tsukamoto et al., J. Biol. Chem., 2M, 16225 (1988); Rangan et al., J. Biol Cem, 266, 19180 (1991)).

The seventh catalytic activity, dehydrase (DH), was identified as physically residing between AT and ER by an amino acid comparison of FAS with the amino acid sequences encoded by the three open reading frames of the eryA polyketide synthase (PKS) gene cluster of Saccharopolyspora erythraea. The three polypeptides that comprise this PKS are constructed from “modules” which resemble animal FAS, both in terms of their amino acid sequence and in the ordering of the constituent domains (Donadio et al., Gene, 111, 51 (1992); Benh et al., Fur. J. Biochem., 204, 39 (1992)).

One embodiment of the invention employs a FAS in which the DH is inactivated (FAS DH-). The FAS DH-employed in this embodiment of the invention is preferably a eukaryotic FAS DH- and, more preferably, a mammalian FAS DH-. The most preferred embodiment of the invention is a FAS where the active site in the DH has been inactivated by mutation. For example, Joshi et al. (J. Biol. Chem., 268, 22508 (1993)) changed the His⁸⁷⁸ residue in the rat FAS to an alanine residue by site-directed mutagenesis. In vitro studies showed that a FAS with this change (ratFAS206) produced 3-hydroxybutyrylCoA as a premature termination product from acetyl-CoA, malonyl-CoA and NADPH.

A FAS DH-effectively replaces the β-ketothiolase and acetoacetyl-CoA reductase activities of the natural pathway by producing D(−)-3-hydroxybutyrate as a premature termination product, rather than the usual 16-carbon product, palmitic acid. This premature termination product can then be incorporated into PHB by a PHB synthase.

Another embodiment of the invention employs a recombinant Streptomyces spp. PKS to produce a variety of β-hydroxyCoA esters that can serve as monomers for a PHA synthase. One example of a DNA encoding a Type I PKS is the eryA gene cluster, which governs the synthesis of erytliromycin aglycone deoxyeiythronolide B (DEB). The gene cluster encodes six repeated units, termed modules or synthase units (SUs). Each module or SU, which comprises a series of putative FAS-like activities, is responsible for one of the six elongation cycles required for DEB formation. Thus, the processive synthesis of asymmetric acyl chains found in complex polyketides is accomplished through the use of a programmed protein template, where the nature of the chemical reactions occurring at each point is determined by the specificities in each SU.

Two other Type I PKS are encoded by the tyl (tylosin) and met (methymycin) gene clusters (see U.S. application Ser. No. 09/108,537, the disclosure of which is incorporated by reference herein). The macrolide multifunctional synthases encoded by tyl and met provide a greater degree of metabolic diversity than that found in the eryA gene cluster. The PKSs encoded by the eryA gene cluster only catalyze chain elongation with methylmalonylCoA, as opposed to tyl and met PKSs, which catalyze chain elongation with malonylCoA, methylmalonylCoA and ethylmalonylCoA. Specifically, the tyl PKS includes two malonylCoA extender units and one ethylmalonylCoA extender unit, and the met PKS includes one malonylCoA extender unit.

In order to manipulate the catalytic specificities within each module, DNA encoding a catalytic activity must remain undisturbed. To identify the amino acid sequences between the amino acid sequences with catalytic activity, the “linker regions,” amino acid sequences of related modules, preferably those encoded by more than one gene cluster, are compared. Linker regions are amino acid sequences which are less well conserved than amino acid sequences with catalytic activity. Witkowski et al., Eur. J. Biochem., 198, 571 (1991).

In an alternative embodiment of the invention, to provide a DNA encoding a Type I PKS module with a TE and lacking a functional DH, a DNA encoding a module F, containing KS, MT, KR, ACP, and TE catalytic activities, is introduced at the 3′ end of a DNA encoding a first module. Module F introduces the final (R)-3-hydroxyl acyl group at the final step of PHA monomer synthesis, as a result of the presence of a TE domain. DNA encoding a module F is not present in the eryA PKS gene cluster (Donadio et al., supra, 1991).

A DNA encoding a recombinant monomer synthase is inserted into an expression vector. The expression vector employed varies depending on the host cell to be transformed with the expression vector. That is, vectors are employed with transcription, translation and/or post-translational signals, such as targeting signals, necessary for efficient expression of the genes in various host cells into which the vectors are introduced. Such vectors are constructed and transformed into host cells by methods well known in the art. See Sambrook et al., Molecular Cloning: A Laboratory Manual, Cold Spring Harbor (1989). Preferred host cells for the vectors of the invention include insect, bacterial, and plant cells. Preferred insect cells include Spodoptera frugiperda cells such as Sf21, and Trichoplusia ni cells. Preferred bacterial cells include Escherichia coli, Streptomyces and Pseudomonas. Preferred plant cells include monocot and dicot cells, such as maize, rice, wheat, tobacco, legumes, carrot, squash, canola, soybean, potato, and the like.

Moreover, the appropriate subcellular compartment in which to locate the enzyme in eukaryotic cells must be considered when constructing eukaryotic expression vectors. Two factors are important: the site of production of the acetyl-CoA substrate, and the available space for storage of the PHA polymer. To direct the enzyme to a particular subcellular location, targeting sequences may be added to the sequences encoding the recombinant molecules.

The baculovirus system is particularly amenable to the introduction of DNA encoding a recombinant FAS or a PKS monomer synthase because an increasing variety of transfer plasmids are becoming available which can accommodate a large insert, and the virus can be propagated to high titers. Moreover, insect cells are adapted readily to suspension culture, facilitating relatively large-scale recombinant protein production. Further, recombinant proteins tend to be produced exclusively as soluble proteins in insect cells, thus, obviating the need for refolding, a task that might be particularly daunting in the case of a large multifunctional protein. The Sf21/baculovirus system has routinely expressed milligram quantities of catalytically active recombinant fatty acid synthase. Finally, the baculoviruslinsect cell system provides the ability to construct and analyze different synthase proteins for the ability to polymerize monomers into unique biodegradable polymers.

A further embodiment of the invention is the introduction of at least one DNA encoding a PHA synthase and a DNA encoding a PHA monomer synthase into a host cell. Such synthases include, but are not limited to, A. eutrophus 3-hydroxy, 4-hydroxy, and 5-hydroxy alkanoate synthases, Rhodococcus ruber C₃-C₅ hydroxyalkanoate synthases, Pseudomonas oleororans C₆-C₁₄ hydroxyalkanoate synthases, P. putida C₆-C₁₄ hydroxyalkanoate synthases, P. aeruginosa C₅-C₁₀ hydroxyalkanoate synthases, P. resinovorans C₄-C₁₀ hydroxyalkanoate synthases, Rhodospirillum rubrum C₄-C₇ hydroxyalkanoate syntheses, R. gelatinorus C₄-C₇, Thiocapsa pfennigii C₄-C₈ hydroxyalkanoate synthases, and Bacillus megaterium C₄-C₅ hydroxyalkanoate synthases.

The introduction of DNA(s) encoding more than one PHA synthase may be necessary to produce a particular PHA polymer due to the specificities exhibited by different PHA synthases. As multifunctional proteins are altered to produce unusual monomeric structures, synthase specificity may be problematic for particular substrates. Although the A. eutrophus PHB synthase utilizes only C4 and C5 compounds as substrates, it appears to be a good prototype synthase for initial studies since it is known to be capable of producing copolymers of 3-hydroxybutyrate and 4-hydroxybutyrate (Kunioka et al., Macromolecules, 22, 694 (1989)) as well as copolymers of 3-hydroxyvalerate, 3-hydroxybutyrate, and 5-hydroxyvalerate (Doi et al., Macromolecules, 19, 2860 (1986)). Other synthases, especially those of Pseudomonas aeruginosa (Timm et al., Eur. J. Biochem., 209, 15 (1992)) and Rhodococcus ruber (Pieper et al., FEMS Microbiol. Lett., 96, 73 (1992)), can also be employed in the practice of the invention. Synthase specificity may be alterable through molecular biological methods.

In yet another embodiment of the invention, a DNA encoding a FAS and a PRA synthase can be introduced into a single expression vector, obviating the need to introduce the genes into a host cell individually.

A further embodiment of the invention is the generation of a DNA encoding a recombinant multifunctional protein, which comprises a FAS, of either eukaryotic or prokaryotic origin, and a PKS module F. Module F will carry out the final chain extension to include two additional carbons and the reduction of the β-keto group, which results in a (R)-3-hydroxy acyl CoA moiety.

To produce this recombinant protein, DNA encoding the FAS TE is replaced with a DNA encoding a linker region which is normally found in the ACP-KS interdomain region of bimodular ORFs. DNA encoding a module F is then inserted 3′ to the DNA encoding the linker region. Different linker regions, such as those described below which vary in length and amino acid composition, can be tested to determine which linker most efficiently mediates or allows the required transfer of the nascent saturated fatty acid intermediate to module F for the final chain elongation and keto reduction steps. The resulting DNA encoding the protein can then be tested for expression of long-chain β-hydroxy fatty acids in insect cells, such as Sf21 cells, or Streptomyces, or Pseudomonas. The expected 3-hydroxy C-18 fatty acid can serve as a potential substrate for PHA synthases which are able to accept long-chain alkyl groups. A preferred embodiment of the invention is a FAS that has a chain length specificity between 4-22 carbons.

Examples of linker regions that can be employed in this embodiment of the invention include, but are not limited to, the ACP-KS linker regions encoded by the tyl ORFI (ACP₁-KS₂; ACP₂-KS₃), and ORF3 (ACP₅-KS₆), and eryA ORFI (ACP₁-KS₁; ACP₂-KS₂), ORF2 (ACP₃-KS₄) and ORF3 (ACP₅-KS₆).

This approach can also be used to produce shorter chain fatty acid groups by limiting the ability of the FAS unit to generate long-chain fatty acids. Mutagenesis of DNA encoding various FAS catalytic activities, starting with the KS, may result in the synthesis of short-chain (R)-3-hydroxy fatty acids.

The PHA polymers are then recovered from the biomass. Large-scale solvent extraction can be used, but is expensive. An alternative method involving heat shock with subsequent enzymatic and detergent digestive processes is also available (Byron, Trends Biotechnical, 5, 246(1987); Holmes, In: Developments in Crystalline Polymers, D. C. Bassett (ed.), pp. 1-65 (1988)). PHB and other PHAs are readily extracted from microorganisms by chlorinated hydrocarbons. Refluxing with chloroform has been extensively used; the resulting solution is filtered to remove debris and concentrated, and the polymer is precipitated with methanol or ethanol, leaving low-molecular-weight lipids in solution. Longer side-chain PHAs show a less restricted solubility than PHB and are, for example, soluble in acetone. Other strategies adopted include the use of ethylene carbonate and propylene carbonate as disclosed by Lafferty et al. (Chem, Rundschau, 30, 14 (1977)) to extract PHB from biomass. Scandola et al. (Int. J. Biol. Microbiol., 10, 373 (1988)) reported that 1 M HCl-chloroform extraction of Rhizobium meliloti yielded PHB of M_{W}=6×10⁴ compared with 1.4 ×10⁶ when acetone was used.

Methods are well known in the art for the determination of the PHB or PHA content of microorganisms, the composition of PHAs, and the distribution of the monomer units in the polymer. Gas chromatography and high-pressure liquid chromatography are widely used for quantitative PHB analysis. See Anderson et al., Microbiol. Rev, 54, 450 (1990) for a review of such methods. NMR techniques can also be used to determine polymer composition, and the distribution of monomer units.

Variants of the Nucleic Acid Molecules of the Invention

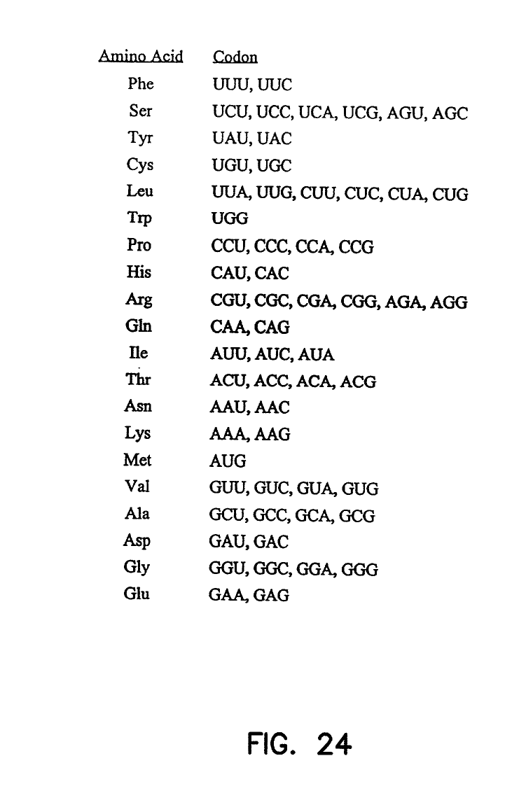

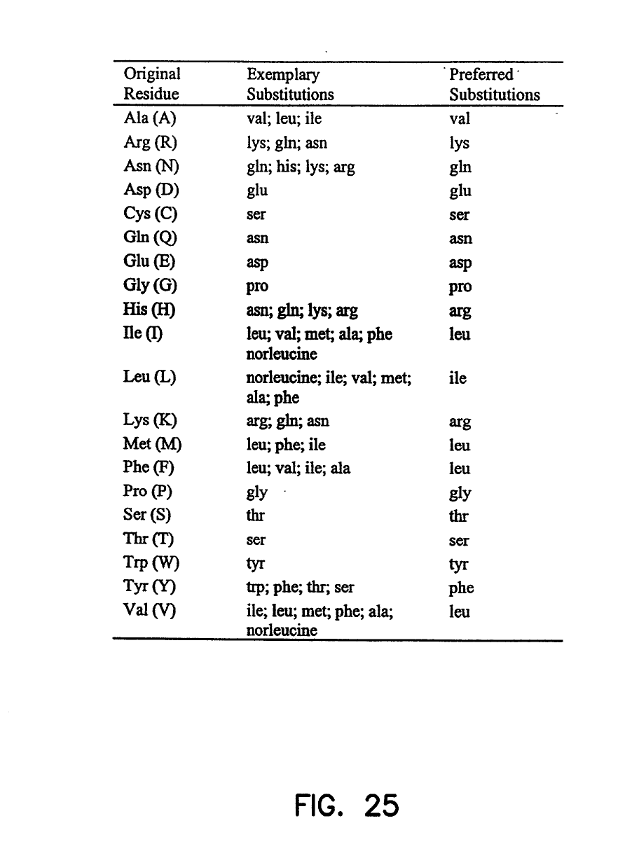

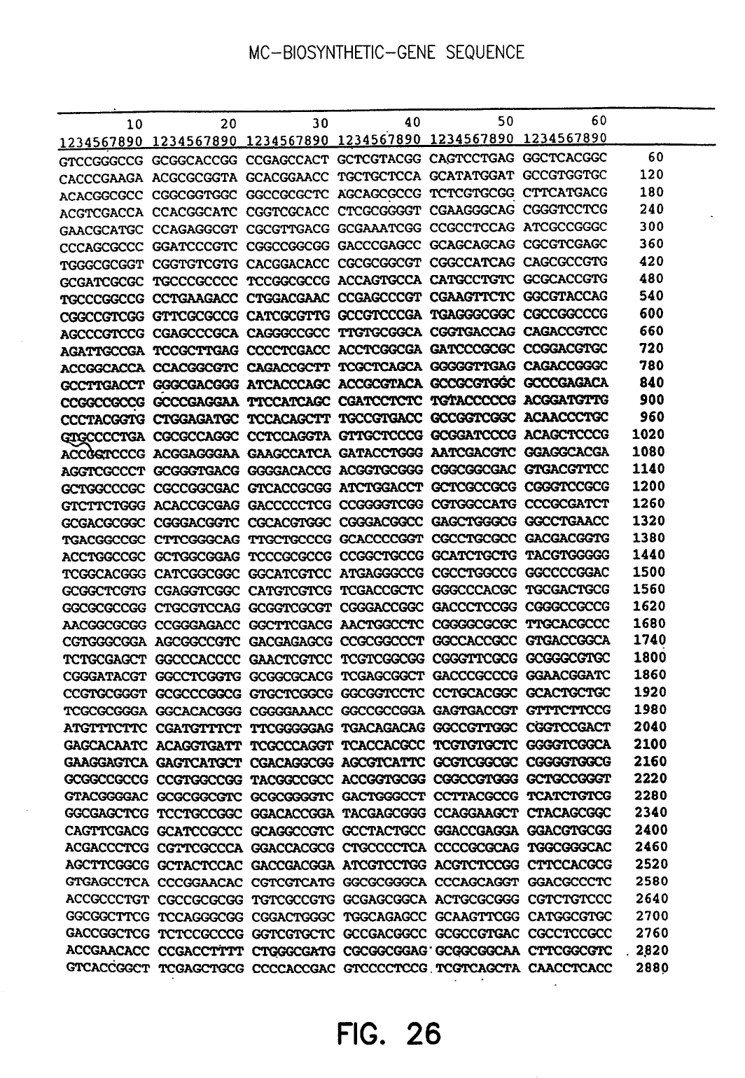

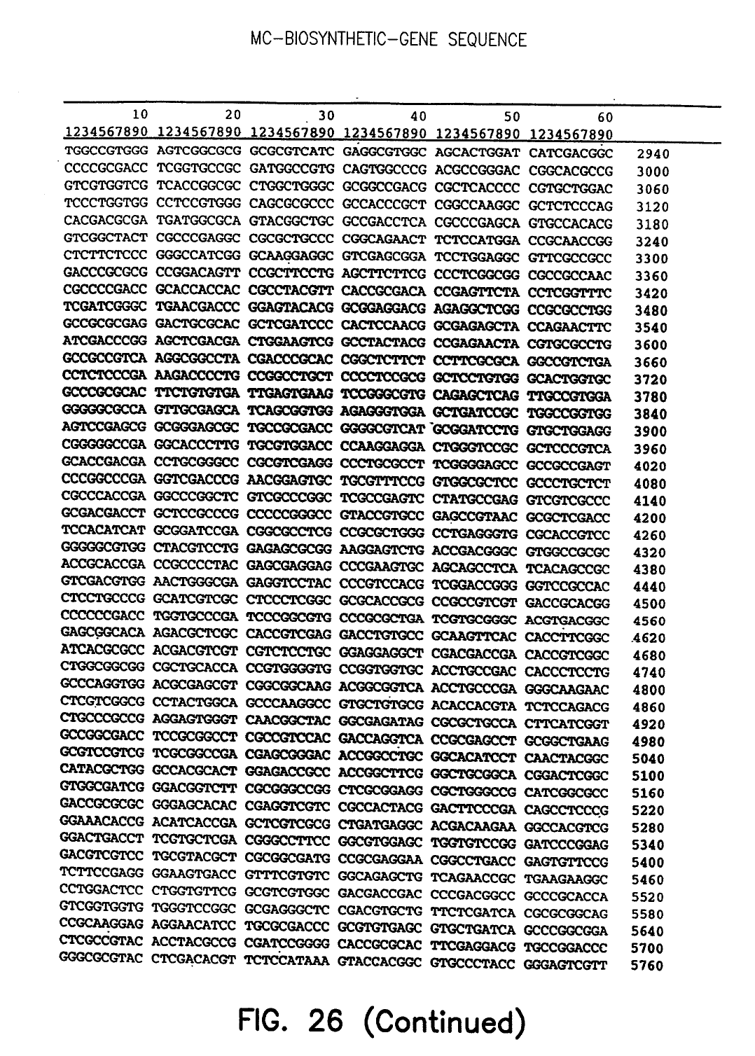

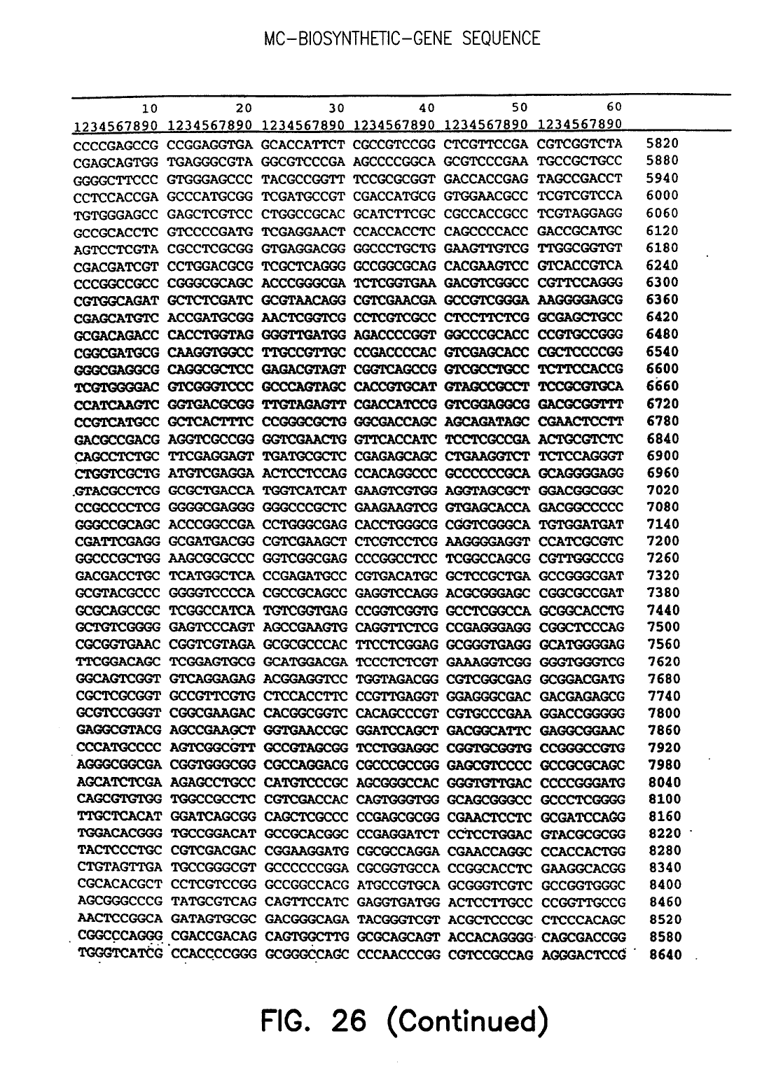

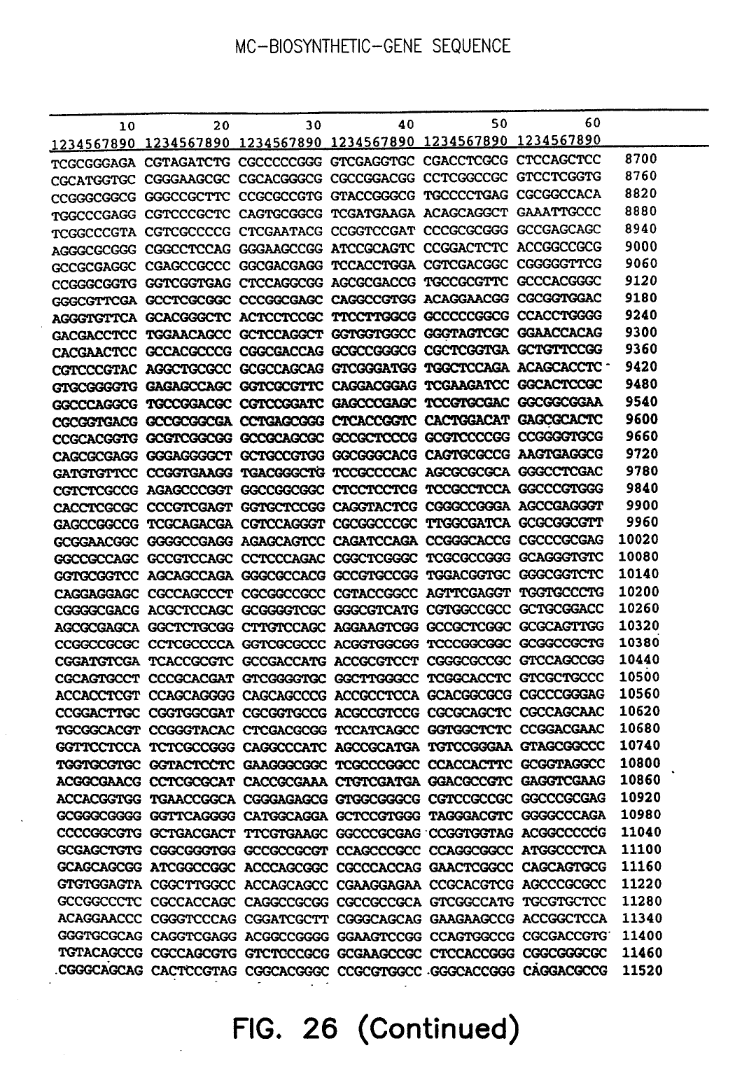

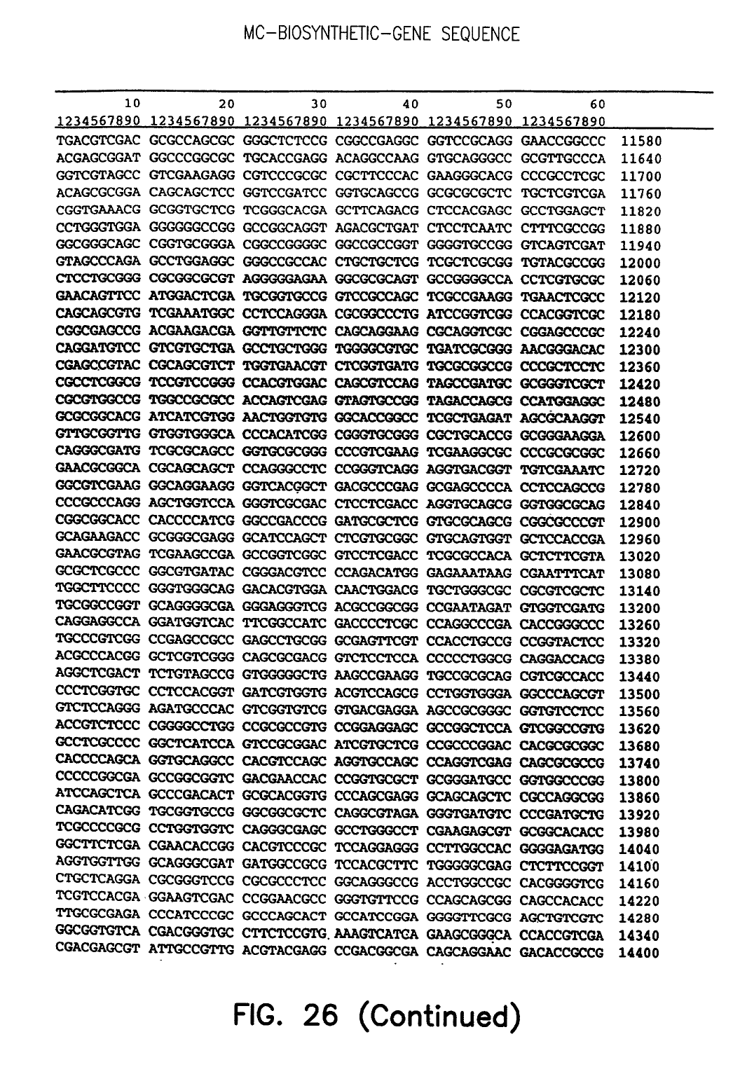

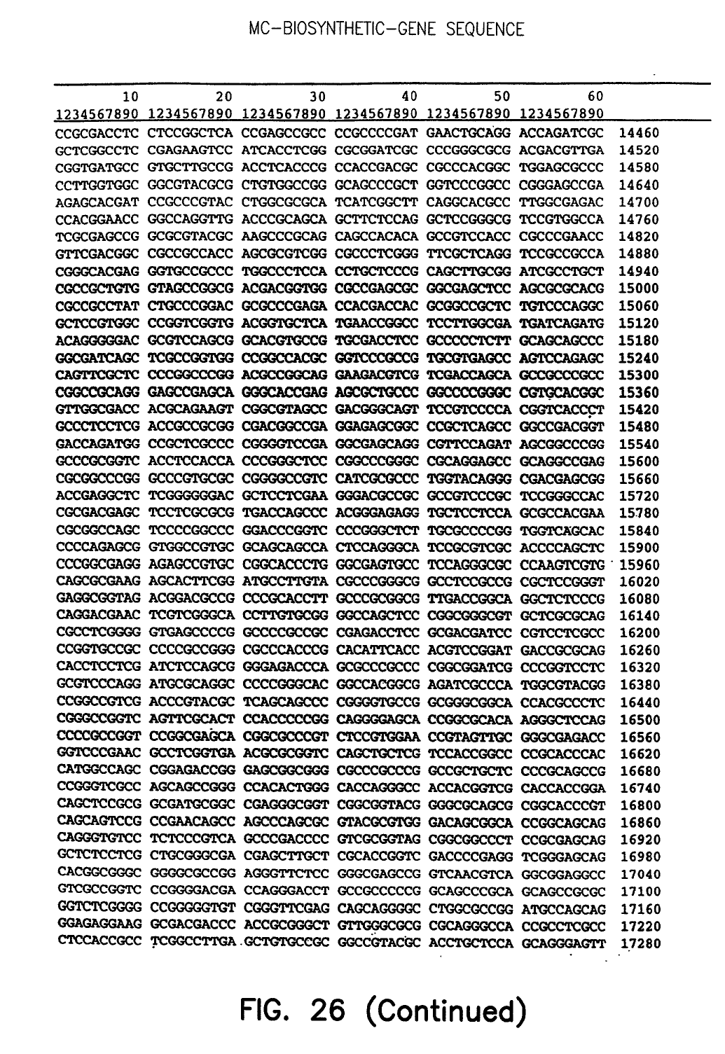

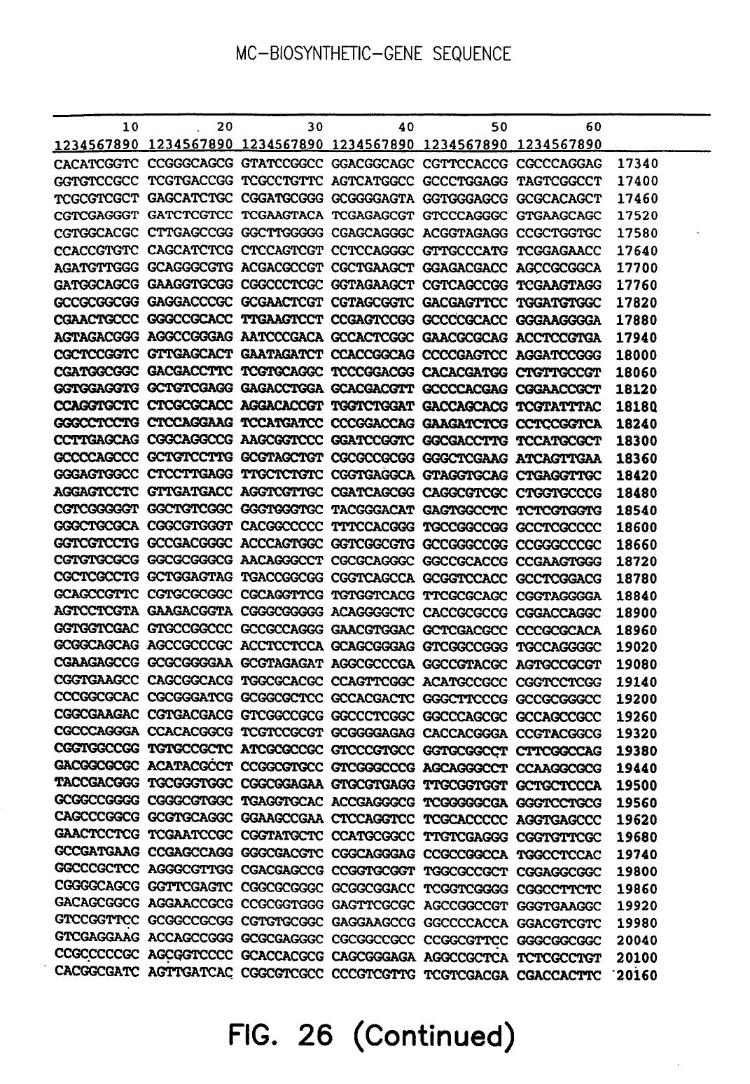

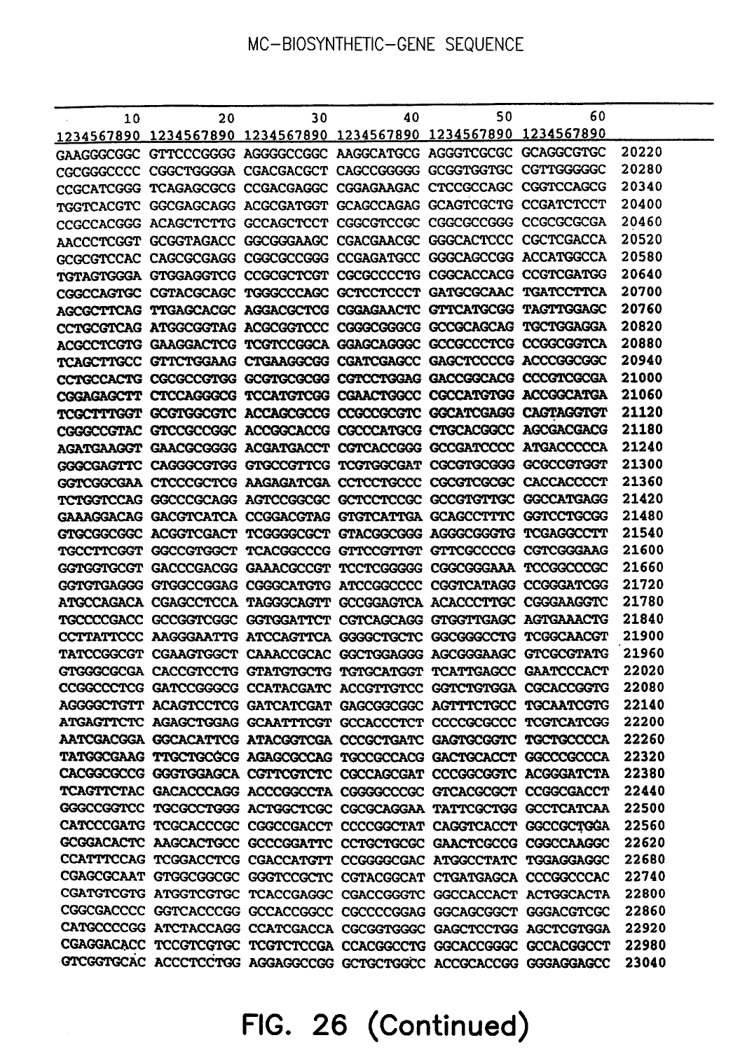

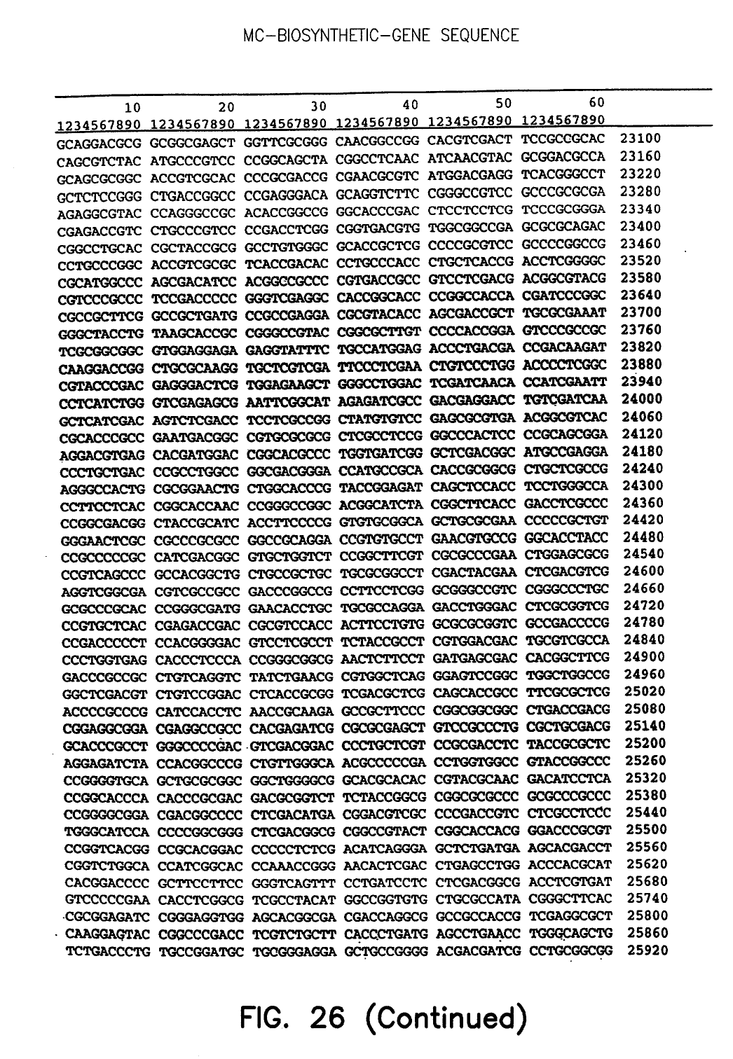

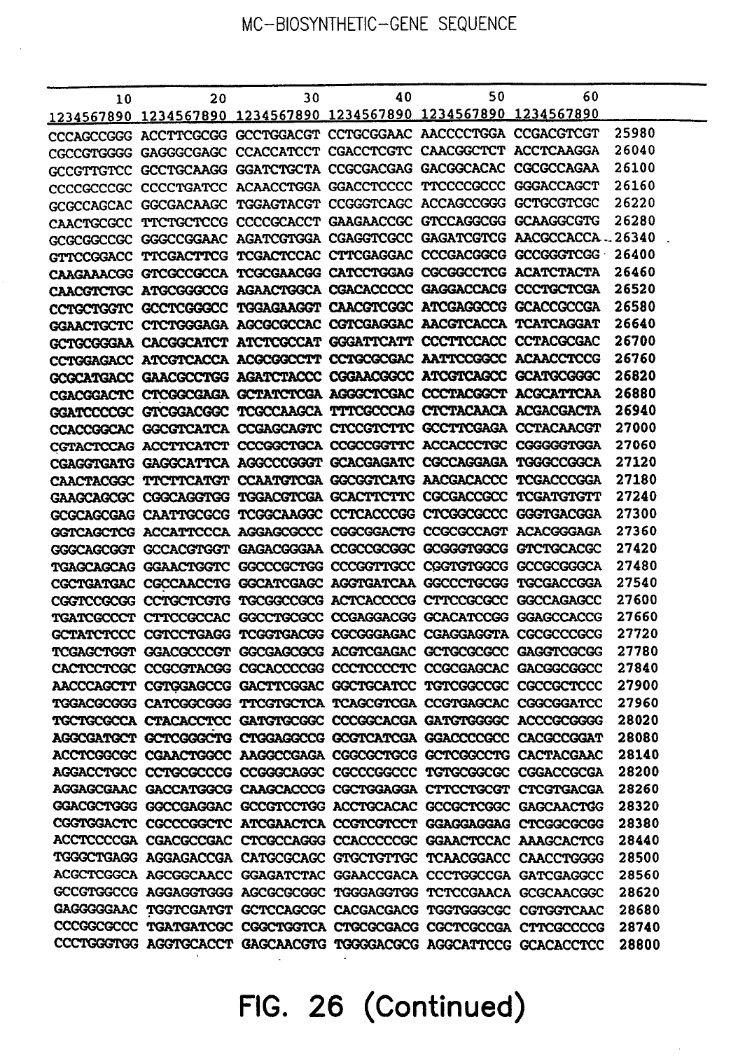

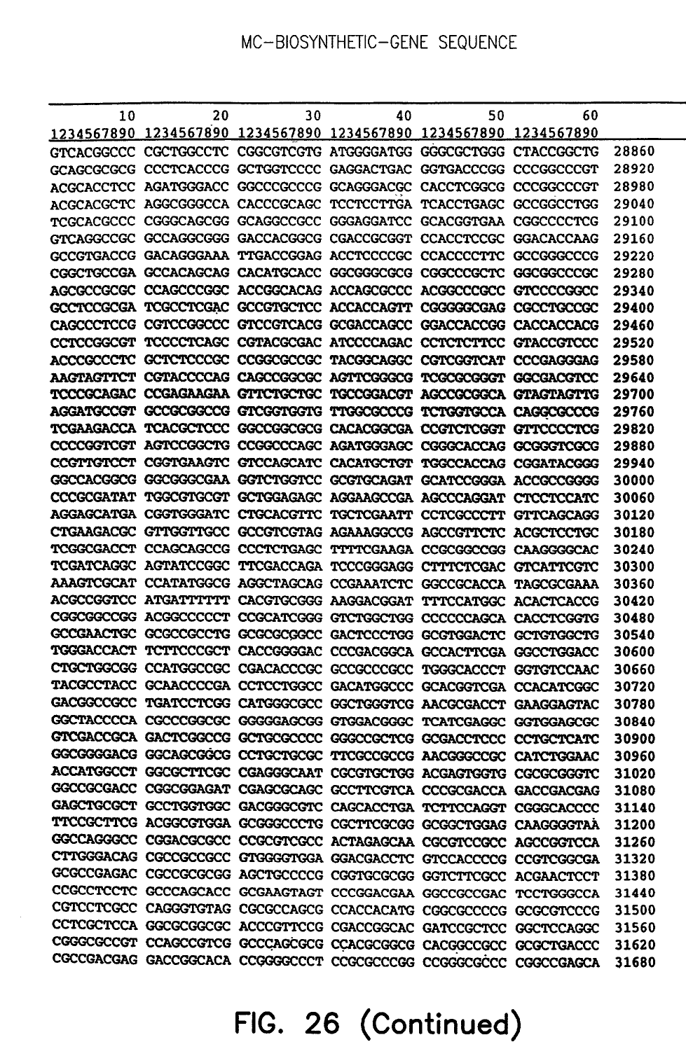

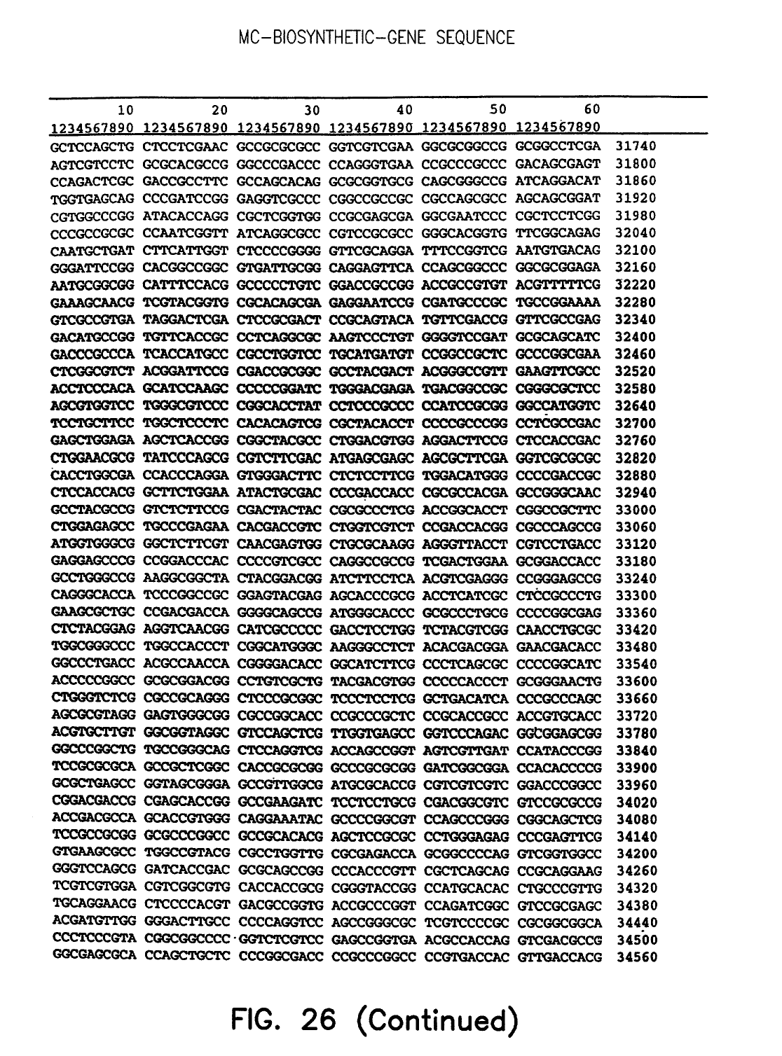

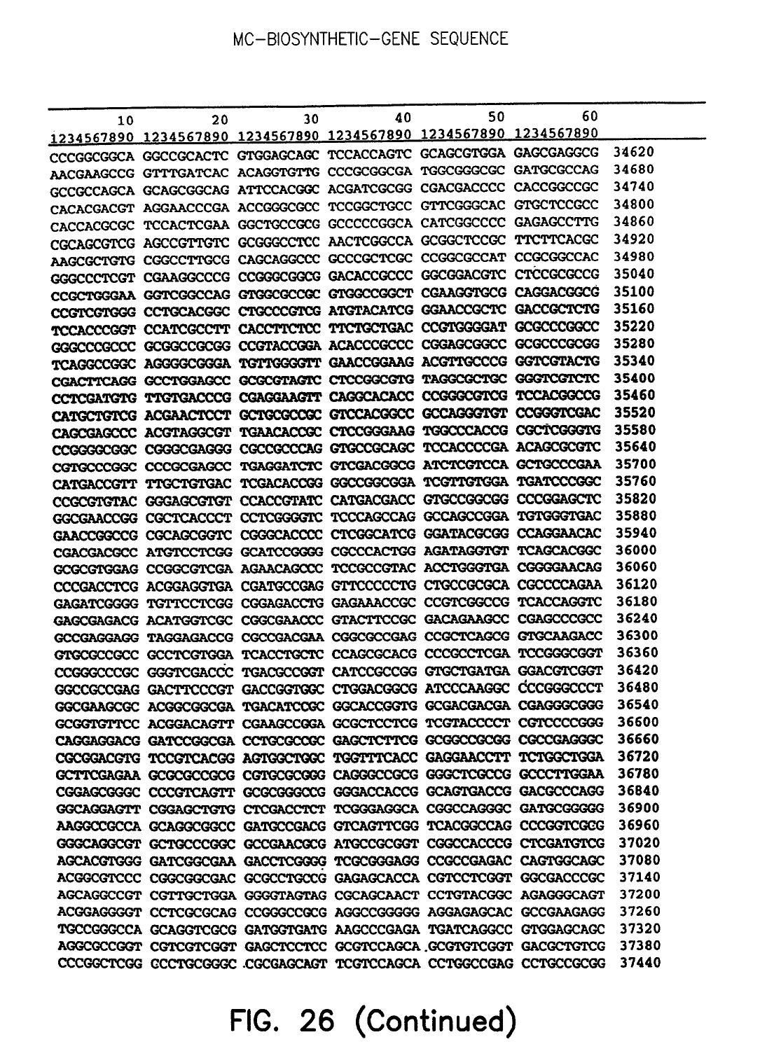

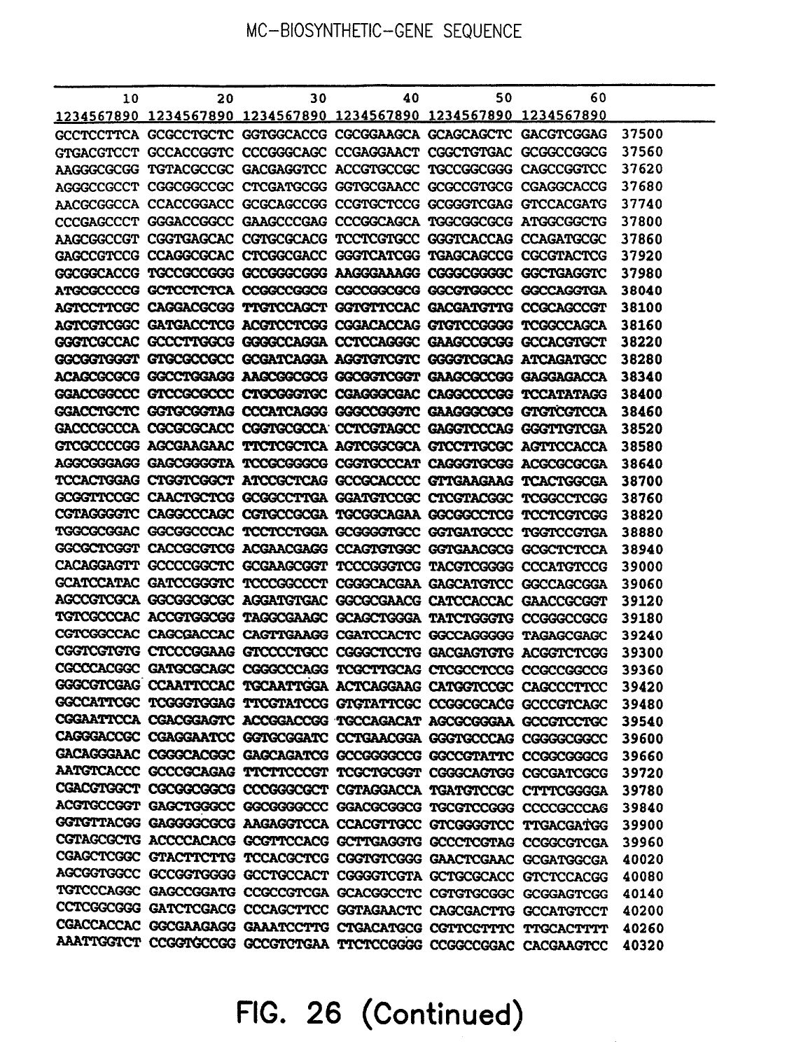

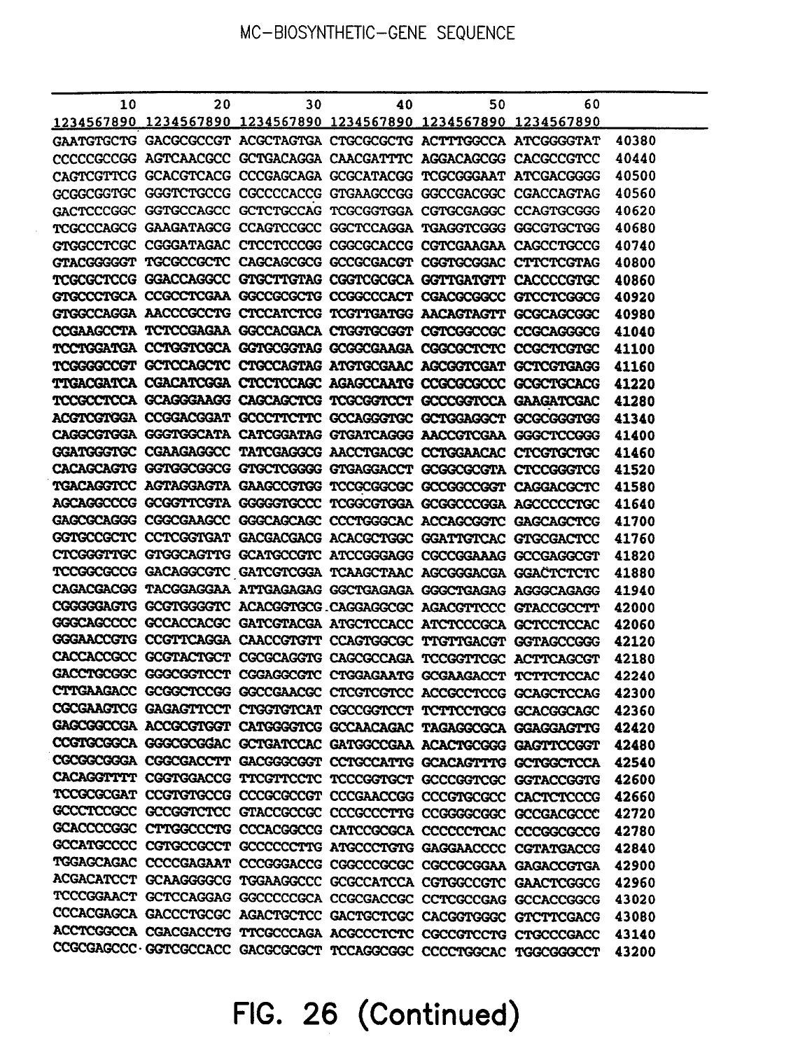

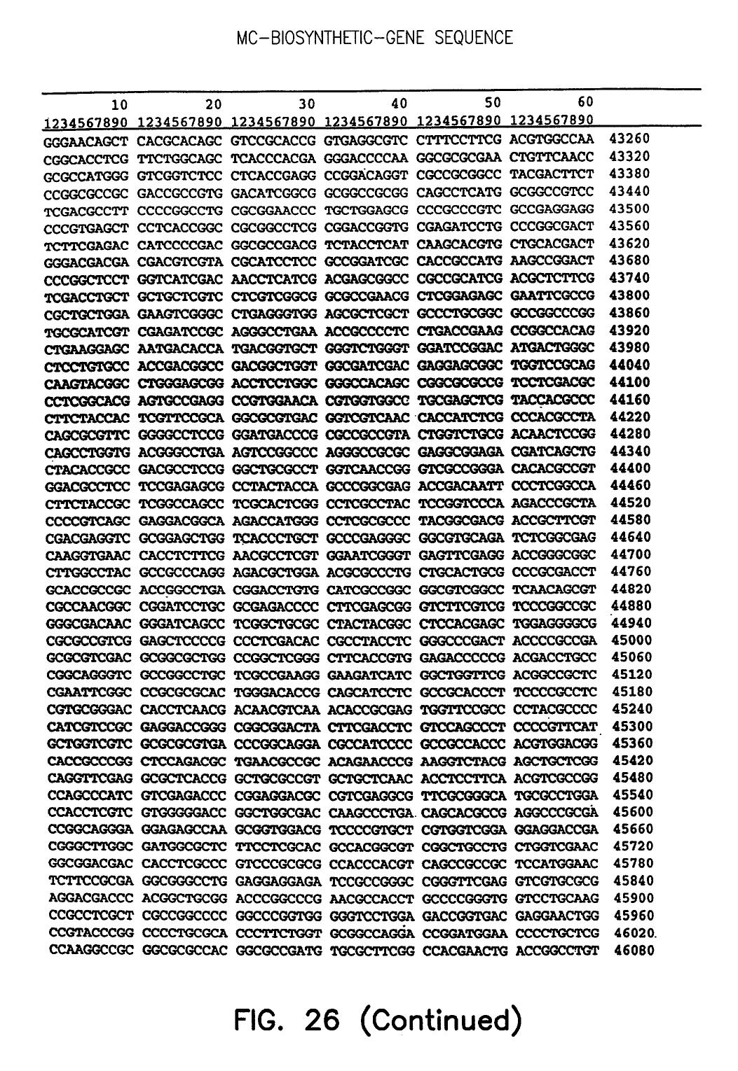

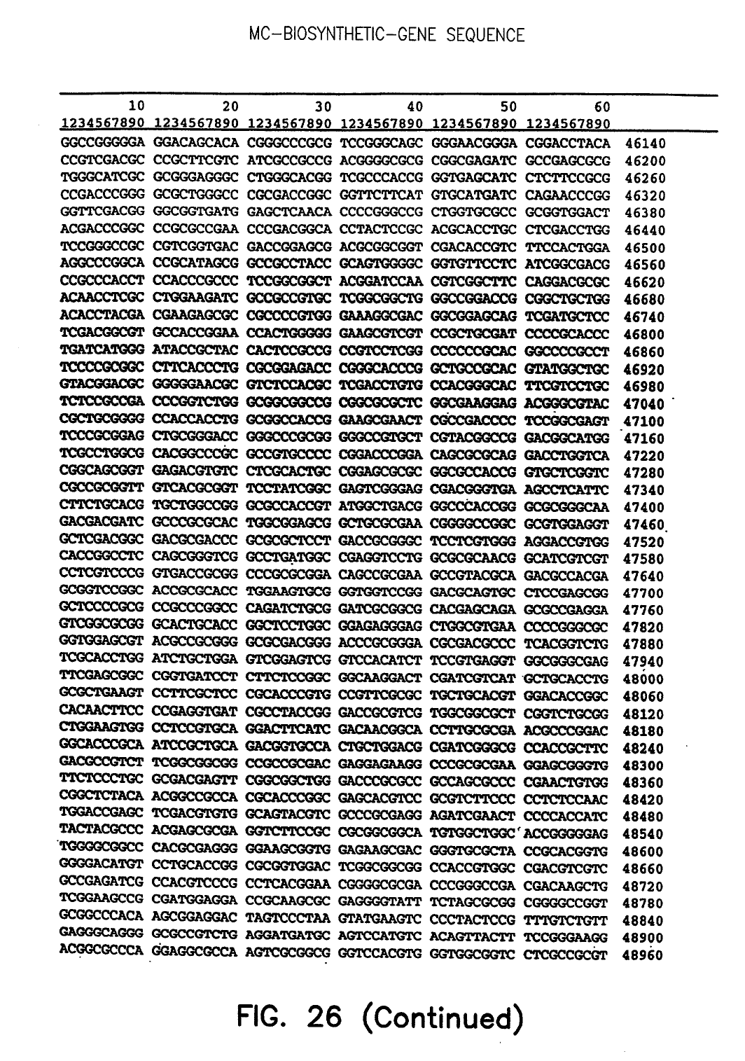

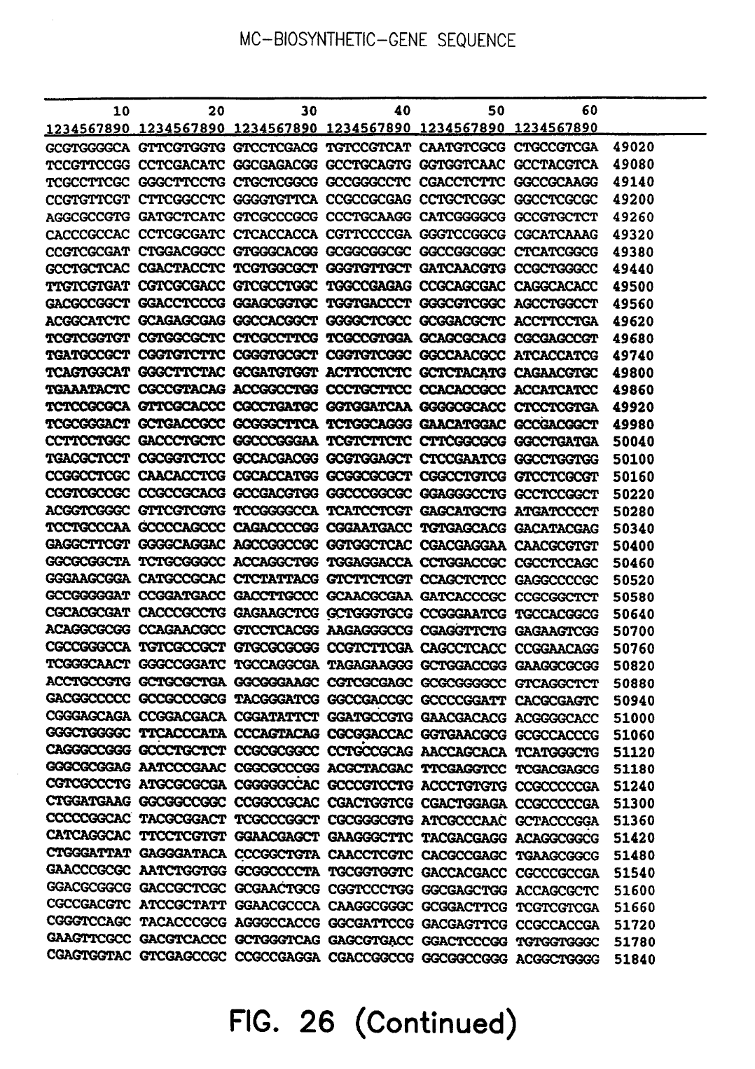

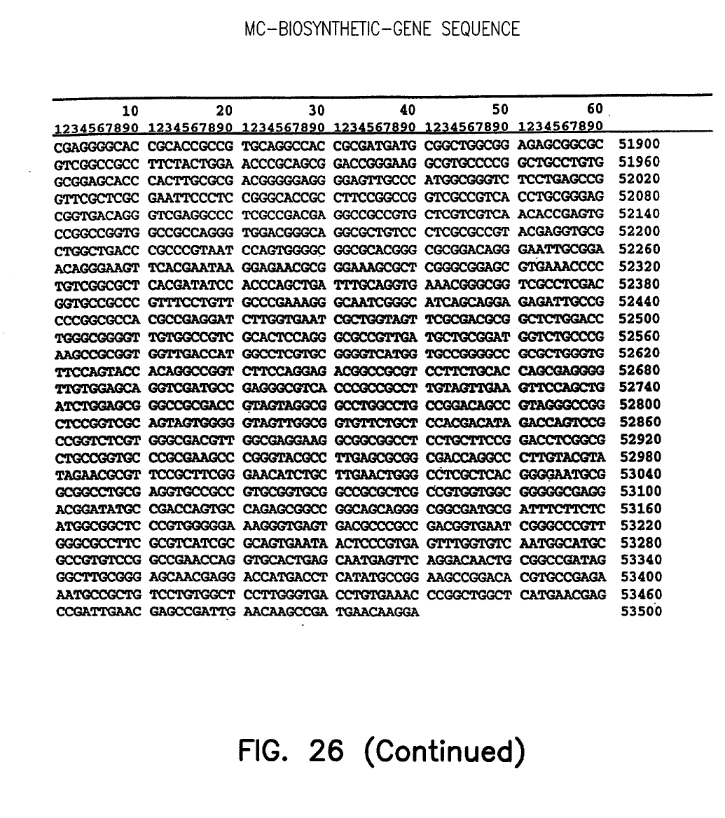

The present invention contemplates nucleic acid sequences which hybridize under low, medium or high stringency hybridization conditions to the exemplified nucleic acid sequences set forth herein. Hybridization conditions are well known in the art. Thus, nucleic acid sequences encoding variant polypeptides, i.e., those having at least one amino acid substitution insertion, addition or deletion, or nucleic acid sequences having conservative (e.g., silent) nucleotide substitutions (see FIGS. 24-25), are within the scope of the invention. Preferably, variant polypeptides encoded by the nucleic acid sequences of the invention are biologically active. The present invention also contemplates naturally occurring allelic variations and mutations of the nucleic acid sequences described herein.

As is well known in the art, because of the degeneracy of the genetic code, there are numerous other DNA and RNA molecules that can code for the same polypeptides as those encoded by the exemplified biosynthetic genes and fragments thereof The present invention, therefore, contemplates those other DNA and RNA molecules which, on expression, encode the polypeptides of, for example, portions of SEQ ID NO:96. Having identified the amino acid residue sequence encoded by a mitomycin, sugar or polyketide biosynthetic gene, and with knowledge of all triplet codons for each particular amino acid residue, it is possible to describe all such encoding RNA and DNA sequences. DNA and RNA molecules other than those specifically disclosed herein and, which molecules are characterized simply by a change in a codon for a particular amino acid, are within the scope of this invention.

The 20 common amino acids and their representative abbreviations, symbols and codons are well known in the art (see, for example, Molecular Biology of the Cell, Second Edition, B. Alberts et al., Garland Publishing Inc., New York and London, 1989). As is also well known in the art, codons constitute triplet sequences of nucleotides in mRNA molecules and as such, are characterized by the base uracil (U) in place of base thymidine (T) which is present in DNA molecules. A simple change in a codon for the same amino acid residue within a polynucleotide will not change the structure of the encoded polypeptide. By way of example, it canbe seen from SEQ ID NO:16 that a TCA codon for serine exists at nucleotide positions 146-148. However, serine can be encoded by a TCT codon, and a TCC codon. Substitution of the latter codons for serine with the TCA codon for serine or vice versa, does not substantially alter the DNA sequence of SEQ ID NO:16 and results in production of the same polypeptide. In a similar manner, substitutions of the recited codons with other equivalent codons can be made in a like manner without departing from the scope of the present invention.

A nucleic acid molecule, segment or sequence of the present invention can also be an RNA molecule, segment or sequence. An RNA molecule contemplated by the present invention corresponds to, is complementary to or hybridizes under low, medium or high stringency conditions to, any of the DNA sequences set forth herein. Exemplary and preferred RNA molecules are mRNA molecules that comprise at least one mitomycin, sugar or polyketide biosynthetic gene of this invention.

Mutations can be made to the native nucleic acid sequences of the invention and such mutants used in place of the native sequence, so long as the mutants are able to function with other sequences to collectively catalyze the synthesis of an identifiable sugar, polyketide or mitomycin. Such mutations can be made to the native sequences using conventional techniques such as by preparing synthetic oligonucleotides including the mutations and inserting the mutated sequence into the gene using restriction endonuclease digestion. (See, e.g., Kunkel, T. A. Proc. Natl. Acad, Sci, USA (1985) 82:448; Geisselsoder et al. BioTechniques (1987) 5:786.) Alternatively, the mutations can be effected using a mismatched primer (generally 10-30 nucleotides in length) which hybridizes to the native nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. Zoller and Smith, Methods Enzymol., (1983) 100:468. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al., Proc. Natl. Acad. Sci. USA (1982) 79:6409. PCR mutagenesis will also find use for effecting the desired mutations.

Random mutagenesis of the nucleotide sequence can be accomplished by several different techniques known in the art, such as by altering sequences within restriction endonuclease sites, inserting an oligonucleotide linker randomly into a plasmid, by irradiation with X-rays or ultraviolet light, by incorporating incorrect nucleotides during in vitro DNA synthesis, by error-prone PCR mutagenesis, by preparing synthetic mutants or by damaging plasmid DNA in vitro with chemicals. Chemical mutagens include, for example, sodium bisulfite, nitrous acid, hydroxylamine, agents which damage or remove bases thereby preventing normal base-pairing such as hydrazine or formic acid, analogues of nucleotide precursors such as nitrosoguanidine, 5-bromouracil, 2-aminopurine, or acridine intercalating agents such as proflavine, acriflavine, quinacrine, and the like. Generally, plasmid DNA or DNA fragments are treated with chemicals, transformed into E. coli and propagated as a pool or library of mutant plasmids.

Large populations of random enzyme variants can be constructed in vivo using “recombination-enhanced mutagenesis.” This method employs two or more pools of, for example, 10⁶ mutants each of the wild-type encoding nucleotide sequence that are generated using any convenient mutagenesis technique and then inserted into cloning vectors.

Chimeric Expression Cassettes, Vectors and Host Cells of the Invention

As used herein, “chimeric” means that a vector comprises DNA from at least two different species, or comprises DNA from the same species, which is linked or associated in a manner which does not occur in the “native” or wild type of the species. The recombinant DNA sequence or segment, used for transformation herein, may be circular or linear, double-stranded or single-stranded. Generally, the DNA sequence or segment is in the form of chimeric DNA, such as plasmid DNA, that can also contain coding regions flanked by control sequences which promote the expression of the DNA present in the resultant transformed (recombinant) host cell. Aside from DNA sequences that serve as transcription units for the nucleic acid molecules of the invention or portions thereof, a portion of the DNA may be untranscribed, serving a regulatory or a structural function. For example, the preselected DNA may itself comprise a promoter that is active in a particular host cell.

Other elements functional in the host cells, such as introns, enhancers, polyadenylation sequences and the like, may also be a part of the DNA. Such elements may or may not be necessary for the function of the DNA, but may provide improved expression of the DNA by affecting transcription, stability of the MRNA, or the like. Such elements may be included in the DNA as desired to obtain the optimal performance of the transforming DNA in the cell.

“Control sequences” is defined to mean DNA sequences necessary for the expression of an operably linked coding sequence in a particular host organism. The control sequences that are suitable for prokaryotic cells, for example, include a promoter, and optionally an operator sequence, and a ribosome binding site. Eukaryotic cells are known to utilize promoters, polyadenylation signals, and enhancers. Other regulatory sequences may also be desirable which allow for regulation of expression of the genes relative to the growth of the host cell. Regulatory sequences are known to those of skill in the art, and examples include those which cause the expression of a gene to be turned on or off in response to a chemical or physical stimulus, including the presence of a regulatory compound. Othertypes of regulatory elements may also be present in the vector, for example, enhancer sequences.

“Operably linked” is defined to mean that the nucleic acids are placed in a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accord with conventional practice.

The DNA to be introduced into the cells further will generally contain either a selectable marker gene or a reporter gene or both to facilitate identification and selection of transformed cells from the population of cells sought to be transformed. Alternatively, the selectable marker may be carried on a separate piece of DNA and used in a co-transformation procedure. Both selectable markers and reporter genes may be flanked with appropriate regulatory sequences to enable expression in the host cells. Useful selectable markers are well known in the art and include, for example, antibiotic and herbicide-resistance genes, such as neo, hpt, dhfr, bar, aroa, dapa and the like. See also, the genes listed on Table 1 of Lundquist et al. (U.S. Pat. No. 5,848,956).

Reporter genes are used for identifying potentially transformed cells and for evaluating the functionality of regulatory sequences. Reporter genes which encode for easily assayable proteins are well known in the art. In general, a reporter gene is a gene which is not present in or expressed by the recipient organism or tissue and which encodes a protein whose expression is manifested by some easily detectable property, e.g., enzymatic activity. Expression of the reporter gene is assayed at a suitable time after the DNA has been introduced into the recipient cells.

Prokaryotic expression systems are preferred, and in particular, systems compatible with Streptomyces spp. are of particular interest. Control elements for use in such systems include promoters, optionally containing operator sequences, and ribosome binding sites. Particularly useful promoters include control sequences derived from the gene clusters of the invention. However, other bacterial promoters, such as those derived from sugar metabolizing enzymes, such as galactose, lactose (lac) and maltose, will also find use in the expression cassettes encoding desosamine. Preferred promoters are Streptomyces promoters, including but not limited to the ermE*, pita and tipA promoters. Additional examples include promoter sequences derived from biosynthetic enzymes such as tryptophan (trp), the β-lactamase (bla) promoter system, bacteriophage lambda PL, and T5. In addition, synthetic promoters, such as the tac promoter (U.S. Pat. No 4,551,433), which do not occur in nature, also fimction in bacterial host cells.

The various nucleic acid molecules of interest can be cloned into one or more recombinant vectors as individual cassettes, with separate control elements, or under the control of, e.g., a single promoter. The nucleic acid molecules can include flanking restriction sites to allow for the easy deletion and insertion of other sequences. The design of such unique restriction sites is known to those of skill in the art and can be accomplished using the techniques, such as site-directed mutagenesis and PCR.

For sequences generated by random mutagenesis, the choice of vector depends on the pool of mutant sequences, i.e., donor or recipient, with which they are to be employed. Furthermore, the choice of vector determines the host cell to be employed in subsequent steps of the claimed method. Any transducible cloning vector can be used as a cloning vector for the donor pool of mutants. It is preferred, however, that phagemids, cosmids, or similar cloning vectors be used for cloning the donor pool of mutant encoding nucleotide sequences into the host cell. Phagemids and cosmids, for example, are advantageous vectors due to the ability to insert and stably propagate therein larger fragments of DNA than in M13 phage and λ phage, respectively. Phagemids which will find use in this method generally include hybrids between plasmids and filamentous phage cloning vehicles. Cosmids which will find use in this method generally include λ phage-based vectors into which cos sites have been inserted. Recipient pool cloning vectors can be any suitable plasmid. The cloning vectors into which pools of mutants are inserted may be identical or may be constructed to harbor and express different genetic markers (see, e.g., Sambrook et al., supra). The utility of employing such vectors having different marker genes may be exploited to facilitate a determination of successful transduction.

Thus, for example, the cloning vector employed may be an E. coli/Streptomyces shuttle vector (see, for example, U.S. Pat. Nos. 4,416,994, 4,343,906,4,477,571, 4,362,816, and 4,340,674), a cosmid, a plasmid, an artificial bacterial chromosome (see, e.g., Zhang and Wing, Plant Mol. Biol, 35, 115 (1997); Schalkwyk et al., Curr Op. Biotech., 6, 37 91995); and Monaco and Lavin, Trends in Biotech., 12, 280 (1994), or a phagemid, and the host cell may be a bacterial cell such as E. coli, Penicillium patulum, and Streptomyces spp. such as S. lividans, S. venezuelae, or S. lavendulae, or a eukaryotic cell such as fungi, yeast or a plant cell, e.g., monocot and dicot cells, preferably cells that are regenerable.

The general methods for constructing recombinant DNA which can transform target cells are well known to those skilled in the art, and the same compositions and methods of construction may be utilized to produce the DNA useful herein. For example, J. Sambrook et al., Molecular Cloning: A Laboratory Manual, Cold Spring Harbor Laboratory Press (2d ed., 1989), provides suitable methods of construction.

The recombinant DNA can be readily introduced into the host cells by any procedure useful for the introduction into a particular cell, e.g., calcium phosphate precipitation, protoplast fusion, conjugation, lipofection, electroporation, and the like.

As used herein, the term “cell line” or “host cell” is intended to refer to well-characterized homogenous, biologically pure populations of cells. These cells may be eukaryotic cells that are neoplastic or which have been “immortalized” in vitro by methods known in the art, as well as primary cells, or prokaryotic cells. In particular, the cell line or host cell may be of mammalian, plant, insect, yeast, fungal or bacterial origin.

“Transfected” or “transformed” is used herein to include any host cell or cell line, the genome of which has been altered or augmented by the presence of at least one DNA sequence, which DNA is also referred to in the art of genetic engineering as “heterologous DNA,” “recombinant DNA,” “exogenous DNA,” “genetically engineered,” “non-native,” or “foreign DNA,” wherein said DNA was isolated and introduced into the genome of the host cell or cell line by the process of genetic engineering. The transfected DNA may be maintained as an extrachromosomal element or as an element which is stably integrated into the host chromosome.

Moreover, recombinant polypeptides having a particular activity may be prepared via “gene-shuffling”. See, for example, Crameri et al., Nature, 391, 288(1998); Patten et al., Curr. Op. Biotech., 8, 724(1997), U.S. Pat. Nos. 5,837,458, 5,834,252, 5,830,727, 5,811,238, 5,605,793).

For phagemids, upon infection of the host cell which contains a phagemid, single-stranded phagemid DNA is produced, packaged and extruded from the cell in the form of a transducing phage in a manner similar to other phage vectors. Thus, clonal amplification of mutant encoding nucleotide sequences carried by phagemids is accomplished by propagating the phagemids in a suitable host cell.

Following clonal amplification, the cloned donor pool of mutants is infected with a helper phage to obtain a mixture of phage particles containing either the helper phage genome or phagemids mutant alleles of the wild-type encoding nucleotide sequence.

Infection, or transfection, of host cells with helper phage is generally accomplished by methods well known in the art (see., e.g., Sambrook et al., supra; and Russell et al. (1986) Gene 4:333-338).

The helper phage may be any phage which can be used in combination with the cloning phage to produce an infective transducing phage. For example, if the cloning vector is a cosmid, the helper phage will necessarily be a λ phage. Preferably, the cloning vector is a phagemid and the helper phage is a filamentous phage, and preferably phage M13.

If desired after infecting the phagemid with helper phage and obtaining a mixture of phage particles, the transducing phage can be separated from helper phage based on size difference (Barnes et al. (1983) Methods Enzymol. 1(1:98-122), or other similarly effective technique.

The entire spectrum of cloned donor mutations can now be transduced into clonally amplified recipient cells into which has been transduced or transformed a pool of mutant encoding nucleotide sequences. Recipient cells which may be employed in the method disclosed and claimed herein may be, for example, E. coli, or other bacterial expression systems which are not recombination deficient. A recombination deficient cell is a cell in which recombinatorial events is greatly reduced, such as rec mutants of E. coli (see, Clark et al. (1965) Proc. Natl. Acad. Sci. USA 53:451-459).

These transductants can now be selected for the desired expressed protein property or characteristic and, if necessary or desirable, amplified. Optionally, if the phagemids into which each pool of mutants is cloned are constructed to express different genetic markers, as described above, transductants may be selected by way of their expression of both donor and recipient plasmid markers

The recombinants generated by the above-described methods can then be subjected to selection or screening by any appropriate method, for example, enzymatic or other biological activity.

The above cycle of amplification, infection, transduction, and recombination may be repeated any number of times using additional donor pools cloned on phagemids. As above, the phagemids into which each pool of mutants is cloned may be constructed to express a different marker gene. Each cycle could increase the number of distinct mutants by up to a factor of 10⁶. Thus, if the probability of occurrence of an inter-allelic recombination event in any individual cell is f (a parameter that is actually a function of the distance between the recombining mutations), the transduced culture from two pools of 10⁶ allelic mutants will express up to 10¹² distinct mutants in a population of 10¹²/fcells.

Preparation, Isolation and Modification of the Polypeptides of the Invention

The present isolated, purified polypeptides, variants or fragments thereof, can be synthesized in vitro, e.g., by the solid phase peptide synthetic method or by recombinant DNA approaches (see above). The solid phase peptide synthetic method is an established and widely used method, which is described in the following references: Stewart et al., Solid Phase Peptide Synthesis, W. H. Freeman Co., San Francisco (1969); Merrifield, J. Am. Chem. Soc., 85 2149 (1963); Meienhofer in “Hormonal Proteins and Peptides,” ed.; C. H. Li, Vol. 2 (Academic Press, 1973), pp. 48-267; Bavaay and Merrifield, “The Peptides,” eds. E. Gross and F. Meienhofer, Vol. 2 (Academic Press, 1980) pp. 3-285; and Clark-Lewis et al., Meth. Enzymol, 287, 233 (1997). These polypeptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; or ligand affinity chromatography.

In particular, fusion polypeptides are prepared which comprise an amino acid sequence useful in purification, e.g., a His tag is useful to purify fusion polypeptides on nickel columns. Once isolated and characterized, derivatives, e.g., chemically derived derivatives, of a given polypeptide can be readily prepared. For example, amides of the polypeptides of the present invention may also be prepared by techniques well known in the art for converting a carboxylic acid group or precursor, to an amide. A preferred method for amide formation at the C-terminal carboxyl group is to cleave the polypeptide from a solid support with an appropriate amine, or to cleave in the presence of an alcohol, yielding an ester, followed by aminolysis with the desired amine.

Salts of carboxyl groups of a polypeptide or polypeptide variant of the invention may be prepared in the usual manner by contacting the polypeptide with one or more equivalents of a desired base such as, for example, a metallic hydroxide base, e.g., sodium hydroxide; a metal carbonate or bicarbonate base such as, for example, sodium carbonate or sodium bicarbonate; or an amine base such as, for example, triethylamine, triethanolamine, and the like.

N-acyl derivatives of an amino group of the polypeptide or polypeptide variants may be prepared by utilizing an N-acyl protected amino acid for the final condensation, or by acylating a protected or unprotected polypeptide. O-acyl derivatives may be prepared, for example, by acylation of a free hydroxy peptide or peptide resin. Either acylation may be carried out using standard acylating reagents such as acyl halides, anhydrides, acyl imidazoles, and the like. Both N- and O-acylation may be carried out together, if desired.

One or more of the residues of the polypeptide can be altered, so long as the polypeptide variant is biologically active. For example, it is preferred that the variant has at least about 1% of the biological activity of the corresponding non-variant polypeptide, e.g. Conservative amino acid substitutions are preferred—that is, for example, aspartic-glutamic as acidic amino acids; lysinelarginine/histidine as basic amino acids; leucine/isoleucine, methionine/valine, alanine/valine as hydrophobic amino acids; serine/glycine/alanine/thereonine as hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfurontaining side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional polypeptide can readily be determined by assaying the specific activity of the polypeptide variant.

Conservative substitutions are shown in FIG. 25 under the heading of exemplary substitutions. More preferred substitutions are under the heading of preferred substitutions. After the substitutions are introduced, the variants are screened for biological activity.

Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic; trp, tyr, phe.

The invention also envisions polypeptide variants with non-conservative substitutions. Non-conservative substitutions entail exchanging a member of one of the classes described above for another.

Acid addition salts of the polypeptide or variant polypeptide or of amino residues of the polypeptide or variant polypeptide may be prepared by contacting the polypeptide or amine with one or more equivalents of the desired inorganic or organic acid, such as, for example, hydrochloric acid. Esters of carboxyl groups of the polypeptides may also be prepared by any of the usual methods known in the art.

Antibodies of the Invention

The antibodies of the invention are prepared by using standard techniques. To prepare polyclonal antibodies or “antisera,” an animal is inoculated with an antigen that is an isolated and purified polypeptide of the invention, and immunoglobulins are recovered from a fluid, such as blood serum, that contains the immunoglobulins, after the animal has had an immune response. For inoculation, the antigen is preferably bound to a carrier peptide and emulsified using a biologically suitable emulsifying agent, such as Freund's incomplete adjuvant. A variety of mammalian or avian host organisms may be used to prepare polyclonal antibodies

Following immunization, Ig is purified from the immunized bird or mammal, e.g., goat, rabbit, mouse, rat, or donkey and the like. For certain applications, it is preferable to obtain a composition in which the antibodies are essentially free of antibodies that do not react with the immunogen. This composition is composed virtually entirely of the high titer, monospecific, purified polyclonal antibodies to the antigen. Antibodies can be purified by affinity chromatography. Purification of antibodies by affinity chromatography is generally known to those skilled in the art (see, for example, U.S. Pat. No. 4,533,630). Briefly, the purified antibody is contacted with the purified polypeptide, or a peptide thereof, bound to a solid support for a sufficient time and under appropriate conditions for the antibody to bind to the polypeptide or peptide. Such time and conditions are readily determinable by those skilled in the art. The unbound, unreacted antibody is then removed, such as by washing. The bound antibody is then recovered from the column by eluting the antibodies, so as to yield purified, monospecific polyclonal antibodies.

Monoclonal antibodies can be also prepared, using known hybridoma cell culture techniques. In general, this method involves preparing an antibody-producing fused cell line, e.g., of primary spleen cells fused with a compatible continuous line of myeloma cells, and growing the fused cells either in mass culture or in an animal species, such as a murine species, from which the myeloma cell line used was derived or is compatible. Such antibodies offer many advantages in comparison to those produced by inoculation of animals, as they are highly specific and sensitive and relatively “pure” immunochemically. Immunologically active fragments of the present antibodies are also within the scope of the present invention, e.g., the F(ab) fragment, scFv antibodies, as are partially humanized monoclonal antibodies.

Thus, it will be understood by those skilled in the art that the hybridomas herein referred to may be subject to genetic mutation or other changes while still retaining the ability to produce monoclonal antibody of the same desired specificity. The present invention encompasses mutants, other derivatives and descendants of the hybridomas.

It will be further understood by those skilled in the art that a monoclonal antibody may be subjected to the techniques of recombinant DNA technology to produce other derivative antibodies, humanized or chimeric molecules or antibody fragments which retain the specificity of the original monoclonal antibody. Such techniques may involve combining DNA encoding the immunoglobulin variable region, or the complementarity determining regions (CDRs), of the monoclonal antibody with DNA coding the constant regions, or constant regions plus framework regions, of a different immunoglobulin, for example, to convert a mouse-derived monoclonal antibody into one having largely human immunoglobulin characteristics (see EP 184187A, 2188638A, herein incorporated by reference).

The antibodies of the invention are useful for detecting or determining the presence or amount of a polypeptide of the invention in a sample. The antibodies are contacted with the sample for a period of time and under conditions sufficient for antibodies to bind to the polypeptide so as to form a binary complex between at least a portion of said antibodies and said polypeptide. Such times, conditions and reaction media can be readily determined by persons skilled in the art.

For example, the cells are lysed to yield an extract which comprises cellular proteins. Alternatively, intact cells are permneabilized in a manner which permits macromolecules, i.e., antibodies, to enter the cell. The antibodies of the invention are then incubated with the protein extract, e.g., in a Western blot, or permeabilized cells, e.g., prior to flow cytometry, so as to form a complex. The presence or amount of the complex is then determined or detected.

The antibodies of the invention may also be coupled to an insoluble or soluble substrate. Soluble substrates include proteins such as bovine serum albumin. Preferably, the antibodies are bound to an insoluble substrate, i.e., a solid support. The antibodies are bound to the support in an amount and manner that allows the antibodies to bind the polypeptide (ligand). The amount of the antibodies used relative to a given substrate depends upon the particular antibody being used, the particular substrate, and the binding efficiency of the antibody to the ligand. The antibodies may be bound to the substrate in any suitable manner. Covalent, noncovalent, or ionic binding may be used. Covalent bonding can be accomplished by attaching the antibodies to reactive groups on the substrate directly or through a linking moiety.

The solid support may be any insoluble material to which the antibodies can be bound and which may be conveniently used in an assay of the invention. Such solid supports include permeable and semipermeable membranes, glass beads, plastic beads, latex beads, plastic microtiter wells or tubes, agarose or dextran particles, sepharose, and diatomaceous earth. Alternatively, the antibodies may be bound to any porous or liquid permeable material, such as a fibrous (paper, felt etc.) strip or sheet, or a screen or net. A binder may be used as long as it does not interfere with the ability of the antibodies to bind the ligands.

The invention will be further described by the following examples.

### EXAMPLE 1

### Molecular Characterization and Analysis of the mit/mmc Biosynthetic Gene Cluster

Materials and Methods

Bacterial Strains and Cloning Vectors

S. lavendulae NRRL 2564 was used as the source strain for cosmid library construction and the creation of gene disruption mutants. E. coli DH5α was used as the host strain for constructing the library and subsequent DNA manipulation. E. coli strain S17-1 (Mazodier et al., 1989) served as the conjugative host for introducing foreign DNA into S. lavendulae. The cosmid library was constructed with the E. coli/Streptomyces shuttle vector pNJ1 (Tuan et al., 1990), and pUC119 was routinely used as a vector for subcloning and sequencing. The conjugative E. coli/Streptomyces shuttle vector pKC1139 (Bierman et al., 1992) was used for gene disruption in S. lavendulae.

DNA Manipulation

Standard in vitro techniques were used for DNA manipulation (Sambrook et al., 1989). S. lavendulae genomic DNA was harvested by standard procedures (Hopwood et al., 1985).

A library of size-fractionated genomic DNA in pNJ1 (Tuan et al., 1990) was screened with the rifamycin AHBA synthase (rifK) gene probe from Amycolatopsis mediterranei (Kim et al., 1998). Through subsequent cosmid walking, a contiguous 120 kb region of S. lavendulae chromosomal DNA containing the putative mitomycin biosynthetic genes was mapped. M13 forward and reverse primers were used for sequencing (Gibco BRL, Gaithersburg, Md.). To accomplish this, individual fragments of less than 5 kb were subcloned into pUC119 and serial deletion subclones were generated using the exonuclease III Erase-a Base System (Promega, Madison, Wis.).

DNA Sequencing and Analysis.

Automatic DNA sequencing was done with the ABI PRISM™ Dye Terminator Cycle Sequencing Ready Reaction Kit (Applied Biosystems, Warrington, U.K.), and analyzed on an Applied Biosystems mode 377 DNA Sequencer at the University of Minnesota Advanced Genetic Analysis Center. Both DNA strands were sequenced redundantly a minimum of three times. Sequence compilation was performed with MacVector (Oxford Molecular Group, Mountain View, Calif.) and GeneWorks (Oxford Molecular Group) software, and sequence homology analysis was accomplished with Blast (Altschul et al., 1990) and GCG programs (Devereux et al., 1984).

Disruption Mutants Constrcion

A 1.4 kb ApaL1-HindIII fragment from pFD666 (Denis and Brzezinski, 1998) containing the aphil gene for kanamycin resistance was routinely used as the selection marker for the creation of gene disruption constructs. The target genes were subcloned into pUC119, cut at a unique internal restriction site, blunt-ended, and ligated with the end-blunted selection marker. Tie inserts were then transferred from pUC119 to pKC1139, and conjugated into wild-type S. lavendulae. Transconjugants were selected on AS1 plates (Baltz, 1980), overlaid with apramycin, kanamycin, and nalidixic acid followed by propagation on R5T plates (g/L: sucrose 121.1, K₂SO₄ 0.3, MgCl₂. 6H₂O 11.92, glucose 11.8, yeast extract 5.89, casamino acids 0.12, trace elements 2.35 ml (Hopwood et al., 1985), agar 25.9, after autoclaving the following solutions were added: 0.5% KH₂PO₄ 11.8 ml, 5 M CaCl₂ 4.71 ml, 1 N NaOH 8.25 ml) at 37° C. for several generations. Disruption mutants were selected based on the phenotype changing from apramycin and kanamycin resistant to apramycin sensitive and kanamycin resistant. Replacement of the chromosomal copy of the target gene with the dismpted plasmid-born copy was confirmed by Southern blot hybridization.

Mitomycin C Analysis

MC production was evaluated using 3-day cultures in Nishikohri media (Nishikohri and Fukui, 1978). The culture broth was extracted twice with equal volumes of ethyl acetate. After removing the chemical solvent by vacuum, the crude broth extract was dissolved in 50% methanol and 50% 50 mM pH 7.2 Tris buffer and monitored by HPLC (C₁₈ reverse phase column) at 363 nm. A continuous methanol gradient from 20% to 60% in methanol/50 mM pH 7.2 Tris buffer system over 24 minutes was employed to resolve MC from other crude extract components. A 90% CHCl₃/10% MeOH solvent system was used to resolve and detect MC on TLC plates.

Results

Identification of the Mitomycin Biosynthetic Gene Cluster

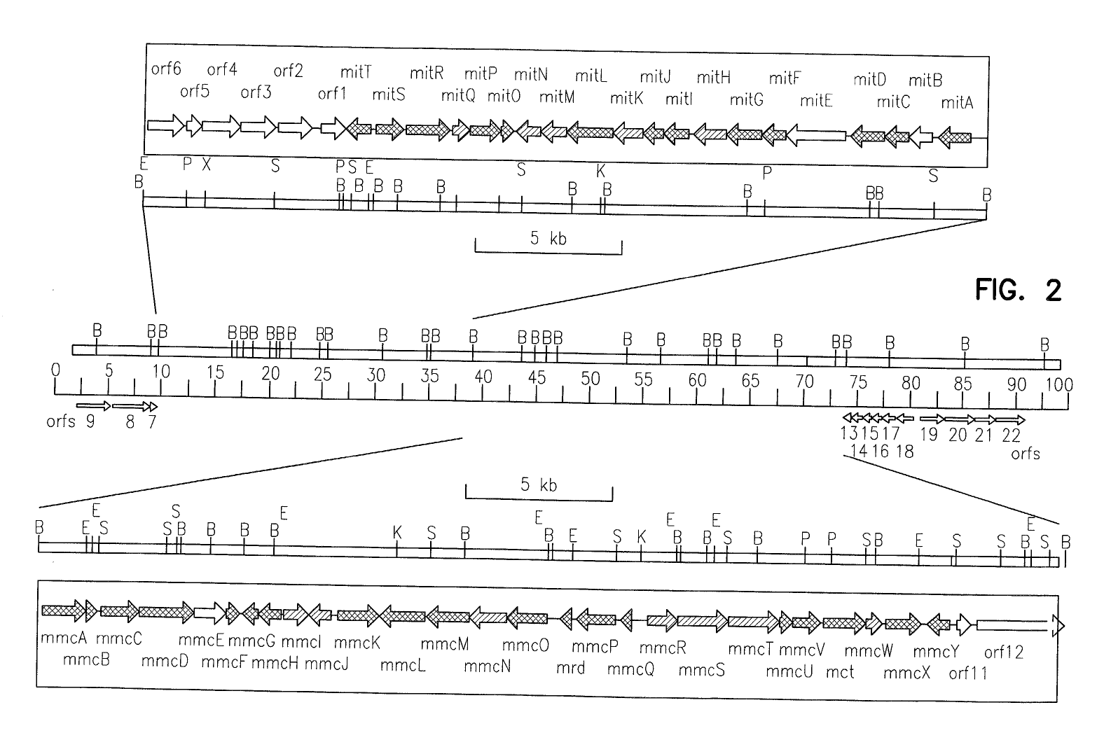

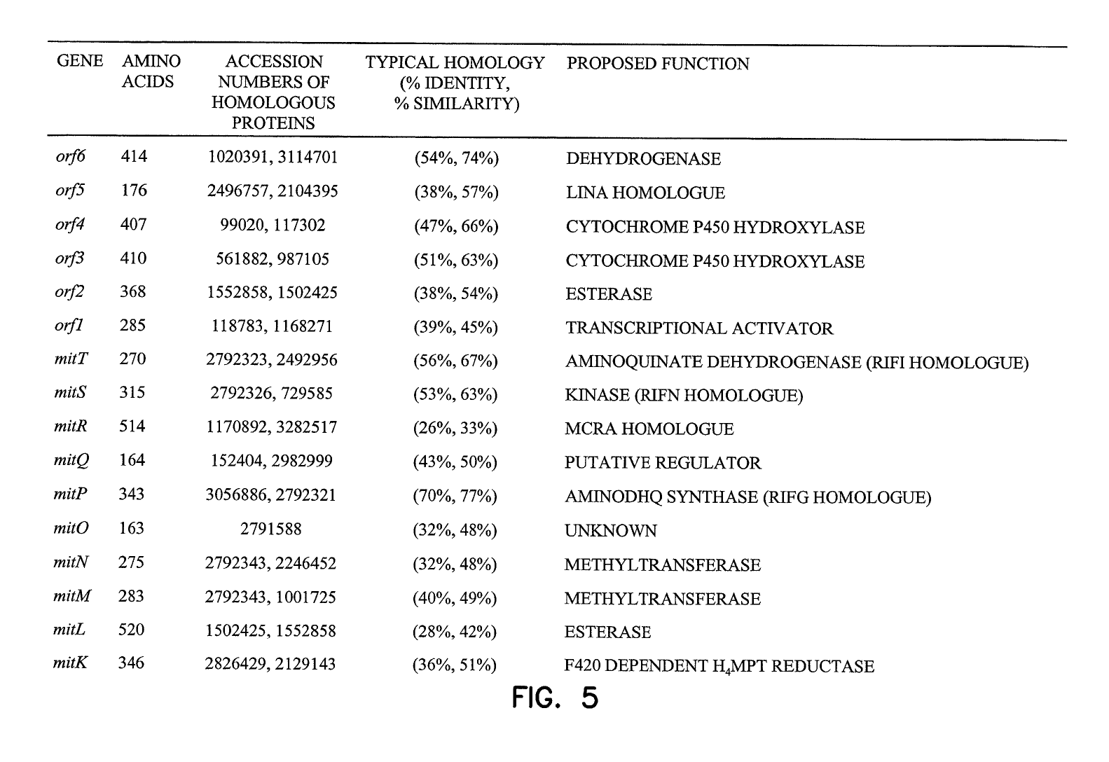

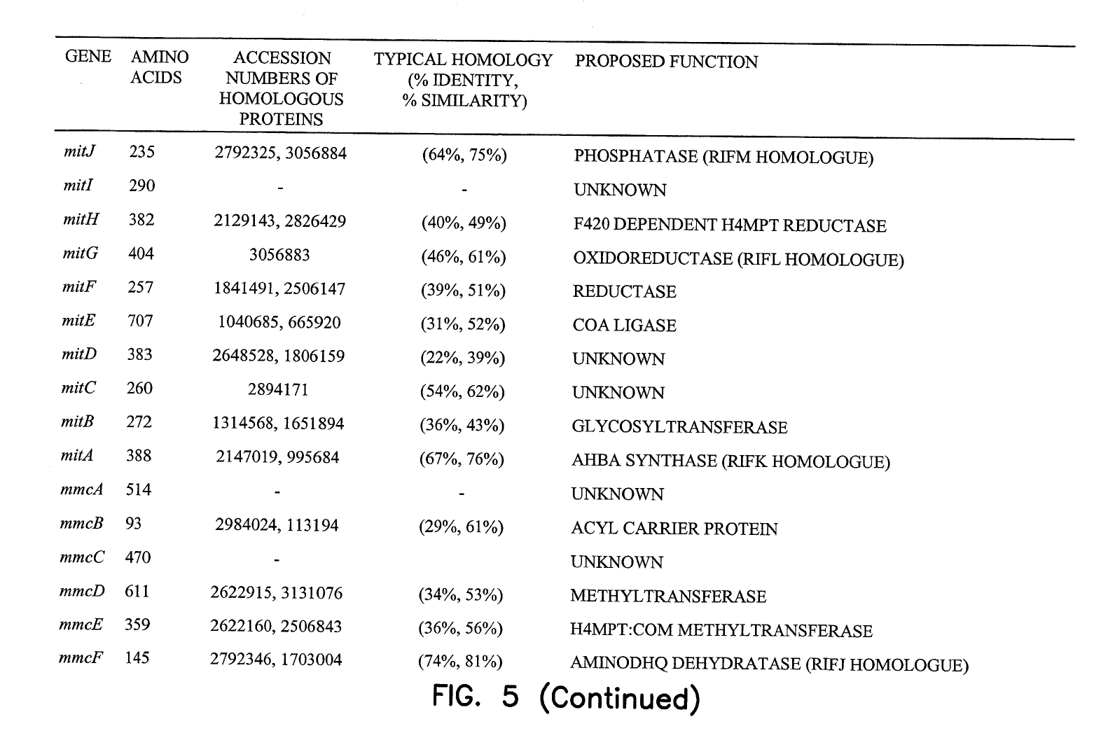

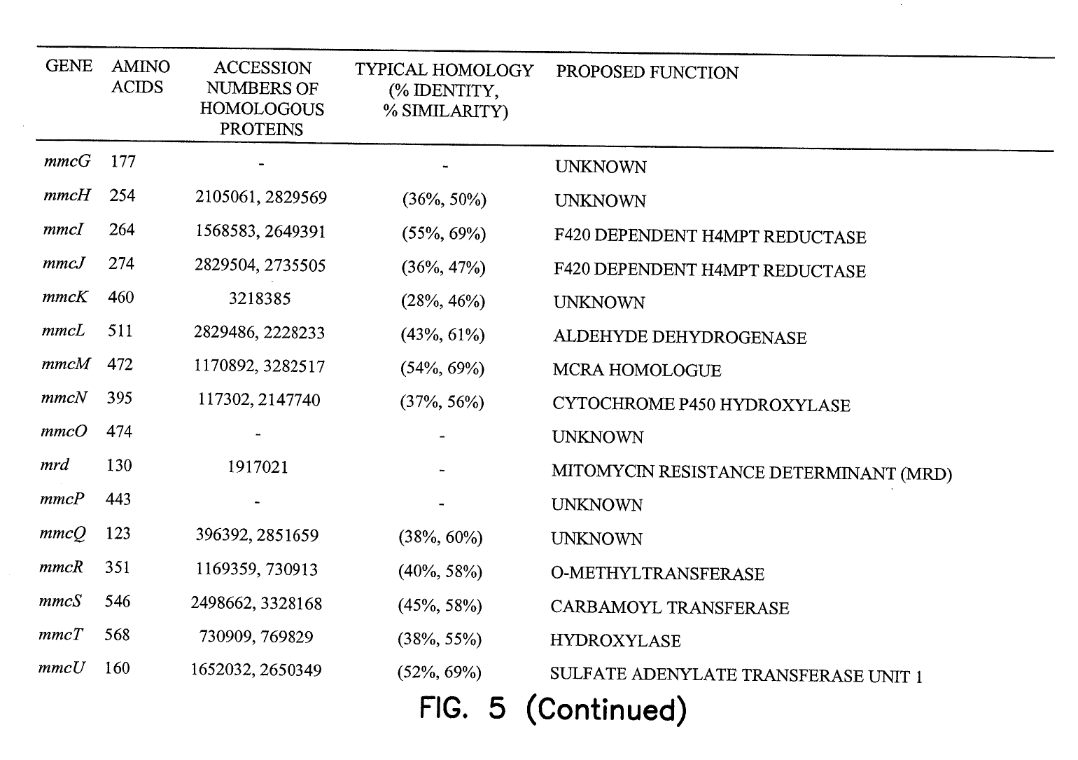

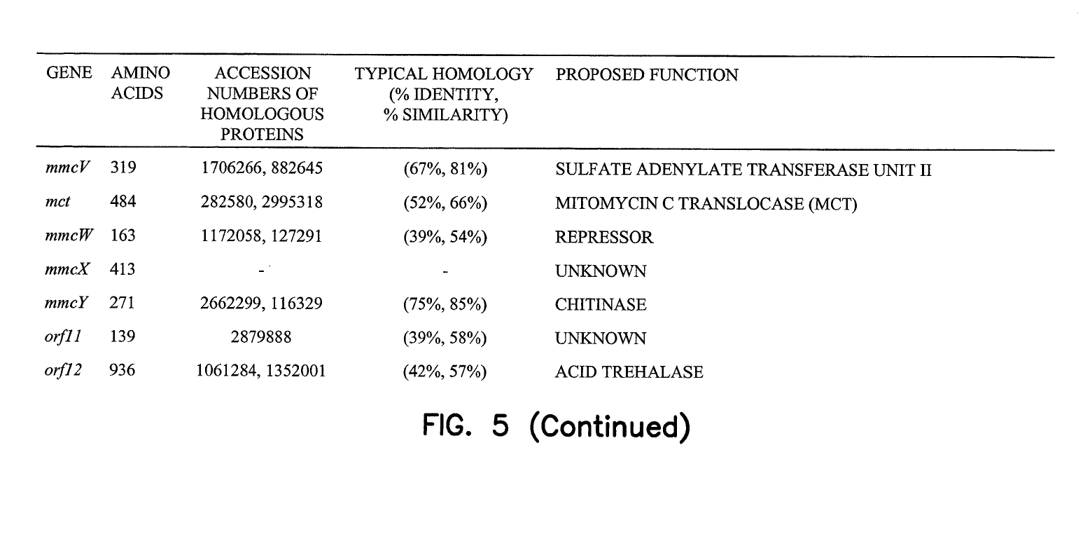

The mitomycin cluster was identified by linkage of a cosmid clone containing mrd and a gene (mitA) that hybridized with the rifK gene encoding the rifamycin AHBA synthase (Kim et al., 1998) from Amycolatopsis mediterranei. mitA was subsequently shown to be essential for mitomycin biosynthesis since genetic disruption of the chromosomal copy blocked MC production, and could be complemented with exogenous AHBA (Example 2). Linkage of mitA with one of the mitomycin resistance genes (mrd) implied that the corresponding biosynthetic genes were adjacent to mitA. Cosmid walking was used to obtain overlapping DNA fragments spanning more than 120 kb of the S. lavendulae chromosome adjacent to mitA. Subsequent nucleotide sequence analysis included 55 kb of contiguous DNA, revealing 47 genes involved in mitomycin assembly, regulation and resistance (FIGS. 2 and 5).

TABLE 1
MC production in wild-type S. lavendulae and
gene disruption mutants

| No. | gene disrupted | MC production |
|---|---|---|
| 0.0 | Wild-type control | ++ |
| 0.1 | additional copy | ++ |
| | of orf1 in | |
| | wild-type | |
| 1 | orf8 | ++ |
| 2 | orf4 | ++ |
| 3 | orf1 | ++ |
| 4 | mitR | + |
| 5 | mitM | − |
| 6 | mitI | − |
| 7 | mitH | − |
| 8 | mitE | − |
| 9 | mitB | − |
| 10 | mitA | − |
| 11 | mmcA | − |
| 12 | mmcB | − |
| 13 | mmcM | ++ |
| 14 | mmcP | − |
| 15 | mmcR | − |
| 16 | mmcT | − |
| 17 | mmcW | ++++ |
| 18 | mmcX | ++++ |
| 19 | orf11 | ++ |
| 20 | orf12 | ++ |
| 21 | orf16 | ++ |
| 22 | orf19 | ++ |

mitT Defines the Left-band Boundary of the Mitomycin Cluster

Nucleotide sequence analysis extended 30 kb downstream of mitA and revealed a set of genes corresponding to a type I polyketide synthase (PKS, orf9, SEQ ID NO:18; orf8, SEQ ID NO:19) and thioesterase (TEII, orf7, SEQ ID NO:20). MC is not derived from the polyketide pathway, and thusan orf8 disruption mutant showed normal MC production as expected (Table 1). Approximately 20 kb downstream of mitA, two genes (mitT, SEQ ID NO:29 and mitS, SEQ ID NO:30) encoding a putative aminoquinate dehydrogenase and glucose kinase, respectively, were located Both are believed to be involved in AHBA biosynthesis since their equivalents are also present in the rifamnycin biosynthetic gene cluster (rif cluster) (August et al., 1998). However, whether the six genes between orf7 and mitT are involved in MC biosynthesis remained unclear, since the two putative hydroxylases (orf3, SEQ ID NO:24 and orf4, SEQ ID NO:22) and the candidate activator gene (orf1, SEQ ID NO:26) could play a role in MC production. Both orf3 and orf4 are predicted to encode cytochrome P450 monooxygenases with Orf4 most similar to OleP and RapN (50% identity, 63% similarity) for oleandomycin and rapamycin biosynthesis, respectively (Rodriguez et al., 1995; Schwecke et al., 1995). Orf3 shows a high degree of similarity to cytochrome P450 105C1 (49% identity, 64% similarity) in Streptomyces sp. and cytochrome P450-SU2 in Streptomyces griseolus (Horii et al., 1990; Omer et al., 1990).

Database analysis revealed that Orf1 belonged to the ActII-ORF4, RedD, DnrI and CcaR family of Strepiomyces antibiotic pathway specific activators regulating the production of actinorhodin, undecylprodigiosin, daunorubicin, and cephamycin, respectively (Fernandez-Moreno et al., 1991; Perez-Laraine et al., 1997; Takano et al., 1992; Tang et al., 1996; Wietzorrek and Bibb; 1997). A common feature of this group of activators is that disruption of the corresponding gene abolishes the production of the corresponding antibiotic while overexpression results in a several-fold increase in metabolite production. However, when orf1 was disrupted, the mutant strain showed normal MC production (Table 1). Moreover, the wild-type MC producer containing an additional copy of orf1 in pKC1139 also had a normal MC production profile (Table 1). Interestingly, orf4, one of the cytochrome P450 monooxygenase encoding genes adjacent to orf1 also showed normal MC production when disrupted (Table 1). Thus, mitT appears to map to the left-hand end of the mitomycin cluster, while orf1 to orf9 presumably specify biosynthesis of a polyketide product.

mmcY Defines the Right-hand Boundary of the Mitomycin Cluster

Nucleotide sequence analysis of the mitomycin biosynthetic gene cluster extended 30 kb upstream of mitA and several orfs corresponding to genes involved in sugar metabolism were identified. They included an acid trehalase (orf12, SEQ ID NO:28), one ABC type transporter (orf16, SEQ ID NO:79), and four adjacent α-amnylases (orf19, SEQ ID NO:82; orf20, SEQ ID NO:83; orf21, SEQ ID NO:84; orf22, SEQ ID NO:85) for starch degradation spanning more than 18 kb (FIG. 2). Disruption of four genes (orf11, SEQ ID NO:27; orf12, SEQ ID NO:28; orf16, SEQ ID NO:79; orf19, SEQ ID NO:82) within this region resulted in mutants with wild-type level MC production profiles, indicating that they fall outside of the mitomycin cluster (Table 1). At the beginning of this group of sugar metabolism genes, a gene (mmcY, SEQ ID NO:75) encoding a presumed chitinase is proposed to be the upstream terminus of the mitomycin cluster. This is evident because mitomycin requires D-glucosamine as a biosynthetic precursor, and MmcY shows 75% identity (85% similarity) with the chitinase C gene (chiC) product from S. griseus that generates N-acetylglucosamine from chitin (Ohno et al., 1996). In addition, mutants with disrupted orf11 and orf12 genes had no effect on MC production, while disruption of mmcW(SEQ ID NO:71) and mmcX(SEQ ID NO:72) both affected MC production significantly (Table 1).

Mitomycin Resistance Genes

Antibiotic biosynthetic gene clusters typically include one or more genes for cellular self-protection (Seno and Baltz, 1989). Previous work has identified two mitomycin C resistance genes (mcr and mrd) with mrd linked to mitA (August et al., 1994; Sheldon et al., 1997; Example 2). Subsequent analysis showed that MRD is a resistance protein that binds mitomycin C with 1:1 stoichiometry (Sheldon et al., 1997). However, this resistance mechanism would be extremely inefficient unless the bound drug is transported out of the cell. Indeed, 5 kb upstream of mrd, the mct gene (SEQ ID NO:16, putative mitomycin translocase) encoding a presumed antibiotic transporter was found and shown to be a third resistance component (Example 3). mct encodes 484 amino-acid protein with 14 predicted transmembrane domains. Disruption of mct resulted in a mutant S. lavendulae strain substantially more sensitive to MC, while coexpression of mct with mrd in E. coli dramatically increased MC resistance levels compared to individual expression of the genes (Example 3). In contrast, the high-level MC resistance gene (mcrA) that encodes an MC oxidase (MCRA) capable of reoxidizing activated MC (Johnson et al., 1997) is not linked with this cluster (August et al., 1990; Example 2). Interestingly, database searches identified two McrA homologues (MitR, MmcM) within the MC cluster, both of which encode putative flavoproteins conserved in the FMN/FAD binding motif. MitR displayed weak similarity with McrA (26% identity, 33% similarity), while MmcM showed end-to-end (54% identity, 69% similarity) alignment with the protein. mitR (SEQ ID NO:31) and mmcM(SEQ ID NO:61) were genetically disrupted giving substantially decreased MC production in the mitR mutant strain, in contrast to the mmcM mutant which displayed wild type MC production levels (Table 1).

Regulatory Genes

Two genes, mitQ (SEQ ID NO:32) and mmcW(SEQ ID NO:71), were identified in the mitomycin cluster and are presumed to be pathway-specific regulators. MitQ belongs to the OmpR-PhoB subfamily of DNA binding regulators in the two-component regulatory system, with the greatest similarity to members of the phosphate assimilation pathway (PhoR-PhoB) (Makino et al., 1986), ferric enterobactin response pair (PfeR-PfeS) (Dean et al., 1996), and one histidine protein linase—response regulator system (HpkA-DrrA) from Thermotoga maritima (Lee and Stock, 1996). In contrast to the MitQ group of regulators that typically serve as transcriptional activators (Mizuno and Tanaka, 1997), MmcW showed high sequence similarity with the MarR groups of repressors. The most significant similarity corresponds to EmrR, the negative regulator of the E. coli multidrug resistance pump EmrAB (Lomovskaya et al., 1995), and Pacs, a repressor for pectinase, cellulase, and blue pigment production in Erwinia chrysanthemi (Praillet et al., 1996). Significantly, the mmcW disruption mutant displayed a several-fold increase in MC production (Table 1).

AHBA Biosynthetic Genes

Precursor incorporation studies previously demonstrated that AHBA is an intermediate for both the ansamycin and mitomycin natural products (Becker et al., 1983; Example 2). Combining the biochemical, enzymatic and molecular genetic results on the biosynthesis of the ansamycin antibiotic rifamycin, Floss has proposed that AHBA is derived from the ammoniated shikimate pathway via phosphenolpyruvate (PEP) and erythose 4-phosphate (E4P) by the early incorporation of nitrogen (Kim et al., 1996). In the shikimate pathway, PEP and E4P is first converted to 3deoxy-D-arabino-heptulosonic acid-7-phosphate (DBAHP) then stepwise transformed to 3dehydroquinate (DHQ), 3-dehydroshikimate (DHS) and shikimate, catalyzed by DAHP synthase, DHQ synthase, DHQ dehydratase, and shikimate dehydrogenase, respectively (Dewick, 1998). Quinate can also enter the pathway by the action of quinate dehydrogenase to generate DHQ.

Evidence to support this new variant of the shikimate pathway includes the following experimental observations. First, all proposed ammoniated shikimate pathway compounds including PEP, E4P, 3,4-dideoxy-4-amino-D-arabino-heptulosonic acid 7-phosphate (aminoDAHP), 5-deoxy-5-amino-3-dehydroquinic acid (aminoDHQ), and 5-deoxy-5-amino-3-dehydroshikimic acid (aminoDHS) can be readily converted into AHBA by cell-free extracts from the ansamycin producers, while none of the early shikimate pathway intermediates, DAHP, DHQ, DHS, quinic acid, shikimic acid can be incorporated into AHBA under the same conditions (Homemann, 1981; Kim et al., 1996). Second, the rifamycin biosynthetic gene cluster (rif cluster) has been sequenced, and all of the genes encoding early shikinmate pathway enzymes are found within the cluster (August et al., 1998). Finally, the ability of the rifamycin AHBA synthase (RifK) to catalyze dehydration of aminoDHS to AHBA has been previously demonstrated (Kim et al., 1998). As described in Example 2, the AHBA synthase gene (mitA) in S. lavendulae is required for AHBA biosynthesis.

A group of AHBA biosynthetic genes similar to those described for nf have been identified in the mitomycin cluster. In addition to AHBA synthase, six gene products in the cluster showed high sequence similarity (over 43% identity) with their rifamycin AMBA biosynthetic gene homologs. These gene products include aminoDHQ synthase (MitP, RifG equivalent), aminoquinate dehydrogenase (MitT, RifI equivalent), oxidoreductase (MitG, RifL, equivalent), phosphatase MitJ, RifM equivalent), kinase (MitS, RifN equivalent), and aminoDHQ dehydratase (MmcF, RifJ equivalent). In addition to the significant sequence similarity to rifamycin counterparts, all three putative mitomycin shikimate pathway enzymes displayed significant alignment with microbial primary shikimate metabolic enzymes including MitT with the quinate dehydrogenase (AroE) from Methanococcus jannaschii (28% identity, 46% similarity) (Bult et al., 1996), MitP with the DHQ synthase (AroB) from Mycobacterium tuberculosis (46% identity, 61% similarity) (Cole et al., 1998), and MmcF with the DHQ dehydratase from S. coelicolor (50% identity, 62% similarity) (White et al., 1990). Despite extensive sequencing of 15 kb on either side of the mapped right- and left-hand ends of the mitomycin cluster, an aminoDAHP synthase gene corresponding to RifH (the proposed first enzyme in the de novo biosynthesis from PEP and E4P in the rif cluster), was not found (FIG. 2). Interestingly, a rifH homologue has been cloned from S. lavendulae genomic DNA through Southern hybridization and shown to be unlinked to the mitomycin cluster.

The existence of non-shikimate pathway-related phosphataselkinase pair in the mitomycin cluster (MitJ/MitS) and the rif cluster (RifM/RifN) further support the finding that these two genes are required for AHBA biosynthesis (Floss, 1997). In addition to the strong homology to RifM, MitJ also showed 56% identity (69% similarity) with ORF8 from the ansamycin antibiotic ansamitocin producer Actinosynnema pretiosum auranticum. Other polypeptides with considerable sequence similarity belong to the CBBY family of phosphoglycolate phosphatases in glycolate oxidation (Schaferjohann et al., 1993). MitS, most similar to RifN (53% identity, 63% similarity), also showed significant similarity with the glucose kinase (involved in glucose repression) from S. coelicolor and Bacillus megaterium (Angell et al., 1992; Spath et al., 1997). mitG, the third non-shilimate pathway-related AHBA biosynthetic gene in this cluster is also worthy of note since it shows exclusive similarity (46% identity, 61% similarity) with oxidoreductase RifL, and its equivalent in Actinosynnema pretiosum auranticum.

Mitosane Fonnation Genes

Precursor incorporation studies established that the mitosane core is assembled form the condensation of AHBA and D-glucosamine. Although no specific gene products can be assigned for forming the three bonds bridging AHBA and D-glucosamine, two genes downstream of mitA (SEQ ID NO:97), mitB (SEQ ID NO:99), and mitE (SEQ ID NO:44) likely encode enzymes that mediate one of these reactions. MitB shows local sequence similarity with a group of glycosyltransferases involved in glycopeptide antibiotic and polysaccharide biosynthesis, the typical function of which is to attach an activated sugar residue to a core compound (Yamazaki et al., 1996,Example 2). Meanwhile, MitE showed weak similarity (22% identity and 45% similarity) to the two cloned 4-hydroxybenzoate-CoA ligases from Rhodopseudomonas palustris in the anaerobic degradation of aromatic compounds (Gibson et al., 1994). It also showed similarity to a group of long chain fatty acid CoA ligases, as well as to the O-succinylbenzoic acid CoA synthetase in Vitamin K2 biosynthesis (Kwon et al., 1996). mitB and mitE disruption mutants both had a MC deficient phenotype (Table 1).

The condensation of AHBA with D-glucosamine may be initiated in two different ways. This includes either initial formation of a C₈ₐ-C₉ bond by an acylation or alkylation reaction, or formation of a Schiff base between the MABA nitrogen and D-glucosamine C1 aldehyde, followed by the ring closure at C₈ₐ-C₉. mitR (SEQ ID NO:31), one of the two McrA homologues may be involved in one of the ring closure reactions. Interesting, MitR showed high sequence homology with the plant berberine bridge enzyme (BBE) (30% identity, 37% similarity) in benzophenanthridine alkaloid formation, where it catalyzes an unusual C-C bond formation of the berberine bridgehead carbon of (S)-scoulerine from the N-methyl carbon of (S)-reticuline (Dittrich and Kutchan, 1991). Using a mechanism similar to BBE, it is possible that MitB is involved in C₈ₐ-C₉ bond formation. The decreased MC production in the mitR disruption mutant may be due to the existence of isoenzymes (e.g., MmcM) that could catalyze the reaction in the absence of a functional MitR

Side Group Modification Genes

Complete assembly of MC requires functionalization of several sites on the core mitosane ring system. First, complete reduction of the carbonyl group at C-6 must occur. Second, hydroxylation at C-5 and C9a must proceed followed by methylation at C-9a. Third, amination at C-7 must occur presumably through initial hydroxylation followed by transamination. Fourth, oxidation of the hydroxyl groups at C-5 and C-8 to form the benzoquinone are required. Fifth, intramolecular amination of C-1 by N-1a to form the aziridine ring must be completed and finally, carbamoylation at C-10 completes assembly of the molecule. Several enzymes found in this cluster likely catalyze these modifications and are discussed below.

Methylation

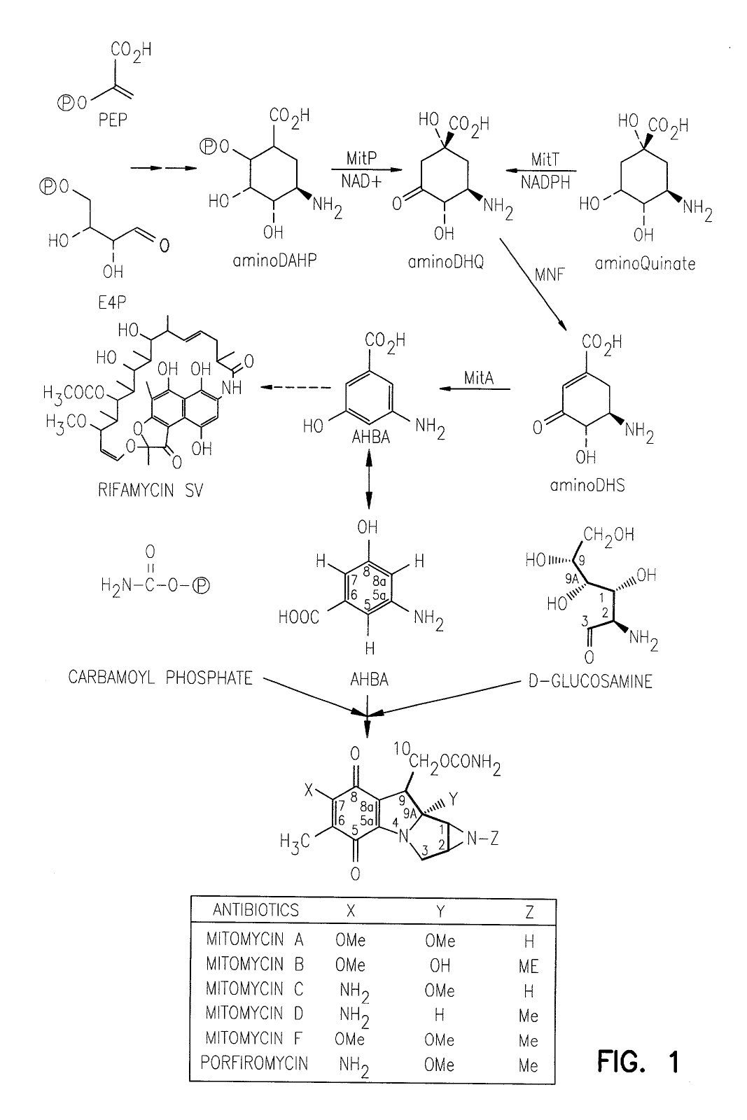

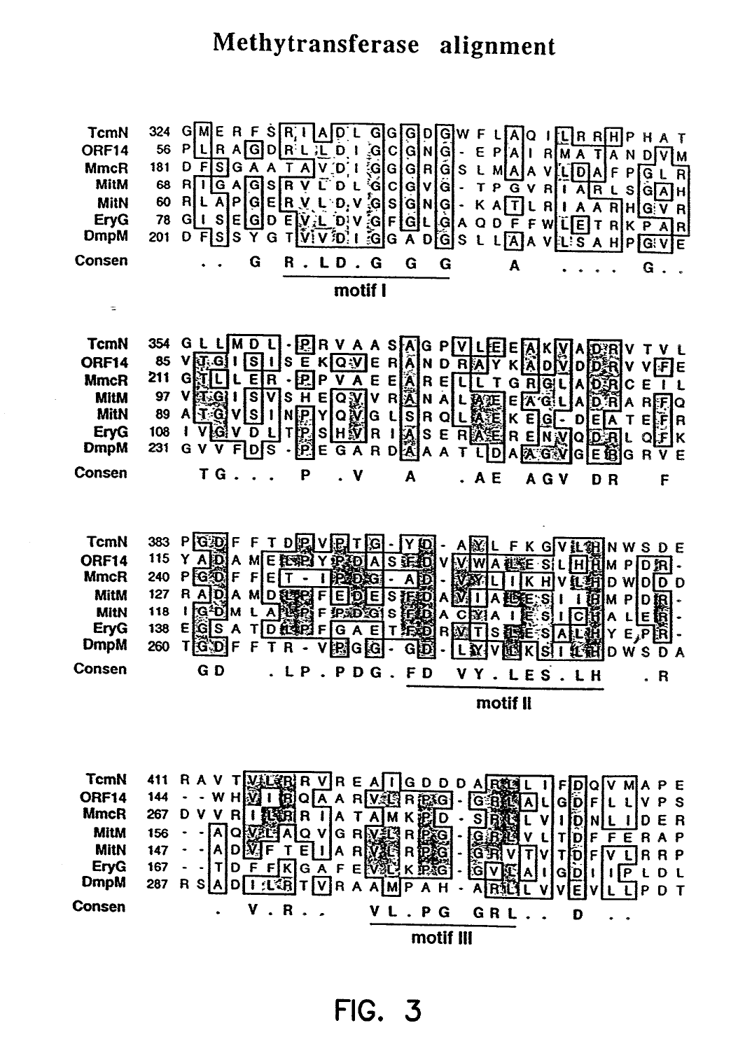

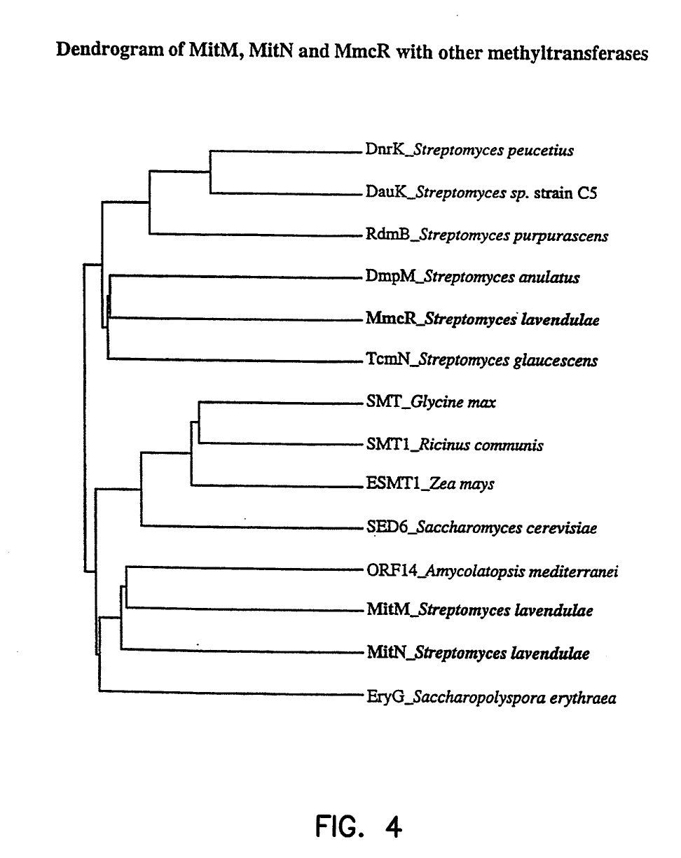

In contrast to MC which has an O-methyl group at C-9a, mitomycin A and mitomycin B also contain a C-7 O-methyl group, while mitomycin B, mitomycin D and porfiromycin have an N-methyl on the aziridine ring (FIG. 1). Radio-labeled precursor incorporation studies showed that all of the O- and N-methyl (but not the C-methyl) groups in the mitomycin molecules are derived from L-methionine (Bezanson and Vining, 1971). Typically, the methyl donor for most C1 reactions is S-adenosyl-L-methionine (SAM), which can be formed through activation of L-methionine by ATP. Three SAM dependent methyltransferase genes were identified in this cluster (encoding MitM, MitN, and MmcR), all of which have three conserved S-adenosylmethionine or S-adenosylhomocysteine binding motifs (Kagan and Clarke, 1994) (FIG. 3). Interestingly, database searches of MitM and MitN (likely responsible for the MC C-9a side chain methylation) revealed a group of plant δ-(24)-sterol C-methyltransferases that have a closer phylogenetic relationship with the rifarnycin O-methyltransferase (ORF14) and erythromycin O-methyltransferase (EryG) (5, 86) (FIG. 4). In contrast, protein database searches revealed that MmcR is most related to other Streptomyces antibiotic biosynthetic O-methyltransferases with greatest similarity to O-demethylpuromycin O-methyltransferase (44% identity, 60% similarity) from S. anulatus and carminomycin 4-O-methyltransferase from S. peucetius (Lacalle et al., 1991; Madduri et al., 1963). MmcR may be involved in the O-methylation of the phenol ring of MC before oxidation to the quinone. Both mmcR (SEQ ID NO:67) and mitM (SEQ ID NO:36) were shown to be essential for MC biosynthesis since disruption of each one completely abolished MC production (Table 1).

A SAM-independent methyltransferase, MmcD, was also identified in the mitomycin cluster. MmcD revealed strong sequence homology with the magnesium-protoporphyrin IX monomethyl ester oxidative cyclase (34% identity, 53% similarity) from Methanobacterium thermoautotrophicum (Accession Number 2622915), as well as the phosphonoacetaldehyde methyltransferase from Streptomyces wedmorensis (Hidaka et al., 1995), the P-methyltransferase from Streptomyces hygroscopicus (Hidaka et al., 1995) and the fortimicin KL methyltransferase from Micromonospora olivasterospora (Kuzuyama et al., 1995). Instead of SAM, this group of methyltransferases uses methylcobalamine or a structurally related protoporphyrin as the direct methyl donor. While the greatest number of matches were made to protoporphyrin methyltransferases, it is expected that this enzyme has another function in the mitomycin C biosynthetic pathway as all the O- and N-methyl groups of MC have been shown to be derived from SAM-dependent methyltransferases.

C-6 Carbonyl Reduction

The C-6 methyl group was previously shown to be derived from the reduction of the carboxylic acid of AHBA, since [carboxy-¹³C] AHBA can be efficiently, and specifically incorporated into the C-6 methyl group of porfiromycin (Anderson et al., 1980). In the mitomycin cluster, four F420-dependent tetrahydromethanopterin (H₄MPT) reductase genes (encoding MitH, MitK, MmcI, MmcJ) and one H₄MPT:CoM methyltransferase gene (encoding MmcE) are candidates for the C-6 carbonyl reduction. In the methanogenesis pathway of Methanobacterium thermoautotrophicum, two cofactor F420-dependent H₄MPT reductases, and one cofactor CoM dependent methyltransferase are required in the seven step reduction from CO₂ to CH₄. Steps 4 to 6 from CH-H₄MPT to CH₂-H₄MPT, and CH₃-H₄MPT to CH₃-CoM are catalyzed by N⁵, N¹⁰-methylene-H₄MPT dehydrogenase, N⁵, N¹⁰-methylene-H₄MPT reductase, and N⁵-methyl-H₄MPT:CoM methyltransferase, respectively (Depperuneier et al., 1996; Thauer et al., 1993). All four enzymes (MitH, MitK, MmcI, MmcJ) in this cluster showed local sequence similarity with the cloned F420 dependent H₄MPT reductase (42% identity, 62% similarity in several 50 amino-acid fragments) (Nolling et al., 1995; Vaupel and Thauer 1995). One of these genes, mitH (SEQ ID NO:41) was disrupted, and the mutant strain displayed a MC deficient phenotype (Table 1). MmcE is notable since the deduced protein sequence contains two domains showing significant alignment (33% identity, 56% similarity) to the N-terminus of H₄MPT:CoM methyltransferase from Methanobacterium thermoautotrophicum (Stupperich et al., 1993), while the remaining C-terminus is related to fatty acid biosynthetic acyl carrier proteins (ACP) (Morbidoni et al., 1996; Platt et al., 1990). The potential function of this ACP-like domain in MC biosynthesis remains unknown, as does the role of a distinct gene (mmcB, SEQ ID NO:50) encoding a putative ACP identified just upstream of mmcE (SEQ ID NO:53). Significantly, the disruption of mmcB resulted in total abrogation of MC production (Table 1).

Hydroxylation

The two putative hydroxylases (encoded by mmcN, SEQ ID NO:62; and mmcT, SEQ ID NO:69) identified in the mitomycin cluster are candidates for catalyzing hydroxylation at the C-5, C-7, and C-9a positions on the mitosane system. MmcN belongs to the cytochrome P450 family of monooxygenases, with greatest homology (37% identity, 56% similarity) to the two herbicide-inducible cytochrome P450s (P450-SU1 and P450-SU2) from S. griseolus, as well as to RapJ and RapN in the rapamycin biosynthetic gene cluster from S. hygroscopicus (Omer et al., 1990; Schwecke et al., 1995). MmcT showed highest similarity to the tetracenomycin C hydroxylase (TcmG) in Streptomyces glaucescens (38% identity, 55% similarity), with lower but significant sequence similarity to a group of phenol or hydroxybenzoate hydroxylases (Decker et al., 1993). Genetic disruption of mmcT completely blocked MC biosynthesis (Table 1)

Carbamoylation

The carbamoyl group of MC is derived intact from L-citrulline or L-arginine with carbamoyl phosphate as the incorporated precursor (Homemann, 1981). In eubacteria, carbamoyl phosphate can be generated from L-glutamine, HCO₃⁻, and ATP by the enzyme carbamoyl phosphate synthetase, which is indispensable for pyrimidine biosynthesis. One candidate carbamoyl transferase gene (mmcS, SEQ ID NO:68) was identified directly upstream of mmcT. MmcS belongs to the NodU/CmcH family of O-carbamoylation enzymes, with the greatest similarity (35% identity, 44% similarity) to NolO from Rhizobium sp. (Jabbouri et al., 1998). Other members with significant alignment in this family include NolO from Bradyrhizobium japonicum (Luka et al., 1993) and NodU from Rhizobium sp. for 6-O-carbamoylation of Nod-factors (Jabbouri et al., 1995) and CmcH from Nocardia lactamdurans and S. clavuligerus for 3′-hydroxyrnethylcephem O-carbamoylation in cephamycin biosynthesis (Coque et al., 1995).

Discussion

Bridging Primary and Secondary Metabolism

The shikimate pathway is an essential metabolic route in microorganisms and plants for aromatic amino acid biosynthesis. Genes encoding the early shikimate pathway enzymes from various organisms have been well studied and are often dispersed along the chromosome as revealed by genome sequencing projects (Blattner et al., 1997; Bult et al., 1996; Cole et al., 1998). The finding that the ansamycin and mitomycin natural products are derived in part from an ammoniated shikimate pathway whose genes are clustered on the bacterial chromosome is a significant difference to the primary metabolic network, and may suggest an important evolutionary bridge leading to secondary metabolism. The lack of incorporation of early shikimate pathway intermediates into mitomycin and ansamycin metabolites indicated the existence and ultimate substrate specificity of the alternate ammoniated shikimate pathway enzymes. However, the conversion of aminoDAHP and aminoshikimic acid by the corresponding primary shikimate pathway enzymes to aminoDHQ and aminoDHS, respectively (Kim et al., 1996), suggested that the substrates specificity in primary metabolic shikimate pathway is mainly determined by the initial reaction step. This notion is further supported by the disruption results for rifG and rifI mutants showing only slightly affected rifamycin production (Floss, 1997).

In addition to the absence of an aminoDAHP synthase gene, the organization of the AMBA biosynthetic genes in the MC cluster is quite different compared to the rif cluster. In rif (with the exception rifJ), all AMBA biosynthetic genes are found within a defined subcluster that are organized into a single apparent operon. In contrast, almost all of the mit/mmc encoded AHBA genes are scattered within the 55 kb MC cluster. Thus, as opposed to the multifunctional polyketide gene clusters whose linearity of architecture reflects a precise pattern of biosynthesis, the MC cluster is biochemically less transparent based on a similar primary analysis. In addition, the MC cluster provides a good model for analyzing genetic evolution both vertically, from the primary metabolic shikimate pathway to the secondary shikimate pathway related route, and horizontally by comparing different groups of secondary metabolic biosynthetic clusters.

The MC Biosynthetic Network

In a typical liquid culture of S. lavendulae, MC production initiates 24 hours after inoculating the seed culture, reaches maximum production in two days, and maintains drug synthesis during stationary phase for another two days. Compared to high level MC resistance of the wild-type S. lavendulae (>150 μg(ml), MC production is relatively low (<5 μg/ml MC). The significant gap between the self-resistance and production levels makes it possible to improve drug production through genetic engineering. As described herein, disruption of the candidate repressor gene (mmcW) and downstream mmcX(encoding a putative membrane protein) in the mitomycin cluster resulted in a several-fold increase in MC production. The existence of a repressor gene(s) is not uncommon in Streptomyces antibiotic biosynthetic gene clusters. Previous examples include, mmyR from the methylenomycin cluster (Chater and Briton, 1985), actII-orfI in the actinorhodin cluster (Caballero et al., 1991),jadR (Anderson et al., 1980) in jadomycin biosynthesis (Yang et al., 1995), and dnrO in the daunorubicin cluster (Otten, 1995). Disruption of jadR and mmyR also resulted in increased levels of the corresponding antibiotic (Chater and Briton, 1985; Yang et al., 1995).

In order to avoid auto-toxicity, drug-producing microorganisms must evolve self-protection systems. Currently, three types of self-protection mechanisms have been identified in S. lavendulae for mitomycin resistance including, MC binding (MRD), efflux (MCT), and reversing MC reductive activation (MCRA). In principle, resistance genes must be expressed before drug formation. In this respect, it is interesting to note the linkage of the mitomycin resistance genes with the regulatory genes. Expression of the high-level resistance gene mcrA has been demonstrated to be regulated by the downstream gene mcrB which is presumably cotranscribed with mcrA (August et al., 1994). Though the function of the McrA homolog MitR in the mitomycin cluster remains unknown, mitR is also followed by a cotranscribed regulatory gene (mitQ). Meanwhile, the putative mitomycin translocase gene, mct is followed by the repressor gene, mmcW. Genetic linkage of membrane transporter/resistance and repressor genes have been described in a number of cases, including tetA/tetR in tetracycline resistance (Guilfoile and Hutchinson, 1992), tcmA/tcmR in tetracenomycin C resistance (Guilfoile and Hutchinson, 1992), actII-orf2/actII-orf1 in actinorhodin resistance (Caballero et al., 1991), and the qacA/qacR pair for multidrug resistance in S. aureus (Grkovic et al., 1998).

Conclusion

Although MC was first isolated more than 40 years ago and has been used in anti-cancer chemotherapy since the 1960s, the mechanistic details and order of its biosynthesis has remained unclear. The results described herein are clearly consistent with precursor incorporation studies gathered in the 1970s, showing that MC is biosynthetically derived from D-glucosamine, L-methionine, carbamoyl phosphate, and AHBA, and also support the use of the variant de novo shikimate pathway leading to AHBA (Homemann, 1981; Kim et al., 1996). Many, if not all, of the genes responsible for the formation of the mitosane and aziridine rings are evidently located within the boundary of the 55 kb mitomycin cluster. These genes are of special interest since they may be useful as probes for identification of related natural product biosynthetic genes from other microorganisms and plants.

The cloned genes presented here are useful to study mitomycin biosynthesis and natural product assembly. The advantage of having this information has already been demonstrated through genetic disruption of the candidate repressor gene (mmcW) that provided a several-fold increase in MC production. In addition, expression and genetic disruption of selected genes should be useful for engineering the biosynthesis of clinically valuable mitomycin analogues, as well as more complex hybrid natural product systems. Finally, the MC resistance and regulatory genes identified in this cluster provide important insight into the mitomycin biosynthetic and regulatory network in the S. lavendulae.

### EXAMPLE 2

### Genetic Localization and Molecular Characterization of Two Genes Required for MC Biosynthesis

Materials and Methods

Strains and culture conditions. E. coli DH5α was grown in either Luria broth (LB) or tryptic soy broth (TSB) (Difco) as liquid medium or agar plates. E. coli DH5αF′, the host for harvesting single-stranded DNA, was grown at 37° C. on TBG (1.2% tryptone, 2.4% yeast extract, 0.4% glycerol, 17 mM KH₂PO₄, 55 mM K₂HPO₄, and 20 mM glucose). E. coli S17-1 (Mazodier et al., 1989) used for conjugation was grown in TSB with 10 μg/ml of streptomycin. S. lavendulae was grown in TSB or on R5T plates. For MC production, S. lavendulae was grown in Nishikohri media (g/L: glucose 15, soluble starch 5, NaCl 5, CaCO₃ 3, yeast extract 5) for 72 hours from a 1% v/v inoculum of frozen mycelia. Pulse feeding of AHBA to the disruption mutant, MV100, and the site-directed mutant, MV102, occurred with feedings of 2.5 mg of a 20 mg/mL solution of the sodium salt of AHBA at pH 7.1 in three pulses at 24, 43, and 57 hours of a culture that was harvested at 76 hours.

DNA preparation and amplification. Isolation and purification of DNA was performed using standard methods (Sambrook et al., 1989). S. lavendulae NRRL 2564 genomic DNA was isolated by using the modified Chater protocol (Hopwood et al., 1988). Plasmid DNA was isolated from E. coli by using the alkaline-sodium dodecyl sulfate method.

pDHS2002 was constructed as follows: The 1.1 kb thiostrepton resistance gene (tsr) fragment was removed from pDHS5000 with a SmaI-BamHI digestion, blunt-ended with the large fragment of DNA polymerase (Gibco BRL), and ligated to MscI restriction enzyme digested pDHS7601 to yield pDHS20001. MscI digestion of pDHS7601 resulted in the removal of 155 nucleotides at the C-terminus of the mitA gene, and ligation of the blunt-ended BamHII site of the tsr adjacent to the MscI site of pDHS7601 resulted in regeneration of the BamHI site in pDHS2001. The 4.9 kb EcoRI-HindIII fragment from pDHS2001 containing the tsr disrupted mitA gene was removed and ligated into EcoRI-HindIII digested pKC1139 to yield pDHS2002.

Primer-mediated site-directed mutagenesis (SDM) was employed to construct pDHS2015 containing a K191A mutation in mitA. Primer 1: 5′-GGCAAGGCATGCGAGGGTCGC-3′ (SEQ ID NO:46) and primer 2: 5′-TTCCAGAACGGCGCCCTGATGACCGCCGGC-3′ (SEQ ID NO:47) were used to amplify the 691 bp fragment of the 5′ end of mitA. The 3′ end of mitA was amplified with primer 3: 5′-GCCGGCGGTCATCAGGGCGCCGTTCTGGAA-3′ (SEQ ID NO:48) and primer 4:5′-TCAGAATTCGGATCCGAGGGCCGGAGT-3′ (SEQ ID NO:86) to generate a 1151 bp band (see amplification reaction conditions in Example 3). A second round of PCR was performed using the overlapping 691 and 1151 bp units as the initial templates with primer 1 and primer 4 to yield a 1.8 kb fragment. The final product containing mutagenized mitA was digested with EcoRI-Sph1, ligated to the 2.1 kb HindII-SphI fragment from pDHS7601 and the EcoRI-HindIII digested pKC1139 to yield pDSH2015. The site-directed mutation of MitA K191A in pDHS2015 was confirmed by sequencing with forward primer: 5′-ACCTACTGCCTCGATGCC-3′ (SEQ ID NO:87) and reverse primer: 5′-CTGATCCTTCAAGCG-3′ (SEQ ID NO:88).

The mitB disruption vector pDHS7702 was constructed as follows. pDHS7601 was digested with BstBI, blunt-ended, and ligated with the 1.4 kb neomycin-resistant gene fragment from pFD666 (Denis and Brzezinski et al., 1992) (ApaL1-HindIII digestion, blunt-ended). The 5.2 kb EcoRI-HindIII fragment from the resulting construct pDHS7701 was subeloned into pKC1139 to create pDHS7702.

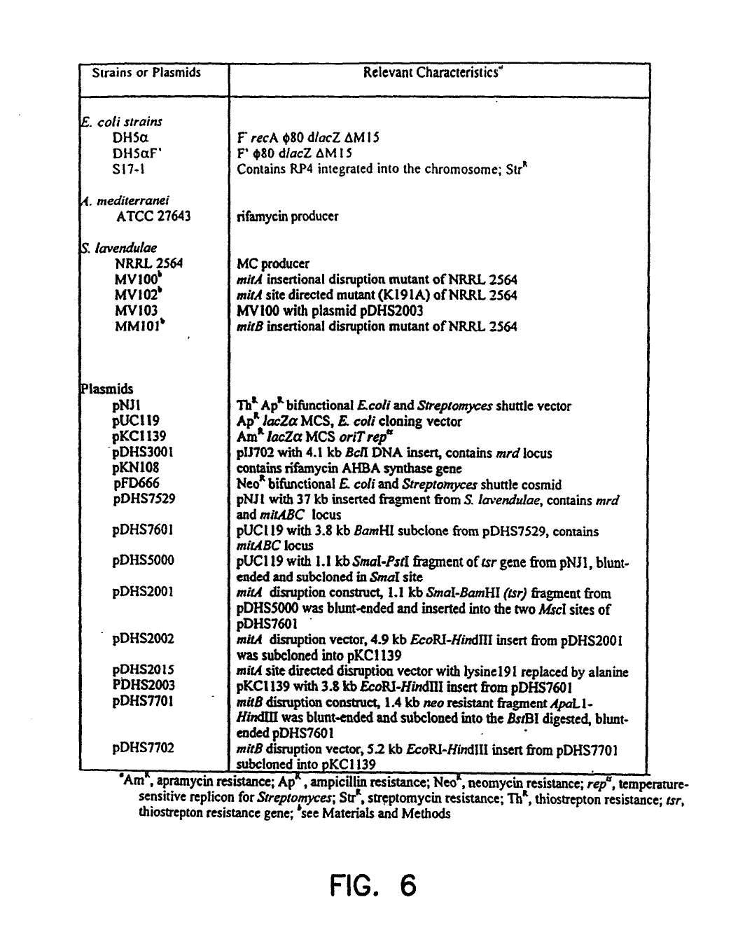

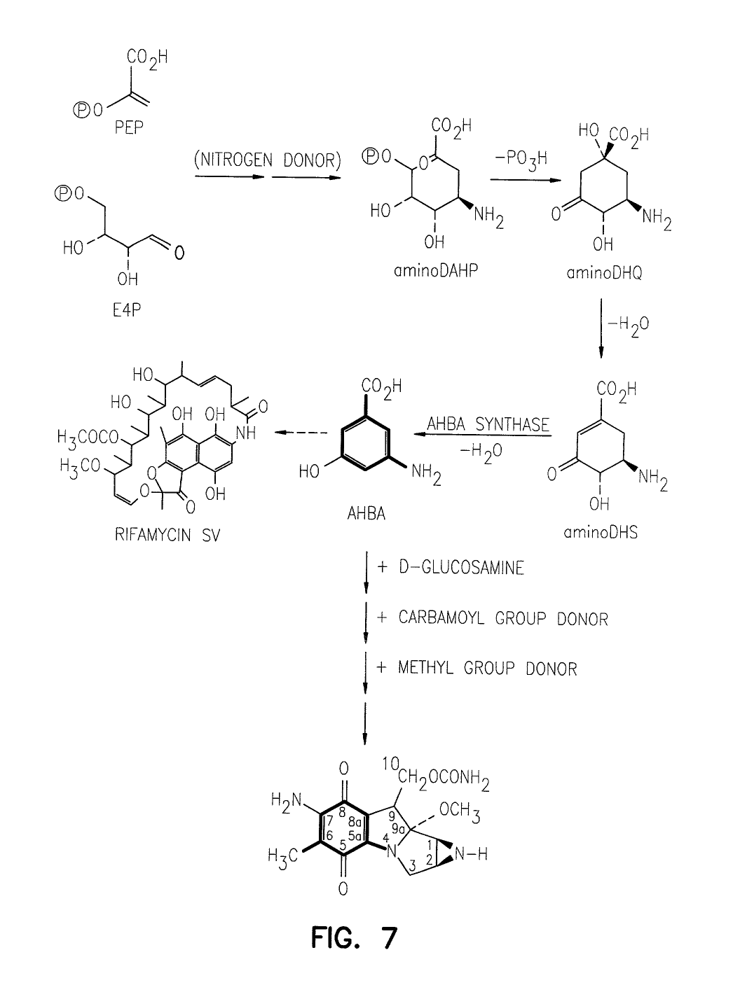

DNA library construction and screening. S. lavendulae NRRL 2564 genomic DNA was partially digested with Sau3AI, and a fraction containing 30-50 kb fragments was recovered by sucrose gradient centrifugation and ligated into the calf intestinal alkaline phosphatase (CIP) treated BglII site of the E. coli-Streptomyces shuttle vector pNJ1 (Tuan et al., 1990), then packaged with the Packagene Lambda DNA Packaging System (Promega). The cosmid library was constructed by transfecting E. coli DH5α, and colonies that appeared on the LB plates containing 100 μg/ml of ampicillin were transferred to a BioTrace NT nitrocellulose blotting membrane (Gelman Sciences, Ann Arbor, Mich.). Colony hybridization was performed as specified by the manufacturer. A PCR-amplified 0.7 kb DNA fragment from plasmid pKN108 (FIG. 6) was used to screen the library. The primers used for PCR were: 5′-GCGTCCGTGCTGCGCGCGCA-3′ (SEQ ID NO:89), and 5′-TGCGCGCGCAGCACGGACGC-3′ (SEQ ID NO:90). The cosmids from the positive colonies were confirmed by Southern blot hybridization, and a 1.7 kb AflIII-BamHI fragment from pDHS3001 containing the mitomycin resistance determinant (mrd) (Sheldon et al., 1997) was used as a probe to establish genetic linkage.

DNA sequencing and analysis. Deletion subclones from pDHS7601 were made with exonuclease III Erase-a-Base System (Promega). Sequencing was accomplished with the ABI PRISM™ Dye Terminator Cycle Sequencing Ready Reaction Kit (Applied Biosystems), and analyzed on an Applied Biosystems 377 DNA Sequencer at the University of Minnesota Advanced Genetic Analysis Center. For generating single-stranded DNA, deletion subclones in pUC119 were transformed into E. coli DH5αF′, and M13K07 Helper Phage was used (GIBCO BRL). Nucleotide sequence data were analyzed using Wisconsin Genetics Computer Group software (version 9.0) (Devereux et al., 1984), and GeneWorks software version 2.51 (Oxford Molecular Group). The GenBank accession number for mitABC is AF115779.

Conjugation from E. coli S17-1 to S. lavendule. The procedure of Bierman et al. (Bierman et al., 1992) was used with the following modification. A single colony of E. coli S17-1/pDHS2002 was used to inoculate 2 ml of TSB containing 100 μg/ml of apramycin and 10 μg/ml of streptomycin. Following overnight incubation at 37° C. a 1:100 inoculation was made into TSB broth with 100 μg/ml of apramycin and 10 μg/ml of streptomycin. This culture was grown for 3 hours at 37° C., and the cells were washed twice with TSB and resuspended in 2 ml of TSB to provide the donor E. coli culture. The recipient S. lavendulae culture was generated by inoculating 9 ml of TSB with 1 ml of frozen wild-type culture. Following overnight (16 hour) incubation at 29° C., the culture was homogenized by sonication and 2 ml of this culture was used to inoculate 18 ml of TSB. Following overnight growth at 29° C. and sonication treatment to homogenize the culture, a 1 ml inoculum was placed in 9 ml of TSB. This culture was grown for 3 hours, the mycelia were washed with TSB and resuspended in 2 ml of TSB to provide the stock recipient culture.

The donor and recipient cultures were mixed together in 9:1, 1:1, and 1:1/10 donor:recipient ratios, and 100 μl of the cell mixture was spread on AS1 plates (Baltz, 1980). The plates were incubated overnight at 29° C. and overlaid with 1 ml of water containing a suspension of 500 μg/ml each of thiostrepton, apramycin and nalidixic acid. For the pKC1139 control, only apramycin and nalidixic acid were overlaid, while for pDHS7702, 500 μg/ml of kanamycin was used instead of thiostrepton. S. lavendulae exconjugates appeared in approximately 11-13 days at a frequency ranging from 10⁻⁷-10⁻⁵. pKC1139 has a temperature-sensitive Streptomyces replication origin, which is unable to replicate at temperatures above 34° C. (Muth et al., 1989), while the S. lavendulae host grows well at 42° C. Thus, after propagating the conjugants at 39° C. for several generations, double crossover mutants were readily generated. Presence of plasmid was determined by transformation of E. coli DH5α with plasmid extracts from S. lavendulae transconjugants.

Double-crossover selection procedure. A single colony of S. lavendulae/pDHS2002 grown on R5T plates (50 μg/ml of thiostrepton and apramycin) was used to inoculate TSB broth containing 20 μg/ml of thiostrepton. After 72 hours of incubation at 39° C., 10⁻⁴, 10⁻⁵ and 10⁻⁶ diluted aliquots were used to inoculate R5T plates containing 50 μg/ml of thiostrepton. Following 48 hours of growth at 39° C., 84 colonies were picked randomly and each colony was patched out on separate 50 μg/ml of thiostrepton and 50 μg/ml of apramycin containing R5T plates. One of the 84 colonies displayed the double crossover phenotype of thiostrepton resistance and apramycin sensitivity. Integration of the tsr disrupted mitA gene and loss of plasmid pDHS2002 was confirmed by Southern hybridization analysis.

MitA K191A site-directed mutants (MV102) were selected by propagating MV100/pDHS2015 on R5T plates for two generations at 37° C. Colonies were replicated to plates containing 50 μg/ml of thiostrepton and plates without antibiotics. Of the 108 colonies replicated in the first round, one had the correct (thiostrepton sensitive) phenotype. To confirm the K191A mutation, the mitA gene was amplified from the chromosome with primers 1 and 4. Mutation of the conserved lysine codon (AAG) to an alanine codon (GCC) was verified with the same sequencing primers employed to confirm the correct construction of pDHS2015. The alanine codon was observed in both the forward and reverse sequence data.

Mutants for mitB (MM101) were selected as follows: S. lavendulae/pDHS7702 was propagated on R5T plates for five generations at 39° C. before single colonies were replicated on R5T plates as described above. Of the 300 colonies tested, 12 clones displayed the correct phenotype (kanamycin resistance and apramycin sensitivity). The genotype of selected mitB mutants was confirmed by Southern blot hybridization of S. lavendulae genomic DNA.

Analysis of MC production. All cultures intended for MC extraction were grown in Nishikohri media (Nishikohri and Fukui, 1975) for a period of 72 hours. In all cases a wild-type S. lavendulae culture was grown concurrently with the mutant cultures to provide a MC production reference point. A 72 hours, 50 ml culture (250 ml flask) of the MitA K191A MV102 mutant strain was supplemented with 125 μl of a 20 mg/ml solution of the sodium salt of AHBA (pH 7.05) at 24, 43 and 55 hours. In each case, the culture broth was separated from mycelia by centrifugation and then extracted three times with equal volumes of ethyl acetate. The ethyl acetate extracts were pooled and solvent was removed by vacuum to provide the crude broth extract. The preliminary screen for MC production involved thin layer chromatography (TLC) on silica gel plates (Whatman K6) eluted with 9:1 chloroform:methanol. Production of MC was monitored by HPLC (C₁₈ reverse phase column) using a gradient of 80% 50 mM Tris buffer (pH 7.1)/20% methanol to 40% 50 mM Tris buffer (pH 7.2)/60% methanol with the UV detector set to 363 nm.

Bioassay detection of MC was performed by loading a 1 cm disk with fractions eluting at the mitomycin retention time from HPLC injections of wild-type, MV100, pKC1139 vector control crude extracts and MC standards. The disks were placed on antibiotic media number 2 agar plates (Difco) with Bacillus subtilis spores added directly to the media. The plates were incubated overnight at 29° C. and examined for zones of inhibition. To confirm the production of MC by MV102 in the presence of exogenous AHBA the fraction eluting at the MC retention time was collected, dried down, desalted and submitted for desorption ionization mass spectrometric analysis on a Bio-Ion 20R DS-MS instrument (Applied Biosystems). The MC (M. W.=334)-sodium (M. W.=23) adduct peak, [M+Na]⁺=357, was diagnostic for the presence of MC in the AHBA supplemented culture.

Results

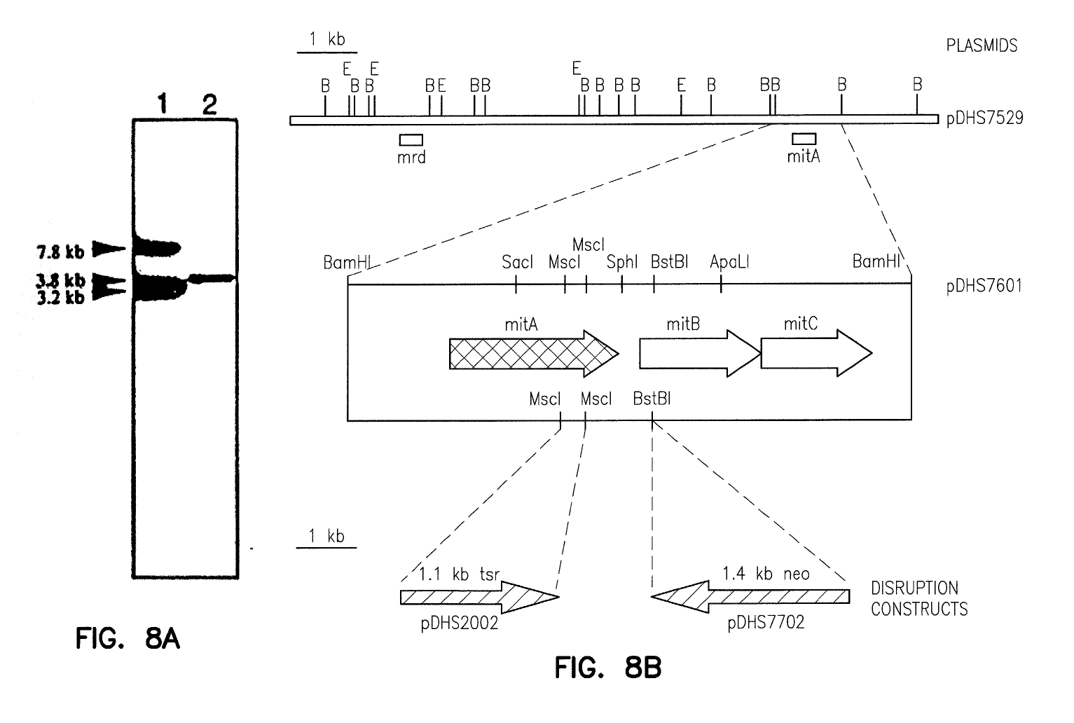

The mrd and ahbas genes are linked in the S. lavendulae genome. Southern blot analysis with the A. mediterranei AHBA synthase (rifK) gene probe (Kim et al., 1998) showed a single 3.8 kb band that hybridized with BamHI digested S. lavendulae genomic DNA (FIG. 8). Subsequently, a S. lavendulae genomic DNA library was constructed using the E. coli-Streptomyces shuttle cosmid pNJ1. Of the 5,000 colonies screened, 21 positive clones were identified with six of these hybridizing with the mrd gene probe (none hybridized with the mcr gene probe described in August et al., 1994). Restriction-enzyme mapping and reciprocal hybridization of the cosmid clones established that the mrd and S. mediterranei AHBA synthase homologous genes were about 20 kb apart in the S. lavendulae genome. The 3.8 kb BamHI fragment bearing a putative S. lavendulae AHBA synthase gene was subcloned and its nucleotide sequence determined.

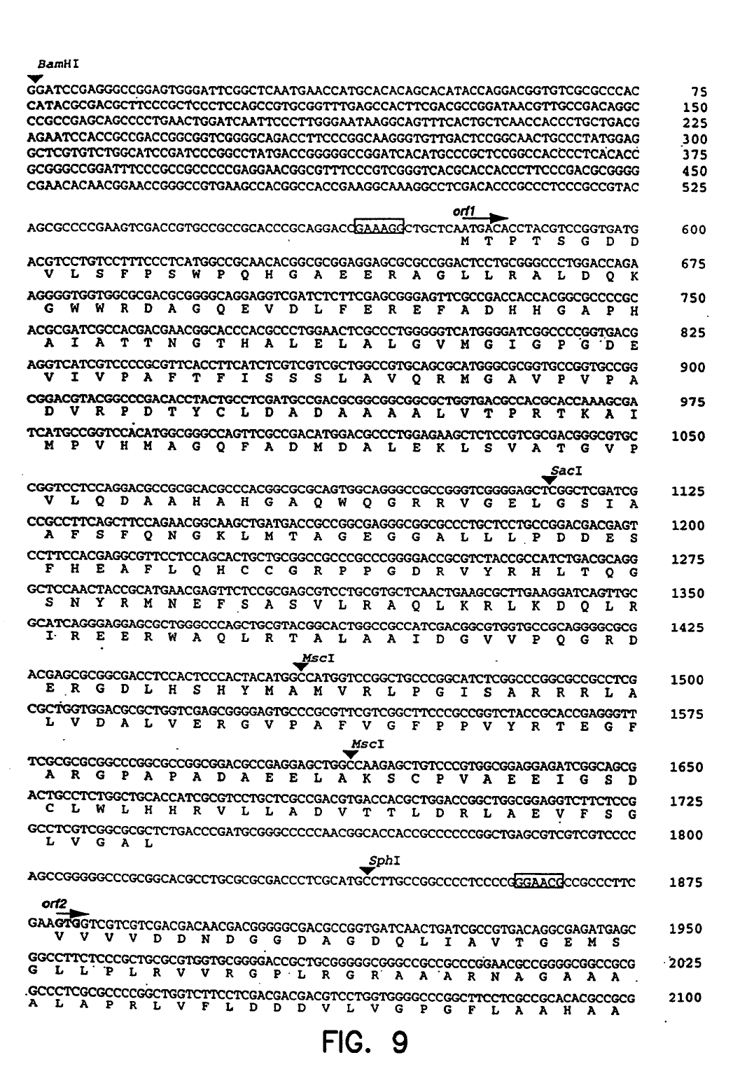

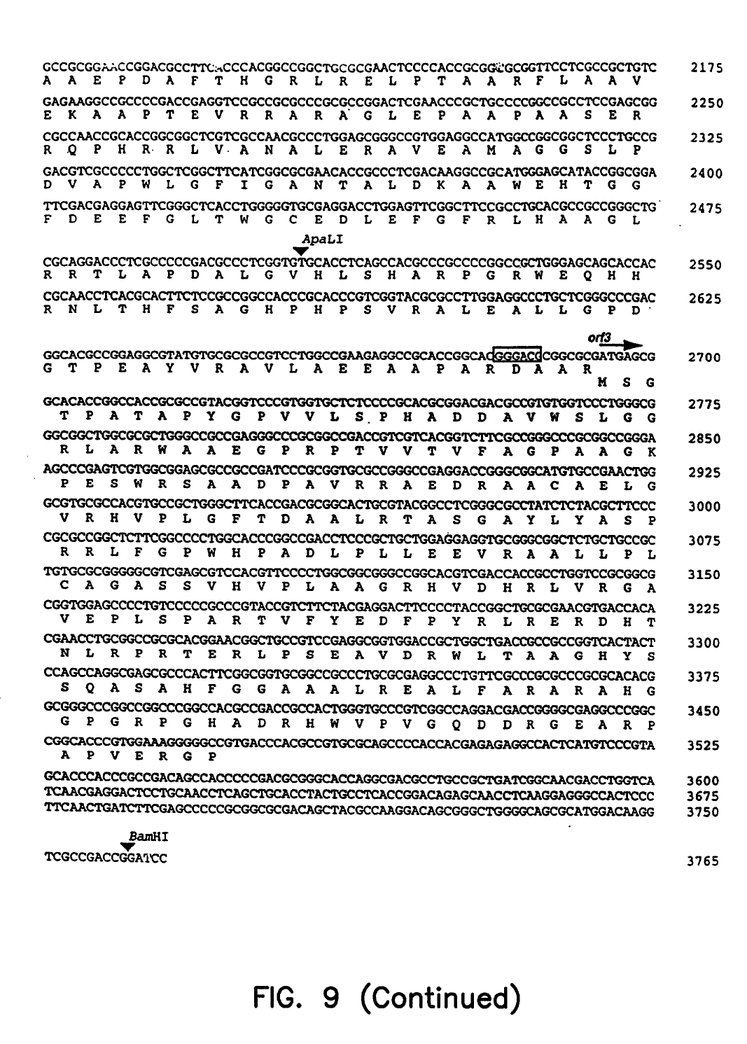

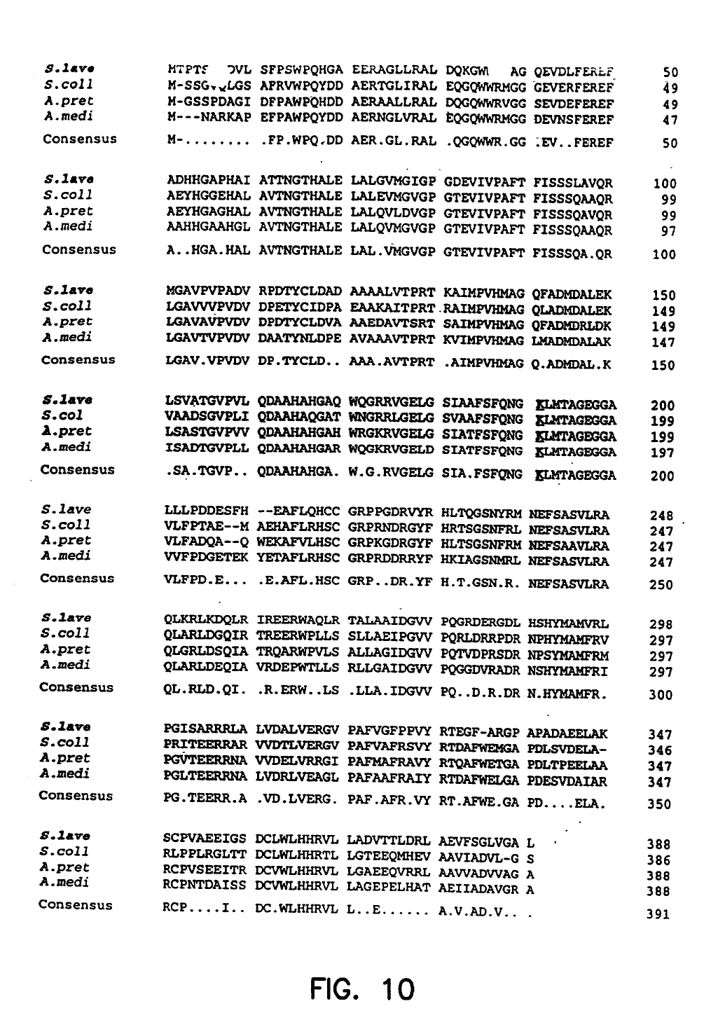

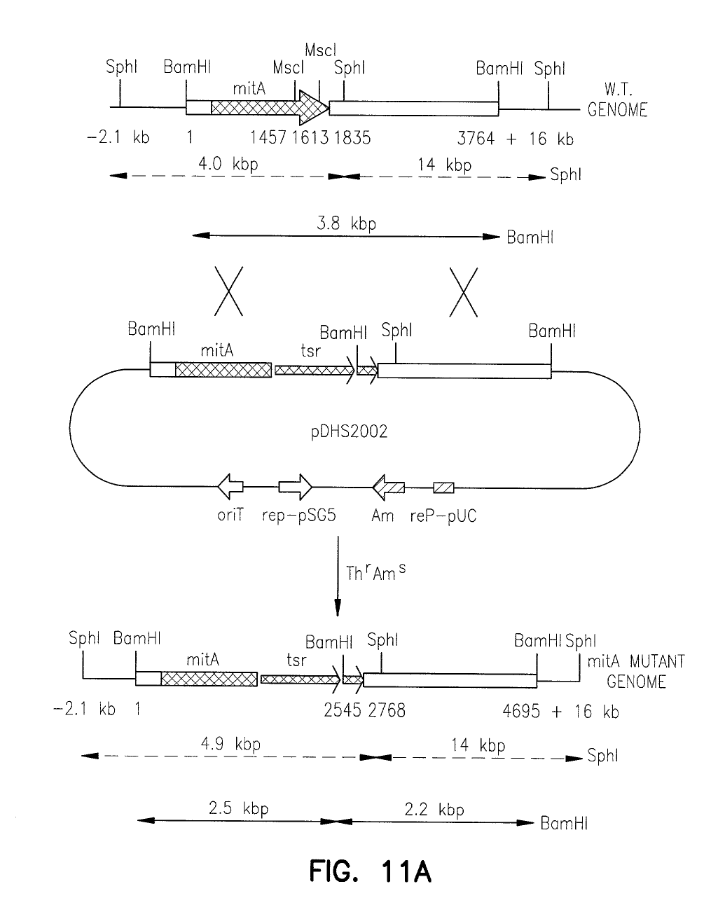

Three ORFs are identified within the 3.8 kb BamHI fragment. Three ORFs (mitA, mitB, mitC) were identified within the sequenced 3.8 kb BamHI fragment (FIGS. 8 and 9). mitA comprises 1164 nucleotides and starts from ATG (position 579 of the sequenced fragment) that is preceded by a potential ribosome binding site (RBS), GAAAGG (SEQ ID NO:91). The deduced product of the mitA gene encodes a hydrophilic protein of 388 amino acids with a predicted Mᵣ of 41,949 Da and a calculated pI of 5.62. A BLAST (Altschul et al., 1990) search showed that the predicted MitA protein has high sequence similarity (about 71% identity, 80% similarity) with AHBA synthases (AHBASs), both from the rifamycin producer A. mediterranei (Kim et al., 1998) and other ansamycin-producing actinomycetes, including Actinosynnema pretiosum (ansamitocin) and Streptomyces collinus (naphthomycin A and ansatrienin) (FIG. 10). A conserved pyridoxal phosphate (PLP) coenzyme binding motif (GX₃DX₇AX₈EDX₁₄GX₁₃KX₄₋₅geGGX₁₉G) (SEQ ID NO:92) including the conserved lysine residue can also be found in these four proteins (Piepersberg, 1994).

The mitB gene is predicted to start at a GTG (position 1879) that is preceded by a presumed RBS (GGAACG) (SEQ ID NO:93). This gene encodes a 272 amino acid protein with a deduced Mᵣ or 28,648 Da and a deduced pI of 6.06. Database sequence homology searches revealed that the product of mitB shows local sequence similarity with a group of O-glycosyltransferases involved in polysaccharide biosynthesis. One segment of 70 amino acid residues at the N-terminus of MitB has 43% similarity (36% identity) with the two glycosyltransferases SpsL and SpsQ from Sphingomonas S88, and ExoO form Rhizobium meliloti involved in polysaccharide (S88) and succinoglycan biosynthesis, respectively (Becker et al., 1963). Another 60 amino acid residues located at the C-terminus displayed 30% identity with UDP-GalNAc:polypeptide N-acetylgalactosaminyltransferase from Mus musculus and Homo sapiens (Bennett et al., 1996).

The third ORF, mitC, starts from the ATG at position 2694, which is coupled to the stop codon TGA of mitB and encodes a putative protein of 260 amino acids with a molecular mass of 27,817 Da and a pI of 10.45. Database searches with the deduced protein product showed significant similarity over the first 90 amino acids (38% identity, 40% similarity) with the ImbE gene product (unknown function ) from Mycobacterium leprae (U15183).

Insertional disruption of the mita and mitB genes in Streptomyces lavendulae. To test the dependence of functional mitA and mitB genes for MC biosynthesis, gene disruption constructs were generated for subsequent isolation of the corresponding S. lavendulae isogenic mutant strains.

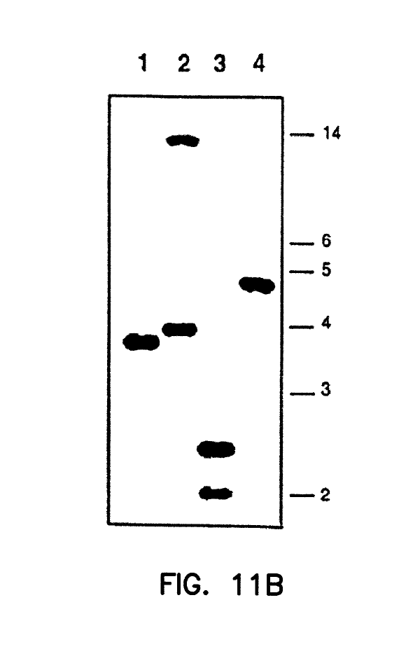

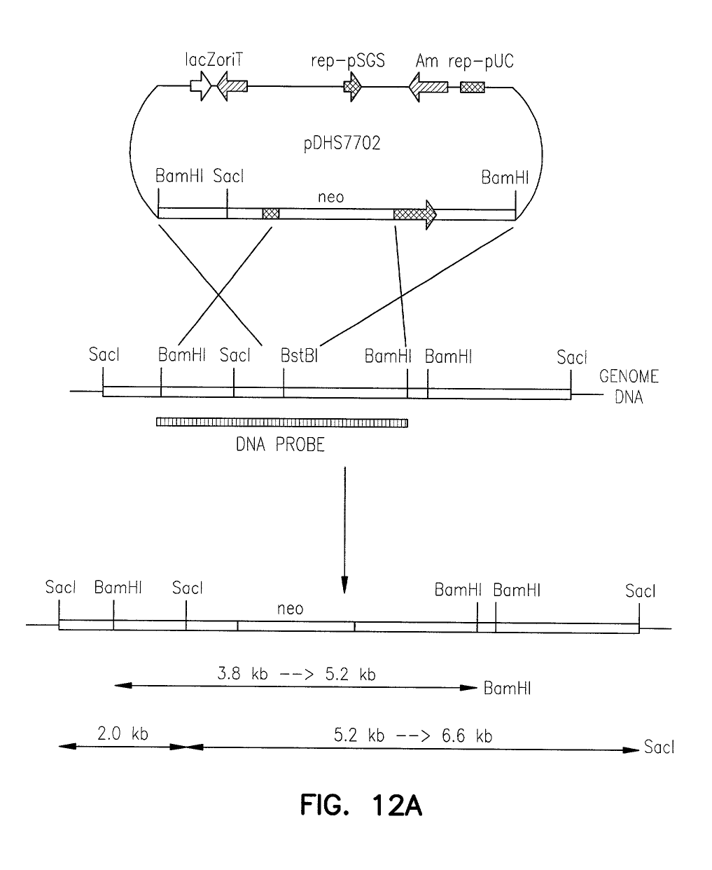

The mitA disruption construct was made by replacing a 155 bp fragment between the two MscI sites (located at the C-terminus of the mitA gene in pDHS7601) with the 1.1 kb SmaI-BamHI fragment containing a thiostrepton resistance gene from pDHS5000 (FIG. 11A). This replacement regenerated a BamHI site at the junction and the resulting construct was then subcloned into the E. coli-Streptomyces conjugative shuttle plasmid pKC1139, followed by conjugation into S. lavendulae. A double crossover mutant strain (MV100) was selected based on the expected phenotype (thiostrepton resistant, apramycin sensitive), and further confirmed by Southern blot hybridization. Genomic DNA from wild-type S. lavendulae and MV100 was digested with BamHI and SphI, and hybridized with the 4.9 kb EcoRI-HindIII tsr-disrupted mitA fragment from pDHS2001. As expected, the 4.0 kb SphI hybridized band in the wild-type strain was shifted to 4.9 kb in MV100, whereas the 3.8 kb BamHI hybridization and in the wild-type was converted to two bands (2.2 kb and 2.5 kb) in the mutant (FIG. 11B).

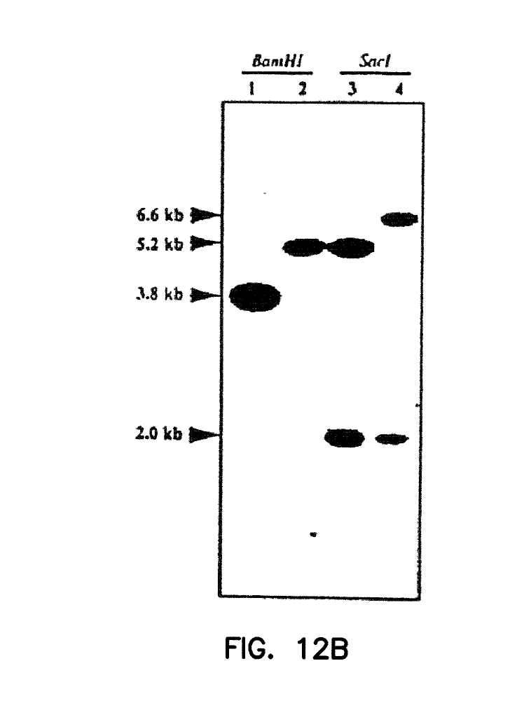

The mitB gene was disrupted by inserting a neomycin resistance gene (aphII) into the BstBI site (located at the 5′-end of mitB) (FIG. 12A). Transconjugants were selected on kanamycin/apramycin plates, and a double crossover mutant strain (MM101) was identified with a kanamycin-resistant, apramycin-sensitive phenotype and subsequently confirmed by Southern blot hybridization. As expected, the 3.8 kb BamHI hybridization band in wild-type S. lavendulae was shifted to 5.2 kb in MM101, whereas a 5.2 kb SacI hybridization band was shifted to 6.6 kb (FIG. 12B).

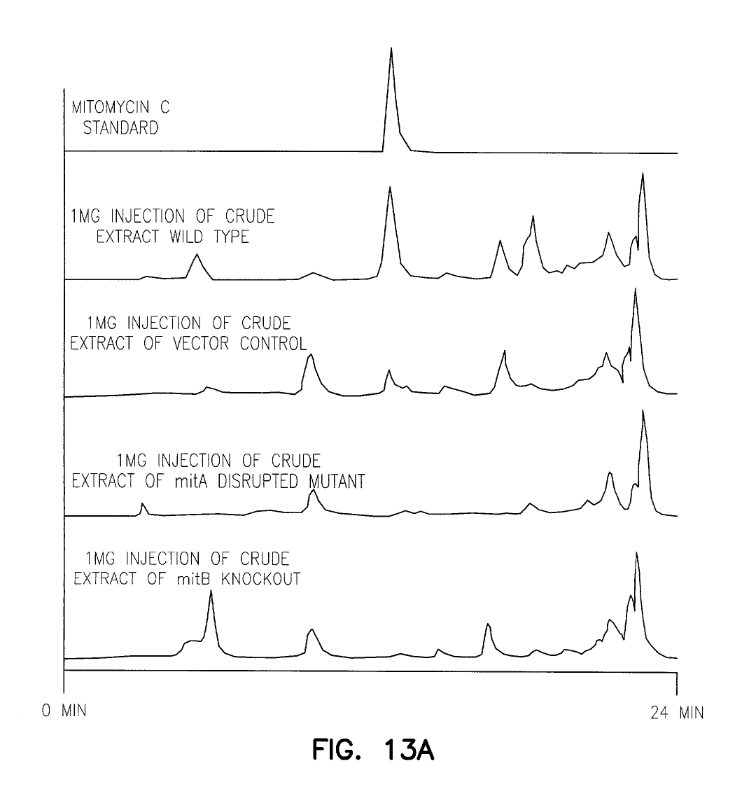

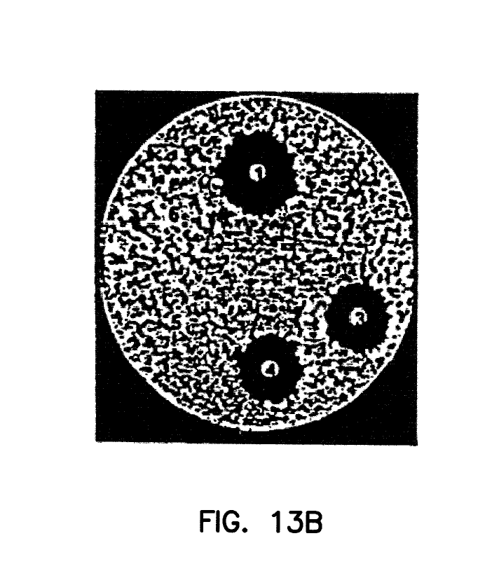

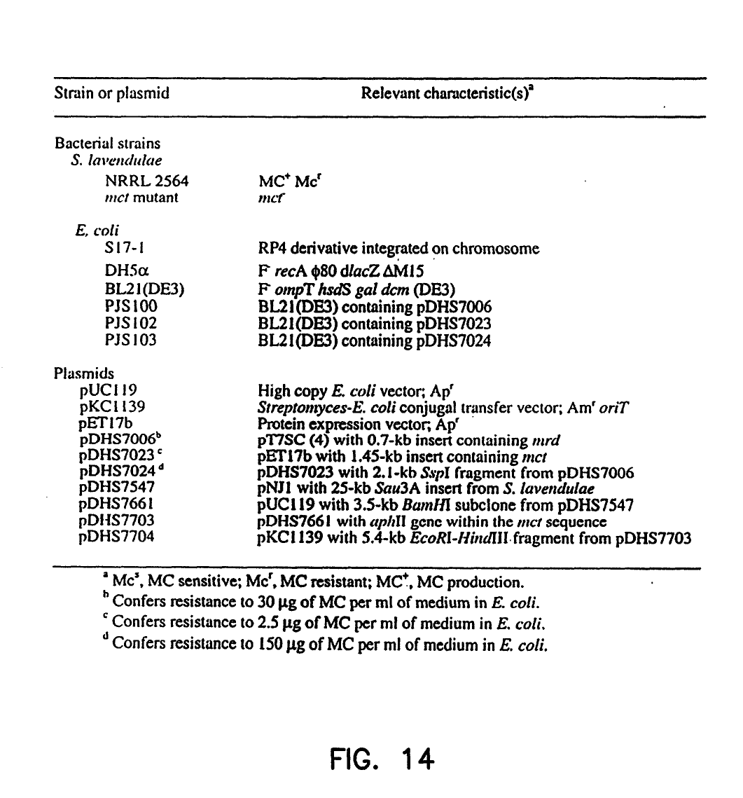

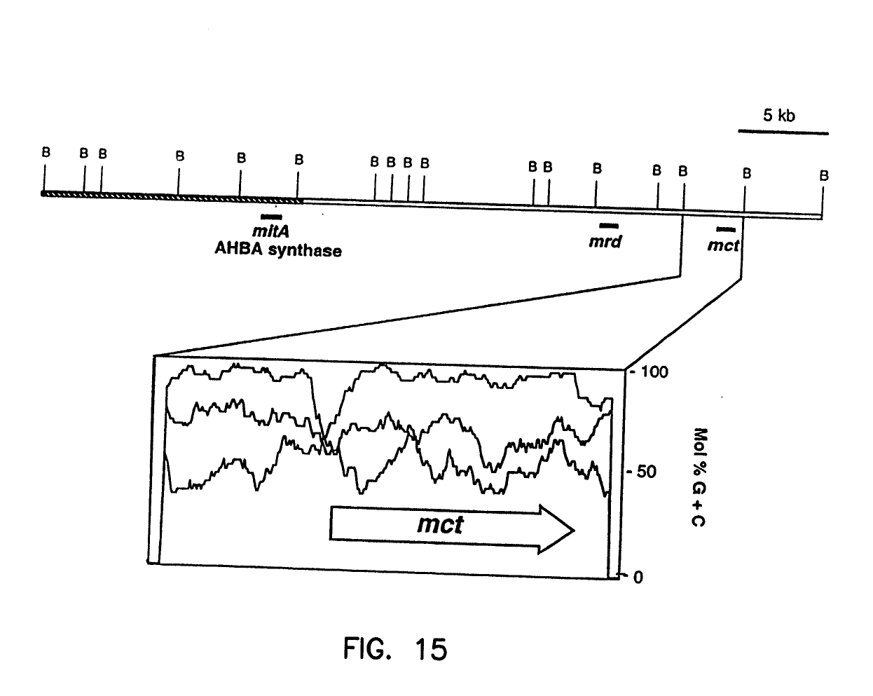

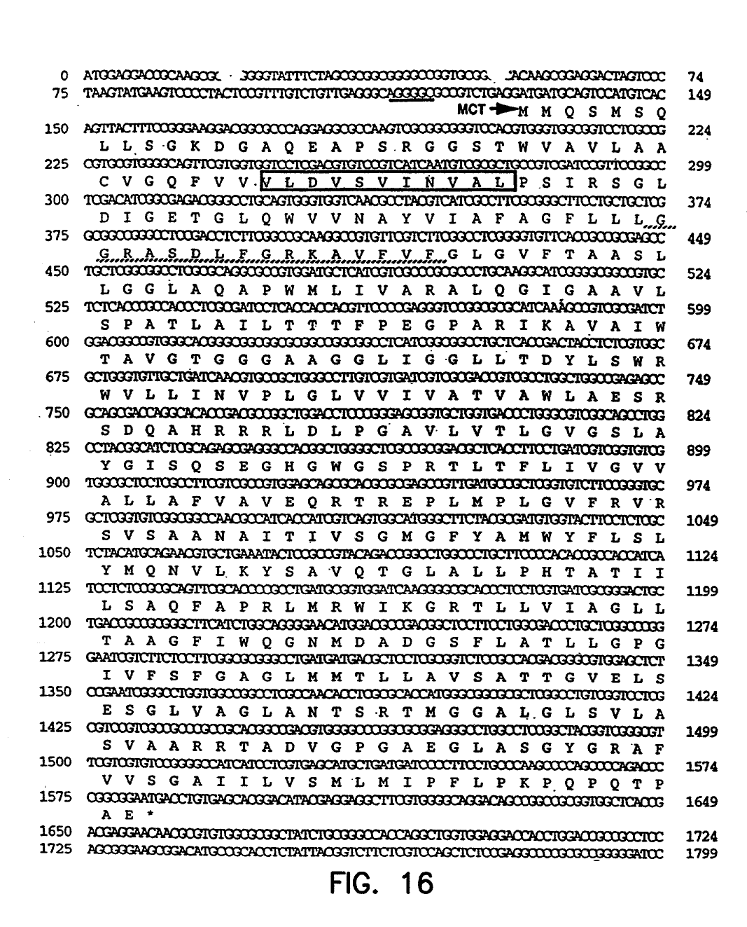

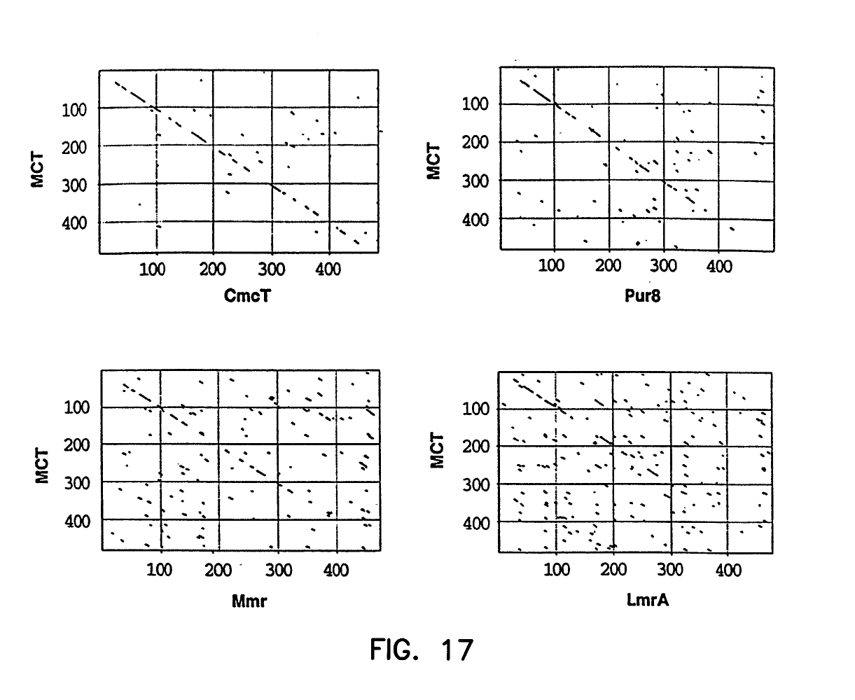

mitA and mitB disrupted strains (MV100, MM101) are blocked in MC biosynthesis. The growth characteristics and morphology of MV100 and MM1001 in liquid media and on agar plates was identical to wild-type S. lavendulae. HPLC was used to quantify production of MC in MV100 and MM101 (FIG. 13A), and culture extracts were used in a biological assay to test for presence of the drug (FIG. 13B). Injection of one mg of wild-type S. lavendulae culture extract gave a peak in the HPLC that eluted with the same retention time as the MC standard. Upon injection of one mg of culture extract from the mitA or mitB disrupted strains (MV100, MM101) no MC peak was observed. To corroborate the lack of production of MC, the HPLC eluant obtained from the MV100 culture extracts was collected over the retention time range determined for MC. This eluant completely lacked biological activity against Bacillus subtilis (the MC target strain) while the fraction collected from the same retention time region of wild-type S. lavendulae and the vector control strain culture extracts showed substantial levels of biological activity (FIG. 13B).

It is important to note that the presence of the vector pKC1139 in S. lavendulae reduced the percentage of MC in the total crude extract while simultaneously increasing the total amount of material extractable by ethyl acetate. The combination of these two effects reduces the absolute amount of MC by approximately 25% in the vector control culture crude extract compared to the wild-type crude extract.

Exogenous AHBA can restore MC, production in the MC-deficient MitA K191A mutant. Although complementation of MV100 (mitA insertional disruptant) was attempted by providing exogenous 3-amino-5-hydroxybenzoic acid in the culture medium, MC production was not restored as measured by HPLC or biological assay. A polar effect on genes downstream of tsr-disrupted mitA in MV100 appeared likely since supplying mitA in trans on a medium copy number plasmid (MV103) also failed to restore MC production. Therefore, site-directed mutagenesis was employed to generate a MitA K191A mutant resulting in strain MV102. Kim et al. (1998) had demonstrated that the AHBA synthase from A. mediterranei is PLP dependent and catalyzes the aromatization of 5-deoxy-5-amino-3-dehydroshikimic acid (aminoDHS). Thus, the nitrogen of the conserved lysine 191 is supposed to form a Schiff base with the PLP cofactor. Replacement of lysine 191 with alanine prevents binding of the cofactor and eliminates enzymatic activity. Replacement of the AGG encoding lysine 191 in wild-type S. lavendulae with a GCC codon in MV102 was confirmed by nucleotide sequence analysis. As expected, MV102 did not produce MC, however, when the culture medium was supplemented with exogenous AHBA, MC production was restored as determined by MS ([M+Na]⁺=357), HPLC and TLC analysis (Table 2).

TABLE 2
Complementation results with (+) or without (−) AHBA.

| | |
|---|---|
| S. lavendulae | MC production |

| strains | −AHBA | +AHBA |
|---|---|---|
| Wild-type | + | + |
| MV100 | − | − |
| MV103 | − | − |
| MV102 | − | + |

Discussion

An effective strategy for the identification of natural product biosynthetic gene clusters in actinomycetes has included cloning of antibiotic resistance genes followed by investigation of adjacent DNA for the presence of structural and regulatory genes (Butler et al., 1989, Donadio et al., 1991; Motamedi and Hutchinson, 1987; Vara et al., 1985). Although linkage of antibiotic resistance and biosynthetic genes appears to be a general feature in prokaryotes, a growing number of examples involve the existence of multiple resistance loci that may be linked or unlinked to the biosynthetic gene cluster (Vara et al., 1985; Seno and Baltz, 1989; Smith et al., 1995). The identification and characterization of two genetically unlinked resistance loci (August et al, 1994; Sheldon et al., 1997) for MC created a dilemma for mounting an effective search for the MC biosynthetic gene cluster. However, the use of the AHBA synthase gene from A. mediterranei provided an effective probe to identify cosmid clones bearing a linked MC resistance gene. Thus, the isolation of several cosmid clones form an S. lavendulae genomic DNA library that hybridized to both the A. mediterranei AHBA synthase gene and the S. lavendulae mrd gene indicated that the MC biosynthetic gene cluster resided on DNA adjacent to mrd. DNA sequence analysis of the 3.8 kb BamHI fragment revealed three ORFs whose deduced protein sequences corresponded to an AHBA synthase, a glycosyltransferase, and a lmbE-like product.

As determined by precursor feeding experiments, the mitosane core is formed through the condensation of AHBA and D-glucosamine (Homemann, 1981). AHBA is thought to be derived from the ammoniated shikimate pathway from PEP and E4P, in which the last step from aminoDHS to ABBA is catalyzed by AHBA synthase (FIG. 7) (Kim et al., 1996; Kim et al., 1998). Meanwhile, the reaction of attaching an activated sugar residue to a core compound is usually catalyzed by a group of enzymes called glycosyltransferases as specified by macrolide, glycopeptide antibiotic and polysaccharide biosynthesis (Kahler et al., 1996; Otten et al., 1995b; Solenberg et al., 1997; Yamazaki et al., 1996). In principle, the condensation of AHBA with D-glucosamine can be initiated in two different ways (FIG. 7). One would involve the formation of the C₈ₐ-C₉ bond by an electrophilic aromatic alkylation or acylation. A second possibility would be formation of a Schiff base between the nitrogen of AHBA and the D-glucosamine C1 aldehyde, followed by ring closure at C₈ₐ-C₉. In either case, a C- or N- instead of O-glycosyltransferase is expected. Although previously described glycosyltransferases display a high degree of sequence divergence (Yamaazaki et al., 1996), the mechanistic similarity with O-glycosyl transfer may suggest that mitB encodes a N-glycosyltransferase that initiates the formation of the mitosane system by linking glucosamine to AHBA. The mitA and mitB genes and their corresponding products are likely candidates to mediate formation of AHBA and the mitosane ring system, respectively. However, the possible function of the lmbE-like protein remains unclear, since its current role within lincomycin biosynthetic pathway of S. lincolnensis is not known (Peschke, 1995).

The involvement of AHBA synthase (mitA) and the putative glycosyltransferase (mitB) in MC biosynthesis was established by gene disruption to create mutants blocked in MC biosynthesis. This required development of a method to introduce DNA into S. lavendulae NRRL 2564 since the strain remains refractory to traditional Streptomyces protoplast and electroporation-mediated transformation procedures. Other such refractory strains include, but are not limited to, ATCC 27422. The modified Bierman protocol (Bierman et al., 1992) was used to affect efficient conjugative transfer into S. lavendulae using the E. coli-Streptomyces shuttle plasmid pKC1139. This result is significant because it permits the development of an effective system for analyzing in detail the genes involved in mitomycin biosynthesis.

The function of mitA was probed by providing strains MV100 and MV102 with exogenous 3-amino-5-hydroxybenzoic acid in the culture medium. Despite repeated attempts to complement MV100, MC production was not restored as measured by HPLC or biological assay. It is believed that insertion of the tsr gene into mitA resulted in disruption of biosynthetic genes immediately downstream, since supplying mitA in trans on a medium copy number plasmid also failed to restore MC production to MV100. This putative polar effect was eliminated by generating the MitA K191A mutant strain MV102. Providing exogenous 3-amino-5-hydroxybenzoic acid to this mutant strain of S. lavendulae restored production of MC as shown by TLC, HPLC and mass spectrometry. When MV102 was grown in the absence of AHBA, there was no detectable production of MC. The ability of 3-amino-5-hydroxybenzoic acid to complement the mutant MitA protein further supports the function of MitA as an AHBA synthase as indicated by the database protein sequence alignment and previous studies on rifK (Kim et al., 1998).

### EXAMPLE 3

### Mitomycin Resistance in Streptomyces lavendulae Includes a Drug-Binding Protein-Dependent Export System

As a prodrug, MC is unreactive until chemical or enzymatic reduction renders the molecule a highly effective alkylating agent (Iyer and Szybalski, 1964). The molecular basis of MC bioactivity derives mainly from its propensity to covalently interact with DNA at 5′-CpG sequences, causing lethal intra- and inter-strand crosslinks as well as monofunctional alkylation (Tomasz, 1995).

S. lavendulae encounters a daunting challenge in avoiding potentially lethal MC-mediated crosslinks since it has a chromosomal G+C content of over 70%, which translates into at least one million potential drug target sites per cell. Indeed, two genetic loci that mediate mitomycin resistance have been reported in this organism. One locus (mcr) encodes a protein (MCRA) that catalyzes oxidation of the reduced, bioactivated species of MC via a redox relay mechanism (August et al., 1994; Johnson et al., 1997). The second locus (mrd) encodes MRD that functions to sequester the prodrug by a specific mitomycin-binding protein (Sheldon et al., 1997). A paradox of current knowledge regarding mitomycin resistance has been the lack of a clear mechanism for drug transport. Indeed, the observed stoichiometry suggests that it would be ineffective for S. lavendulae to utilize MRD as a solo mechanism for cellular self-protection. Pathogenic bacteria (Nikaido, 1994), and antibiotic-producing microorganisms (Cundliffe, 1992; Mendez and Salas, 1998), employ export of toxic compounds as a means of resistance.

Materials and Methods

Bacterial strains, culture conditions, and media. E. coli DH5α used as a host for generation of double-stranded plasmid DNA, was grown at 37° C. on LB medium. E. coli BL21 (DE3), used as host for protein expression, was grown at 37° C. in NZCYM medium (Sambrook et al., 1989). S. lavendulae NRRL 2564 was grown on YEME medium (Hopwood et al., 1985) at 30° C. for preparation of genomic DNA.

DNA preparation and amplification. S. lavendulae genomic DNA was isolated by the lysozyme-2×Kirby mix method (Hopwood et al., 1988). General DNA manipulation was performed as described previously (August et al., 1994). Oligonucleotides for PCR and sequencing were obtained from Gibco BRL. PCR amplifications were carried out using a Hybaid thermal cycler (Hybaid Ltd., Teddington, U.K.).

Cloning and sequencing of mct. A S. lavendulae NRRL 2564 genomic DNA library was constructed in the cosmid vector pNJ1 (Tuan et al., 1990) as previously described (August et al., 1994). The insert DNA of a cosmid clone containing sequences flanking mrd was digested with BamHI and subcloned into the BamHI site of pUC119. Using exonuclease III (Erase-A-Base kit, Promega, Madison, Wis.), a set of nested deletion clones was generated and both strands of the insert DNA were sequenced by the dideoxy chain termination method using the ABI Prism kit (PE Applied Biosystems) in coordination with an ABI 373 automated sequencer. 10% DMSO was added to the reactions to reduce compressions Sequence data was analyzed using the GeneWorks (Oxford Molecular) software package. Deduced amino acid sequence data were compared to the available databases using the BLAST program of the Genetics Computer Group version 9.0 software (Oxford Molecular Group). The mct gene has been deposited in the GenBank database under Accession No. AF120930.

Construction of the mct mutant strain of S. lavendulae. The mct disruption vector pDHS7704 was constructed as follows. pDHS7661 was digested with EcoRI, blunt-ended, and ligated with the 1.4 kb neomycin resistance gene fragment from pFD666 (ApaLI-HindIII digestion, blunt-ended) (Ames, 1986). The 5.4 kb EcoRI-HindIII fragment from the resulting construct (pDHS7703) was subcloned into pKC1139 to create pDHS7704, and conjugated into S. Iavendulae according to Bierman et al. (1992). A mct double crossover mutant was selected after propagating transconjugants on R5T plates for five generations at 39° C. Kanamycin-resistant and apramycin-sensitive colonies were further tested by Southern blot to confirm the desired double crossover genotype.

Construction of mct expression plasmid. For the construction of the E. coli expression plasmid NdeI and HindIII sites were introduced at the translational start codon and downstream of the translational stop codon of mct, respectively. The primers used for PCR were 5′-GGGAATTCCATATGATGCAGTCCATGTCAC-3′ (SEQ ID NO:94) and 5′-GGGAATTCAAGCTTTCATTCCGCCGGGGTC-3′ (SEQ ID NO:95). The PCR was carried out using 2.5 U of Taq polymerase, 0.4 μg of each primer, 1 μg of pDHS7661 DNA as template, 10 mM each of dATP-dGTP-dCTP-dTTP, 1.5 mM MgCl₂, and 10 μl of 10×Promega PCR buffer in a total volume of 100 μl . Amplification was achieved with 30 cycles of denaturation at 94° C. for 30 seconds, annealing at 37° C. for 1 minute, and extension at 70° C. for 2 minutes. The 1.45 kb PCR product was recovered by 0.8% agarose gel electrophoresis, digested with NdeI-HindIII and ligated into the T7 expression plasmid pET17b (Novagen), which had been similarly cut with EcoRI-HindIII, to give pDHS7023. pDHS7023 was introduced by transformation into E. coli BL21(DE3) to provide strain PJS102.

Construction of mct-mrd co-expression plasmid. From plasmid pDHS7006 (Sheldon et al., 1997), a 2.1 kb SspI fragment was isolated. The fragment contained the mrd gene under the control of the T7 promoter, including transcriptional terminator sequences (rrnB T1) upstream and downstream of mrd. The fragment was ligated into the MC-translocase construct pDHS7023, which had been cut with MscI, to give pDHS7024. pDHS7024 was introduced by transformation into E. coli BL21(DE3) to result in strain PJS103.

MC resistance phenotppe of E. coli. To analyze resistance conferred by the expression of the MC-translocase in E. coli, 10 μl of strain PJS102 was spread on LB agar medium containing 100 μg/ml of ampicillin, IPTG to a final concentration of 1.0 mM, and various concentrations of MC. The cultures were grown overnight at 37° C. and colony-forming units (CFUs) were determined. Similarly, the MC resistance phenotype of strain PJS103 (mcr-mrd co-expression strain) was quantified.

[³H]-MC uptake assay of strains PJS102 and PJS103. [³H]-MC was obtained from Kyowa Hakko Kogyo, Ltd. Uptake studies were performed for whole cells of PJS100, PJS102, PJS103 and E. coli BL21 (DE3)::pT7SC and pET17b. PJS100, PJS102, and PJS103 as well as vectoronly cultures were cultured (37° C.) in 5 ml of NCZYM medium with IPTG added to a final concentration of 1 mM (at approximately 3 hours growth). At 9 hours (late exponential phase), cells were harvested by centrifugation and resuspended in 1 ml NCZYM broth (5×concentration). The concentrated suspension of late-exponential growth phase cells was exposed to [³H]-MC (59 Ci/mmol) at a final concentration of 0.022 μg/ml (0.0655 nmol). Aliquots (100 μl) were removed at frequent intervals, placed on 1.2 μM GF/C filters (Whatman International, Maidstone, U.K.) and washed once with 6 ml of 0.85% NaCl poured over the filters under vacuum pressure. Additional aliquots were simultaneously removed for determination of protein content (protein assay kit, Bio-Rad Laboratories, Richmond, Calif.). Radioactivity on the filters was quantified using a Beclkan LS7000 scintillation counter. Results were expressed as nanograins of mitomycin per milligram of cell protein.

Results

A gene encoding a transmembrane protein is physically linked to mrd. DNA sequence analysis of a cosmid clone containing the mrd locus, a previously characterized MC resistance determinant (Sheldon et al., 1997), identified an open reading frame (ORF) encoding a polypeptide predicted to be highly hydrophobic that shows similarity to a variety of antibiotic export proteins in drug-producing actinomycetes. Significantly, the gene (mct, SEQ ID NO:72) encoding the putative mitomycin exporter (MC-translocase; MCT) protein is located within 5 kb of mrd (SEQ ID NO:64) and is physically linked to the mitomycin biosynthetic gene cluster (FIG. 15).

Sequence analysis of the mct locus. Nucleotide sequence analysis of cosmid clone pDHS7547 revealed an ORF predicted to start with the ATG codon at position 132 and end with the TGA codon at nucleotide 1587 (FIG. 16), resulting in a 484 amino acid polypeptide with a predicted molecular weight of 50,023 daltons. Comparison of the deduced amino acid sequence of the mct gene with proteins in the available databases revealed significant similarity to several integral membrane proteins that confer drug resistance. These include the CmcT protein from the cephamycin producer, Nocardia lactamdurans (Coque et al., 1993), the Pur8 protein from the puromycin producer, Streptomyces alboniger (Tercero et al, 1993), the Mmr protein from the methylenomycin producer, Streptomyces coelicolor (Neal and Chater, 1987), and the LmrA protein from the lincomycin producer, Streptomyces lincolnensis (Zhang et al., 1992). The similarities of the mct gene product and related proteins extend over the entire sequences, with the highest levels of similarity found within the amino-terminal regions (FIG. 17).

Within the N-terminal regions of several antibiotic efflux proteins, including Mmr and LmrA, several highly conserved structural motifs have been noted. The β-turn motif (VxGxLxDxxGRKxxxL), found within the highly conserved cytoplasmic loop sequence separating transmembrane domains two and three of most eukaryotic and prokaryotic transport proteins, is clearly evident in MCT at positions 79-95 (FIG. 16). A motif (LDxTVxNVALP) found at the end of transmembrane domain one, specific to the 14 transmembrane segment family (Paulson and Skurray (1993)) is present in MCT at positions 41-51 (FIG. 16). In addition, several other invariant motifs are apparent in the MCT sequence.

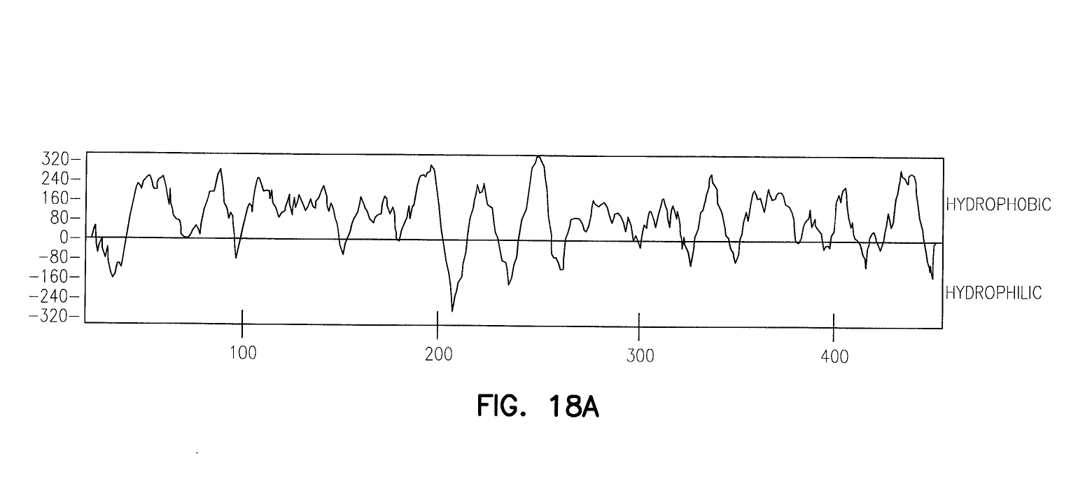

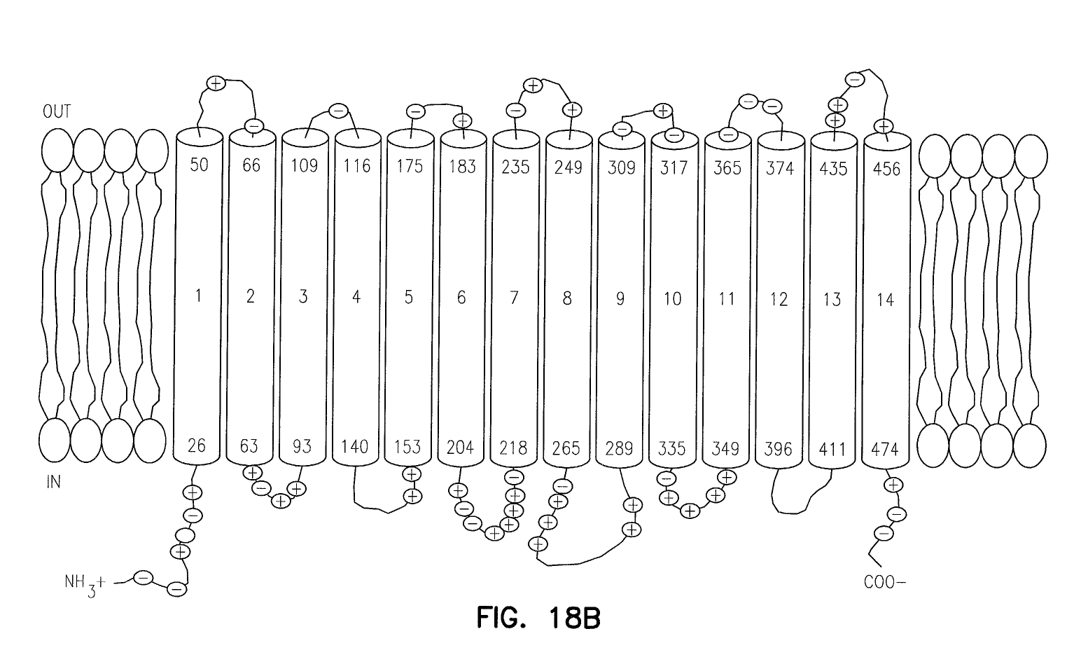

Transmembrane proteins that mediate resistance to antibiotics and antiseptics by active efflux are highly related, usually containing 12 or 14 transmembrane regions. Notably, the actinomycete drug transport proteins that share homology with MCT appear to contain 14 transmembrane spanning regions and constitute a family of drug resistance translocases. Utilizing the membrane structure and topology program MEMSAT (University College, London), and hydropathy analyses based on the algorithm of Kyte and Doolittle (1982), a prediction of 14 transmembrane spanning domains was made for the deduced amino acid sequence of MCT (FIG. 18).

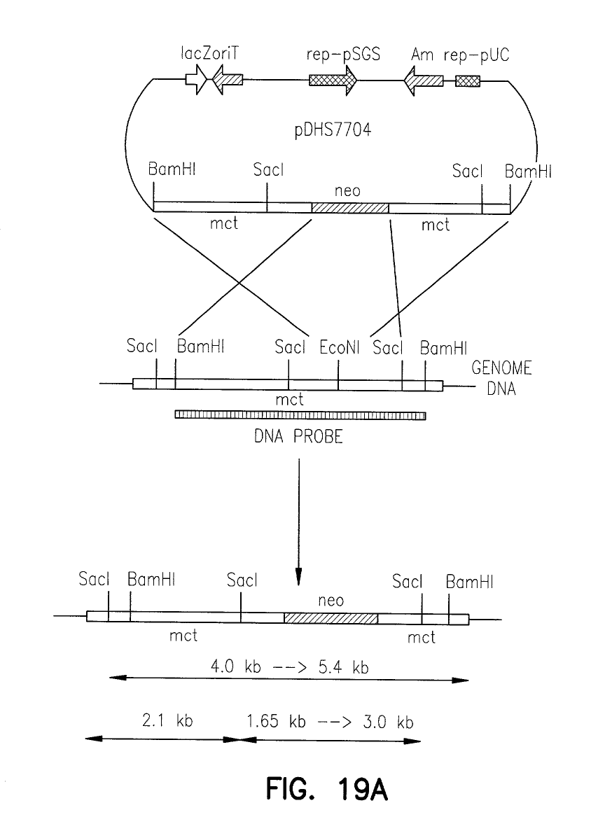

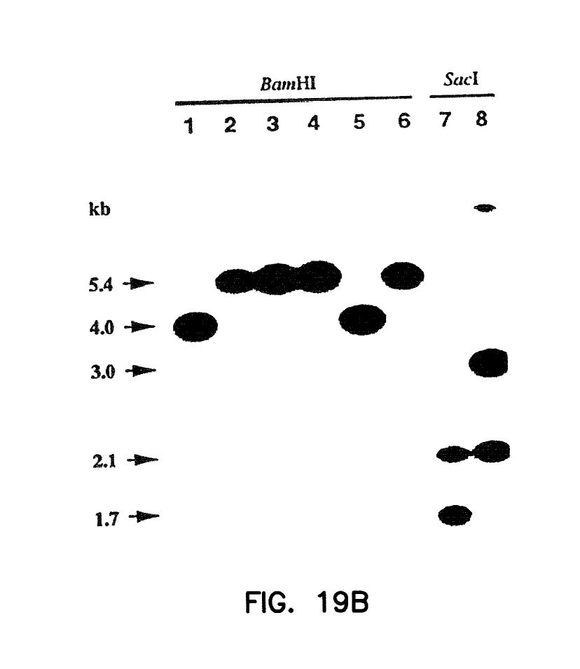

Inactivation of mct results in greater sensitivity to MC. To establish a physiological role for MCT in S. lavendulae, the corresponding gene (mct) was inactivated by insertion of the aphII gene from transposon Tn5 to give pDHS7704. After conjugal transfer of pDHS7704 from E. coli to S. lavendulae and growth of the transconjugants under selective conditions, targeted replacement of native mct was achieved by double crossover homologous recombination. Gene disruption was confirmed by Southern blot hybridization of total DNA from the S. lavendulae wild-type and mutant with a DNA probe that included the mct locus. Analytical digests of the genomic DNA resulted in detection of the predicted band shifts in the mutant and wild-type strains (FIG. 19). The S. lavendulae mct disruption mutant strain (MM105) exhibited an approximately 25-fold reduction in resistance to MC when exposed to 100 μg of MC per ml of medium (Table 3). In media lacking MC, the growth kinetics of the strain MM105 was comparable to the wild-type S. lavendulae strain.

TABLE 3
Resistance of S. lavendulae strains to
varying concentrations of MC

| | Plate count CFU/ml | |
|---|---|---|
| | Strain | |
| Concentration | | S. lavendulae mct mutant |
| of MC (μg/ml) | S. lavendulae wild-type | (MM 105) |
| 10 | >10⁷ | >10⁷ |
| 20 | >10⁷ | >10⁷ |
| 40 | 5.3 × 10³ | 2.6 × 10³ |
| 80 | 2.6 × 10³ | 2.4 × 10² |
| 100 | 2.0 × 10³ | 8.0 × 10¹ |

Expression of mct in E. coli. To investigate further the function of mct, heterologous expression of the gene in E. coli was pursued. mct was amplified by PCR and cloned into the protein expression vector pET17b to give pDHS7023. pDHS7023 was then introduced into E. coli BL21(DE3) to give strain PJS102. After disruption of the cells by sonication, MCT was found to be associated mainly with the membrane fraction of the cell lysate, as expected for an integral membrane protein. To determine if strain PJS102 was resistant to MC, cultures were grown up and plated on agar medium containing various concentrations of MC. Significantly, IPTG-induced cultures of PJS102 exhibited resistance to MC at drug concentrations 5-fold greater than those for E. coli BL21(DE3) containing vector alone (Table 4).

TABLE 4
MC resistance of mct, mrd expressing E. coli strains

| | Plate count CFU/ml | | | |
|---|---|---|---|---|
| | Strain | | | |
| Concentration | BL21 (DE3):: | | | |
| of MC (μg/ml) | pET17b | PJS100 | PJS102 | PJS103 |
| 0.0 | >10⁷ | >10⁷ | >10⁷ | >10⁷ |
| 0.01 | >10⁷ | >10⁷ | >10⁷ | >10⁷ |
| 0.1 | 7.3 × 10³ | >10⁷ | >10⁷ | >10⁷ |
| 0.5 | 3.2 × 10² | >10⁷ | 2.1 × 10⁵ | >10⁷ |
| 1.0 | 0.0 | 3.3 × 10⁶ | 5.9 × 10⁴ | >10⁷ |
| 2.5 | — | NA^{a} | 2.0 × 10² | >10⁷ |
| 5.0 | — | 2.7 × 10⁶ | 0.0 | >10⁷ |
| 10 | — | 6.1 × 10⁵ | — | >10⁷ |
| 20 | — | 2.5 × 10⁵ | — | >10⁷ |
| 30 | — | 5.0 × 10² | — | >10⁷ |
| 60 | — | 0.0 | — | >10⁷ |
| 80 | — | — | — | 1.4 × 10⁵ |
| 100 | — | — | — | 9.6 × 10³ |
| 150 | — | — | — | 3.0 × 10¹ |
| Mitomycin B | — | >10^{7b} | NA^{c} | >10^{7d} |

^{a}Did not test strain against this concentration of MC
^{b}Mitomycin B tested at a concentration of 1.0 μg/ml
^{c}Did not test strain against mitomycin B
^{d}Mitomycin B tested at a concentration of 15 μg/ml

Co-expression of mct and mrd in E. coli. To address the notion that MRD and MCT proteins participate as components of a binding protein-dependent drug export system, the mct and mrd genes were co-expressed in E. coli. From plasmid pDHS7006 (mrd expression construct) (Sheldon et al., 1997), a DNA fragment containing the mrd gene under the control of the T7 promoter was ligated into pDHS7023 to give pDHS7024. pDHS7024 was then introduced into E. coli BL21 (DE3) to give strain PJS103. To determine if strain PJS103 was resistant to MC, cultures were grown up and plated on agar medium containing various concentrations of MC. Significantly, IPTG-induced cultures of PJS103 exhibited resistance to MC at drug concentrations 300-fold greater than those for E. coli BL21(DE3) containing vector alone (150 μg/ml vs. 0.5 μg/ml of MC; Table 4). In addition to PJS103 maintaining levels of resistance over that of the vector control strain, co-expression of mct and mrd confers MC resistance at drug concentrations 5 and 60-fold greater compared to PJS100 (containing the mrd gene alone) (Sheldon et al., 1997) or strain PJS102 (containing the mct gene alone), respectively. Strain PJS103 also displayed high-level resistance to mitomycin B (Table 4), a mitomycin also produced by S. lavendulae.

MC uptake by E. coli cells expressing mct, mrd or mct/mrd. Since the deduced amino acid sequence of the mct gene was similar to antibiotic export proteins, reduced accumulation of MC in MCT-expressing cells would be expected. An assay, modeled after experiments used to study tetracycline efflux-mediated resistance in E. coli (Levy and McMurry, 1978), was designed to study the uptake of [³H]-MC by the susceptible vector control and resistant mct, mrd and mct/mrd expressing E. coli strains.

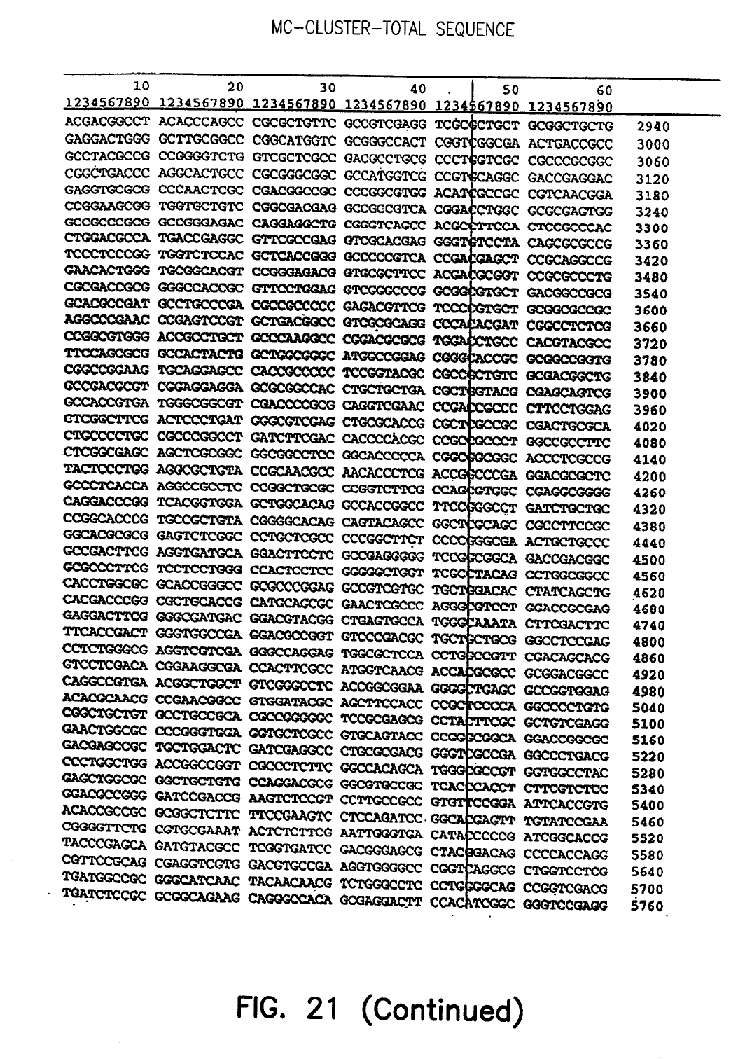

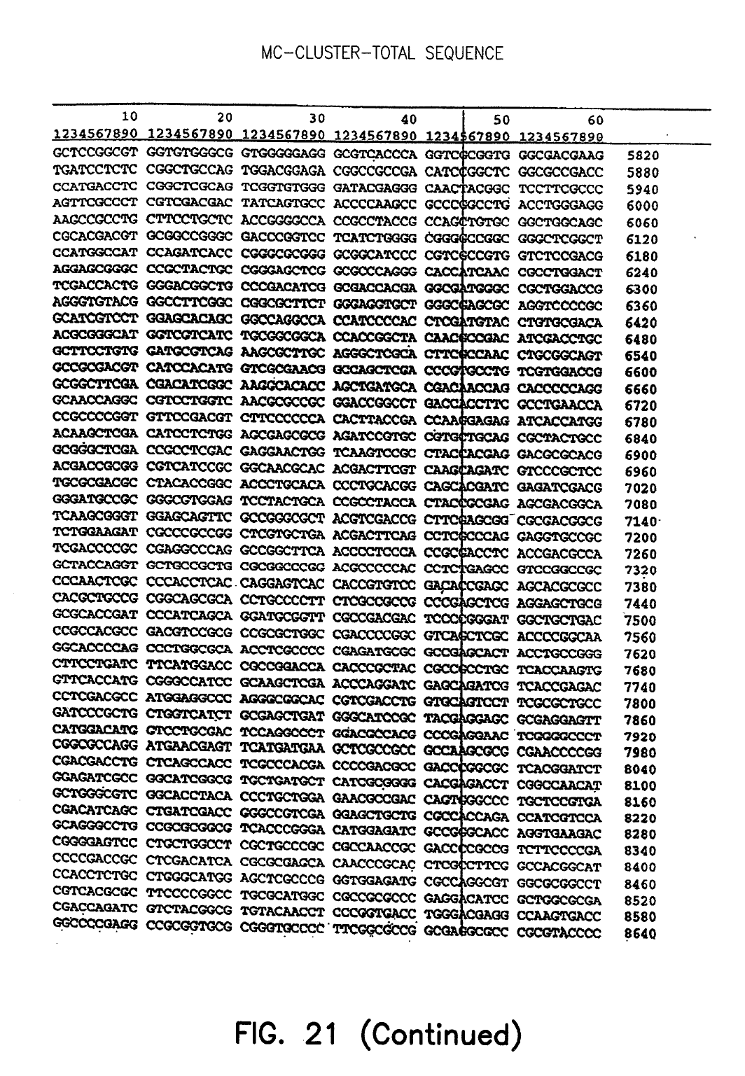

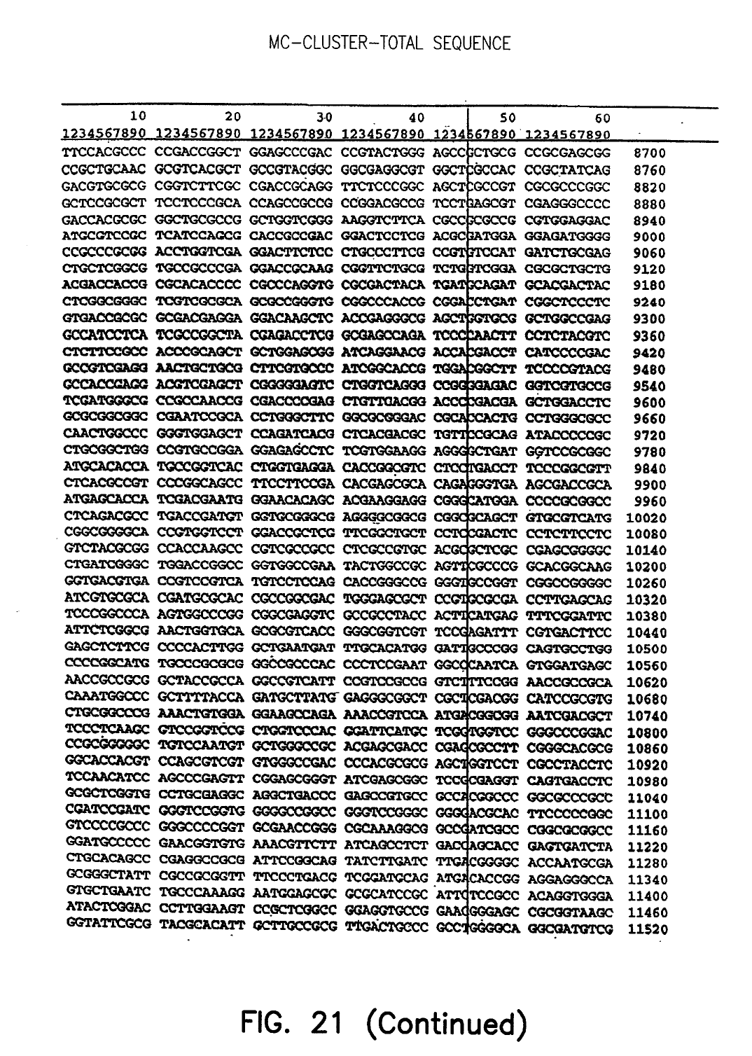

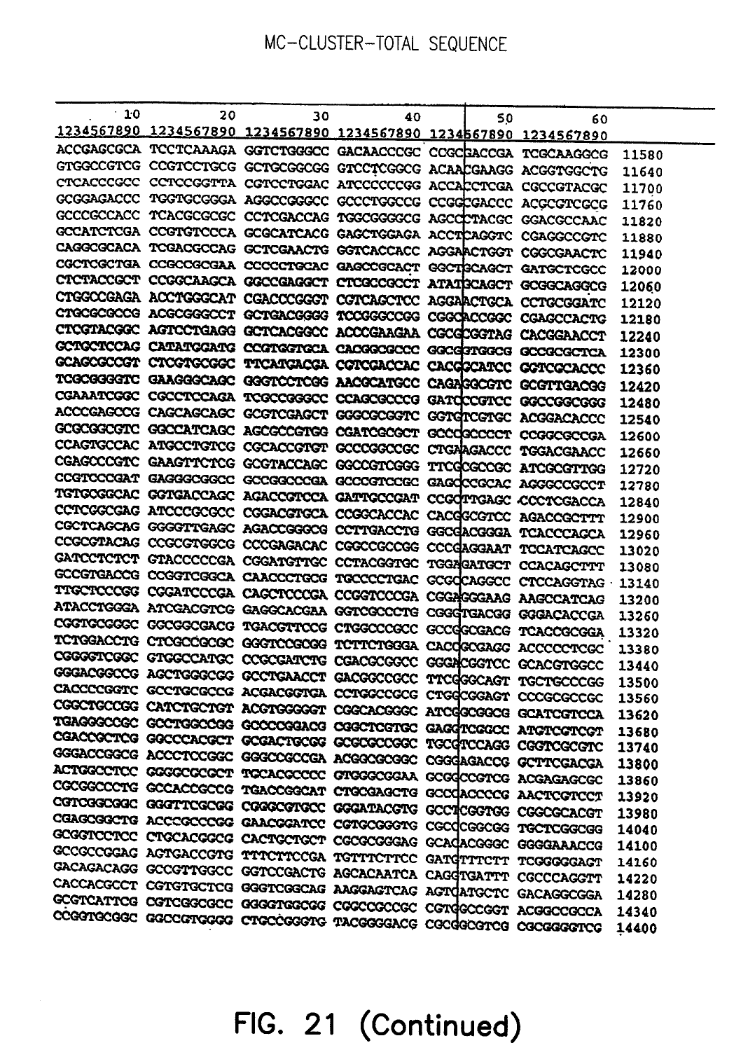

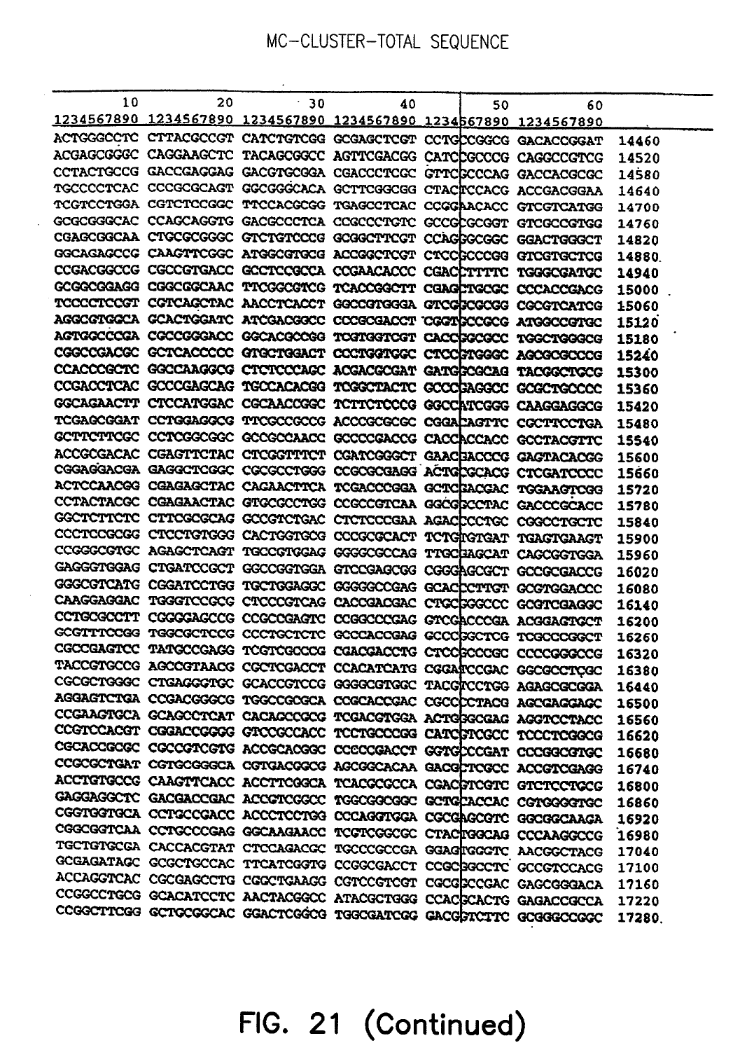

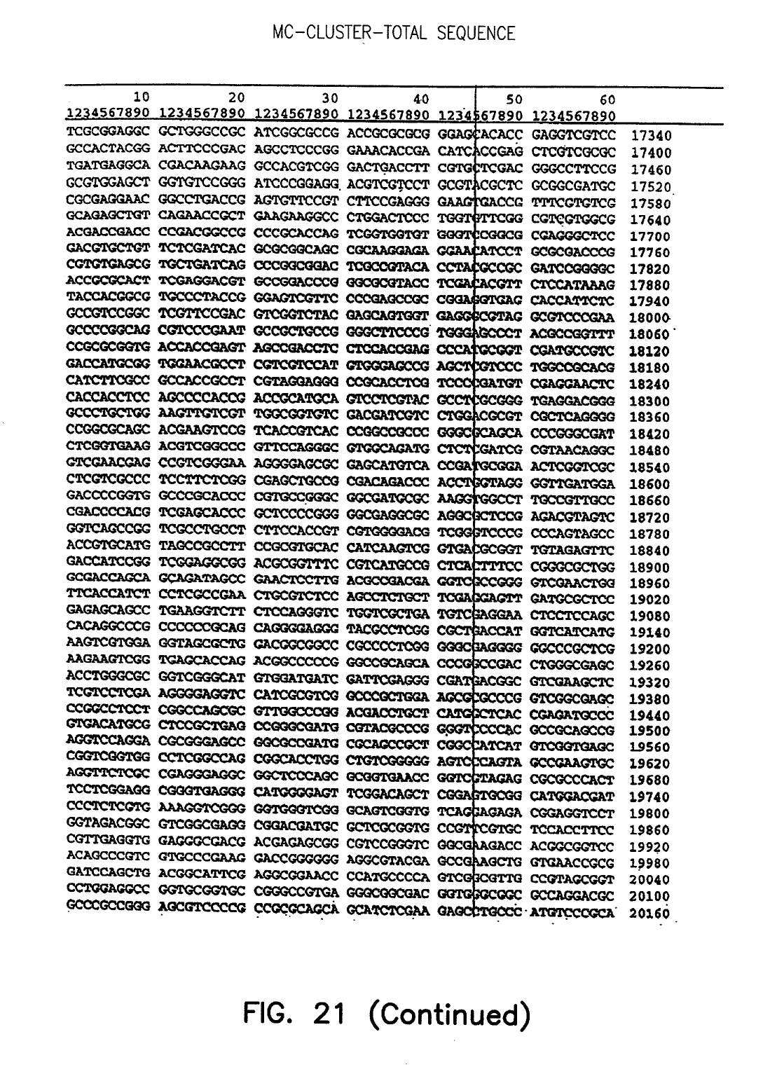

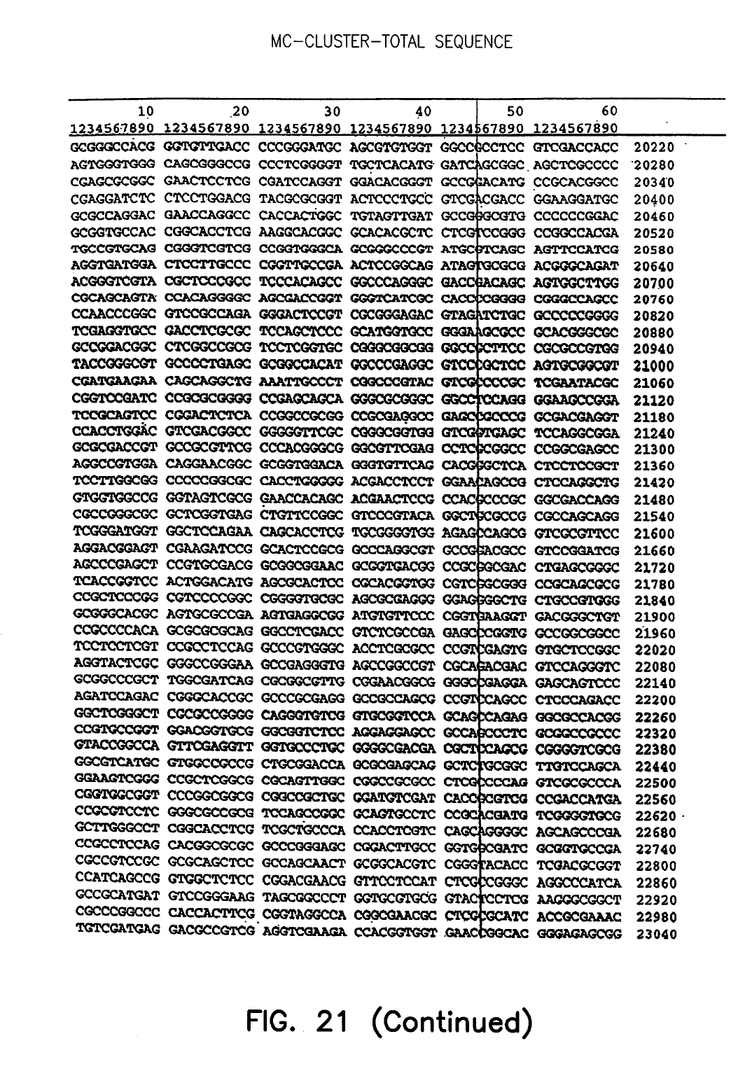

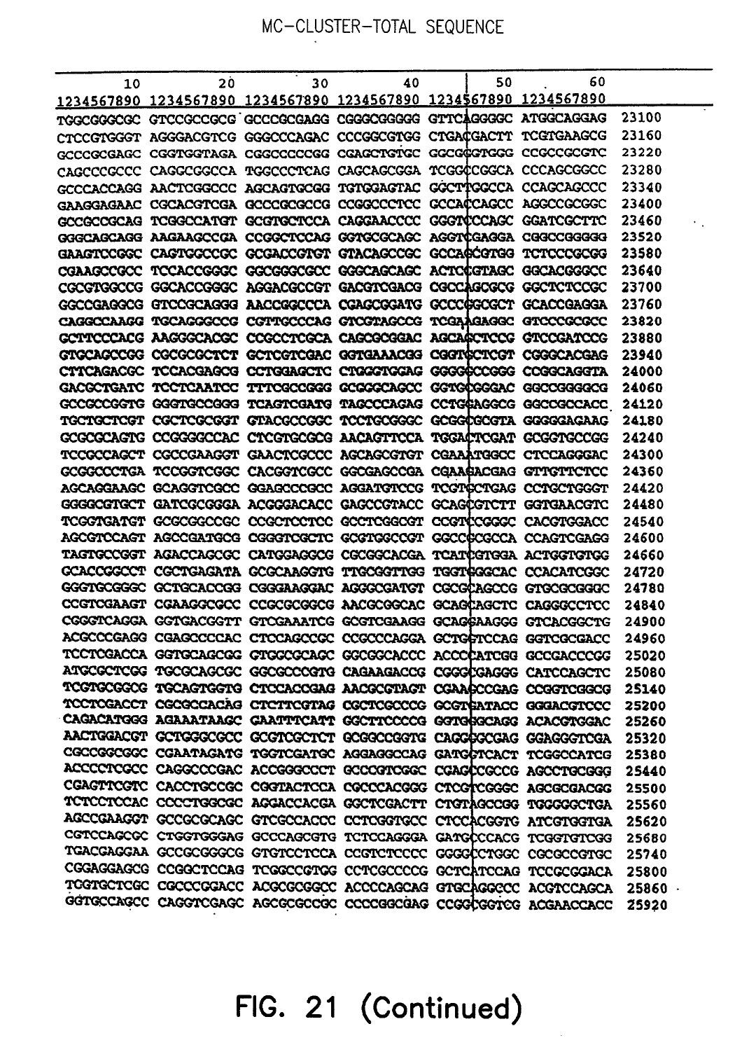

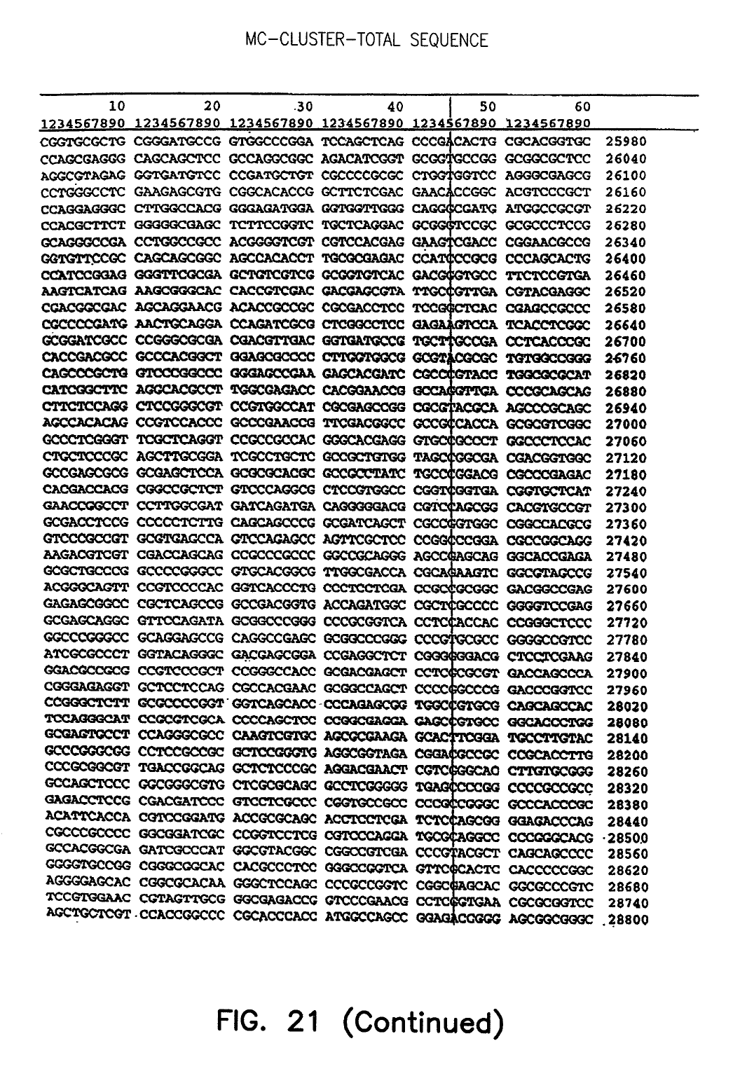

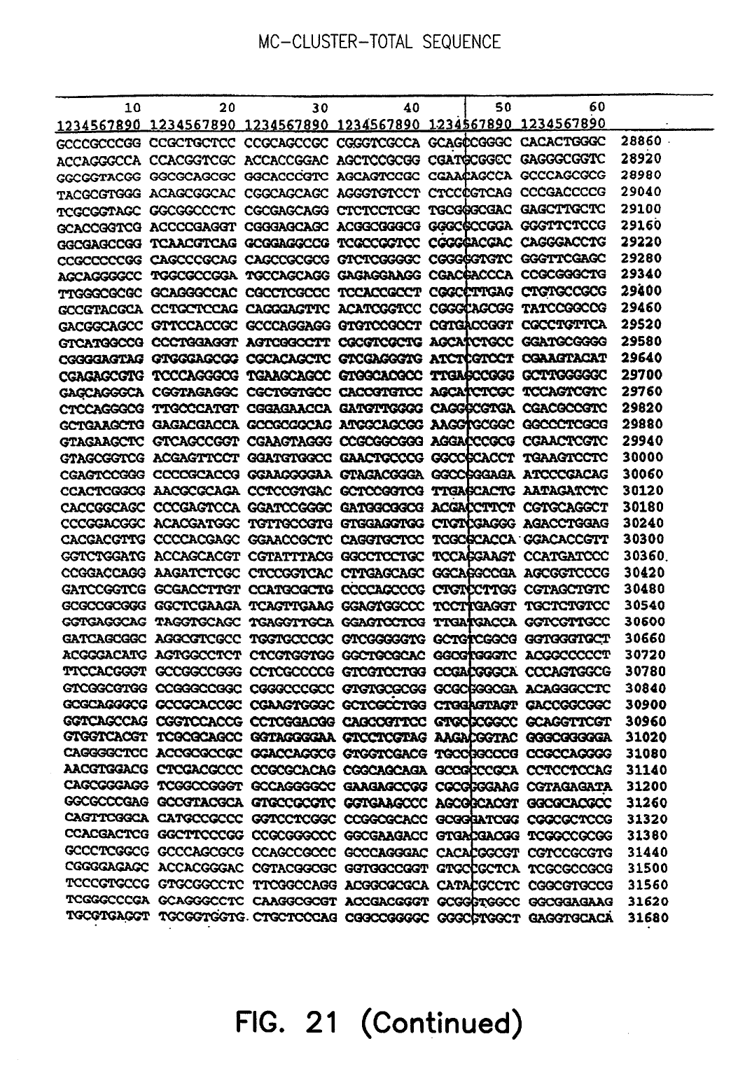

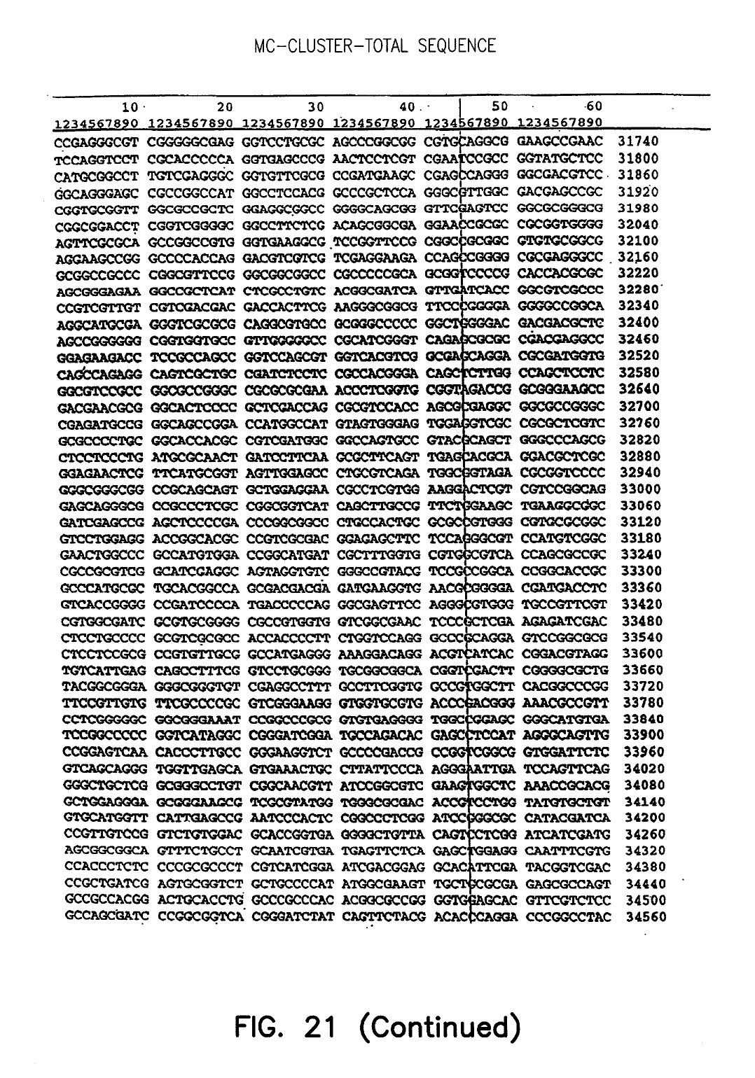

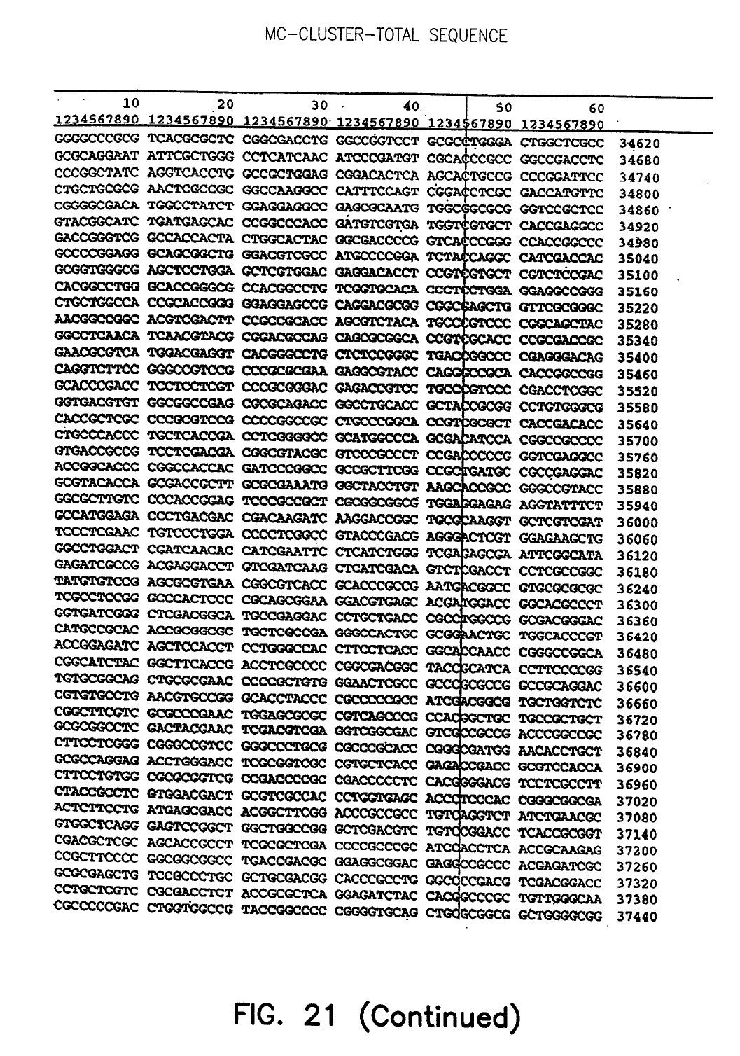

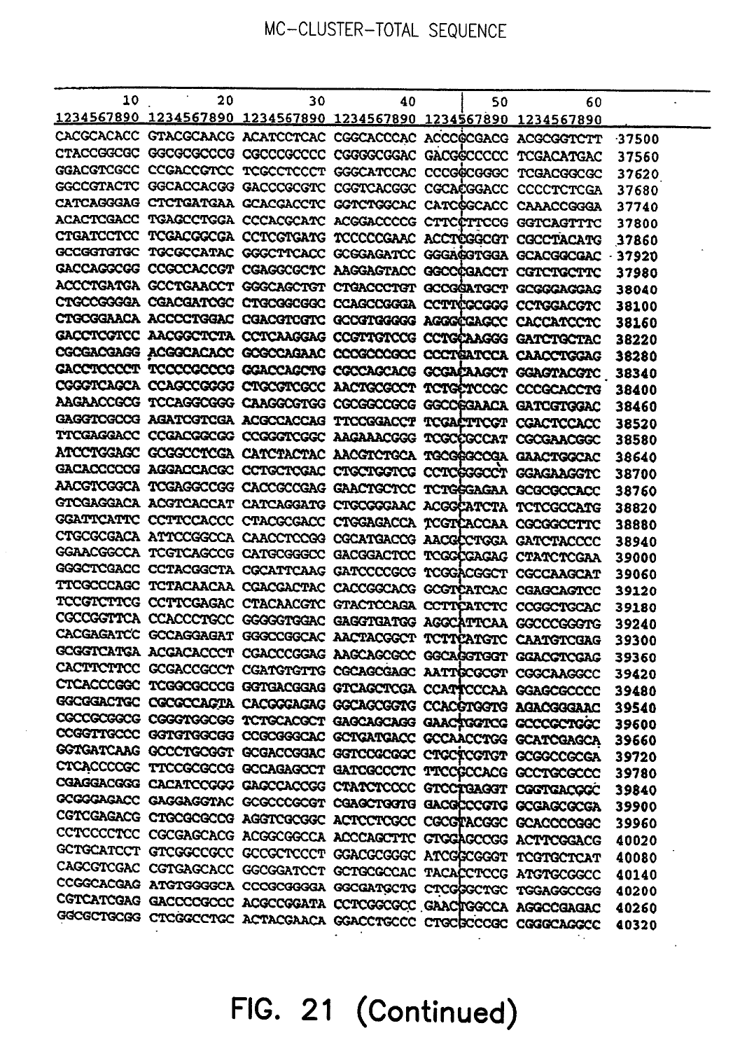

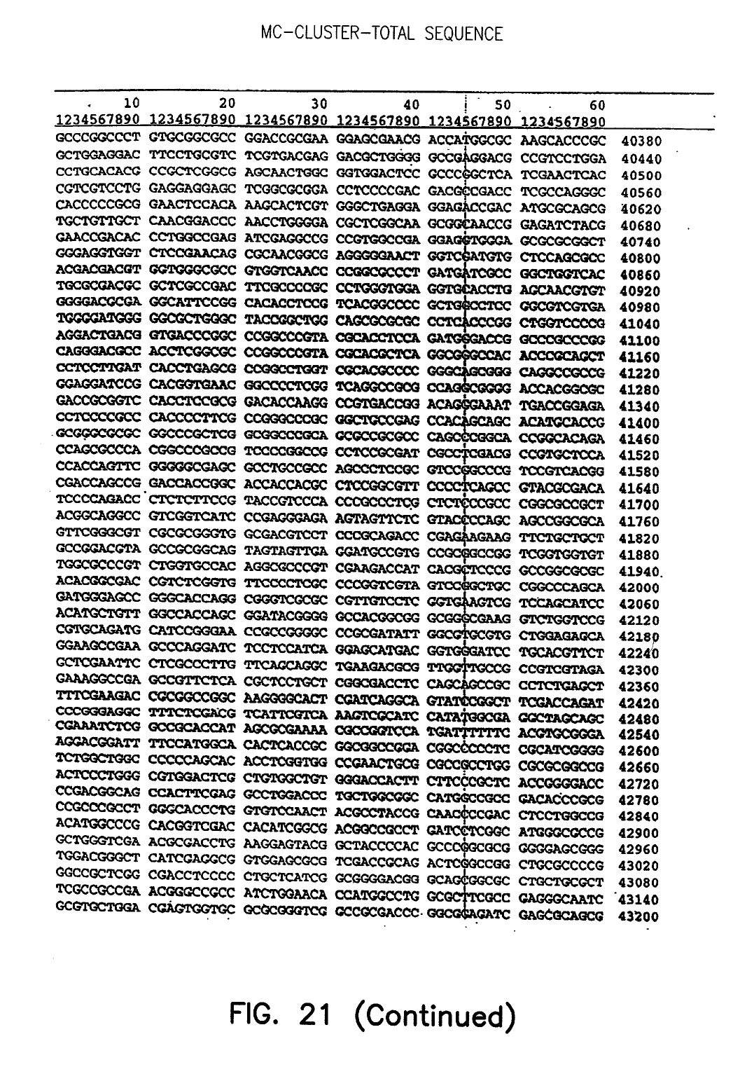

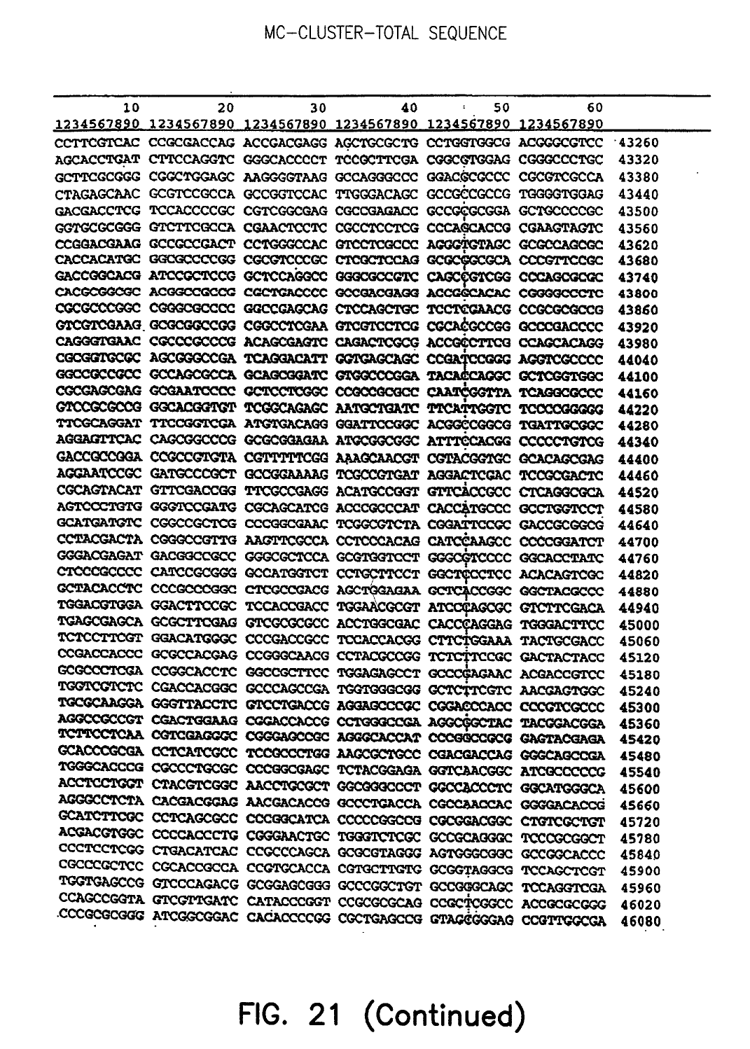

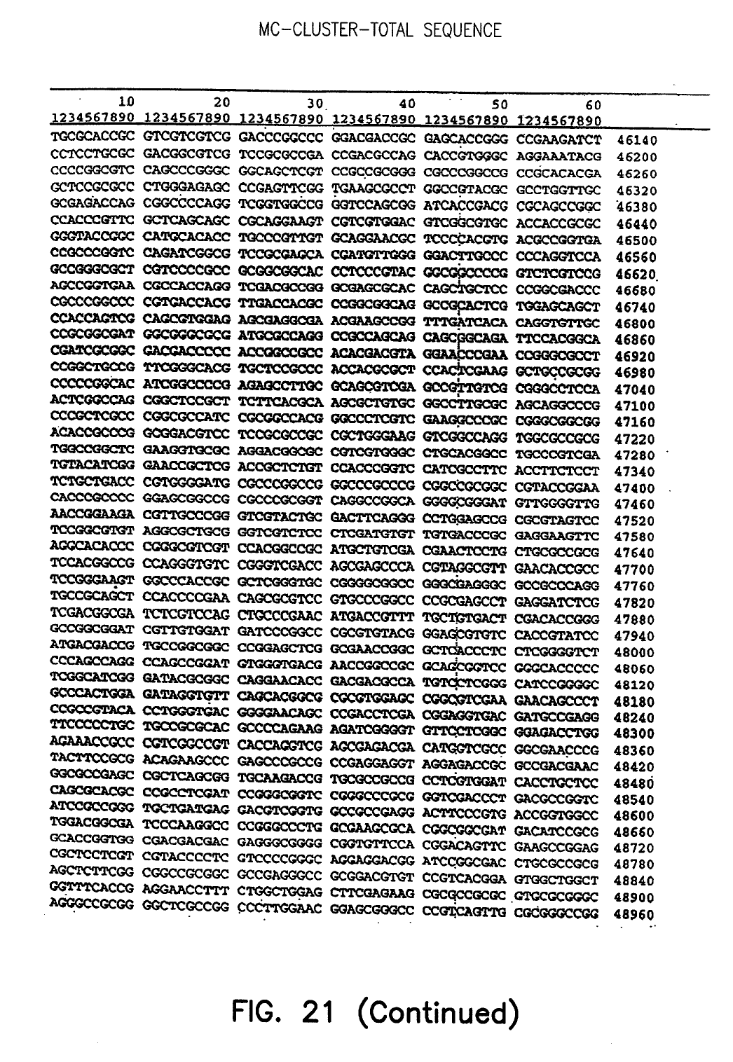

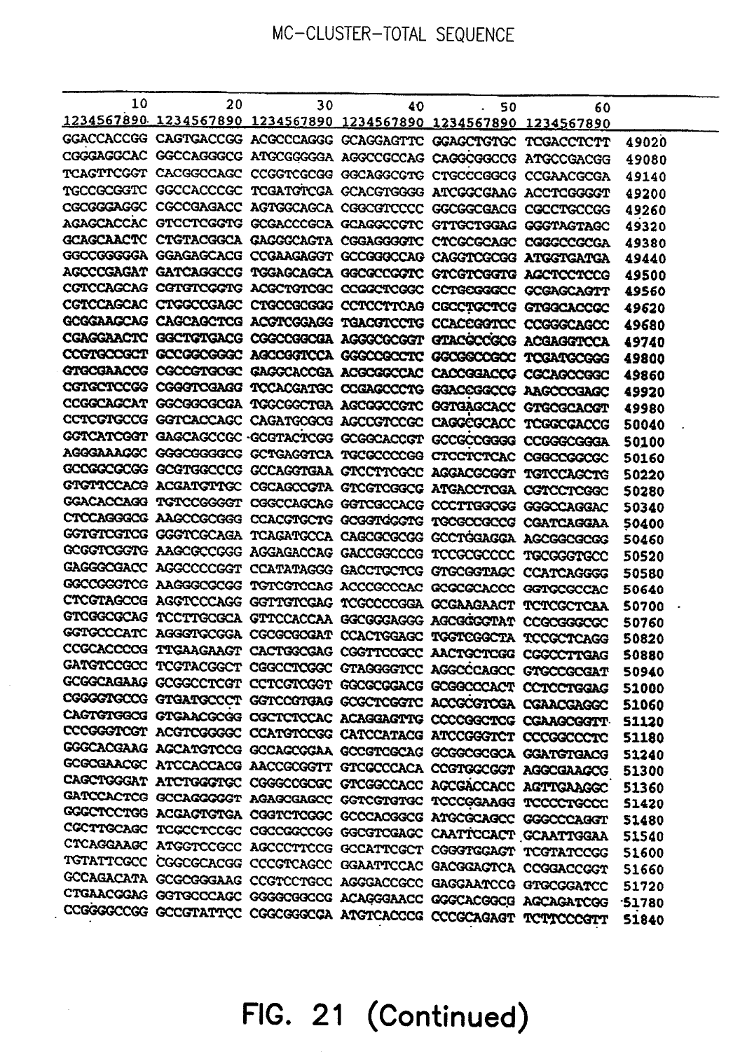

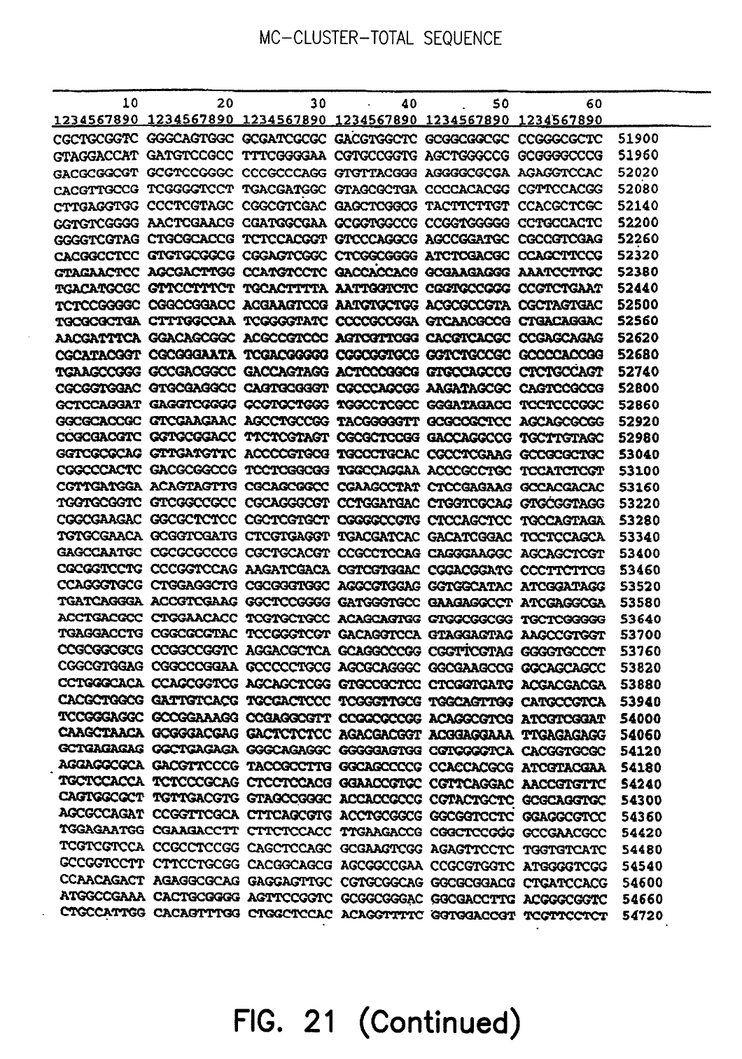

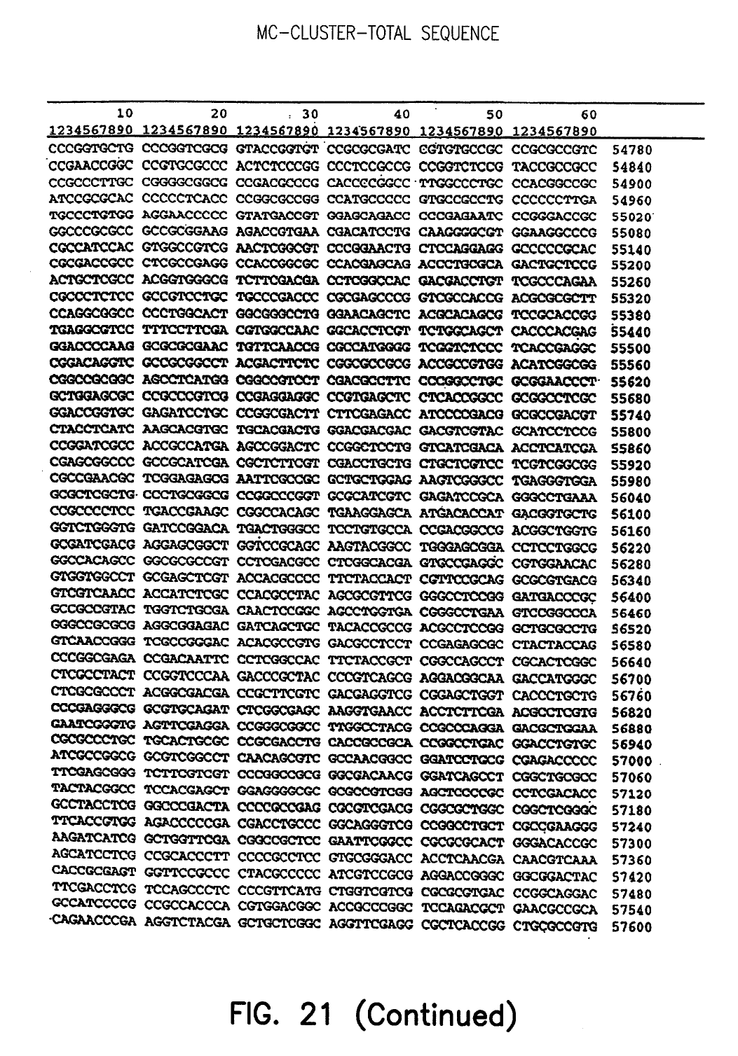

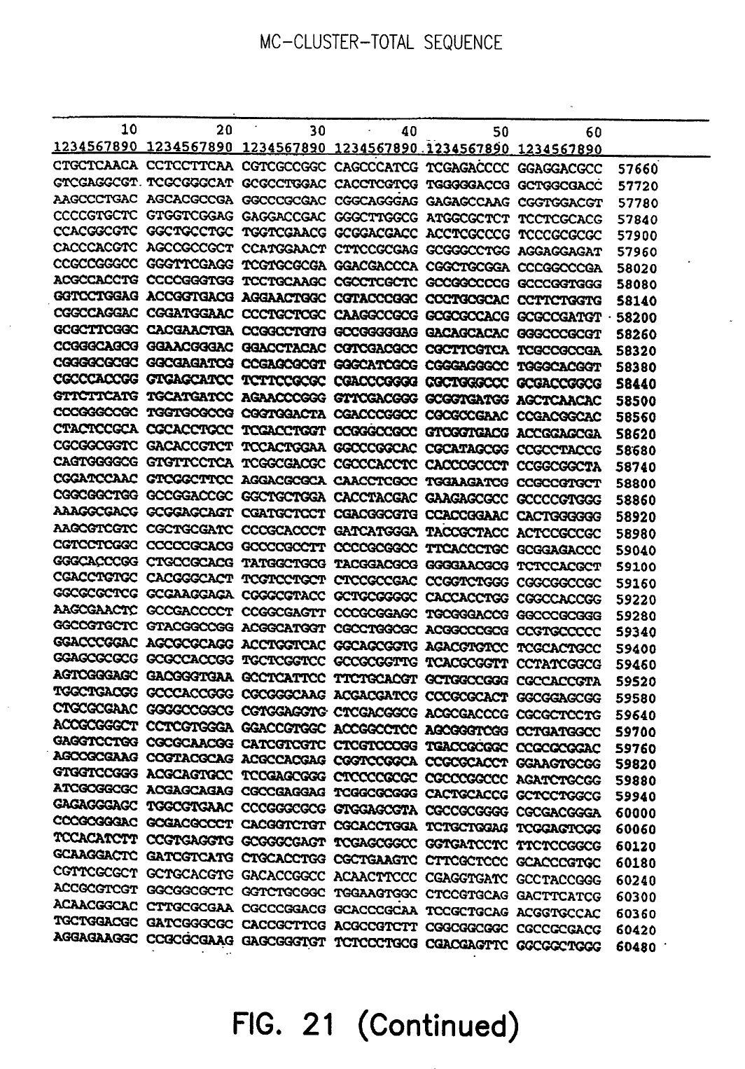

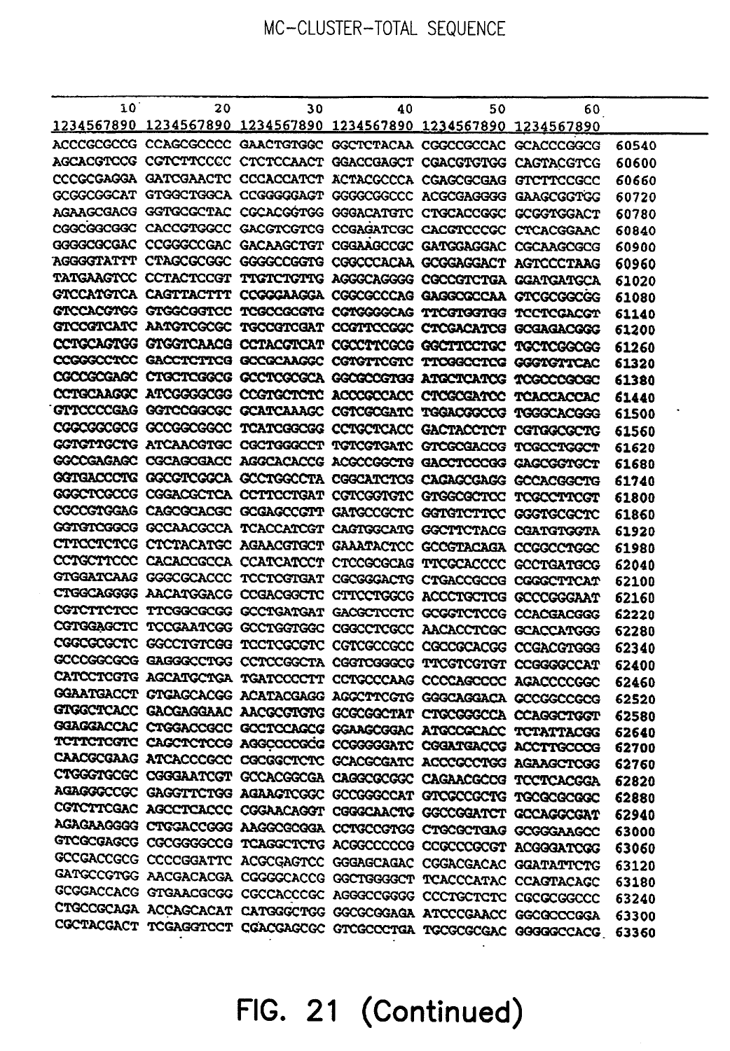

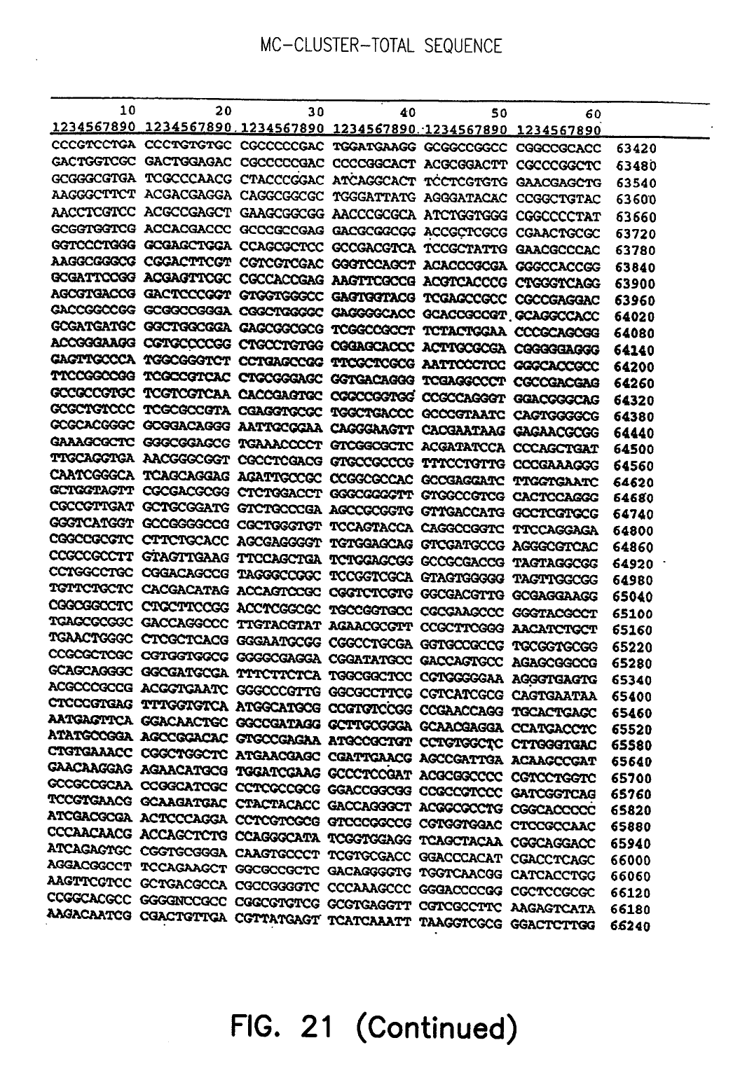

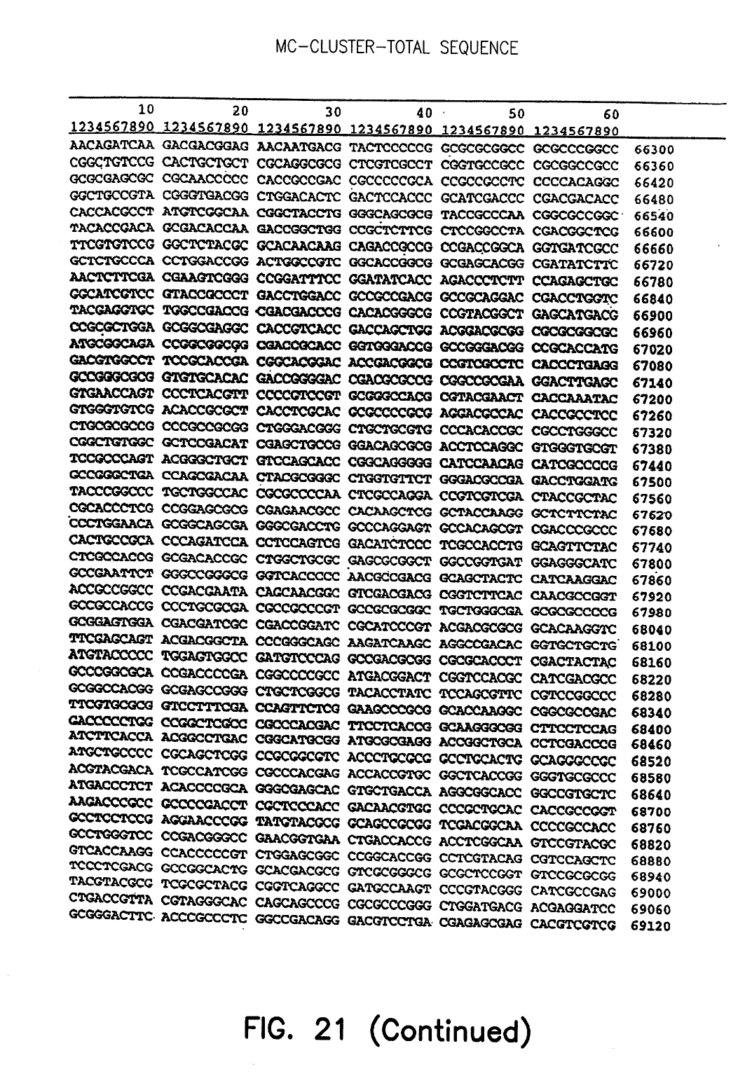

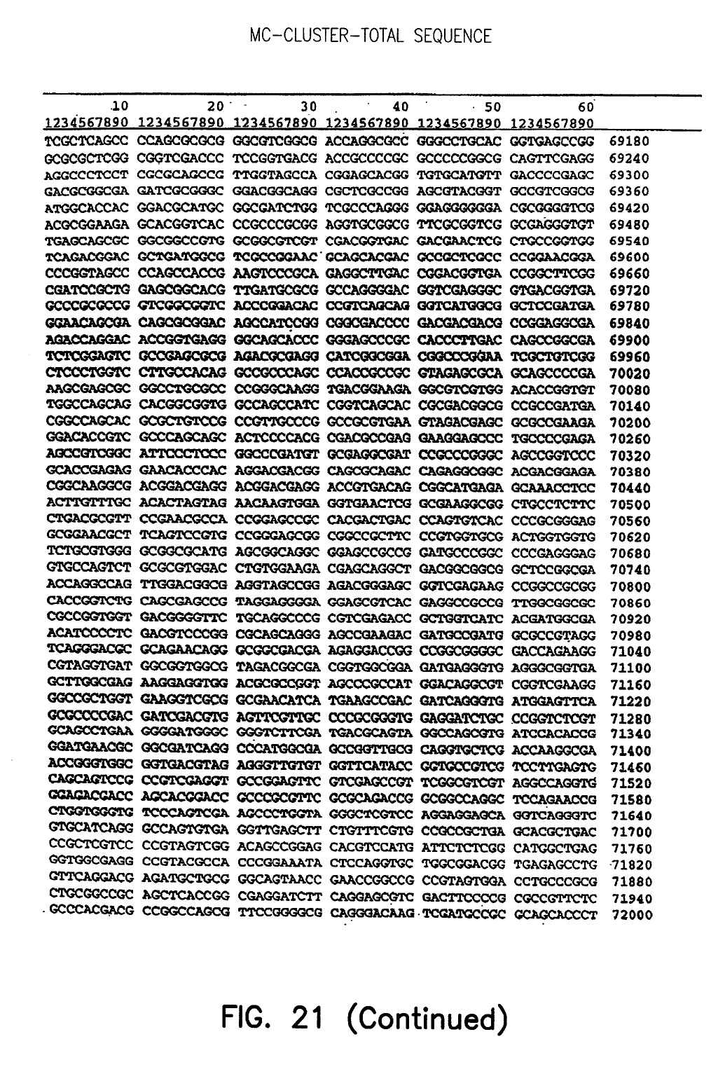

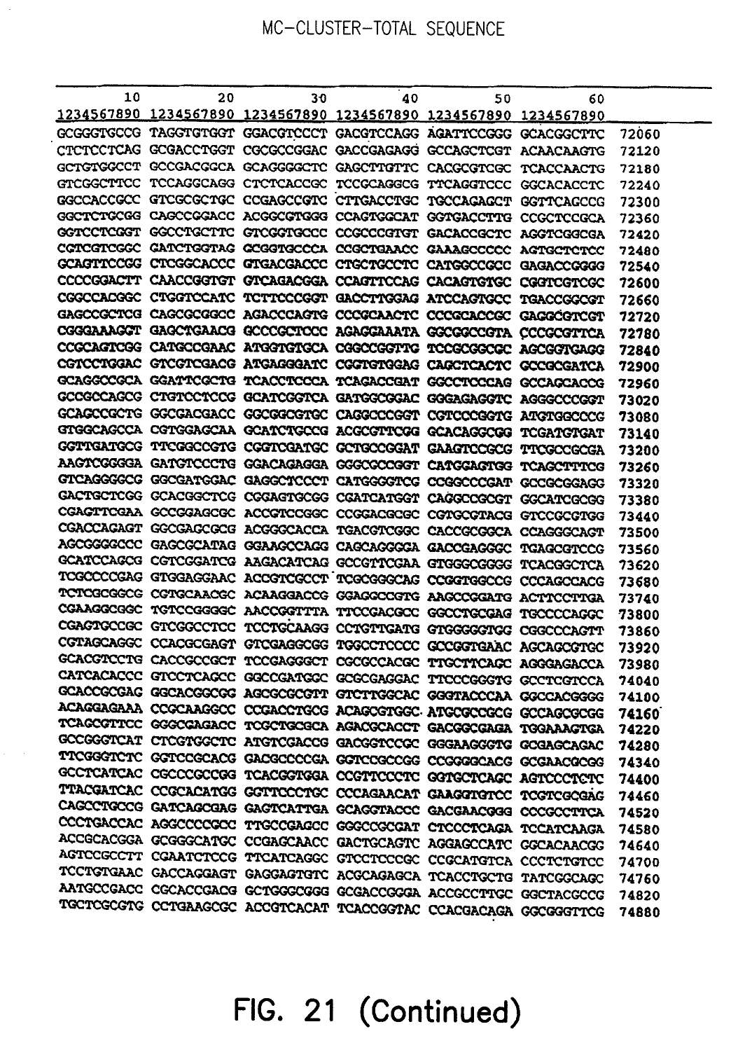

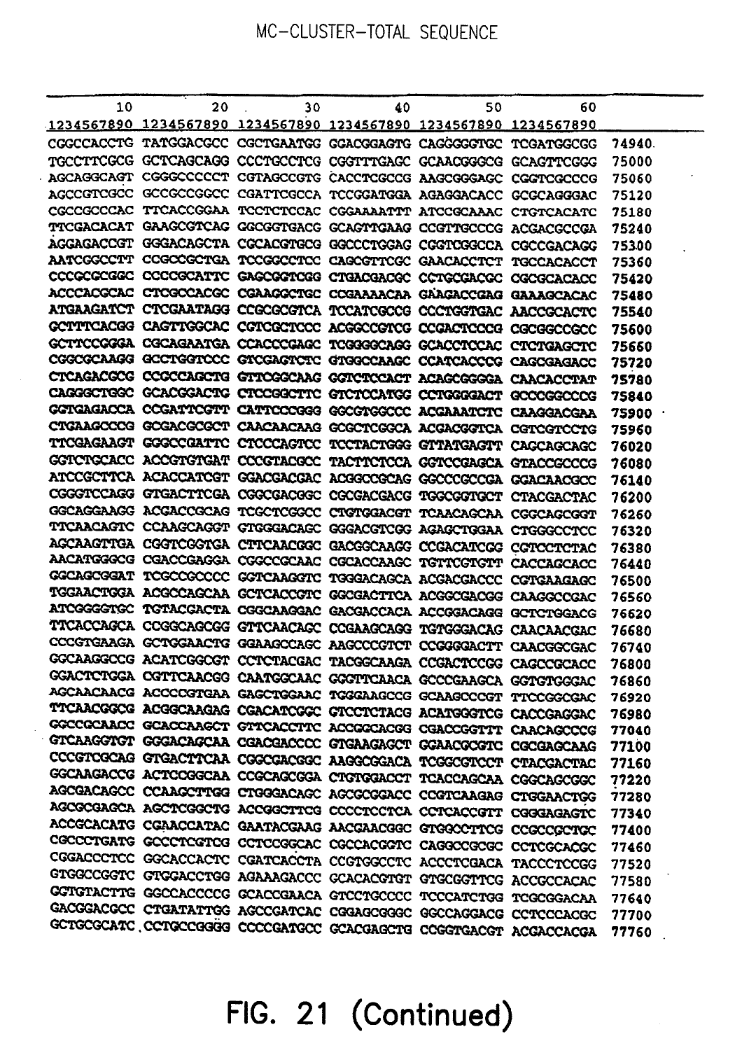

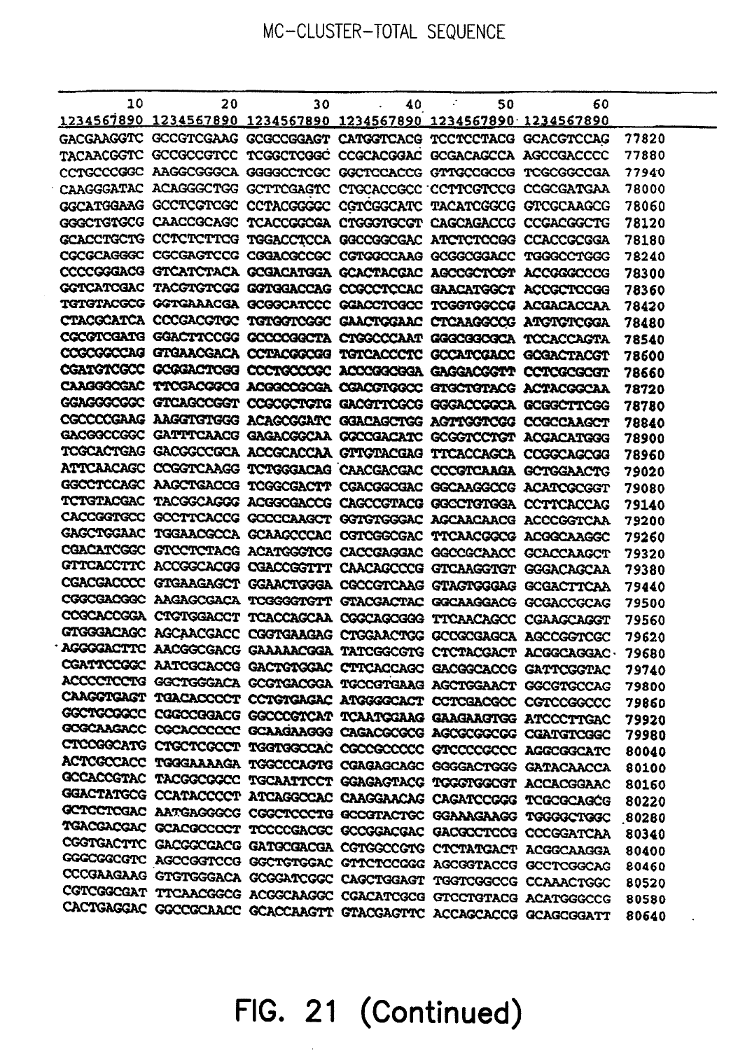

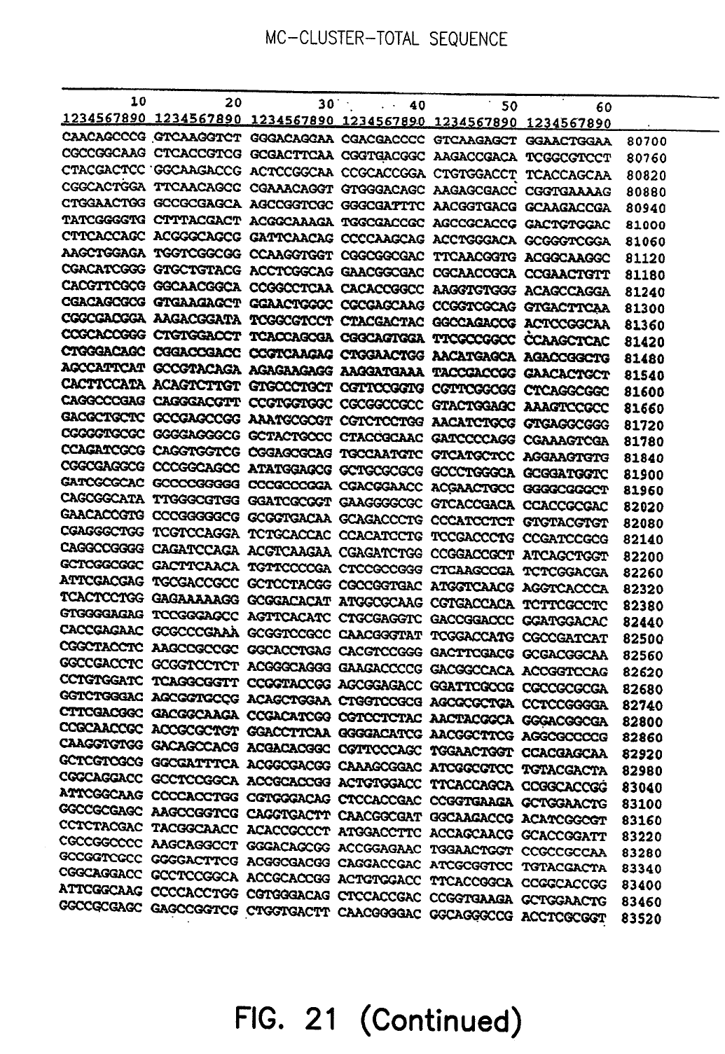

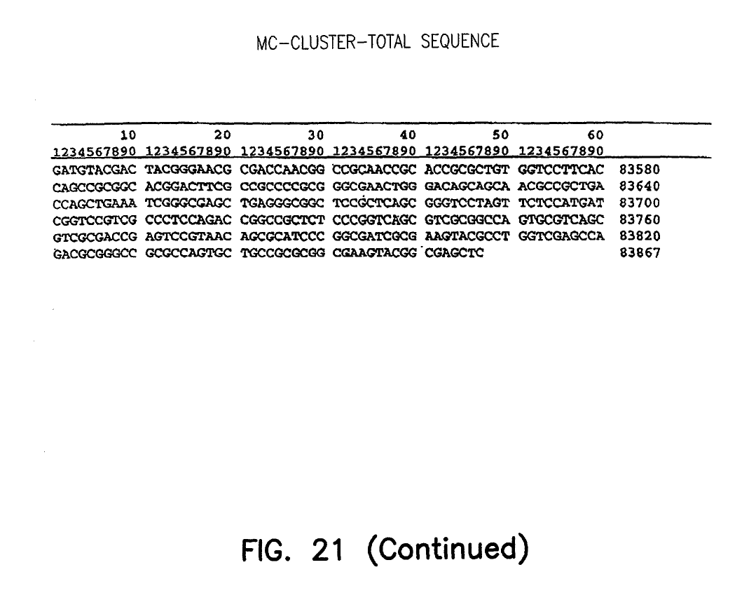

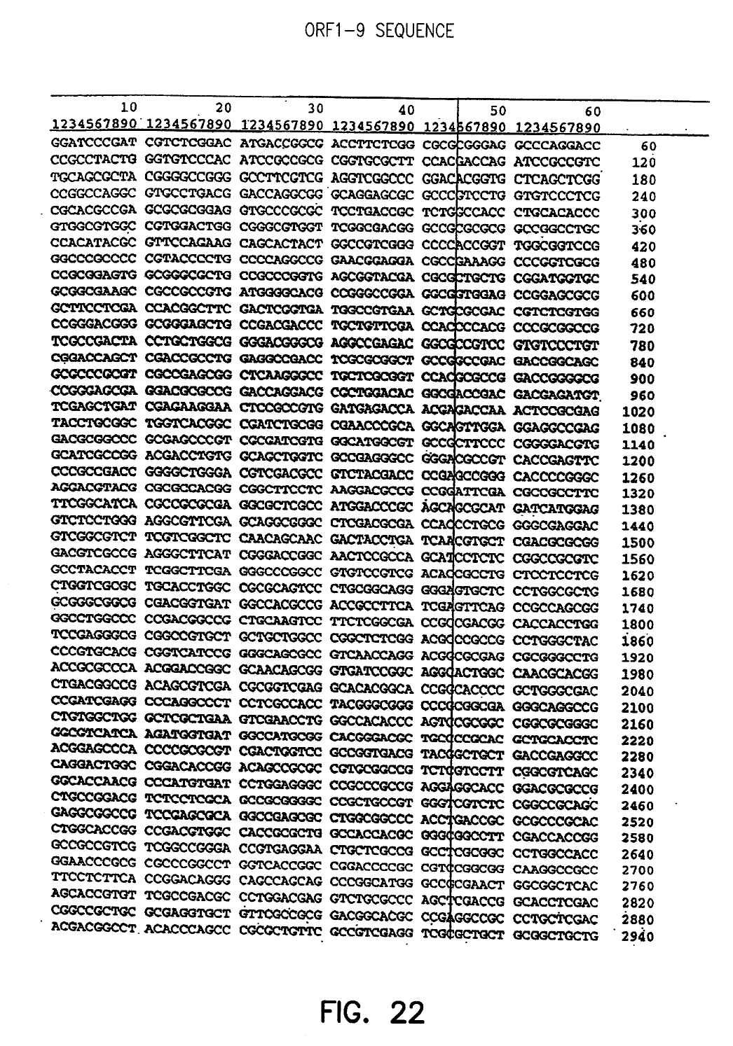

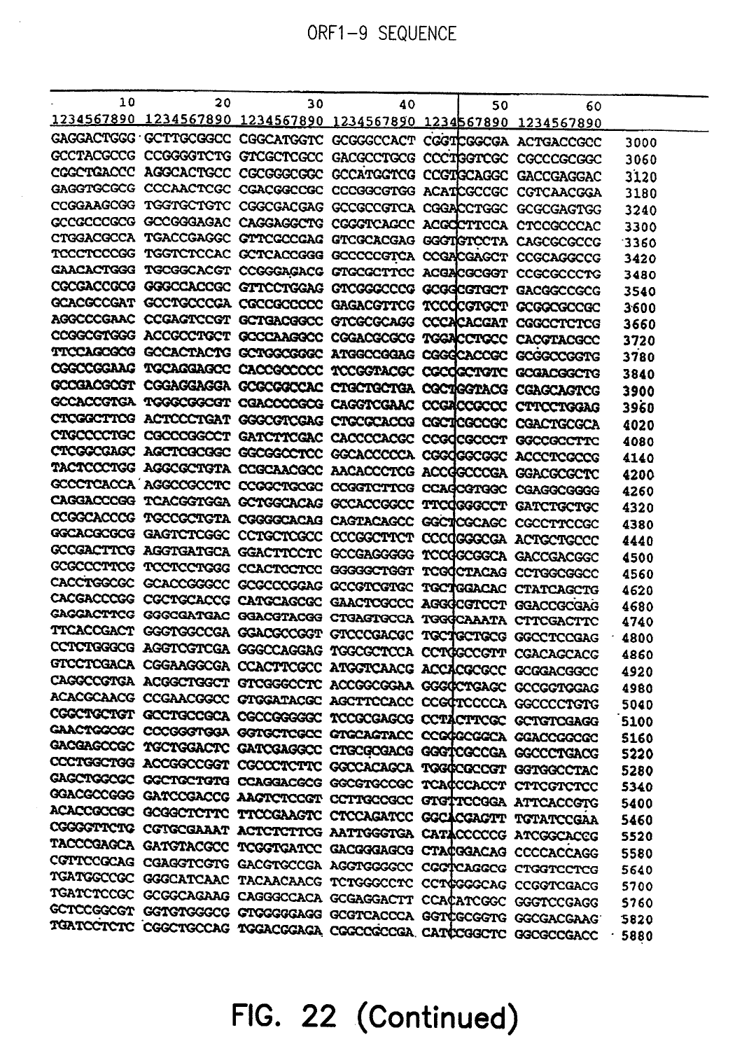

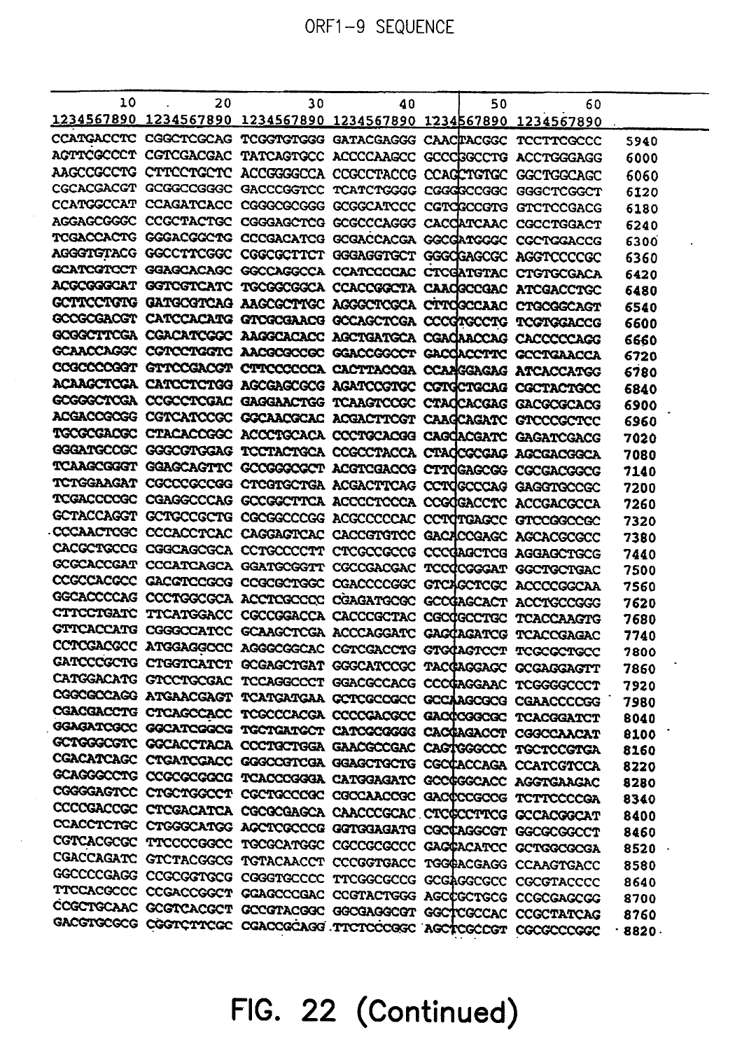

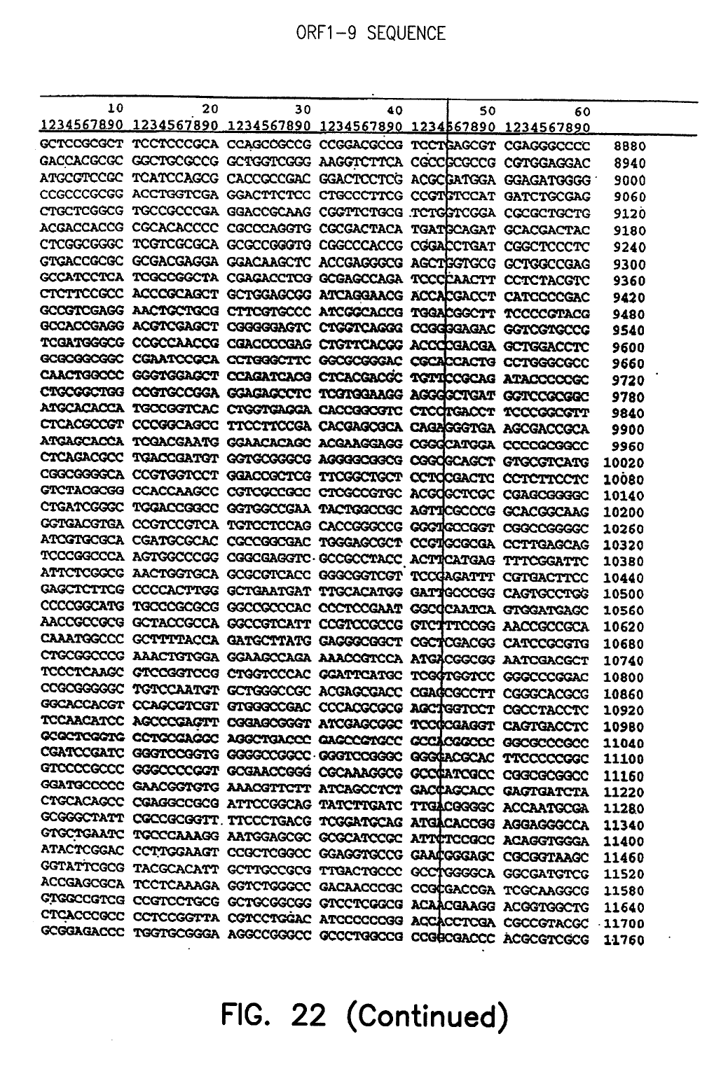

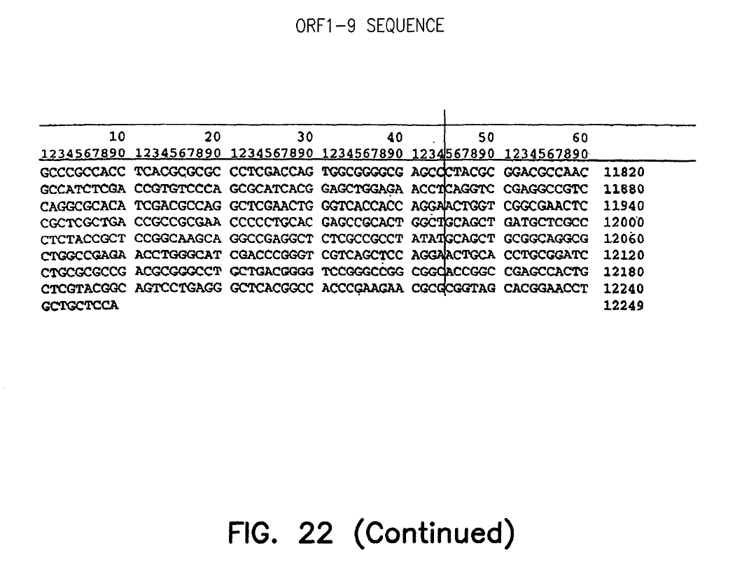

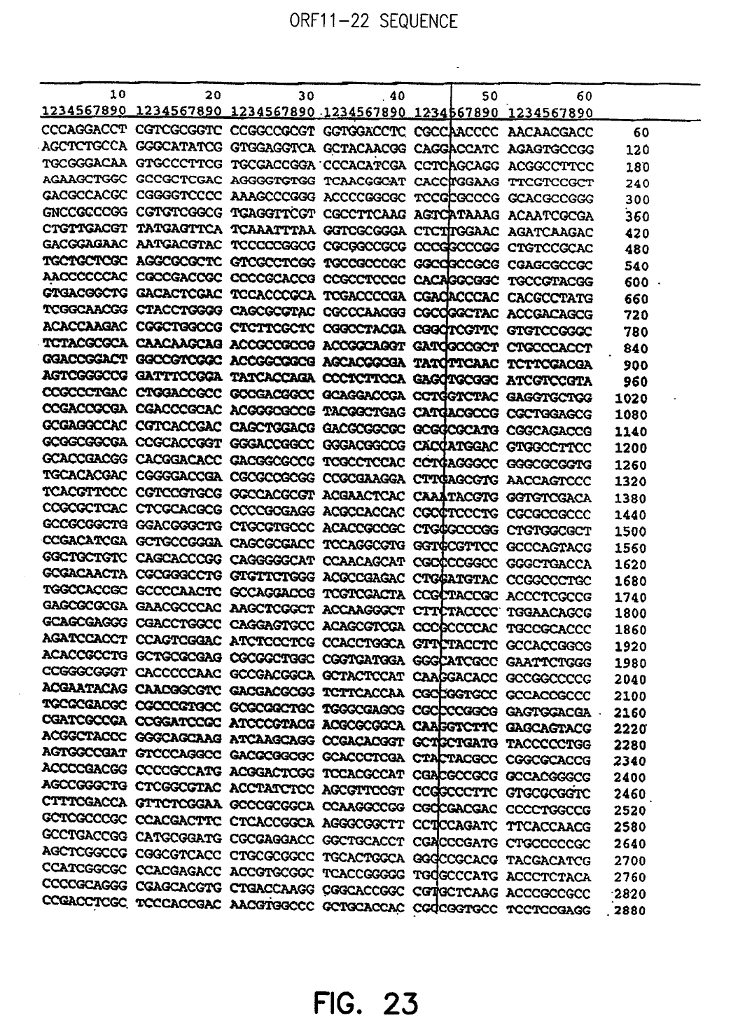

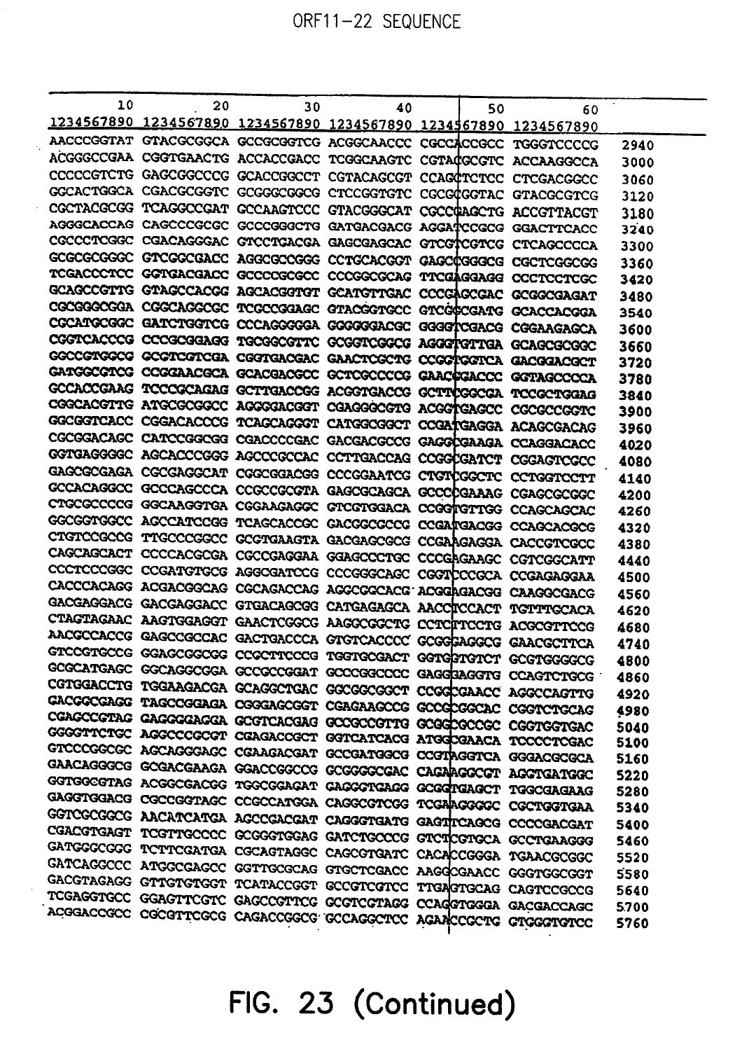

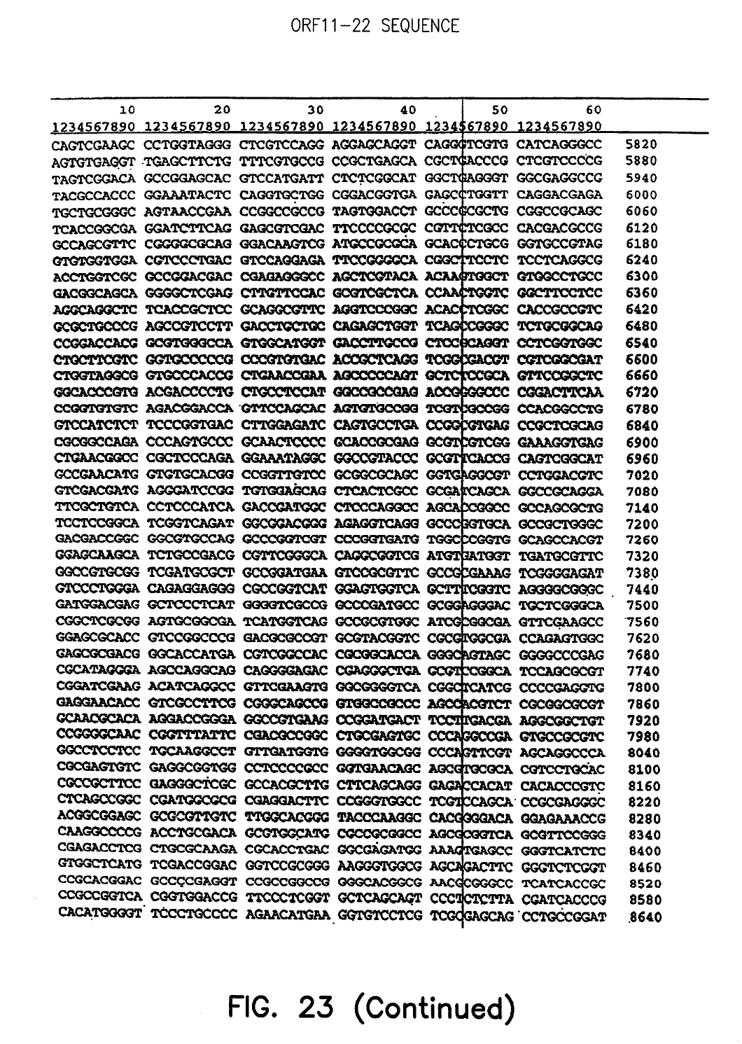

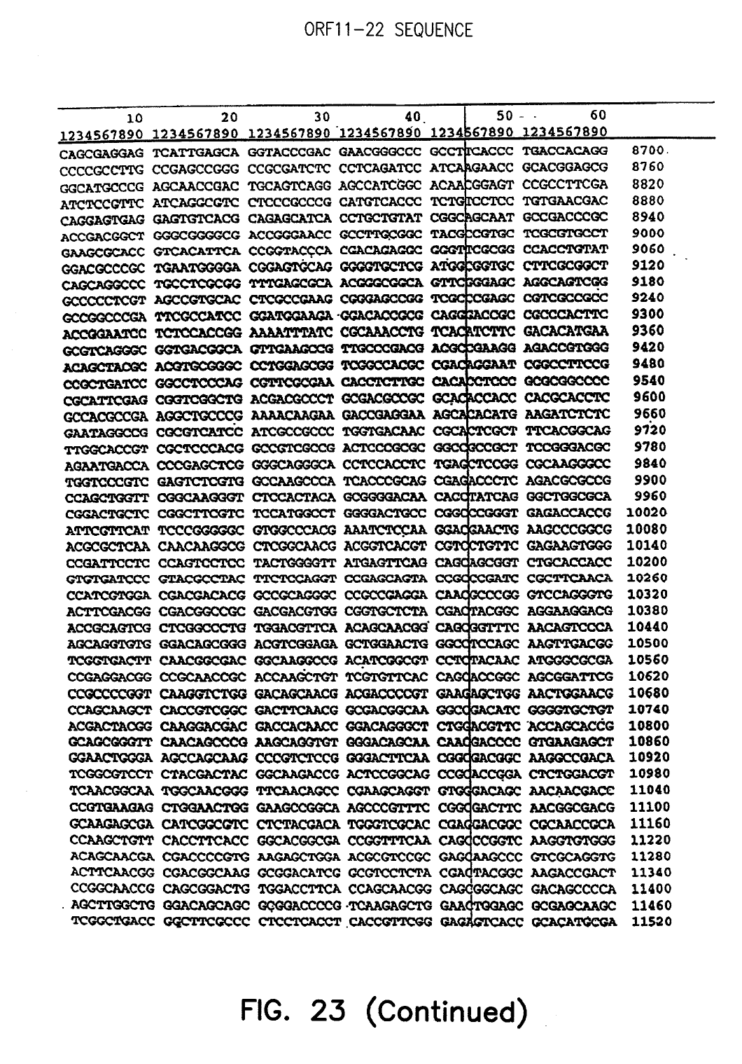

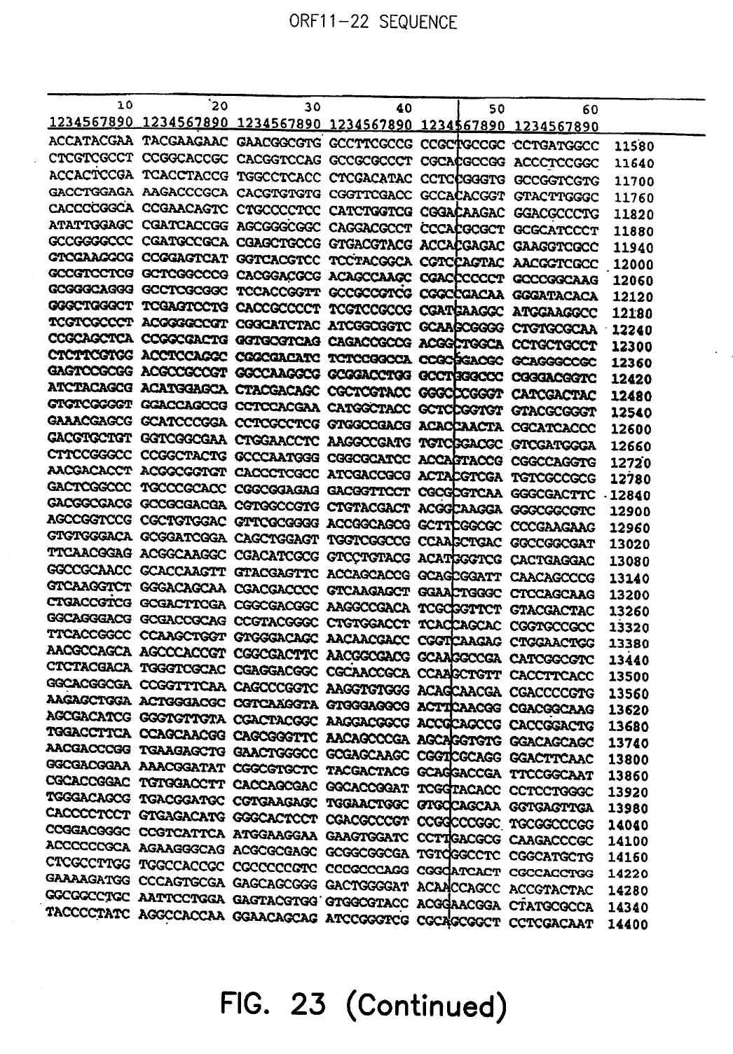

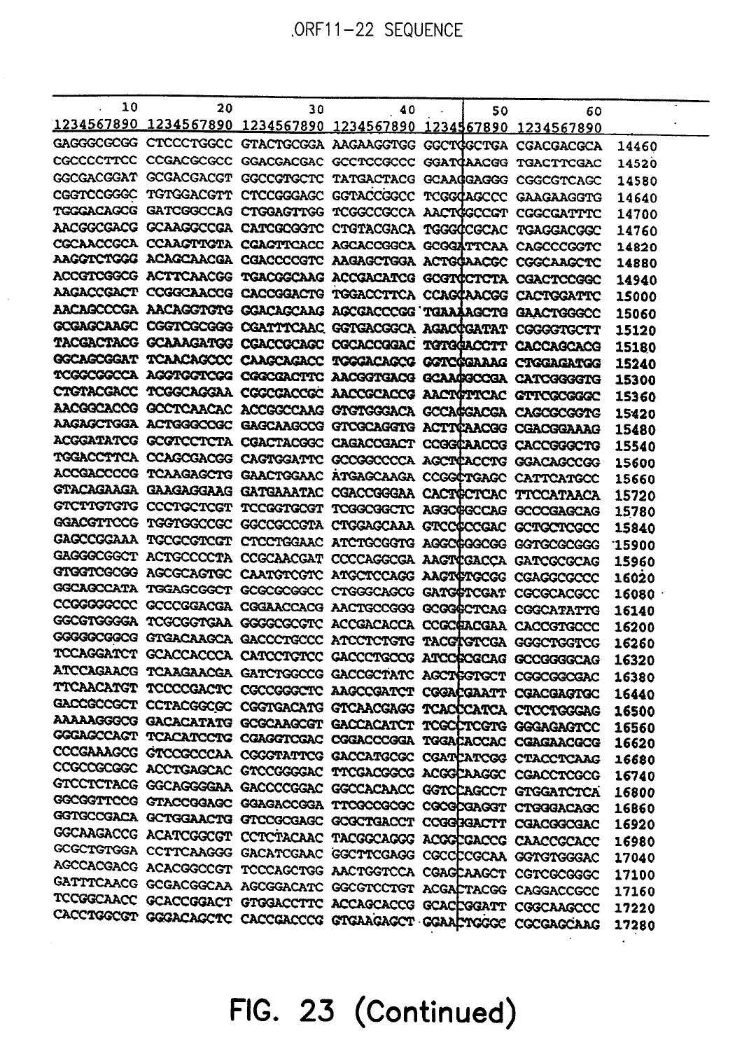

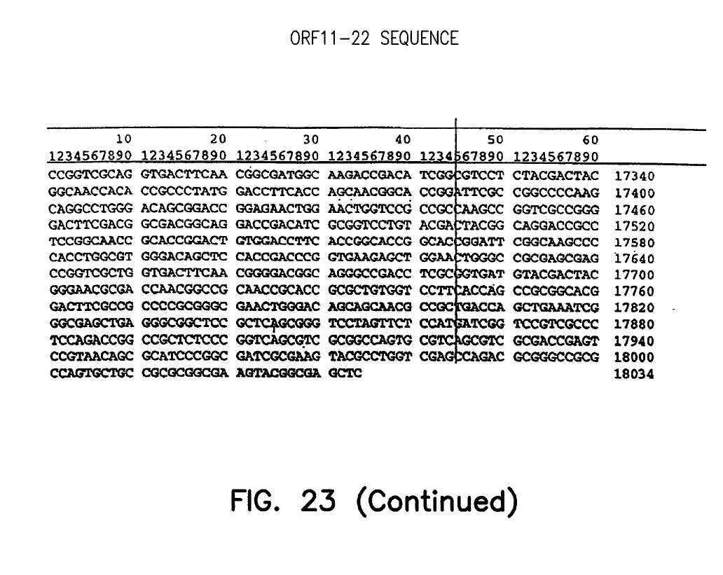

MC accumulation by the susceptible vector control strain (BL21(DE3)::pET17b) was found to reach a maximum level at 5 minutes and thereafter maintained at constant concentrations. In contrast, the quantity of MC accumulation in the resistant, mct-expressing strain (PJS102) was only 25% of the susceptible control at 5 minutes, and thereafter remained at reduced concentrations (FIG. 21). Reduced accumulation of drug in PJS102 suggests that mct encodes a protein that facilitates MC export from the cell. To determine if the co-expression of mct and mrd in E. coli also resulted in reduced accumulation of MC, strain PJS103 was analyzed using the [3H]-MC uptake assay. Analyses of drug uptake by cultures of strain PJS100 (Sheldon et al., 1997) were also performed to determine drug accumulation levels in this MC resistant E. coli strain.

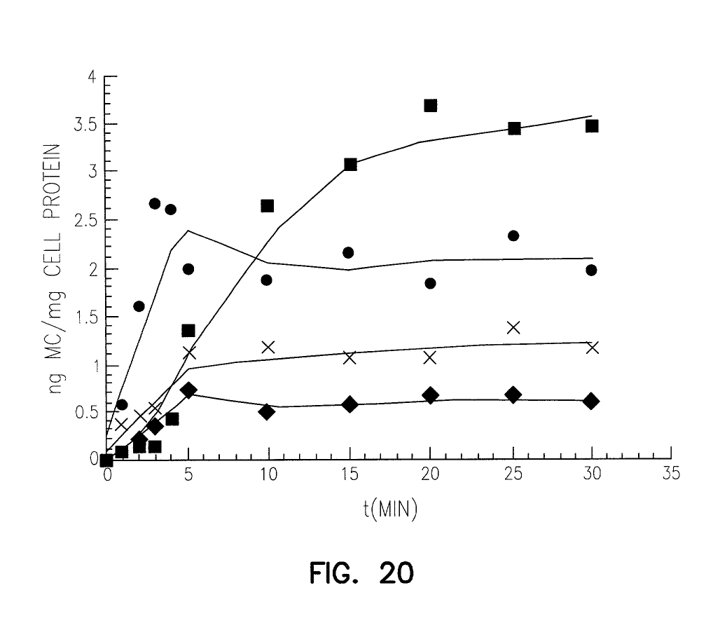

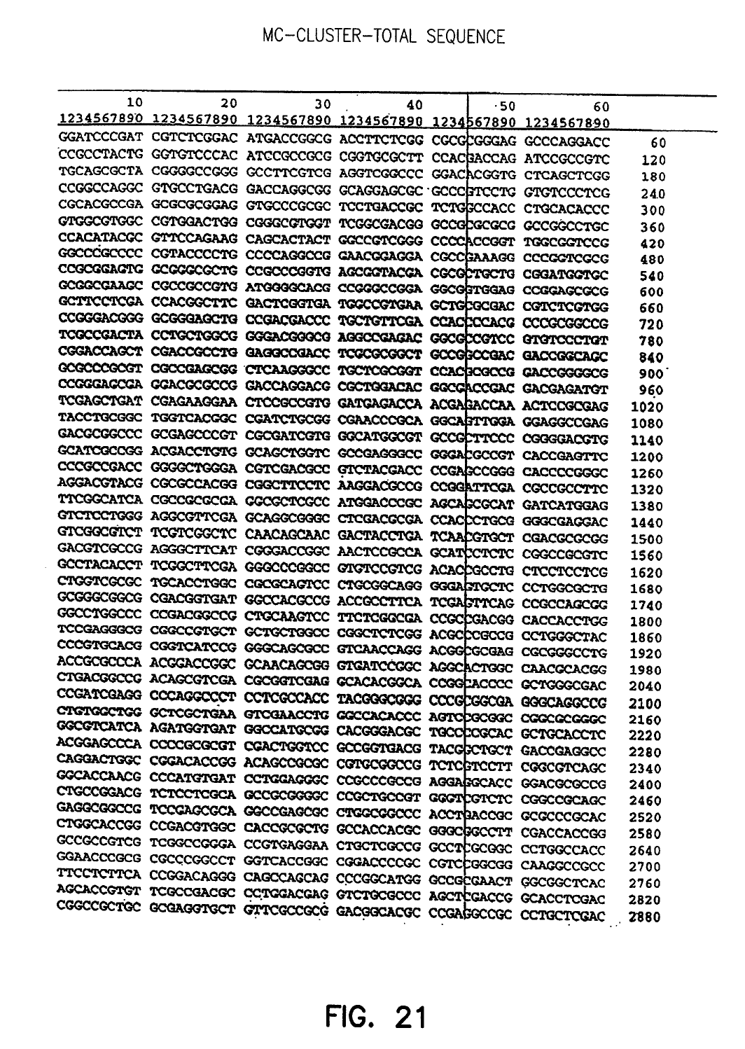

The results show a clear difference in MC accumulation between the MC sensitive and resistant strains. Compared to E. coli cells bearing vector alone, MC accumulation in PJS103 was only 35% at 5 minutes and thereafter remained at reduced concentrations. The accumulation of drug in strain PJS103 was found to parallel that of strain PJS102, albeit at slightly higher levels (about 23% greater) of drug over the course of the experiment. Interestingly, strain PJS100, although resistant to significant concentrations of MC, accumulated drug to levels 42% higher than the drug-sensitive vector control at 30 minutes (FIG. 20).

Discussion

Most antibiotics inhibit bacterial growth by binding to proteins or other macromolecular components that involve essential metabolic processes of the cell (Cundliffe, 1992). For instance, DNA alkylation by MC results in disruption of chromosomal replication leading to cell death (Iyer and Szybalski, 1964). In many antibiotic-producing streptomycetes, macromolecular target site(s) are likewise susceptible to endogenous cytotoxic compounds (that is certainly the case in S. lavendulae). Thus, pumping the antibiotic out of the cell at a rate equal to its production and/or re-uptake would prevent drug access to intracellular target sites. Based on the levels of drug found in most antibiotic fermentation broths (concentrations of intracellular drug being low), it is apparent that drug-producing organisms often depend on efficient antibiotic transport mechanisms. Indeed, a growing number of membrane systems implicated in transport (and therefore resistance) of a variety of antibiotics have been discovered in drug-producing streptomycetes (Mendez and Salas, 1998; Paulsen et al., 1996).

In general, genes coding for drug export proteins are physically linked to the corresponding biosynthetic genes within the genome of the antibiotic-producing microorganism. Presumably, the tight linkage of antibiotic export and biosynthetic genes ensures coordinate gene regulation. Interestingly, the presence of back-to-back and overlapping divergent promoters of antibiotic export and regulatory genes has been observed within the tetracenomycin (Guilfoile and Hutchinson, 1992) and actinorhodin (Caballero et al, 1991) biosynthetic gene clusters. Conforming to this example, S. lavendulae possesses a gene coding for an integral membrane drug export protein within the mitomycin biosynthetic gene cluster. Analysis of the deduced amino acid sequence of MCT revealed several similarities with actinomycete proteins predicted to function as drug exporters. By virtue of homology to tetracycline resistance proteins, which have been shown to use proton motive force to energize transport (Littlejohn et al., 1992), the actinomycete drug resistance translocases cited in this study are predicted to power excretion by a proton-dependent electrochemical gradient. It has been suggested that highly conserved sequences within the amino-terminal regions of these proteins play a role in proton translocation (Rouch et al., 1990), while the less well-conserved C-terminal regions may be involved in drug binding (Paulsen et al., 1996; and references therein) or recognition of a protein-drug complex.

Disruption of mct in S. lavendulae resulted in a 25-fold increase in sensitivity to exogenously added MC, providing evidence that MCT maintains a role in providing drug resistance in S. lavendulae. Although the effect is significant, alternative mechanisms of cellular self-protection clearly continue to operate. This evidently includes MCRA, the novel redox-relay protein that re-oxidizes activated MC in S. lavendulae. It is also likely that unidentified xenobiotic transporters provide an alternative mode of drug transport in the absence of MCT, albeit with lower efficiency.

In order to probe the ability of MCT to transport drug in the presence and absence of the MC binding protein, accumulation of [³H]-MC in E. coli was analyzed. Expression of mct in E. coli resulted in MC-resistant cultures that accumulated lower levels of drug than strains bearing vector control (FIG. 20). Interestingly, strain PJS102 (expressing mct only) accumulates less drug intracellularly than strain PJS103 (expressing mrd and mct) (FIG. 20). Increased drug accumulation in strain PJS100 may lend support to the model of equimolar binding between MRD and MC (Sheldon et al., 1997). Significantly higher levels of drug accumulation in strain PJS100 may be the result of intracellular sequestration of MC by MRD. Accordingly, the presence of MRD could also account for the slightly greater levels of MC accumulation in strain PJS103 (co-expressing mct-mrd) as compared to strain PJS102 (expressing mct alone). Comparable to binding protein-dependent import systems (Miller et al., 1983), the binding of MC by MRD may be rate-limiting in the drug excretion process.

Taken together, these results suggest that cellular protection afforded by MCT is a function of drug transport from the cytoplasm. Interestingly, co-expression of mrd and mct in E. coli led to cultures that are dramatically more resistant to exogenously added drug. While normally required for the transport systems with which they are associated, in many instances binding-proteins are not integral to the process of solute translocation (Higgins et al., 1990). Similarly, the presence of MRD is not required for MC translocation but dramatically enhances drug tolerance. Hence, the binding protein (MRD) may be considered an accessory component, a rather specific adaptation required for optimal drug resistance. The drug-resistance phenotype of E. coli strains expressing mct alone and in combination with mrd along with the MC uptake analysis of these strains provides evidence that MRD and MCT are components of a novel drug transport system. Such a resistance mechanism, sequestering the intact drug for efficient excretion to the environment, represents a unique cellular strategy for self-preservation by the MC-producing organism.

### EXAMPLE 4

Characterization of MitM in the Mitdmycin Biosynthetic Pathway

Since the identification of the anti-tumor antibiotic mitomycin C from cultures of S. lavendulae in the 1950's (Hata et al., 1956), several dozen new mitomycins with varying methylation and amination patterns on the common mitosane skeleton have been isolated. Biosynthetic studies in the 1970's and 1980's showed that the mitomycins are produced by convergent biosynthesis of the precursors 3-amino-5-hydroxy benzoic acid (Anderson et al., 1980), D-glucosamine (Hornemann et al., 1974), carbamoyl phosphate (Hornemann et al., 1975), and S-adenosyl methionine (Bezanson et al., 1971). However, the specific sequence of the reactions required for assembly of these important compounds remains unknown.

The varying activity and toxicity of the anti-tumor mitomycin family of compounds is determined by the methoxy and amino substitution patterns present on the mitosane skeleton (Kunz et al., 1991). To study the late mitomycin biosynthetic steps that give rise to the diversity of mitomycins isolated from nature, molecular genetic methods have been employed to understand the role of the methyltransferases in mitomycin biosynthesis. The mitomycin biosynthetic gene cluster has been identified (Mao et al., 1999a; see Examples 1-3) and a conjugative method for DNA transfer and genetic manipulation of the biosynthetic pathway has been developed (Mao et al., 1999b; Examples 1-3). S-adenosyl methionine is known to label the C7, C9a, and the aziridine O and N methyl groups (Bezanson et al., 1971). The methyltransferase reaction has been putatively assigned (Altschul et al., 1990) to the products of the mitM, mitN and mmcR genes by sequence homology to other known secondary metabolite methyltranferases (Kagan et al., 1994; Lacalle et al., 1991; Madduri et al., 1993; and Shi et al., 1996). A detailed understanding of the sequence of reactions carried out by these enzymes is expected to explain the basis of the diversity of the mitomycins produced. Genetic manipulation of the mitomycin producer, bioassay guided isolation and overexpression of the MitM enzyme has allowed the characterization and conversion of an accumulated intermediate from the methyltransferase mitM deleted mutant, S. lavendulae MM107. In addition to understanding the late mitomcyin biosynthetic steps, the manipulation of the mit/mmc pathway can result in the production of novel mitomycins, e.g., through combinatorial biosynthetic manipulation of the pathway. The results which are described below indicate the sequence of late stage methylations that give rise to the diversity of the isolated mitomycins and evidence the production of a novel mitomycin compound.

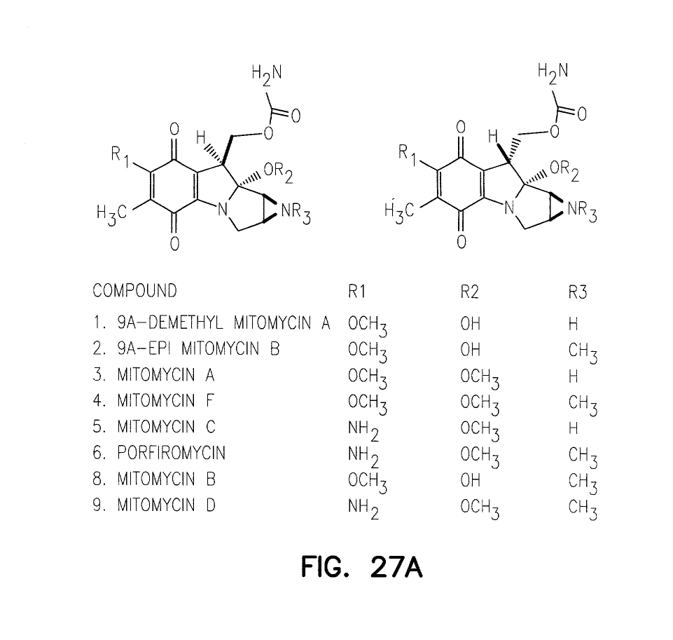

Mitomycin C production was absent in the chromosomally in frame mitM gene deleted S. lavendulae mutant MM107 (see Example 5), however, the observation of a small amount of activity against B. subtilis allowed a bioassay guided isolation of a novel compound, 9a-demethoxy mitomycin A, to take place. The isolation method consisted of applying the crude ethyl acetate extract of the culture broth culture to a Sephadex LH-20 column followed by preparative and analytical reverse HPLC to provide 9a-demethoxy mitomycin A (1) (FIG. 27A) at a yield of 10-30 μg per liter of culture.

Observation of the signals for protons H1, H2, H3α, H3β, Me6, H9, H10, and H10′ in the ¹H NMR of the new compound indicated the mitomycin skeleton was present (Data for 9a demethoxy mitomycin A: ¹H NMR (CD₃CN, 800 MHz). 1.77, (s, C6-methyl), 2.74 (m, H2), 2.81 (d, J=m, H1), 3.31 (dd, J=4.3, 11.3 Hz, H9), 3.39 (d, J=12.8 Hz, H3α), 3.84 (, J=12.8, H3β), 3.95 (s, C7-OCH₃), 4.16 (dd, J=10.4, 11.3 H10′), 4.71 (dd, J=4.3, 10.4, H10); ¹H NMR data for 9-epi mitomycin B (2): (CD₃CN, 800 MHz). 1.78, (s, C6-methyl), 2.22 (s, N-aziridine Me), 2.31 (m, overlapped H1, H2), 3.31 (ddJ=4.9, 9.8 Hz, H9), 3.38 (d, J=12.2 Hz, H3α), 3.84 (d, J=12.2, H3β), 3.96 (s, C7-OMe), 4.06 (dd, J=9.8, 12.2 Hz, H10′), 4.67 (dd, J=4.9, 12.2 Hz, H10)). The presence of the methoxy singlet at 4.0 ppm and the absence of other methoxy or aziridine-N methyl signals at 3.3 ppm and 2.2 ppm, respectively, led to a preliminary structure consistent with 9a-demethoxy mitomycin A. A m/z of 374.0074 (1.9 ppm of calc'd) from HRESIMS of [M+K]⁺ confirmed the formula for this structure. The ¹³C NMR signals for the carbon skeleton was observed by HMQC and HMBC experiments due to difficulties in obtaining sufficient signal from directly detected ¹³C NMR experiments (¹³C NMR (CD₃CN, 800 MHz, inversely detected by HMBC and HMQC, ppm, atom). 37.4, C1 , 33.4 m C2, 49.6, C3, 152.0, C4, 184, C5, 7.5, C6a (methyl), 125, C6, 160.4, C7, 114.2, 62.0, C7a (O-methyl), C8a, 103, C9a. 49.4, C9, 157.9, C10a (carbamoyl carbonyl), 63.0, C10. C8 was not detected by HMBC). In addition to providing the ¹³C NMR spectrum, the heteronuclear correlation experiments demonstrated the presence of the quinone moiety, the connectivities for the 7-methoxy and C6 methyl groups to the aminoquinone group, and the presence of the carbamoyl carbon from a H10 to C10a correlation.

Determination of the stereochemistry for the C9-C10 bond was first attempted by difference nOe, NOESY and ROESY experiments, however, the lack of a clear nOe effect between the protons on C10 and the H1, H2 and H3 protons prevented assignment of stereochemistry. Overexpressed MitM from E. coli (Example 5) was used to convert 9a-demethoxy mitomycin A to an N-aziridine methylated product. The 2.2 ppm methyl signal and the upfield shift of the H1 and H2 signals from 2.90 and 2.85 to merge at 2.5 ppm are consistent with an N-aziridine methyl group in the mitomycin series of compounds. By thin layer chromatography (silica gel, 9:1 CHCl₃:CH₃OH) it was apparent the product (R_{f}=0.21) was not mitomycin B (R_{f}=0.26), and thus must be 9-epi mitomycin B with C9-C10 in the β configuration. The stereochemistry of 1 and 2 at the C9a, C1 and C2 carbons were assigned based on biosynthetic consistency with previously isolated mitomycins (Hornemann et al., 1985).

The activity of MitM upon 9a-demethoxy mitomycin A was examined to identify the role of this enzyme in the mitomycin C biosynthetic pathway. MitM was found to have a pH optimum of 8 (above a certain pH the SAM substrate decomposes, while below pH 7 the mitomycins are known to be unstable). As described above, MitM was shown to methylate specifically the aziridine nitrogen of 9a-demethoxy mitomycin A while leaving the 9a oxygen untouched. Addition of a methyl group was confirmed by the HRESIMS of 2 which provided a m/z=[M+Na]⁺ 372.1186 (3.9 ppm of calc'd). Kinetic experiments with varying amounts of 9a-demethoxy mitomycin A at 500 μM SAM allowed determination of a Km of 263 μM and a k_{cat} of 0.11 s⁻¹. For SAM the Km of 30 μM, and a k_{cat} 23 μM were determined, however, these experiments took place with 400 μM mitomycin A due to limited amounts of 9a-demethoxy mitomycin A. Using the same conditions that provided activity for mitomycin A and 9a-demethoxy mitomycin A enzymatic conversions, no conversion products were observed for mitomycins B, C, D, and G.

Discussion

The identification and sequencing of the mitomycin C biosynthetic pathway allowed the assignment of methyltransferase functions to the proteins encoded by the genes mitM, mitN, and mmcR (Mao et al., 1999a). A conjugative transfer method for S. lavendulae was used to create mutants containing either deleted or insertionally disrupted versions of these genes on the S. lavendulae chromosome (Mao et al., 1999b). Examination of the mitM deleted mutant, MM107, for accumulated intermediates led to the isolation and characterization of the new mitomycin, 9a-demethoxy mitomycin A. The presence of a demethylated mitomycin was expected from the previous assignment of methyltransferase activity to mitM based on homology to other secondary and primary metabolite methyltransferases. The identification of this novel compound reaffirms that knockout mutants in the mitomycin C biosynthetic pathway are an effective strategy to determine the biosynthetic sequence of this important anti-cancer agent.

The isolated intermediate 9a-demethoxy mitomycin A can potentially be methylated at either the 9a oxygen or the aziridine nitrogen by SAM. Reacting 9a-demethoxy mitomycin A with MitM provided 9-epi mitomycin B, indicating that this methyltransferase specifically acts upon the aziridine nitrogen. A further probe of MitM activity was carried out with the mitomycins A, B, C, D and G. Only mitomycin A was methylated at the aziridine nitrogen by MitM to produce mitomycin F. While mitomycin C also has a free aziridine nitrogen that can potentially be methylated to form porfiromycin this was not observed in the reaction with MitM. Thus, the reactivity of MitM appears to be general with regard to the aziridine position of the mitomycins with a C7-methoxy group such as mitomycin A and 9a-demethoxy mitomycin A, but the presence of the C7-amine of mitomycin C prevents this activity. Due to lack of a suitable substrate in the mitomycin B series of compounds (α C9-10 bond configuration, unmethylated aziridine) the specificity of MitM with regards to this stereochemistry could not be determined.

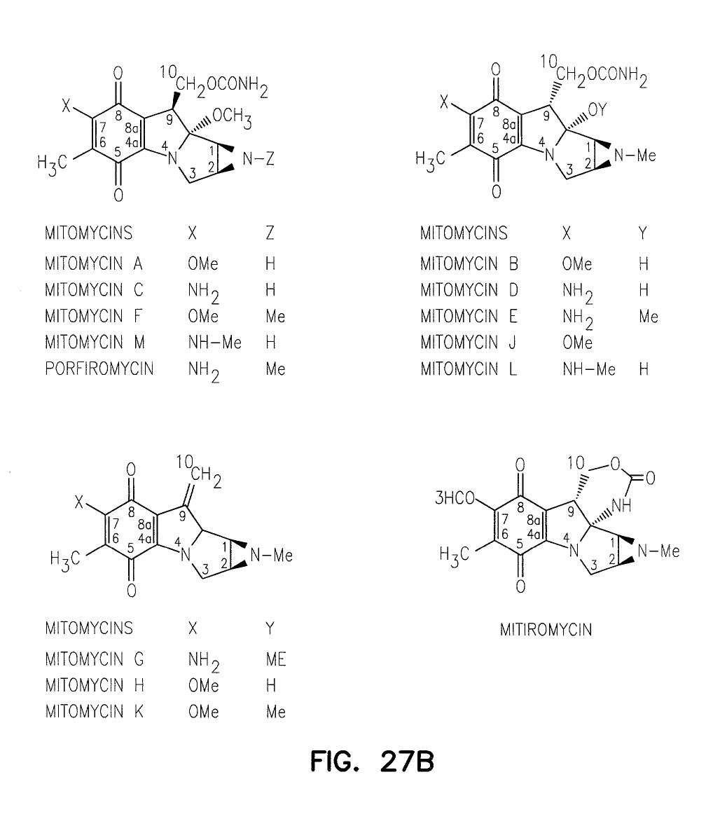

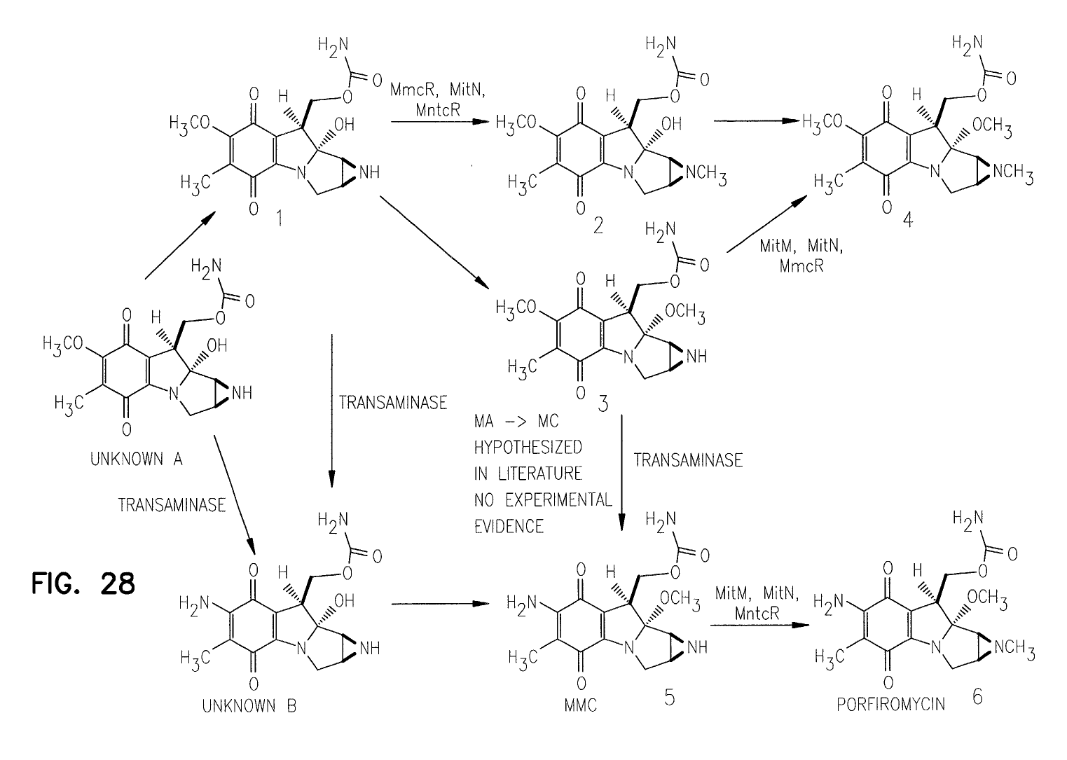

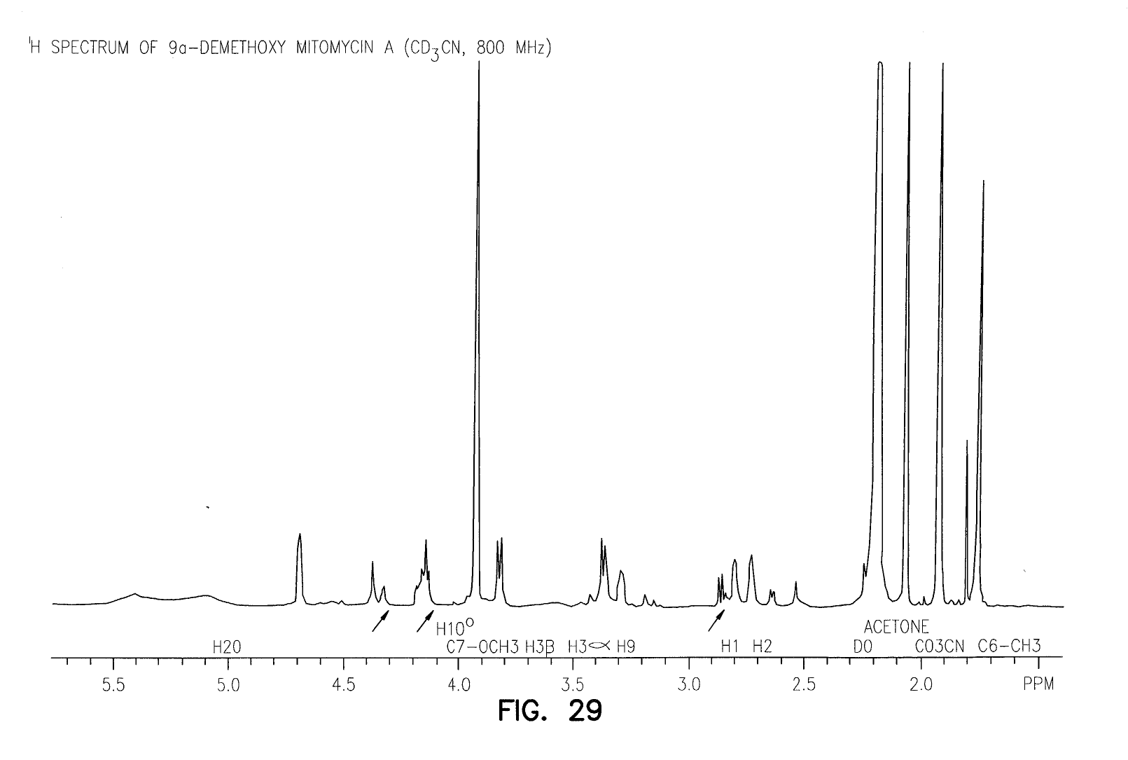

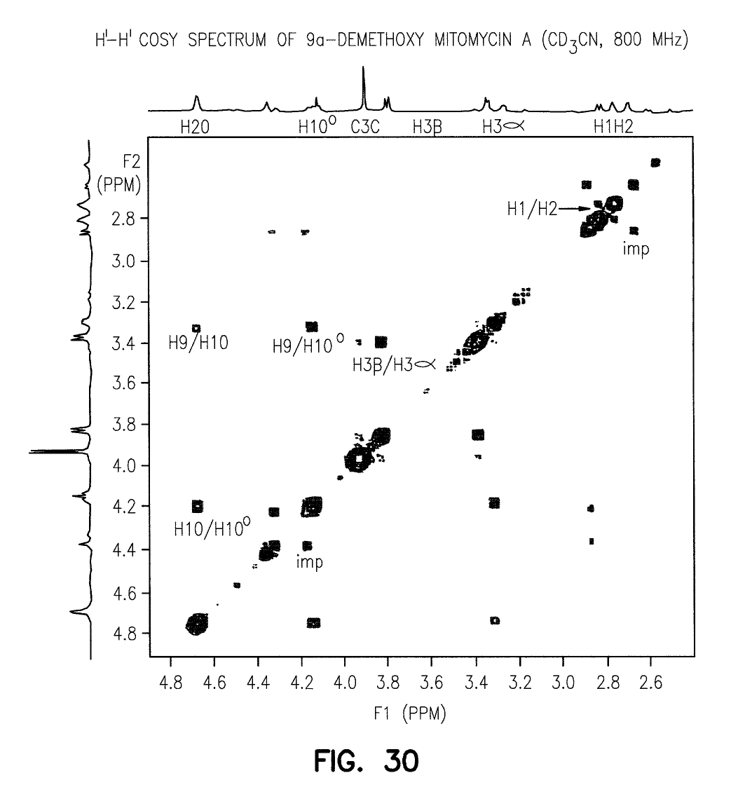

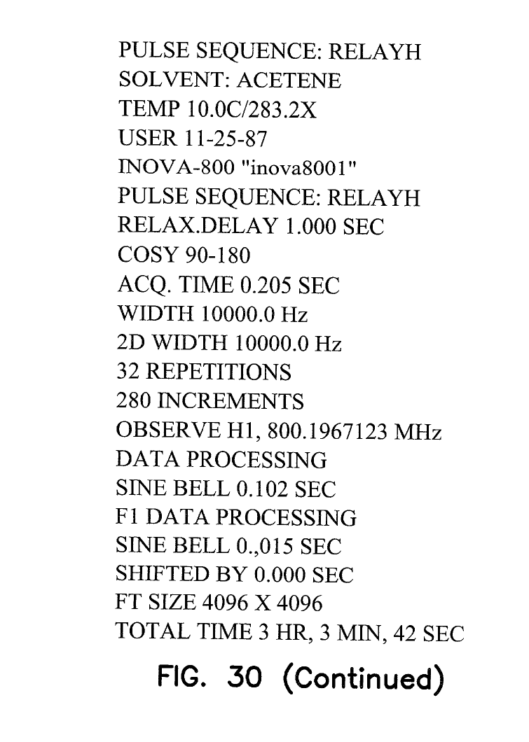

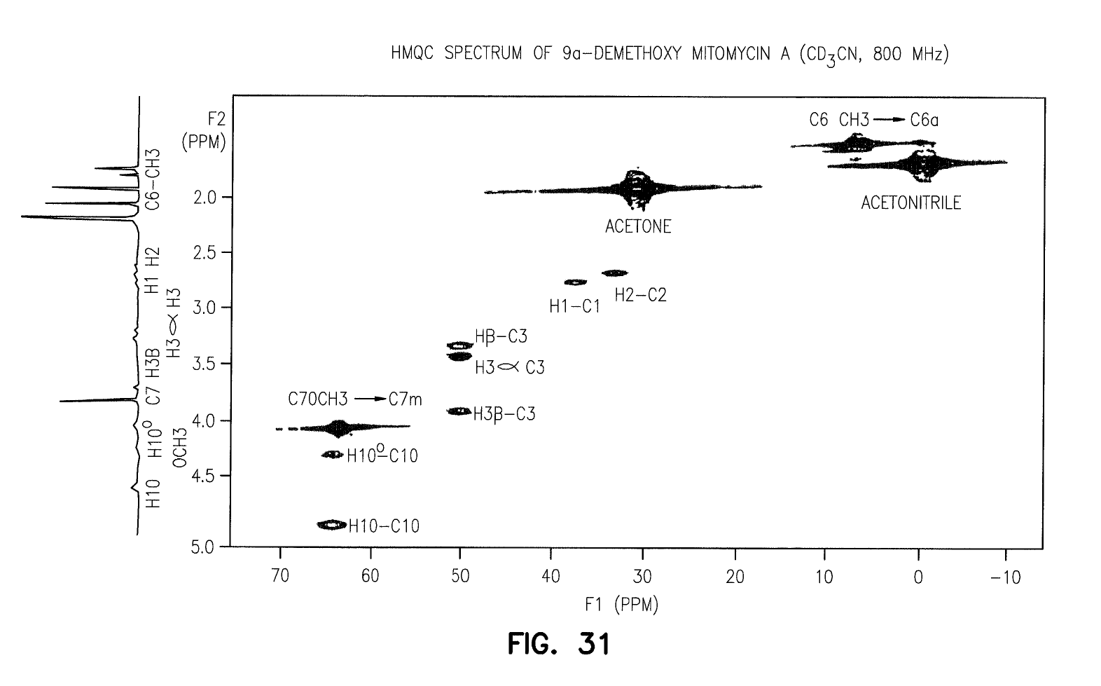

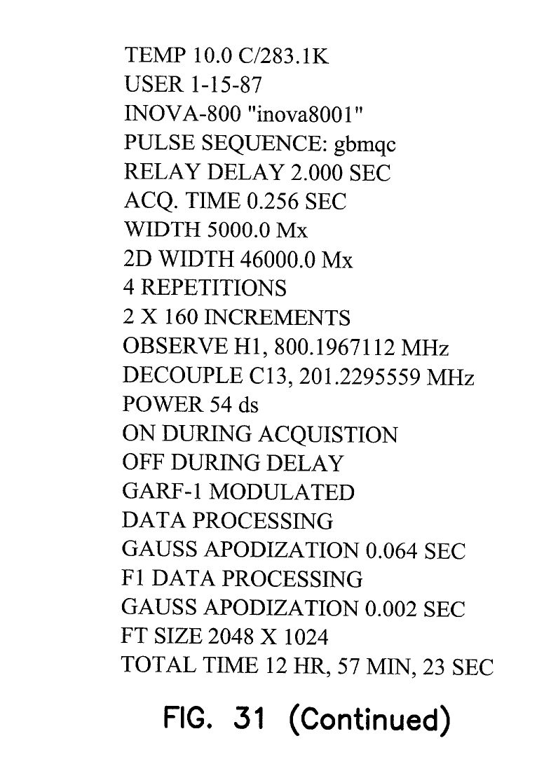

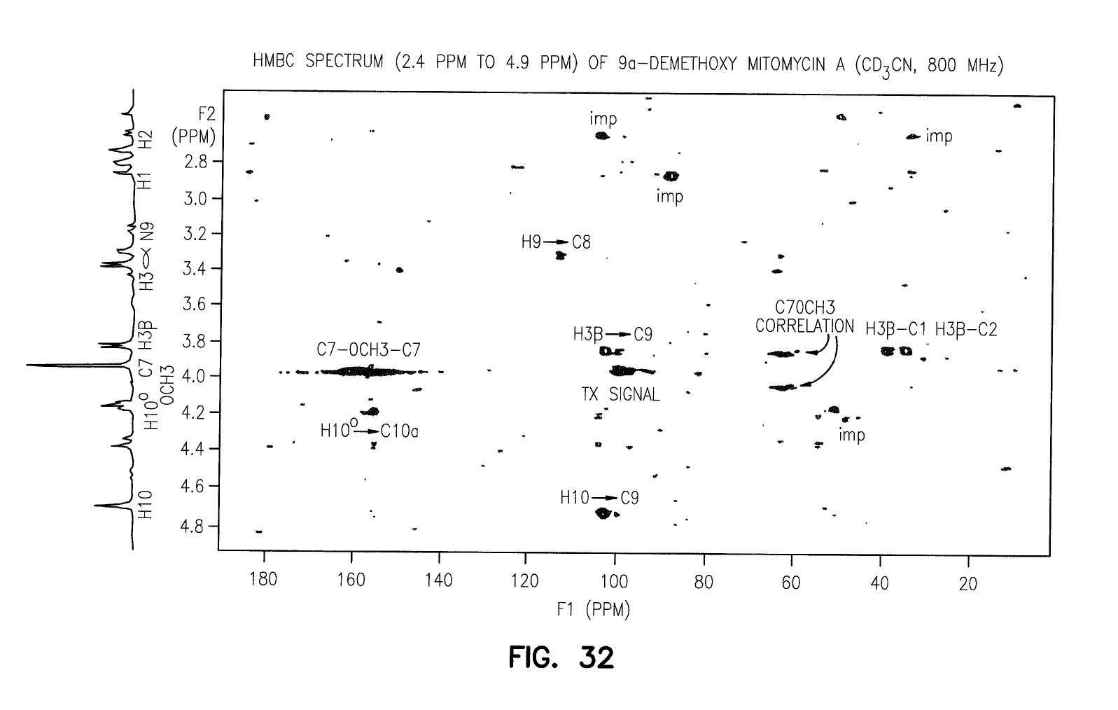

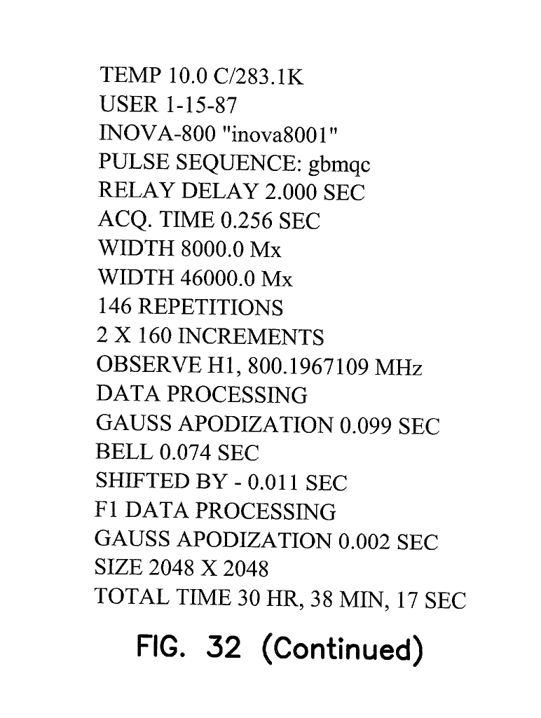

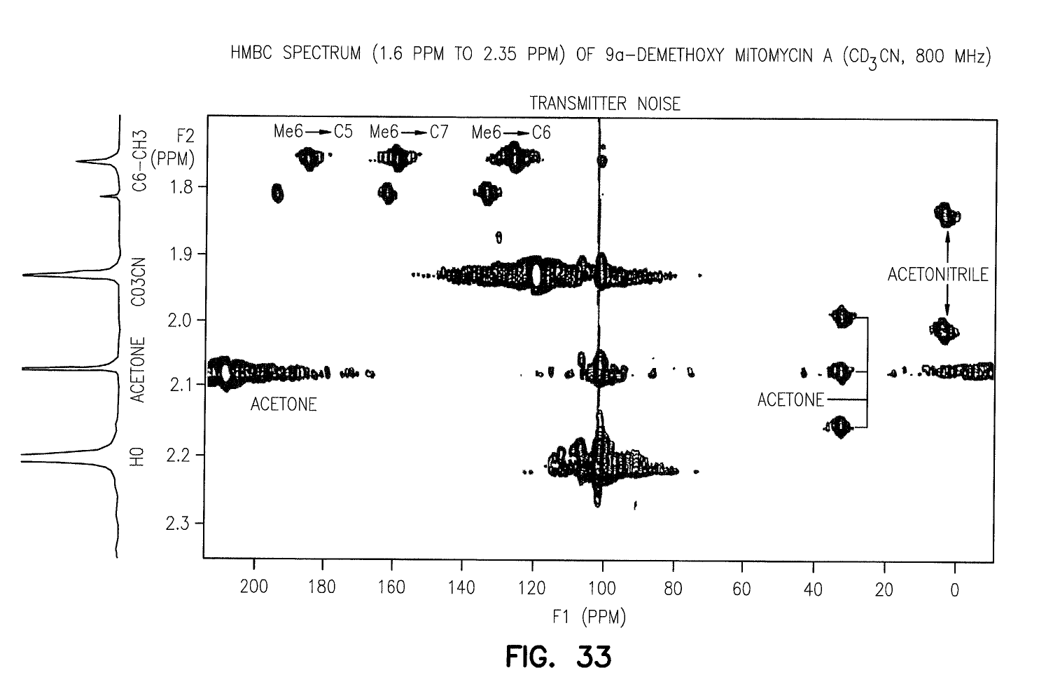

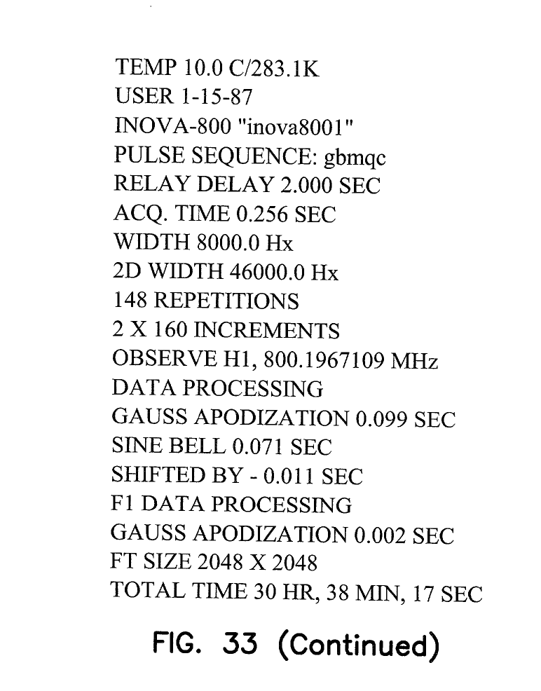

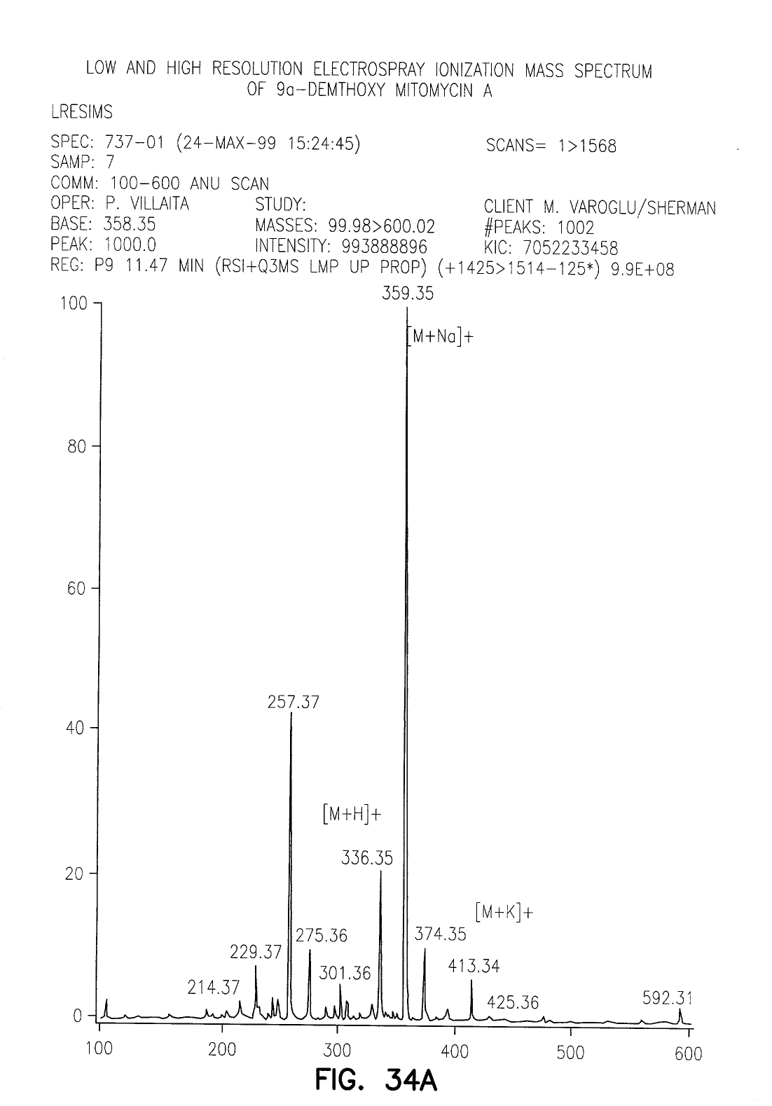

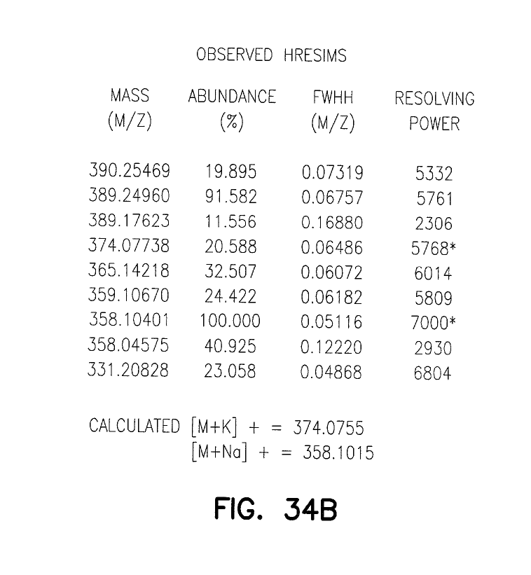

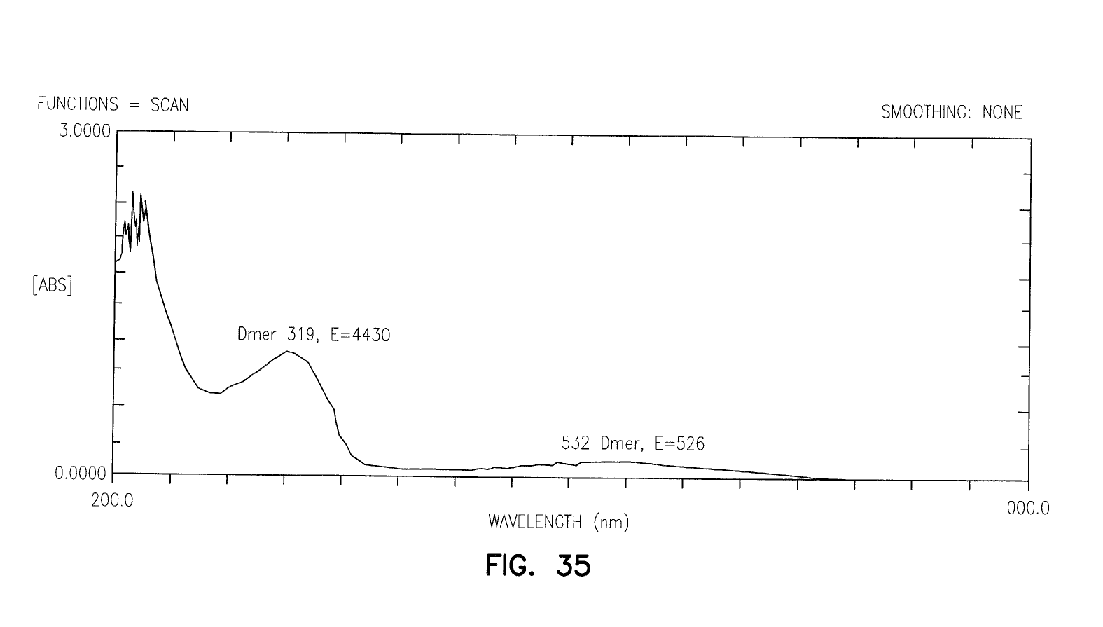

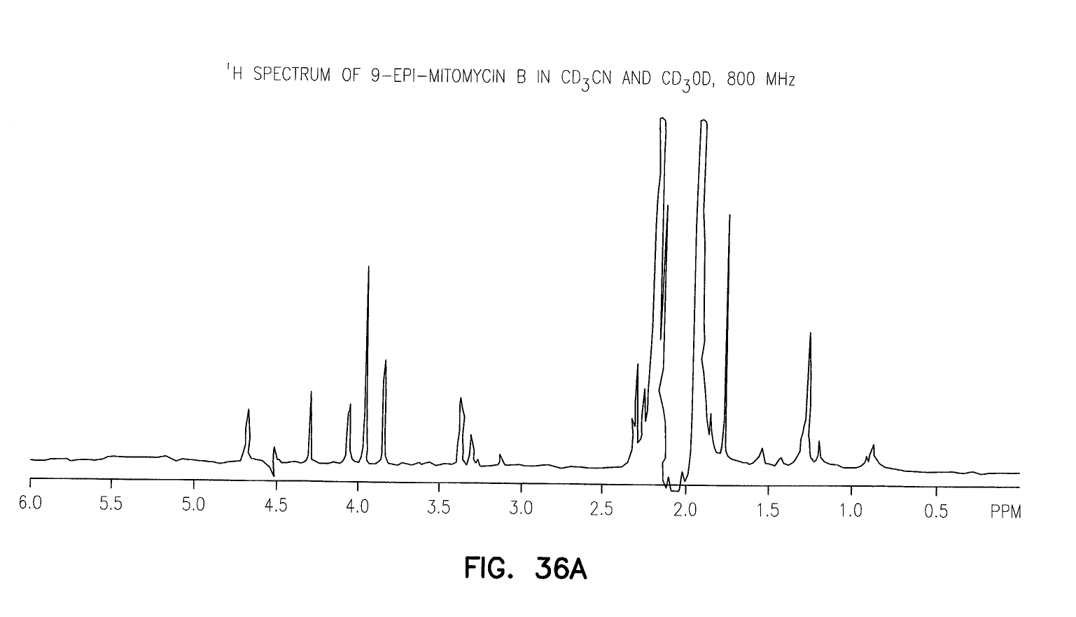

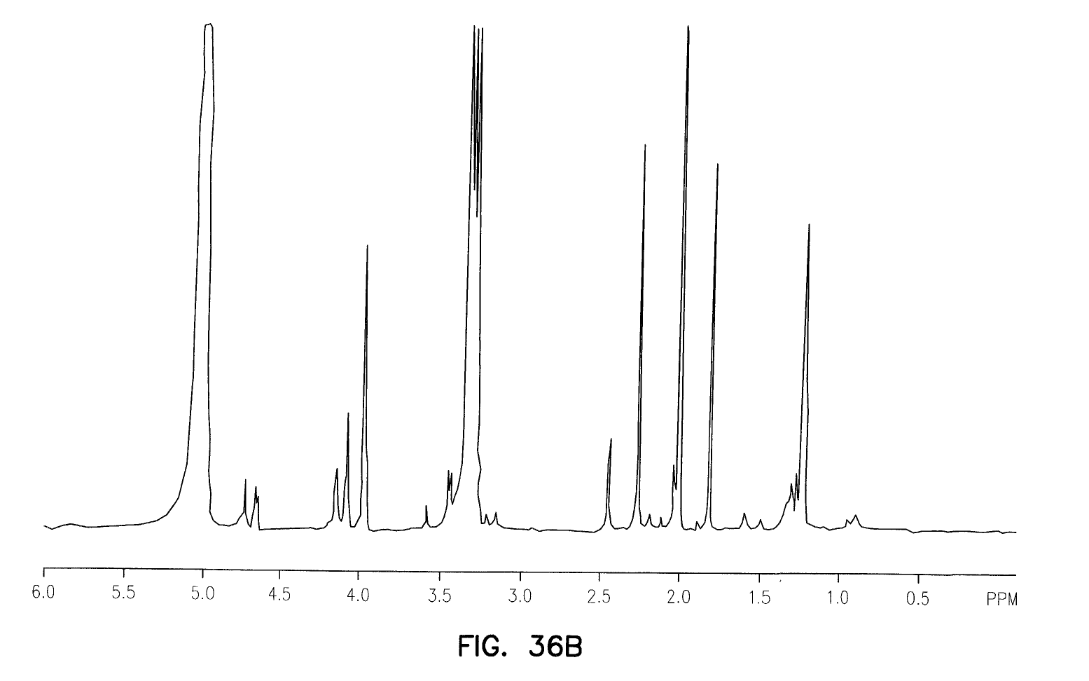

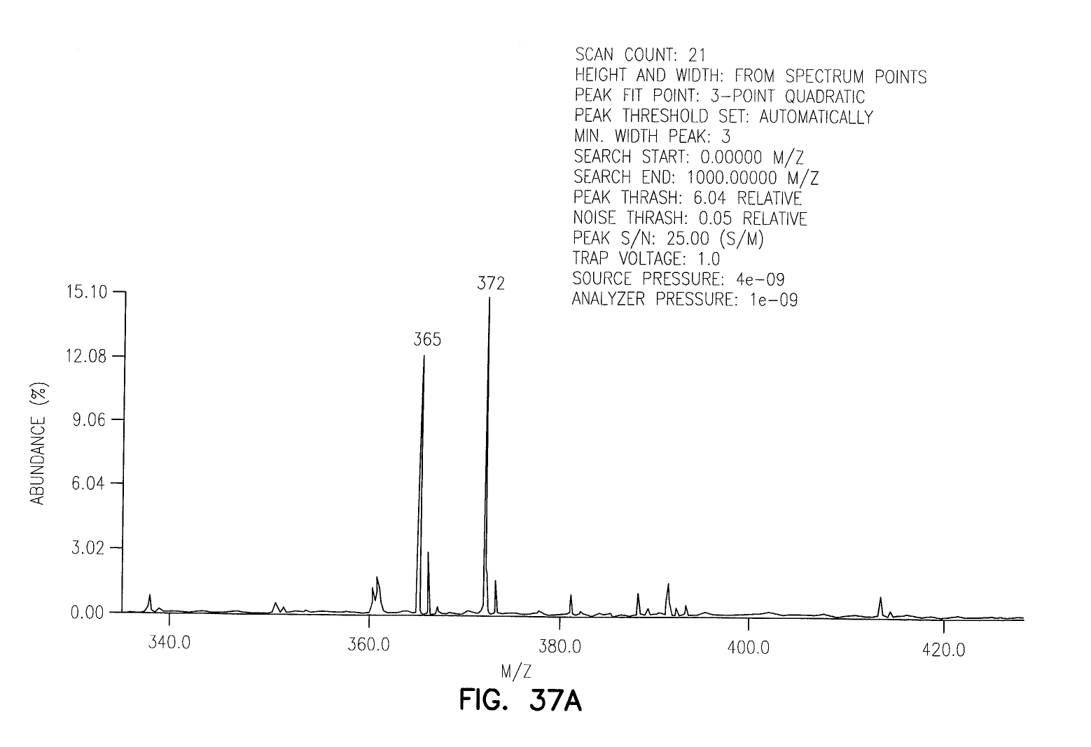

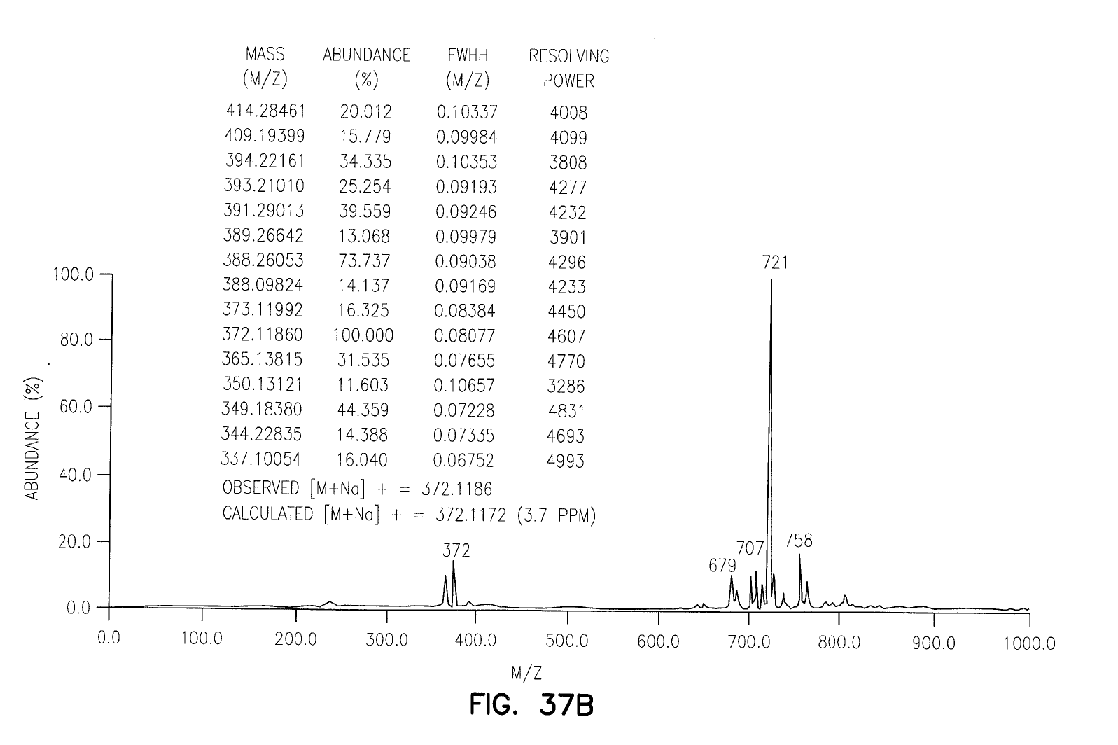

It has been observed that mitomycin A titers first rise and then fall as mitomycin C titers increase (Hornemann, 1981). This implied that mitomycin C is produced from mitomycin A. To date there has no been direct biochemical evidence for this transformation. From the results described herein, it was found that affecting the enzyme that converts mitomycin A to F and 9a-demethoxy mitomycin A to 9-epi mitomycin B led to the abolition of mitomycin C production. When the late methylations for the mitomycins with C9 α stereochemistry are examined, scheme 1 (FIG. 28), an early split from a putative demethylated precursor Unknown A can be hypothesized. This putative precursor can be methylated at the C7 position to form 9a-demethoxy mitomycin A and enter into the mitomycin A series of compounds, or an amine may added at the C7 position to form Unknown B and start the mitomycin C group of compounds. Whether the split occurs early as shown in scheme 1, or if there is an aminotransferase that acts upon the either mitomycin A or one of its related C7 methoxy compounds, is not known.

Thus, it appears that MitM is responsible for methylating the aziridine nitrogen to convert 1 to 2 while also converting mitomycin A to F. Hence, the molecular genetic manipulation of the mitomycin biosynthetic pathway has provided information on the sequence of the reactions involved in mitomycin C biosynthesis. Compound 1 has not been previously synthesized or isolated (Yoda et al., 1993) and 2 is only known from synthesis using mitomycin B as the starting material (Kasai et al., 1989).

### EXAMPLE 5

### Genetic and Enzymnatic Analysis of Two Aziridine N-methyltransferases (MitMn) in the Mitomycin Biosynthetic Gene Cluster

Mitomycin C (MC) is a clinically important anti-tumor antibiotic and has been widely used for combination cancer chemo- and radio-therapy for more than three decades (Henderson, 1993). In an effort to select more potent and low toxicity anti-tumor drugs, chemists and microbiologists have synthesized various mitomycin analogs (Kasai et al., 1995). The first isolated mitomycin antibiotics were mitomycin A (MA) and mitomycin B (MB) in 1956 in the fermentation of S. lavendulae (Hata et al., 1956). In the following year, Wakaki et al. (1958) found mitomycin C (MC) in the same culture broth when it did not produce MA and MB. The mitomycin family was further enriched by identification of a group of minor mitomycin components from the fermentation broth in the ensuing years (Shirahata et al., 1981). These compounds had a common heterocyclic ring system: the mitosane core, while they had different modifications (e.g., methylation, amination, or both) at N-1a, C-7 and C-9 positions (FIG. 27B). The methyl groups at these positions had been demonstrated to be derived from L-methionine since radio-labeled L-[¹⁴CH₃] methionine can specifically label the O- and N-methyl groups in MA while antibiotic production was inhibited by the methionine antagonist D,L-ethionine (Kirsch et al., 1964; Bezanson et al., 1971). Another experiment in the early 1970s had shown that one of the demethylated derivative of MC can be converted to MA by the cell-free extracts of S. lavendulae when supplied with exogenous L-methionine, ATP and Mg2+, while at the same condition, the C10 decarbamoylated form can not (Nishikohri et al., 1975). These experiments not only demonstrated the existence and involvement of methyltransferases in MC biosynthesis, but also indicated those enzymes had some degree of substrate specificity.

The final step for methyl transfer utilizing L-methionine as the methyl donor is usually catalyzed with a group of enzymes called S-adenosylmethionine (SAM)-dependent methyltransferases. In the presence of ATP, Mg2+, L-methionine was first converted by synthetase to SAM. These methyltransferases then transfer the methyl group from SAM to either macromolecules such as proteins, nucleic acids (RNA or DNA), phospholipids, polysaccharides, or small molecules such as drugs, hormones, and neurotransmitters with the formation of methyl ester, methyl ether, methyl thioether, methyl amine, methyl amide, and other derivatives (Weinshilboum et al., 1999). Though there existed three loosely conserved SAM or S-adenosyl-homocysteine binding motifs within the enzymes, methyltransferases do not display the same degree of sequence homology as had been fond among the cytochromes P450, and no similar overarching classification system had been developed (Kagan et al., 1994). The most commonly found small molecule methyltransferases were O-methyltransferase which has been widely found in both eukaryotes (such as catechol O-methyltransferase; see Lundstrom et al., 1995) and prokaryotes. Most of the reported macrolide antibiotic biosynthetic methyltransferases were revealed to be in this category. These genes includes: eryG for erythromycin (Haydock et al., 1991), tcmN for tetracenomycin (Summers et al., 1992), dnrK for carminomycin (Madduri et al., 1993), dauK for daunomycin (Dickens et al., 1995), orf14 for rifamycin (August et al., 1998), aveD for avermectin (Ikeda et al., 1999),fkbM for FK506 (Motarnedi et al., 1996), mdmC for midecamycin (Hara et al., 1992), and dmpM for puromycin (Lacalle et al., 1991). Small molecule SAM dependent N-methyltransferase is quite common in mammalian cells in the metabolism of many endogenous neurotransmitters and hormones, e.g., phenylethanolamine N-methyltransferase (Vance et al., 1998), histamine N-methyltransferase (Okinga et al., 1995), and glycine N-methyltransferase (Ogawa et al., 1998). Though N-methyltransferase genes were also proposed in antibiotic biosynthetic gene clusters (pur5 in puromycin gene cluster, see Tercero et al., 1996), spcM for spectinomycin biosynthesis (AAD28488), tylM1 in tylosin cluster (Gandecha et al., 1997), none of their functions has been confirmed

Three SAM dependent methyltransferases (MmcR, MitM and MitN) were assigned in the MC biosynthetic gene cluster because of the three conserved SAM binding motifs within their protein sequences (Mao et al., 1999). Database analysis revealed that MmcR showed strong sequence similarity with a group of antibiotic O-methyltransferases, whereas both MitM and MitN are most closely related to a group of plant δ-(24)-sterol C-methyltransferases as well as some antibiotic biosynthetic O-methyltransferases (Mao et al., 1999). Considering that both the O- and N-methyl groups in mitomycin molecules were derived from L-methionine and there were three SAM dependent methyltransferases available in the MC cluster, localization of the detailed functions of these methyltransferases in MC biosynthesis will provide insight into determining the details of MC biosynthesis and the substrate specificity of these enzymes.

Material and Methods

Bacterial Strains and Cloning Vectors

E. coli DH5α (Table 5) was routinely used as a cloning host, and propagated on LB media E. coli S17-1 was used as a conjugation host for gene transfer from E. coli to S. lavendulae. Conjugation was performed on AS1 media and double crossovers were selected on R5 media as described previously (Mao et al., 1999). E. coli BL21 (DE3) was the protein overexpression host in NZYM media.

TABLE 5
Bacterial strains and plasmids.

| Strains or | | |
|---|---|---|
| Plasmids | Relevant Characteristics^{a} | Reference |
| E. coli strains | | |
| DH5α | F recA φ80 dlacZ ΔM15 | Gibco |
| | | BRL |
| S17-1 | Contains RP4 integrated into the | Mazodier |
| | chromosome | et al., |
| | | 1989 |
| BL21 (DE3) | F ompT hsdS gal dcm | Novagen |
| S. lavendulae | (DE3) | |
| NRRL 2564 | Wild type MC producer | ATCC |
| MM106 | mitM insertional disruption mutant of NRRL | This Study |
| | 2564 | |
| MM107 | mitM in frame deletion mutant of | This Study |
| | NRRL 2564 | |
| MM108 | mitN in frame deletion mutant of | This Study |
| | NRRL 2564 | |
| Plasmids | | |
| pET28b | Kan^{R} , Protein expression vector | Novagen |
| pUC119 | Ap^{R} lacZαMCS, E. coli cloning vector | Yanisch- |
| | | Peron et |
| | | al., 1985 |
| pKC1139 | AM^{R} lacZαMCS oriT | Bierman |
| | rep^{ts} | et al., |
| | | 1992 |
| pDHS7608 | mitMN containing 2.5 kb BamH1 | This study |
| | fragment in pUC119 | |
| pDHS7705 | mitM disruption construct in pUC119 from | This study |
| | pDHS7608 | |
| pDHS7706 | mitM disruption vector in pKC1139 from | This study |
| | pDHS7705 | |
| pDHS7707 | mitM deletion construct in pUC119 | This study |
| pDHS7708 | mitM deletion vector in pKC1139 from | This study |
| | pDHS7707 | |
| pDHS7709 | mitN deletion construct in pUC119 | This study |
| pDHS7710 | mitN deletion vector in pKC1139 from | This study |
| | pDHS7710 | |
| pDHS7801 | MitM overexpression vector | This study |
| pDHS7802 | MitN overexpression vector | This study |

^{a}AM^{R} , apramycin resistance;
Ap^{R}, ampicillin resistance;
Kan^{R}, neomycin resistance;
rep^{ts}, temperature-sensitive replicon in Streptomyces

pUC119 was the cloning vector in E. coli. pKC1139, an E. coli-Streptomyces conjugative shuttle vector was used to create disruption mutants in S. lavendulae. Plasmid pET28(b) was used for protein overexpression in E. coli BL21 (DE3).

DNA Manipulation

The mitM insertional disruption vector pDHS7706 was constructed as follows: the mitMN genes-containing plasmid pDHS7608 was digested with Bpu11021, blunt-ended and then ligated with the 1.4 kb ApaL1-HindIII (aphII) fragment from pFD666 to create pDHS7705. The insertion of the aphII gene in Bpu11021 results in the disruption of mitM. The 3.9 kb BamHI insert from pDHS7705 was then moved to the shuttle vector pKC1139 to generate pDHS7706.

Three pairs of primers were used to construct the mitM and mitN in-frame deletion vectors pDHS7708 and pDHS7710. For the mitM in-frame deletion vector pDHS7708, primer f1: 5′-GC TCT AGA TCT ACG TCT CCC GCG-3′ (XbaI; SEQ ID NO:146) and primer r1: 5′-GC CTC GAG CAT GGA CGA TCC CTC TCG-3′ (XhoI; SEQ ID NO:147) were used to generate a 1.1 kb mitM upstream fragment (MTF11) by introducing an additional XhoI site right after the mitM translational start codon. A 2.1 kb mitM downstream fragment (MTF23) was amplified with primer f2: 5′-GC CTC GAG CCG GGA AAG TGA GCG GCA-3′ (XhoI; SEQ ID NO:148) and primer r3: 5′-GC AAG CTT CGG CAT CAC GCG CCA-3′ (HindIII; SEQ ID NO:149) by introducing a XhoI site just upstream of the mitM translational stop codon. MTF11 and MTF23 were then digested with XbaI/XhoI and XhoI/HindIII, respectively, and ligated to the XbaI/HindIII digested pUC119 to create pDHS7707. In this case, mitN was left intact, while the whole ORF of mitM was replaced by a XhoI site. The 3.2 kb insert from pDHS7707 was then subcloned into the XbaI/HindIII site of pKC1139 to generate pDHS7708. Similarly, a 1.9 kb mitN upstream fragment (MTF12) and a 1.3 kb downstream fragment (MTF33) were amplified with two pairs of primers: primer f1 and primer r2: 5′-GC CTC GAG CGT CAT GCC OGCT CAC TTT-3′ (XhoI; SEQ ID NO:150) for MTF12 and primer f3: 5′-GC CTC GAG TAG GGC TCC CAC GGG AAG-3′ (XhoI; SEQ ID NO:151) and primer r3 for MTF33. Three way ligation of the XbaI/XhoI digested MTF12, XhoI/HindIII digested MTF33 and the XbaI/HindIII digested pUC119 resulted in pDHS7709, in which the mitN ORF was replaced with a XhoI site while mitM was left intact. pDHS7710 was created by subcloning the 3.2 kb XbaI/HindIII insert from pDHS7709 into pKC1139.

For the construction of the MitM and MitN overexpression vector, an NdeI site was introduced at the translational start codon of mitM with primer f4: 5′-GGG ATC GCA TAT GCC GCA CTC CGA GCT GTC-3′ (NdeI; SEQ ID NO:152), and mitN with primer f5: 5′-GTG AGC CAT ATG ACG GAA ACC GCG TCC GC-3′ (NdeI; SEQ ID NO:153). With pDHS7608 as template, and M13 /pUC reverse sequencing primer as the other PCR primer pair, a 2.31 kb fragment (for MitM) and a 1.45 kb fragment (for MitN) were amplified, respectively. The resulting fragments were then digested with NdeI/HindIII and subcloned into the corresponding sites of pET28b to generate pDHS7801 (for MitM) and pDHS7802 (for MitN). In both cases, the overexpressed proteins were engineered to include an N-terminal 6×His tag.

Creation of Disruption Mutant Strains of S. lavendulae

The mitM insertional disruption mutant S. lavendulae MM106 was created by conjugating the mitM disruption vector pDHS7706 into wild type S. lavendulae NRRL 2564 following the protocol described in Mao et al. (1999). An apramycin, kanamycin double resistant conjugant was selected and propagated on R5 plates at 39° C. for several generations to select for the double crossover mutants. Kanamycin resistant and apramycin sensitive colonies were checked by Southern hybridization to have the correct double-crossover genotype.

A two-step recombination strategy was used to create the mitM and mitN in-frame deletion mutants MM107 and MM108. In these experiments, pDHS7708 and pDHS7710 were conjugated into MM106, the mitM insertional disruption mutant. Following by the selection of kanamycin and apramycin sensitive colonies from the corresponding double antibiotic resistant conjugants, mitM and mitN were successfully deleted from the chromosome. The deletion of mitM in MM107 and mitN in MM108 were confirmed by Southern hybridization.

Overexpression and Purification of MitM and MitN

pDHS7801 and pDHS7802 were transforned into E. coli BL21 (DE3) strain for the overexpression of MitM and MitN, respectively. E. coli cells were grown at 37° C. in NZCM broth containing 50 μg/ml kanamycin until OD₆₀₀=0.6. Expression of MitM (or MitN) was induced by adding IPTG to a final concentration of 0.1 mM and reducing the temperature to 25° C. After incubating the culture overnight, cells were harvested, suspended in ice-cooled lysis buffer (50 mM NaH₂PO₄, pH 8.0; 300 mM NaCl; 10 mM imidazole), and lysed by sonication (6×10 seconds with 10 second pauses at 200-300 W). The supernatant collected by centrifuging the lysate at 10,000 g at 4° C. was used to pass through a Qiagen Ni-NTA column as specified by the manufacturer. The column was eluted twice with wash buffer (50 mM NaH₂PO₄, pH 8.0; 300 mM NaCl; 20 mM imidazole) followed by dissolving the protein in elution buffer (50 mM NaH₂PO₄, pH 8.0; 300 mM NaCl; 250 mM imidazole). The protein eluted from the column was then precipitated with 60% ammonia sulfate, desalted by passing through a Pharmacia PD-10 column, and dissolved in 50 mM pH7.4 potassium buffer with 1 mM PMSF. The protein concentration was determined using the Bradford Assay with BSA as the standard. The final concentration of MitM is 0.38 mg/ml and MitN is 0.71 mg/ml.

Enzyme assays

Enzymatic conversion of MitM (and MitN) with different substrates was carried out in a total volume of 100 μl in 50 mM potassium phosphate buffer (pH 7.4), containing 100 μM S-adenosyl-methionine (SAM), an appropriate quantity of substrate, and 5 μg of purified enzyme. The reactions were incubated at 30° C. for 30 minutes and were terminated by twice equal volume of ethyl acetate extraction. The resulting organic extracts were combined, vacuum dried and resuspended in 20 μl methanol for further TLC or HPLC analysis (Mao et al., 1999).

Product Identification

Both MitM and MitN can convert MA to a new product, with the TLC and HPLC behaviors identical to MF. For further confirmation, the products isolated and purified from several standard enzyme reactions by HPLC were analyzed by ¹H NMR.

Kinetic Measurement

The kinetic data for the conversion of MA to MF by MitM and MitN were obtained by detecting the ME production in 313 nm with HPLC under conditions of initial velocity (5-20 minutes) with 1.9 μg MitM or 1.4 μg MitN. Kinetic parameters (Vₘₐₓ and Km) were determined for both substrate MA and SAM from the Lineweaver-Burk double reciprocal plot. For MA, reactions were carried out at a fixed amount of SAM (200 μM) with variable amount of substrate MA (30-300 μM), while the parameters for variable SAM (40-400 μM) were measured at the fixed concentration of MA (200 μM). The calculations were carried out using Kcat computer program by fitting the data to a Michaelis-Menton equation by linear regression.

Results

Creation of mitM and mitN Deletional Mutants

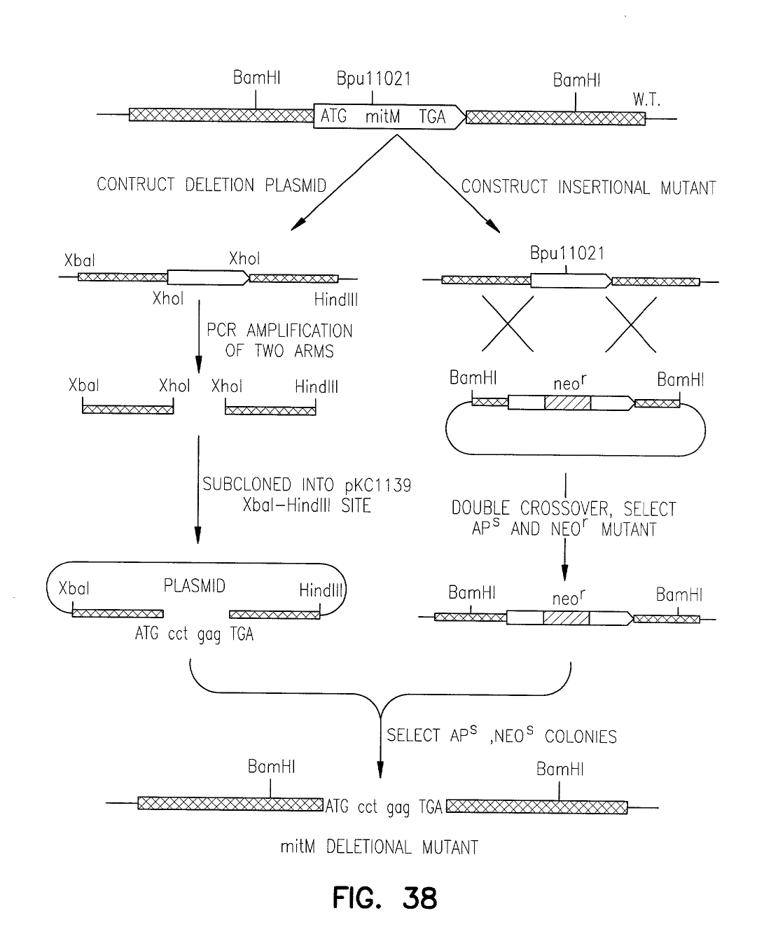

The chromosomal copy of mitM gene was disrupted by inserting a neomycin resistance marker in the middle of its open reading frame. The resultant mutant strain, MM106, abolished MC production. However, unlike the early MC biosynthetic gene (mitA) knockout mutant which totally blocked mitomycin compounds production, MM106 still produced some colorful bioactive mitomycin intermediates (data not shown). Since mitM and mitN are the last two genes within a 14-gene cluster which had the same transcription direction (Mao et al., 1999), insertional disruption of mitM probably affects the downstream mitN gene expression. To avoid such polar effect, a mitM in-frame deletion mutant MM107 was created from MM106 followed by a two step recombination in which the whole mitM ORF was replaced with two amino acids (FIG. 38). Similarly, a mitN in-frame deletion mutant, MM108, was also created from MM106 with the same strategy. Both mutants were selected based on the phenotype changing from kanamycin, apramycin double resistant to double sensitive, and confirmed by the correct band shifts with Southern hybridization (data not shown).

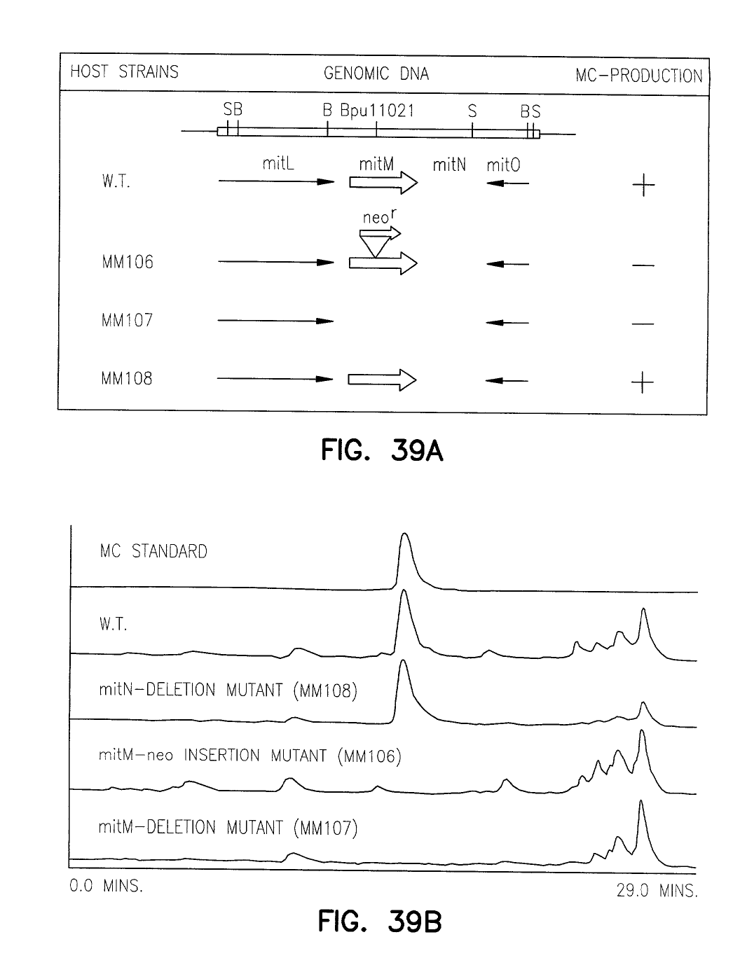

As shown in FIG. 39, the mitM deletion mutant (MM107) does not produce MC, while the mitN clean deletion mutant (MM108) slightly increases MC production. These results indicate that MitM is a key enzyme in MC biosynthesis, while MitN probably serves in the production of shunt metabolites.

Overexpression and Purification of MitM and MitN in E. coli

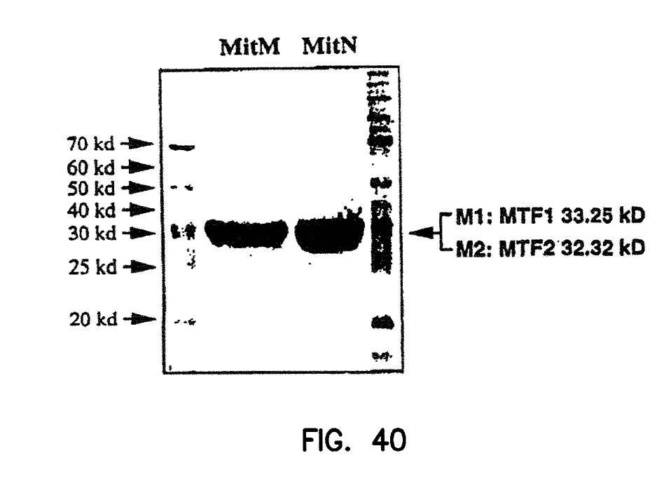

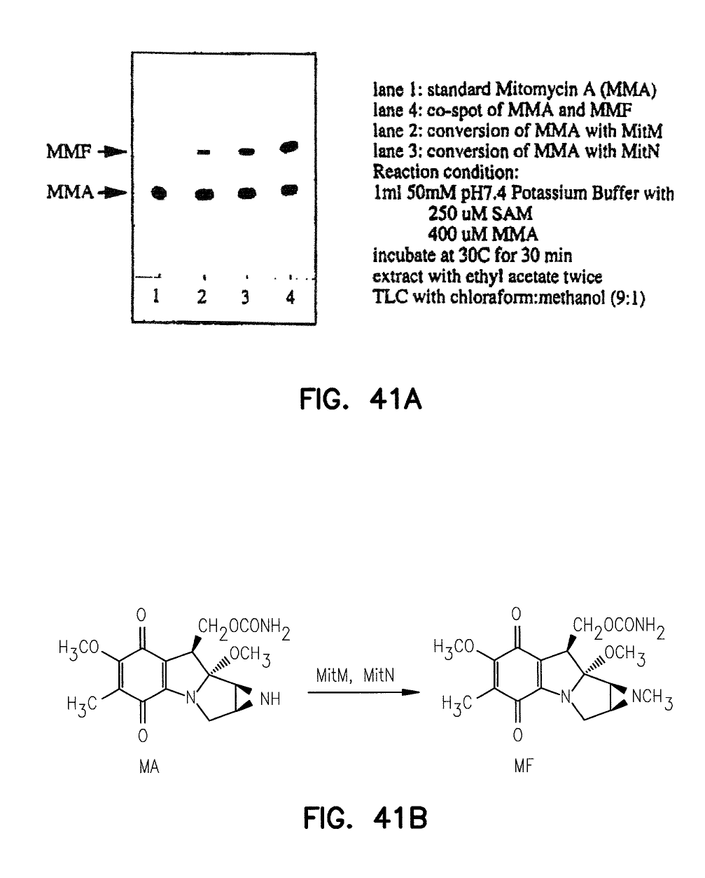

MitM is a hydrophilic protein (deduced pI=4.66) which comprises of 283 amino acids with a calculated molecular weight (MW) of 31 kD. Similarly, MitN consists of 275 amino acids with a deduced MW of 30 kD and a deduced pI of 4.59. Both MitM and MitN were overexpressed in E. coli using the pT7 promoter. Overexpression of both proteins was achieved by adding very low concentrations of IPTG (0.1 mM), and purification to homogeneity was achieved using a Ni-NTA column (FIG. 40).

Conversion of MA to MF by Both MitM and MitN

In vitro assay can not detect any MC conversion from the broth extract of MM107 by either MitM or MitN (data not shown), indicating that MitM is not the last step enzyme in MC biosynthesis. MA, MB, MC, MD and MH were employed as candidate substrates for both MitM and MitN. In the presence of SAM, a similar new product was formed with MA as substrate for both enzymes, while no conversions were detected with other mitomycins. The TLC and HPLC profiles of these new products were exactly the same as those of MF. To further locate the methyl group in the new compounds, the products were purified through HPLC and subjected to NMR analysis. In the ¹H NMR map, standard MA have three peaks for the three corresponding methyl groups: 1.83 (C6a methyl), 3.22 (C9a O-methyl), and 3.99 (C7a O-methyl). The extra N1a methyl group in MF adds a new peak at 2.74, as well as shifts the C9a O-methyl peak to 3.21. Both the MitM and MitN conversion products had the same four peaks (1.83, 2.74, 3.21, 3.99) as appeared in MF. The NMR result furier confirmed that the methyl group was added to N1a position of MA in both conversion products, and both MitM and MitN were N-methyltransferases.

Kinetic Data of the Conversions

In a range of pH 5.0-8.0, MitM has an optimal pH at 8.0, and the optimal pH for MitN is 7.4. Kinetic data were determined in pH 7.4 for both enzymes. As shown in Table 6, MitN had a higher (Km=94.7 μM) affinity to MA than MitM (Km=169.2 μM), while MitM had a higher (Km=22.8 μM) affinity to SAM than MitN (86.6 μM). However, MitN had a bigger Vmax value than MitM for either MA or SAM.

TABLE 6

| | | |
|---|---|---|
| Molecular Weight (kD) | 31 | 30 |
| Optimal pH | 8.0 | 7.4 |
| Km for MA [μM] | 169.2 +/−4.3 | 94.7 +/−4.5 |
| Vmax for MA (nmol . mg⁻¹ · min⁻¹) | 57.4 +/−4.2 | 75.9 +/−2.4 |
| Km for SAM [μM] | 22.8 +/−2.4 | 86.6 +/−4.5 |
| Vmax for SAM (nmol . mg⁻¹ · min⁻¹) | 29.8 +/−4.5 | 49.4 +/−5.3 |

Discussion

The discovery that both MitM and MitN are aziridine N-methyltransferases is unexpected. Based on the facts that disruption of mitM blocks MC production, while the mutant broth extract can not be converted into MC by MitM (data not shown), it can be concluded that MitM is a key but not the last step enzyme in MC biosynthesis. Taken together with the mitN disruption result, the functions of MitM and MitN in MC biosynthesis can be pictured as following:

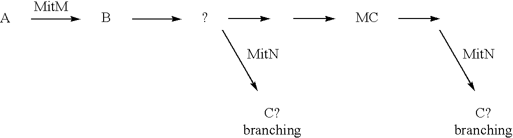

where A and B are the real substrate and, product of MitM, respectively. C is the product of MitN while the real substrate of MitN can be either the MC biosynthetic intermediates (branching) or downstream mitomycins after MC (tailoring).

As revealed by protein sequence analysis and in vitro conversion experiments, MitM is an N-methyltransferase. However, since there is no N-methyl group in MC molecule, the function of MitM in MC biosynthesis is questionable. One possibility is that the aziridine N-methylation serves as a temporary protection for other reactions and the methyl group will leave in the end. Another possibility is that in addition to its N-methyltransferase activity, MitM also has other functions, such as the C9a or C7 O-methyltransferase activities (even though it has no reaction with MB, MD and MH).

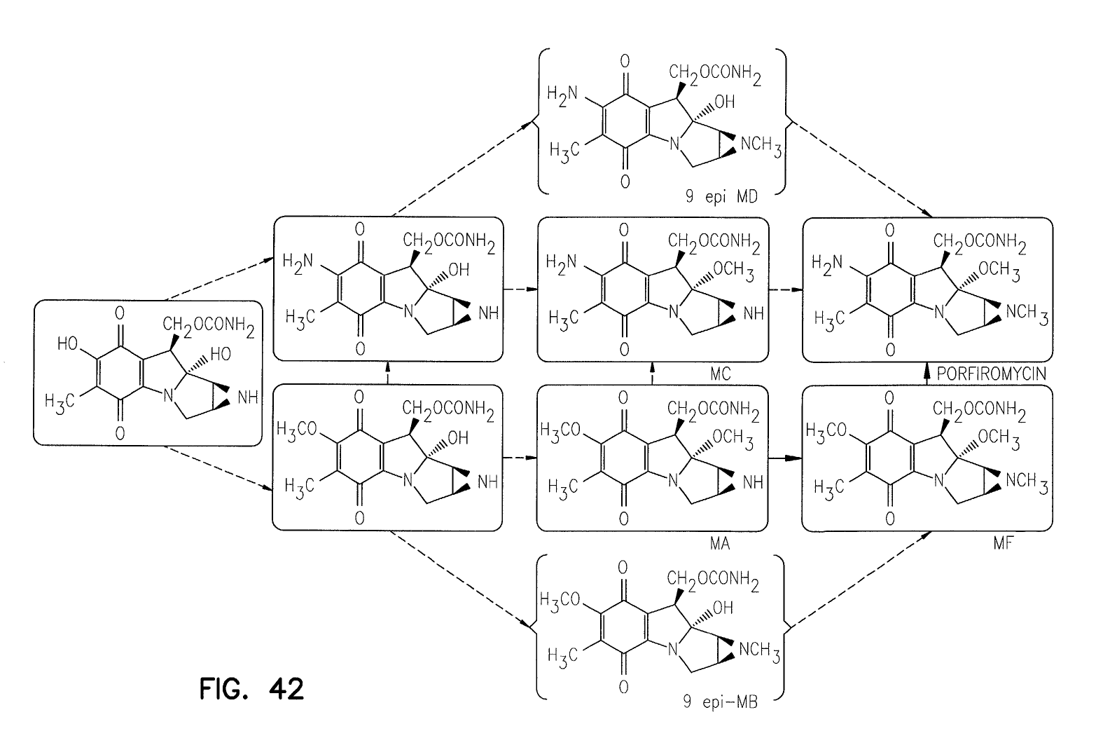

Considering the late stage mitomycin modifications, there are two possible routes concerning the order of transamination and methylation at C7 (FIG. 42). Hornemann (1981) had proposed that MA is the natural precursor of MC based on the following observations: 1) S. lavendulae which produces mainly MA and MB in some cultural conditions, produces MC exclusively under other conditions, indicating a close biosynthetic relationship between MA and MC (Wakaki et al., 1958); 2) MC can be obtained from MA simply by treatment of the latter with ammonia; and 3) a similar kind of transamination (from MA to MC) was detected in a feeding experiment of converting radio-labeled MF to porfiromycin (Hornemann, 1981).

Supporting this view, Tomohiro et al (1983) selected a S. lavendulae mutant strain which abolishes MC production but still accumulates MA and MB.

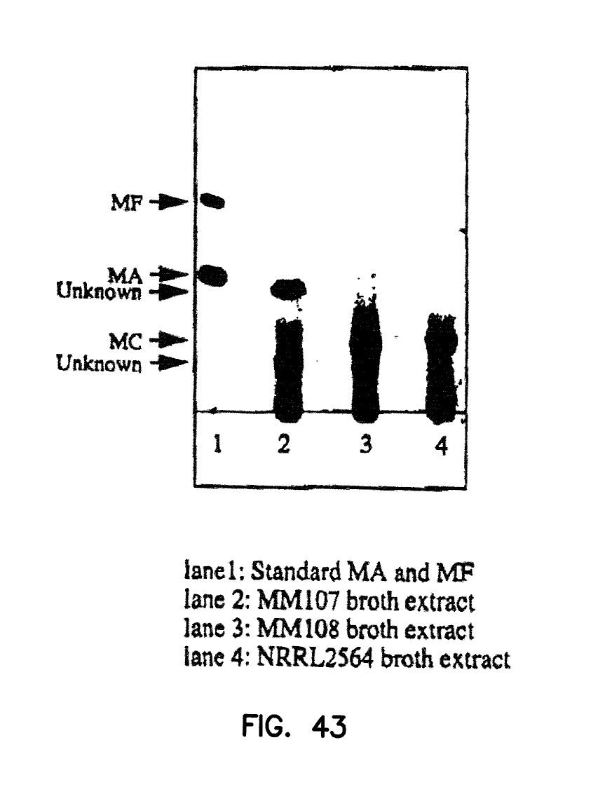

MM107 (mitM) blocks MA production while MM108 (mitN) may still produce MA (MA production was checked in both mutants. On TLC plates, the mitM mutant MM107 still produces three visible pink mitomycin compounds with one Rf value less than MC and the other two less than MA (FIG. 43)).

Both MitM and MitN have been confirmed to methylate MA but not MC, which indicates a certain level of substrate specificity. However, disruption and in vitro conversion results indicate that MA is probably not the real natural substrate for both MitM and MitN. Kinetic data revealed that both MitM and MitN had lower affinity to substrate but comparable affinity to SAM comparing to the purified Streptomyces antibiotic biosynthetic O-methyltransferases. These characterized enzymes include: macrocin O-methyltransferase (MOMT) and demethylmacrocin O-methyltransferase (DMOMT) for tylosin biosynthesis in S. fradiae (Bauer et al., 1988; Kreuzman et al., 1988), carminomycin O-methyltransferase (CMT) for daunomycin biosynthesis in S. sp. Strain C5 (Connors et al., 1993), 31-O-desmethylimmunomycin O-methyltransferase (DIMT) for immunomycin biosynthesis in S. hygroscopicus var ascomyceticus (Byrne et al., 1993), and 31-O-demethylFK506 O-methyltransferase (FKMT) for FK506 biosynthesis in S. sp. MA6858 (Shafiee et al., 1994). The Km values of MitM (169 μM) and MitN (95 μM) for MA are much higher than that of MOMT (5 μM), DMOMT (6 μM), CMT (0.5 μM), DIMT (11 μM), and FKMT (23 μM) for their corresponding substrates. For SAM, the Km of MitM (23 μM) is very close to the MOMT (23 μM), CMT (25 μM), DIMT (13 μM), and FKMT (28 μM) while the Km of MitN (87 μM) is comparable to DMOMT (111 μM).

The data described herein supports the hypothesis that both MitM and MitN are SAM-dependent methyltransferases for mitomycin biosynthesis. Nevertheless, the outcome that both of them are aziridine N-methyltransferases is unexpected. It is possible that MA, the hypothesized precursor for other mitomycins, is a favorable substrate for many a modification enzyme. Different expression level and kinetic balancing of such enzymes will in the end determine the diversity of mitomycin production. Thus, the results not only demonstrate aziridine N-methyltransferase activity but evidence the ability to engineer the biosynthesis of other mitomycin analogs.

In summary, two putative SAM dependent methyltransferases (mitM and mitN) previously assigned in the MC biosynthetic gene cluster were deleted in the MC producer S. lavendulae NRRL 2564. While the mitN deletion mutant MM108 slightly increases MC production, the mitM deletion mutant MM107 produces no MC but accumulates other bioactive intermediates. The disruption result indicates that MitM is a key enzyme for MC biosynthesis, whereas MitN probably serves as a branching or tailoring enzyme that leads to other mitomycins. Both MitM and MitN were overproduced in E. coli and purified to homogeneity. In the presence of SAM, both MitM and MitN can convert mitomycin A (MA) to mitomycin F (MF), but have no reactions with MB, MC, MD and MH. Kinetic analysis revealed that MitM had a higher affinity to SAM but lower affmity to MA than MitN. Thus, both MitM and MitN are novel mitomycin aziridine N-methyltransferases.

References

Alderson, G., D. A. Ritchie, C. Caballero, R. H. Cool, N. M. Ivanova, A. S. Huddleston, C. S. Flaxman, V. Kristufek, and A. Lounes. Physiology and genetics of antibiotic production and resistance. Res. Microbiol., 144, 665-672 (1993).

Altschul, S. F., Gish, W., Miller, W., Myers, E. W. and D. J. Lipman. Basic local alignment search tool, J. Mol. Biol., 215, 403-410 (1990).

Ames, G. Bacterial periplasmic transport systems: structure, mechanism, and evolution. Ann. Rev. Biochem., 55, 397-425 (1986).

Anderson, M. G., Kibby, J. J., Rickards, R. W. and J. M. Rothschild. Biosynthesis of the mitomycin antibiotics from 3-amino-5-hydroxybenzoic acid, J. Chem. Soc. Chem. Commun., 1277-1278 (1980).

Angell, S., Schwarz, A., and M. J. Bibb. The glucose kinase gene of Streptomyces coelicolor A3(2): its nucleotide sequence, transcriptional analysis and role in glucose repression, Mol. Microbiol., 6, 2833-2844 (1992).

August, P. R., Flickinger, M. C. and D. H. Sherman. Cloning and analysis of a locus (mcr) involved in mitomycin C resistance in Streptomyces lavendulae, J. Bacteriol., 176, 4448-4454 (1994).

August, P. R., Tang, L., Yoon, Y. J., Ning, S., Muller, R., Yu, T. W., Taylor, M., Hoffmann, D., Kim, C. G., Zhang, X., Hutchinson, C. R. and H. G. Floss. Biosynthesis of the ansamycin antibiotic rifamycin: deductions from the molecular analysis of the rif biosynthetic gene cluster of Amycolatopsis mediterranei S699, Chem. Biol., 5, 69-70 (1998).

Baltz, R. H. Genetic recombination in Streptomyces fradiae by protoplast fusion and cell regeneration, Dev. Ind. Microbiol., 21, 43-54 (1980).

Baltz, R. H., and T. J. Hosted. Molecular genetic methods for improving secondary-metabolite production in actinomycetes, Trends Biotech., 14:245-250 (1996).

Bauer, N. J., Kreuzman, A. J., Dotzlaf, J. E., and Yeh, W. K. Journal of Biological Chemistry, 263, 15619-25 (1988).

Beck, A., A. Kleickmann, M. Keller, W. Arnold, and A. Puhler. Identification and analysis of the Rhizobium meliloti exoAMONP genes involved in exopolysaccharide biosynthesis and mapping of promoters located on the exoHKLAMONP fragment, Mol. Gen. Genet., 241, 367-379 (1993).

Becker, A. M., Herlt, A. J., Hilton, G. L., Kibby, J. J. and R. W. Rickards. 3-Amino-5-hydroxybenzoic acid in antibiotic biosynthesis, VI. Directed biosynthesis studies with ansamycin antibiotics, J. Antibiot, 1323-1328 (1983).

Bennett, E. P., H. Hassan, and H. Clausen. cDNA cloning and expression of a novel human UDP-N-acetyl-alpha-D-galactosamine. Polypeptide N-acetylgalatosaminyltransferase, GalNAc-t3. J. Biol. Chem., 271, 17006-17012 (1996).

Berdy, J. Are actinomycetes exhausted as a source of secondary metabolites?, p. 13-14. In V. Debabov, Dudnik, Y. And Danlienko, V. (eds.), Ninth International Symposium on the Biology of Actinomycetes. All-Russia Scientific Research Institute for Genetics and Selection of Industrial Microorganisms, Moscow (1995).

Bezanson, G. S. and L. C. Vining. Studies on the biosynthesis of mitomycin C by Streptomyces verticillatus, Can. J. Biochem., 42, 911-918 (1971).

Bierman, M., Logan, R., O'Brien, K., Seno, E. T., Rao, R. N. and B. E. Schoner. Plasmid cloning vectors for the conjugal transfer of DNA from Escherichia coli to Streptomyces spp., Gene, 116, 43-49 (1992).

Blattner, F. R., Plunkett, G. R., Bloch, C. A., Perna, N. T., Burland, V., Riley, M., Collado-Vides, J., Glasner, J. D., Rode, C. K., Mayhew, G. F., Gregor, J., Davis, N. W., Kirkpatrick, H. A., Goeden, M. A, Rose, D. J., Mau, B. and Shao, Y., the complete genome sequence of Escherichia coli K-12, Science, 27, 1453-74 (1997).

Bouvier-Nave, P., Husselstein, T., Desprez, T. and Benveniste, P., Identification of cDNAs encoding sterol methyl-transferases involved in the second methylation step of plant sterol biosynthesis, Euro. J. Biochem., 246, 518-29 (1997).

Boyer, M. J., Bioreductive agents: a clinical update, Oncol. Res., 9, 391-395 (1997).

Brown, W. C., and J. L. Campbell. A new cloning vector and expression strategy for genes encoding proteins toxic to Escherichia coli, Gene, 127:99-103 (1993).

Bult, C. J., White, O., Olsen, G. J., Zhou, L., Flesichmann, R. D., Sutton, G. G., Blake, J. A., FitzGerald, L. M., Clayton, R. A., Gocayne, J. D., Kerlavage, A. R., Dougherty, B. A., Tomb, J. F., Adams, M. D., Reich, C. I., Overbeek, R., Kirkness, E. F., Weinstock, K. G., Merrick, J. M., Glodek, A., Scott, J. L., Geoghagen, N. and J. C. Venter. Complete genome sequence of the methanogenic archaeon, Methanococcus jannaschii, Science, 273, 1058-1073 (1996).

Butler, M. J., E. J. Friend, I. S. Hunter, F. S. Kaczmarek, D. A. Sugden, and M. Warren. Molecular cloning of resistance genes and architecture of a linked gene cluster involved in biosynthesis of oxytetracycline by Streptomyces rimosus. Mol. Gen. Genet., 215, 231-238 (1989).

Byrne, K. M., shafiee, A., Nielsen, J. B., Arison, B., Monaghan, R. C., and Kaplan, L. In: Developments in Industrial Microbiology Series (Nash, C., Hunter-Cevera, J., Cooper, R., Eveleigh, D. E., and Hamill, R., eds), pp. 29-47, Wn. C. Brown, Iowa (1993).

Caballero, J. L., Malpartida, F. and D. A. Hopwood. Transcriptional organization and regulation of an antibiotic export complex in the producing Streptomyces culture, Mol. Gen. Genet., 228, 372-380 (1991).

Chater, K. F. Genetic regulation of secondary metabolic pathways in Streptomyces. Ciba Foundation Symposium, 144-156 (1992).

Chater, K. F. and C. J. Bruton. Resistance, regulatory and production genes for the antibiotic methylenomycin are clustered, Embo Journal, 4, 1893-7 (1985).

Chiao, J. S., T. H. Xia, B. G. Mei, Z. K. Jin, and W. L. Gu. Rifamycin SV and related ansamycins, p. 477-498. In L. C. Vining and Stuttard, C. (Eds.), Genetics and biochemistry and antibiotic production. Butterworth-Heinemann, Newton, Mass. (1995).

Cole, S. T., Brosch, R., Parkhill, J., Garnier, T., Churcher, C., Harris, D., Gordon, S. V., Eiglmeier, K., Gas, S., Barry, C. R., Tekaia, F., Badcock, K., Basham, D., Brown, D., Chillingworth, T., Connor, R., Davies, R., Devlin, K., Feltwell, T., Gentles, S., Hamlin, N., Holroyd, S., Hornsby, T., Jagels, K., Barrell, B. G. and et al., Deciphering the biology of Mycobacterium tuberculosis from the complete genome sequence, Nature, 393, 537-544 (1998).

Connors, N. C., and Strohl, W. R. Journal of General Microbiology, 139, 1353-62 (1993).

Coque, J., P. Liras, and J. Martin. Genes for a β-lactamase, a penicillin-binding protein and a transmembrane protein are clustered with the cephamycin biosynthetic genes in Nocardia lactamdurans. EMBO. J. 12, 631-639 (1993).

Coque, J. J., Perez-Laraine, F. J., Enguita, F. J., Fuente, J. L., Martin, J. F. and P. Liras. Characterization of the cmcH genes of Nocardia lactamdurans and Streptomyces clavuligerus encoding a functional 3′-hydroxymethylcephem O-carbamoyltransferase for cephamycin biosynthesis, Gene, 162, 21-27 (1995).

Cummings, J., Spanswick, V. J., Tomasz, M. and J. F. Smyth. Enzymology of mitomycin C metabolic activation in tumor tissue—implications for enzyme-directed bioreductive drug development, Biochemical Pharmacology, 56, 405-414 (1998).

Cundliffe, E. Self-protection mechanisms in antibiotic producers. Ciba Found. Symp., 171, 199-208 (1992).

Cundliffe, E., L. A. Merson-Davies, and G. H. Keleman. Aspects of tylosin production and resistance in Streptomyces fradiae, p. 235-243, Industrial microorganisms: basic and applied molecular genetics. American Society for Microbiology, Washington, D.C. (1993).

Dean, C. R., Neshat, S. and K. Poole. PfeR, an enterobactin-responsive activator of ferric enterobactin receptor gene expression in Pseudomonas aeruginosa, J. Bacteriol., 178, 5361-5369 (1996).

Decker, H., Motamedi, H. and C. R. Hutchinson. Nucleotide sequences and heterologous expression of tcmG and tcmP, biosynthetic genes for tetracenomycin C in Streptomyces glaucescens, J. Bacteriol., 175, 3876-3886 (1993).

Denis, F. and R. Brzezinski. A versatile shuttle cosmid vector for use in Escherichia coli and Actinomycetes, Gene, 111, 115-118 (1992).

Deppenmeier, U., Muller, V. and G. Gottschalk. Pathways of energy conservation in methanogenic archaea, Arch. Microbiol., 165, 149-163 (1996).

Devereux, J., Haeberli, P. and O. Smithies. A comprehensive set of sequence analysis programs for the VAX, Nucleic Acids Res., 12, 387-395 (1984).

Dewick, P. M., The biosynthesis of shikimate metabolites, Nat. Prod. Rep., 15, 17-58 (1995).

Dickens, M. L., Ye, J. and W. R. Strohl. Analysis of clustered genes encoding both early and late steps in daunomycin biosynthesis by Streptomyces sp. strain C5. J. Bacteriol., 177, 536-543 (1995).

Dittrich, H. and T. M. Kutchan. Molecular cloning, expression, and induction of berberine bridge enzyme, an enzyme essential to the formation of benzophenanthridine alkaloids in the response of plants to pathogenic attach, Proc. Natl., Acad., Sci. USA, 88, 9969-9973 (1991).

Donadio, S., M. J. Staver, J. B. McAlpine, S. J. Swanson, and L. Katz. Modular organization of genes required for complex polyketide biosynthesis. Science, 252, 675-679 (1991).

Evans, J. W., Yudoh, K., Delahoussaye, Y. M. and J. M. Brown. Tirpazamine is metabolized to its DNA-damaging radical by intranuclear enzymes, Cancer Research, 58, 2098-2101 (1998).

Fernandez-Moreno, M. A., Caballero, J. L., Hopwood, D. A. and F. Malpartida. The act cluster contains regulatory and antibiotic export genes, direct targets for translational control by the bldA tRNA gene of Streptomyces, Cell, 66, 769-80 (1991).

Floss, H. G. Natural products derived from unusual variants of the shikimate pathway, Nat. Prod. Rep., 14, 433-52 (1997).

Gandecha, A. R., Large, S. L., and Cundliffe, E. Gene, 184, 197-203 (1997).

Ghisalba, O., and N. Nuesch. A genetic approach to the biosynthesis of the rifamycin-chromophore in Nocardia mediterraniae. IV. Identification of 3-amino-5-hydroxybenzoic acid as a direct precursor of the seven-carbon amino starter-unit. J. Antibiot., 34, 64-71 (1981).

Gibson, J., Dispensa, M., Fogg, G. C., Evans, D. T. and C. S. Harwood. 4-Hydroxybenzoate-coenzyme A ligase from Rhodopseudomonas palustris: purification, gene sequence, and role in anaerobic degradation, J. Bacteriol., 176, 634-641 (1994).

Grebenok, R. J., Galbraith, D. W. and D. D. Penna. Characterization of Zea mays endosperm C-24 sterol methyltransferase: one of two types of sterol methyltransferase in higher plants, Plant Mol. Biol., 34, 891-6 (1997).

Grkovic, S., Brown, M. H., Roberts, N. J., Paulsen, I. T. and R. A. Skurray. QacR is a repressor protein that regulates expression of the Staphylococcus aureus multidrug efflux pump QacA, J. Biol. Chem., 273, 18665-73 (1998).

Guilfoile, P. G. and C. R. Hutchinson. Sequence and transcriptional analysis of the Streptomyces glaucescens tcmAR tetracenomycin C resistance and repressor gene loci, Journal of Bacteriology, 174, 3651-3658 (1992).

Guilfoile, P. G. and C. R. Hutchinson. The Streptomyces glaucescens TcmR protein represses transcription of the divergently oriented tcmR and tcmA genes by binding to an intergenic operator region, Journal of Bacteriology, 174, 3659-66 (1992).

Hara, O., and Hutchinson, C. R. Journal of Bacteriology, 174, 5141-4 (1992).

Hardwick, K. G. and H. R. Pelham. SED6 is identical to ERG6, and encodes a putative methyltansferase required for ergosterol synthesis, Yeast, 10, 265-269 (1994).

Hata, T., Sano, Y., Sugawara, R., Matsumae, A., Kanamori, K., Shima, T. and T. Hoshi. Mitomycin, a new antibiotic from Streptomyces, J. Antibiot. Ser. A, 9, 141-146 (1956).

Hatano, K., S. Akiyama, M. Asai, and R. W. Richards. Biosynthetic origin of amino benzenoid nucleus (C₇N-unit) of ansamitocin, a group of novel maytansinoid antibiotics. J. Antibiot., 35, 1415-1417 (1982).

Haydock, S. F., Dowson, J. A., Dhillon, N., Roberts, G. A., Cortes, J. and P. F. Leadlay. Cloning and sequence analysis of genes involved in erythromycin biosynthesis in Saccharopolyspora erythraea: sequence similarities between EryG and a family of S-adenosylmethionine-dependent methyltransferases, Mol. Gen. Genet., 230, 120-128 (1991).

Henderson, I. C., Recent Advances in the Usage of Mitomycin, Proceedings of a symposium, Hawaii, March 21-24, Oncology, 1, 1-83 (1993).

Henderson, C. I., Recent advances in the usage of mitomycin, Oncology, 50:(Suppl. 1), 1-84 (1993).

Hidaka, T., Goda, M., Kuzuyama, T., Takei, N., Hidaka, M. and H. Seto. Cloning and nucleotide sequence of fosfomycin biosynthetic genes of Streptomyces wedmorensis, Mol. Gen., Genet., 249, 274-280 (1995).

Hidaka, T., Hidaka, M., Kuzuyama, T. and H. Seto. Sequence of a P-methyltransferase-encoding gene isolated from a bialaphos-producing Streptomyces hygroscopicus, Gene, 158, 149-150 (1995).

Higgins, C., S. Hyde, M. Mimmack, U. Gileadi, D. Gill, and M. Gallagher. Binding protein-dependent transport systems. J. Bioenerg., Biomem., 22, 571-592 (1990).

Hirai, O., Miyamae, Y., Hattori, Y., Takashima, M., Miyamoto, A., Zaizen, K. and Y. Mine. Microbial mutagenicity an in vitro chromosome aberration induction by fk973, a new antitumor agent, Mutation Res., 324, 43-50 (1994).

Hopwood, D. A. Genetic contributions to understanding polyketide synthases. Chem. Rev., 97,2465-2497 (1997).

Hopwood, D. A., Bibb, M. J., Chater, K. F., Kieser, T., Bruton, C. J., Kieser, H. M., Lydiate, D. J., Smith, C. P., Ward, J. M. and H. S. Schrempf. Genetic manipulation of Streptomyces: a laboratory manual, John Innes Institute, Norwich, United Kingdom, 1985.

Horii, M., Ishizaki, T., Paik, S. Y., Manome, T. and Y. Murooka. An operon containing the genes for cholesterol oxidase and a cytochrome P-450-like protein from a Streptomyces sp., J. Bacteriol., 12, 3644-3653 (1990).

Hornemann, U., Biosynthesis of the mitomycins, 1981.

Hornemann, U. and M. J. Heins. Stereochemical relationship between mitomycins A, B, and C. J. Org. Chem., 50, 1301-1302 (1985).

Hornemann, U. and J. H. Eggert. Utilization of the intact carbamoyl group of L-(NH₂CO—¹³C, ¹⁵N) citrulline in mitomycin biosynthesis by Streptomyces verticillatus, Journal of Antibiotics, 28, 841-843 (1975).

Hornemann, Y., Kehrer, J. P., Nunez, C. S. and R. L. Ranieri. D-glucosamine and L-citrulline, precursors in mitomycin biosynthesis by Streptomyces verticillatus, Journal of the American Chemical Society, 96, 320-322 (1974).

Ikeda, H., Nonomiya, T., Usami, M., Ohta, T., and Omura, S. Proceedings of the National Academy of Sciences of the United States of America, 96, 9509-14 (1999).

Iyer, N., and W. Szybalski. Mitomycin or porfiromycin: chemical mechanism of activation and cross-linking of DNA. Science, 145, 55-56 (1964).

Jabbouri, S., Fellay, R., Talmont, F., Kamalaprija, P., Burger, U., Relic, B., Prome, J. C. and W. J. Broughton. Involvement of nodS in N-methylation and nodU in 6-O-carbamoylation of Rhizobium sp. NGR234 nod factors, J. Biol. Chem., 270, 22968-22073 (1995).

Jabbouri, S., Relic, B., Hanin, M., Kamalaprija, P., Burger, U., Prome, D., Prome, J. C. and W. J. Broughton. nolO and noeI (HsnII) of Rhizobium sp. NGR234 are involved in 3-O-carbamoylation and 2-O-methylation of Nod factors, J. Biol. Chem., 273, 12047-12055 (1998).

Johnson, D. A., August, P. R., Shackleton, C., Liu, H. W. and D. H. Sherman. Microbial resistance to mitomycins involves a redox relay mechanism, J. Am. Chem. Soc., 119, 2576-2577 (1997).

Kagan, R. M. and S. Clarke. Widespread occurrence of three sequence motifs in diverse S-adenosyhnethionine-dependent methyltransfemases suggests a common structure for these enzymes, Arch. Biochem. Biophy., 310, 417-27 (1994).

Kahler, C. M., R. W. Carlson, M. M. Rahman, L. E. Martin, D. S. Stephens. Two glycosyltransferase genes, IgtF and rfak, constitute the lipooligosaccharide ice (inner core extension) biosynthesis operon of Neisseria meningitidis. J. Bacteriol., 178, 6677-6684 (1996).

Kasai, M. and H. Arai. Novel mitomycin derivatives, Exp. Opin. Ther. Patents, 5, 757-770 (1995).

Kibby, J. J., I. A. McDonald, and R. W. Rickards. 3-amino-5-hydroxybenzoic acid as a key intermediate in ansamycin and maytansinoid biosynthesis. J. Chem. Soc. Chem. Comm., 198, 768-769 (1980).

Kibby, J. J. and R. W. Rickards. The identification of 3-amino-5-hydroxybenzoic acid as a new natural aromatic amino acid, J. Antibiot., 34, 605-607 (1981).

Kim, C. G., Kirschning, A., Bergon, P., Zhou, P., Su, E., Sauerbrei, B., Ning, S., Ahn, Y., Breuer, M., Leistner, E. and H. G. Floss. Biosynthesis of 3-amino-5-hydroxybenzoic acid, the precursor of mC₇N units in ansamycin antibiotics, J. Am. Chem., Soc., 188, 7486-7491 (1996).

Kim, C. G., A. Kirschning, P. Bergon, Y. Ahn, J. J. Wang, M. Shibuya, and H. G. Floss. Formation of 3-amino-5-hydroxybenzoic acid, the precursor of mC₇N units in ansamycin antibiotics, by a new variant of the shikimate pathway. J. Am. Chem. Soc., 114, 4941-4943 (1992).

Kim, C. G., Yu, T. W., Fryhle, C. B., Handa, S. and H. G. Floss. 3-Amino-5-hydroxybenzoic acid synthase, the terminal enzyme in the formation of the precursor of mC₇N units in rifamycin and related antibiotics, J. Biol. Chem., 273, 6030-6040 (1998).

Kirsch, E. J., and Korshalla, J. D. J. Bacteriol., 87, 247-255 (1964).

Kreuzman, A. J., Turner, J. R., and Yeh, W. K. Journal of Biological Chemistry, 263, 15626-33 (1988).

Kunz, K. R., Iyengar, B. S., Dorr, R. T., Alberts, D. S., and W. Remers. W. A. Structure-activity relationships for mitomycin C and mitomycin analogues. J. Med. Chem., 3, 2281-2286 (1991).

Kuzuyama, T., Seki, T., Dairi, T., Hidaka, T. and H. Seto. Nucleotide sequence of fortimicin KL1 methyltransferase gene isolated from Micromonospora olivasterospora and comparison of its deduced amino acid sequence with those of methyltransferases involved in the biosynthesis of bialaphos and fosfomycin, J. Antibiot., 48, 1191-3 (1995).

Kwon, O., Bhattacharyya, D. K. and R. Meganathan. Menaquinone (vitamin K2) biosynthesis: overexpression, purification, and properties of o-succinylbenzoyl-coenzyme A synthetase from Escherichia coli, J. Bacteriol., 178, 6778-6781 (1996).

Kyte, J., and R. F. Doolittle. A simple method for displaying the hydropathic character of a protein. J. Mol. Biol., 157, 105-132 (1982).

Lacalle, R. A., Ruiz, D. and A. Jimenez. Molecular analysis of the dmpM gene encoding an O-dimethyl puromycm O-methyltransferase from Streptomyces alboniger, Gene, 109, 55-61 (1991)

Lee, J. P., S. W. Tsao, C. J. Chang, X. G. He, and H. G. Floss. Biosynthesis of naphthomycin A in Streptomyces collinus. Can. J. Chem., 72, 182-187 (1994).

Lee, P. J. and A. M. Stock. Characterization of the genes and proteins of a two-component system from the hyperthermophilic bacterium Thermotoga maritima, J. Bacteriol., 178, 5579-5585 (1996).

Levy, S., and L. McMuwry. Plasmid-mediated tetracycline resistance involves alternative transport systems for tetracycline. Nature, 226, 90-92 (1978).

Littlejohn, T., I. Paulsen, M. Gillespie, J. Tennant, M. Midgley, I. Jones, A. Purewal, and R. Skurray. Substrate specificity and energetics of antiseptic and disinfectant resistance in Staphylococcus aureus. FEMS Microbiol. Lett., 95, 259-266 (1992).

Lomovskaya, O., Lewis, K. and A. Matin. EmrR is a negative regulator of the Escherichia coli multidrug resistance pump EmrAB, J. Bacteriol., 177, 2328-2334 (1995).

Luka, S., Sanjuan, J., Carlson, R. W. and G. Stacey. nolMNO genes of Bradyrhizobium japonicum are co-transcribed with nodYABCSUIJ, and nolO is involved in the synthesis of the lipo-oligosaccharide nodulation signals, J. Biol. Chem., 268, 27053-27059 (1993).

Lundstrom, K., Tenhunen, J., Tilgmann, C., Karhunen, T., Panula, P., and Ulmanen, I. Biochimica et Biophysica Acta, 1251, 1-10 (1995).

Madduri, K., Torti, F., Colombo, A. L. and C. R. Hutchinson. Cloning and sequencing of a gene encoding carminomycin 4-O-methyltransferase from Streptomyces peucetius and its expression in Escherichia coli, J. Bacteriol., 175, 3900-3904 (1993).

Makino, K., Shinagawa, H., Amemura, M. and A. Nakata. Nucleotide sequence of the phoB gene, the positive regulatory gene for the phosphate regulon of Escherichia coli K-12, J. Mol. Biol., 190, 37-44 (1986).

Mao, Y., Varoglu, M., and D. H. Sherman. Molecular characterization and analysis of the biosynthetic gene cluster for the antitumor antibiotic mitomycin C from Streptomyces lavendulae NRRL 2564. Chem. Biol., 4, 251-263 (1999a).

Mao, Y., Varoglu, M., and D. H. Sherman. Genetic localization and molecular characterization of two key genes (mitAB) required for biosynthesis of the antitumor antibiotic mitomycin C. J. Bacteriol., 181, 2199-2208 (1999b).

Martin, J. F. Clusters of genes for the biosynthesis of antibiotics: regulatory genes and overproduction of pharmaceuticals. J. Ind. Microbiol., 9, 73-90 (1992).

Mazodier, P., Petter, R. and C. Thomson. Intergeneric conjugation between Escherichia coli and Streptomyces species, J. Bacteriol., 171, 3583-3585 (1989).

Mendez, C., and J. A. Salas. ABC transporters in antibiotic-producing actinomycetes. FEMS Microb. Lett., 158, 1-8 (1998).

Miller, J., J. Olson, J. Plfugrath, and F. Quiocho. Rates of ligand binding to periplasmic proteins involved in bacterial transport and chemotaxis. J. Biol Chem., 238, 13665-13672 (1983).

Mizuno, T. and I. Tanaka. Structure of the DNA-binding domain of the OmpR family of response regulators, Mol. Microbiol., 24, 665-667 (1997).

Morbidoni, H. R., de Mondoza, D. and J. Cronan Jr. Bacillus subtilis acyl carrier protein is encoded in a cluster of lipid biosynthesis genes, J. Bacteriol., 178, 4794-800 (1996).

Motamedi, H., and C. R. Hutchinson. Cloning and heterologous expression of a gene cluster for the biosynthesis of tetracenomycin C, the anthracycline antitumor antibiotic of Streptomyces glaucescens. Proc. Natl. Acad. Sci. USA, 84, 4445-4449 (1987).

Motamedi, H., Shafiee, A., Cai, S. J., Streicher, S. L., Arison, B. H., and Miller, R. R. Journal of Bacteriology, 178, 5243-8 (1996).

Muth, G., B. Nussbaumer, W. Wohlleben, and A. Publer. A vector system with temperature-sensitive replication for gene disruption and mutational cloning in streptomycetes. Mol. Gen. Genet., 219, 341-348 (1989).

Naoe, Y., Inami, M., Matsumoto, S., Nishigaki, F., Tsujimoto, S., Kawamura, I., Miyayasu, K., Manda, T. and K. Shimomura. Fk317—a novel substituted dihydrobenzoxazine with potent antitumor activity which does not induce vascular leak syndrome, Cancer Chemo. Pharmacol., 42, 31-36 (1998).

Neal, R. J., and K. F. Chater. Nucleotide sequence analysis reveals similarities between proteins determining methylenomycin A resistance in Streptomyces and tetracycline resistance in eubacteria. Gene, 5, 229-241 (1987).

Niemi, J. and Mantsala, P., Nucleotide sequences and expression of genes from Streptomyces purpurascens that cause the production of new anthracyclines in Streptomyces galilaeus, J. Bacteriol., 177, 2942-2945 (1995).

Nikaido, H. Prevention of drug access to bacterial targets: Permeability barriers and active efflux. Science, 264, 382-388 (1994).

Nishikohri, K. and S, Fukui. Biosynthesis of mitomycin in Streptomyces caespitosus. Relationship of intracellular vitamin B₁₂ level to mitomycin synthesis and enzymatic methylation of a demethylated derivative of mitomycin, Eur. J. Appl. Microbiol., 2, 129-145 (1975).

Nolling, J., Pihl, T. D. and J. N. Reeve. Cloning, sequencing, and growth phase-dependent transcription of the coenzyme F420-dependent N5, N10-methylenetetrahydromethanopterin reductase-encoding genes from Methanobacterium thermoautotrophicum delta H and Methanopyrus kandleri, J. Bacteriol., 177, 7238-7244 (1995).

Ogawa, H., Gomi, T., Takusagawa, F., and Fujioka, M. International Journal of Biochemistry & Cell Biology, 30, 13-26 (1998).

Okinaga, S., Ohrui, T., Nakazawa, H., Yamauchi, K., Sakurai, E., Watanabe, T., Sekizawa, K., and Sasaki, H. Methods & Findings in Experimental & Clinical Pharmacology. 17, 16-20 (1995).

Ohno, T., Armand, S., Hata, T., Nikaidou, N., Henrissat, B., Mitsutomi, M. and T. Watanabe. A modular family 19 chitinase found in the prokaryotic organism Streptomyces griseus HUT 6037, J. Bacteriol., 178, 5065-5070 (1996).

Omer, C. A., Lenstra, R., Little, P. J., Dean, C., Tepperman, J. M., Leto, K. J., Romesser, J. A. and D. P. O'Keefe. Genes for two herbicide-inducible cytochromes P-450 from Streptomyces griseolus, J. Bacteriol., 172, 3335-3345 (1990).

Otten, S. L., X. Liu, J. Ferguson, and C. R. Hutchinson. Cloning and characterization of the Streptomyces peucetius dnrQS genes encoding a daunosamine biosynthesis enzyme and a glycosyl transferase involved in daunorubicin biosynthesis. J. Bacteriol., 177, 6688-6692 (1995b).

Otten, S. L., Ferguson, J. and C. R. Hutchinson. Regulation of daunorubicin production in Streptomyces peucetius by the dnrR2 locus, J. Bacteriol., 177, 1216-1224 (1995a).

Pan, S. S. and T. Iracki. Metabolites and DNA adduct formation from flavoenzymne-activated portfromycin, Molecular Pharmacology, 34, 223-228 (1988).

Paulsen, I., and R. Skurray. Topology, structure and evolution of two families of proteins involved in antibiotic and antiseptic resistance in eukaryotes and prokaryotes—an analysis. Gene, 124:1-11 (1993).

Paulsen, I., M. Brown, and R. Skurray. Proton-dependent multidrug efflux pumps. Microbiol. Rev., 60, 575-608 (1996).

Paz, M. M. and P. B. Hopkins. DNA-DNA interstrand cross-linking by FR66979-intermediates in the activation cascade, J. Am. Chem. Soc., 119, 5999-6005 (1997).

Perez-Laraine, F. J., Liras, P., Rodriguez-Garcia, A. and J. F. Martin. A regulatory gene (ccaR) required for cephamycin and clavulanic acid production in Streptomyces clavuligenas: amplification results in overproduction of both beta-lactam compounds, J. Bacteriol., 179, 2053-2059 (1997).

Peschke, U., H. Schmidt, H. Z. Zhang, and W. Piepersberg. Molecular characterization of the lincomycin-production gene cluster of Streptomyces lincolnensis. 78-11. Mol. Microbiol., 16, 1137-1156 (1995).

Piepersberg, W. Pathway engineering in secondary metabolite-producing actinomycetes, Crit. Rev. Biotechnol., 14:251-285 (1994).

Platt, M. W., Miller, K. J., Lane, W. S. and E. P. Kennedy. Isolation and characterization of the constitutive acyl carrier protein from Rhozobium meliloti, J. Bacteriol., 172, 5440-4 (1990).

Potgieter, M. Biosynthetic studies on geldanamycin and pactamycin. Ph.D. thesis. Univ. Illinois (1983).

Praillet, T., Nasser, W., Robert-Baudouy, J. and S. Reverchon. Purification and functional characterization of Pacs, a regulator of virulence-factor synthesis in Erwinia chrysanthemi, Molecular Microbiology, 20, 391-402 (1996).

Rodriguez, A. M., Olano, C., Mendez, C., Hutchinson, C. R. and J. A. Salas. A cytochrome P450-like gene possibly involved in oleandomycin biosynthesis by Streptomyces antibioticus, FEMS Microbiol. Lett., 127, 117-20 (1995).

Rouch, D., D. Cram, D. DiBerardino, T. Littlejohn and R. Skurray. Efflux-mediated antiseptic resistance gene qacA from Staphylococcus aureus: common ancestry with tetracycline and sugar-transport proteins. Mol. Microbiol., 4, 2051-2062 (1990).

Sambrook, J., Fritsch, E. F. and Maniatis, T., Molecular cloning a laboratoy manual, 2nd ed, Cold Spring Harbor Laboratory Press, Cold Spring Harbor, N.Y. (1989).

Sartorelli, A. C., W. F. Hodnick, M. F. Belcourt, M. Tomasz, B. Haffty, J. J. Fischer, and S. Rockwell. Mitomycin C: a prototype bioreductive agent, Oncol. Rec., 6:501-508 (1994).

Schaferjohann, J., Yoo, J. G., Kusian, B. and B. Bowien. The cbb operons of the facultative chemoautotroph Alcaligenes eutrophus encode phosphoglycolate phosphatase, J. Bacteriol., 175, 7329-40 (1993).

Schwecke, T., Aparicio, J. F., Molnar, I., Konig, A., Khaw, L. E., Haydock, S. F., Oliynyk, M., Caffrey, P., Cortes, J., Lester, J. B. and et al., The biosynthetic gene cluster for the polyketide immunosuppressant rapamycin, Proc. Natl. Acad. Sci. USA, 92, 7839-43 (1995).

Seno, E. T. and R. H. Baltz. Structural organization and regulation of antibiotic biosynthesis and resistance genes in actino ycetes, CRC Press, Boca Raton, Fla. (1989).

Shafiee, A., Motamedi, H., and Chen, T. European Journal of Biochemistry 225, 755-64 (1994).

Sheldon, P. J., Johnson, D. A., August, P. J., Liu, H. W. and D. H. Sherman. Characterization of a mitomycin-binding drug resistance mechanism from the producing organism, Streptomyces lavendulae, J. Bacteriol., 172, 1796-1804 (1997).

Shi, J., Gonzales, R. A. and Bhattacharyya, M. K., Identification and characterization of an S-adenosyl-L-methionine: delta 24-sterol-C-methyltransferase cDNA from soybean, J. Biol. Chem., 211, 9384-9389 (1996).

Shikano, M., Onimura, K., Fukai, Y., Hori, M., Fukazawa, H., Mizuno, S., Yazawa, K. and Y. Uehara 1a-docosahexaenoyl mitomycin C: a novel inhibitor of protein tyrosine kinase, Biochem. Biophys. Res. Commun., 248, 858-863 (1998).

Shirahata, K., Morimota, M., Ashizawa, T., Mineura, K., Kono, M., Saito, Y., and Kasai, M. In: Program and abstracts of the 21st Intersci. Conf. on Antimicrob. Agents Chemother., 421, pp. 4-6, Chicago (1981).

Simon, R., U. Priefer, and A. Puhler, A broad host range mobilization system for in vivo genetic engineering: Transposon mutagenesis in Gram negative bacteria, Bio/Technology, 1:784-791 (1983).

Smith, T. M., Y. F. Jiang, P. Shipley, and H. G. Floss. The thiostrepton-resistance encoding gene in Streptomyces laurentii is located within a cluster of ribosomal protein operons. Gene, 164, 137-142 (1995).

Smitskampwilms, E., Hendriks, H. R. and Peters, G. J., Development, pharmacology, role of DT-diaphorase and prospects of the indoloquinone EO9, Gen. Pharmacol., 27, 421-429 (1996).

Solenberg, P. J., P. Matsushima, D. R. Stack, S. C. Wilkie, R. C. Thompson, and R. H. Baltz. Production of hybrid glycopeptide antibiotics in vitro and in Streptomyces toyocaensis. Chem. Biol., 4, 195-202 (1997).

Spanswick, V. J., Cummings, J. and J. F. Smyth. Current issues in the enzymology of mitomycin C metabolic activation, Gen. Pharmacol., 31, 539-544 (1998).

Spath, C., Kraus, A. and W. Hillen. Contribution of glucose kinase to glucose repression of xylose utilization in Bacillus megaterium, J. Bacteriol., 179, 7603-7605 (1997).

Stackebrandt, E., and C. R. Woese. Towards a phylogeny of the actinomycetes and related organisms. Curr. Microbiol., 5, 197-202 (1981).

Staley, A. L., and K. L. Rinehart. Biosynthesis of the streptovaricins: 3-amino-5-hydroxybenzoic acid as a precursor to the meta-C₇N unit. J. Antibiot., 44, 218-224 (1991).

Stupperich E., Juza, A., Hoppert, M. and F. Mayer. Cloning, sequencing and immunological characterization of the corrinoid-containing subunit of the N5-methyltetrahydromethanopterin: coenzyme-M methyltransferase from Methanobacterium thermoautotrophicum, Euro. J Biochem., 217, 115-121 (1993).

Summers, R. G., Wendt-Pienkowski, E., Motamedi, H. and C. R. Hutchinson. Nucleotide sequence of the tcmII-tcmIV region of the tetracenomycin C biosynthetic gene cluster of Streptomyces glaucescens and evidence that the tcmN gene encodes a multifunctional cyclase-dehydratase-O-methyl transferase, J. Bacteriol., 174, 1810-1820 (1992).

Takano, E., Gramajo, H. C., Strauch, E., Andres, N., White, J. and M. J. Bibb. Transcriptional regulation of the redD transcrptional activator gene accounts for growth-phase-dependent production of the antibiotic undecylprodigiosin in Streptomyces coelicolor A3(2), Molecular Microbiology, 6, 2797-2804 (1992).

Tang, L., Grimm, A., Zhang, Y. X. and C. R. Hutchinson. Purification and characterization of the DNA-binding protein DnrI, a transcriptional factor of daunorubicin biosynthesis in Streptomyces peucetius, Molecular Microbiology, 22, 801-13 (1996).

Tercero, J., R. Lacalle, and A. Jimenez. The pur8 gene from the pur cluster of Streptomyces alboniger encodes a highly hydrophobic polypeptide which confers resistance to puromycin. Eur. J. Biochem., 218, 963-971 (1993).

Tercero, J. A., Espinosa, J. C., Lacalle, R. A., and Jimenez, A. J. Biol. Chem., 271, 1579-1590 (1996).

Thauer, R. K, Hedderich, R. and R. Fischer. Reactions and enzymes involved in methanogenesis from CO₂ and H₂, Chapman and Hall, New York, N.Y., 1993.

Tomasz, M. Mitomycin C: small fast and deadly (but very selective), Chemistry and Biology, 2, 575-579 (1995).

Tomasz, M. and Y. Palom. The mitomycin bioreductive antitumor agents: cross-linking and alkylation of DNA as the molecular basis of their activity, Pharmacol. Therap., 76, 73-87 (1997).

Tomohiro, S., Arai, Y., and Mineura, K.In: Eur. Pat. Appl., pp. 74245 (1983).

Tuan, J. S., Weber, J. M., Staver, M. J., Leung, J. O., Donadio, S. and L. Katz. Cloning of the genes involved in erythromycin biosynthesis from Saccaropolyspora erythraea using a novel Actinomycete-Eschelichia coli cosmid, Gene, 90, 21-29 (1990).

Turgay, K., and M. A. Marahiel. A general approach for identifying and cloning peptide synthetase genes. Peptide Res., 7, 238-241 (1994).

Vance, D. E., and Walkey, C. J. Current Opinion In Lipidology, 9, 125-30 (1998).

Vara, J., F. Malpartida, D. A. Hopwood, and A. Jimenez. Cloning and expression of a puromycin N-acetyl transferase gene from Streptomyces alboniger in Streptomyces lividans and Escherichia coli Gene 33, 197-206 (1985).

Vaupel, M. and R. K. Thauer. Coenzyme F420dependent N5,N10-methylenetetahydromethanopterin reductase (Mer) from Methanobacterium thermoautotrophicum strain Marburg. Cloning, sequencing, transcriptional analysis, and functional expression in Escherichia coli of the mer gene, Euro. J. Biochem, 231, 773-8 (1995).

Verweij, J. Mitomycins, Cancer Chemotherapy and Biological Response Modifiers, 17, 46-58 (1997).

Wakaki, K., Harumo, H., Tomioka, K., Shimizu, G., Kato, E., Kamada, H., Kudo, S. and Y. Fujimoto. Isolation of new fractions of antitumor mitomycins, Antibiot. Chemother., 8, 228-240 (1958).

Webb, J. S., D. B. Cosalich, T. H. Mowat, J. B. Patrick, R. W. Broschard, W. E. Meyor, R. P. Williams, C. F. Wolf, W. Fulmore, C. Pidacks, and J. E. Lancaster. The structure of Mitomycins A, B, and C and Porfiromycin-Part 1. J. Am. Chem. Soc., 84, 3185-3188 (1962).

Weinshilboum, R. M., Otterness, D. M., and Szumlanski, C. L. Annu. Rev. Pharmacol. Toxicol., 39, 19-52 (1999).

White, P. J., Young, J., Hunter, I. S., Nimmo, H. G. and J. R. Coggins. The purification and characterization of 3-dehydroquinase from Streptomyces coelicolor, Biochem., J., 265, 735-8 (1990).

Wietzorrek, A. and M. Bibb. A novel family of proteins that regulates antibiotic production in streptomycetes appears to contain an OmpR-like DNA-binding fold, Molecular Microbiology, 25, 1181-4 (1997).

Williams, R. M., Rajski, S. R. and S. B. Rollins. FR900482, a close cousin of mitomycin C that exploits mitosene-based DNA cross-linking, Chemistry and Biology, 4, 127-137 (1997).

Wu, T. S., J. Duncan, S. W. Tsao, C. J. Chang, P. J. Keller, and H. G. Floss. Biosynthesis of the ansamycin antibiotic assatrienin (mycotrienin) by Streptomyces collinus. J. Nat. Prod., 50, 108-118 (1987).

Yamazaki, M., Thorne, L., Mikolajczak, M., Armentrout, R. W. and T. J. Pollock. Linkage of genes essential for synthesis of a polysaccharide capsule in Sphingomonas strain S88, J. Bacteriol., 178, 2676-87 (1996).

Yang, K., Han, L. and L. C. Vining. Regulation of jadomycin B production in Streptomyces venezuelae ISP5230: involvement of a repressor gene, jadR2, Journal of Bacteriology, 177, 6111-7 (1995).

Yanisch-Perron, C., J. Vieira, and J. Messing. Improved M13 phage cloning vectors and host strains: nucleotide sequences of the M13mp18 and pUC19 vectors, Gene, 33:103-119 (1985).

Yoda, N., and N. Hirayama. Structure-activity relationships for mitomycins. Application of the distance and charge analysis method. J. Med. Chem., 36, 1461-1464 (1993).

Zhang, H. Z., H. Schmidt, and W. Piepersberg. Molecular cloning and characterization of two lincomycin-resistance genes, lmrA and lmrB, from Streptomyces lincolnensis 78-11. Mol. Microbiol., 6, 2147-2157 (1992).

While the present invention has been described in connection with the preferred embodiment thereof, it will be understood many modifications will be readily apparent to those skilled in the art, and this application is intended to cover any adaptations or variations thereof. It is manifestly intended this invention be limited only by the claims and equivalents thereof.

What is claimed is:

1. An isolated and purified nucleic acid molecule comprising a nucleic acid sequence comprising a mitomycin biosynthetic gene cluster, a variant or a fragment thereof.

2. The isolated and purified nucleic acid molecule of claim 1 which encodes MitT, MitS, MitR, MitQ, MitP, MitO, MitN, MitM, MitL, MitK, MitJ, MitI, MitH, MitG, MitF, MitE, MitD, MitC, MitB, MitA, or any combination thereof.

3. The isolated and purified nucleic acid molecule of claim 1 which encodes MmcA, MmcB, MmcC, MmcD, MmcE, MmcF, MmcG, MmcH, MmcI, MmcJ, MmcK, MmncL, MmcM, MmcN, MmcO, MmcP, MmcQ, MmcR, MmcS, MmcT, MmcU, MmcV, Mct, MmcW, MmcX, MmcY, or any combination thereof.

4. The isolated and purified nucleic acid molecule of claim 1 which is from Streptomyces spp.

5. An expression cassette comprising the nucleic acid molecule of claim 1, 2 or 3 operably linked to a promoter functional in a host cell.

6. A recombinant bacterial host cell in which at least a portion of a nucleic acid molecule comprising a mitomycin biosynthetic gene cluster is disrupted to as to result in a recombinant host cell that produces altered levels of mitomycin relative to a corresponding nonrecombinant bacterial host cell.

7. The recombinant host cell of claim 6 in which mitomycin levels are increased.

8. The recombinant host cell of claim 6 in which mitomycin levels are decreased.

9. The host cell of claim 6 wherein the nucleic acid molecule which is disrupted encodes MitT, MitS, MitR, MitQ, MitP, MitO, MitN, MitM, MitL, MitK, MitJ, MitI, MitH, MitG, MitF, MitE, MitD, MitC, MitB, MitA, or any combination thereof.

10. The host cell of claim 6 wherein the nucleic acid molecule which is disrupted encodes MmcA, MmcB, MmcC, MmcD, MmcE, MmcF, MmcG, MmcH, MmcI, MmcJ, MmcK, MmcL, MmcM, MmcN, MmcO, MmcP, MmcQ, MmcR, MmcS, MmcT, MmcU, MmcV, Mct, MmcW, MmcX, MmcY, or any combination thereof.

11. A recombinant host cell, the genome of which is augmented with at least a portion of a nucleic acid molecule comprising a mitomycin biosynthetic gene cluster operably linked to a promoter functional in the host cell.

12. The recombinant host cell of claim 11 in which mitomycin levels are increased.

13. The recombinant host cell of claim 11 in which mitomycin levels are decreased.

14. The host cell of claim 11 wherein the genome is augmented with a nucleic acid molecule that encodes MitT, MitS, MitR, MitQ, MitP, MitO, MitN, MitM, MitL, MitK, MitJ, MitI, MitH, MitG, MitF, MitE, MitD, MitC, MitB, MitA, or any combination thereof.

15. The host cell of claim 11 wherein the genome is augmented with a nucleic acid molecule that encodes MmcA, MmcB, MmcC, MmcD, MmcE, MmcF, MmcG, MmcH, MmcI, MmcJ, MmcK, MmcL, MmcM, MmcN, MmcO, MmcP, MmcQ, MmcR, MmcS, MmcT, MmcU, MmcV, Mct, MmcW, MmcX, MmcY, or any combination thereof.

16. A recombinant host cell comprising a mitomycin biosynthetic gene cluster, the genome of which is augmented by a recombinant nucleic acid molecule, wherein the recombinant nucleic acid does not comprise a mitomycin biosynthetic gene, and wherein the recombinant host cell produces a biologically active agent that is not produced by the corresponding non-recombinant host cell.

17. A product produced by the recombinant host cell of claim 6 or 11 which is not produced by the corresponding non-recombinant host cell.

18. The product of claim 17 which comprises a biologically active agent.

19. The product of claim 18 which is a mitomycin.

20. The product of claim 18 is not a mitomycin.

21. An isolated and purified nucleic acid molecule comprising a nucleic acid sequence which encodes polyketide biosynthetic enzymes or a fragment thereof, wherein the nucleic acid sequence hybridizes under hybridizing conditions to SEQ ID NO:74.

22. An isolated and purified polypeptide comprising MitT, MitS, MitR, MitQ, MitP, MitO, MitN, MitM, MitL, MitK, MitJ, MitI, MitH, MitG, MitF, MitE, MitD, MitC, MitB, MitA, MmcA, MmcB, MmcC, MmcD, MmcE, MmcF, MmcG, MmcH, MmcI, MmcJ, MmcK, MmcL, MmcM, MmcN, MmcO, MmcP, MmcQ, MmcR, MmcS, MmcT, MmcU, MmcV, Mct, MmcW, MmcX, MmcY, or any combination thereof.

23. An isolated and purified nucleic acid molecule comprising a nucleic acid sequence comprising sugar metabolism genes or a fragment thereof, wherein the nucleic acid sequence hybridizes under hybridizing conditions to a DNA comprising SEQ ID NO:75.

24. An isolated and purified nucleic acid molecule comprising a nucleic acid sequence which encodes an aminoDAHP synthase from Streptomyces strains that produce mitomycin.

25. A recombinant host cell in which at least a portion of a nucleic acid sequence which encodes polyketide biosynthetic enzymes is disrupted so as to result in a recombinant host cell that produces altered polyketide levels or polyketides of altered composition relative to a corresponding nonrecombinant cell, wherein the nucleic acid sequence hybridizes under hybridizing conditions to SEQ ID NO:74

26. A recombinant host cell in which at least a portion of a nucleic acid sequence which encodes sugar metabolism enzymes is disrupted so as to result in a recombinant host cell that produces altered sugar levels or molecules with altered sugar composition relative to a corresponding nonrecombinant cell, wherein the nucleic acid sequence hybridizes under hybridizing conditions to a DNA comprising SEQ ID NO:75.

27. A recombinant host cell, the genome of which is augmented with at least a portion of a nucleic acid sequence which encodes polyketide biosynthetic enzymes operably linked to a promoter functional in the host cell, wherein the nucleic acid sequence hybridizes under hybridizing conditions to SEQ ID NO:74.

28. A recombinant host cell, the genome of which is augmented with at least a portion of a nucleic acid sequence which encodes sugar metabolism enzymes operably linked to a promoter functional in the host cell, wherein the nucleic acid sequence hybridizes under hybridizing conditions to a DNA comprising SEQ ID NO:75.

29. An isolated and purified nucleic acid molecule comprising a nucleic acid sequence that hybridizes under hybridizing conditions to a nucleic acid segment comprising SEQ ID NO:96, or a fragment thereof.

30. The isolated and purified nucleic acid molecule of claim 29 which is plant nucleic acid.

31. The isolated and purified nucleic acid molecule of claim 29 which is prokaryotic nucleic acid.

32. A method to introduce exogenous DNA into a refractory Streptomyces strain, comprising:
a) contacting a bacterial donor cell comprising a conjugative plasmid with a Streptomyces cell so as to yield a transformed Streptomyces cell comprising at least a portion of the plasmid; and
b) identifying the transformed Streptomyces cell.

33. The method of claim 32 wherein the Streptomyces strain produces a mitomycin.

34. A method to identify a nucleic acid molecule that is related to at least a portion of a nucleic acid molecule comprising a mitomycin gene cluster, comprising:
a) contacting a sample comprising nucleic acid with an amount of a probe comprising at least a portion of a nucleic acid molecule comprising a mitomycin gene so as to form a complex;
b) detecting the presence or absence of the complex.

35. A method to identify a nucleic acid molecule that is related to at least a portion of a nucleic acid molecule comprising a mitomycin gene cluster comprising:
a) contacting a sample comprising nucleic acid with at-least one oligonucleotide under conditions effective to amplify the nucleic acid so as to yield an amplification product, wherein the oligonucleotide specifically hybridizes to nucleic acid comprising a mitomycin gene cluster, and
b) detecting or determining the presence or absence of the product.

36. The method of claim 34 or 35 wherein the sample is obtained from a plant.

37. The method of claim 34 or 35 wherein the sample is obtained from a microorganism.

38. An isolated and purified nucleic acid molecule comprising a nucleic acid sequence comprising a gene product selected from MitT, MitS, MitR, MitQ, MitP, MitO, MitN, MitM, MitL, MitK, MitJ, MitI, MitH, MitG, MitF, MitE, MitD, MitC, MitB, MitA, MmcA, MmcB, MmcC, MmcD, MmcE, MmcF, MmcG, MmcH, MmcI, MmcJ, MmcK, MmcL, MmcM, MmcN, MmcO, MmcP, MmcQ, MmcR, MmcS, MmcT, MmcU, MmcV, MmcW, MmcX, MmcY, or any combination thereof.

39. An isolated and purified nucleic acid molecule comprising a nucleic acid sequence encoding at least one gene necessary for mitomycin biosynthesis.

40. An isolated and purified nucleic acid molecule comprising a nucleic acid sequence encoding at least one gene for mitomycin transport.

41. An isolated and purified nucleic acid molecule comprising a nucleic acid sequence encoding a polypeptide that regulates mitomycin biosynthesis or resistance.

42. A method for preparing a compound or a pharmaceutically acceptable salt thereof from a recombinant host cell comprising culturing the host cell of claim 6, 11 or 16 in a culture medium containing assimilable sources of carbon, nitrogen and inorganic salts under aerobic fermentation conditions so as to yield an increase in the compound relative to the level of the compound produced by the corresponding non-recombinant host cell.

43. A method for preparing a mitomycin or a pharmaceutically acceptable salt thereof from a recombinant host cell comprising culturing the host cell of claim 6, 11 or 16 in a culture medium containing assimilable sources of carbon, nitrogen and inorganic salts under aerobic fermentation conditions so as to yield an increase in the mitomycin relative to the level of the mitomycin produced by the corresponding non-recombinant host cell.

44. A product produced by the recombinant host cell of claim 16 which is a mitomycin.

45. A product produced by the recombinant host cell of claim 16 which is not a mitomycin.

46. The product of claim 19 which is 9a-demethoxy mitomycin C.


SEQUENCE LISTING (153 sequences)

SEQ ID NO: 1 - protein, 115 aa, Streptomyces lavendulae
RIGAGSRVLDLGCGVGTPGVRIARLSGAHVTGISVSHEQVVRANALAEEAGLADRARFQRADAMDLPFEDESFDAVIALESIIHMPDRAQVLAQVGRVLRPGGRLVLTDFFERAP

SEQ ID NO: 2 - protein, 114 aa, Streptomyces lavendulae
RLAPGERVLDVGSGNGKATLRIAARHGVRATGVSINPYQVGLSRQLAEKEGDEATEFRIGDMLALPFPDGSFDACYAIESICHALERADVFTEIARVLRPGGRVTVTDFVLRRP

SEQ ID NO: 3 - protein, 115 aa, Streptomyces lavendulae
DFSGAATAVDIGGGRGSLMAAVLDAFPGLRGTLLERPPVAEEARELLTGRGLADRCEILPGDFFETIPDGADVYLIKHVLHDWDDDDVVRILRRIATAMKPDSRLLVIDNLIDER

SEQ ID NO: 4 - protein, 115 aa, Streptomyces anulatus
DFSSYGTVVDIGGADGSLLAAVLSAHPGVEGVVFDSPEGARDAAATLDAAGVGERGRVETGDFFTRVPGGGDLYVLKSILHDWSDARSADILRTVRAAMPAHARLLVVEVLLPDT

SEQ ID NO: 5 - protein, 117 aa, Streptomyces glaucescens
GMERFSRIADLGGGDGWFLAQILRRHPHATGLLMDLPRVAASAGPVLEEAKVADRVTVLPGDFFTDPVPTGYDAYLFKGVLHNWSDERAVTVLRRVREAIGDDDARLLIFDQVMAPE

SEQ ID NO: 6 - protein, 115 aa, Amycolatopsis mediterranei
PLRAGDRLLDIGCGNGEPAIRMATANDVMVTGISISEKQVERANDRAYKADVDDRVVFEYADAMELPYPDASFDVVWALESLHHMPDRWHVIRQAARVLRPGGRLALGDFLLVPS

SEQ ID NO: 7 - protein, 116 aa, Saccharopolyspora erythraea
GISEGDEVLDVGFGLGAQDFFWLETRKPARIVGVDLTPSHVRIASERAERENVQDRLQFKEGSATDLPFGAETFDRVTSLESALHYEPRTDFFKGAFEVLKPGGVLAIGDIIPLDL

SEQ ID NO: 8 - protein, 120 aa, Artificial Sequence
  A consensus sequence
XXXXGXRXLDXGXGXGXXXAXXXXXXXGXXXTGXXXXPXXVXXAXXXAEXAGVXDRXXFXXGDXXXLPXPDGXFDXVYXLESXLHXXXRXXXXXVXRXXXXVLXPGXGRLXXXDXXXXXX

SEQ ID NO: 9 - DNA, 3765 bp, Streptomyces lavendulae
ggatccgagggccggagtgggattcggctcaatgaaccatgcacacagcacataccaggacggtgtcgcgcccaccatacgcgacgcttcccgctccctccagccgtgcggtttgagccacttcgacgccggataacgttgccgacaggcccgccgagcagcccctgaactggatcaattcccttgggaataaggcagtttcactgctcaaccaccctgctgacgagaatccaccgccgaccggcggtcggggcagaccttcccggcaagggtgttgactccggcaactgccctatggaggctcgtgtctggcatccgatcccggcctatgaccgggggccggatcacatgcccgctccggccacccctcacaccgcgggccggatttcccgccgcccccgaggaacggcgtttcccgtcgggtcacgcaccacccttcccgacgcggggcgaacacaacggaaccgggccgtgaagccacggccaccgaaggcaaaggcctcgacacccgccctcccgccgtacagcgccccgaagtcgaccgtgccgccgcacccgcaggaccgaaaggctgctcaatgacacctacgtccggtgatgacgtcctgtcctttccctcatggccgcaacacggcgcggaggagcgcgccggactcctgcgggccctggaccagaaggggtggtggcgcgacgcggggcaggaggtcgatctcttcgagcgggagttcgccgaccaccacggcgccccgcacgcgatcgccacgacgaacggcacccacgccctggaactcgccctgggggtcatggggatcggccccggtgacgaggtcatcgtccccgcgttcaccttcatctcgtcgtcgctggccgtgcagcgcatgggcgcggtgccggtgccggcggacgtacggcccgacacctactgcctcgatgccgacgcggcggcggcgctggtgacgccacgcaccaaagcgatcatgccggtccacatggcgggccagttcgccgacatggacgccctggagaagctctccgtcgcgacgggcgtgccggtcctccaggacgccgcgcacgcccacggcgcgcagtggcagggccgccgggtcggggagctcggctcgatcgccgccttcagcttccagaacggcaagctgatgaccgccggcgagggcggcgccctgctcctgccggacgacgagtccttccacgaggcgttcctccagcactgctgcggccgcccgcccggggaccgcgtctaccgccatctgacgcagggctccaactaccgcatgaacgagttctccgcgagcgtcctgcgtgctcaactgaagcgcttgaaggatcagttgcgcatcagggaggagcgctgggcccagctgcgtacggcactggccgccatcgacggcgtggtgccgcaggggcgcgacgagcgcggcgacctccactcccactacatggccatggtccggctgcccggcatctcggcccggcgccgcctcgcgctggtggacgcgctggtcgagcggggagtgcccgcgttcgtcggcttcccgccggtctaccgcaccgagggtttcgcgcgcggcccggcgccggcggacgccgaggagctggccaagagctgtcccgtggcggaggagatcggcagcgactgcctctggctgcaccatcgcgtcctgctcgccgacgtgaccacgctggaccggctggcggaggtcttctccggcctcgtcggcgcgctctgacccgatgcgggcccccaacggcaccaccgccccccggctgagcgtcgtcgtccccagccgggggcccgcggcacgcctgcgcgcgaccctcgcatgccttgccggcccctccccgggaacgccgcccttcgaagtggtcgtcgtcgacgacaacgacgggggcgacgccggtgatcaactgatcgccgtgacaggcgagatgagcggccttctcccgctgcgcgtggtgcggggaccgctgcgggggcgggccgccgcccggaacgccggggcggccgcggccctcgcgccccggctggtcttcctcgacgacgacgtcctggtggggcccggcttcctcgccgcacacgccgcggccgcggaaccggacgccttcacccacggccggctgcgcgaactccccaccgcggcgcggttcctcgccgctgtcgagaaggccgccccgaccgaggtccgccgcgcccgcgccggactcgaacccgctgccccggccgcctccgagcggcgccaaccgcaccggcggctcgtcgccaacgccctggagcgggccgtggaggccatggccggcggctccctgccggacgtcgccccctggctcggcttcatcggcgcgaacaccgccctcgacaaggccgcatgggagcataccggcggattcgacgaggagttcgggctcacctgggggtgcgaggacctggagttcggcttccgcctgcacgccgccgggctgcgcaggaccctcgcccccgacgccctcggtgtgcacctcagccacgcccgccccggccgctgggagcagcaccaccgcaacctcacgcacttctccgccggccacccgcacccgtcggtacgcgccttggaggccctgctcgggcccgacggcacgccggaggcgtatgtgcgcgccgtcctggccgaagaggccgcaccggcacgggacgcggcgcgatgagcggcacaccggccaccgcgccgtacggtcccgtggtgctctccccgcacgcggacgacgccgtgtggtccctgggcgggcggctggcgcgctgggccgccgagggcccgcggccgaccgtcgtcacggtcttcgccgggcccgcggccgggaagcccgagtcgtggcggagcgccgccgatcccgcggtgcgccgggccgaggaccgggcggcatgtgccgaactgggcgtgcgccacgtgccgctgggcttcaccgacgcggcactgcgtacggcctcgggcgcctatctctacgcttccccgcgccggctcttcggcccctggcacccggccgacctcccgctgctggaggaggtgcgggcggctctgctgccgctgtgcgcgggggcgtcgagcgtccacgttcccctggcggcgggccggcacgtcgaccaccgcctggtccgcggcgcggtggagcccctgtcccccgcccgtaccgtcttctacgaggacttcccctaccggctgcgcgaacgtgaccacacgaacctgcggccgcgcacggaacggctgccgtccgaggcggtggaccgctggctgaccgccgccggtcactactccagccaggcgagcgcccacttcggcggtgcggccgccctgcgcgaggccctgttcgcccgcgcccgcgcacacggcgggcccggccggcccggccacgccgaccgccactgggtgcccgtcggccaggacgaccggggcgaggcccggccggcacccgtggaaagggggccgtgacccacgccgtgcgcagccccaccacgagagaggccactcatgtcccgtagcacccacccgccgacagccacccccgacgcgggcaccaggcgacgcctgccgctgatcggcaacgacctggtcatcaacgaggactcctgcaacctcagctgcacctactgcctcaccggacagagcaacctcaaggagggccactcccttcaactgatcttcgagcccccgcggcgcgacagctacgccaaggacagcgggctggggcagcgcatggacaaggtcgccgaccggatcc

SEQ ID NO: 10 - protein, 388 aa, Streptomyces lavendulae
MTPTSGDDVLSFPSWPQHGAEERAGLLRALDQKGWWRDAGQEVDLFEREFADHHGAPHAIATTNGTHALELALGVMGIGPGDEVIVPAFTFISSSLAVQRMGAVPVPADVRPDTYCLDADAAAALVTPRTKAIMPVHMAGQFADMDALEKLSVATGVPVLQDAAHAHGAQWQGRRVGELGSIAAFSFQNGKLMTAGEGGALLLPDDESFHEAFLQHCCGRPPGDRVYRHLTQGSNYRMNEFSASVLRAQLKRLKDQLRIREERWAQLRTALAAIDGVVPQGRDERGDLHSHYMAMVRLPGISARRRLALVDALVERGVPAFVGFPPVYRTEGFARGPAPADAEELAKSCPVAEEIGSDCLWLHHRVLLADVTTLDRLAEVFSGLVGAL

SEQ ID NO: 11 - protein, 272 aa, Streptomyces lavendulae
MVVVDDNDGGDAGDQLIAVTGEMSGLLPLRVVRGPLRGRAAARNAGAAAALAPRLVFLDDDVLVGPGFLAAHAAAAEPDAFTHGRLRELPTAARFLAAVEKAAPTEVRRARAGLEPAAPAASERRQPHRRLVANALERAVEAMAGGSLPDVAPWLGFIGANTALDKAAWEHTGGFDEEFGLTWGCEDLEFGFRLHAAGLRRTLAPDALGVHLSHARPGRWEQHHRNLTHFSAGHPHPSVRALEALLGPDGTPEAYVRAVLAEEAAPARDAAR

SEQ ID NO: 12 - protein, 260 aa, Streptomyces lavendulae
MSGTPATAPYGPVVLSPHADDAVWSLGGRLARWAAEGPRPTVVTVFAGPAAGKPESWRSAADPAVRRAEDRAACAELGVRHVPLGFTDAALRTASGAYLYASPRRLFGPWHPADLPLLEEVRAALLPLCAGASSVHVPLAAGRHVDHRLVRGAVEPLSPARTVFYEDFPYRLRERDHTNLRPRTERLPSEAVDRWLTAAGHYSSQASAHFGGAAALREALFARARAHGGPGRPGHADRHWVPVGQDDRGEARPAPVERGP

SEQ ID NO: 13 - protein, 386 aa, Streptomyces collinus
MSSGVQLGSAFRVWPQYDDAERTGLIRALEQGQWWRMGGGEVERFEREFAEYHGGEHALAVTNGTHALELALEVMGVGPGTEVIVPAFTFISSSQAAQRLGAVVVPVDVDPETYCIDPAEAAKAITPRTRAIMPVHMAGQLADMDALEKVAADSGVPLIQDAAHAQGATWNGRRLGELGSVAAFSFQNGKLMTAGEGGAVLFPTAEMAEHAFLRHSCGRPRNDRGYFHRTSGSNFRLNEFSASVLRAQLARLDGQIRTREERWPLLSSLLAEIPGVVPQRLDRRPDRNPHYMAMFRVPRITEERRARVVDTLVERGVPAFVAFRSVYRTDAFWEMGAPDLSVDELARLPPLRGLTTDCLWLHHRTLLGTEEQMHEVAAVIADVLGS

SEQ ID NO: 14 - protein, 388 aa, Actinosynnema pretiosum
MGSSPDAGIDFPAWPQHDDAERAALLRALDQGQWWRVGGSEVDEFEREFAEYHGAGHALAVTNGTHALELALQVLDVGPGTEVIVPAFTFISSSQAVQRLGAVAVPVDVDPDTYCLDVAAAEDAVTSRTSAIMPVHMAGQFADMDRLDKLSASTGVPVVQDAAHAHGAHWRGKRVGELGSIATFSFQNGKLMTAGEGGAVLFADQAQWEKAFVLHSCGRPKGDRGYFHLTSGSNFRMNEFSAAVLRAQLGRLDSQIATRQARWPVLSALLAGIDGVVPQTVDPRSDRNPSYMAMFRMPGVTEERRNAVVDELVRRGIPAFMAFRAVYRTQAFWETGAPDLTPEELAARCPVSEEITRDCVWLHHRVLLGAEEQVRRLAAVVADVVAGA

SEQ ID NO: 15 - protein, 388 aa, Amycolatopsis mediterranei
MNARKAPEFPAWPQYDDAERNGLVRALEQGQWWRMGGDEVNSFEREFAAHHGAAHALAVTNGTHALELALQVMGVGPGTEVIVPAFTFISSSQAAQRLGAVTVPVDVDAATYNLDPEAVAAAVTPRTKVIMPVHMAGLMADMDALAKISADTGVPLLQDAAHAHGARWQGKRVGELDSIATFSFQNGKLMTAGEGGAVVFPDGETEKYETAFLRHSCGRPRDDRRYFHKIAGSNMRLNEFSASVLRAQLARLDEQIAVRDEPWTLLSRLLGAIDGVVPQGGDVRADRNSHYMAMFRIPGLTEERRNALVDRLVEAGLPAFAAFRAIYRTDAFWELGAPDESVDAIARRCPNTDAISSDCVWLHHRVLLAGEPELHATAEIIADAVGRA

SEQ ID NO: 16 - DNA, 1800 bp, Streptomyces lavendulae
atggaggaccgcaagcgcgaggggtatttctagcgcggcggggccggtgcggcccacaagcggaggactagtccctaagtatgaagtcccctactccgtttgtctgttgagggcaggggcgccgtctgaggatgatgcagtccatgtcacagttactttccgggaaggacggcgcccaggaggcgccaagtcgcggcgggtccacgtgggtggcggtcctcgccgcgtgcgtggggcagttcgtggtggtcctcgacgtgtccgtcatcaatgtcgcgctgccgtcgatccgttccggcctcgacatcggcgagacgggcctgcagtgggtggtcaacgcctacgtcatcgccttcgcgggcttcctgctgctcggcggccgggcctccgacctcttcggccgcaaggccgtgttcgtcttcggcctcggggtgttcaccgccgcgagcctgctcggcggcctcgcgcaggcgccgtggatgctcatcgtcgcccgcgccctgcaaggcatcggggcggccgtgctctcacccgccaccctcgcgatcctcaccaccacgttccccgagggtccggcgcgcatcaaagccgtcgcgatctggacggccgtgggcacgggcggcggcgcggccggcggcctcatcggcggcctgctcaccgactacctctcgtggcgctgggtgttgctgatcaacgtgccgctgggccttgtcgtgatcgtcgcgaccgtcgcctggctggccgagagccgcagcgaccaggcacaccgacgccggctggacctcccgggagcggtgctggtgaccctgggcgtcggcagcctggcctacggcatctcgcagagcgagggccacggctggggctcgccgcggacgctcaccttcctgatcgtcggtgtcgtggcgctcctcgccttcgtcgccgtggagcagcgcacgcgcgagccgttgatgccgctcggtgtcttccgggtgcgctcggtgtcggcggccaacgccatcaccatcgtcagtggcatgggcttctacgcgatgtggtacttcctctcgctctacatgcagaacgtgctgaaatactccgccgtacagaccggcctggccctgcttccccacaccgccaccatcatcctctccgcgcagttcgcaccccgcctgatgcggtggatcaaggggcgcaccctcctcgtgatcgcgggactgctgaccgccgcgggcttcatctggcaggggaacatggacgccgacggctccttcctggcgaccctgctcggcccgggaatcgtcttctccttcggcgcgggcctgatgatgacgctcctcgcggtctccgccacgacgggcgtggagctctccgaatcgggcctggtggccggcctcgccaacacctcgcgcaccatgggcggcgcgctcggcctgtcggtcctcgcgtccgtcgccgcccgccgcacggccgacgtggggcccggcgcggagggcctggcctccggctacggtcgggcgttcgtcgtgtccggggccatcatcctcgtgagcatgctgatgatccccttcctgcccaagccccagccccagaccccggcggaatgacctgtgagcacggacatacgaggaggcttcgtggggcaggacagccggccgcggtggctcaccgacgaggaacaacgcgtgtggcgcggctatctgcgggccaccaggctggtggaggaccacctggaccgccgcctccagcgggaagcggacatgccgcacctctattacggtcttctcgtccagctctccgaggccccgcgccgggggatcc

SEQ ID NO: 17 - protein, 484 aa, Streptomyces lavendulae
MMQSMSQLLSGKDGAQEAPSRGGSTWVAVLAACVGQFVVVLDVSVINVALPSIRSGLDIGETGLQWVVNAYVIAFAGFLLLGGRASDLFGRKAVFVFGLGVFTAASLLGGLAQAPWMLIVARALQGIGAAVLSPATLAILTTTFPEGPARIKAVAIWTAVGTGGGAAGGLIGGLLTDYLSWRWVLLINVPLGLVVIVATVAWLAESRSDQAHRRRLDLPGAVLVTLGVGSLAYGISQSEGHGWGSPRTLTFLIVGVVALLAFVAVEQRTREPLMPLGVFRVRSVSAANAITIVSGMGFYAMWYFLSLYMQNVLKYSAVQTGLALLPHTATIILSAQFAPRLMRWIKGRTLLVIAGLLTAAGFIWQGNMDADGSFLATLLGPGIVFSFGAGLMMTLLAVSATTGVELSESGLVAGLANTSRTMGGALGLSVLASVAARRTADVGPGAEGLASGYGRAFVVSGAIILVSMLMIPFLPKPQPQTPAE

SEQ ID NO: 18 - DNA, 990 bp, Streptomyces lavendulae
atcccgatcgtctcggacatgaccggcgaccttctcggcgcgcgggaggcccaggaccccgcctactgggtgtcccacatccgccgcgcggtgcgcttccacgaccagatccgccgtctgcagcgctacggggccggggccttcgtcgaggtcggcccggacacggtgctcagctcggccggccaggcgtgcctgacggaccaggcgggcaggagcgcgcccgtcctggtgtccctcgcgcacgccgagcgcgcggaggtgcccgcgctcctgaccgctctggccaccctgcacacccgtggcgtggccgtggactggcgggcgtggttcggcgacgggccgcgcgcggccggcctgcccacatacgcgttccagaagcagcactactggccgtcgggccccaccggttggcggtccgggcccgcccccgtacccctgccccaggccggaacggaggacgccgaaaggcccggtcgcgccgcggagtggcgggcgctgccgcccggtgagcggtacgacgcgctgctgcggatggtgcgcggcgaagccgccgccgtgatggggcacgccgggccggaggcggtggagccggagcgcggcttcctcgaccacggcttcgactcggtgatggccgtgaagctgcgcgaccgtctcgtggccgggacggggcgggagctgccgacgaccctgctgttcgaccaccccacgcccgcggccgtcgccgactacctgctggcggggacgggcgaggccgagacggcgccgtccgtgtccctgtcggaccagctcgaccgcctggaggccgacctcgcgcggctgccggccgacgaccggcagcgcgcccgcgtcgccgagcggctcaagggcctgctcgcggtccacgcgccggaccggggcgccgggagcgaggacgcgccggaccaggacgcgctggacacggcgaccgacgacgagatgttcgagctgatcgagaaggaactccgccgtgga

SEQ ID NO: 19 - DNA, 3978 bp, Streptomyces lavendulae
gtggatgagaccaacgagaccaaactccgcgagtacctgcggctggtcacggccgatctgcggcgaacccgcaggcagttggaggaggccgaggacgcggcccgcgagcccgtcgcgatcgtgggcatggcgtgccgcttccccggggacgtggcatcgccggacgacctgtggcagctggtcgccgagggccgggacgccgtcaccgagttccccgccgaccggggctgggacgtcgacgccgtctacgaccccgagccgggcaccccgggcaggacgtacgcgcgccacggcggcttcctcaaggacgccgccggattcgacgccgccttcttcggcatcacgccgcgcgaggcgctcgccatggacccgcagcagcgcatgatcatggaggtctcctgggaggcgttcgagcaggcgggcctcgacgcgaccaccctgcggggcgaggacgtcggcgtcttcgtcggctccaacagcaacgactacctgatcaacgtgctcgacgcgcgggacgtcgccgagggcttcatcgggaccggcaactccgccagcatcctctccggccgcgtcgcctacaccttcggcttcgagggcccggccgtgtccgtcgacaccgcctgctcctcctcgctggtcgcgctgcacctggccgcgcagtccctgcggcagggggagtgctccctggcgctggcgggcggcgcgacggtgatggccacgccgaccgccttcatcgagttcagccgccagcggggcctggcccccgacggccgctgcaagtccttctcggcgaccgccgacggcaccacctggtccgagggcgcggccgtgctgctgctggcccggctctcggacgcccgccgcctgggctaccccgtgcacgcggtcatccggggcagcgccgtcaaccaggacggcgcgagcgcgggcctgaccgcgcccaacggaccggcgcaacagcgggtgatccggcaggcactggccaacgcacggctgacggccgacagcgtcgacgcggtcgaggcacacggcaccggcaccccgctgggcgacccgatcgaggcccaggccctcctcgccacctacgggcgggcccgcggcgagggcaggccgctgtggctgggctcgctgaagtcgaacctgggccacacccagtccgcggccggcgcgggcggcgtcatcaagatggtgatggccatgcggcacgggacgctgccccgcacgctgcacctcacggagcccaccccgcgcgtcgactggtccgccggtgacgtacggctgctgaccgaggcccaggactggccggacaccggacagccgcgccgtgcggccgtctcgtccttcggcgtcagcggcaccaacgcccatgtgatcctggagggcccgcccgccgaggaggcaccggacgcgccgctgccggacgtctcctcgcagccgcggggcccgctgccgtgggtcgtctccggccgcagcgaggcggccgtccgagcgcaggccgagcgcctggcggcccacctgaccgcgcgcccgcacctggcaccggccgacgtggccaccgcgctggccaccacgcgggcggccttcgaccaccgggccgccgtcgtcggccgggaccgtgaggaactgctcgccggcctcgcggccctggccaccggaacccgcgcgcccggcctggtcaccggccggaccccgccgtccggcggcaaggccgccttcctcttcaccggacagggcagccagcagcccggcatgggccgcgaactggcggctcacagcaccgtgttcgccgacgccctggacgaggtctgcgcccagctcgaccggcacctcgaccggccgctgcgcgaggtgctgttcgccgcggacggcacgcccgaggccgccctgctcgacacgacggcctacacccagcccgcgctgttcgccgtcgaggtcgcgctgctgcggctgctggaggactggggcttgcggcccggcatggtcgcgggccactcggtcggcgaactgaccgccgcctacgccgccggggtctggtcgctcgccgacgcctgcgccctggtcgccgcccgcggccggctgacccaggcactgcccgcgggcggcgccatggtcgccgtgcaggcgaccgaggacgaggtgcgcgcccaactcgccgacggccgccccggcgtggacatcgccgccgtcaacggaccggaagcggtggtgctgtccggcgacgaggccgccgtcacggacctggcgcgcgagtgggccgcccgcggccgggagaccaggaggctgcgggtcagccacgccttccactccgcccacctggacgccatgaccgaggcgttcgccgaggtcgcacgaggggtgtcctacagcgcgccgtccctcccggtggtctccacgctcaccggggcccccgtcaccgacgagctccgcaggccggaacactgggtgcggcacgtccgggagacggtgcgcttccacgacgcggtccgcgccctgcgcgaccgcggggccaccgcgttcctggaggtcgggcccggcggcgtgctgacggccgcggcacgccgatgcctgcccgacgccgcccccgagacgttcgtccccgtgctgcggcgccgcaggcccgaacccgagtccgtgctgacggccgtcgcgcaggcccacacgatcggcctctcgccggcgtgggaccgcctgctgcccaaggcccggacgcgcgtggacctgcccacgtacgccttccagcgcggccactactggctggcgggcatggccggagcgggcaccgcgcggccggtgcggccggaagtgcaggagcccaccgccccctccggtacgccgccgctgtcgcgacggctggccgacgcgtcggaggaggagcgcggccacctgctgctgacgctggtacgcgagcagtcggccaccgtgatgggcggcgtcgaccccgcgcaggtcgaacccgaccgccccttcctggagctcggcttcgactccctgatgggcgtcgagctgcgcaccgcgctcgccgccgactgcgcactgcccctgccgcccggcctgatcttcgaccaccccacgcccgccgccctggccgccttcctcggcgagcagctcgcggcggcggcctccggcacccccacggcggcggcaccctcgccgtactccctggaggcgctgtaccgcaacgccaacaccctcgaccggcccgaggacgcgctcgccctcaccaaggccgcctcccggctgcgcccggtcttcgccagcgtggccgaggcggggcaggacccggtcacggtggagctggcacaggccaccggccttccgggcctgatctgctgcccggcacccgtgccgctgtacggggcacagcagtacagccggctcgcagccgccttccgcggcacgcgcggagtctcggccctgctcgcccccggcttctccccgggcgaactgctgcccgccgacttcgaggtgatgcaggacttcctcgccgagggggtccggcggcagaccgacggcgcgcccttcgtcctcctgggccactcctccgggggctggttcgcctacagcctggcggcccacctggcgcgcaccgggccgcgcccggaggccgtcgtgctgctggacacctatcagctgcacgacccggcgctgcaccgcatgcagcgcgaactcgcccagggcgtcctggaccgcgaggaggacttcggggcgatgacggacgtacggctgagtgccatgggcaaatacttcgacttcttcaccgactgggtggccgaggacgccggtgtcccgacgctgctgctgcgggcctccgagcctctgggcgaggtcgtcgagggccaggagtggcgctccacctggccgttcgacagcacggtcctcgacacggaaggcgaccacttcgccatggtcaacgaccacgcgccgcggacggcccaggccgtgaacggctggctgtcgggcctcaccggcggaaggggc

SEQ ID NO: 20 - DNA, 570 bp, Streptomyces lavendulae
gtggagacacgcaacgccgaacggccgtggatacgcagcttccaccccgctccccaggcccctgtgcggctgctgtgcctgccgcacgccgggggctccgcgagcgcctacttcgcgctgtcgagggaactggcgccccgggtggaggtgctcgccgtgcagtaccccgggcggcaggaccggcgcgacgagccgctgctggactcgatcgaggccctgcgcgacggggtcgccgaggccctgacgccctggctggaccggccggtcgccctcttcggccacagcatgggcgccgtggtggcctacgagctggcgcggctgctgtgccaggacgcgggcgtgccgctcacccacctcttcgtctccggacgccggggatccgaccgaagtctccgtccttgccgccgtgttccggaattcaccgtgacaccgccgcgcggctcttcttccgaagtcctccagatccggcacgagtttgtatccgaacggggttctgcgtgcgaaatactctcttcgaattgggtgacatacccccgatcggcaccgtacccgagcagatgtacgcctcgg

SEQ ID NO: 21 - DNA, 1245 bp, Streptomyces lavendulae
gtgcgaaatactctcttcgaattgggtgacatacccccgatcggcaccgtacccgagcagatgtacgcctcggtgatccgacgggagcgctacggacagccccaccaggcgttccgcagcgaggtcgtggacgtgccgaaggtggggcccggtcaggcgctggtcctcgtgatggccgcgggcatcaactacaacaacgtctgggcctccctggggcagccggtcgacgtgatctccgcgcggcagaagcagggccacagcgaggacttccacatcggcgggtccgagggctccggcgtggtgtgggcggtgggggagggcgtcacccaggtcgcggtgggcgacgaagtgatcctctccggctgccagtggacggagacggccgccgacatccggctcggcgccgaccccatgacctccggctcgcagtcggtgtggggatacgagggcaactacggctccttcgcccagttcgccctcgtcgacgactatcagtgccaccccaagccgcccggcctgacctgggaggaagccgcctgcttcctgctcaccggggccaccgcctaccgccagctgtgcggctggcagccgcacgacgtgcggccgggcgacccggtcctcatctggggcggggccggcgggctcggctccatggccatccagatcacccgggcgcggggcggcatccccgtcgccgtggtctccgacgaggagcgggcccgctactgccgggagctcggcgcccagggcaccatcaaccgcctggacttcgaccactggggacggctgcccgacatcggcgaccacgaggcgatgggccgctggaccgagggtgtacgggccttcggccggcgcttctgggaggtgctgggcgagcgcaggtccccgcgcatcgtcctggagcacagcggccaggccaccatccccacctcgatgtacctgtgcgacaacgcgggcatggtcgtcatctgcggcggcaccaccggctacaacgccgacatcgacctgcgcttcctgtggatgcgtcagaagcgcttgcagggctcgcacttcgccaacctgcggcagtgccgcgacgtcatccacatggtcgcgaacggccagctcgacccgtgcctgtcgtggaccggcggcttcgacgacatcggcaaggcacaccagctgatgcacgacaaccagcacccccagggcaaccaggccgtcctggtcaacgcgccgcggaccggcctgaccaccttcgcctga

SEQ ID NO: 22 - DNA, 1224 bp, Streptomyces lavendulae
gtgtccgacaccgagcagcacgcgcccacgctgccgcggcagcgcacctgccccttctcgccgccgcccgagctcgaggagctgcggcgcaccgatcccatcagcaggatgcggttcgccgacgactccccgggatggctgctgacccgccacgccgacgtccgcgccgcgctggccgaccccggcgtcagctcgcaccccggcaaggcaccccagccctggcgcaacctcgcccccgagatgcgcgccgagcactacctgccgggcttcctgatcttcatggacccgccggaccacacccgctaccgccgcctgctcaccaagtggttcaccatgcgggccatccgcaagctcgaacccaggatcgagcagatcgtcaccgagaccctcgacgccatggaggcccagggcggcaccgtcgacctggtgcagtccttcgcgctgccgatcccgctgctggtcatctgcgagctgatgggcatccgctacgaggagcgcgaggagttcatggacatggtcctgcgactccaggccctggacgccacgcccgaggaactcggggccctcggcgccaggatgaacgagttcatgatgaagctcgccgccgccaagcgcgcgaaccccggcgacgacctgctcagccacctcgcccacgaccccgacgccgacccggcgctcacggatctggagatcgccggcatcggcgtgctgatgctcatcgcggggcacgagacctcggccaacatgctgggcgtcggcacctacaccctgctggagaacgccgaccagtgggccctgctccgtgacgacatcagcctgatcgaccgggccgtcgaggagctgctgcgccaccagaccatcgtccagcagggcctgccgcgcggcgtcacccgggacatggagatcgccgggcaccaggtgaagaccggggagtccctgctggcctcgctgcccgccgccaaccgcgaccccgccgtcttccccgaccccgaccgcctcgacatcacgcgcgagcacaacccgcacctcgccttcggccacggcatccacctctgcctgggcatggagctcgcccgggtggagatgcgccaggcgtggcgcggcctcgtcacgcgcttccccggcctgcgcatggccgccgcgcccgaggacatccgctggcgcgacgaccagatcgtctacggcgtgtacaacctcccggtgacctgggacgaggccaagtga

SEQ ID NO: 23 - DNA, 531 bp, Streptomyces lavendulae
atggacaagctcgacatcctctggagcgagcgcgagatccgtgccgtgctgcagcgctactgccgcgggctcgaccgcctcgacgaggaactggtcaagtccgcctaccacgaggacgcgcacgacgaccgcggcgtcatccgcggcaacgcacacgacttcgtcaagcagatcgtcccgctcctgcgcgacgcctacaccggcaccctgcacaccctgcacggcagcacgatcgagatcgacggggatgccgcgggcgtggagtcctactgcaccgcctaccactaccgcgagagcgacggcatcaagcgggtggagcagttcgccgggcgctacgtcgaccgcttcgagcggcgcgacggcgtctggaagatcgcccgccggctcgtgctgaacgacttcagcctcgcccaggaggtgccgctcgaccccgccgaggcccaggccggcttcaacccctcccaccgcgacctcaccgacgccagctaccaggtgctgccgctgcgcggcccggacgcccccaccctctga

SEQ ID NO: 24 - DNA, 1233 bp, Streptomyces lavendulae
gtgaccggccccgaggccgcggtgcgcgggtgccccttcggcgccggcgaggcgcccgcgtaccccttccacgcccccgaccggctggagcccgacccgtactgggagccgctgcgccgcgagcggccgctgcaacgcgtcacgctgccgtacggcggcgaggcgtggctcgccacccgctatcaggacgtgcgcgcggtcttcgccgaccgcaggttctcccggcagctcgccgtcgcgcccggcgctccgcgcttcctcccgcaccagccgccgccggacgccgtcctgagcgtcgagggccccgaccacgcgcggctgcgccggctggtcgggaaggtcttcacgccgcgccgcgtggaggacatgcgtccgctcatccagcgcaccgccgacggactcctcgacgcgatggaggagatggggccgcccgcggacctggtcgaggacttctccctgcccttcgccgtgtccatgatctgcgagctgctcggcgtgccgcccgaggaccgcaagcggttctgcgtctggtcggacgcgctgctgacgaccaccgcgcacacccccgcccaggtgcgcgactacatgatgcagatgcacgactacctcggcgggctcgtcgcgcagcgccgggtgcggcccaccgcggacctgatcggctccctcgtgaccgcgcgcgacgaggaggacaagctcaccgagggcgagctggtgcggctggccgaggccatcctcatcgccggctacgagacctcggcgagccagatccccaacttcctctacgtcctcttccgccacccgcagctgctggagcggatcaggaacgaccacgacctcatccccgacgccgtcgaggaactgctgcgcttcgtgcccatcggcaccgtggacggctttccccgtacggccaccgaggacgtcgagctcgggggagtcctggtcagggccggggagacggtcgtgccgtcgatgggcgccgccaaccgcgaccccgagctgttcacggaccccgacgagctggacctcgcgcggcggccgaatccgcacctgggcttcggcgcgggaccgcaccactgcctgggcgcccaactggcccgggtggagctccagatcacgctcacgacgctgttccgcagatacccccgcctgcggctggccgtgccggaggagagcctctcgtggaaggaggggctgatggtccgcggcatgcacaccatgccggtcacctggtga

SEQ ID NO: 25 - DNA, 1107 bp, Streptomyces lavendulae
atgagcaccatcgacgaatgggaacacagcacgaaggaggcgggcatggaccccgcggccctcagacgcctgaccgatgtggtgcgggcgaggggcggcgcggcgcagctgtgcgtcatgcggcggggcaccgtggtcctggaccgctcgttcggctgctcctccgactccctcttcctcgtctacgcggccaccaagcccgtcgccgccctcgccgtgcacgcgctcgccgagcggggcctgatcgggctggaccggccggtggccgaatactggccgcagttcgcccggcacggcaagggtgacgtgaccgtccgtcatgtcctccagcaccgggccggggtgccggtcggccggggcatcgtgcgcacgatgcgcaccgccggcgactgggagcgctccgtgcgcgaccttgagcagtcccggcccaagtggcccggcggcgaggtcgccgcctaccacttcatgagtttcggattcattctcggcgaactggtgcagcgcgtcaccgggcggtcgttccgagatttcgtgacttccgagctcttcgccccacttgggctgaatgatttgcacatgggattgcccggcagtgcctggccccggcatgtgcccgcgcgggccgcccacccctccgaatggcccaatcagtggatgagcaaccgccgcggctaccgccaggccgtcattccgtccgccggtctttccggaaccgccgcacaaatggcccgcttttaccagatgcttatggagggcggctcgctcgacggcatccgcgtgctgcggcccgaaactgtggaggaagccagaaaaccgtccaatgacggcggaatcgacgcttccctcaagcgtccggtccgctggtcccacggattcatgctcggtggtccgggcccggacccgcgggggctgtccaatgtgctgggccgcacgagcgacccgagcgccttcgggcacgcgggcaccacgtccagcgtcgtgtgggccgaccccacgcgcgagctggtcctcgcctacctctccaacatccagcccgagttcggagcgggtatcgagcggctccgcgaggtcagtgacctcgcgctcggtgcctgcgaggcaggctga

SEQ ID NO: 26 - DNA, 858 bp, Streptomyces lavendulae
gtgctgaatctgcccaaaggaatggagcgcgcgcatccgcattctccgccacaggtgggaatactcggacccttggaagtccgctcggccggaggtgccggaacgggagccgcggtaagcggtattcgcgtacgcacattgcttgccgcgttgactgcccgcctggggcaggcgatgtcgaccgagcgcatcctcaaagaggtctgggccgacaacccgcccgcgaccgatcgcaaggcggtggccgtcgccgtcctgcggctgcggcgggtcctcggcgacaacgaaggacggtggctgctcacccgcccctccggttacgtcctggacatccccccggaccacctcgacgccgtacgcgcggagaccctggtgcgggaaggccgggccgccctggccgccggcgacccacgcgtcgcggcccgccacctcacgcgcgccctcgaccagtggcggggcgagccctacgcggacgccaacgccatctcgaccgtgtcccagcgcatcacggagctggagaacctcaggtccgaggccgtccaggcgcacatcgacgccaggctcgaactgggtcaccaccaggaactggtcggcgaactccgctcgctgaccgccgcgaaccccctgcacgagccgcactggctgcagctgatgctcgccctctaccgctccggcaagcaggccgaggctctcgccgcctatatgcagctgcggcaggcgctggccgagaacctgggcatcgacccgggtcgtcagctccaggaactgcacctgcggatcctgcgcgccgacgcgggcctgctgacggggtccgggccggcggcaccggccgagccactgctcgtacggcagtcctga

SEQ ID NO: 27 - DNA, 420 bp, Streptomyces lavendulae
atgcgtggatcgaaggccctccgatacgcggcccccgtcctggtcgccgccgcaaccggcatcgccctcgccgcgggaccggcggccgccgtcccgatcggtcagtccgtgaacggcaagatgacctactacaccgaccagggctacggcgcctgcggcacccccatcgacgcgaactcccaggacctcgtcgcggtcccggccgcgtggtggacctccgccaaccccaacaacgaccagctctgccagggcatatcggtggaggtcagctacaacggcaggaccatcagagtgccggtgcgggacaagtgcccttcgtgcgaccggacccacatcgacctcagcaggacggccttccagaagctggcgccgctcgacaggggtgtggtcaacggcatcacctggaagttcgtccgctga

SEQ ID NO: 28 - DNA, 2811 bp, Streptomyces lavendulae
atgagttcatcaaatttaaggtcgcgggactcttggaacagatcaagacgacggagaacaatgacgtactcccccggcgcgcggccgcgcccggcccggctgtccgcactgctgctcgcaggcgcgctcgtcgcctcggtgccgcccgcggccgccgcgcgagcgccgcaaccccccaccgccgaccgcccccgcaccgccgcctcccccacaggcggctgccgtacgggtgacggctggacactcgactccacccgcatcgaccccgacgacacccaccacgcctatgtcggcaacggctacctggggcagcgcgtaccgcccaacggcgccggctacaccgacagcgacaccaagaccggctggccgctcttcgctccggcctacgacggctcgttcgtgtccgggctctacgcgcacaacaagcagaccgccgccgaccggcaggtgatcgccgctctgcccacctggaccggactggccgtcggcaccggcggcgagcacggcgatatcttcaactcttcgacgaagtcgggccggatttccggatatcaccagaccctcttccagagctgcggcatcgtccgtaccgccctgacctggaccgccgccgacggccgcaggaccgacctggtctacgaggtgctggccgaccgcgacgacccgcacacgggcgccgtacggctgagcatgacgccgcgctggagcggcgaggccaccgtcaccgaccagctggacggacgcggcgcgcggcgcatgcggcagaccggcggcggcgaccgcaccggtgggaccggccgggacggccgcaccatggacgtggccttccgcaccgacggcacggacaccgacggcgccgtcgcctccaccctgagggccgggcgcggtgtgcacacgaccggggaccgacgcgccgcggccgcgaaggacttgagcgtgaaccagtccctcacgttccccgtccgtgcgggccacgcgtacgaactcaccaaatacgtgggtgtcgacaccgcgctcacctcgcacgcgccccgcgaggacgccaccaccgcctccctgcgcgccgcccgccgcggctgggacgggctgctgcgtgcccacaccgccgcctgggcccggctgtggcgctccgacatcgagctgccgggacagcgcgacctccaggcgtgggtgcgttccgcccagtacgggctgctgtccagcacccggcagggggcatccaacagcatcgccccggccgggctgaccagcgacaactacgcgggcctggtgttctgggacgccgagacctggatgtacccggccctgctggccaccgcgccccaactcgccaggaccgtcgtcgactaccgctaccgcaccctcgccggagcgcgcgagaacgcccacaagctcggctaccaagggctcttctacccctggaacagcggcagcgagggcgacctggcccaggagtgccacagcgtcgacccgccccactgccgcacccagatccacctccagtcggacatctccctcgccacctggcagttctacctcgccaccggcgacaccgcctggctgcgcgagcgcggctggccggtgatggagggcatcgccgaattctgggccgggcgggtcacccccaacgccgacggcagctactccatcaaggacaccgccggccccgacgaatacagcaacggcgtcgacgacgcggtcttcaccaacgccggtgccgccaccgccctgcgcgacgccgcccgtgccgcgcggctgctgggcgagcgcgccccggcggagtggacgacgatcgccgaccggatccgcatcccgtacgacgcgcggcacaaggtcttcgagcagtacgacggctacccgggcagcaagatcaagcaggccgacacggtgctgctgatgtaccccctggagtggccgatgtcccaggccgacgcggcgcgcaccctcgactactacgcccggcgcaccgaccccgacggccccgccatgacggactcggtccacgccatcgacgccgcggccacgggcgagccgggctgctcggcgtacacctatctccagcgttccgtccggcccttcgtgcgcggtcctttcgaccagttctcggaagcccgcggcaccaaggccggcgccgacgaccccctggccggctcgcccgcccacgacttcctcaccggcaagggcggcttcctccagatcttcaccaacggcctgaccggcatgcggatgcgcgaggaccggctgcacctcgacccgatgctgcccccgcagctcggccgcggcgtcaccctgcgcggcctgcactggcagggccgcacgtacgacatcgccatcggcgcccacgagaccaccgtgcggctcaccgggggtgcgcccatgaccctctacaccccgcagggcgagcacgtgctgaccaaggcggcaccggccgtgctcaagacccgccgccccgacctcgctcccaccgacaacgtggcccgctgcaccaccgccggtgcctcctccgaggaacccggtatgtacgcggcagccgcggtcgacggcaaccccgccaccgcctgggtccccgacgggccgaacggtgaactgaccaccgacctcggcaagtccgtacgcgtcaccaaggccacccccgtctggagcggcccggcaccggcctcgtacagcgtccagctctccctcgacggccggcactggcacgacgcggtcgcgggcggcgctccggtgtccgcgcggtacgtacgcgtcgcgctacgcggtcaggccgatgccaagtcccgtacgggcatcgccgagctgaccgttacgtag

SEQ ID NO: 29 - DNA, 813 bp, Streptomyces lavendulae
atggaattcctcgggccggcggccggtgtctcgggcgccacgcggctgtacgcggtgctgggtgatcccgtcgcccaggtcaaggcgcccggtctgctcaaccccctgctgagcgaaagcggtctggacgccgtggtggtgccggtgcacgtccgggcgcgggatctcgccgaggtggtcgaggggctcaagcggatcggcaatctggacggtctgctggtcaccgtgccgcacaaggcggccctgtgcgggctcgcggacgggctcgggccggcggccgccctcatcgggacggccaacgcgatgcggcgcgaacccgacggccgctggtacgccgagaacttcgacgggctcgggttcgtccagggtcttcaggcggccgggcacacggtgcgcgacaggcatgtggcactggtcggcgccggaggggcgggcagcgcgatcgccacggcgctgctgatggccgacgccgcgcgggtgtccgtgcacgacaccgaccgcgcccagctcgacgcgctgctgctgcggctcgggtcccgccggccggacgggatccgggcgctggggcccggcgatctggaggcggccgatttcgccgtcaacgcgacgcctctgggcatgcgttccgaggacccgctgcccttcgaccccgcgagggtgcgaccggatgccgtggtggtcgacgtcgtcatgaagccgcacgagacggcgctgctgagcgcggccgccaccgccgggcgccgtgtgcaccacggcatccatatgctggagcagcaggttccgtgctaccgcgcgttcttcgggtggccgtga

SEQ ID NO: 30 - DNA, 948 bp, Streptomyces lavendulae
gtgacgggggacaccgacggtgcgggcggcggcgacgtgacgttccgctggcccgccgccggcgacgtcaccgcggatctggacctgctcgccgcgcgggtccgcggtcttctgggacaccgcgaggaccccctcgccggggtcggcgtggccatgcccgcgatctgcgacgcggccgggacggtccgcacgtggccgggacggccgagctgggcgggcctgaacctgacggccgccttcgggcagttgctgcccggcaccccggtcgcctgcgccgacgacggtgacctggccgcgctggcggagtcccgcgccgccggctgccggcatctgctgtacgtgggggtcggcacgggcatcggcggcggcatcgtccatgagggccgcgcctggccgggccccggacgcggctcgtgcgaggtcggccatgtcgtcgtcgaccgctcgggcccacgctgcgactgcgggcgcgccggctgcgtccaggcggtcgcgtcgggaccggcgaccctccggcgggccgccgaacggcgcggccgggagaccggcttcgacgaactggcctccggggcgcgcttgcacgccccgtgggcggaagcggccgtcgacgagagcgccgcggccctggccaccgccgtgaccggcatctgcgagctggcccaccccgaactcgtcctcgtcggcggcgggttcgcggcgggcgtgccgggatacgtggcctcggtggcggcgcacgtcgagcggctgacccgcccgggaacggatcccgtgcgggtgcgcccggcggtgctcggcgggcggtcctccctgcacggcgcactgctgctcgcgcgggaggcacacgggcggggaaaccggccgccggagagtgaccgtgtttcttccgatgtttcttccgatgtttctttcgggggagtgacagacagggccgttggccggtccgactga

SEQ ID NO: 31 - DNA, 1545 bp, Streptomyces lavendulae
atgctcgacaggcggagcgtcattcgcgtcggcgccggggtggcggcggccgccgccgtggccggtacggccgccaccggtgcggcggccgtggggctgccgggtgtacggggacgcgcggcgtcgcgcggggtcgactgggcctccttacgccgtcatctgtcgggcgagctcgtcctgccggcggacaccggatacgagcgggccaggaagctctacagcggccagttcgacggcatccgcccgcaggccgtcgcctactgccggaccgaggaggacgtgcggacgaccctcgcgttcgcccaggaccacgcgctgcccctcaccccgcgcagtggcgggcacagcttcggcggctactccacgaccgacggaatcgtcctggacgtctccggcttccacgcggtgagcctcacccggaacaccgtcgtcatgggcgcgggcacccagcaggtggacgccctcaccgccctgtcgccgcgcggtgtcgccgtggcgagcggcaactgcgcgggcgtctgtcccggcggcttcgtccagggcggcggactgggctggcagagccgcaagttcggcatggcgtgcgaccggctcgtctccgcccgggtcgtgctcgccgacggccgcgccgtgaccgcctccgccaccgaacaccccgaccttttctgggcgatgcgcggcggaggcggcggcaacttcggcgtcgtcaccggcttcgagctgcgccccaccgacgtcccctccgtcgtcagctacaacctcacctggccgtgggagtcggcgcggcgcgtcatcgaggcgtggcagcactggatcatcgacggcccccgcgacctcggtgccgcgatggccgtgcagtggcccgacgccgggaccggcacgccggtcgtggtcgtcaccggcgcctggctgggcgcggccgacgcgctcacccccgtgctggactccctggtggcctccgtgggcagcgcgcccgccacccgctcggccaaggcgctctcccagcacgacgcgatgatggcgcagtacggctgcgccgacctcacgcccgagcagtgccacacggtcggctactcgcccgaggccgcgctgccccggcagaacttctccatggaccgcaaccggctcttctcccgggccatcgggcaaggaggcgtcgagcggatcctggaggcgttcgccgccgacccgcgcgccggacagttccgcttcctgagcttcttcgccctcggcggcgccgccaaccgccccgaccgcaccaccaccgcctacgttcaccgcgacaccgagttctacctcggtttctcgatcgggctgaacgacccggagtacacggcggaggacgagaggctcggccgcgcctgggccgcgcgaggactgcgcacgctcgatccccactccaacggcgagagctaccagaacttcatcgacccggagctcgacgactggaagtcggcctactacgccgagaactacgtgcgcctggccgccgtcaaggcggcctacgacccgcaccggctcttctccttcgcgcaggccgtctga

SEQ ID NO: 32 - DNA, 495 bp, Streptomyces lavendulae
gtggagagggtggagctgatccgctggccggtggagtccgagcggcgggagcgctgccgcgaccggggcgtcatgcggatcctggtgctggaggcgggggccgaggcacccttgtgcgtggaccccaaggaggactgggtccgcgctcccgtcagcaccgacgacctgcgggcccgcgtcgaggccctgcgccttcggggagccgccgccgagtcccggcccgaggtcgacccgaacggagtgctgcgtttccggtggcgctccgccctgctctcgcccaccgaggcccggctcgtcgcccggctcgccgagtcctatgccgaggtcgtcgcccgcgacgacctgctccgcccgcccccgggccgtaccgtgccgagccgtaacgcgctcgacctccacatcatgcggatccgacggcgcctcgccgcgctgggcctgagggtgcgcaccgtccgggggcgtggctacgtcctggagagcgcggaaggagtctga

SEQ ID NO: 33 - DNA, 1032 bp, Streptomyces lavendulae
gtgcagcagcctcatcacagccgcgtcgacgtggaactgggcgagaggtcctaccccgtccacgtcggaccgggggtccgccacctcctgcccggcatcgtcgcctccctcggcgcgcaccgcgccgccgtcgtgaccgcacggccccccgacctggtgcccgatcccggcgtgcccgcgctgatcgtgcgggcacgtgacggcgagcggcacaagacgctcgccaccgtcgaggacctgtgccgcaagttcaccaccttcggcatcacgcgccacgacgtcgtcgtctcctgcggaggaggctcgacgaccgacaccgtcggcctggcggcggcgctgcaccaccgtggggtgccggtggtgcacctgccgaccaccctcctggcccaggtggacgcgagcgtcggcggcaagacggcggtcaacctgcccgagggcaagaacctcgtcggcgcctactggcagcccaaggccgtgctgtgcgacaccacgtatctccagacgctgcccgccgaggagtgggtcaacggctacggcgagatagcgcgctgccacttcatcggtgccggcgacctccgcggcctcgccgtccacgaccaggtcaccgcgagcctgcggctgaaggcgtccgtcgtcgcggccgacgagcgggacaccggcctgcggcacatcctcaactacggccatacgctgggccacgcactggagaccgccaccggcttcgggctgcggcacggactcggcgtggcgatcgggacggtcttcgcgggccggctcgcggaggcgctgggccgcatcggcgccgaccgcgcgcgggagcacaccgaggtcgtccgccactacggacttcccgacagcctcccgggaaacaccgacatcaccgagctcgtcgcgctgatgaggcacgacaagaaggccacgtcgggactgaccttcgtgctcgacgggccttccggcgtggagctggtgtccgggatcccggaggacgtcgtcctgcgtacgctcgcggcgatgccgcgaggaacggcctga

SEQ ID NO: 34 - DNA, 441 bp, Streptomyces lavendulae
gtgttccgtcttccgaggggaagtgaccgtttcgtgtcggcagagctgtcagaaccgctgaagaaggccctggactccctggtgttcggcgtcgtggcgacgaccgaccccgacggccgcccgcaccagtcggtggtgtgggtccggcgcgagggctccgacgtgctgttctcgatcacgcgcggcagccgcaaggagaggaacatcctgcgcgacccgcgtgtgagcgtgctgatcagcccggcggactcgccgtacacctacgccgcgatccggggcaccgcgcacttcgaggacgtgccggacccgggcgcgtacctcgacacgttctccataaagtaccacggcgtgccctaccgggagtcgttccccgagccgccggaggtgagcaccattctcgccgtccggctcgttccgacgtcggtctacgagcagtggtga

SEQ ID NO: 35 - DNA, 828 bp, Streptomyces lavendulae
atgacggaaaccgcgtccgcctccgaccggatggtcgaactctacaaccgcgtcaccgacttgatggtgcacgcggaaggcggctacatgcacggtggctactgggcgggacccgacgtccccacgacggtggaagaggcaggcgaccggctgaccgactacgtctcggagcgcctgcgcctcgcccccggggagcgggtgctcgacgtggggtcgggcaacggcaaggccaccttgcgcatcgccgcccggcacggggtgcgggccaccggggtctccatcaacccctaccaggtgggtctgtcgcggcagctcgccgagaaggagggcgacgaggcgaccgagttccgcatcggtgacatgctcgcgctcccctttcccgacggctcgttcgacgcctgttacgcgatcgagagcatctgccacgccctggaacgggccgacgtcttcaccgagatcgcccgggtgctgcgcccgggcggccgggtgacggtgacggacttcgtgctgcgccggcccctgagcgacgcgtccaggacgatcgtcgacaccgccaacgacaacttccagcagggccccgtcctcacccgcgaggcgtacgaggactgcatgcggtcggtggggctggaggtggtggagttcctcgacatcggggacgaggtgcggccctcctacgaggcggtggcggcgaagatgcgtgcggccagggacgagctcggctcccacatggacgacgaggcgttccaccgcatggtcgacggcatcgaccgcatgggctcggtggaggaggtcggctactcggtggtcaccgcgcggaaaccggcgtag

SEQ ID NO: 36 - DNA, 852 bp, Streptomyces lavendulae
atgccgcactccgagctgtccgaactccccatgccctcacccgcctccgaggaagtgggcgcgctctacgaccggttcaccgcgctgggagccgcctccctcggcgagaacctgcacttcggctactgggactcccccgacagccaggtgccgctggccgaggccaccgaccggctcaccgacatgatggccgagcggctgcgcatcggcgccggctcccgcgtcctggacctcggctgcggcgtggggaccccgggcgtacgcatcgcccggctcagcggagcgcatgtcacgggcatctcggtgagccatgagcaggtcgtccgggccaacgcgctggccgaggaggccgggctcgccgaccgggcgcgcttccagcgggccgacgcgatggacctccccttcgaggacgagagcttcgacgccgtcatcgccctcgaatcgatcatccacatgcccgaccgcgcccaggtgctcgcccaggtcggccgggtgctgcggcccgggggccgtctggtgctcaccgacttcttcgagcgggcccccctcgcccccgaggggcgggccgccgtccagcgctacctccacgacttcatgatgaccatggtcagcgccgaggcgtaccctcccctgctgcggggggcgggcctgtggctggaggagttcctcgacatcagcgaccagaccctggagaagaccttcaggctgctctcggagcgcatcaactcctcgaagcagaggctggagacgcagttcggcgaggagatggtgaaccagttcgaccccggcgacctcgtcggcgtcaaggagttcggctatctgctgctggtcgcccagcgcccgggaaagtga

SEQ ID NO: 37 - DNA, 1563 bp, Streptomyces lavendulae
gtgctgaacaccctgtccaccgcgccgttcctgtccacggcctggctcgccggggccgcgaggctcgaacgcccgcccgtgggcgaacgcggcacggtcgcgctccgcctggagctcaccgacccaccgcccggcgaacccccggccgtcgacgtccaggtggacctcgtcgccgggcggctcggcctcgcggccgcggccggtgagagtccgggactgcggatccggcttcccctggaggccgcccgcgccctgctgctcggccccgcgcgggatcggaccggcgtattcgagcggggcgacgtacgggccgagggcaatttcagcctgctgttcttcatcgacgccgcactggagcgggacgcctcgggccatgtggccgcgctcaggggcacgcccggtaccacggcgcgggaagcggccccgccgcccggcaccgaggacgcggccgaggccgtccggcgcgcccgtgcggcgcttcccggcaccatgcgggagctggagcgcgaggtcggcacctcgaccccgggggcgcagatctacgtctcccgcgacggagtccctctggcggacgccgggttggggctggcccgccccggggtggcgatgacccaccggtcgctgcccctgtggtactgctgcgccaagccactgctgtcggtcgccctgggccggctgtgggaggcgggagcgtacgacccgtatctgcccgtcgcgcactatctgccggagttcggcaaccggggcaaggagtccatcacctcgatggaactgctgacgcatacgggcccgctgcccaccggcgacgacccgctgcacggcatcgtggccggcccggacgaggagcgtgtgcgccgtgccttcgaggtgccggtggcaccgcgtccggggggcacgcccggcatcaactacagccagtggtgggcctggttcgtcctggcgcgcatccttccggtcgtcgacggcagggagtaccgcgcgtacgtccaggaggagatcctcgggccgtgcggcatgtccggcacccgtgtccacctggatcgcgaggagttcgccgcgctcgggggcgagctgccgctgatccatgtgagcaaccccgagggcggcccgctgcccacccactggtggtcgacggaggcggccaccacacgctgcatcccgggggtcaacacccgtggcccgctgcgggacatgggcaggctcttcgagatgctgctgcgcggcggggacgctcccggcgggcgcgtcctggcgccgcccaccgtcgccgccctcacggcccggcaccgcaccggcctccaggaccgctacggcaacgccgactggggcatggggttccgcctcgaatgccgtcagctggatccgcggttcaccagcttcggctcgtacgcctccccccggtccttcgggcacgacgggctgtggaccgccgtggtcttcgccgacccggacgccgctctcgtcgtcgccctccacctcaacgggaaggtggagcacgaacggcaccgcgagcgcatcgtccgcctcgccgacgccgtctaccaggacctccgtctctcctga

SEQ ID NO: 38 - DNA, 1041 bp, Streptomyces lavendulae
atgacgcccgcgaccccgcgctggagcgtcgtcgccccgcagggcaccaacctcgaactggccggtacgggcggccgcgagggctggcggctcctcctggagaccgcccgcaccgtccaccggcacggccgtggcgccctctggctgctggaccgcaccgacaccctgccccggcgcgagcccgagccggtctgggagggctggacggcgctggcggccctcgcgggcgcggtgcccggtctggatctgggactgctctcctcggccccgccgttccgcaacgccgcgctgatcgccaagcgggccgcgaccctggacgtcgtctgcgacggccggctcaccctcggcttcccggcccgcgagtacctgccggagcaccactcgacggggcgcgaggtgcccacgggcctggaggcggacgaggaggaggccgccggccaccgggctctcggcgagacggtcgaggccctgcgcgcgctgtggggcggacagcccgtcaccttcaccggggaacacatccgcctcacttcggcgcactgcgtgcccgccccacggcagcagcccctccccctcgcgctgcgcaccccggccggggacgccgggagcggcgcgctgcggcccgccgacgccaccgtgcgggagtgcgctcatgtccagtggaccggtgagcccgctcaggtcgccgcggccgtcaccgcgttccgccgccgtcgcacggagctcgggctcgatccggacggcgtccggcacgcctgggccgcggagtgccggatcttcgactccgtcctggaacgcgaccgctggctctccaccccgcacgaggtgctgttctggagccaccatcccgacctgctggcgcggcgcagcctgtacgggacgccggaacagctcaccgagcgcgcccggcgcctggtcgccgcgggcgtggcggagttcgtgctgtggttccgcgactacccggccaccaccagcctggagcggctgttccaggaggtcgtcccccaggtggcgccgggggccgccaaggaagcggaggagtga

SEQ ID NO: 39 - DNA, 708 bp, Streptomyces lavendulae
atgcccctgaaccccccgcccgcctcgcgggccgcggcggacgcgcccgccaccgctctcccgtgccggttcaccaccgtggtcttcgacctcgacggcgtcctcatcgacagtttcgcggtgatgcgcgaggcgttcgccgtggcctaccgcgaagtggtggggccgggcgagccgcccttcgaggagtaccgcacgcaccagggccgctacttcccggacatcatgcggctgatgggcctgcccggcgagatggaggaaccgttcgtccgggagagccaccggctgatggaccgcgtcgaggtgtacccggacgtgccgcagttgctggcggagctgcgcgcggacggcgtcggcaccgcgatcgccaccggcaagtccggctcccgggcgcgcgccgtgctggaggcggtcgggctgctgcccctgctggacgaggtggtgggcagcgacgaggtgccgaggcccaagccgcaccccgacatcgtgcgggaggcactgcgccggctggacgcggcgcccgaggacgcggtcatggtcggcgacgcggtgatcgacatccgcagcggccgcgccgccgggaccgccaccgtgggcgcgacctggggcgagggcgcggccggccaactgcgcgccgagcggcccgacttcctgctggacaagccgcagagcctgctcgcgctggtccgcagcggcggccacgcatga

SEQ ID NO: 40 - DNA, 873 bp, Streptomyces lavendulae
gtggagcgtctgaagctcgtgcccgacgagcaccgccgtttcaccgtcgacgagcagagcgcgcgccggctgcaccggatcggaccggagctgctgtccgcgctgtgcgaggcgggcgtgcccttcgtgggaagcggcgcgggacgcctcttcgacggctacgacctgggcaacgcggccctgcaccttggcctgtcctcggtgcagcgccgggccatccgctcgtgggccggttccctgcggaccgcctcggccgcggagagcccgcgctggcgcgtcgacgtcacggcgtcctgcccggtgcccggccacgcgggcccgtgccgctacggagtgctgctgcccggcgcccgccgcccggtggaggcggcttcgccgcgggagaccacgctggcgcggctgtacacacggtcgcgcggccactggccggacttccccccggccgtcctcgacctgctgcgcaccctggagccggtcggcttcttcctgctgcccgaagcgatccgctgggacccggggttcctgtggagcacgcacatggccgactgcggcggcgccgcggcctggctggtggcggagggccggcggcgcgggctcgacgtgcggttctccttcgggctgctggtggccaagccgtactccacaccgcactgctgggccgagttcctggtgggcggccgctgggtgccggccgatccgctgctgctgagggccatggccgcctggggcgggctggacgcggcggcccacccgccgcacagctcgccgggggccgtctaccaccggctcgcgggccgcttcacgaaagtcgtcagccacgccggggtctgggccccgacgtccctacccacggagctcctgccatgcccctga

SEQ ID NO: 41 - DNA, 1149 bp, Streptomyces lavendulae
atgaaattcgcttatttctcccatgtctggggacgtcccggtatcacgccgggcgagcgctacgaagagctgtggcgcgaggtcgaggacgccgaccggctcggcttcgactacgcgttctcggtggagcaccactgcacgccgcacgagagctggatgccctcgcccgcggtcttctgcacgggcgccgcgctgcgcaccgagcgcatccgggtcggcccgatggggtgggtgccgccgctgcgccacccgctgcacctggtcgaggaggtcgcgaccctggaccagctcctgggcgggcggctggaggtggggctcgcctcgggcgtcagccgtgaccccttcctgcccttcgacgccgatttcgacaaccgtcacctcctgacccgggaggccctggagctgctgcgtgccgcgttcgccgcgcggggcgccttcgacttcgacgggcccgcgcaccggctgcgcgacatcgccctgtccttcccgccggtgcagcgcccgcacccgccgatgtgggtgcccaccaccaaccgcaacaccttgcgctatctcagcgaggccggtgcccacaccagttccacgatgatcgtgccgcgcgcctccatggcgctggtctaccggcactacctcgactggtggcgcggccacggccacgcgagcgacccgcgcatcggctactggacgctggtccacgtggcccggacggacgccgaggcggaggagcgggcggccgcgcacatcaccgagacgttcaccaagacgctgcggtacggctcggtgtcccgttcccgcgatcagcacgccccacccagcaggctcagcacgacggacatcctggcgggctccggcgacctgcgcttcctgctggagaacaacctcgtcttcgtcggctcgccggcgaccgtggccgaccggatcagggccgcgtccctggagggccatttcgacacgctgctgggcgagttcaccttcggcgagctggcggaccggcaccgcatcgagtccatggaactgttcgcgcacgaggtggccccggcactgcgcgccttctccccctacgcgccgcgcccgcaggagccggcgtacaccgcgagcgacgagcagcaggtggcggcccgcctccaggctctgggctacatcgactga

SEQ ID NO: 42 - DNA, 1215 bp, Streptomyces lavendulae
gtgacaccgccgacgacagctcgcgaacccctccggatggcagtgctgggcgcgggatgggtctcgcgcaaggtgtggctgccgctgctggcggaacacccggcgttccgggtcgacttcctcgtggacgacgaccccgtggcggccaggtcggccctgccggagggcgcgcggacccgcgtcctgagcagaccggaagagctcgcccccagaagcgtggacgcggccatcatcgccctgcccaaccacctccatctccccgtggccaaggccctcctggagcgggacgtgccggtgttcgtcgagaagccggtgtgccgcacgctcttcgaggcccaggcgctcgccctggaccaccaggcgcggggcgacagcatcggggacatcaccctctacgcctggagcgccgcccggcaccgcaccgatgtctgccgcctggcggagctgctgccctcgctgggcaccgtgcgcagtgtcgggctgagctggatccgggccaccggcatcccgcagcgcaccgggtggttcgtcgaccgccggctcgccgggggcggcgcgctgctcgacctgggctggcacctgctggacgtgggcctgcacctgctggggtggccgcgcgtggtccgggcggcgagcacgatgtccgcggactggatgagccggggcgaggccacggccgactggagccggcgctcctccggcacggcgcggccaggccccggggagacggtggaggacaccgcccgcggcttcctcgtcaccgacaccgacgtgggcatctccctggagacacgctgggcctcccaccaggcgctggacgtcaccacgatcaccgtggagggcaccgagggggtggcgacgctgcgcggcaccttcggcttcagcccccaccggctacagaagtcgagcctcgtggtcctgcgccagggggtggaggagaccgtcgcgctgcccgacgagcccgtgggcgtggagtaccggcggcaggtggacgaactcgcccgcaggctcggcggctcggccgacgggcagggcccggtgtcgggcctgggcgaggggtcgatggccgaagtgaccatcctggcctcctgcatcgaccacatctattcggccgccggcgtcgaccctccctcgcccctgcaccggccgcagagcgacgcggcgcccagcacgtccagttgtccacgtgtcctgcccacccggggaagccaatga

SEQ ID NO: 43 - DNA, 774 bp, Streptomyces lavendulae
atgagcaccgtcaccgaccgggccacggagcgcctgggacagagcggccgcgtggtcgtggtctcgggcgcgtccgggcagataggcggcgcgtgcgcgctggagctcgccgcgctcggcgccaccgtcgtcgccggctaccacagcggcgagcaggcgatccgcaagctgcgggagcaggtggagggccagggcggcaccctcgtgcccgtggcggcggacctgagcgaacccgagggcgccgacgcgctggtggcggcggccgtcgaacggttcgggcgggtggacggctgtgtggctgctgcgggcttgcgtacgcgccggctcgcgatggccacggacgcccggagcctggagaagctgctgcgggtcaacctggccggttccgtgggtctcgccaaggcgtgcctgaagccgatgatgcgcgccaggtacgggcggatcgtgctcttcggctcccgggccgggaccagcgggctgcccggccacagcgcgtacgccgccaccaagggggcgctccagccgtgggcggcgtcggtggcgggtgaggtcggcaagcacggcatcaccgtcaacgtcgtcgcgcccggggcgatccgcgccgaggtgatggacttctcggaggccgagcgcgatctggtcctgcagttcatcggggcggggcggctcggtgagccggaggaggtcgcggcggcggtgtcgttcctgctgtcgccgtcggcctcgtacgtcaacggcaatacgctcgtcgtcgacggtggtgcccgcttctga

SEQ ID NO: 44 - DNA, 2124 bp, Streptomyces lavendulae
gtggccctgcgcgcgcccaacagcccgcggtgggtcgtcgccttcctctccctgctggcatccggcgccaggcccctgctgctcgaacccgacacccccggccccgagaccgcgcggctgctgcgggctgccgggggcggcaggtccctggtcgtccccgggaccggcgacggcctccgcctgacgttgaccggctcgcccggagaaccctccggcgccccgcccgccgtgctgctcccgacctcggggtcgaccggtgcgagcaagctcgtcgcccgcagcgaggagagcctgctcgcggagggccgccgctaccgcgacggggtcgggctgacgggagaggacaccctgctgctgccggtgccgctgtcccacgcgtacgcgctgggctggctgttcggcggactgctgacgggtgccgcgctgcgccccgtaccgccgaccgccctcggccgcatcgccgcggagctgtccggtggtgcgaccgtggtggccctggtgcccagtgtggcccggctgctggcgacccggcggctgcggggagcagcggccgggcgggcgcccgccgctcccggtctccggctggccatggtgggtgcggggccggtggacgagcagctggaccgcgcgttcaccgaggcgttcgggaccggtctcgcccgcaactacggttccacggagacgggcgccgtgctcgccggaccggcggggctggagcccttgtgcgccggtgctcccctgccgggggtggagtgcgaactgaccggcccggagggcgtggtgccgcccgccggcaccccggggctgctgagcgtacgggtcgacggccggccgtacgccatgggcgatctcgccgtggccgtgcccgggggcctgcgcatcctgggacgcgaggaccgggcgatccgccggggcgggcgctgggtctccccgctggagatcgaggaggtgctgcgcggtcatccggacgtggtgaatgtgcgggtgggcgcccggcgggggcggcaccggggcgaggacgggatcgtcgcggaggtctcggcggcggggccggggctcacccccgaggcgctgcgcgagcacgcccgccgggagctggccccgcacaaggtgcccgacgagttcgtcctgcgggagagcctgccggtcaacgccgcgggcaaggtgcgggcggcgtccgtctaccgcctcacccggagcgcggcggaggccgcccgggcgtacaaggcatccgaagtgctcttcgcgctgcacgacttgggcgccctggaggcactcgcccagggtgccggcacggctctcctcgccggggagctggggtgcgacgcggatgccctggagtggctgctgcgcacggccaccgctctgggggtgctgaccaccggggcgcaagagcccggggaccgggtccgggccggggagctggccgcgttcgtggcgctggaggagcacctctcccgtgggctggtcacgcgcgaggagctcgtcgcggtggcccggagcgggacggcgcggcgtcccttcgaggagcgtccccccgagagcctcggtccgctcgtcgccctgtaccagggcgcgatggacggccccggcgcacgggcccgggccgcgctcggcctgcggctcctgcggcccgggccgggagcccgggtggtggaggtgaccgcgggcccgggccgctatctggaacgcctgctcgcctcggaccccggggcgagcggccatctggtcaccgtcggccggctgagcgggccgctctcctcggccgtcgccgcggcggtcgaggagggcagggtgaccgtggggacggaactgcccgtcggctacgccgacttctgcgtggtcgccaacgccgtgcacggcccggggccgggcagcgctctcggtgccctgctcggctccctgcggccgggcgggcggctgctggtcgacgacgtcttcctgccggcgtccgggccggggagcgaactggctctggactggctcacgcacggcgggaccgcgtggccggccaccggcgagctgatcgccgggctgctgcaagagggggcggaggtcgcacggcacgtgccgctggacgcgtccccctgtcatctgatcatcgccaaggaggccggttcatga

SEQ ID NO: 45 - DNA, 1152 bp, Streptomyces lavendulae
atgtcccgtagcacccacccgccgacagccacccccgacgcgggcaccaggcgacgcctgccgctgatcggcaacgacctggtcatcaacgaggactcctgcaacctcagctgcacctactgcctcaccggacagagcaacctcaaggagggccactcccttcaactgatcttcgagcccccgcggcgcgacagctacgccaaggacagcgggctggggcagcgcatggacaaggtcgccgaccggatccgggaccgcttcggcctgccgctgctcaaggtgaccggaggcgagatcttcctggtccgggggatcatggacttcctggagcaggaggcccgtaaatacgacgtgctggtcatccagaccaacggtgtcctggtgcgcgaggagcacctggagcggttccgctcgtggggcaacgtcgtgctccaggtctccctcgacagccacctccaccacggcaacagccatcgtgtgccgtccgggagcctgcacgagaaggtcgtcgccgccatcgcccggatcctggactcggggctgccggtggagatctattcagtgctcaacgaccggagcgtcacggaggtctgcgcgttcgccgagtggctgtcgggattctcccggcctcccgtctacttccccttcccggtgcggggcccggactcggaggacttcaaggtgcggcccgggcagttcggccacatccaggaactcgtcgaccgctacgacgagttcgcgcgggtcctcccgccgcggccctacttcgaccggctgacgagcttctaccgcgagggccgccgcaccttccgctgccatctgccgcggctggtcgtctccagcttcagcgacggcgtcgtcacgccctgccccaacatctggttctccgacatgggcaacgccctggaggacgactggagcgagatgctggacacggtgggcaccagcggcctctaccgtgccctgctcgcccccaagccccggctcaaggcgtgccacggctgcttcacgccctgggacacgctctcgatgtacttcgaggacgagatcaccctcgacgagctgtgcgccgctcccacctactccccgccccgcatccggcagatgctcagcgacgcgaaggccgactacctccagggcggccatgactga

SEQ ID NO: 46 - DNA, 21 bp, Streptomyces lavendulae
ggcaaggcatgcgagggtcgc

SEQ ID NO: 47 - DNA, 30 bp, Artificial Sequence
  A primer
ttccagaacggcgccctgatgaccgccggc

SEQ ID NO: 48 - DNA, 30 bp, Artificial Sequence
  A primer
gccggcggtcatcagggcgccgttctggaa

SEQ ID NO: 49 - DNA, 1545 bp, Streptomyces lavendulae
gtgccaccctctccccgcgccctcgtcatcggaatcgacggaggcacattcgatacggtcgacccgctgatcgagtgcggtctgctgccccatatggcgaagttgctgcgcgagagcgccagtgccgccacggactgcacctggcccgcccacacggcgccggggtggagcacgttcgtctccgccagcgatcccggcggtcacgggatctatcagttctacgacacccaggacccggcctacggggcccgcgtcacgcgctccggcgacctgggccggtcctgcgcctgggactggctcgccgcgcaggaatattcgctgggcctcatcaacatcccgatgtcgcacccgccggccgacctccccggctatcaggtcacctggccgctggagcggacactcaagcactgccgcccggattccctgctgcgcgaactcgccgcggccaaggcccatttccagtcggacctcgcgaccatgttccggggcgacatggcctatctggaggaggccgagcgcaatgtggcggcgcgggtccgctccgtacggcatctgatgagcacccggcccaccgatgtcgtgatggtcgtgctcaccgaggccgaccgggtcggccaccactactggcactacggcgaccccggtcacccgggccaccggcccgccccggagggcagcggctgggacgtcgccatgccccggatctaccaggccatcgaccacgcggtgggcgagctcctggagctcgtggacgaggacacctccgtcgtgctcgtctccgaccacggcctgggcaccgggcgccacggcctgtcggtgcacaccctcctggaggaggccgggctgctggccaccgcaccgggggaggagccgcaggacgcggcggcgagctggttcgcgggcaacggccggcacgtcgacttccgccgcaccagcgtctacatgcccgtccccggcagctacggcctcaacatcaacgtacgcggacgccagcagcgcggcaccgtcgcaccccgcgaccgcgaacgcgtcatggacgaggtcacgggcctgctctccgggctgaccggccccgagggacagcaggtcttccgggccgtccgcccgcgcgaagaggcgtacccagggccgcacaccggccgggcacccgacctcctcctcgtcccgcgggacgagaccgtcctgcccgtccccgacctcggcggtgacgtgtggcggccgagcgcgcagaccggcctgcaccgctaccgcggcctgtgggcgcaccgctcgccccgcgtccgccccggccgcctgcccggcaccgtcgcgctcaccgacaccctgcccaccctgctcaccgacctcggggccgcatggcccagcgacatccacggccgccccgtgaccgccgtcctcgacgacggcgtacgcgtcccgccctccgacccccgggtcgaggccaccggcaccccggccaccacgatcccggccgccgcttcggccgctgatgccgccgaggacgcgtacaccagcgaccgcttgcgcgaaatgggctacctgtaa

SEQ ID NO: 50 - DNA, 282 bp, Streptomyces lavendulae
atggagaccctgacgaccgacaagatcaaggaccggctgcgcaaggtgctcgtcgattccctcgaactgtccctggacccctcggccgtacccgacgagggactcgtggagaagctgggcctggactcgatcaacaccatcgaattcctcatctgggtcgagagcgaattcggcatagagatcgccgacgaggacctgtcgatcaagctcatcgacagtctcgacctcctcgccggctatgtgtccgagcgcgtgaacggcgtcaccgcacccgccgaatga

SEQ ID NO: 51 - DNA, 1413 bp, Streptomyces lavendulae
atggaccggcacgccctggtgatcgggctcgacggcatgccgaggaccctgctgacccgcctggccggcgacgggaccatgccgcacaccgcggcgctgctcgccgagggccactgcgcggaactgctggcacccgtaccggagatcagctccacctcctgggccaccttcctcaccggcaccaacccgggccggcacggcatctacggcttcaccgacctcgcccccggcgacggctaccgcatcaccttccccggtgtgcggcagctgcgcgaacccccgctgtgggaactcgccgcccgcgccggccgcaggaccgtgtgcctgaacgtgccgggcacctaccccgcccccgccatcgacggcgtgctggtctccggcttcgtcgcgcccgaactggagcgcgccgtcagcccgccacggctgctgccgctgctgcgcggcctcgactacgaactcgacgtcgaggtcggcgacgtcgccgccgacccggccgccttcctcgggcgggccgtccgggccctgcgcgcccgcacccgggcgatggaacacctgctgcgccaggagacctgggacctcgcggtcgccgtgctcaccgagaccgaccgcgtccaccacttcctgtggcgcgcggtcgccgaccccgccgaccccctccacggggacgtcctcgccttctaccgcctcgtggacgactgcgtcgccaccctggtgagcaccctcccaccgggcggcgaactcttcctgatgagcgaccacggcttcggacccgccgcctgtcaggtctatctgaacgcgtggctcagggagtccggctggctggccgggctcgacgtctgtccggacctcaccgcggtcgacgctcgcagcaccgccttcgcgctcgaccccgcccgcatccacctcaaccgcaagagccgcttccccggcggcggcctgaccgacgcggaggcggacgaggccgcccacgagatcgcgcgcgagctgtccgccctgcgctgcgacggcacccgcctgggccccgacgtcgacggacccctgctcgtccgcgacctctaccgcgctcaggagatctaccacggcccgctgttgggcaacgcccccgacctggtggccgtaccggcccccggggtgcagctgcgcggcggctggggcggcacgcacaccgtacgcaacgacatcctcaccggcacccacacccgcgacgacgcggtcttctaccggcgcggcgcgcccgcgcccgcccccggggcggacgacggccccctcgacatgacggacgtcgccccgaccgtcctcgcctccctgggcatccaccccggcgggctcgacggcgcggccgtactcggcaccacgggacccgcgtccggtcacggccgcacggacccccctctcgacatcagggagctctga

SEQ ID NO: 52 - DNA, 1836 bp, Streptomyces lavendulae
atgaagcacgacctcggtctggcaccatcggcacccaaaccgggaacactcgacctgagcctggacccacgcatcacggaccccgcttccttccgggtcagtttcctgatcctcctcgacggcgacctcgtgatgtcccccgaacacctcggcgtcgcctacatggccggtgtgctgcgccatacgggcttcaccgcggagatccgggaggtggagcacggcgacgaccaggcggccgccaccgtcgaggcgctcaaggagtaccggcccgacctcgtctgcttcaccctgatgagcctgaacctgggcagctgtctgaccctgtgccggatgctgcgggaggagctgccggggacgacgatcgcctgcggcggcccagccgggaccttcgcgggcctggacgtcctgcggaacaacccctggaccgacgtcgtcgccgtgggggagggcgagcccaccatcctcgacctcgtccaacggctctacctcaaggagccgttgtccgcctgcaaggggatctgctaccgcgacgaggacggcacaccgcgccagaaccccgcccgccccctgatccacaacctggaggacctccccttccccgcccgggaccagctgcgccagcacggcgacaagctggagtacgtccgggtcagcaccagccggggctgcgtcgccaactgcgccttctgctccgccccgcacctgaagaaccgcgtccaggcgggcaaggcgtggcgcggccgcgggccggaacagatcgtggacgaggtcgccgagatcgtcgaacgccaccagttccggaccttcgacttcgtcgactccaccttcgaggaccccgacggcggccgggtcggcaagaaacgggtcgccgccatcgcgaacggcatcctggagcgcggcctcgacatctactacaacgtctgcatgcgggccgagaactggcacgacacccccgaggaccacgccctgctcgacctgctggtcgcctcgggcctggagaaggtcaacgtcggcatcgaggccggcaccgccgaggaactgctcctctgggagaagcgcgccaccgtcgaggacaacgtcaccatcatcaggatgctgcgggaacacggcatctatctcgccatgggattcattcccttccacccctacgcgaccctggagaccatcgtcaccaacgcggccttcctgcgcgacaattccggccacaacctccggcgcatgaccgaacgcctggagatctaccccggaacggccatcgtcagccgcatgcgggccgacggactcctcggcgagagctatctcgaagggctcgacccctacggctacgcattcaaggatccccgcgtcggacggctcgccaagcatttcgcccagctctacaacaacgacgactaccaccggcacggcgtcatcaccgagcagtcctccgtcttcgccttcgagacctacaacgtcgtactccagaccttcatctcccggctgcaccgccggttcaccaccctgccgggggtggacgaggtgatggaggcattcaaggcccgggtgcacgagatccgccaggagatgggccggcacaactacggcttcttcatgtccaatgtcgaggcggtcatgaacgacaccctcgacccggagaagcagcgccggcaggtggtggacgtcgagcacttcttccgcgaccgcctcgatgtgttgcgcagcgagcaattgcgcgtcggcaaggccctcacccggctcggcgcccgggtgacggaggtcagctcgaccattcccaaggagcgccccggcggactgccgcgccagtacacgggagagggcagcggtgccacgtggtga

SEQ ID NO: 53 - DNA, 1080 bp, Streptomyces lavendulae
gtgccacgtggtgagacgggaaccgccgcggcgcgggtggcggtctgcacgctgagcagcagggaactggtcggcccgctggcccggttgcccggtgtggcggccgcgggcacgctgatgaccgccaacctgggcatcgagcaggtgatcaaggccctgcggtgcgaccggacggtccgcggcctgctcgtgtgcggccgcgactcaccccgcttccgcgccggccagagcctgatcgccctcttccgccacggcctgcgccccgaggacgggcacatccggggagccaccggctatctccccgtcctgaggtcggtgacggcgcgggagaccgaggaggtacgcgcccgcgtcgagctggtggacgcccgtggcgagcgcgacgtcgagacgctgcgcgccgaggtcgcggcactcctcgcccgcgtacggcgcaccccggccctcccctcccgcgagcacgacggcggccaacccagcttcgtggagccggacttcggacggctgcatcctgtcggccgccgccgctccctggacgcgggcatcggcgggttcgtgctcatcagcgtcgaccgtgagcaccggcggatcctgctgcgccactacacctccgatgtgcggccccggcacgagatgtggggcacccgcggggaggcgatgctgctcgggctgctggaggccggcgtcatcgaggaccccgcccacgccggatacctcggcgccgaactggccaaggccgagacggcgctgcggctcggcctgcactacgaacaggacctgcccctgcgcccgccgggcaggccgcccggccctgtgcggcgccggaccgcgaaggagcgaacgaccatggcgcaagcacccgcgctggaggacttcctgcgtctcgtgacgaggacgctgggggccgaggacgccgtcctggacctgcacacgccgctcggcgagcaactggcggtggactccgcccggctcatcgaactcaccgtcgtcctggaggaggagctcggcgcggacctccccgacgacgccgacctcgccagggccacccccgcggaactccacaaagcactcgtgggctga

SEQ ID NO: 54 - DNA, 438 bp, Streptomyces lavendulae
atgcgcagcgtgctgttgctcaacggacccaacctggggacgctcggcaagcggcaaccggagatctacggaaccgacaccctggccgagatcgaggccgccgtggccgaggaggtgggagcgcgcggctgggaggtggtctccgaacagcgcaacggcgagggggaactggtcgatgtgctccagcgccacgacgacgtggtgggcgccgtggtcaaccccggcgccctgatgatcgccggctggtcactgcgcgacgcgctcgccgacttcgccccgccctgggtggaggtgcacctgagcaacgtgtggggacgcgaggcattccggcacacctccgtcacggccccgctggcctccggcgtcgtgatggggatgggggcgctgggctaccggctggcagcgcgcgccctcacccggctggtccccgaggactga

SEQ ID NO: 55 - DNA, 534 bp, Streptomyces lavendulae
gtgggacggtacggaagagagggtctggggatgtcgcgtacggctgaggggaacgccggaggcgtggtggtgccggtggtccggctggtcgccgtgacggacgggccggacgcggagggctggcggcaggcgctcgcccccgaactggtggtggagcacggcgtcgaggcgatcgcggaggcggccggggacggcgggccgtgggcgctggtctgtgccggtgccgggctgggcgcggcgctgcgggccgccgagcgggccgcgcgcccgccggtgcatgtgctgctgtggctcggcagccgcgggcccggcgaaggggtgggcggggaggtctccggtcaatttccctgtccggtcacggccttggtgtccgcggaggtggaccgcggtcgcgccgtggtccccgcctggcgcggcctgaccgaggggccgttcaccgtgcggatcctcccggcggcctgcccgctgcccggggcgtgcgaccaggccggcgctcaggtgatcaaggaggagctgcgggtgtggcccgcctga

SEQ ID NO: 56 - DNA, 765 bp, Streptomyces lavendulae
atggatgcgactttgacgaatgacgtcgagaaagcctcccgggatctggtcgaagccggatactgcctgatcgagtgccccttgccggccgcggtcttcgaaaagctcagagggcggctgctggaggtcgccgagcaggagcgtgagaacggctcggcctttctctacgacggcggcaaccaacgcgtcttcagcctgctgaacaagggcgaggaattcgagcagaacgtgcaggatcccaccgtcatgctcctgatggaggagatcctgggcttcggcttcctgctctccagcacgcacgccaatatcgcgggccccggcggttcccggatgcatctgcacgcggaccagaccttcgcccgcccgccgtggcccccgtatccgctggtggccaacagcatgtggatgctggacgacttcaccgaggacaacggcgcgacccgcctggtgcccggctcccatctgctgggccggcagccggactacgaccggggcgaggggaacaccgagacggtcgccgtgtgcgcgccggccgggagcgtgatggtcttcgacgggcgcctgtggcaccagacgggcgccaacaccaccgaccggccgcggcacggcatcctcaactactactgccgcggctacgtccggcagcagcagaacttcttctcgggtctgcgggaggacgtcgccacccgcgcgacgcccgaactgcgccggctgctggggtacgagaactacttctccctcgggatgaccgacggcctgccgtag

SEQ ID NO: 57 - DNA, 795 bp, Streptomyces lavendulae
atggcacactcaccgcggcggccggacggccccctccgcatcggggtctggctggccccccagcacacctcggtggccgaactgcgcgccgcctggcgcgcggccgactccctgggcgtggactcgctgtggctgtgggaccacttcttcccgctcaccggggaccccgacggcagccacttcgaggcctggaccctgctggcggccatggccgccgacacccgcgccgcccgcctgggcaccctggtgtccaactacgcctaccgcaaccccgacctcctggccgacatggcccgcacggtcgaccacatcggcgacggccgcctgatcctcggcatgggcgccggctgggtcgaacgcgacctgaaggagtacggctaccccacgcccggcgcgggggagcgggtggacgggctcatcgaggcggtggagcgcgtcgaccgcagactcggccggctgcgccccgggccgctcggcgacctccccctgctcatcggcggggacgggcagcggcgcctgctgcgcttcgccgccgaacgggccgccatctggaacaccatggcctggcgcttcgccgagggcaatcgcgtgctggacgagtggtgcgcgcgggtcggccgcgacccggcggagatcgagcgcagcgccttcgtcacccgcgaccagaccgacgaggagctgcgctgcctggtggcgacgggcgtccagcacctgatcttccaggtcgggcaccccttccgcttcgacggcgtggagcgggccctgcgcttcgcgggcggctggagcaaggggtaa

SEQ ID NO: 58 - DNA, 825 bp, Streptomyces lavendulae
atgaagatcagcattgctctgccgaacaccgtgcccggcgcggacgggcgcctgataaccgattgggcgcggcgggccgaggagcggggattcgcctcgctcgcggccaccgagcgcctggtgtatccgggccacgatccgctgctggcgctggcggcggcggccggggcgacctcccggatcgggctgctcaccaatgtcctgatcggcccgctgcgcaccgcgcctgtgctggcgaaggcggtcgcgagtctggactcgctgtcgggcgggcggttcaccctgggggtcgggcccggcgtgcgcgaggacgacttcgaggccgccggccgcgccttcgacgaccggcgcgcggcgttcgaggagcagctggagctgctcggccggggcgcccggccgggcgcggagggccccggtgtgccggtcctcgtcggcggggtcagcgcggcggccgtgcgccgcgtggcgcgctgggccgacggctggacggcgcccggcctggagccggagcggatcgtgccggtcgcggaacgggtgcgccgcgcctggagcgaggcgggacgcgccggggcgccgcatgtggtggcgctggcgcgctacaccctgggcgaggacgtggcccaggagtcggcggccttcgtccgggactacttcgcggtgctgggcgaggaggcggaggagttcgtggcgaagaccccgcgcaccgcggggcagctccgcgcggcggtctcggcgctcgccgacggcggggtggacgaggtcgtcctccaccccacggcggcggcgctgtcccaagtggaccggctggcggacgcgttgctctag

SEQ ID NO: 59 - DNA, 1383 bp, Streptomyces lavendulae
atgcccgctgccggaaaagtcgccgtgataggactcgactccgcgactccgcagtacatgttcgaccggttcgccgaggacatgccggtgttcaccgccctcaggcgcaagtccctgtggggtccgatgcgcagcatcgacccgcccatcaccatgcccgcctggtcctgcatgatgtccggccgctcgcccggcgaactcggcgtctacggattccgcgaccgcggcgcctacgactacgggccgttgaagttcgccacctcccacagcatccaagccccccggatctgggacgagatgacggccgccgggcgctccagcgtggtcctgggcgtccccggcacctatcctcccgcccccatccgcggggccatggtctcctgcttcctggctccctccacacagtcgcgctacacctccccgcccggcctcgccgacgagctggagaagctcaccggcggctacgccctggacgtggaggacttccgctccaccgacctggaacgcgtatcccagcgcgtcttcgacatgagcgagcagcgcttcgaggtcgcgcgccacctggcgaccacccaggagtgggacttcctctccttcgtggacatgggccccgaccgcctccaccacggcttctggaaatactgcgaccccgaccacccgcgccacgagccgggcaacgcctacgccggtctcttccgcgactactaccgcgccctcgaccggcacctcggccgcttcctggagagcctgcccgagaacacgaccgtcctggtcgtctccgaccacggcgcccagccgatggtgggcgggctcttcgtcaacgagtggctgcgcaaggagggttacctcgtcctgaccgaggagcccgccggacccacccccgtcgcccaggccgccgtcgactggaagcggaccaccgcctgggccgaaggcggctactacggacggatcttcctcaacgtcgagggccgggagccgcagggcaccatcccggccgcggagtacgagagcacccgcgacctcatcgcctccgccctggaagcgctgcccgacgaccaggggcagccgatgggcacccgcgccctgcgccccggcgagctctacggagaggtcaacggcatcgcccccgacctcctggtctacgtcggcaacctgcgctggcgggccctggccaccctcggcatgggcaagggcctctacacgacggagaacgacaccggccctgaccacgccaaccacggggacaccggcatcttcgccctcagcgcccccggcatcacccccggccgcgcggacggcctgtcgctgtacgacgtggcccccaccctgcgggaactgctgggtctcgcgccgcagggctcccgcggctccctcctcggctga

SEQ ID NO: 60 - DNA, 1536 bp, Streptomyces lavendulae
gtgaaggcgatggaccgggtggacagagcggtcgagcggttcccgatgtacatcgacgggcaggccgtgcaggcccacgacggcgccgtcctgcgcaccttcgagccggccacgcggcgccacctggccgaccttcccagcggcggcgcggaggacgtccgccgggcggtgtccgccgcccggcgggccttcgacgagggcccgtggccgcggatggcgccgggcgagcgggcgggcctgctgcgcaaggccgcacagcgcttgcgtgaagaagcggagccgctggccgagttggaggcccgcgacaacggctcgacgctgcgcaaggctctcggggccgatgtgccgggggccgcggcagccttcgagtggagcgcgtggtgggcggagcacgtgcccgaacggcagccggaggcgcccggttcgggttcctacgtcgtgtggcggccggtgggggtcgtcgccgcgatcgtgccgtggaatctgccgctgctgctggcggcctggcgcatcgcgcccgccatcgccgcgggcaacacctgtgtgatcaaaccggcttcgttcgcctcgctctccacgctgcgactggtggagctgctccacgagtgcggcctgccgccgggcgtggtcaacgtggtcacggggccgggcggggtcgccggggagcagctggtgcgctcgcccggcgtcgacctggtggcgttcaccggctcggacgagaccggggccgccgtacgggagggtgccgccgcggcggggacgagcgcccggctggacctggggggcaagtcccccaacatcgtgctcgcggacgccgatctggaccgggcggtcaccggcgtcacgtggggagcgttcctgcacaacgggcaggtgtgcatggccggtacccgcgcggtggtgcacgccgacgtccacgacgacttcctgcggctgctgagcgaacgggtgggccggctgcgcgtcggtgatccgctggacccggccaccgacctggggccgctggtctcgcgcaaccaggcgcgtacggccaggcgcttcaccgaactcgggctctcccagggcgcggagctcgtgtgcggcggccgggcgcccgcggcggacgagctgccgcccgggctggacgccggggcgtatttcctgcccacggtgctggcgtcggtcggcgcggacgacgccgtcgcgcaggaggagatcttcggcccggtgctcgcggtcgtccgggccgggtccgacgacgacgcggtgcgcatcgccaacggctcccgctaccggctcagcgccggggtgtggtccgccgatcccgcgcgggcccgcgcggtggccgagcggctgcgcgcggaccgggtatggatcaacgactaccggctggtcgacctggagctgcccggcacagccgggccccgctccgccgtctgggaccggctcaccaacgagctggacgcctaccgccacaagcacgtggtgcacggtggcggtgcgggagcgggcggggtgccggcgccgcccactccctacgcgctgctgggcgggtga

SEQ ID NO: 61 - DNA, 1419 bp, Streptomyces lavendulae
gtgaaaccagccagccactccgtgacggacacgtccgcggccctcggcgccgcggccgccgaagagctcgcggcgcaggtcgccggatccgtcctcctgcccggggacgaggggtacgacgaggagcgctccggcttcgaactgtccgtggaacaccgccccgccctcgtcgtcgtcgccaccggtgccgcggatgtcatcgccgccgtgcgcttcgccagggcccggggccttgggatcgccgtccaggccaccggtcacgggaagtcctcggcggccaccgacgtcctcatcagcacccggcggatgaccggcgtcagggtcgacccgcgggcccggaccgcccggatcgaggcgggcgtgcgctgggagcaggtgatccacgaggcggcggcgcacggtcttgcaccgctgagcggctcggcgccgttcgtcggcgcggtctcctacctcctcggcggcgggctcgggcttctgtcgcggaagtacgggttcgccggcgaccatgtcgtctcgctcgacctggtgacggccgacgggcggtttctccaggtctccgccgaggaacaccccgatctcttctggggcgtgcgcggcagcagggggaacctcggcatcgtcacctccgtcgaggtcgggctgttccccgtcacccaggtgtacggcggagggctgttcttcgacgccggctccacgcgcgccgtgctgaacacctatctccagtgggcgccccggatgcccgaggacatggcgtcgtcggtgttcctggccgcgtatcccgatgccgagggggtgcccggaccgctgcgcggccggttcgtcacccacatccggctggcctggctgggagaccccgaggagggtgagcgccggttcgccgagctccgggccgccggcacggtcgtcatggatacggtggacacgctcccgtacacgcgggccgggatcatccacaacgatccgccggccccggtgtcgagtcacagcaaaacggtcatgttcgggcagctggacgagatcgccgtcgacgagatcctcaggctcgcggggccgggcacggacgcgctgttcggggtggagctgcggcacctgggcggcgccctcgcccggccgccccggcacccgagcgcggtgggccacttcccggaggcggtgttcaacgcctacgtgggctcgctggtcgacccggacaccctggcggccgtggacgcggcgcagcaggagttcgtcgacagcatgcggccgtggacgacgcccggggtgtgcctgaacttcctcgcgggtcacaacacatcgagggagacgacccgcagcgcctacacgccggaggactacgcgcggctccaggccctgaagtcgcagtacgacccgggcaacgtcttccggttcaaccccaacatcccgcccctgccggcctga

SEQ ID NO: 62 - DNA, 1188 bp, Streptomyces lavendulae
atgacctcagccgccccgcccgcctttcccttcccgcccggccccggcggcacggtgccgcccgagtacgcgcggctgctcaccgatgacccggtcgccgaggtgcgcctggcggacggctcgcgcatctggctggtgacccggcacgaggacgtgcgcacggtgctcaccgacggccgcttcagccgccatcgcgccgccatgctgccgggctcgggcttcggccggtcccagggctcgggcatcgtggacctcgacccgccggagcacggccggctgcgcggtccggtggtggccgcgttcggtgcctcgcgcacggcgcggttcgcaccccgcatcgaggcggccgccgaggcggccctggaccggctgcccgccggcagcggcacggtggacctcgtcgcggcgtacaccgcgcccttcgccggccgcgtcacagccgagttcctcgggctgcccggggaccggtggcaggacgtcacctccgacgtcgagctgctgctgcttccgcgcggtgccaccgagcaggcgctgaaggaggcccgcggcaggctcggccaggtgctggacgaactgctcgcggcccgcagggccgagccgggcgacagcgtcaccgacacgctgctggacgcggaggagctcaccgacgacgaccggcgcctgctgctccacggcctgatcatctcgggcttcatcaccatccgcgacctgctggcccggcacctcttcggcgtgctctcctcccccggcctcgcggcccggctgcgcgaggacccctccgtactgccctctgccgtacaggagttgctgcgctactacccctccagcaacgacggcctgctgcgggtcgccaccgaggacgtggtgctctccggcaggcgcgtcgccgccggggacgccgtgctgccactggtctcggcggcctcccgcgaccccgaggtcttcgccgatccccacgtgctcgacatcgagcgggtggccgaccgcggcatcgcgttcggcgccgggcagcacgcctgccccgcgaccgggctggccgtgaccgaactgaccgtcggcatcggccgcctgctggcggccttcccccgcatcgccctggccgtgcctcccgaagaggtcgagcacagctccgaactcctgcccctgggcgtccggtcactgccggtggtccccggcccgcgcaactga

SEQ ID NO: 63 - DNA, 1425 bp, Streptomyces lavendulae
atgcttcctgagttccaattgcagtggaattggctcgacgccccggccggcggcggaggcgagctgcaagcgacctgggcccggctgcgcatcgccgtgggcgccgagaccgtcacactcgtccaggagcccgggcaggggaccttccgggagcacacgaccggctcgctctaccccctggccgagtggatcgccttcaactggtggtcgctggtggccgacgcgcggcccggcacccagatatcccagctgcgcttcgcctaccgccacggtgtgggcgacaaccgcggttcgtggtggatgcgttcgcgccgtcacatcctgcgcgccgcctgcgacggcttccgctggccggacatgctcttcgtgcccgagggccgggagacccggatcgtatggatgccggacatgggccccgacgtacgacccgggaaccgcttcgcgagccggggcaactcctgtgtggagagcgccgcgttcaccgccacactggcctcgttcgtcgacgcggtgaccgagcgcctcacggaccagggcatcaccggcaccccgctccaggaggagtgggccgccgtccgcgccaccgacgaggacgaggccgccttctgccgcatcgcggcacggctgggcctggacccctacgccgaggccgagccgtacgaggcggacatcctcaaggccgccgagcagttggcggaaccgctcgccagtgacttcttcaacggggtgcggcctgagcggatagccgaccagctccagtggatcgcgcgcgtccgcaccctgatgggcaccgcgcccgcggataccccgctccctcccgccttggtggaactgcgcaaggactgcgccgacttgagcgagaagttcttcgctccggggcgactcgacaacccctgggacctcggctacgaggtggcgcaccgggtgcgcgcgtgggcgggtctggacgacaccgcgcccttcgacccggcccccctgatgggctaccgcaccgagcaggtcccctatatggaccggggcctggtcgccctcggcacccgcaggggcgcggacgggccggtcctggtctcctcccggcgcttcaccgaccgcccgcgccgcttcctccaggcccgcgcgctgtggcatctgatctgcgaccccgacgacaccttcctgatcgcggcggcgcacacccaccgccagcacgtggcccgcggcttcgccctggaggtcctggcccccgccaagggcgtggcgaccctgctggccgaccccggacacctggtgtccgccgaggacgtcgaggtcatcgccgacgactacggctgcggcaacatcgtcgtggaacaccagctggacaaccgcgtcctggcgaaggacttcacctggccgggccacgcccgcgccggcgcgccggccggtgagaggagccggggcgcatga

SEQ ID NO: 64 - DNA, 1332 bp, Streptomyces lavendulae
gtgacaatccgccagcgtgtcgtcgtcgtcatcaccgagggagcggcacccgagctgctcgaccgctggtgtgcccaggggctgctgcccggcttcgccgccctgcgctcgcagggggcttccgggccgctccacgccgagggcaccccctacgaaccgccgggcctgctgagcgtcctgaccggccggcgcgccgcggaccacggcttctactcctactggacctgtcacgacccggagtacgcgccgcaggtcctcacccccgagcaccgccgccacccactgctgtggcagcacgaggtgttccagggcgtcaggttcgcctcgataggcctcttcggcacccatcccccggagcccttcgacggttccctgatcacctatccgatgtatgccaccctccacgcctgccacccgcgcagcctccagcgcaccctggcgaagaagggcatccgtccggtccacgacgtgtcgatcttctggaccgggcaggaccgcgacgagctgctgccttccctgctggaggcggacgtgcagcgcgggcgcgcggcattggctctgctggaggagtccgatgtcgtgatcgtcaacctcacgagcatcgaccgctgttcgcacatctactggcaggagctggagcacggccccgagcacgagcgggagagcgccgtcttcgccgcctaccgcacctgcgaccaggtcatccaggacgccctgcgggcggccgacgaccgcaccagtgtcgtggccttctcggagataggcttcgggccgctgcgcaactactgttccatcaacgacgagatggagcaggcgggtttcctggccaccgccgaggacggccgcgtcgagtgggccggcagcgcggccttcgaggcggtgcagggcacgcacggggtgaacatcaacctgcgcgaccgctacaagcacggcctggtcccggagcgcgactacgagaaggtccgcaccgacgtcgcggccgcgctgctggagcggcgcaacccccgtaccggcaggctgttcttcgacgcggtgcgccgccgggaggaggtctatcccggcgaggccacccagcacgcccccgacctcatcctggagccggcggactggcgctatcttccgctgggcgacccgcactgggcctcgcacgtccaccgcgactggcagagcggctggcaccgccgggagtcctactggtcggccgtcggccccggcttcaccggtggggcgcggcagacccgcaccgccgcccccgtcgatattcccgcgaccgtatgcgctctgctcgggcgtgacgtgccgaacgactgggacggcgtgccgctgtcctga

SEQ ID NO: 65 - DNA, 372 bp, Streptomyces lavendulae
atgacaccagaggaactctccgacttcgcgctggagctgccggaggcggtggacgacgaggcgttcggccccggagccgcggtcttcaaggtggagaagaaggtcttcgccattctccaggacgcctccgaggaccgcccgccgcaggtcacgctgaagtgcgaaccggatctggcgctgcacctgcgcgagcagtacgcggcggtggtgcccggctaccacgtcaacaagcgccactggaacacggttgtcctgaacggcacggttcccgtggaggagctgcgggagatggtggagcattcgtacgatcgcgtggtggcggggctgcccaaggcggtacgggaacgtctgcgcctcctgcgcaccgtgtga

SEQ ID NO: 66 - DNA, 1056 bp, Streptomyces lavendulae
atgaccgtggagcagacccccgagaatcccgggaccgcggcccgcgccgccgcggaagagaccgtgaacgacatcctgcaaggggcgtggaaggcccgcgccatccacgtggccgtcgaactcggcgtcccggaactgctccaggagggcccccgcaccgcgaccgccctcgccgaggccaccggcgcccacgagcagaccctgcgcagactgctccgactgctcgccacggtgggcgtcttcgacgacctcggccacgacgacctgttcgcccagaacgccctctccgccgtcctgctgcccgaccccgcgagcccggtcgccaccgacgcgcgcttccaggcggccccctggcactggcgggcctgggaacagctcacgcacagcgtccgcaccggtgaggcgtcctttccttcgacgtggccaacggcacctcgttctggcagctcacccacgagggaccccaaggcgcgcgaactgttcaaccgcgccatggggtcggtctccctcaccgaggccggacaggtcgccgcggcctacgacttctccggcgccgcgaccgccgtggacatcggcggcggccgcggcagcctcatggcggccgtcctcgacgccttccccggcctgcgcggaaccctgctggagcgcccgcccgtcgccgaggaggcccgtgagctcctcaccggccgcggcctcgcggaccggtgcgagatcctgcccggcgacttcttcgagaccatccccgacggcgccgacgtctacctcatcaagcacgtgctgcacgactgggacgacgacgacgtcgtacgcatcctccgccggatcgccaccgccatgaagccggactcccggctcctggtcatcgacaacctcatcgacgagcggcccgccgcatcgacgctcttcgtcgacctgctgctgctcgtcctcgtcggcggcgccgaacgctcggagagcgaattcgccgcgctgctggagaagtcgggcctgagggtggagcgctcgctgccctgcggcgccggcccggtgcgcatcgtcgagatccgcagggcctga

SEQ ID NO: 67 - DNA, 1641 bp, Streptomyces lavendulae
atgacggtgctgggtctgggtggatccggacatgactgggcctcctgtgccaccgacggccgacggctggtggcgatcgacgaggagcggctggtccgcagcaagtacggcctgggagcggacctcctggcgggccacagccggcgcgccgtcctcgacgccctcggcacgagtgccgaggccgtggaacacgtggtggcctgcgagctcgtaccacgccccttctaccactcgttccgcaggcgcgtgacggtcgtcaaccaccatctcgcccacgcctacagcgcgttcggggcctccgggatgacccgcgccgccgtactggtctgcgacaactccggcagcctggtgacgggcctgaagtccggcccagggccgcgcgaggcggagacgatcagctgctacaccgccgacgcctccgggctgcgcctggtcaaccgggtcgccgggacacacgccgtggacgcctcctccgagagcgcctactaccagcccggcgagaccgacaattccctcggccacttctaccgctcggccagcctcgcactcggcctcgcctactccggtcccaagacccgctaccccgtcagcgaggacggcaagaccatgggcctcgcgccctacggcgacgaccgcttcgtcgacgaggtcgcggagctggtcaccctgctgcccgagggcggcgtgcagatctcggcgagcaaggtgaaccacctcttcgaacgcctcgtggaatcgggtgagttcgaggaccgggcggccttggcctacgccgcccaggagacgctggaacgcgccctgctgcactgcgcccgcgacctgcaccgccgcaccggcctgacggacctgtgcatcgccggcggcgtcggcctcaacagcgtcgccaacggccggatcctgcgcgagacccccttcgagcgggtcttcgtcgtcccggccgcgggcgacaacgggatcagcctcggctgcgcctactacggcctccacgagctggaggggcgcgcgccgtcggagctccccgccctcgacaccgcctacctcgggcccgactaccccgccgagcgcgtcgacgcggcgctggccggctcgggcttcaccgtggagacccccgacgacctgcccggcagggtcgccggcctgctcgccgaagggaagatcatcggctggttcgacggccgctccgaattcggcccgcgcgcactgggacaccgcagcatcctcgccgcacccttccccgcctccgtgcgggaccacctcaacgacaacgtcaaacaccgcgagtggttccgcccctacgcccccatcgtccgcgaggaccgggcggcggactacttcgacctcgtccagccctccccgttcatgctggtcgtcgcgcgcgtgacccggcaggacgccatccccgccgccacccacgtggacggcaccgcccggctccagacgctgaacgccgcacagaacccgaaggtctacgagctgctcggcaggttcgaggcgctcaccggctgcgccgtgctgctcaacacctccttcaacgtcgccggccagcccatcgtcgagaccccggaggacgccgtcgaggcgttcgcgggcatgcgcctggaccacctcgtcgtgggggaccggctggcgaccaagccctga

SEQ ID NO: 68 - DNA, 1707 bp, Streptomyces lavendulae
gtggacgtccccgtgctcgtggtcggaggaggaccgacgggcttggcgatggcgctcttcctcgcacgccacggcgtcggctgcctgctggtcgaacggcggacgaccacctcgcccgtcccgcgcgccacccacgtcagccgccgctccatggaactcttccgcgaggcgggcctggaggaggagatccgccgggccgggttcgaggtcgtgcgcgaggacgacccacggctgcggacccggcccgaacgccacctgccccgggtggtcctgcaagccgcctcgctcgccggccccggcccggtgggggtcctggagaccggtgacgaggaactggccgtacccggcccctgcgcacccttctggtgcggccaggaccggatggaacccctgctcgccaaggccgcggcgcgccacggcgccgatgtgcgcttcggccacgaactgaccggcctgtggccgggggaggacagcacacgggcccgcgtccgggcagcgggaacgggacggacctacaccgtcgacgcccgcttcgtcatcgccgccgacggggcgcgcggcgagatcgccgagcgcgtgggcatcgcgcgggagggcctgggcacggtcgcccaccgggtgagcatcctcttccgcgccgacccggggcgctgggcccgcgaccggcggttcttcatgtgcatgatccagaacccggggttcgacggggcggtgatggagctcaacaccccgggccgctggtgcgccgcggtggactacgacccggcccgcgccgaacccgacggcacctactccgcacgcacctgcctcgacctggtccgggccgccgtcggtgacgaccggagcgacgcggcggtcgacaccgtcttccactggaaggcccggcaccgcatagcggccgcctaccgcagtggggcggtgttcctcatcggcgacgccgcccacctccacccgccctccggcggctacggatccaacgtcggcttccaggacgcgcacaacctcgcctggaagatcgccgccgtgctcggcggctgggccggaccgcggctgctggacacctacgacgaagagcgccgccccgtgggaaaggcgacggcggagcagtcgatgctcctcgacggcgtgccaccggaaccactggggggaagcgtcgtccgctgcgatccccgcaccctgatcatgggataccgctaccactccgccgccgtcctcggccccccgcacggccccgccttccccgcggccttcaccctgcgcggagacccgggcacccggctgccgcacgtatggctgcgtacggacgcgggggaacgcgtctccacgctcgacctgtgccacgggcacttcgtcctgctctccgccgacccggtctgggcggcggccgcggcgcgctcggcgaaggagacgggcgtaccgctgcggggccaccacctggcggccaccggaagcgaactcgccgacccctccggcgagttcccgcggagctgcgggaccgggcccgcgggggccgtgctcgtacggccggacggcatggtcgcctggcgcacggcccgcgccgtgcccccggacccggacagcgcgcaggacctggtcacggcagcggtgagacgtgtcctcgcactgccggagcgcgcggcgccaccggtgctcggtccgccgcggttgtcacgcggttcctatcggcgagtcgggagcgacgggtga

SEQ ID NO: 69 - DNA, 483 bp, Streptomyces lavendulae
gtgaagcctcattccttctgcacgtgctggccgggcgccaccgtatggctgacgggcccaccgggcgcgggcaagacgacgatcgcccgcgcactggcggagcggctgcgcgaacggggccggcgcgtggaggtgctcgacggcgacgcgacccgcgcgctcctgaccgcgggctcctcgtgggaggaccgtggcaccggcctccagcgggtcggcctgatggccgaggtcctggcgcgcaacggcatcgtcgtcctcgtcccggtgaccgcggcccgcgcggacagccgcgaagccgtacgcagacgccacgagcggtccggcaccgcgcacctggaagtgcgggtggtccgggacgcagtgcctccgagcgggctccccgcgccgcccggcccagatctgcggatcgcggcgcacgagcagagcgccgaggagtcggcgcgggcactgcaccggctcctggcggagagggagctggcgtga

SEQ ID NO: 70 - DNA, 960 bp, Streptomyces lavendulae
gtgaaccccgggcgcggtggagcgtacgccgcggggcgcgacgggacccgcgggacgcgacgccctcacggtctgtcgcacctggatctgctggagtcggagtcggtccacatcttccgtgaggtggcgggcgagttcgagcggccggtgatcctcttctccggcggcaaggactcgatcgtcatgctgcacctggcgctgaagtccttcgctcccgcacccgtgccgttcgcgctgctgcacgtggacaccggccacaacttccccgaggtgatcgcctaccgggaccgcgtcgtggcggcgctcggtctgcggctggaagtggcctccgtgcaggacttcatcgacaacggcaccttgcgcgaacgcccggacggcacccgcaatccgctgcagacggtgccactgctggacgcgatcgggcgccaccgcttcgacgccgtcttcggcggcggccgccgcgacgaggagaaggcccgcgcgaaggagcgggtgttctccctgcgcgacgagttcggcggctgggacccgcgccgccagcgccccgaactgtggcggctctacaacggccgccacgcacccggcgagcacgtccgcgtcttccccctctccaactggaccgagctcgacgtgtggcagtacgtcgcccgcgaggagatcgaactccccaccatctactacgcccacgagcgcgaggtcttccgccgcggcggcatgtggctggcaccgggggagtggggcggcccacgcgagggggaagcggtggagaagcgacgggtgcgctaccgcacggtgggggacatgtcctgcaccggcgcggtggactcggcggcggccaccgtggccgacgtcgtcgccgagatcgccacgtcccgcctcacggaacggggcgcgacccgggccgacgacaagctgtcggaagccgcgatggaggaccgcaagcgcgaggggtatttctag

SEQ ID NO: 71 - DNA, 492 bp, Streptomyces lavendulae
gtggggcaggacagccggccgcggtggctcaccgacgaggaacaacgcgtgtggcgcggctatctgcgggccaccaggctggtggaggaccacctggaccgccgcctccagcgggaagcggacatgccgcacctctattacggtcttctcgtccagctctccgaggccccgcgccgggggatccggatgaccgaccttgcccgcaacgcgaagatcacccgcccgcggctctcgcacgcgatcacccgcctggagaagctcggctgggtgcgccgggaatcgtgccacggcgacaggcgcggccagaacgccgtcctcacggaagagggccgcgaggttctggagaagtcggcgccgggccatgtcgccgctgtgcgcgcggccgtcttcgacagcctcaccccggaacaggtcgggcaactgggccggatctgccaggcgatagagaaggggctggaccgggaaggcgcggacctgccgtggctgcgctga

SEQ ID NO: 72 - DNA, 1242 bp, Streptomyces lavendulae
gtggaacgacacgacggggcaccgggctggggcttcacccatacccagtacagcgcggaccacggtgaacgcggcgccacccgcagggccggggccctgctctccgcgcggcccctgccgcagaaccagcacatcatgggctggggcgcggagaatcccgaaccggcgcccggacgctacgacttcgaggtcctcgacgagcgcgtcgccctgatgcgcgcgacgggggccacgcccgtcctgaccctgtgtgccgcccccgactggatgaagggcggccggcccggccgcaccgactggtcgcgactggagaccgcccccgacccccggcactacgcggacttcgcccggctcgcgggcgtgatcgcccaacgctacccggacatcaggcacttcctcgtgtggaacgagctgaagggcttctacgacgaggacaggcggcgctgggattatgagggatacacccggctgtacaacctcgtccacgccgagctgaagcggcggaacccgcgcaatctggtgggcggcccctatgcggtggtcgaccacgacccgcccgccgaggacgcggcggaccgctcgcgcgaactgcgcggtccctggggcgagctggaccagcgctccgccgacgtcatccgctattggaacgcccacaaggcgggcgcggacttcgtcgtcgtcgacgggtccagctacacccgcgagggccaccgggcgattccggacgagttcgccgccaccgagaagttcgccgacgtcacccgctgggtcaggagcgtgaccggactcccggtgtggtgggccgagtggtacgtcgagccgcccgccgaggacgaccggccgggcggccgggacggctggggcgaggggcaccgcaccgccgtgcaggccaccgcgatgatgcggctggcggagagcggcgcgtcggccgccttctactggaacccgcagcggaccgggaaggcgtgccccggctgcctgtggcggagcacccacttgcgcgacgggggaggggagttgcccatggcgggtctcctgagccggttcgctcgcgaattccctccgggcaccgccttccggccggtcgccgtcacctgcgggagcggtgacagggtcgaggccctcgccgacgaggccgccgtgctcgtcgtcaacaccgagtgccggccggtggccgccagggtggacgggcaggcgctgtccctcgcgccgtacgaggtgcgctggctgacccgcccgtaa

SEQ ID NO: 73 - DNA, 816 bp, Streptomyces lavendulae
atgacgcgaaggcgcccaacgggcccgattcaccgtcggcgggcgtcactcaccctttcccccacgggagccgccatgagaagaaatcgcatcgccgccctgctgccggccgctctggcactggtcggcatatccgtcctcgcccccgccaccacggcgagcgcggccgcaccgcacggcggcacctcgcaggccgccgcattccccgtgagcgaggcccagttcaagcagatgttcccgaagcggaacgcgttctatacgtacaagggcctggtcgccgcgctcaaggcgtacccgggcttcgcgggcaccggcagcgccgaggtccggaagcaggaggccgccgccttcctcgccaacgtcgcccacgagaccggcggactggtctatgtcgtggagcagaacaccgccaactacccccactactgcgaccggagccggccctacggctgtccggcaggccaggccgcctactacggtcgcggcccgctccagatcagctggaacttcaactacaaggcggcgggtgacgccctcggcatcgacctgctccacaacccctcgctggtgcagaaggacgcggccgtctcctggaagaccggcctgtggtactggaacacccagcgcggccccggcaccatgaccccgcacgaggccatggtcaaccaccgcggcttcgggcagaccatccgcagcatcaacggcgccctggagtgcgacggccacaaccccgcccaggtccagagccgcgtcgcgaactaccagcgattcaccaagatcctcggcgtggcgccgggcggcaatctctcctgctga

SEQ ID NO: 74 - DNA, 12249 bp, Streptomyces lavendulae
ggatcccgatcgtctcggacatgaccggcgaccttctcggcgcgcgggaggcccaggaccccgcctactgggtgtcccacatccgccgcgcggtgcgcttccacgaccagatccgccgtctgcagcgctacggggccggggccttcgtcgaggtcggcccggacacggtgctcagctcggccggccaggcgtgcctgacggaccaggcgggcaggagcgcgcccgtcctggtgtccctcgcgcacgccgagcgcgcggaggtgcccgcgctcctgaccgctctggccaccctgcacacccgtggcgtggccgtggactggcgggcgtggttcggcgacgggccgcgcgcggccggcctgcccacatacgcgttccagaagcagcactactggccgtcgggccccaccggttggcggtccgggcccgcccccgtacccctgccccaggccggaacggaggacgccgaaaggcccggtcgcgccgcggagtggcgggcgctgccgcccggtgagcggtacgacgcgctgctgcggatggtgcgcggcgaagccgccgccgtgatggggcacgccgggccggaggcggtggagccggagcgcggcttcctcgaccacggcttcgactcggtgatggccgtgaagctgcgcgaccgtctcgtggccgggacggggcgggagctgccgacgaccctgctgttcgaccaccccacgcccgcggccgtcgccgactacctgctggcggggacgggcgaggccgagacggcgccgtccgtgtccctgtcggaccagctcgaccgcctggaggccgacctcgcgcggctgccggccgacgaccggcagcgcgcccgcgtcgccgagcggctcaagggcctgctcgcggtccacgcgccggaccggggcgccgggagcgaggacgcgccggaccaggacgcgctggacacggcgaccgacgacgagatgttcgagctgatcgagaaggaactccgccgtggatgagaccaacgagaccaaactccgcgagtacctgcggctggtcacggccgatctgcggcgaacccgcaggcagttggaggaggccgaggacgcggcccgcgagcccgtcgcgatcgtgggcatggcgtgccgcttccccggggacgtggcatcgccggacgacctgtggcagctggtcgccgagggccgggacgccgtcaccgagttccccgccgaccggggctgggacgtcgacgccgtctacgaccccgagccgggcaccccgggcaggacgtacgcgcgccacggcggcttcctcaaggacgccgccggattcgacgccgccttcttcggcatcacgccgcgcgaggcgctcgccatggacccgcagcagcgcatgatcatggaggtctcctgggaggcgttcgagcaggcgggcctcgacgcgaccaccctgcggggcgaggacgtcggcgtcttcgtcggctccaacagcaacgactacctgatcaacgtgctcgacgcgcgggacgtcgccgagggcttcatcgggaccggcaactccgccagcatcctctccggccgcgtcgcctacaccttcggcttcgagggcccggccgtgtccgtcgacaccgcctgctcctcctcgctggtcgcgctgcacctggccgcgcagtccctgcggcagggggagtgctccctggcgctggcgggcggcgcgacggtgatggccacgccgaccgccttcatcgagttcagccgccagcggggcctggcccccgacggccgctgcaagtccttctcggcgaccgccgacggcaccacctggtccgagggcgcggccgtgctgctgctggcccggctctcggacgcccgccgcctgggctaccccgtgcacgcggtcatccggggcagcgccgtcaaccaggacggcgcgagcgcgggcctgaccgcgcccaacggaccggcgcaacagcgggtgatccggcaggcactggccaacgcacggctgacggccgacagcgtcgacgcggtcgaggcacacggcaccggcaccccgctgggcgacccgatcgaggcccaggccctcctcgccacctacgggcgggcccgcggcgagggcaggccgctgtggctgggctcgctgaagtcgaacctgggccacacccagtccgcggccggcgcgggcggcgtcatcaagatggtgatggccatgcggcacgggacgctgccccgcacgctgcacctcacggagcccaccccgcgcgtcgactggtccgccggtgacgtacggctgctgaccgaggcccaggactggccggacaccggacagccgcgccgtgcggccgtctcgtccttcggcgtcagcggcaccaacgcccatgtgatcctggagggcccgcccgccgaggaggcaccggacgcgccgctgccggacgtctcctcgcagccgcggggcccgctgccgtgggtcgtctccggccgcagcgaggcggccgtccgagcgcaggccgagcgcctggcggcccacctgaccgcgcgcccgcacctggcaccggccgacgtggccaccgcgctggccaccacgcgggcggccttcgaccaccgggccgccgtcgtcggccgggaccgtgaggaactgctcgccggcctcgcggccctggccaccggaacccgcgcgcccggcctggtcaccggccggaccccgccgtccggcggcaaggccgccttcctcttcaccggacagggcagccagcagcccggcatgggccgcgaactggcggctcacagcaccgtgttcgccgacgccctggacgaggtctgcgcccagctcgaccggcacctcgaccggccgctgcgcgaggtgctgttcgccgcggacggcacgcccgaggccgccctgctcgacacgacggcctacacccagcccgcgctgttcgccgtcgaggtcgcgctgctgcggctgctggaggactggggcttgcggcccggcatggtcgcgggccactcggtcggcgaactgaccgccgcctacgccgccggggtctggtcgctcgccgacgcctgcgccctggtcgccgcccgcggccggctgacccaggcactgcccgcgggcggcgccatggtcgccgtgcaggcgaccgaggacgaggtgcgcgcccaactcgccgacggccgccccggcgtggacatcgccgccgtcaacggaccggaagcggtggtgctgtccggcgacgaggccgccgtcacggacctggcgcgcgagtgggccgcccgcggccgggagaccaggaggctgcgggtcagccacgccttccactccgcccacctggacgccatgaccgaggcgttcgccgaggtcgcacgaggggtgtcctacagcgcgccgtccctcccggtggtctccacgctcaccggggcccccgtcaccgacgagctccgcaggccggaacactgggtgcggcacgtccgggagacggtgcgcttccacgacgcggtccgcgccctgcgcgaccgcggggccaccgcgttcctggaggtcgggcccggcggcgtgctgacggccgcggcacgccgatgcctgcccgacgccgcccccgagacgttcgtccccgtgctgcggcgccgcaggcccgaacccgagtccgtgctgacggccgtcgcgcaggcccacacgatcggcctctcgccggcgtgggaccgcctgctgcccaaggcccggacgcgcgtggacctgcccacgtacgccttccagcgcggccactactggctggcgggcatggccggagcgggcaccgcgcggccggtgcggccggaagtgcaggagcccaccgccccctccggtacgccgccgctgtcgcgacggctggccgacgcgtcggaggaggagcgcggccacctgctgctgacgctggtacgcgagcagtcggccaccgtgatgggcggcgtcgaccccgcgcaggtcgaacccgaccgccccttcctggagctcggcttcgactccctgatgggcgtcgagctgcgcaccgcgctcgccgccgactgcgcactgcccctgccgcccggcctgatcttcgaccaccccacgcccgccgccctggccgccttcctcggcgagcagctcgcggcggcggcctccggcacccccacggcggcggcaccctcgccgtactccctggaggcgctgtaccgcaacgccaacaccctcgaccggcccgaggacgcgctcgccctcaccaaggccgcctcccggctgcgcccggtcttcgccagcgtggccgaggcggggcaggacccggtcacggtggagctggcacaggccaccggccttccgggcctgatctgctgcccggcacccgtgccgctgtacggggcacagcagtacagccggctcgcagccgccttccgcggcacgcgcggagtctcggccctgctcgcccccggcttctccccgggcgaactgctgcccgccgacttcgaggtgatgcaggacttcctcgccgagggggtccggcggcagaccgacggcgcgcccttcgtcctcctgggccactcctccgggggctggttcgcctacagcctggcggcccacctggcgcgcaccgggccgcgcccggaggccgtcgtgctgctggacacctatcagctgcacgacccggcgctgcaccgcatgcagcgcgaactcgcccagggcgtcctggaccgcgaggaggacttcggggcgatgacggacgtacggctgagtgccatgggcaaatacttcgacttcttcaccgactgggtggccgaggacgccggtgtcccgacgctgctgctgcgggcctccgagcctctgggcgaggtcgtcgagggccaggagtggcgctccacctggccgttcgacagcacggtcctcgacacggaaggcgaccacttcgccatggtcaacgaccacgcgccgcggacggcccaggccgtgaacggctggctgtcgggcctcaccggcggaaggggctgagcgccggtggagacacgcaacgccgaacggccgtggatacgcagcttccaccccgctccccaggcccctgtgcggctgctgtgcctgccgcacgccgggggctccgcgagcgcctacttcgcgctgtcgagggaactggcgccccgggtggaggtgctcgccgtgcagtaccccgggcggcaggaccggcgcgacgagccgctgctggactcgatcgaggccctgcgcgacggggtcgccgaggccctgacgccctggctggaccggccggtcgccctcttcggccacagcatgggcgccgtggtggcctacgagctggcgcggctgctgtgccaggacgcgggcgtgccgctcacccacctcttcgtctccggacgccggggatccgaccgaagtctccgtccttgccgccgtgttccggaattcaccgtgacaccgccgcgcggctcttcttccgaagtcctccagatccggcacgagtttgtatccgaacggggttctgcgtgcgaaatactctcttcgaattgggtgacatacccccgatcggcaccgtacccgagcagatgtacgcctcggtgatccgacgggagcgctacggacagccccaccaggcgttccgcagcgaggtcgtggacgtgccgaaggtggggcccggtcaggcgctggtcctcgtgatggccgcgggcatcaactacaacaacgtctgggcctccctggggcagccggtcgacgtgatctccgcgcggcagaagcagggccacagcgaggacttccacatcggcgggtccgagggctccggcgtggtgtgggcggtgggggagggcgtcacccaggtcgcggtgggcgacgaagtgatcctctccggctgccagtggacggagacggccgccgacatccggctcggcgccgaccccatgacctccggctcgcagtcggtgtggggatacgagggcaactacggctccttcgcccagttcgccctcgtcgacgactatcagtgccaccccaagccgcccggcctgacctgggaggaagccgcctgcttcctgctcaccggggccaccgcctaccgccagctgtgcggctggcagccgcacgacgtgcggccgggcgacccggtcctcatctggggcggggccggcgggctcggctccatggccatccagatcacccgggcgcggggcggcatccccgtcgccgtggtctccgacgaggagcgggcccgctactgccgggagctcggcgcccagggcaccatcaaccgcctggacttcgaccactggggacggctgcccgacatcggcgaccacgaggcgatgggccgctggaccgagggtgtacgggccttcggccggcgcttctgggaggtgctgggcgagcgcaggtccccgcgcatcgtcctggagcacagcggccaggccaccatccccacctcgatgtacctgtgcgacaacgcgggcatggtcgtcatctgcggcggcaccaccggctacaacgccgacatcgacctgcgcttcctgtggatgcgtcagaagcgcttgcagggctcgcacttcgccaacctgcggcagtgccgcgacgtcatccacatggtcgcgaacggccagctcgacccgtgcctgtcgtggaccggcggcttcgacgacatcggcaaggcacaccagctgatgcacgacaaccagcacccccagggcaaccaggccgtcctggtcaacgcgccgcggaccggcctgaccaccttcgcctgaaccaccgccccggtgttccgacgtcttccccccacacttaccgaccaaggagagatcaccatggacaagctcgacatcctctggagcgagcgcgagatccgtgccgtgctgcagcgctactgccgcgggctcgaccgcctcgacgaggaactggtcaagtccgcctaccacgaggacgcgcacgacgaccgcggcgtcatccgcggcaacgcacacgacttcgtcaagcagatcgtcccgctcctgcgcgacgcctacaccggcaccctgcacaccctgcacggcagcacgatcgagatcgacggggatgccgcgggcgtggagtcctactgcaccgcctaccactaccgcgagagcgacggcatcaagcgggtggagcagttcgccgggcgctacgtcgaccgcttcgagcggcgcgacggcgtctggaagatcgcccgccggctcgtgctgaacgacttcagcctcgcccaggaggtgccgctcgaccccgccgaggcccaggccggcttcaacccctcccaccgcgacctcaccgacgccagctaccaggtgctgccgctgcgcggcccggacgcccccaccctctgagccgtccggccgccccaactcgccccacctcaccaggagtcaccaccgtgtccgacaccgagcagcacgcgcccacgctgccgcggcagcgcacctgccccttctcgccgccgcccgagctcgaggagctgcggcgcaccgatcccatcagcaggatgcggttcgccgacgactccccgggatggctgctgacccgccacgccgacgtccgcgccgcgctggccgaccccggcgtcagctcgcaccccggcaaggcaccccagccctggcgcaacctcgcccccgagatgcgcgccgagcactacctgccgggcttcctgatcttcatggacccgccggaccacacccgctaccgccgcctgctcaccaagtggttcaccatgcgggccatccgcaagctcgaacccaggatcgagcagatcgtcaccgagaccctcgacgccatggaggcccagggcggcaccgtcgacctggtgcagtccttcgcgctgccgatcccgctgctggtcatctgcgagctgatgggcatccgctacgaggagcgcgaggagttcatggacatggtcctgcgactccaggccctggacgccacgcccgaggaactcggggccctcggcgccaggatgaacgagttcatgatgaagctcgccgccgccaagcgcgcgaaccccggcgacgacctgctcagccacctcgcccacgaccccgacgccgacccggcgctcacggatctggagatcgccggcatcggcgtgctgatgctcatcgcggggcacgagacctcggccaacatgctgggcgtcggcacctacaccctgctggagaacgccgaccagtgggccctgctccgtgacgacatcagcctgatcgaccgggccgtcgaggagctgctgcgccaccagaccatcgtccagcagggcctgccgcgcggcgtcacccgggacatggagatcgccgggcaccaggtgaagaccggggagtccctgctggcctcgctgcccgccgccaaccgcgaccccgccgtcttccccgaccccgaccgcctcgacatcacgcgcgagcacaacccgcacctcgccttcggccacggcatccacctctgcctgggcatggagctcgcccgggtggagatgcgccaggcgtggcgcggcctcgtcacgcgcttccccggcctgcgcatggccgccgcgcccgaggacatccgctggcgcgacgaccagatcgtctacggcgtgtacaacctcccggtgacctgggacgaggccaagtgaccggccccgaggccgcggtgcgcgggtgccccttcggcgccggcgaggcgcccgcgtaccccttccacgcccccgaccggctggagcccgacccgtactgggagccgctgcgccgcgagcggccgctgcaacgcgtcacgctgccgtacggcggcgaggcgtggctcgccacccgctatcaggacgtgcgcgcggtcttcgccgaccgcaggttctcccggcagctcgccgtcgcgcccggcgctccgcgcttcctcccgcaccagccgccgccggacgccgtcctgagcgtcgagggccccgaccacgcgcggctgcgccggctggtcgggaaggtcttcacgccgcgccgcgtggaggacatgcgtccgctcatccagcgcaccgccgacggactcctcgacgcgatggaggagatggggccgcccgcggacctggtcgaggacttctccctgcccttcgccgtgtccatgatctgcgagctgctcggcgtgccgcccgaggaccgcaagcggttctgcgtctggtcggacgcgctgctgacgaccaccgcgcacacccccgcccaggtgcgcgactacatgatgcagatgcacgactacctcggcgggctcgtcgcgcagcgccgggtgcggcccaccgcggacctgatcggctccctcgtgaccgcgcgcgacgaggaggacaagctcaccgagggcgagctggtgcggctggccgaggccatcctcatcgccggctacgagacctcggcgagccagatccccaacttcctctacgtcctcttccgccacccgcagctgctggagcggatcaggaacgaccacgacctcatccccgacgccgtcgaggaactgctgcgcttcgtgcccatcggcaccgtggacggctttccccgtacggccaccgaggacgtcgagctcgggggagtcctggtcagggccggggagacggtcgtgccgtcgatgggcgccgccaaccgcgaccccgagctgttcacggaccccgacgagctggacctcgcgcggcggccgaatccgcacctgggcttcggcgcgggaccgcaccactgcctgggcgcccaactggcccgggtggagctccagatcacgctcacgacgctgttccgcagatacccccgcctgcggctggccgtgccggaggagagcctctcgtggaaggaggggctgatggtccgcggcatgcacaccatgccggtcacctggtgaggacaccggcgtcctcctgaccttcccggcgttctcacgccgtcccggcagccttccttccgacacgagcgcacagagggtgaagcgaccgcaatgagcaccatcgacgaatgggaacacagcacgaaggaggcgggcatggaccccgcggccctcagacgcctgaccgatgtggtgcgggcgaggggcggcgcggcgcagctgtgcgtcatgcggcggggcaccgtggtcctggaccgctcgttcggctgctcctccgactccctcttcctcgtctacgcggccaccaagcccgtcgccgccctcgccgtgcacgcgctcgccgagcggggcctgatcgggctggaccggccggtggccgaatactggccgcagttcgcccggcacggcaagggtgacgtgaccgtccgtcatgtcctccagcaccgggccggggtgccggtcggccggggcatcgtgcgcacgatgcgcaccgccggcgactgggagcgctccgtgcgcgaccttgagcagtcccggcccaagtggcccggcggcgaggtcgccgcctaccacttcatgagtttcggattcattctcggcgaactggtgcagcgcgtcaccgggcggtcgttccgagatttcgtgacttccgagctcttcgccccacttgggctgaatgatttgcacatgggattgcccggcagtgcctggccccggcatgtgcccgcgcgggccgcccacccctccgaatggcccaatcagtggatgagcaaccgccgcggctaccgccaggccgtcattccgtccgccggtctttccggaaccgccgcacaaatggcccgcttttaccagatgcttatggagggcggctcgctcgacggcatccgcgtgctgcggcccgaaactgtggaggaagccagaaaaccgtccaatgacggcggaatcgacgcttccctcaagcgtccggtccgctggtcccacggattcatgctcggtggtccgggcccggacccgcgggggctgtccaatgtgctgggccgcacgagcgacccgagcgccttcgggcacgcgggcaccacgtccagcgtcgtgtgggccgaccccacgcgcgagctggtcctcgcctacctctccaacatccagcccgagttcggagcgggtatcgagcggctccgcgaggtcagtgacctcgcgctcggtgcctgcgaggcaggctgacccgagccgtgccgccacggcccggcgcccgcccgatccgatcgggtccggtgggggccggccgggtccgggcggggacgcacttcccccggcgtccccgcccgggccccggtgcgaaccgggcgcaaaggcggccgatcgcccggcgcggccggatgcccccgaacggtgtgaaacgttcttatcagcctctgaccagcaccgagtgatctactgcacagcccgaggccgcgattccggcagtatcttgatcttgacggggcaccaatgcgagcgggctattcgccgcggttttccctgacgtcggatgcagatgacaccggaggagggccagtgctgaatctgcccaaaggaatggagcgcgcgcatccgcattctccgccacaggtgggaatactcggacccttggaagtccgctcggccggaggtgccggaacgggagccgcggtaagcggtattcgcgtacgcacattgcttgccgcgttgactgcccgcctggggcaggcgatgtcgaccgagcgcatcctcaaagaggtctgggccgacaacccgcccgcgaccgatcgcaaggcggtggccgtcgccgtcctgcggctgcggcgggtcctcggcgacaacgaaggacggtggctgctcacccgcccctccggttacgtcctggacatccccccggaccacctcgacgccgtacgcgcggagaccctggtgcgggaaggccgggccgccctggccgccggcgacccacgcgtcgcggcccgccacctcacgcgcgccctcgaccagtggcggggcgagccctacgcggacgccaacgccatctcgaccgtgtcccagcgcatcacggagctggagaacctcaggtccgaggccgtccaggcgcacatcgacgccaggctcgaactgggtcaccaccaggaactggtcggcgaactccgctcgctgaccgccgcgaaccccctgcacgagccgcactggctgcagctgatgctcgccctctaccgctccggcaagcaggccgaggctctcgccgcctatatgcagctgcggcaggcgctggccgagaacctgggcatcgacccgggtcgtcagctccaggaactgcacctgcggatcctgcgcgccgacgcgggcctgctgacggggtccgggccggcggcaccggccgagccactgctcgtacggcagtcctgagggctcacggccacccgaagaacgcgcggtagcacggaacctgctgctcca

SEQ ID NO: 75 - DNA, 18034 bp, Streptomyces lavendulae
  unsure (302)...(302): n is a or t or g or c
cccaggacctcgtcgcggtcccggccgcgtggtggacctccgccaaccccaacaacgaccagctctgccagggcatatcggtggaggtcagctacaacggcaggaccatcagagtgccggtgcgggacaagtgcccttcgtgcgaccggacccacatcgacctcagcaggacggccttccagaagctggcgccgctcgacaggggtgtggtcaacggcatcacctggaagttcgtccgctgacgccacgccggggtccccaaagcccgggaccccggcgctccgcgcccggcacgccggggnccgcccggcgtgtcggcgtgaggttcgtcgccttcaagagtcataaagacaatcgcgactgttgacgttatgagttcatcaaatttaaggtcgcgggactcttggaacagatcaagacgacggagaacaatgacgtactcccccggcgcgcggccgcgcccggcccggctgtccgcactgctgctcgcaggcgcgctcgtcgcctcggtgccgcccgcggccgccgcgcgagcgccgcaaccccccaccgccgaccgcccccgcaccgccgcctcccccacaggcggctgccgtacgggtgacggctggacactcgactccacccgcatcgaccccgacgacacccaccacgcctatgtcggcaacggctacctggggcagcgcgtaccgcccaacggcgccggctacaccgacagcgacaccaagaccggctggccgctcttcgctccggcctacgacggctcgttcgtgtccgggctctacgcgcacaacaagcagaccgccgccgaccggcaggtgatcgccgctctgcccacctggaccggactggccgtcggcaccggcggcgagcacggcgatatcttcaactcttcgacgaagtcgggccggatttccggatatcaccagaccctcttccagagctgcggcatcgtccgtaccgccctgacctggaccgccgccgacggccgcaggaccgacctggtctacgaggtgctggccgaccgcgacgacccgcacacgggcgccgtacggctgagcatgacgccgcgctggagcggcgaggccaccgtcaccgaccagctggacggacgcggcgcgcggcgcatgcggcagaccggcggcggcgaccgcaccggtgggaccggccgggacggccgcaccatggacgtggccttccgcaccgacggcacggacaccgacggcgccgtcgcctccaccctgagggccgggcgcggtgtgcacacgaccggggaccgacgcgccgcggccgcgaaggacttgagcgtgaaccagtccctcacgttccccgtccgtgcgggccacgcgtacgaactcaccaaatacgtgggtgtcgacaccgcgctcacctcgcacgcgccccgcgaggacgccaccaccgcctccctgcgcgccgcccgccgcggctgggacgggctgctgcgtgcccacaccgccgcctgggcccggctgtggcgctccgacatcgagctgccgggacagcgcgacctccaggcgtgggtgcgttccgcccagtacgggctgctgtccagcacccggcagggggcatccaacagcatcgccccggccgggctgaccagcgacaactacgcgggcctggtgttctgggacgccgagacctggatgtacccggccctgctggccaccgcgccccaactcgccaggaccgtcgtcgactaccgctaccgcaccctcgccggagcgcgcgagaacgcccacaagctcggctaccaagggctcttctacccctggaacagcggcagcgagggcgacctggcccaggagtgccacagcgtcgacccgccccactgccgcacccagatccacctccagtcggacatctccctcgccacctggcagttctacctcgccaccggcgacaccgcctggctgcgcgagcgcggctggccggtgatggagggcatcgccgaattctgggccgggcgggtcacccccaacgccgacggcagctactccatcaaggacaccgccggccccgacgaatacagcaacggcgtcgacgacgcggtcttcaccaacgccggtgccgccaccgccctgcgcgacgccgcccgtgccgcgcggctgctgggcgagcgcgccccggcggagtggacgacgatcgccgaccggatccgcatcccgtacgacgcgcggcacaaggtcttcgagcagtacgacggctacccgggcagcaagatcaagcaggccgacacggtgctgctgatgtaccccctggagtggccgatgtcccaggccgacgcggcgcgcaccctcgactactacgcccggcgcaccgaccccgacggccccgccatgacggactcggtccacgccatcgacgccgcggccacgggcgagccgggctgctcggcgtacacctatctccagcgttccgtccggcccttcgtgcgcggtcctttcgaccagttctcggaagcccgcggcaccaaggccggcgccgacgaccccctggccggctcgcccgcccacgacttcctcaccggcaagggcggcttcctccagatcttcaccaacggcctgaccggcatgcggatgcgcgaggaccggctgcacctcgacccgatgctgcccccgcagctcggccgcggcgtcaccctgcgcggcctgcactggcagggccgcacgtacgacatcgccatcggcgcccacgagaccaccgtgcggctcaccgggggtgcgcccatgaccctctacaccccgcagggcgagcacgtgctgaccaaggcggcaccggccgtgctcaagacccgccgccccgacctcgctcccaccgacaacgtggcccgctgcaccaccgccggtgcctcctccgaggaacccggtatgtacgcggcagccgcggtcgacggcaaccccgccaccgcctgggtccccgacgggccgaacggtgaactgaccaccgacctcggcaagtccgtacgcgtcaccaaggccacccccgtctggagcggcccggcaccggcctcgtacagcgtccagctctccctcgacggccggcactggcacgacgcggtcgcgggcggcgctccggtgtccgcgcggtacgtacgcgtcgcgctacgcggtcaggccgatgccaagtcccgtacgggcatcgccgagctgaccgttacgtagggcaccagcagcccgcgcgcccgggctggatgacgacgaggatccgcgggacttcacccgccctcggccgacagggacgtcctgacgagagcgagcacgtcgtcgtcgctcagccccagcgcgcgggcgtcggcgaccaggcgccgggcctgcacggtgagccgggcgcgctcggcggtcgaccctccggtgacgaccgccccgcgcccccggcgcagttcgaggaggccctcctcgcgcagccgttggtagccacggagcacggtgtgcatgttgaccccgagcgacgcggcgagatcgcgggcggacggcaggcgctcgccggagcgtacggtgccgtcggcgatggcaccacggacgcatgcggcgatctggtcgcccagggggaggggggacgcggggtcgacgcggaagagcacggtcacccgcccgcggaggtgcggcgttcgcggtcggcgagggtgttgagcagcgcggcggccgtggcggcgtcgtcgacggtgacgacgaactcgctgccggtggtcagacggacgctgatggcgtcgccggaacgcagcacgacgccgctcgccccggaacggacccggtagccccagccaccgaagtcccgcagaggcttgaccggacggtgaccggcttcggcgatccgctggagcggcacgttgatgcgcggccaggggacggtcgagggcgtgacggtgagcccgcgccggtcggcggtcacccggacacccgtcagcagggtcatggcggctccgatgaggaacagcgacagcgcggacagccatccggcggcgaccccgacgacgacgccggaggcgaagaccaggacaccggtgaggggcagcacccgggagcccgccacccttgaccagccggcgatctcggagtcgccgagcgcgagacgcgaggcatcggcggacggcccggaatcgctgtcggctccctggtccttgccacaggccgcccagcccaccgccgcgtagagcgcagcagccccgaaagcgagcgcggcctgcgccccgggcaaggtgacggaagaggcgtcgtggacaccggtgttggccagcagcacggcggtggccagccatccggtcagcaccgcgacggcgccgccgatgacggccagcacgcgctgtccgccgttgcccggccgcgtgaagtagacgagcgcgccgaagaggacaccgtcgcccagcagcactccccacgcgacgccgaggaaggagccctgccccgagaagccgtcggcattccctcccggcccgatgtgcgaggcgatccgcccgggcagccggtcccgcaccgagaggaacacccacaggacgacggcagcgcagaccagaggcggcacgacggagacggcaaggcgacggacgaggacggacgaggaccgtgacagcggcatgagagcaaacctccacttgtttgcacactagtagaacaagtggaggtgaactcggcgaaggcggctgcctcttcctgacgcgttccgaacgccaccggagccgccacgactgacccagtgtcaccccgcgggaggcggaacgcttcagtccgtgccgggagcggcggccgcttcccgtggtgcgactggtggtgtctgcgtggggcggcgcatgagcggcaggcggagccgccggatgcccggccccgagggaggtgccagtctgcgcgtggacctgtggaagacgagcaggctgacggcggcggctccggcgaaccaggccagttggacggcgaggtagccggagacgggagcggtcgagaagccggccgcggcaccggtctgcagcgagccgtaggaggggaggagcgtcacgaggccgccgttggcggcgccgccggtggtgacggggttctgcaggcccgcgtcgagaccgctggtcatcacgatggcgaacatcccctcgacgtcccggcgcagcagggagccgaagacgatgccgatggcgccgtaggtcagggacgcgcagaacagggcggcgacgaagaggaccggccggcggggcgaccagaaggcgtaggtgatggcggtggcgtagacggcgacggtggcggagatgagggtgagggcggtgagcttggcgagaaggaggtggacgcgccggtagcccgccatggacaggcgtcggtcgaaggggccgctggtgaaggtcgcggcgaacatcatgaagccgacgatcagggtgatggagttcagcgccccgacgatcgacgtgagttcgttgccccgcgggtggaggatctgcccggtctcgtgcagcctgaaggggatgggcgggtcttcgatgacgcagtaggccagcgtgatccacaccgggatgaacgcggcgatcaggcccatggcgagccggttgcgcaggtgctcgaccaaggcgaaccgggtggcggtgacgtagagggttgtgtggttcataccggtgccgtcgtccttgagtgcagcagtccgccgtcgaggtgccggagttcgtcgagccgttcggcgtcgtaggccaggtgggagacgaccagcacggaccgcccgcgttcgcgcagaccggcggccaggctccagaaccgctggtgggtgtcccagtcgaagccctggtagggctcgtccaggaggagcaggtcagggtcgtgcatcagggccagtgtgaggttgagcttctgtttcgtgccgccgctgagcacgctgacccgctcgtccccgtagtcggacagccggagcacgtccatgattctctcggcatggctgagggtggcgaggccgtacgccacccggaaatactccaggtgctggcggacggtgagagcctggttcaggacgagatgctgcgggcagtaaccgaaccggccgccgtagtggacctgcccgcgctgcggccgcagctcaccggcgaggatcttcaggagcgtcgacttccccgcgccgttctcgcccacgacgccggccagcgttccggggcgcagggacaagtcgatgccgcgcagcaccctgcgggtgccgtaggtgtggtggacgtccctgacgtccaggagattccggggcacggcttcctctcctcaggcgacctggtcgcgccggacgaccgagagggccagctcgtacaacaagtggctgtggcctgccgacggcagcaggggctcgagcttgttccacgcgtcgctcaccaactggtcggcttcctccaggcaggctctcaccgctccgcaggcgttcaggtcccggcacacctcggccaccgccgtcgcgctgcccgagccgtccttgacctgctgccagagctggttcagccgggctctgcggcagccggaccacggcgtgggccagtggcatggtgaccttgccgctccgcaggtcctcggtggcctgcttcgtcggtgcccccgcccgtgtgacaccgctcaggtcggcgacgtcgtcggcgatctggtaggcggtgcccaccgctgaaccgaaagcccccagtgctctccgcagttccggctcggcacccgtgacgacccctgctgcctccatggccgccgagaccggggccccggacttcaaccggtgtgtcagacggaccagttccagcacagtgtgccggtcgtcgccggccacggcctggtccatctcttcccggtgaccttggagatccagtgcctgaccggcgtgagccgctcgcagcgcggccagacccagtgcccgcaactccccgcaccgcgaggcgtcgtcgggaaaggtgagctgaacggcccgctcccagaggaaataggcggccgtacccgcgttcaccgcagtcggcatgccgaacatggtgtgcacggccggttgtccgcggcgcagcggtgaggcgtcctggacgtcgtcgacgatgagggatccggtgtggagcagctcactcgccgcgatcagcaggccgcaggattcgctgtcacctcccatcagaccgatggcctcccaggccagcaccggccgccagcgctgtcctccggcatcggtcagatggcggacgggagaggtcagggcccggtgcagccgctgggcgacgaccggcggcgtgccaggcccggtcgtcccggtgatgtggcccggtggcagccacgtggagcaagcatctgccgacgcgttcgggcacaggcggtcgatgtgatggttgatgcgttcggccgtgcggtcgatgcgctgccggatgaagtccgcgttcgccgcgaaagtcggggagatgtccctgggacagaggagggcgccggtcatggagtggtcagctttcggtcaggggcgggcgatggacgaggctccctcatggggtcgccggcccgatgccgcggagggactgctcgggcacggctcgcggagtgcggcgatcatggtcaggccgcgtggcatcgcggcgagttcgaagccggagcgcaccgtccggcccggacgcgccgtgcgtacggtccgcgtggcgaccagagtggcgagcgcgacgggcaccatgacgtcggccaccgcggcaccagggcagtagcggggcccgagcgcatagggaagccaggcagcaggggagaccgagggctgagcgtccggcatccagcgcgtcggatcgaagacatcaggccgttcgaagtgggcggggtcacggctcatcgccccgaggtggaggaacaccgtcgccttcgcgggcagccggtggccgcccagccacgtctcgcggcgcgtgcaacgcacaaggaccgggaggccgtgaagccggatgacttccttgacgaaggcggctgtccggggcaaccggtttattccgacgccggcctgcgagtgccccaggccgagtgccgcgtcggcctcctcctgcaaggcctgttgatggtgggggtggcggcccagttcgtagcaggcccacgcgagtgtcgaggcggtggcctccccgccggtgaacagcagcgtgcgcacgtcctgcaccgccgcttccgagggctcgcgccacgcttgcttcagcagggagaccacatcacacccgtcctcagccggccgatggcgcgcgaggacttcccgggtggcctcgtccagcaccgcgagggcacggcggagcgcgcgttgtcttggcacgggtacccaaggccacggggacaggagaaaccgcaaggccccgacctgcgacagcgtggcatgcgccgcggccagcgcggtcagcgttccgggcgagacctcgctgcgcaagacgcacctgacggcgagatggaaagtgagccgggtcatctcgtggctcatgtcgaccggacggtccgcgggaagggtggcgagcagacttcgggtctcggtccgcacggacgccccgaggtccgccggccggggcacggcgaacgcgggcctcatcaccgcccgccggtcacggtggaccgttccctcggtgctcagcagtccctctcttacgatcacccgcacatggggttccctgccccagaacatgaaggtgtcctcgtcgcgagcagcctgccggatcagcgaggagtcattgagcaggtacccgacgaacgggcccgccttcaccctgaccacaggccccgccttgccgagccgggccgcgatctccctcagatccatcaagaaccgcacggagcgggcatgcccgagcaaccgactgcagtcaggagccatcggcacaacggagtccgccttcgaatctccgttcatcaggcgtcctcccgcccgcatgtcaccctctgtcctcctgtgaacgaccaggagtgaggagtgtcacgcagagcatcacctgctgtatcggcagcaatgccgacccgcaccgacggctgggcggggcgaccgggaaccgccttgcggctacgccgtgctcgcgtgcctgaagcgcaccgtcacattcaccggtacccacgacagaggcgggttcgcggccacctgtatggacgcccgctgaatggggacggagtgcagggggtgctcgatggcggtgccttcgcggctcagcaggccctgcctcgcggtttgagcgcaacgggcggcagttcgggagcaggcagtcgggccccctcgtagccgtgcacctcgccgaagcgggagccggtcgcccgagccgtcgccgccgccggcccgattcgccatccggatggaagaggacaccgcgcagggaccgccgcccacttcaccggaatcctctccaccggaaaatttatccgcaaacctgtcacatcttcgacacatgaagcgtcagggcggtgacggcagttgaagccgttgcccgacgacgccgaaggagaccgtgggacagctacgcacgtgcgggccctggagcggtcggccacgccgacaggaatcggccttccgccgctgatccggcctcccagcgttcgcgaacacctcttgccacacctcccgcgcggcccccgcattcgagcggtcggctgacgacgccctgcgacgccgcgcacaccacccacgcacctcgccacgccgaaggctgcccgaaaacaagaagaccgaggaaagcacacatgaagatctctcgaataggccgcgcgtcatccatcgccgccctggtgacaaccgcactcgctttcacggcagttggcaccgtcgctcccacggccgtcgccgactcccgcgcggccgccgcttccgggacgcagaatgaccacccgagctcggggcagggcacctccacctctgagctccggcgcaagggcctggtcccgtcgagtctcgtggccaagcccatcacccgcagcgagaccctcagacgcgccgccagctggttcggcaagggtctccactacagcggggacaacacctatcagggctggcgcacggactgctccggcttcgtctccatggcctggggactgcccggcccgggtgagaccaccgattcgttcattcccgggggcgtggcccacgaaatctccaaggacgaactgaagcccggcgacgcgctcaacaacaaggcgctcggcaacgacggtcacgtcgtcctgttcgagaagtgggccgattcctcccagtcctcctactggggttatgagttcagcagcagcggtctgcaccaccgtgtgatcccgtacgcctacttctccaggtccgagcagtaccgcccgatccgcttcaacaccatcgtggacgacgacacggccgcagggcccgccgaggacaacgcccgggtccagggtgacttcgacggcgacggccgcgacgacgtggcggtgctctacgactacggcaggaaggacgaccgcagtcgctcggccctgtggacgttcaacagcaacggcagcggtttcaacagtcccaagcaggtgtgggacagcgggacgtcggagagctggaactgggcctccagcaagttgacggtcggtgacttcaacggcgacggcaaggccgacatcggcgtcctctacaacatgggcgcgaccgaggacggccgcaaccgcaccaagctgttcgtgttcaccagcaccggcagcggattcgccgccccggtcaaggtctgggacagcaacgacgaccccgtgaagagctggaactggaacgccagcaagctcaccgtcggcgacttcaacggcgacggcaaggccgacatcggggtgctgtacgactacggcaaggacgacgaccacaaccggacagggctctggacgttcaccagcaccggcagcgggttcaacagcccgaagcaggtgtgggacagcaacaacgaccccgtgaagagctggaactgggaagccagcaagcccgtctccggggacttcaacggcgacggcaaggccgacatcggcgtcctctacgactacggcaagaccgactccggcagccgcaccggactctggacgttcaacggcaatggcaacgggttcaacagcccgaagcaggtgtgggacagcaacaacgaccccgtgaagagctggaactgggaagccggcaagcccgtttccggcgacttcaacggcgacggcaagagcgacatcggcgtcctctacgacatgggtcgcaccgaggacggccgcaaccgcaccaagctgttcaccttcaccggcacggcgaccggtttcaacagcccggtcaaggtgtgggacagcaacgacgaccccgtgaagagctggaacgcgtccgcgagcaagcccgtcgcaggtgacttcaacggcgacggcaaggcggacatcggcgtcctctacgactacggcaagaccgactccggcaaccgcagcggactgtggaccttcaccagcaacggcagcggcagcgacagccccaagcttggctgggacagcagcgcggaccccgtcaagagctggaactggagcgcgagcaagctcggctgaccggcttcgcccctcctcacctcaccgttcgggagagtcaccgcacatgcgaaccatacgaatacgaagaacgaacggcgtggccttcgccgccgctgccgccctgatggccctcgtcgcctccggcaccgccacggtccaggccgcgccctcgcacgccggaccctccggcaccactccgatcacctaccgtggcctcaccctcgacataccctccgggtggccggtcgtggacctggagaaagacccgcacacgtgtgtgcggttcgaccgccacacggtgtacttgggccaccccggcaccgaacagtcctgcccctcccatctggtcgcggacaagacggacgccctgatattggagccgatcaccggagcgggcggccaggacgcctcccacgcgctgcgcatccctgccggggccccgatgccgcacgagctgccggtgacgtacgaccacgagacgaaggtcgccgtcgaaggcgccggagtcatggtcacgtcctcctacggcacgtccagtacaacggtcgccgccgtcctcggctcggcccgcacggacgcgacagccaagccgacccccctgcccggcaaggcgggcaggggcctcgcggctccaccggttgccgccgtcgcggccgacaagggatacacagggctgggcttcgagtcctgcaccgccccttcgtccgccgcgatgaaggcatggaaggcctcgtcgccctacggggccgtcggcatctacatcggcggtcgcaagcggggctgtgcgcaaccgcagctcaccggcgactgggtgcgtcagcagaccgccgacggctggcacctgctgcctctcttcgtggacctccaggccggcgacatctctccggccaccgcggacgcgcagggccgcgagtccgcggacgccgccgtggccaaggcggcggacctgggcctgggccccgggacggtcatctacagcgacatggagcactacgacagccgctcgtaccgggcccgggtcatcgactacgtgtcggggtggaccagccgcctccacgaacatggctaccgctccggtgtgtacgcgggtgaaacgagcggcatcccggacctcgcctcggtggccgacgacaccaactacgcatcacccgacgtgctgtggtcggcgaactggaacctcaaggccgatgtgtcggacgcgtcgatgggacttccgggccccggctactggcccaatgggcggcgcatccaccagtaccgcggccaggtgaacgacacctacggcggtgtcaccctcgccatcgaccgcgactacgtcgatgtcgccgcggactcggccctgcccgcacccggcggagaggacggttcctcgcgcgtcaagggcgacttcgacggcgacggccgcgacgacgtggccgtgctgtacgactacggcaaggagggcggcgtcagccggtccgcgctgtggacgttcgcggggaccggcagcggcttcggcgccccgaagaaggtgtgggacagcggatcggacagctggagttggtcggccgccaagctgacggccggcgatttcaacggagacggcaaggccgacatcgcggtcctgtacgacatgggtcgcactgaggacggccgcaaccgcaccaagttgtacgagttcaccagcaccggcagcggattcaacagcccggtcaaggtctgggacagcaacgacgaccccgtcaagagctggaactgggcctccagcaagctgaccgtcggcgacttcgacggcgacggcaaggccgacatcgcggttctgtacgactacggcagggacggcgaccgcagccgtacgggcctgtggaccttcaccagcaccggtgccgccttcaccggccccaagctggtgtgggacagcaacaacgacccggtcaagagctggaactggaacgccagcaagcccaccgtcggcgacttcaacggcgacggcaaggccgacatcggcgtcctctacgacatgggtcgcaccgaggacggccgcaaccgcaccaagctgttcaccttcaccggcacggcgaccggtttcaacagcccggtcaaggtgtgggacagcaacgacgaccccgtgaagagctggaactgggacgccgtcaaggtagtgggaggcgacttcaacggcgacggcaagagcgacatcggggtgttgtacgactacggcaaggacggcgaccgcagccgcaccggactgtggaccttcaccagcaacggcagcgggttcaacagcccgaagcaggtgtgggacagcagcaacgacccggtgaagagctggaactgggccgcgagcaagccggtcgcaggggacttcaacggcgacggaaaaacggatatcggcgtgctctacgactacggcaggaccgattccggcaatcgcaccggactgtggaccttcaccagcgacggcaccggattcggtacacccctcctgggctgggacagcgtgacggatgccgtgaagagctggaactggcgtgccagcaaggtgagttgacacccctcctgtgagacatggggcactcctcgacgcccgtccggcccggctgcggcccggccggacgggcccgtcattcaatggaaggaagaagtggatcccttgacgcgcaagacccgcaccccccgcaagaagggcagacgcgcgagcgcggcggcgatgtcggcctccggcatgctgctcgccttggtggccaccgccgcccccgtccccgcccaggcggcatcactcgccacctgggaaaagatggcccagtgcgagagcagcggggactggggatacaaccagccaccgtactacggcggcctgcaattcctggagagtacgtgggtggcgtaccacggaacggactatgcgccatacccctatcaggccaccaaggaacagcagatccgggtcgcgcagcggctcctcgacaatgagggcgcggctccctggccgtactgcggaaagaaggtggggctggctgacgacgacgcacgccccttccccgacgcgccggacgacgacgcctccgcccggatcaacggtgacttcgacggcgacggatgcgacgacgtggccgtgctctatgactacggcaaggagggcggcgtcagccggtccgggctgtggacgttctccgggagcggtaccggcctcggcagcccgaagaaggtgtgggacagcggatcggccagctggagttggtcggccgccaaactggccgtcggcgatttcaacggcgacggcaaggccgacatcgcggtcctgtacgacatgggccgcactgaggacggccgcaaccgcaccaagttgtacgagttcaccagcaccggcagcggattcaacagcccggtcaaggtctgggacagcaacgacgaccccgtcaagagctggaactggaacgccggcaagctcaccgtcggcgacttcaacggtgacggcaagaccgacatcggcgtcctctacgactccggcaagaccgactccggcaaccgcaccggactgtggaccttcaccagcaacggcactggattcaacagcccgaaacaggtgtgggacagcaagagcgacccggtgaaaagctggaactgggccgcgagcaagccggtcgcgggcgatttcaacggtgacggcaagaccgatatcggggtgctttacgactacggcaaagatggcgaccgcagccgcaccggactgtggaccttcaccagcacgggcagcggattcaacagccccaagcagacctgggacagcgggtcggaaagctggagatggtcggcggccaaggtggtcggcggcgacttcaacggtgacggcaaggccgacatcggggtgctgtacgacctcggcaggaacggcgaccgcaaccgcaccgaactgttcacgttcgcgggcaacggcaccggcctcaacacaccggccaaggtgtgggacagccaggacgacagcgcggtgaagagctggaactgggccgcgagcaagccggtcgcaggtgacttcaacggcgacggaaagacggatatcggcgtcctctacgactacggccagaccgactccggcaaccgcaccgggctgtggaccttcaccagcgacggcagtggattcgccggccccaagctcacctgggacagccggaccgaccccgtcaagagctggaactggaacatgagcaagaccggctgagccattcatgccgtacagaagagaagaggaaggatgaaataccgaccgggaacactgctcacttccataacagtcttgtgtgccctgctcgttccggtgcgttcggcggctcaggcggccaggcccgagcagggacgttccgtggtggccgcggccgccgtactggagcaaagtccgccgacgctgctcgccgagccggaaatgcgcgtcgtctcctggaacatctgcggtgaggcgggcggggtgcgcggggagggcggctactgcccctaccgcaacgatccccaggcgaaagtcgaccagatcgcgcaggtggtcgcggagcgcagtgccaatgtcgtcatgctccaggaagtgtgcggcgaggcgcccggcagccatatggagcggctgcgcgcggccctgggcagcggatggtcgatcgcgcacgccccgggggcccgcccggacgacggaaccacgaactgccggggcgggctcagcggcatattgggcgtggggatcgcggtgaaggggcgcgtcaccgacaccaccgcgacgaacaccgtgcccgggggcggcggtgacaagcagaccctgcccatcctctgtgtacgtgtcgagggctggtcgtccaggatctgcaccacccacatcctgtccgaccctgccgatccgcgcaggccggggcagatccagaacgtcaagaacgagatctggccggaccgctatcagctggtgctcggcggcgacttcaacatgttccccgactccgccgggctcaagccgatctcggacgaattcgacgagtgcgaccgccgctcctacggcgccggtgacatggtcaacgaggtcacccatcactcctgggagaaaaagggcggacacatatggcgcaagcgtgaccacatcttcgcctcgtggggagagtccgggagccagttcacatcctgcgaggtcgaccggacccggatggacaccaccgagaacgcgcccgaaagcggtccgcccaacgggtattcggaccatgcgccgatcatcggctacctcaagccgccgcggcacctgagcacgtccggggacttcgacggcgacggcaaggccgacctcgcggtcctctacgggcaggggaagaccccggacggccacaaccggtccagcctgtggatctcaggcggttccggtaccggagcggagaccggattcgccgcgccgcgcgaggtctgggacagcggtgccgacagctggaactggtccgcgagcgcgctgacctccggggacttcgacggcgacggcaagaccgacatcggcgtcctctacaactacggcagggacggcgaccgcaaccgcaccgcgctgtggaccttcaaggggacatcgaacggcttcgaggcgccccgcaaggtgtgggacagccacgacgacacggccgttcccagctggaactggtccacgagcaagctcgtcgcgggcgatttcaacggcgacggcaaagcggacatcggcgtcctgtacgactacggcaggaccgcctccggcaaccgcaccggactgtggaccttcaccagcaccggcaccggattcggcaagccccacctggcgtgggacagctccaccgacccggtgaagagctggaactgggccgcgagcaagccggtcgcaggtgacttcaacggcgatggcaagaccgacatcggcgtcctctacgactacggcaaccacaccgccctatggaccttcaccagcaacggcaccggattcgccggccccaagcaggcctgggacagcggaccggagaactggaactggtccgccgccaagccggtcgccggggacttcgacggcgacggcaggaccgacatcgcggtcctgtacgactacggcaggaccgcctccggcaaccgcaccggactgtggaccttcaccggcaccggcaccggattcggcaagccccacctggcgtgggacagctccaccgacccggtgaagagctggaactgggccgcgagcgagccggtcgctggtgacttcaacggggacggcagggccgacctcgcggtgatgtacgactacgggaacgcgaccaacggccgcaaccgcaccgcgctgtggtccttcaccagccgcggcacggacttcgccgccccgcgggcgaactgggacagcagcaacgccgctgaccagctgaaatcgggcgagctgagggcggctccgctcagcgggtcctagttctccatgatcggtccgtcgccctccagaccggccgctctcccggtcagcgtcgcggccagtgcgtcagcgtcgcgaccgagtccgtaacagcgcatcccggcgatcgcgaagtacgcctggtcgagccagacgcgggccgcgccagtgctgccgcgcggcgaagtacggcgagctc

SEQ ID NO: 76 - DNA, 53500 bp, Streptomyces lavendulae
gtccgggccggcggcaccggccgagccactgctcgtacggcagtcctgagggctcacggccacccgaagaacgcgcggtagcacggaacctgctgctccagcatatggatgccgtggtgcacacggcgcccggcggtggcggccgcgctcagcagcgccgtctcgtgcggcttcatgacgacgtcgaccaccacggcatccggtcgcaccctcgcggggtcgaagggcagcgggtcctcggaacgcatgcccagaggcgtcgcgttgacggcgaaatcggccgcctccagatcgccgggccccagcgcccggatcccgtccggccggcgggacccgagccgcagcagcagcgcgtcgagctgggcgcggtcggtgtcgtgcacggacacccgcgcggcgtcggccatcagcagcgccgtggcgatcgcgctgcccgcccctccggcgccgaccagtgccacatgcctgtcgcgcaccgtgtgcccggccgcctgaagaccctggacgaacccgagcccgtcgaagttctcggcgtaccagcggccgtcgggttcgcgccgcatcgcgttggccgtcccgatgagggcggccgccggcccgagcccgtccgcgagcccgcacagggccgccttgtgcggcacggtgaccagcagaccgtccagattgccgatccgcttgagcccctcgaccacctcggcgagatcccgcgcccggacgtgcaccggcaccaccacggcgtccagaccgctttcgctcagcagggggttgagcagaccgggcgccttgacctgggcgacgggatcacccagcaccgcgtacagccgcgtggcgcccgagacaccggccgccggcccgaggaattccatcagccgatcctctctgtacccccgacggatgttgccctacggtgctggagatgctccacagctttgccgtgaccgccggtcggcacaaccctgcgtgcccctgacgcgccaggccctccaggtagttgctcccggcggatcccgacagctcccgaccggtcccgacggagggaagaagccatcagatacctgggaatcgacgtcggaggcacgaaggtcgccctgcgggtgacgggggacaccgacggtgcgggcggcggcgacgtgacgttccgctggcccgccgccggcgacgtcaccgcggatctggacctgctcgccgcgcgggtccgcggtcttctgggacaccgcgaggaccccctcgccggggtcggcgtggccatgcccgcgatctgcgacgcggccgggacggtccgcacgtggccgggacggccgagctgggcgggcctgaacctgacggccgccttcgggcagttgctgcccggcaccccggtcgcctgcgccgacgacggtgacctggccgcgctggcggagtcccgcgccgccggctgccggcatctgctgtacgtgggggtcggcacgggcatcggcggcggcatcgtccatgagggccgcgcctggccgggccccggacgcggctcgtgcgaggtcggccatgtcgtcgtcgaccgctcgggcccacgctgcgactgcgggcgcgccggctgcgtccaggcggtcgcgtcgggaccggcgaccctccggcgggccgccgaacggcgcggccgggagaccggcttcgacgaactggcctccggggcgcgcttgcacgccccgtgggcggaagcggccgtcgacgagagcgccgcggccctggccaccgccgtgaccggcatctgcgagctggcccaccccgaactcgtcctcgtcggcggcgggttcgcggcgggcgtgccgggatacgtggcctcggtggcggcgcacgtcgagcggctgacccgcccgggaacggatcccgtgcgggtgcgcccggcggtgctcggcgggcggtcctccctgcacggcgcactgctgctcgcgcgggaggcacacgggcggggaaaccggccgccggagagtgaccgtgtttcttccgatgtttcttccgatgtttctttcgggggagtgacagacagggccgttggccggtccgactgagcacaatcacaggtgatttcgcccaggttcaccacgcctcgtgtgctcggggtcggcagaaggagtcagagtcatgctcgacaggcggagcgtcattcgcgtcggcgccggggtggcggcggccgccgccgtggccggtacggccgccaccggtgcggcggccgtggggctgccgggtgtacggggacgcgcggcgtcgcgcggggtcgactgggcctccttacgccgtcatctgtcgggcgagctcgtcctgccggcggacaccggatacgagcgggccaggaagctctacagcggccagttcgacggcatccgcccgcaggccgtcgcctactgccggaccgaggaggacgtgcggacgaccctcgcgttcgcccaggaccacgcgctgcccctcaccccgcgcagtggcgggcacagcttcggcggctactccacgaccgacggaatcgtcctggacgtctccggcttccacgcggtgagcctcacccggaacaccgtcgtcatgggcgcgggcacccagcaggtggacgccctcaccgccctgtcgccgcgcggtgtcgccgtggcgagcggcaactgcgcgggcgtctgtcccggcggcttcgtccagggcggcggactgggctggcagagccgcaagttcggcatggcgtgcgaccggctcgtctccgcccgggtcgtgctcgccgacggccgcgccgtgaccgcctccgccaccgaacaccccgaccttttctgggcgatgcgcggcggaggcggcggcaacttcggcgtcgtcaccggcttcgagctgcgccccaccgacgtcccctccgtcgtcagctacaacctcacctggccgtgggagtcggcgcggcgcgtcatcgaggcgtggcagcactggatcatcgacggcccccgcgacctcggtgccgcgatggccgtgcagtggcccgacgccgggaccggcacgccggtcgtggtcgtcaccggcgcctggctgggcgcggccgacgcgctcacccccgtgctggactccctggtggcctccgtgggcagcgcgcccgccacccgctcggccaaggcgctctcccagcacgacgcgatgatggcgcagtacggctgcgccgacctcacgcccgagcagtgccacacggtcggctactcgcccgaggccgcgctgccccggcagaacttctccatggaccgcaaccggctcttctcccgggccatcgggcaaggaggcgtcgagcggatcctggaggcgttcgccgccgacccgcgcgccggacagttccgcttcctgagcttcttcgccctcggcggcgccgccaaccgccccgaccgcaccaccaccgcctacgttcaccgcgacaccgagttctacctcggtttctcgatcgggctgaacgacccggagtacacggcggaggacgagaggctcggccgcgcctgggccgcgcgaggactgcgcacgctcgatccccactccaacggcgagagctaccagaacttcatcgacccggagctcgacgactggaagtcggcctactacgccgagaactacgtgcgcctggccgccgtcaaggcggcctacgacccgcaccggctcttctccttcgcgcaggccgtctgacctctcccgaaagacccctgccggcctgctcccctccgcggctcctgtgggcactggtgcgcccgcgcacttctgtgtgattgagtgaagtccgggcgtgcagagctcagttgccgtggagggggcgccagttgcgagcatcagcggtggagagggtggagctgatccgctggccggtggagtccgagcggcgggagcgctgccgcgaccggggcgtcatgcggatcctggtgctggaggcgggggccgaggcacccttgtgcgtggaccccaaggaggactgggtccgcgctcccgtcagcaccgacgacctgcgggcccgcgtcgaggccctgcgccttcggggagccgccgccgagtcccggcccgaggtcgacccgaacggagtgctgcgtttccggtggcgctccgccctgctctcgcccaccgaggcccggctcgtcgcccggctcgccgagtcctatgccgaggtcgtcgcccgcgacgacctgctccgcccgcccccgggccgtaccgtgccgagccgtaacgcgctcgacctccacatcatgcggatccgacggcgcctcgccgcgctgggcctgagggtgcgcaccgtccgggggcgtggctacgtcctggagagcgcggaaggagtctgaccgacgggcgtggccgcgcaccgcaccgaccgcccctacgagcgaggagcccgaagtgcagcagcctcatcacagccgcgtcgacgtggaactgggcgagaggtcctaccccgtccacgtcggaccgggggtccgccacctcctgcccggcatcgtcgcctccctcggcgcgcaccgcgccgccgtcgtgaccgcacggccccccgacctggtgcccgatcccggcgtgcccgcgctgatcgtgcgggcacgtgacggcgagcggcacaagacgctcgccaccgtcgaggacctgtgccgcaagttcaccaccttcggcatcacgcgccacgacgtcgtcgtctcctgcggaggaggctcgacgaccgacaccgtcggcctggcggcggcgctgcaccaccgtggggtgccggtggtgcacctgccgaccaccctcctggcccaggtggacgcgagcgtcggcggcaagacggcggtcaacctgcccgagggcaagaacctcgtcggcgcctactggcagcccaaggccgtgctgtgcgacaccacgtatctccagacgctgcccgccgaggagtgggtcaacggctacggcgagatagcgcgctgccacttcatcggtgccggcgacctccgcggcctcgccgtccacgaccaggtcaccgcgagcctgcggctgaaggcgtccgtcgtcgcggccgacgagcgggacaccggcctgcggcacatcctcaactacggccatacgctgggccacgcactggagaccgccaccggcttcgggctgcggcacggactcggcgtggcgatcgggacggtcttcgcgggccggctcgcggaggcgctgggccgcatcggcgccgaccgcgcgcgggagcacaccgaggtcgtccgccactacggacttcccgacagcctcccgggaaacaccgacatcaccgagctcgtcgcgctgatgaggcacgacaagaaggccacgtcgggactgaccttcgtgctcgacgggccttccggcgtggagctggtgtccgggatcccggaggacgtcgtcctgcgtacgctcgcggcgatgccgcgaggaacggcctgaccgagtgttccgtcttccgaggggaagtgaccgtttcgtgtcggcagagctgtcagaaccgctgaagaaggccctggactccctggtgttcggcgtcgtggcgacgaccgaccccgacggccgcccgcaccagtcggtggtgtgggtccggcgcgagggctccgacgtgctgttctcgatcacgcgcggcagccgcaaggagaggaacatcctgcgcgacccgcgtgtgagcgtgctgatcagcccggcggactcgccgtacacctacgccgcgatccggggcaccgcgcacttcgaggacgtgccggacccgggcgcgtacctcgacacgttctccataaagtaccacggcgtgccctaccgggagtcgttccccgagccgccggaggtgagcaccattctcgccgtccggctcgttccgacgtcggtctacgagcagtggtgagggcgtaggcgtcccgaagccccggcagcgtcccgaatgccgctgccggggcttcccgtgggagccctacgccggtttccgcgcggtgaccaccgagtagccgacctcctccaccgagcccatgcggtcgatgccgtcgaccatgcggtggaacgcctcgtcgtccatgtgggagccgagctcgtccctggccgcacgcatcttcgccgccaccgcctcgtaggagggccgcacctcgtccccgatgtcgaggaactccaccacctccagccccaccgaccgcatgcagtcctcgtacgcctcgcgggtgaggacggggccctgctggaagttgtcgttggcggtgtcgacgatcgtcctggacgcgtcgctcaggggccggcgcagcacgaagtccgtcaccgtcacccggccgcccgggcgcagcacccgggcgatctcggtgaagacgtcggcccgttccagggcgtggcagatgctctcgatcgcgtaacaggcgtcgaacgagccgtcgggaaaggggagcgcgagcatgtcaccgatgcggaactcggtcgcctcgtcgccctccttctcggcgagctgccgcgacagacccacctggtaggggttgatggagaccccggtggcccgcaccccgtgccgggcggcgatgcgcaaggtggccttgccgttgcccgaccccacgtcgagcacccgctccccgggggcgaggcgcaggcgctccgagacgtagtcggtcagccggtcgcctgcctcttccaccgtcgtggggacgtcgggtcccgcccagtagccaccgtgcatgtagccgccttccgcgtgcaccatcaagtcggtgacgcggttgtagagttcgaccatccggtcggaggcggacgcggtttccgtcatgccgctcactttcccgggcgctgggcgaccagcagcagatagccgaactccttgacgccgacgaggtcgccggggtcgaactggttcaccatctcctcgccgaactgcgtctccagcctctgcttcgaggagttgatgcgctccgagagcagcctgaaggtcttctccagggtctggtcgctgatgtcgaggaactcctccagccacaggcccgccccccgcagcaggggagggtacgcctcggcgctgaccatggtcatcatgaagtcgtggaggtagcgctggacggcggcccgcccctcgggggcgaggggggcccgctcgaagaagtcggtgagcaccagacggcccccgggccgcagcacccggccgacctgggcgagcacctgggcgcggtcgggcatgtggatgatcgattcgagggcgatgacggcgtcgaagctctcgtcctcgaaggggaggtccatcgcgtcggcccgctggaagcgcgcccggtcggcgagcccggcctcctcggccagcgcgttggcccggacgacctgctcatggctcaccgagatgcccgtgacatgcgctccgctgagccgggcgatgcgtacgcccggggtccccacgccgcagccgaggtccaggacgcgggagccggcgccgatgcgcagccgctcggccatcatgtcggtgagccggtcggtggcctcggccagcggcacctggctgtcgggggagtcccagtagccgaagtgcaggttctcgccgagggaggcggctcccagcgcggtgaaccggtcgtagagcgcgcccacttcctcggaggcgggtgagggcatggggagttcggacagctcggagtgcggcatggacgatccctctcgtgaaaggtcgggggtgggtcgggcagtcggtgtcaggagagacggaggtcctggtagacggcgtcggcgaggcggacgatgcgctcgcggtgccgttcgtgctccaccttcccgttgaggtggagggcgacgacgagagcggcgtccgggtcggcgaagaccacggcggtccacagcccgtcgtgcccgaaggaccggggggaggcgtacgagccgaagctggtgaaccgcggatccagctgacggcattcgaggcggaaccccatgccccagtcggcgttgccgtagcggtcctggaggccggtgcggtgccgggccgtgagggcggcgacggtgggcggcgccaggacgcgcccgccgggagcgtccccgccgcgcagcagcatctcgaagagcctgcccatgtcccgcagcgggccacgggtgttgacccccgggatgcagcgtgtggtggccgcctccgtcgaccaccagtgggtgggcagcgggccgccctcggggttgctcacatggatcagcggcagctcgcccccgagcgcggcgaactcctcgcgatccaggtggacacgggtgccggacatgccgcacggcccgaggatctcctcctggacgtacgcgcggtactccctgccgtcgacgaccggaaggatgcgcgccaggacgaaccaggcccaccactggctgtagttgatgccgggcgtgccccccggacgcggtgccaccggcacctcgaaggcacggcgcacacgctcctcgtccgggccggccacgatgccgtgcagcgggtcgtcgccggtgggcagcgggcccgtatgcgtcagcagttccatcgaggtgatggactccttgccccggttgccgaactccggcagatagtgcgcgacgggcagatacgggtcgtacgctcccgcctcccacagccggcccagggcgaccgacagcagtggcttggcgcagcagtaccacaggggcagcgaccggtgggtcatcgccaccccggggcgggccagccccaacccggcgtccgccagagggactccgtcgcgggagacgtagatctgcgcccccggggtcgaggtgccgacctcgcgctccagctcccgcatggtgccgggaagcgccgcacgggcgcgccggacggcctcggccgcgtcctcggtgccgggcggcggggccgcttcccgcgccgtggtaccgggcgtgcccctgagcgcggccacatggcccgaggcgtcccgctccagtgcggcgtcgatgaagaacagcaggctgaaattgccctcggcccgtacgtcgccccgctcgaatacgccggtccgatcccgcgcggggccgagcagcagggcgcgggcggcctccaggggaagccggatccgcagtcccggactctcaccggccgcggccgcgaggccgagccgcccggcgacgaggtccacctggacgtcgacggccgggggttcgccgggcggtgggtcggtgagctccaggcggagcgcgaccgtgccgcgttcgcccacgggcgggcgttcgagcctcgcggccccggcgagccaggccgtggacaggaacggcgcggtggacagggtgttcagcacgggctcactcctccgcttccttggcggcccccggcgccacctgggggacgacctcctggaacagccgctccaggctggtggtggccgggtagtcgcggaaccacagcacgaactccgccacgcccgcggcgaccaggcgccgggcgcgctcggtgagctgttccggcgtcccgtacaggctgcgccgcgccagcaggtcgggatggtggctccagaacagcacctcgtgcggggtggagagccagcggtcgcgttccaggacggagtcgaagatccggcactccgcggcccaggcgtgccggacgccgtccggatcgagcccgagctccgtgcgacggcggcggaacgcggtgacggccgcggcgacctgagcgggctcaccggtccactggacatgagcgcactcccgcacggtggcgtcggcgggccgcagcgcgccgctcccggcgtccccggccggggtgcgcagcgcgagggggaggggctgctgccgtggggcgggcacgcagtgcgccgaagtgaggcggatgtgttccccggtgaaggtgacgggctgtccgccccacagcgcgcgcagggcctcgaccgtctcgccgagagcccggtggccggcggcctcctcctcgtccgcctccaggcccgtgggcacctcgcgccccgtcgagtggtgctccggcaggtactcgcgggccgggaagccgagggtgagccggccgtcgcagacgacgtccagggtcgcggcccgcttggcgatcagcgcggcgttgcggaacggcggggccgaggagagcagtcccagatccagaccgggcaccgcgcccgcgagggccgccagcgccgtccagccctcccagaccggctcgggctcgcgccggggcagggtgtcggtgcggtccagcagccagagggcgccacggccgtgccggtggacggtgcgggcggtctccaggaggagccgccagccctcgcggccgcccgtaccggccagttcgaggttggtgccctgcggggcgacgacgctccagcgcggggtcgcgggcgtcatgcgtggccgccgctgcggaccagcgcgagcaggctctgcggcttgtccagcaggaagtcgggccgctcggcgcgcagttggccggccgcgccctcgccccaggtcgcgcccacggtggcggtcccggcggcgcggccgctgcggatgtcgatcaccgcgtcgccgaccatgaccgcgtcctcgggcgccgcgtccagccggcgcagtgcctcccgcacgatgtcggggtgcggcttgggcctcggcacctcgtcgctgcccaccacctcgtccagcaggggcagcagcccgaccgcctccagcacggcgcgcgcccgggagccggacttgccggtggcgatcgcggtgccgacgccgtccgcgcgcagctccgccagcaactgcggcacgtccgggtacacctcgacgcggtccatcagccggtggctctcccggacgaacggttcctccatctcgccgggcaggcccatcagccgcatgatgtccgggaagtagcggccctggtgcgtgcggtactcctcgaagggcggctcgcccggccccaccacttcgcggtaggccacggcgaacgcctcgcgcatcaccgcgaaactgtcgatgaggacgccgtcgaggtcgaagaccacggtggtgaaccggcacgggagagcggtggcgggcgcgtccgccgcggcccgcgaggcgggcggggggttcaggggcatggcaggagctccgtgggtagggacgtcggggcccagaccccggcgtggctgacgactttcgtgaagcggcccgcgagccggtggtagacggcccccggcgagctgtgcggcgggtgggccgccgcgtccagcccgccccaggcggccatggccctcagcagcagcggatcggccggcacccagcggccgcccaccaggaactcggcccagcagtgcggtgtggagtacggcttggccaccagcagcccgaaggagaaccgcacgtcgagcccgcgccgccggccctccgccaccagccaggccgcggcgccgccgcagtcggccatgtgcgtgctccacaggaaccccgggtcccagcggatcgcttcgggcagcaggaagaagccgaccggctccagggtgcgcagcaggtcgaggacggccggggggaagtccggccagtggccgcgcgaccgtgtgtacagccgcgccagcgtggtctcccgcggcgaagccgcctccaccgggcggcgggcgccgggcagcagcactccgtagcggcacgggcccgcgtggccgggcaccgggcaggacgccgtgacgtcgacgcgccagcgcgggctctccgcggccgaggcggtccgcagggaaccggcccacgagcggatggcccggcgctgcaccgaggacaggccaaggtgcagggccgcgttgcccaggtcgtagccgtcgaagaggcgtcccgcgccgcttcccacgaagggcacgcccgcctcgcacagcgcggacagcagctccggtccgatccggtgcagccggcgcgcgctctgctcgtcgacggtgaaacggcggtgctcgtcgggcacgagcttcagacgctccacgagcgcctggagctcctgggtggaggggggccgggccggcaggtagacgctgatctcctcaatcctttcgccggggcgggcagccggtgcgggacggccggggcggccgccggtggggtgccgggtcagtcgatgtagcccagagcctggaggcgggccgccacctgctgctcgtcgctcgcggtgtacgccggctcctgcgggcgcggcgcgtagggggagaaggcgcgcagtgccggggccacctcgtgcgcgaacagttccatggactcgatgcggtgccggtccgccagctcgccgaaggtgaactcgcccagcagcgtgtcgaaatggccctccagggacgcggccctgatccggtcggccacggtcgccggcgagccgacgaagacgaggttgttctccagcaggaagcgcaggtcgccggagcccgccaggatgtccgtcgtgctgagcctgctgggtggggcgtgctgatcgcgggaacgggacaccgagccgtaccgcagcgtcttggtgaacgtctcggtgatgtgcgcggccgcccgctcctccgcctcggcgtccgtccgggccacgtggaccagcgtccagtagccgatgcgcgggtcgctcgcgtggccgtggccgcgccaccagtcgaggtagtgccggtagaccagcgccatggaggcgcgcggcacgatcatcgtggaactggtgtgggcaccggcctcgctgagatagcgcaaggtgttgcggttggtggtgggcacccacatcggcgggtgcgggcgctgcaccggcgggaaggacagggcgatgtcgcgcagccggtgcgcgggcccgtcgaagtcgaaggcgccccgcgcggcgaacgcggcacgcagcagctccagggcctcccgggtcaggaggtgacggttgtcgaaatcggcgtcgaagggcaggaaggggtcacggctgacgcccgaggcgagccccacctccagccgcccgcccaggagctggtccagggtcgcgacctcctcgaccaggtgcagcgggtggcgcagcggcggcacccaccccatcgggccgacccggatgcgctcggtgcgcagcgcggcgcccgtgcagaagaccgcgggcgagggcatccagctctcgtgcggcgtgcagtggtgctccaccgagaacgcgtagtcgaagccgagccggtcggcgtcctcgacctcgcgccacagctcttcgtagcgctcgcccggcgtgataccgggacgtccccagacatgggagaaataagcgaatttcattggcttccccgggtgggcaggacacgtggacaactggacgtgctgggcgccgcgtcgctctgcggccggtgcaggggcgagggagggtcgacgccggcggccgaatagatgtggtcgatgcaggaggccaggatggtcacttcggccatcgacccctcgcccaggcccgacaccgggccctgcccgtcggccgagccgccgagcctgcgggcgagttcgtccacctgccgccggtactccacgcccacgggctcgtcgggcagcgcgacggtctcctccaccccctggcgcaggaccacgaggctcgacttctgtagccggtgggggctgaagccgaaggtgccgcgcagcgtcgccaccccctcggtgccctccacggtgatcgtggtgacgtccagcgcctggtgggaggcccagcgtgtctccagggagatgcccacgtcggtgtcggtgacgaggaagccgcgggcggtgtcctccaccgtctccccggggcctggccgcgccgtgccggaggagcgccggctccagtcggccgtggcctcgccccggctcatccagtccgcggacatcgtgctcgccgcccggaccacgcgcggccaccccagcaggtgcaggcccacgtccagcaggtgccagcccaggtcgagcagcgcgccgcccccggcgagccggcggtcgacgaaccacccggtgcgctgcgggatgccggtggcccggatccagctcagcccgacactgcgcacggtgcccagcgagggcagcagctccgccaggcggcagacatcggtgcggtgccgggcggcgctccaggcgtagagggtgatgtccccgatgctgtcgccccgcgcctggtggtccagggcgagcgcctgggcctcgaagagcgtgcggcacaccggcttctcgacgaacaccggcacgtcccgctccaggagggccttggccacggggagatggaggtggttgggcagggcgatgatggccgcgtccacgcttctgggggcgagctcttccggtctgctcaggacgcgggtccgcgcgccctccggcagggccgacctggccgccacggggtcgtcgtccacgaggaagtcgacccggaacgccgggtgttccgccagcagcggcagccacaccttgcgcgagacccatcccgcgcccagcactgccatccggaggggttcgcgagctgtcgtcggcggtgtcacgacgggtgccttctccgtgaaagtcatcagaagcgggcaccaccgtcgacgacgagcgtattgccgttgacgtacgaggccgacggcgacagcaggaacgacaccgccgccgcgacctcctccggctcaccgagccgccccgccccgatgaactgcaggaccagatcgcgctcggcctccgagaagtccatcacctcggcgcggatcgccccgggcgcgacgacgttgacggtgatgccgtgcttgccgacctcacccgccaccgacgccgcccacggctggagcgcccccttggtggcggcgtacgcgctgtggccgggcagcccgctggtcccggcccgggagccgaagagcacgatccgcccgtacctggcgcgcatcatcggcttcaggcacgccttggcgagacccacggaaccggccaggttgacccgcagcagcttctccaggctccgggcgtccgtggccatcgcgagccggcgcgtacgcaagcccgcagcagccacacagccgtccacccgcccgaaccgttcgacggccgccgccaccagcgcgtcggcgccctcgggttcgctcaggtccgccgccacgggcacgagggtgccgccctggccctccacctgctcccgcagcttgcggatcgcctgctcgccgctgtggtagccggcgacgacggtggcgccgagcgcggcgagctccagcgcgcacgcgccgcctatctgcccggacgcgcccgagaccacgaccacgcggccgctctgtcccaggcgctccgtggcccggtcggtgacggtgctcatgaaccggcctccttggcgatgatcagatgacagggggacgcgtccagcggcacgtgccgtgcgacctccgccccctcttgcagcagcccggcgatcagctcgccggtggccggccacgcggtcccgccgtgcgtgagccagtccagagccagttcgctccccggcccggacgccggcaggaagacgtcgtcgaccagcagccgcccgcccggccgcagggagccgagcagggcaccgagagcgctgcccggccccgggccgtgcacggcgttggcgaccacgcagaagtcggcgtagccgacgggcagttccgtccccacggtcaccctgccctcctcgaccgccgcggcgacggccgaggagagcggcccgctcagccggccgacggtgaccagatggccgctcgccccggggtccgaggcgagcaggcgttccagatagcggcccgggcccgcggtcacctccaccacccgggctcccggcccgggccgcaggagccgcaggccgagcgcggcccgggcccgtgcgccggggccgtccatcgcgccctggtacagggcgacgagcggaccgaggctctcggggggacgctcctcgaagggacgccgcgccgtcccgctccgggccaccgcgacgagctcctcgcgcgtgaccagcccacgggagaggtgctcctccagcgccacgaacgcggccagctccccggcccggacccggtccccgggctcttgcgccccggtggtcagcacccccagagcggtggccgtgcgcagcagccactccagggcatccgcgtcgcaccccagctccccggcgaggagagccgtgccggcaccctgggcgagtgcctccagggcgcccaagtcgtgcagcgcgaagagcacttcggatgccttgtacgcccgggcggcctccgccgcgctccgggtgaggcggtagacggacgccgcccgcaccttgcccgcggcgttgaccggcaggctctcccgcaggacgaactcgtcgggcaccttgtgcggggccagctcccggcgggcgtgctcgcgcagcgcctcgggggtgagccccggccccgccgccgagacctccgcgacgatcccgtcctcgccccggtgccgcccccgccgggcgcccacccgcacattcaccacgtccggatgaccgcgcagcacctcctcgatctccagcggggagacccagcgcccgccccggcggatcgcccggtcctcgcgtcccaggatgcgcaggcccccgggcacggccacggcgagatcgcccatggcgtacggccggccgtcgacccgtacgctcagcagccccggggtgccggcgggcggcaccacgccctccgggccggtcagttcgcactccacccccggcaggggagcaccggcgcacaagggctccagccccgccggtccggcgagcacggcgcccgtctccgtggaaccgtagttgcgggcgagaccggtcccgaacgcctcggtgaacgcgcggtccagctgctcgtccaccggccccgcacccaccatggccagccggagaccgggagcggcgggcgcccgcccggccgctgctccccgcagccgccgggtcgccagcagccgggccacactgggcaccagggccaccacggtcgcaccaccggacagctccgcggcgatgcggccgagggcggtcggcggtacggggcgcagcgcggcacccgtcagcagtccgccgaacagccagcccagcgcgtacgcgtgggacagcggcaccggcagcagcagggtgtcctctcccgtcagcccgaccccgtcgcggtagcggcggccctccgcgagcaggctctcctcgctgcgggcgacgagcttgctcgcaccggtcgaccccgaggtcgggagcagcacggcgggcggggcgccggagggttctccgggcgagccggtcaacgtcaggcggaggccgtcgccggtcccggggacgaccagggacctgccgcccccggcagcccgcagcagccgcgcggtctcggggccgggggtgtcgggttcgagcagcaggggcctggcgccggatgccagcagggagaggaaggcgacgacccaccgcgggctgttgggcgcgcgcagggccaccgcctcgccctccaccgcctcggccttgagctgtgccgcggccgtacgcacctgctccagcagggagttcacatcggtcccgggcagcggtatccggccggacggcagccgttccaccgcgcccaggagggtgtccgcctcgtgaccggtcgcctgttcagtcatggccgccctggaggtagtcggccttcgcgtcgctgagcatctgccggatgcggggcggggagtaggtgggagcggcgcacagctcgtcgagggtgatctcgtcctcgaagtacatcgagagcgtgtcccagggcgtgaagcagccgtggcacgccttgagccggggcttgggggcgagcagggcacggtagaggccgctggtgcccaccgtgtccagcatctcgctccagtcgtcctccagggcgttgcccatgtcggagaaccagatgttggggcagggcgtgacgacgccgtcgctgaagctggagacgaccagccgcggcagatggcagcggaaggtgcggcggccctcgcggtagaagctcgtcagccggtcgaagtagggccgcggcgggaggacccgcgcgaactcgtcgtagcggtcgacgagttcctggatgtggccgaactgcccgggccgcaccttgaagtcctccgagtccgggccccgcaccgggaaggggaagtagacgggaggccgggagaatcccgacagccactcggcgaacgcgcagacctccgtgacgctccggtcgttgagcactgaatagatctccaccggcagccccgagtccaggatccgggcgatggcggcgacgaccttctcgtgcaggctcccggacggcacacgatggctgttgccgtggtggaggtggctgtcgagggagacctggagcacgacgttgccccacgagcggaaccgctccaggtgctcctcgcgcaccaggacaccgttggtctggatgaccagcacgtcgtatttacgggcctcctgctccaggaagtccatgatcccccggaccaggaagatctcgcctccggtcaccttgagcagcggcaggccgaagcggtcccggatccggtcggcgaccttgtccatgcgctgccccagcccgctgtccttggcgtagctgtcgcgccgcgggggctcgaagatcagttgaagggagtggccctccttgaggttgctctgtccggtgaggcagtaggtgcagctgaggttgcaggagtcctcgttgatgaccaggtcgttgccgatcagcggcaggcgtcgcctggtgcccgcgtcgggggtggctgtcggcgggtgggtgctacgggacatgagtggcctctctcgtggtggggctgcgcacggcgtgggtcacggccccctttccacgggtgccggccgggcctcgccccggtcgtcctggccgacgggcacccagtggcggtcggcgtggccgggccggccgggcccgccgtgtgcgcgggcgcgggcgaacagggcctcgcgcagggcggccgcaccgccgaagtgggcgctcgcctggctggagtagtgaccggcggcggtcagccagcggtccaccgcctcggacggcagccgttccgtgcgcggccgcaggttcgtgtggtcacgttcgcgcagccggtaggggaagtcctcgtagaagacggtacgggcgggggacaggggctccaccgcgccgcggaccaggcggtggtcgacgtgccggcccgccgccaggggaacgtggacgctcgacgcccccgcgcacagcggcagcagagccgcccgcacctcctccagcagcgggaggtcggccgggtgccaggggccgaagagccggcgcggggaagcgtagagataggcgcccgaggccgtacgcagtgccgcgtcggtgaagcccagcggcacgtggcgcacgcccagttcggcacatgccgcccggtcctcggcccggcgcaccgcgggatcggcggcgctccgccacgactcgggcttcccggccgcgggcccggcgaagaccgtgacgacggtcggccgcgggccctcggcggcccagcgcgccagccgcccgcccagggaccacacggcgtcgtccgcgtgcggggagagcaccacgggaccgtacggcgcggtggccggtgtgccgctcatcgcgccgcgtcccgtgccggtgcggcctcttcggccaggacggcgcgcacatacgcctccggcgtgccgtcgggcccgagcagggcctccaaggcgcgtaccgacgggtgcgggtggccggcggagaagtgcgtgaggttgcggtggtgctgctcccagcggccggggcgggcgtggctgaggtgcacaccgagggcgtcgggggcgagggtcctgcgcagcccggcggcgtgcaggcggaagccgaactccaggtcctcgcacccccaggtgagcccgaactcctcgtcgaatccgccggtatgctcccatgcggccttgtcgagggcggtgttcgcgccgatgaagccgagccagggggcgacgtccggcagggagccgccggccatggcctccacggcccgctccagggcgttggcgacgagccgccggtgcggttggcgccgctcggaggcggccggggcagcgggttcgagtccggcgcgggcgcggcggacctcggtcggggcggccttctcgacagcggcgaggaaccgcgccgcggtggggagttcgcgcagccggccgtgggtgaaggcgtccggttccgcggccgcggcgtgtgcggcgaggaagccgggccccaccaggacgtcgtcgtcgaggaagaccagccggggcgcgagggccgcggccgccccggcgttccgggcggcggcccgcccccgcagcggtccccgcaccacgcgcagcgggagaaggccgctcatctcgcctgtcacggcgatcagttgatcaccggcgtcgcccccgtcgttgtcgtcgacgacgaccacttcgaagggcggcgttcccggggaggggccggcaaggcatgcgagggtcgcgcgcaggcgtgccgcgggcccccggctggggacgacgacgctcagccggggggcggtggtgccgttgggggcccgcatcgggtcagagcgcgccgacgaggccggagaagacctccgccagccggtccagcgtggtcacgtcggcgagcaggacgcgatggtgcagccagaggcagtcgctgccgatctcctccgccacgggacagctcttggccagctcctcggcgtccgccggcgccgggccgcgcgcgaaaccctcggtgcggtagaccggcgggaagccgacgaacgcgggcactccccgctcgaccagcgcgtccaccagcgcgaggcggcgccgggccgagatgccgggcagccggaccatggccatgtagtgggagtggaggtcgccgcgctcgtcgcgcccctgcggcaccacgccgtcgatggcggccagtgccgtacgcagctgggcccagcgctcctccctgatgcgcaactgatccttcaagcgcttcagttgagcacgcaggacgctcgcggagaactcgttcatgcggtagttggagccctgcgtcagatggcggtagacgcggtccccgggcgggcggccgcagcagtgctggaggaacgcctcgtggaaggactcgtcgtccggcaggagcagggcgccgccctcgccggcggtcatcagcttgccgttctggaagctgaaggcggcgatcgagccgagctccccgacccggcggccctgccactgcgcgccgtgggcgtgcgcggcgtcctggaggaccggcacgcccgtcgcgacggagagcttctccagggcgtccatgtcggcgaactggcccgccatgtggaccggcatgatcgctttggtgcgtggcgtcaccagcgccgccgccgcgtcggcatcgaggcagtaggtgtcgggccgtacgtccgccggcaccggcaccgcgcccatgcgctgcacggccagcgacgacgagatgaaggtgaacgcggggacgatgacctcgtcaccggggccgatccccatgacccccagggcgagttccagggcgtgggtgccgttcgtcgtggcgatcgcgtgcggggcgccgtggtggtcggcgaactcccgctcgaagagatcgacctcctgccccgcgtcgcgccaccaccccttctggtccagggcccgcaggagtccggcgcgctcctccgcgccgtgttgcggccatgagggaaaggacaggacgtcatcaccggacgtaggtgtcattgagcagcctttcggtcctgcgggtgcggcggcacggtcgacttcggggcgctgtacggcgggagggcgggtgtcgaggcctttgccttcggtggccgtggcttcacggcccggttccgttgtgttcgccccgcgtcgggaagggtggtgcgtgacccgacgggaaacgccgttcctcgggggcggcgggaaatccggcccgcggtgtgaggggtggccggagcgggcatgtgatccggcccccggtcataggccgggatcggatgccagacacgagcctccatagggcagttgccggagtcaacacccttgccgggaaggtctgccccgaccgccggtcggcggtggattctcgtcagcagggtggttgagcagtgaaactgccttattcccaagggaattgatccagttcaggggctgctcggcgggcctgtcggcaacgttatccggcgtcgaagtggctcaaaccgcacggctggagggagcgggaagcgtcgcgtatggtgggcgcgacaccgtcctggtatgtgctgtgtgcatggttcattgagccgaatcccactccggccctcggatccgggcgccatacgatcaccgttgtccggtctgtggacgcaccggtgaggggctgttacagtcctcggatcatcgatgagcggcggcagtttctgcctgcaatcgtgatgagttctcagagctggaggcaatttcgtgccaccctctccccgcgccctcgtcatcggaatcgacggaggcacattcgatacggtcgacccgctgatcgagtgcggtctgctgccccatatggcgaagttgctgcgcgagagcgccagtgccgccacggactgcacctggcccgcccacacggcgccggggtggagcacgttcgtctccgccagcgatcccggcggtcacgggatctatcagttctacgacacccaggacccggcctacggggcccgcgtcacgcgctccggcgacctgggccggtcctgcgcctgggactggctcgccgcgcaggaatattcgctgggcctcatcaacatcccgatgtcgcacccgccggccgacctccccggctatcaggtcacctggccgctggagcggacactcaagcactgccgcccggattccctgctgcgcgaactcgccgcggccaaggcccatttccagtcggacctcgcgaccatgttccggggcgacatggcctatctggaggaggccgagcgcaatgtggcggcgcgggtccgctccgtacggcatctgatgagcacccggcccaccgatgtcgtgatggtcgtgctcaccgaggccgaccgggtcggccaccactactggcactacggcgaccccggtcacccgggccaccggcccgccccggagggcagcggctgggacgtcgccatgccccggatctaccaggccatcgaccacgcggtgggcgagctcctggagctcgtggacgaggacacctccgtcgtgctcgtctccgaccacggcctgggcaccgggcgccacggcctgtcggtgcacaccctcctggaggaggccgggctgctggccaccgcaccgggggaggagccgcaggacgcggcggcgagctggttcgcgggcaacggccggcacgtcgacttccgccgcaccagcgtctacatgcccgtccccggcagctacggcctcaacatcaacgtacgcggacgccagcagcgcggcaccgtcgcaccccgcgaccgcgaacgcgtcatggacgaggtcacgggcctgctctccgggctgaccggccccgagggacagcaggtcttccgggccgtccgcccgcgcgaagaggcgtacccagggccgcacaccggccgggcacccgacctcctcctcgtcccgcgggacgagaccgtcctgcccgtccccgacctcggcggtgacgtgtggcggccgagcgcgcagaccggcctgcaccgctaccgcggcctgtgggcgcaccgctcgccccgcgtccgccccggccgcctgcccggcaccgtcgcgctcaccgacaccctgcccaccctgctcaccgacctcggggccgcatggcccagcgacatccacggccgccccgtgaccgccgtcctcgacgacggcgtacgcgtcccgccctccgacccccgggtcgaggccaccggcaccccggccaccacgatcccggccgccgcttcggccgctgatgccgccgaggacgcgtacaccagcgaccgcttgcgcgaaatgggctacctgtaagcaccgccgggccgtaccggcgcttgtccccaccggagtcccgccgctcgcggcggcgtggaggagagaggtatttctgccatggagaccctgacgaccgacaagatcaaggaccggctgcgcaaggtgctcgtcgattccctcgaactgtccctggacccctcggccgtacccgacgagggactcgtggagaagctgggcctggactcgatcaacaccatcgaattcctcatctgggtcgagagcgaattcggcatagagatcgccgacgaggacctgtcgatcaagctcatcgacagtctcgacctcctcgccggctatgtgtccgagcgcgtgaacggcgtcaccgcacccgccgaatgacggccgtgcgcgcgctcgcctccgggcccactccccgcagcggaaggacgtgagcacgatggaccggcacgccctggtgatcgggctcgacggcatgccgaggaccctgctgacccgcctggccggcgacgggaccatgccgcacaccgcggcgctgctcgccgagggccactgcgcggaactgctggcacccgtaccggagatcagctccacctcctgggccaccttcctcaccggcaccaacccgggccggcacggcatctacggcttcaccgacctcgcccccggcgacggctaccgcatcaccttccccggtgtgcggcagctgcgcgaacccccgctgtgggaactcgccgcccgcgccggccgcaggaccgtgtgcctgaacgtgccgggcacctaccccgcccccgccatcgacggcgtgctggtctccggcttcgtcgcgcccgaactggagcgcgccgtcagcccgccacggctgctgccgctgctgcgcggcctcgactacgaactcgacgtcgaggtcggcgacgtcgccgccgacccggccgccttcctcgggcgggccgtccgggccctgcgcgcccgcacccgggcgatggaacacctgctgcgccaggagacctgggacctcgcggtcgccgtgctcaccgagaccgaccgcgtccaccacttcctgtggcgcgcggtcgccgaccccgccgaccccctccacggggacgtcctcgccttctaccgcctcgtggacgactgcgtcgccaccctggtgagcaccctcccaccgggcggcgaactcttcctgatgagcgaccacggcttcggacccgccgcctgtcaggtctatctgaacgcgtggctcagggagtccggctggctggccgggctcgacgtctgtccggacctcaccgcggtcgacgctcgcagcaccgccttcgcgctcgaccccgcccgcatccacctcaaccgcaagagccgcttccccggcggcggcctgaccgacgcggaggcggacgaggccgcccacgagatcgcgcgcgagctgtccgccctgcgctgcgacggcacccgcctgggccccgacgtcgacggacccctgctcgtccgcgacctctaccgcgctcaggagatctaccacggcccgctgttgggcaacgcccccgacctggtggccgtaccggcccccggggtgcagctgcgcggcggctggggcggcacgcacaccgtacgcaacgacatcctcaccggcacccacacccgcgacgacgcggtcttctaccggcgcggcgcgcccgcgcccgcccccggggcggacgacggccccctcgacatgacggacgtcgccccgaccgtcctcgcctccctgggcatccaccccggcgggctcgacggcgcggccgtactcggcaccacgggacccgcgtccggtcacggccgcacggacccccctctcgacatcagggagctctgatgaagcacgacctcggtctggcaccatcggcacccaaaccgggaacactcgacctgagcctggacccacgcatcacggaccccgcttccttccgggtcagtttcctgatcctcctcgacggcgacctcgtgatgtcccccgaacacctcggcgtcgcctacatggccggtgtgctgcgccatacgggcttcaccgcggagatccgggaggtggagcacggcgacgaccaggcggccgccaccgtcgaggcgctcaaggagtaccggcccgacctcgtctgcttcaccctgatgagcctgaacctgggcagctgtctgaccctgtgccggatgctgcgggaggagctgccggggacgacgatcgcctgcggcggcccagccgggaccttcgcgggcctggacgtcctgcggaacaacccctggaccgacgtcgtcgccgtgggggagggcgagcccaccatcctcgacctcgtccaacggctctacctcaaggagccgttgtccgcctgcaaggggatctgctaccgcgacgaggacggcacaccgcgccagaaccccgcccgccccctgatccacaacctggaggacctccccttccccgcccgggaccagctgcgccagcacggcgacaagctggagtacgtccgggtcagcaccagccggggctgcgtcgccaactgcgccttctgctccgccccgcacctgaagaaccgcgtccaggcgggcaaggcgtggcgcggccgcgggccggaacagatcgtggacgaggtcgccgagatcgtcgaacgccaccagttccggaccttcgacttcgtcgactccaccttcgaggaccccgacggcggccgggtcggcaagaaacgggtcgccgccatcgcgaacggcatcctggagcgcggcctcgacatctactacaacgtctgcatgcgggccgagaactggcacgacacccccgaggaccacgccctgctcgacctgctggtcgcctcgggcctggagaaggtcaacgtcggcatcgaggccggcaccgccgaggaactgctcctctgggagaagcgcgccaccgtcgaggacaacgtcaccatcatcaggatgctgcgggaacacggcatctatctcgccatgggattcattcccttccacccctacgcgaccctggagaccatcgtcaccaacgcggccttcctgcgcgacaattccggccacaacctccggcgcatgaccgaacgcctggagatctaccccggaacggccatcgtcagccgcatgcgggccgacggactcctcggcgagagctatctcgaagggctcgacccctacggctacgcattcaaggatccccgcgtcggacggctcgccaagcatttcgcccagctctacaacaacgacgactaccaccggcacggcgtcatcaccgagcagtcctccgtcttcgccttcgagacctacaacgtcgtactccagaccttcatctcccggctgcaccgccggttcaccaccctgccgggggtggacgaggtgatggaggcattcaaggcccgggtgcacgagatccgccaggagatgggccggcacaactacggcttcttcatgtccaatgtcgaggcggtcatgaacgacaccctcgacccggagaagcagcgccggcaggtggtggacgtcgagcacttcttccgcgaccgcctcgatgtgttgcgcagcgagcaattgcgcgtcggcaaggccctcacccggctcggcgcccgggtgacggaggtcagctcgaccattcccaaggagcgccccggcggactgccgcgccagtacacgggagagggcagcggtgccacgtggtgagacgggaaccgccgcggcgcgggtggcggtctgcacgctgagcagcagggaactggtcggcccgctggcccggttgcccggtgtggcggccgcgggcacgctgatgaccgccaacctgggcatcgagcaggtgatcaaggccctgcggtgcgaccggacggtccgcggcctgctcgtgtgcggccgcgactcaccccgcttccgcgccggccagagcctgatcgccctcttccgccacggcctgcgccccgaggacgggcacatccggggagccaccggctatctccccgtcctgaggtcggtgacggcgcgggagaccgaggaggtacgcgcccgcgtcgagctggtggacgcccgtggcgagcgcgacgtcgagacgctgcgcgccgaggtcgcggcactcctcgcccgcgtacggcgcaccccggccctcccctcccgcgagcacgacggcggccaacccagcttcgtggagccggacttcggacggctgcatcctgtcggccgccgccgctccctggacgcgggcatcggcgggttcgtgctcatcagcgtcgaccgtgagcaccggcggatcctgctgcgccactacacctccgatgtgcggccccggcacgagatgtggggcacccgcggggaggcgatgctgctcgggctgctggaggccggcgtcatcgaggaccccgcccacgccggatacctcggcgccgaactggccaaggccgagacggcgctgcggctcggcctgcactacgaacaggacctgcccctgcgcccgccgggcaggccgcccggccctgtgcggcgccggaccgcgaaggagcgaacgaccatggcgcaagcacccgcgctggaggacttcctgcgtctcgtgacgaggacgctgggggccgaggacgccgtcctggacctgcacacgccgctcggcgagcaactggcggtggactccgcccggctcatcgaactcaccgtcgtcctggaggaggagctcggcgcggacctccccgacgacgccgacctcgccagggccacccccgcggaactccacaaagcactcgtgggctgaggaggagaccgacatgcgcagcgtgctgttgctcaacggacccaacctggggacgctcggcaagcggcaaccggagatctacggaaccgacaccctggccgagatcgaggccgccgtggccgaggaggtgggagcgcgcggctgggaggtggtctccgaacagcgcaacggcgagggggaactggtcgatgtgctccagcgccacgacgacgtggtgggcgccgtggtcaaccccggcgccctgatgatcgccggctggtcactgcgcgacgcgctcgccgacttcgccccgccctgggtggaggtgcacctgagcaacgtgtggggacgcgaggcattccggcacacctccgtcacggccccgctggcctccggcgtcgtgatggggatgggggcgctgggctaccggctggcagcgcgcgccctcacccggctggtccccgaggactgacggtgacccggcccggcccgtacgcacctccagatgggaccggcccgcccggcagggacgccacctcggcgcccggcccgtacgcacgctcaggcgggccacacccgcagctcctccttgatcacctgagcgccggcctggtcgcacgccccgggcagcgggcaggccgccgggaggatccgcacggtgaacggcccctcggtcaggccgcgccaggcggggaccacggcgcgaccgcggtccacctccgcggacaccaaggccgtgaccggacagggaaattgaccggagacctccccgcccaccccttcgccgggcccgcggctgccgagccacagcagcacatgcaccggcgggcgcgcggcccgctcggcggcccgcagcgccgcgcccagcccggcaccggcacagaccagcgcccacggcccgccgtccccggccgcctccgcgatcgcctcgacgccgtgctccaccaccagttcgggggcgagcgcctgccgccagccctccgcgtccggcccgtccgtcacggcgaccagccggaccaccggcaccaccacgcctccggcgttcccctcagccgtacgcgacatccccagaccctctcttccgtaccgtcccacccgccctcgctctcccgcccggcgccgctacggcaggccgtcggtcatcccgagggagaagtagttctcgtaccccagcagccggcgcagttcgggcgtcgcgcgggtggcgacgtcctcccgcagacccgagaagaagttctgctgctgccggacgtagccgcggcagtagtagttgaggatgccgtgccgcggccggtcggtggtgttggcgcccgtctggtgccacaggcgcccgtcgaagaccatcacgctcccggccggcgcgcacacggcgaccgtctcggtgttcccctcgccccggtcgtagtccggctgccggcccagcagatgggagccgggcaccaggcgggtcgcgccgttgtcctcggtgaagtcgtccagcatccacatgctgttggccaccagcggatacgggggccacggcgggcgggcgaaggtctggtccgcgtgcagatgcatccgggaaccgccggggcccgcgatattggcgtgcgtgctggagagcaggaagccgaagcccaggatctcctccatcaggagcatgacggtgggatcctgcacgttctgctcgaattcctcgcccttgttcagcaggctgaagacgcgttggttgccgccgtcgtagagaaaggccgagccgttctcacgctcctgctcggcgacctccagcagccgccctctgagcttttcgaagaccgcggccggcaaggggcactcgatcaggcagtatccggcttcgaccagatcccgggaggctttctcgacgtcattcgtcaaagtcgcatccatatggcgaggctagcagccgaaatctcggccgcaccatagcgcgaaaacgccggtccatgattttttcacgtgcgggaaggacggattttccatggcacactcaccgcggcggccggacggccccctccgcatcggggtctggctggccccccagcacacctcggtggccgaactgcgcgccgcctggcgcgcggccgactccctgggcgtggactcgctgtggctgtgggaccacttcttcccgctcaccggggaccccgacggcagccacttcgaggcctggaccctgctggcggccatggccgccgacacccgcgccgcccgcctgggcaccctggtgtccaactacgcctaccgcaaccccgacctcctggccgacatggcccgcacggtcgaccacatcggcgacggccgcctgatcctcggcatgggcgccggctgggtcgaacgcgacctgaaggagtacggctaccccacgcccggcgcgggggagcgggtggacgggctcatcgaggcggtggagcgcgtcgaccgcagactcggccggctgcgccccgggccgctcggcgacctccccctgctcatcggcggggacgggcagcggcgcctgctgcgcttcgccgccgaacgggccgccatctggaacaccatggcctggcgcttcgccgagggcaatcgcgtgctggacgagtggtgcgcgcgggtcggccgcgacccggcggagatcgagcgcagcgccttcgtcacccgcgaccagaccgacgaggagctgcgctgcctggtggcgacgggcgtccagcacctgatcttccaggtcgggcaccccttccgcttcgacggcgtggagcgggccctgcgcttcgcgggcggctggagcaaggggtaaggccagggcccggacgcgccccgcgtcgccactagagcaacgcgtccgccagccggtccacttgggacagcgccgccgccgtggggtggaggacgacctcgtccaccccgccgtcggcgagcgccgagaccgccgcgcggagctgccccgcggtgcgcggggtcttcgccacgaactcctccgcctcctcgcccagcaccgcgaagtagtcccggacgaaggccgccgactcctgggccacgtcctcgcccagggtgtagcgcgccagcgccaccacatgcggcgccccggcgcgtcccgcctcgctccaggcgcggcgcacccgttccgcgaccggcacgatccgctccggctccaggccgggcgccgtccagccgtcggcccagcgcgccacgcggcgcacggccgccgcgctgaccccgccgacgaggaccggcacaccggggccctccgcgcccggccgggcgccccggccgagcagctccagctgctcctcgaacgccgcgcgccggtcgtcgaaggcgcggccggcggcctcgaagtcgtcctcgcgcacgccgggcccgacccccagggtgaaccgcccgcccgacagcgagtccagactcgcgaccgccttcgccagcacaggcgcggtgcgcagcgggccgatcaggacattggtgagcagcccgatccgggaggtcgccccggccgccgccgccagcgccagcagcggatcgtggcccggatacaccaggcgctcggtggccgcgagcgaggcgaatccccgctcctcggcccgccgcgcccaatcggttatcaggcgcccgtccgcgccgggcacggtgttcggcagagcaatgctgatcttcattggtctccccgggggttcgcaggatttccggtcgaatgtgacaggggattccggcacggccggcgtgattgcggcaggagttcaccagcggcccggcgcggagaaatgcggcggcatttccacggccccctgtcggaccgccggaccgccgtgtacgtttttcggaaagcaacgtcgtacggtgcgcacagcgagaggaatccgcgatgcccgctgccggaaaagtcgccgtgataggactcgactccgcgactccgcagtacatgttcgaccggttcgccgaggacatgccggtgttcaccgccctcaggcgcaagtccctgtggggtccgatgcgcagcatcgacccgcccatcaccatgcccgcctggtcctgcatgatgtccggccgctcgcccggcgaactcggcgtctacggattccgcgaccgcggcgcctacgactacgggccgttgaagttcgccacctcccacagcatccaagccccccggatctgggacgagatgacggccgccgggcgctccagcgtggtcctgggcgtccccggcacctatcctcccgcccccatccgcggggccatggtctcctgcttcctggctccctccacacagtcgcgctacacctccccgcccggcctcgccgacgagctggagaagctcaccggcggctacgccctggacgtggaggacttccgctccaccgacctggaacgcgtatcccagcgcgtcttcgacatgagcgagcagcgcttcgaggtcgcgcgccacctggcgaccacccaggagtgggacttcctctccttcgtggacatgggccccgaccgcctccaccacggcttctggaaatactgcgaccccgaccacccgcgccacgagccgggcaacgcctacgccggtctcttccgcgactactaccgcgccctcgaccggcacctcggccgcttcctggagagcctgcccgagaacacgaccgtcctggtcgtctccgaccacggcgcccagccgatggtgggcgggctcttcgtcaacgagtggctgcgcaaggagggttacctcgtcctgaccgaggagcccgccggacccacccccgtcgcccaggccgccgtcgactggaagcggaccaccgcctgggccgaaggcggctactacggacggatcttcctcaacgtcgagggccgggagccgcagggcaccatcccggccgcggagtacgagagcacccgcgacctcatcgcctccgccctggaagcgctgcccgacgaccaggggcagccgatgggcacccgcgccctgcgccccggcgagctctacggagaggtcaacggcatcgcccccgacctcctggtctacgtcggcaacctgcgctggcgggccctggccaccctcggcatgggcaagggcctctacacgacggagaacgacaccggccctgaccacgccaaccacggggacaccggcatcttcgccctcagcgcccccggcatcacccccggccgcgcggacggcctgtcgctgtacgacgtggcccccaccctgcgggaactgctgggtctcgcgccgcagggctcccgcggctccctcctcggctgacatcacccgcccagcagcgcgtagggagtgggcggcgccggcaccccgcccgctcccgcaccgccaccgtgcaccacgtgcttgtggcggtaggcgtccagctcgttggtgagccggtcccagacggcggagcggggcccggctgtgccgggcagctccaggtcgaccagccggtagtcgttgatccatacccggtccgcgcgcagccgctcggccaccgcgcgggcccgcgcgggatcggcggaccacaccccggcgctgagccggtagcgggagccgttggcgatgcgcaccgcgtcgtcgtcggacccggcccggacgaccgcgagcaccgggccgaagatctcctcctgcgcgacggcgtcgtccgcgccgaccgacgccagcaccgtgggcaggaaatacgccccggcgtccagcccgggcggcagctcgtccgccgcgggcgcccggccgccgcacacgagctccgcgccctgggagagcccgagttcggtgaagcgcctggccgtacgcgcctggttgcgcgagaccagcggccccaggtcggtggccgggtccagcggatcaccgacgcgcagccggcccacccgttcgctcagcagccgcaggaagtcgtcgtggacgtcggcgtgcaccaccgcgcgggtaccggccatgcacacctgcccgttgtgcaggaacgctccccacgtgacgccggtgaccgcccggtccagatcggcgtccgcgagcacgatgttgggggacttgccccccaggtccagccgggcgctcgtccccgccgcggcggcaccctcccgtacggcggccccggtctcgtccgagccggtgaacgccaccaggtcgacgccgggcgagcgcaccagctgctccccggcgaccccgcccggccccgtgaccacgttgaccacgcccggcggcaggccgcactcgtggagcagctccaccagtcgcagcgtggagagcgaggcgaacgaagccggtttgatcacacaggtgttgcccgcggcgatggcgggcgcgatgcgccaggccgccagcagcagcggcagattccacggcacgatcgcggcgacgacccccaccggccgccacacgacgtaggaacccgaaccgggcgcctccggctgccgttcgggcacgtgctccgcccaccacgcgctccactcgaaggctgccgcggcccccggcacatcggccccgagagccttgcgcagcgtcgagccgttgtcgcgggcctccaactcggccagcggctccgcttcttcacgcaagcgctgtgcggccttgcgcagcaggcccgcccgctcgcccggcgccatccgcggccacgggccctcgtcgaaggcccgccgggcggcggacaccgcccggcggacgtcctccgcgccgccgctgggaaggtcggccaggtggcgccgcgtggccggctcgaaggtgcgcaggacggcgccgtcgtgggcctgcacggcctgcccgtcgatgtacatcgggaaccgctcgaccgctctgtccacccggtccatcgccttcaccttctccttctgctgacccgtggggatgcgcccggccgggcccgcccgcggccgcggccgtaccggaacacccgccccggagcggccgcgcccgcggtcaggccggcaggggcgggatgttggggttgaaccggaagacgttgcccgggtcgtactgcgacttcagggcctggagccgcgcgtagtcctccggcgtgtaggcgctgcgggtcgtctccctcgatgtgttgtgacccgcgaggaagttcaggcacaccccgggcgtcgtccacggccgcatgctgtcgacgaactcctgctgcgccgcgtccacggccgccagggtgtccgggtcgaccagcgagcccacgtaggcgttgaacaccgcctccgggaagtggcccaccgcgctcgggtgccggggcggccgggcgagggcgccgcccaggtgccgcagctccaccccgaacagcgcgtccgtgcccggccccgcgagcctgaggatctcgtcgacggcgatctcgtccagctgcccgaacatgaccgttttgctgtgactcgacaccggggccggcggatcgttgtggatgatcccggcccgcgtgtacgggagcgtgtccaccgtatccatgacgaccgtgccggcggcccggagctcggcgaaccggcgctcaccctcctcggggtctcccagccaggccagccggatgtgggtgacgaaccggccgcgcagcggtccgggcaccccctcggcatcgggatacgcggccaggaacaccgacgacgccatgtcctcgggcatccggggcgcccactggagataggtgttcagcacggcgcgcgtggagccggcgtcgaagaacagccctccgccgtacacctgggtgacggggaacagcccgacctcgacggaggtgacgatgccgaggttccccctgctgccgcgcacgccccagaagagatcggggtgttcctcggcggagacctggagaaaccgcccgtcggccgtcaccaggtcgagcgagacgacatggtcgccggcgaacccgtacttccgcgacagaagcccgagcccgccgccgaggaggtaggagaccgcgccgacgaacggcgccgagccgctcagcggtgcaagaccgtgcgccgccgcctcgtggatcacctgctcccagcgcacgcccgcctcgatccgggcggtccgggcccgcgggtcgaccctgacgccggtcatccgccgggtgctgatgaggacgtcggtggccgccgaggacttcccgtgaccggtggcctggacggcgatcccaaggccccgggccctggcgaagcgcacggcggcgatgacatccgcggcaccggtggcgacgacgacgagggcggggcggtgttccacggacagttcgaagccggagcgctcctcgtcgtacccctcgtccccgggcaggaggacggatccggcgacctgcgccgcgagctcttcggcggccgcggcgccgagggccgcggacgtgtccgtcacggagtggctggctggtttcaccgaggaacctttctggctggagcttcgagaagcgcgccgcgcgtgcgcgggcagggccgcggggctcgccggcccttggaacggagcgggccccgtcagttgcgcgggccggggaccaccggcagtgaccggacgcccaggggcaggagttcggagctgtgctcgacctcttcgggaggcacggccagggcgatgcgggggaaggccgccagcaggcggccgatgccgacggtcagttcggtcacggccagcccggtcgcggggcaggcgtgctgcccggcgccgaacgcgatgccgcggtcggccacccgctcgatgtcgagcacgtggggatcggcgaagacctcggggtcgcgggaggccgccgagaccagtggcagcacggcgtccccggcggcgacgcgcctgccggagagcaccacgtcctcggtggcgacccgcagcaggccgtcgttgctggaggggtagtagcgcagcaactcctgtacggcagagggcagtacggaggggtcctcgcgcagccgggccgcgaggccgggggaggagagcacgccgaagaggtgccgggccagcaggtcgcggatggtgatgaagcccgagatgatcaggccgtggagcagcaggcgccggtcgtcgtcggtgagctcctccgcgtccagcagcgtgtcggtgacgctgtcgcccggctcggccctgcgggccgcgagcagttcgtccagcacctggccgagcctgccgcgggcctccttcagcgcctgctcggtggcaccgcgcggaagcagcagcagctcgacgtcggaggtgacgtcctgccaccggtccccgggcagcccgaggaactcggctgtgacgcggccggcgaagggcgcggtgtacgccgcgacgaggtccaccgtgccgctgccggcgggcagccggtccagggccgcctcggcggccgcctcgatgcggggtgcgaaccgcgccgtgcgcgaggcaccgaacgcggccaccaccggaccgcgcagccggccgtgctccggcgggtcgaggtccacgatgcccgagccctgggaccggccgaagcccgagcccggcagcatggcggcgcgatggcggctgaagcggccgtcggtgagcaccgtgcgcacgtcctcgtgccgggtcaccagccagatgcgcgagccgtccgccaggcgcacctcggcgaccgggtcatcggtgagcagccgcgcgtactcgggcggcaccgtgccgccggggccgggcgggaagggaaaggcgggcggggcggctgaggtcatgcgccccggctcctctcaccggccggcgcgccggcgcgggcgtggcccggccaggtgaagtccttcgccaggacgcggttgtccagctggtgttccacgacgatgttgccgcagccgtagtcgtcggcgatgacctcgacgtcctcggcggacaccaggtgtccggggtcggccagcagggtcgccacgcccttggcgggggccaggacctccagggcgaagccgcgggccacgtgctggcggtgggtgtgcgccgccgcgatcaggaaggtgtcgtcggggtcgcagatcagatgccacagcgcgcgggcctggaggaagcggcgcgggcggtcggtgaagcgccgggaggagaccaggaccggcccgtccgcgcccctgcgggtgccgagggcgaccaggccccggtccatataggggacctgctcggtgcggtagcccatcaggggggccgggtcgaagggcgcggtgtcgtccagacccgcccacgcgcgcacccggtgcgccacctcgtagccgaggtcccaggggttgtcgagtcgccccggagcgaagaacttctcgctcaagtcggcgcagtccttgcgcagttccaccaaggcgggagggagcggggtatccgcgggcgcggtgcccatcagggtgcggacgcgcgcgatccactggagctggtcggctatccgctcaggccgcaccccgttgaagaagtcactggcgagcggttccgccaactgctcggcggccttgaggatgtccgcctcgtacggctcggcctcggcgtaggggtccaggcccagccgtgccgcgatgcggcagaaggcggcctcgtcctcgtcggtggcgcggacggcggcccactcctcctggagcggggtgccggtgatgccctggtccgtgaggcgctcggtcaccgcgtcgacgaacgaggccagtgtggcggtgaacgcggcgctctccacacaggagttgccccggctcgcgaagcggttcccgggtcgtacgtcggggcccatgtccggcatccatacgatccgggtctcccggccctcgggcacgaagagcatgtccggccagcggaagccgtcgcaggcggcgcgcaggatgtgacggcgcgaacgcatccaccacgaaccgcggttgtcgcccacaccgtggcggtaggcgaagcgcagctgggatatctgggtgccgggccgcgcgtcggccaccagcgaccaccagttgaaggcgatccactcggccagggggtagagcgagccggtcgtgtgctcccggaaggtcccctgcccgggctcctggacgagtgtgacggtctcggcgcccacggcgatgcgcagccgggcccaggtcgcttgcagctcgcctccgccgccggccggggcgtcgagccaattccactgcaattggaactcaggaagcatggtccgccagcccttccggccattcgctcgggtggagttcgtatccggtgtattcgcccggcgcacggcccgtcagccggaattccacgacggagtcaccggaccggtgccagacatagcgcgggaagccgtcctgccagggaccgccgaggaatccggtgcggatccctgaacggagggtgcccagcggggcggccgacagggaaccgggcacggcgagcagatcggccggggccgggccgtattcccggcgggcgaatgtcacccgcccgcagagttcttcccgttcgctgcggtcgggcagtggcgcgatcgcgcgacgtggctcgcggcggcgcccgggcgctcgtaggaccatgatgtccgcctttcggggaacgtgccggtgagctgggccggcggggcccggacgcggcgtgcgtccgggccccgcccagggtgttacgggaggggcgcgaagaggtccaccacgttgccgtcggggtccttgacgatggcgtagcgctgaccccacacggcgttccacggcttgaggtggccctcgtagccggcgtcgacgagctcggcgtacttcttgtccacgctcgcggtgtcggggaactcgaacgcgatggcgaagcggtggccgccggtgggggcctgccactcggggtcgtagctgcgcaccgtctccacggtgtcccaggcgagccggatgccgccgtcgagcacggcctccgtgtgcggcgcggagtcggcctcggcggggatctcgacgcccagcttccggtagaactccagcgacttggccatgtcctcgaccaccacggcgaagagggaaatccttgctgacatgcgcgttcctttcttgcacttttaaattggtctccggtgccgggccgtctgaattctccggggccggccggaccacgaagtccgaatgtgctggacgcgccgtacgctagtgactgcgcgctgactttggccaatcggggtatcccccgccggagtcaacgccgctgacaggacaacgatttcaggacagcggcacgccgtcccagtcgttcggcacgtcacgcccgagcagagcgcatacggtcgcgggaatatcgacgggggcggcggtgcgggtctgccgcgccccaccggtgaagccggggccgacggccgaccagtaggactcccggcggtgccagccgctctgccagtcgcggtggacgtgcgaggcccagtgcgggtcgcccagcggaagatagcgccagtccgccggctccaggatgaggtcgggggcgtgctgggtggcctcgccgggatagacctcctcccggcggcgcaccgcgtcgaagaacagcctgccggtacgggggttgcgccgctccagcagcgcggccgcgacgtcggtgcggaccttctcgtagtcgcgctccgggaccaggccgtgcttgtagcggtcgcgcaggttgatgttcaccccgtgcgtgccctgcaccgcctcgaaggccgcgctgccggcccactcgacgcggccgtcctcggcggtggccaggaaacccgcctgctccatctcgtcgttgatggaacagtagttgcgcagcggcccgaagcctatctccgagaaggccacgacactggtgcggtcgtcggccgcccgcagggcgtcctggatgacctggtcgcaggtgcggtaggcggcgaagacggcgctctcccgctcgtgctcggggccgtgctccagctcctgccagtagatgtgcgaacagcggtcgatgctcgtgaggttgacgatcacgacatcggactcctccagcagagccaatgccgcgcgcccgcgctgcacgtccgcctccagcagggaaggcagcagctcgtcgcggtcctgcccggtccagaagatcgacacgtcgtggaccggacggatgcccttcttcgccagggtgcgctggaggctgcgcgggtggcaggcgtggagggtggcatacatcggataggtgatcagggaaccgtcgaagggctccgggggatgggtgccgaagaggcctatcgaggcgaacctgacgccctggaacacctcgtgctgccacagcagtgggtggcggcggtgctcgggggtgaggacctgcggcgcgtactccgggtcgtgacaggtccagtaggagtagaagccgtggtccgcggcgcgccggccggtcaggacgctcagcaggcccggcggttcgtagggggtgccctcggcgtggagcggcccggaagccccctgcgagcgcagggcggcgaagccgggcagcagcccctgggcacaccagcggtcgagcagctcgggtgccgctccctcggtgatgacgacgacgacacgctggcggattgtcacgtgcgactccctcgggttgcgtggcagttggcatgccgtcatccgggaggcgccggaaaggccgaggcgttccggcgccggacaggcgtcgatcgtcggatcaagctaacagcgggacgaggactctctccagacgacggtacggaggaaattgagagagggctgagagagggctgagagagggcagaggcgggggagtggcgtggggtcacacggtgcgcaggaggcgcagacgttcccgtaccgccttgggcagccccgccaccacgcgatcgtacgaatgctccaccatctcccgcagctcctccacgggaaccgtgccgttcaggacaaccgtgttccagtggcgcttgttgacgtggtagccgggcaccaccgccgcgtactgctcgcgcaggtgcagcgccagatccggttcgcacttcagcgtgacctgcggcgggcggtcctcggaggcgtcctggagaatggcgaagaccttcttctccaccttgaagaccgcggctccggggccgaacgcctcgtcgtccaccgcctccggcagctccagcgcgaagtcggagagttcctctggtgtcatcgccggtccttcttcctgcggcacggcagcgagcggccgaaccgcgtggtcatggggtcggccaacagactagaggcgcaggaggagttgccgtgcggcagggcgcggacgctgatccacgatggccgaaacactgcggggagttccggtcgcggcgggacggcgaccttgacgggcggtcctgccattggcacagtttggctggctccacacaggttttcggtggaccgttcgttcctctcccggtgctgcccggtcgcggtaccggtgtccgcgcgatccgtgtgccgcccgcgccgtcccgaaccggcccgtgcgcccactctcccggccctccgccgccggtctccgtaccgccgccccgcccttgccggggcggcgccgacgcccgcaccccggccttggccctgcccacggccgcatccgcgcacccccctcaccccggcgccggccatgcccccgtgccgcctgccccccttgatgccctgtggaggaacccccgtatgaccgtggagcagacccccgagaatcccgggaccgcggcccgcgccgccgcggaagagaccgtgaacgacatcctgcaaggggcgtggaaggcccgcgccatccacgtggccgtcgaactcggcgtcccggaactgctccaggagggcccccgcaccgcgaccgccctcgccgaggccaccggcgcccacgagcagaccctgcgcagactgctccgactgctcgccacggtgggcgtcttcgacgacctcggccacgacgacctgttcgcccagaacgccctctccgccgtcctgctgcccgaccccgcgagcccggtcgccaccgacgcgcgcttccaggcggccccctggcactggcgggcctgggaacagctcacgcacagcgtccgcaccggtgaggcgtcctttccttcgacgtggccaacggcacctcgttctggcagctcacccacgagggaccccaaggcgcgcgaactgttcaaccgcgccatggggtcggtctccctcaccgaggccggacaggtcgccgcggcctacgacttctccggcgccgcgaccgccgtggacatcggcggcggccgcggcagcctcatggcggccgtcctcgacgccttccccggcctgcgcggaaccctgctggagcgcccgcccgtcgccgaggaggcccgtgagctcctcaccggccgcggcctcgcggaccggtgcgagatcctgcccggcgacttcttcgagaccatccccgacggcgccgacgtctacctcatcaagcacgtgctgcacgactgggacgacgacgacgtcgtacgcatcctccgccggatcgccaccgccatgaagccggactcccggctcctggtcatcgacaacctcatcgacgagcggcccgccgcatcgacgctcttcgtcgacctgctgctgctcgtcctcgtcggcggcgccgaacgctcggagagcgaattcgccgcgctgctggagaagtcgggcctgagggtggagcgctcgctgccctgcggcgccggcccggtgcgcatcgtcgagatccgcagggcctgaaaccgcccctcctgaccgaagccggccacagctgaaggagcaatgacaccatgacggtgctgggtctgggtggatccggacatgactgggcctcctgtgccaccgacggccgacggctggtggcgatcgacgaggagcggctggtccgcagcaagtacggcctgggagcggacctcctggcgggccacagccggcgcgccgtcctcgacgccctcggcacgagtgccgaggccgtggaacacgtggtggcctgcgagctcgtaccacgccccttctaccactcgttccgcaggcgcgtgacggtcgtcaaccaccatctcgcccacgcctacagcgcgttcggggcctccgggatgacccgcgccgccgtactggtctgcgacaactccggcagcctggtgacgggcctgaagtccggcccagggccgcgcgaggcggagacgatcagctgctacaccgccgacgcctccgggctgcgcctggtcaaccgggtcgccgggacacacgccgtggacgcctcctccgagagcgcctactaccagcccggcgagaccgacaattccctcggccacttctaccgctcggccagcctcgcactcggcctcgcctactccggtcccaagacccgctaccccgtcagcgaggacggcaagaccatgggcctcgcgccctacggcgacgaccgcttcgtcgacgaggtcgcggagctggtcaccctgctgcccgagggcggcgtgcagatctcggcgagcaaggtgaaccacctcttcgaacgcctcgtggaatcgggtgagttcgaggaccgggcggccttggcctacgccgcccaggagacgctggaacgcgccctgctgcactgcgcccgcgacctgcaccgccgcaccggcctgacggacctgtgcatcgccggcggcgtcggcctcaacagcgtcgccaacggccggatcctgcgcgagacccccttcgagcgggtcttcgtcgtcccggccgcgggcgacaacgggatcagcctcggctgcgcctactacggcctccacgagctggaggggcgcgcgccgtcggagctccccgccctcgacaccgcctacctcgggcccgactaccccgccgagcgcgtcgacgcggcgctggccggctcgggcttcaccgtggagacccccgacgacctgcccggcagggtcgccggcctgctcgccgaagggaagatcatcggctggttcgacggccgctccgaattcggcccgcgcgcactgggacaccgcagcatcctcgccgcacccttccccgcctccgtgcgggaccacctcaacgacaacgtcaaacaccgcgagtggttccgcccctacgcccccatcgtccgcgaggaccgggcggcggactacttcgacctcgtccagccctccccgttcatgctggtcgtcgcgcgcgtgacccggcaggacgccatccccgccgccacccacgtggacggcaccgcccggctccagacgctgaacgccgcacagaacccgaaggtctacgagctgctcggcaggttcgaggcgctcaccggctgcgccgtgctgctcaacacctccttcaacgtcgccggccagcccatcgtcgagaccccggaggacgccgtcgaggcgttcgcgggcatgcgcctggaccacctcgtcgtgggggaccggctggcgaccaagccctgacagcacgccgaggcccgcgaccggcagggaggagagccaagcggtggacgtccccgtgctcgtggtcggaggaggaccgacgggcttggcgatggcgctcttcctcgcacgccacggcgtcggctgcctgctggtcgaacggcggacgaccacctcgcccgtcccgcgcgccacccacgtcagccgccgctccatggaactcttccgcgaggcgggcctggaggaggagatccgccgggccgggttcgaggtcgtgcgcgaggacgacccacggctgcggacccggcccgaacgccacctgccccgggtggtcctgcaagccgcctcgctcgccggccccggcccggtgggggtcctggagaccggtgacgaggaactggccgtacccggcccctgcgcacccttctggtgcggccaggaccggatggaacccctgctcgccaaggccgcggcgcgccacggcgccgatgtgcgcttcggccacgaactgaccggcctgtggccgggggaggacagcacacgggcccgcgtccgggcagcgggaacgggacggacctacaccgtcgacgcccgcttcgtcatcgccgccgacggggcgcgcggcgagatcgccgagcgcgtgggcatcgcgcgggagggcctgggcacggtcgcccaccgggtgagcatcctcttccgcgccgacccggggcgctgggcccgcgaccggcggttcttcatgtgcatgatccagaacccggggttcgacggggcggtgatggagctcaacaccccgggccgctggtgcgccgcggtggactacgacccggcccgcgccgaacccgacggcacctactccgcacgcacctgcctcgacctggtccgggccgccgtcggtgacgaccggagcgacgcggcggtcgacaccgtcttccactggaaggcccggcaccgcatagcggccgcctaccgcagtggggcggtgttcctcatcggcgacgccgcccacctccacccgccctccggcggctacggatccaacgtcggcttccaggacgcgcacaacctcgcctggaagatcgccgccgtgctcggcggctgggccggaccgcggctgctggacacctacgacgaagagcgccgccccgtgggaaaggcgacggcggagcagtcgatgctcctcgacggcgtgccaccggaaccactggggggaagcgtcgtccgctgcgatccccgcaccctgatcatgggataccgctaccactccgccgccgtcctcggccccccgcacggccccgccttccccgcggccttcaccctgcgcggagacccgggcacccggctgccgcacgtatggctgcgtacggacgcgggggaacgcgtctccacgctcgacctgtgccacgggcacttcgtcctgctctccgccgacccggtctgggcggcggccgcggcgcgctcggcgaaggagacgggcgtaccgctgcggggccaccacctggcggccaccggaagcgaactcgccgacccctccggcgagttcccgcggagctgcgggaccgggcccgcgggggccgtgctcgtacggccggacggcatggtcgcctggcgcacggcccgcgccgtgcccccggacccggacagcgcgcaggacctggtcacggcagcggtgagacgtgtcctcgcactgccggagcgcgcggcgccaccggtgctcggtccgccgcggttgtcacgcggttcctatcggcgagtcgggagcgacgggtgaagcctcattccttctgcacgtgctggccgggcgccaccgtatggctgacgggcccaccgggcgcgggcaagacgacgatcgcccgcgcactggcggagcggctgcgcgaacggggccggcgcgtggaggtgctcgacggcgacgcgacccgcgcgctcctgaccgcgggctcctcgtgggaggaccgtggcaccggcctccagcgggtcggcctgatggccgaggtcctggcgcgcaacggcatcgtcgtcctcgtcccggtgaccgcggcccgcgcggacagccgcgaagccgtacgcagacgccacgagcggtccggcaccgcgcacctggaagtgcgggtggtccgggacgcagtgcctccgagcgggctccccgcgccgcccggcccagatctgcggatcgcggcgcacgagcagagcgccgaggagtcggcgcgggcactgcaccggctcctggcggagagggagctggcgtgaaccccgggcgcggtggagcgtacgccgcggggcgcgacgggacccgcgggacgcgacgccctcacggtctgtcgcacctggatctgctggagtcggagtcggtccacatcttccgtgaggtggcgggcgagttcgagcggccggtgatcctcttctccggcggcaaggactcgatcgtcatgctgcacctggcgctgaagtccttcgctcccgcacccgtgccgttcgcgctgctgcacgtggacaccggccacaacttccccgaggtgatcgcctaccgggaccgcgtcgtggcggcgctcggtctgcggctggaagtggcctccgtgcaggacttcatcgacaacggcaccttgcgcgaacgcccggacggcacccgcaatccgctgcagacggtgccactgctggacgcgatcgggcgccaccgcttcgacgccgtcttcggcggcggccgccgcgacgaggagaaggcccgcgcgaaggagcgggtgttctccctgcgcgacgagttcggcggctgggacccgcgccgccagcgccccgaactgtggcggctctacaacggccgccacgcacccggcgagcacgtccgcgtcttccccctctccaactggaccgagctcgacgtgtggcagtacgtcgcccgcgaggagatcgaactccccaccatctactacgcccacgagcgcgaggtcttccgccgcggcggcatgtggctggcaccgggggagtggggcggcccacgcgagggggaagcggtggagaagcgacgggtgcgctaccgcacggtgggggacatgtcctgcaccggcgcggtggactcggcggcggccaccgtggccgacgtcgtcgccgagatcgccacgtcccgcctcacggaacggggcgcgacccgggccgacgacaagctgtcggaagccgcgatggaggaccgcaagcgcgaggggtatttctagcgcggcggggccggtgcggcccacaagcggaggactagtccctaagtatgaagtcccctactccgtttgtctgttgagggcaggggcgccgtctgaggatgatgcagtccatgtcacagttactttccgggaaggacggcgcccaggaggcgccaagtcgcggcgggtccacgtgggtggcggtcctcgccgcgtgcgtggggcagttcgtggtggtcctcgacgtgtccgtcatcaatgtcgcgctgccgtcgatccgttccggcctcgacatcggcgagacgggcctgcagtgggtggtcaacgcctacgtcatcgccttcgcgggcttcctgctgctcggcggccgggcctccgacctcttcggccgcaaggccgtgttcgtcttcggcctcggggtgttcaccgccgcgagcctgctcggcggcctcgcgcaggcgccgtggatgctcatcgtcgcccgcgccctgcaaggcatcggggcggccgtgctctcacccgccaccctcgcgatcctcaccaccacgttccccgagggtccggcgcgcatcaaagccgtcgcgatctggacggccgtgggcacgggcggcggcgcggccggcggcctcatcggcggcctgctcaccgactacctctcgtggcgctgggtgttgctgatcaacgtgccgctgggccttgtcgtgatcgtcgcgaccgtcgcctggctggccgagagccgcagcgaccaggcacaccgacgccggctggacctcccgggagcggtgctggtgaccctgggcgtcggcagcctggcctacggcatctcgcagagcgagggccacggctggggctcgccgcggacgctcaccttcctgatcgtcggtgtcgtggcgctcctcgccttcgtcgccgtggagcagcgcacgcgcgagccgttgatgccgctcggtgtcttccgggtgcgctcggtgtcggcggccaacgccatcaccatcgtcagtggcatgggcttctacgcgatgtggtacttcctctcgctctacatgcagaacgtgctgaaatactccgccgtacagaccggcctggccctgcttccccacaccgccaccatcatcctctccgcgcagttcgcaccccgcctgatgcggtggatcaaggggcgcaccctcctcgtgatcgcgggactgctgaccgccgcgggcttcatctggcaggggaacatggacgccgacggctccttcctggcgaccctgctcggcccgggaatcgtcttctccttcggcgcgggcctgatgatgacgctcctcgcggtctccgccacgacgggcgtggagctctccgaatcgggcctggtggccggcctcgccaacacctcgcgcaccatgggcggcgcgctcggcctgtcggtcctcgcgtccgtcgccgcccgccgcacggccgacgtggggcccggcgcggagggcctggcctccggctacggtcgggcgttcgtcgtgtccggggccatcatcctcgtgagcatgctgatgatccccttcctgcccaagccccagccccagaccccggcggaatgacctgtgagcacggacatacgaggaggcttcgtggggcaggacagccggccgcggtggctcaccgacgaggaacaacgcgtgtggcgcggctatctgcgggccaccaggctggtggaggaccacctggaccgccgcctccagcgggaagcggacatgccgcacctctattacggtcttctcgtccagctctccgaggccccgcgccgggggatccggatgaccgaccttgcccgcaacgcgaagatcacccgcccgcggctctcgcacgcgatcacccgcctggagaagctcggctgggtgcgccgggaatcgtgccacggcgacaggcgcggccagaacgccgtcctcacggaagagggccgcgaggttctggagaagtcggcgccgggccatgtcgccgctgtgcgcgcggccgtcttcgacagcctcaccccggaacaggtcgggcaactgggccggatctgccaggcgatagagaaggggctggaccgggaaggcgcggacctgccgtggctgcgctgaggcgggaagccgtcgcgagcgcgcggggccgtcaggctctgacggcccccgccgcccgcgtacgggatcgggccgaccgcgccccggattcacgcgagtccgggagcagaccggacgacacggatattctggatgccgtggaacgacacgacggggcaccgggctggggcttcacccatacccagtacagcgcggaccacggtgaacgcggcgccacccgcagggccggggccctgctctccgcgcggcccctgccgcagaaccagcacatcatgggctggggcgcggagaatcccgaaccggcgcccggacgctacgacttcgaggtcctcgacgagcgcgtcgccctgatgcgcgcgacgggggccacgcccgtcctgaccctgtgtgccgcccccgactggatgaagggcggccggcccggccgcaccgactggtcgcgactggagaccgcccccgacccccggcactacgcggacttcgcccggctcgcgggcgtgatcgcccaacgctacccggacatcaggcacttcctcgtgtggaacgagctgaagggcttctacgacgaggacaggcggcgctgggattatgagggatacacccggctgtacaacctcgtccacgccgagctgaagcggcggaacccgcgcaatctggtgggcggcccctatgcggtggtcgaccacgacccgcccgccgaggacgcggcggaccgctcgcgcgaactgcgcggtccctggggcgagctggaccagcgctccgccgacgtcatccgctattggaacgcccacaaggcgggcgcggacttcgtcgtcgtcgacgggtccagctacacccgcgagggccaccgggcgattccggacgagttcgccgccaccgagaagttcgccgacgtcacccgctgggtcaggagcgtgaccggactcccggtgtggtgggccgagtggtacgtcgagccgcccgccgaggacgaccggccgggcggccgggacggctggggcgaggggcaccgcaccgccgtgcaggccaccgcgatgatgcggctggcggagagcggcgcgtcggccgccttctactggaacccgcagcggaccgggaaggcgtgccccggctgcctgtggcggagcacccacttgcgcgacgggggaggggagttgcccatggcgggtctcctgagccggttcgctcgcgaattccctccgggcaccgccttccggccggtcgccgtcacctgcgggagcggtgacagggtcgaggccctcgccgacgaggccgccgtgctcgtcgtcaacaccgagtgccggccggtggccgccagggtggacgggcaggcgctgtccctcgcgccgtacgaggtgcgctggctgacccgcccgtaatccagtggggcggcgcacgggcgcggacagggaattgcggaacagggaagttcacgaataaggagaacgcgggaaagcgctcgggcggagcgtgaaacccctgtcggcgctcacgatatccacccagctgatttgcaggtgaaacgggcggtcgcctcgacggtgccgcccgtttcctgttgcccgaaagggcaatcgggcatcagcaggagagattgccgcccggcgccacgccgaggatcttggtgaatcgctggtagttcgcgacgcggctctggacctgggcggggttgtggccgtcgcactccagggcgccgttgatgctgcggatggtctgcccgaagccgcggtggttgaccatggcctcgtgcggggtcatggtgccggggccgcgctgggtgttccagtaccacaggccggtcttccaggagacggccgcgtccttctgcaccagcgaggggttgtggagcaggtcgatgccgagggcgtcacccgccgccttgtagttgaagttccagctgatctggagcgggccgcgaccgtagtaggcggcctggcctgccggacagccgtagggccggctccggtcgcagtagtgggggtagttggcggtgttctgctccacgacatagaccagtccgccggtctcgtgggcgacgttggcgaggaaggcggcggcctcctgcttccggacctcggcgctgccggtgcccgcgaagcccgggtacgccttgagcgcggcgaccaggcccttgtacgtatagaacgcgttccgcttcgggaacatctgcttgaactgggcctcgctcacggggaatgcggcggcctgcgaggtgccgccgtgcggtgcggccgcgctcgccgtggtggcgggggcgaggacggatatgccgaccagtgccagagcggccggcagcagggcggcgatgcgatttcttctcatggcggctcccgtgggggaaagggtgagtgacgcccgccgacggtgaatcgggcccgttgggcgccttcgcgtcatcgcgcagtgaataactcccgtgagtttggtgtcaatggcatgcgccgtgtccggccgaaccaggtgcactgagcaatgagttcaggacaactgcggccgatagggcttgcgggagcaacgaggaccatgacctcatatgccggaagccggacacgtgccgagaaatgccgctgtcctgtggctccttgggtgacctgtgaaacccggctggctcatgaacgagccgattgaacgagccgattgaacaagccgatgaacaagga

SEQ ID NO: 77-78 - skipped (2 entries)

SEQ ID NO: 79 - DNA, 621 bp, Streptomyces lavendulae
gtgccccggaatctcctggacgtcagggacgtccaccacacctacggcacccgcagggtgctgcgcggcatcgacttgtccctgcgccccggaacgctggccggcgtcgtgggcgagaacggcgcggggaagtcgacgctcctgaagatcctcgccggtgagctgcggccgcagcgcgggcaggtccactacggcggccggttcggttactgcccgcagcatctcgtcctgaaccaggctctcaccgtccgccagcacctggagtatttccgggtggcgtacggcctcgccaccctcagccatgccgagagaatcatggacgtgctccggctgtccgactacggggacgagcgggtcagcgtgctcagcggcggcacgaaacagaagctcaacctcacactggccctgatgcacgaccctgacctgctcctcctggacgagccctaccagggcttcgactgggacacccaccagcggttctggagcctggccgccggtctgcgcgaacgcgggcggtccgtgctggtcgtctcccacctggcctacgacgccgaacggctcgacgaactccggcacctcgacggcggactgctgcactcaaggacgacggcaccggtatga

SEQ ID NO: 80 - DNA, 1350 bp, Streptomyces lavendulae
atgaccggcgccctcctctgtcccagggacatctccccgactttcgcggcgaacgcggacttcatccggcagcgcatcgaccgcacggccgaacgcatcaaccatcacatcgaccgcctgtgcccgaacgcgtcggcagatgcttgctccacgtggctgccaccgggccacatcaccgggacgaccgggcctggcacgccgccggtcgtcgcccagcggctgcaccgggccctgacctctcccgtccgccatctgaccgatgccggaggacagcgctggcggccggtgctggcctgggaggccatcggtctgatgggaggtgacagcgaatcctgcggcctgctgatcgcggcgagtgagctgctccacaccggatccctcatcgtcgacgacgtccaggacgcctcaccgctgcgccgcggacaaccggccgtgcacaccatgttcggcatgccgactgcggtgaacgcgggtacggccgcctatttcctctgggagcgggccgttcagctcacctttcccgacgacgcctcgcggtgcggggagttgcgggcactgggtctggccgcgctgcgagcggctcacgccggtcaggcactggatctccaaggtcaccgggaagagatggaccaggccgtggccggcgacgaccggcacactgtgctggaactggtccgtctgacacaccggttgaagtccggggccccggtctcggcggccatggaggcagcaggggtcgtcacgggtgccgagccggaactgcggagagcactgggggctttcggttcagcggtgggcaccgcctaccagatcgccgacgacgtcgccgacctgagcggtgtcacacgggcgggggcaccgacgaagcaggccaccgaggacctgcggagcggcaaggtcaccatgccactggcccacgccgtggtccggctgccgcagagcccggctgaaccagctctggcagcaggtcaaggacggctcgggcagcgcgacggcggtggccgaggtgtgccgggacctgaacgcctgcggagcggtgagagcctgcctggaggaagccgaccagttggtgagcgacgcgtggaacaagctcgagcccctgctgccgtcggcaggccacagccacttgttgtacgagctggccctctcggtcgtccggcgcgaccaggtcgcctgaggagaggaagccgtgccccggaatctcctggacgtcagggacgtccaccacacctacggcacccgcagggtgctgcgcggcatcgacttgtccctgcgccccggaacgctggccggcgtcgtgggcgagaacggcgcggggaagtcgacgctcctgaagatcctcgccggtga

SEQ ID NO: 81 - DNA, 1425 bp, Streptomyces lavendulae
atgcgggcgggaggacgcctgatgaacggagattcgaaggcggactccgttgtgccgatggctcctgactgcagtcggttgctcgggcatgcccgctccgtgcggttcttgatggatctgagggagatcgcggcccggctcggcaaggcggggcctgtggtcagggtgaaggcgggcccgttcgtcgggtacctgctcaatgactcctcgctgatccggcaggctgctcgcgacgaggacaccttcatgttctggggcagggaaccccatgtgcgggtgatcgtaagagagggactgctgagcaccgagggaacggtccaccgtgaccggcgggcggtgatgaggcccgcgttcgccgtgccccggccggcggacctcggggcgtccgtgcggaccgagacccgaagtctgctcgccacccttcccgcggaccgtccggtcgacatgagccacgagatgacccggctcactttccatctcgccgtcaggtgcgtcttgcgcagcgaggtctcgcccggaacgctgaccgcgctggccgcggcgcatgccacgctgtcgcaggtcggggccttgcggtttctcctgtccccgtggccttgggtacccgtgccaagacaacgcgcgctccgccgtgccctcgcggtgctggacgaggccacccgggaagtcctcgcgcgccatcggccggctgaggacgggtgtgatgtggtctccctgctgaagcaagcgtggcgcgagccctcggaagcggcggtgcaggacgtgcgcacgctgctgttcaccggcggggaggccaccgcctcgacactcgcgtgggcctgctacgaactgggccgccacccccaccatcaacaggccttgcaggaggaggccgacgcggcactcggcctggggcactcgcaggccggcgtcggaataaaccggttgccccggacagccgccttcgtcaaggaagtcatccggcttcacggcctcccggtccttgtgcgttgcacgcgccgcgagacgtggctgggcggccaccggctgcccgcgaaggcgacggtgttcctccacctcggggcgatgagccgtgaccccgcccacttcgaacggcctgatgtcttcgatccgacgcgctggatgccggacgctcagccctcggtctcccctgctgcctggcttccctatgcgctcgggccccgctactgccctggtgccgcggtggccgacgtcatggtgcccgtcgcgctcgccactctggtcgccacgcggaccgtacgcacggcgcgtccgggccggacggtgcgctccggcttcgaactcgccgcgatgccacgcggcctgaccatgatcgccgcactccgcgagccgtgcccgagcagtccctccgcggcatcgggccggcgaccccatgagggagcctcgtccatcgcccgcccctga

SEQ ID NO: 82 - DNA, 1821 bp, Streptomyces lavendulae
atgaagatctctcgaataggccgcgcgtcatccatcgccgccctggtgacaaccgcactcgctttcacggcagttggcaccgtcgctcccacggccgtcgccgactcccgcgcggccgccgcttccgggacgcagaatgaccacccgagctcggggcagggcacctccacctctgagctccggcgcaagggcctggtcccgtcgagtctcgtggccaagcccatcacccgcagcgagaccctcagacgcgccgccagctggttcggcaagggtctccactacagcggggacaacacctatcagggctggcgcacggactgctccggcttcgtctccatggcctggggactgcccggcccgggtgagaccaccgattcgttcattcccgggggcgtggcccacgaaatctccaaggacgaactgaagcccggcgacgcgctcaacaacaaggcgctcggcaacgacggtcacgtcgtcctgttcgagaagtgggccgattcctcccagtcctcctactggggttatgagttcagcagcagcggtctgcaccaccgtgtgatcccgtacgcctacttctccaggtccgagcagtaccgcccgatccgcttcaacaccatcgtggacgacgacacggccgcagggcccgccgaggacaacgcccgggtccagggtgacttcgacggcgacggccgcgacgacgtggcggtgctctacgactacggcaggaaggacgaccgcagtcgctcggccctgtggacgttcaacagcaacggcagcggtttcaacagtcccaagcaggtgtgggacagcgggacgtcggagagctggaactgggcctccagcaagttgacggtcggtgacttcaacggcgacggcaaggccgacatcggcgtcctctacaacatgggcgcgaccgaggacggccgcaaccgcaccaagctgttcgtgttcaccagcaccggcagcggattcgccgccccggtcaaggtctgggacagcaacgacgaccccgtgaagagctggaactggaacgccagcaagctcaccgtcggcgacttcaacggcgacggcaaggccgacatcggggtgctgtacgactacggcaaggacgacgaccacaaccggacagggctctggacgttcaccagcaccggcagcgggttcaacagcccgaagcaggtgtgggacagcaacaacgaccccgtgaagagctggaactgggaagccagcaagcccgtctccggggacttcaacggcgacggcaaggccgacatcggcgtcctctacgactacggcaagaccgactccggcagccgcaccggactctggacgttcaacggcaatggcaacgggttcaacagcccgaagcaggtgtgggacagcaacaacgaccccgtgaagagctggaactgggaagccggcaagcccgtttccggcgacttcaacggcgacggcaagagcgacatcggcgtcctctacgacatgggtcgcaccgaggacggccgcaaccgcaccaagctgttcaccttcaccggcacggcgaccggtttcaacagcccggtcaaggtgtgggacagcaacgacgaccccgtgaagagctggaacgcgtccgcgagcaagcccgtcgcaggtgacttcaacggcgacggcaaggcggacatcggcgtcctctacgactacggcaagaccgactccggcaaccgcagcggactgtggaccttcaccagcaacggcagcggcagcgacagccccaagcttggctgggacagcagcgcggaccccgtcaagagctggaactggagcgcgagcaagctcggctga

SEQ ID NO: 83 - DNA, 2466 bp, Streptomyces lavendulae
atgcgaaccatacgaatacgaagaacgaacggcgtggccttcgccgccgctgccgccctgatggccctcgtcgcctccggcaccgccacggtccaggccgcgccctcgcacgccggaccctccggcaccactccgatcacctaccgtggcctcaccctcgacataccctccgggtggccggtcgtggacctggagaaagacccgcacacgtgtgtgcggttcgaccgccacacggtgtacttgggccaccccggcaccgaacagtcctgcccctcccatctggtcgcggacaagacggacgccctgatattggagccgatcaccggagcgggcggccaggacgcctcccacgcgctgcgcatccctgccggggccccgatgccgcacgagctgccggtgacgtacgaccacgagacgaaggtcgccgtcgaaggcgccggagtcatggtcacgtcctcctacggcacgtccagtacaacggtcgccgccgtcctcggctcggcccgcacggacgcgacagccaagccgacccccctgcccggcaaggcgggcaggggcctcgcggctccaccggttgccgccgtcgcggccgacaagggatacacagggctgggcttcgagtcctgcaccgccccttcgtccgccgcgatgaaggcatggaaggcctcgtcgccctacggggccgtcggcatctacatcggcggtcgcaagcggggctgtgcgcaaccgcagctcaccggcgactgggtgcgtcagcagaccgccgacggctggcacctgctgcctctcttcgtggacctccaggccggcgacatctctccggccaccgcggacgcgcagggccgcgagtccgcggacgccgccgtggccaaggcggcggacctgggcctgggccccgggacggtcatctacagcgacatggagcactacgacagccgctcgtaccgggcccgggtcatcgactacgtgtcggggtggaccagccgcctccacgaacatggctaccgctccggtgtgtacgcgggtgaaacgagcggcatcccggacctcgcctcggtggccgacgacaccaactacgcatcacccgacgtgctgtggtcggcgaactggaacctcaaggccgatgtgtcggacgcgtcgatgggacttccgggccccggctactggcccaatgggcggcgcatccaccagtaccgcggccaggtgaacgacacctacggcggtgtcaccctcgccatcgaccgcgactacgtcgatgtcgccgcggactcggccctgcccgcacccggcggagaggacggttcctcgcgcgtcaagggcgacttcgacggcgacggccgcgacgacgtggccgtgctgtacgactacggcaaggagggcggcgtcagccggtccgcgctgtggacgttcgcggggaccggcagcggcttcggcgccccgaagaaggtgtgggacagcggatcggacagctggagttggtcggccgccaagctgacggccggcgatttcaacggagacggcaaggccgacatcgcggtcctgtacgacatgggtcgcactgaggacggccgcaaccgcaccaagttgtacgagttcaccagcaccggcagcggattcaacagcccggtcaaggtctgggacagcaacgacgaccccgtcaagagctggaactgggcctccagcaagctgaccgtcggcgacttcgacggcgacggcaaggccgacatcgcggttctgtacgactacggcagggacggcgaccgcagccgtacgggcctgtggaccttcaccagcaccggtgccgccttcaccggccccaagctggtgtgggacagcaacaacgacccggtcaagagctggaactggaacgccagcaagcccaccgtcggcgacttcaacggcgacggcaaggccgacatcggcgtcctctacgacatgggtcgcaccgaggacggccgcaaccgcaccaagctgttcaccttcaccggcacggcgaccggtttcaacagcccggtcaaggtgtgggacagcaacgacgaccccgtgaagagctggaactgggacgccgtcaaggtagtgggaggcgacttcaacggcgacggcaagagcgacatcggggtgttgtacgactacggcaaggacggcgaccgcagccgcaccggactgtggaccttcaccagcaacggcagcgggttcaacagcccgaagcaggtgtgggacagcagcaacgacccggtgaagagctggaactgggccgcgagcaagccggtcgcaggggacttcaacggcgacggaaaaacggatatcggcgtgctctacgactacggcaggaccgattccggcaatcgcaccggactgtggaccttcaccagcgacggcaccggattcggtacacccctcctgggctgggacagcgtgacggatgccgtgaagagctggaactggcgtgccagcaaggtgagttga

SEQ ID NO: 84 - DNA, 1575 bp, Streptomyces lavendulae
gtggatcccttgacgcgcaagacccgcaccccccgcaagaagggcagacgcgcgagcgcggcggcgatgtcggcctccggcatgctgctcgccttggtggccaccgccgcccccgtccccgcccaggcggcatcactcgccacctgggaaaagatggcccagtgcgagagcagcggggactggggatacaaccagccaccgtactacggcggcctgcaattcctggagagtacgtgggtggcgtaccacggaacggactatgcgccatacccctatcaggccaccaaggaacagcagatccgggtcgcgcagcggctcctcgacaatgagggcgcggctccctggccgtactgcggaaagaaggtggggctggctgacgacgacgcacgccccttccccgacgcgccggacgacgacgcctccgcccggatcaacggtgacttcgacggcgacggatgcgacgacgtggccgtgctctatgactacggcaaggagggcggcgtcagccggtccgggctgtggacgttctccgggagcggtaccggcctcggcagcccgaagaaggtgtgggacagcggatcggccagctggagttggtcggccgccaaactggccgtcggcgatttcaacggcgacggcaaggccgacatcgcggtcctgtacgacatgggccgcactgaggacggccgcaaccgcaccaagttgtacgagttcaccagcaccggcagcggattcaacagcccggtcaaggtctgggacagcaacgacgaccccgtcaagagctggaactggaacgccggcaagctcaccgtcggcgacttcaacggtgacggcaagaccgacatcggcgtcctctacgactccggcaagaccgactccggcaaccgcaccggactgtggaccttcaccagcaacggcactggattcaacagcccgaaacaggtgtgggacagcaagagcgacccggtgaaaagctggaactgggccgcgagcaagccggtcgcgggcgatttcaacggtgacggcaagaccgatatcggggtgctttacgactacggcaaagatggcgaccgcagccgcaccggactgtggaccttcaccagcacgggcagcggattcaacagccccaagcagacctgggacagcgggtcggaaagctggagatggtcggcggccaaggtggtcggcggcgacttcaacggtgacggcaaggccgacatcggggtgctgtacgacctcggcaggaacggcgaccgcaaccgcaccgaactgttcacgttcgcgggcaacggcaccggcctcaacacaccggccaaggtgtgggacagccaggacgacagcgcggtgaagagctggaactgggccgcgagcaagccggtcgcaggtgacttcaacggcgacggaaagacggatatcggcgtcctctacgactacggccagaccgactccggcaaccgcaccgggctgtggaccttcaccagcgacggcagtggattcgccggccccaagctcacctgggacagccggaccgaccccgtcaagagctggaactggaacatgagcaagaccggctga

SEQ ID NO: 85 - DNA, 2175 bp, Streptomyces lavendulae
atgaaataccgaccgggaacactgctcacttccataacagtcttgtgtgccctgctcgttccggtgcgttcggcggctcaggcggccaggcccgagcagggacgttccgtggtggccgcggccgccgtactggagcaaagtccgccgacgctgctcgccgagccggaaatgcgcgtcgtctcctggaacatctgcggtgaggcgggcggggtgcgcggggagggcggctactgcccctaccgcaacgatccccaggcgaaagtcgaccagatcgcgcaggtggtcgcggagcgcagtgccaatgtcgtcatgctccaggaagtgtgcggcgaggcgcccggcagccatatggagcggctgcgcgcggccctgggcagcggatggtcgatcgcgcacgccccgggggcccgcccggacgacggaaccacgaactgccggggcgggctcagcggcatattgggcgtggggatcgcggtgaaggggcgcgtcaccgacaccaccgcgacgaacaccgtgcccgggggcggcggtgacaagcagaccctgcccatcctctgtgtacgtgtcgagggctggtcgtccaggatctgcaccacccacatcctgtccgaccctgccgatccgcgcaggccggggcagatccagaacgtcaagaacgagatctggccggaccgctatcagctggtgctcggcggcgacttcaacatgttccccgactccgccgggctcaagccgatctcggacgaattcgacgagtgcgaccgccgctcctacggcgccggtgacatggtcaacgaggtcacccatcactcctgggagaaaaagggcggacacatatggcgcaagcgtgaccacatcttcgcctcgtggggagagtccgggagccagttcacatcctgcgaggtcgaccggacccggatggacaccaccgagaacgcgcccgaaagcggtccgcccaacgggtattcggaccatgcgccgatcatcggctacctcaagccgccgcggcacctgagcacgtccggggacttcgacggcgacggcaaggccgacctcgcggtcctctacgggcaggggaagaccccggacggccacaaccggtccagcctgtggatctcaggcggttccggtaccggagcggagaccggattcgccgcgccgcgcgaggtctgggacagcggtgccgacagctggaactggtccgcgagcgcgctgacctccggggacttcgacggcgacggcaagaccgacatcggcgtcctctacaactacggcagggacggcgaccgcaaccgcaccgcgctgtggaccttcaaggggacatcgaacggcttcgaggcgccccgcaaggtgtgggacagccacgacgacacggccgttcccagctggaactggtccacgagcaagctcgtcgcgggcgatttcaacggcgacggcaaagcggacatcggcgtcctgtacgactacggcaggaccgcctccggcaaccgcaccggactgtggaccttcaccagcaccggcaccggattcggcaagccccacctggcgtgggacagctccaccgacccggtgaagagctggaactgggccgcgagcaagccggtcgcaggtgacttcaacggcgatggcaagaccgacatcggcgtcctctacgactacggcaaccacaccgccctatggaccttcaccagcaacggcaccggattcgccggccccaagcaggcctgggacagcggaccggagaactggaactggtccgccgccaagccggtcgccggggacttcgacggcgacggcaggaccgacatcgcggtcctgtacgactacggcaggaccgcctccggcaaccgcaccggactgtggaccttcaccggcaccggcaccggattcggcaagccccacctggcgtgggacagctccaccgacccggtgaagagctggaactgggccgcgagcgagccggtcgctggtgacttcaacggggacggcagggccgacctcgcggtgatgtacgactacgggaacgcgaccaacggccgcaaccgcaccgcgctgtggtccttcaccagccgcggcacggacttcgccgccccgcgggcgaactgggacagcagcaacgccgctgaccagctgaaatcgggcgagctgagggcggctccgctcagcgggtcctag

SEQ ID NO: 86 - DNA, 27 bp, Artificial Sequence
  A primer
tcagaattcggatccgagggccggagt

SEQ ID NO: 87 - DNA, 18 bp, Streptomyces lavendulae
acctactgcctcgatgcc

SEQ ID NO: 88 - DNA, 15 bp, Streptomyces lavendulae
ctgatccttcaagcg

SEQ ID NO: 89 - DNA, 20 bp, Amycolatopsis mediterranei
gcgtccgtgctgcgcgcgca

SEQ ID NO: 90 - DNA, 20 bp, Amycolatopsis mediterranei
tgcgcgcgcagcacggacgc

SEQ ID NO: 91 - DNA, 6 bp, Streptomyces lavendulae
gaaagg

SEQ ID NO: 92 - protein, 80 aa, Artificial Sequence
  A conserved motif
GXXXDXXXXXXXAXXXXXXXXEDXXXXXXXXXXXXXXGXXXXXXXXXXXXXKXXXXGEGGXXXXXXXXXXXXXXXXXXXG

SEQ ID NO: 93 - DNA, 6 bp, Streptomyces lavendulae
ggaacg

SEQ ID NO: 94 - DNA, 30 bp, Artificial Sequence
  A primer
gggaattccatatgatgcagtccatgtcac

SEQ ID NO: 95 - DNA, 30 bp, Artificial Sequence
  A primer
gggaattcaagctttcattccgccggggtc

SEQ ID NO: 96 - DNA, 18331 bp, Streptomyces lavendulae
ggatcccgatcgtctcggacatgaccggcgaccttctcggcgcgcgggaggcccaggaccccgcctactgggtgtcccacatccgccgcgcggtgcgcttccacgaccagatccgccgtctgcagcgctacggggccggggccttcgtcgaggtcggcccggacacggtgctcagctcggccggccaggcgtgcctgacggaccaggcgggcaggagcgcgcccgtcctggtgtccctcgcgcacgccgagcgcgcggaggtgcccgcgctcctgaccgctctggccaccctgcacacccgtggcgtggccgtggactggcgggcgtggttcggcgacgggccgcgcgcggccggcctgcccacatacgcgttccagaagcagcactactggccgtcgggccccaccggttggcggtccgggcccgcccccgtacccctgccccaggccggaacggaggacgccgaaaggcccggtcgcgccgcggagtggcgggcgctgccgcccggtgagcggtacgacgcgctgctgcggatggtgcgcggcgaagccgccgccgtgatggggcacgccgggccggaggcggtggagccggagcgcggcttcctcgaccacggcttcgactcggtgatggccgtgaagctgcgcgaccgtctcgtggccgggacggggcgggagctgccgacgaccctgctgttcgaccaccccacgcccgcggccgtcgccgactacctgctggcggggacgggcgaggccgagacggcgccgtccgtgtccctgtcggaccagctcgaccgcctggaggccgacctcgcgcggctgccggccgacgaccggcagcgcgcccgcgtcgccgagcggctcaagggcctgctcgcggtccacgcgccggaccggggcgccgggagcgaggacgcgccggaccaggacgcgctggacacggcgaccgacgacgagatgttcgagctgatcgagaaggaactccgccgtggatgagaccaacgagaccaaactccgcgagtacctgcggctggtcacggccgatctgcggcgaacccgcaggcagttggaggaggccgaggacgcggcccgcgagcccgtcgcgatcgtgggcatggcgtgccgcttccccggggacgtggcatcgccggacgacctgtggcagctggtcgccgagggccgggacgccgtcaccgagttccccgccgaccggggctgggacgtcgacgccgtctacgaccccgagccgggcaccccgggcaggacgtacgcgcgccacggcggcttcctcaaggacgccgccggattcgacgccgccttcttcggcatcacgccgcgcgaggcgctcgccatggacccgcagcagcgcatgatcatggaggtctcctgggaggcgttcgagcaggcgggcctcgacgcgaccaccctgcggggcgaggacgtcggcgtcttcgtcggctccaacagcaacgactacctgatcaacgtgctcgacgcgcgggacgtcgccgagggcttcatcgggaccggcaactccgccagcatcctctccggccgcgtcgcctacaccttcggcttcgagggcccggccgtgtccgtcgacaccgcctgctcctcctcgctggtcgcgctgcacctggccgcgcagtccctgcggcagggggagtgctccctggcgctggcgggcggcgcgacggtgatggccacgccgaccgccttcatcgagttcagccgccagcggggcctggcccccgacggccgctgcaagtccttctcggcgaccgccgacggcaccacctggtccgagggcgcggccgtgctgctgctggcccggctctcggacgcccgccgcctgggctaccccgtgcacgcggtcatccggggcagcgccgtcaaccaggacggcgcgagcgcgggcctgaccgcgcccaacggaccggcgcaacagcgggtgatccggcaggcactggccaacgcacggctgacggccgacagcgtcgacgcggtcgaggcacacggcaccggcaccccgctgggcgacccgatcgaggcccaggccctcctcgccacctacgggcgggcccgcggcgagggcaggccgctgtggctgggctcgctgaagtcgaacctgggccacacccagtccgcggccggcgcgggcggcgtcatcaagatggtgatggccatgcggcacgggacgctgccccgcacgctgcacctcacggagcccaccccgcgcgtcgactggtccgccggtgacgtacggctgctgaccgaggcccaggactggccggacaccggacagccgcgccgtgcggccgtctcgtccttcggcgtcagcggcaccaacgcccatgtgatcctggagggcccgcccgccgaggaggcaccggacgcgccgctgccggacgtctcctcgcagccgcggggcccgctgccgtgggtcgtctccggccgcagcgaggcggccgtccgagcgcaggccgagcgcctggcggcccacctgaccgcgcgcccgcacctggcaccggccgacgtggccaccgcgctggccaccacgcgggcggccttcgaccaccgggccgccgtcgtcggccgggaccgtgaggaactgctcgccggcctcgcggccctggccaccggaacccgcgcgcccggcctggtcaccggccggaccccgccgtccggcggcaaggccgccttcctcttcaccggacagggcagccagcagcccggcatgggccgcgaactggcggctcacagcaccgtgttcgccgacgccctggacgaggtctgcgcccagctcgaccggcacctcgaccggccgctgcgcgaggtgctgttcgccgcggacggcacgcccgaggccgccctgctcgacacgacggcctacacccagcccgcgctgttcgccgtcgaggtcgcgctgctgcggctgctggaggactggggcttgcggcccggcatggtcgcgggccactcggtcggcgaactgaccgccgcctacgccgccggggtctggtcgctcgccgacgcctgcgccctggtcgccgcccgcggccggctgacccaggcactgcccgcgggcggcgccatggtcgccgtgcaggcgaccgaggacgaggtgcgcgcccaactcgccgacggccgccccggcgtggacatcgccgccgtcaacggaccggaagcggtggtgctgtccggcgacgaggccgccgtcacggacctggcgcgcgagtgggccgcccgcggccgggagaccaggaggctgcgggtcagccacgccttccactccgcccacctggacgccatgaccgaggcgttcgccgaggtcgcacgaggggtgtcctacagcgcgccgtccctcccggtggtctccacgctcaccggggcccccgtcaccgacgagctccgcaggccggaacactgggtgcggcacgtccgggagacggtgcgcttccacgacgcggtccgcgccctgcgcgaccgcggggccaccgcgttcctggaggtcgggcccggcggcgtgctgacggccgcggcacgccgatgcctgcccgacgccgcccccgagacgttcgtccccgtgctgcggcgccgcaggcccgaacccgagtccgtgctgacggccgtcgcgcaggcccacacgatcggcctctcgccggcgtgggaccgcctgctgcccaaggcccggacgcgcgtggacctgcccacgtacgccttccagcgcggccactactggctggcgggcatggccggagcgggcaccgcgcggccggtgcggccggaagtgcaggagcccaccgccccctccggtacgccgccgctgtcgcgacggctggccgacgcgtcggaggaggagcgcggccacctgctgctgacgctggtacgcgagcagtcggccaccgtgatgggcggcgtcgaccccgcgcaggtcgaacccgaccgccccttcctggagctcggcttcgactccctgatgggcgtcgagctgcgcaccgcgctcgccgccgactgcgcactgcccctgccgcccggcctgatcttcgaccaccccacgcccgccgccctggccgccttcctcggcgagcagctcgcggcggcggcctccggcacccccacggcggcggcaccctcgccgtactccctggaggcgctgtaccgcaacgccaacaccctcgaccggcccgaggacgcgctcgccctcaccaaggccgcctcccggctgcgcccggtcttcgccagcgtggccgaggcggggcaggacccggtcacggtggagctggcacaggccaccggccttccgggcctgatctgctgcccggcacccgtgccgctgtacggggcacagcagtacagccggctcgcagccgccttccgcggcacgcgcggagtctcggccctgctcgcccccggcttctccccgggcgaactgctgcccgccgacttcgaggtgatgcaggacttcctcgccgagggggtccggcggcagaccgacggcgcgcccttcgtcctcctgggccactcctccgggggctggttcgcctacagcctggcggcccacctggcgcgcaccgggccgcgcccggaggccgtcgtgctgctggacacctatcagctgcacgacccggcgctgcaccgcatgcagcgcgaactcgcccagggcgtcctggaccgcgaggaggacttcggggcgatgacggacgtacggctgagtgccatgggcaaatacttcgacttcttcaccgactgggtggccgaggacgccggtgtcccgacgctgctgctgcgggcctccgagcctctgggcgaggtcgtcgagggccaggagtggcgctccacctggccgttcgacagcacggtcctcgacacggaaggcgaccacttcgccatggtcaacgaccacgcgccgcggacggcccaggccgtgaacggctggctgtcgggcctcaccggcggaaggggctgagcgccggtggagacacgcaacgccgaacggccgtggatacgcagcttccaccccgctccccaggcccctgtgcggctgctgtgcctgccgcacgccgggggctccgcgagcgcctacttcgcgctgtcgagggaactggcgccccgggtggaggtgctcgccgtgcagtaccccgggcggcaggaccggcgcgacgagccgctgctggactcgatcgaggccctgcgcgacggggtcgccgaggccctgacgccctggctggaccggccggtcgccctcttcggccacagcatgggcgccgtggtggcctacgagctggcgcggctgctgtgccaggacgcgggcgtgccgctcacccacctcttcgtctccggacgccggggatccgaccgaagtctccgtccttgccgccgtgttccggaattcaccgtgacaccgccgcgcggctcttcttccgaagtcctccagatccggcacgagtttgtatccgaacggggttctgcgtgcgaaatactctcttcgaattgggtgacatacccccgatcggcaccgtacccgagcagatgtacgcctcggtgatccgacgggagcgctacggacagccccaccaggcgttccgcagcgaggtcgtggacgtgccgaaggtggggcccggtcaggcgctggtcctcgtgatggccgcgggcatcaactacaacaacgtctgggcctccctggggcagccggtcgacgtgatctccgcgcggcagaagcagggccacagcgaggacttccacatcggcgggtccgagggctccggcgtggtgtgggcggtgggggagggcgtcacccaggtcgcggtgggcgacgaagtgatcctctccggctgccagtggacggagacggccgccgacatccggctcggcgccgaccccatgacctccggctcgcagtcggtgtggggatacgagggcaactacggctccttcgcccagttcgccctcgtcgacgactatcagtgccaccccaagccgcccggcctgacctgggaggaagccgcctgcttcctgctcaccggggccaccgcctaccgccagctgtgcggctggcagccgcacgacgtgcggccgggcgacccggtcctcatctggggcggggccggcgggctcggctccatggccatccagatcacccgggcgcggggcggcatccccgtcgccgtggtctccgacgaggagcgggcccgctactgccgggagctcggcgcccagggcaccatcaaccgcctggacttcgaccactggggacggctgcccgacatcggcgaccacgaggcgatgggccgctggaccgagggtgtacgggccttcggccggcgcttctgggaggtgctgggcgagcgcaggtccccgcgcatcgtcctggagcacagcggccaggccaccatccccacctcgatgtacctgtgcgacaacgcgggcatggtcgtcatctgcggcggcaccaccggctacaacgccgacatcgacctgcgcttcctgtggatgcgtcagaagcgcttgcagggctcgcacttcgccaacctgcggcagtgccgcgacgtcatccacatggtcgcgaacggccagctcgacccgtgcctgtcgtggaccggcggcttcgacgacatcggcaaggcacaccagctgatgcacgacaaccagcacccccagggcaaccaggccgtcctggtcaacgcgccgcggaccggcctgaccaccttcgcctgaaccaccgccccggtgttccgacgtcttccccccacacttaccgaccaaggagagatcaccatggacaagctcgacatcctctggagcgagcgcgagatccgtgccgtgctgcagcgctactgccgcgggctcgaccgcctcgacgaggaactggtcaagtccgcctaccacgaggacgcgcacgacgaccgcggcgtcatccgcggcaacgcacacgacttcgtcaagcagatcgtcccgctcctgcgcgacgcctacaccggcaccctgcacaccctgcacggcagcacgatcgagatcgacggggatgccgcgggcgtggagtcctactgcaccgcctaccactaccgcgagagcgacggcatcaagcgggtggagcagttcgccgggcgctacgtcgaccgcttcgagcggcgcgacggcgtctggaagatcgcccgccggctcgtgctgaacgacttcagcctcgcccaggaggtgccgctcgaccccgccgaggcccaggccggcttcaacccctcccaccgcgacctcaccgacgccagctaccaggtgctgccgctgcgcggcccggacgcccccaccctctgagccgtccggccgccccaactcgccccacctcaccaggagtcaccaccgtgtccgacaccgagcagcacgcgcccacgctgccgcggcagcgcacctgccccttctcgccgccgcccgagctcgaggagctgcggcgcaccgatcccatcagcaggatgcggttcgccgacgactccccgggatggctgctgacccgccacgccgacgtccgcgccgcgctggccgaccccggcgtcagctcgcaccccggcaaggcaccccagccctggcgcaacctcgcccccgagatgcgcgccgagcactacctgccgggcttcctgatcttcatggacccgccggaccacacccgctaccgccgcctgctcaccaagtggttcaccatgcgggccatccgcaagctcgaacccaggatcgagcagatcgtcaccgagaccctcgacgccatggaggcccagggcggcaccgtcgacctggtgcagtccttcgcgctgccgatcccgctgctggtcatctgcgagctgatgggcatccgctacgaggagcgcgaggagttcatggacatggtcctgcgactccaggccctggacgccacgcccgaggaactcggggccctcggcgccaggatgaacgagttcatgatgaagctcgccgccgccaagcgcgcgaaccccggcgacgacctgctcagccacctcgcccacgaccccgacgccgacccggcgctcacggatctggagatcgccggcatcggcgtgctgatgctcatcgcggggcacgagacctcggccaacatgctgggcgtcggcacctacaccctgctggagaacgccgaccagtgggccctgctccgtgacgacatcagcctgatcgaccgggccgtcgaggagctgctgcgccaccagaccatcgtccagcagggcctgccgcgcggcgtcacccgggacatggagatcgccgggcaccaggtgaagaccggggagtccctgctggcctcgctgcccgccgccaaccgcgaccccgccgtcttccccgaccccgaccgcctcgacatcacgcgcgagcacaacccgcacctcgccttcggccacggcatccacctctgcctgggcatggagctcgcccgggtggagatgcgccaggcgtggcgcggcctcgtcacgcgcttccccggcctgcgcatggccgccgcgcccgaggacatccgctggcgcgacgaccagatcgtctacggcgtgtacaacctcccggtgacctgggacgaggccaagtgaccggccccgaggccgcggtgcgcgggtgccccttcggcgccggcgaggcgcccgcgtaccccttccacgcccccgaccggctggagcccgacccgtactgggagccgctgcgccgcgagcggccgctgcaacgcgtcacgctgccgtacggcggcgaggcgtggctcgccacccgctatcaggacgtgcgcgcggtcttcgccgaccgcaggttctcccggcagctcgccgtcgcgcccggcgctccgcgcttcctcccgcaccagccgccgccggacgccgtcctgagcgtcgagggccccgaccacgcgcggctgcgccggctggtcgggaaggtcttcacgccgcgccgcgtggaggacatgcgtccgctcatccagcgcaccgccgacggactcctcgacgcgatggaggagatggggccgcccgcggacctggtcgaggacttctccctgcccttcgccgtgtccatgatctgcgagctgctcggcgtgccgcccgaggaccgcaagcggttctgcgtctggtcggacgcgctgctgacgaccaccgcgcacacccccgcccaggtgcgcgactacatgatgcagatgcacgactacctcggcgggctcgtcgcgcagcgccgggtgcggcccaccgcggacctgatcggctccctcgtgaccgcgcgcgacgaggaggacaagctcaccgagggcgagctggtgcggctggccgaggccatcctcatcgccggctacgagacctcggcgagccagatccccaacttcctctacgtcctcttccgccacccgcagctgctggagcggatcaggaacgaccacgacctcatccccgacgccgtcgaggaactgctgcgcttcgtgcccatcggcaccgtggacggctttccccgtacggccaccgaggacgtcgagctcgggggagtcctggtcagggccggggagacggtcgtgccgtcgatgggcgccgccaaccgcgaccccgagctgttcacggaccccgacgagctggacctcgcgcggcggccgaatccgcacctgggcttcggcgcgggaccgcaccactgcctgggcgcccaactggcccgggtggagctccagatcacgctcacgacgctgttccgcagatacccccgcctgcggctggccgtgccggaggagagcctctcgtggaaggaggggctgatggtccgcggcatgcacaccatgccggtcacctggtgaggacaccggcgtcctcctgaccttcccggcgttctcacgccgtcccggcagccttccttccgacacgagcgcacagagggtgaagcgaccgcaatgagcaccatcgacgaatgggaacacagcacgaaggaggcgggcatggaccccgcggccctcagacgcctgaccgatgtggtgcgggcgaggggcggcgcggcgcagctgtgcgtcatgcggcggggcaccgtggtcctggaccgctcgttcggctgctcctccgactccctcttcctcgtctacgcggccaccaagcccgtcgccgccctcgccgtgcacgcgctcgccgagcggggcctgatcgggctggaccggccggtggccgaatactggccgcagttcgcccggcacggcaagggtgacgtgaccgtccgtcatgtcctccagcaccgggccggggtgccggtcggccggggcatcgtgcgcacgatgcgcaccgccggcgactgggagcgctccgtgcgcgaccttgagcagtcccggcccaagtggcccggcggcgaggtcgccgcctaccacttcatgagtttcggattcattctcggcgaactggtgcagcgcgtcaccgggcggtcgttccgagatttcgtgacttccgagctcttcgccccacttgggctgaatgatttgcacatgggattgcccggcagtgcctggccccggcatgtgcccgcgcgggccgcccacccctccgaatggcccaatcagtggatgagcaaccgccgcggctaccgccaggccgtcattccgtccgccggtctttccggaaccgccgcacaaatggcccgcttttaccagatgcttatggagggcggctcgctcgacggcatccgcgtgctgcggcccgaaactgtggaggaagccagaaaaccgtccaatgacggcggaatcgacgcttccctcaagcgtccggtccgctggtcccacggattcatgctcggtggtccgggcccggacccgcgggggctgtccaatgtgctgggccgcacgagcgacccgagcgccttcgggcacgcgggcaccacgtccagcgtcgtgtgggccgaccccacgcgcgagctggtcctcgcctacctctccaacatccagcccgagttcggagcgggtatcgagcggctccgcgaggtcagtgacctcgcgctcggtgcctgcgaggcaggctgacccgagccgtgccgccacggcccggcgcccgcccgatccgatcgggtccggtgggggccggccgggtccgggcggggacgcacttcccccggcgtccccgcccgggccccggtgcgaaccgggcgcaaaggcggccgatcgcccggcgcggccggatgcccccgaacggtgtgaaacgttcttatcagcctctgaccagcaccgagtgatctactgcacagcccgaggccgcgattccggcagtatcttgatcttgacggggcaccaatgcgagcgggctattcgccgcggttttccctgacgtcggatgcagatgacaccggaggagggccagtgctgaatctgcccaaaggaatggagcgcgcgcatccgcattctccgccacaggtgggaatactcggacccttggaagtccgctcggccggaggtgccggaacgggagccgcggtaagcggtattcgcgtacgcacattgcttgccgcgttgactgcccgcctggggcaggcgatgtcgaccgagcgcatcctcaaagaggtctgggccgacaacccgcccgcgaccgatcgcaaggcggtggccgtcgccgtcctgcggctgcggcgggtcctcggcgacaacgaaggacggtggctgctcacccgcccctccggttacgtcctggacatccccccggaccacctcgacgccgtacgcgcggagaccctggtgcgggaaggccgggccgccctggccgccggcgacccacgcgtcgcggcccgccacctcacgcgcgccctcgaccagtggcggggcgagccctacgcggacgccaacgccatctcgaccgtgtcccagcgcatcacggagctggagaacctcaggtccgaggccgtccaggcgcacatcgacgccaggctcgaactgggtcaccaccaggaactggtcggcgaactccgctcgctgaccgccgcgaaccccctgcacgagccgcactggctgcagctgatgctcgccctctaccgctccggcaagcaggccgaggctctcgccgcctatatgcagctgcggcaggcgctggccgagaacctgggcatcgacccgggtcgtcagctccaggaactgcacctgcggatcctgcgcgccgacgcgggcctgctgacggggtccgggccggcggcaccggccgagccactgctcgtacggcagtcctgagggctcacggccacccgaagaacgcgcggtagcacggaacctgctgctccagcatatggatgccgtggtgcacacggcgcccggcggtggcggccgcgctcagcagcgccgtctcgtgcggcttcatgacgacgtcgaccaccacggcatccggtcgcaccctcgcggggtcgaagggcagcgggtcctcggaacgcatgcccagaggcgtcgcgttgacggcgaaatcggccgcctccagatcgccgggccccagcgcccggatcccgtccggccggcgggacccgagccgcagcagcagcgcgtcgagctgggcgcggtcggtgtcgtgcacggacacccgcgcggcgtcggccatcagcagcgccgtggcgatcgcgctgcccgcccctccggcgccgaccagtgccacatgcctgtcgcgcaccgtgtgcccggccgcctgaagaccctggacgaacccgagcccgtcgaagttctcggcgtaccagcggccgtcgggttcgcgccgcatcgcgttggccgtcccgatgagggcggccgccggcccgagcccgtccgcgagcccgcacagggccgccttgtgcggcacggtgaccagcagaccgtccagattgccgatccgcttgagcccctcgaccacctcggcgagatcccgcgcccggacgtgcaccggcaccaccacggcgtccagaccgctttcgctcagcagggggttgagcagaccgggcgccttgacctgggcgacgggatcacccagcaccgcgtacagccgcgtggcgcccgagacaccggccgccggcccgaggaattccatcagccgatcctctctgtacccccgacggatgttgccctacggtgctggagatgctccacagctttgccgtgaccgccggtcggcacaaccctgcgtgcccctgacgcgccaggccctccaggtagttgctcccggcggatcccgacagctcccgaccggtcccgacggagggaagaagccatcagatacctgggaatcgacgtcggaggcacgaaggtcgccctgcgggtgacgggggacaccgacggtgcgggcggcggcgacgtgacgttccgctggcccgccgccggcgacgtcaccgcggatctggacctgctcgccgcgcgggtccgcggtcttctgggacaccgcgaggaccccctcgccggggtcggcgtggccatgcccgcgatctgcgacgcggccgggacggtccgcacgtggccgggacggccgagctgggcgggcctgaacctgacggccgccttcgggcagttgctgcccggcaccccggtcgcctgcgccgacgacggtgacctggccgcgctggcggagtcccgcgccgccggctgccggcatctgctgtacgtgggggtcggcacgggcatcggcggcggcatcgtccatgagggccgcgcctggccgggccccggacgcggctcgtgcgaggtcggccatgtcgtcgtcgaccgctcgggcccacgctgcgactgcgggcgcgccggctgcgtccaggcggtcgcgtcgggaccggcgaccctccggcgggccgccgaacggcgcggccgggagaccggcttcgacgaactggcctccggggcgcgcttgcacgccccgtgggcggaagcggccgtcgacgagagcgccgcggccctggccaccgccgtgaccggcatctgcgagctggcccaccccgaactcgtcctcgtcggcggcgggttcgcggcgggcgtgccgggatacgtggcctcggtggcggcgcacgtcgagcggctgacccgcccgggaacggatcccgtgcgggtgcgcccggcggtgctcggcgggcggtcctccctgcacggcgcactgctgctcgcgcgggaggcacacgggcggggaaaccggccgccggagagtgaccgtgtttcttccgatgtttcttccgatgtttctttcgggggagtgacagacagggccgttggccggtccgactgagcacaatcacaggtgatttcgcccaggttcaccacgcctcgtgtgctcggggtcggcagaaggagtcagagtcatgctcgacaggcggagcgtcattcgcgtcggcgccggggtggcggcggccgccgccgtggccggtacggccgccaccggtgcggcggccgtggggctgccgggtgtacggggacgcgcggcgtcgcgcggggtcgactgggcctccttacgccgtcatctgtcgggcgagctcgtcctgccggcggacaccggatacgagcgggccaggaagctctacagcggccagttcgacggcatccgcccgcaggccgtcgcctactgccggaccgaggaggacgtgcggacgaccctcgcgttcgcccaggaccacgcgctgcccctcaccccgcgcagtggcgggcacagcttcggcggctactccacgaccgacggaatcgtcctggacgtctccggcttccacgcggtgagcctcacccggaacaccgtcgtcatgggcgcgggcacccagcaggtggacgccctcaccgccctgtcgccgcgcggtgtcgccgtggcgagcggcaactgcgcgggcgtctgtcccggcggcttcgtccagggcggcggactgggctggcagagccgcaagttcggcatggcgtgcgaccggctcgtctccgcccgggtcgtgctcgccgacggccgcgccgtgaccgcctccgccaccgaacaccccgaccttttctgggcgatgcgcggcggaggcggcggcaacttcggcgtcgtcaccggcttcgagctgcgccccaccgacgtcccctccgtcgtcagctacaacctcacctggccgtgggagtcggcgcggcgcgtcatcgaggcgtggcagcactggatcatcgacggcccccgcgacctcggtgccgcgatggccgtgcagtggcccgacgccgggaccggcacgccggtcgtggtcgtcaccggcgcctggctgggcgcggccgacgcgctcacccccgtgctggactccctggtggcctccgtgggcagcgcgcccgccacccgctcggccaaggcgctctcccagcacgacgcgatgatggcgcagtacggctgcgccgacctcacgcccgagcagtgccacacggtcggctactcgcccgaggccgcgctgccccggcagaacttctccatggaccgcaaccggctcttctcccgggccatcgggcaaggaggcgtcgagcggatcctggaggcgttcgccgccgacccgcgcgccggacagttccgcttcctgagcttcttcgccctcggcggcgccgccaaccgccccgaccgcaccaccaccgcctacgttcaccgcgacaccgagttctacctcggtttctcgatcgggctgaacgacccggagtacacggcggaggacgagaggctcggccgcgcctgggccgcgcgaggactgcgcacgctcgatccccactccaacggcgagagctaccagaacttcatcgacccggagctcgacgactggaagtcggcctactacgccgagaactacgtgcgcctggccgccgtcaaggcggcctacgacccgcaccggctcttctccttcgcgcaggccgtctgacctctcccgaaagacccctgccggcctgctcccctccgcggctcctgtgggcactggtgcgcccgcgcacttctgtgtgattgagtgaagtccgggcgtgcagagctcagttgccgtggagggggcgccagttgcgagcatcagcggtggagagggtggagctgatccgctggccggtggagtccgagcggcgggagcgctgccgcgaccggggcgtcatgcggatcctggtgctggaggcgggggccgaggcacccttgtgcgtggaccccaaggaggactgggtccgcgctcccgtcagcaccgacgacctgcgggcccgcgtcgaggccctgcgccttcggggagccgccgccgagtcccggcccgaggtcgacccgaacggagtgctgcgtttccggtggcgctccgccctgctctcgcccaccgaggcccggctcgtcgcccggctcgccgagtcctatgccgaggtcgtcgcccgcgacgacctgctccgcccgcccccgggccgtaccgtgccgagccgtaacgcgctcgacctccacatcatgcggatccgacggcgcctcgccgcgctgggcctgagggtgcgcaccgtccgggggcgtggctacgtcctggagagcgcggaaggagtctgaccgacgggcgtggccgcgcaccgcaccgaccgcccctacgagcgaggagcccgaagtgcagcagcctcatcacagccgcgtcgacgtggaactgggcgagaggtcctaccccgtccacgtcggaccgggggtccgccacctcctgcccggcatcgtcgcctccctcggcgcgcaccgcgccgccgtcgtgaccgcacggccccccgacctggtgcccgatcccggcgtgcccgcgctgatcgtgcgggcacgtgacggcgagcggcacaagacgctcgccaccgtcgaggacctgtgccgcaagttcaccaccttcggcatcacgcgccacgacgtcgtcgtctcctgcggaggaggctcgacgaccgacaccgtcggcctggcggcggcgctgcaccaccgtggggtgccggtggtgcacctgccgaccaccctcctggcccaggtggacgcgagcgtcggcggcaagacggcggtcaacctgcccgagggcaagaacctcgtcggcgcctactggcagcccaaggccgtgctgtgcgacaccacgtatctccagacgctgcccgccgaggagtgggtcaacggctacggcgagatagcgcgctgccacttcatcggtgccggcgacctccgcggcctcgccgtccacgaccaggtcaccgcgagcctgcggctgaaggcgtccgtcgtcgcggccgacgagcgggacaccggcctgcggcacatcctcaactacggccatacgctgggccacgcactggagaccgccaccggcttcgggctgcggcacggactcggcgtggcgatcgggacggtcttcgcgggccggctcgcggaggcgctgggccgcatcggcgccgaccgcgcgcgggagcacaccgaggtcgtccgccactacggacttcccgacagcctcccgggaaacaccgacatcaccgagctcgtcgcgctgatgaggcacgacaagaaggccacgtcgggactgaccttcgtgctcgacgggccttccggcgtggagctggtgtccgggatcccggaggacgtcgtcctgcgtacgctcgcggcgatgccgcgaggaacggcctgaccgagtgttccgtcttccgaggggaagtgaccgtttcgtgtcggcagagctgtcagaaccgctgaagaaggccctggactccctggtgttcggcgtcgtggcgacgaccgaccccgacggccgcccgcaccagtcggtggtgtgggtccggcgcgagggctccgacgtgctgttctcgatcacgcgcggcagccgcaaggagaggaacatcctgcgcgacccgcgtgtgagcgtgctgatcagcccggcggactcgccgtacacctacgccgcgatccggggcaccgcgcacttcgaggacgtgccggacccgggcgcgtacctcgacacgttctccataaagtaccacggcgtgccctaccgggagtcgttccccgagccgccggaggtgagcaccattctcgccgtccggctcgttccgacgtcggtctacgagcagtggtgagggcgtaggcgtcccgaagccccggcagcgtcccgaatgccgctgccggggcttcccgtgggagccctacgccggtttccgcgcggtgaccaccgagtagccgacctcctccaccgagcccatgcggtcgatgccgtcgaccatgcggtggaacgcctcgtcgtccatgtgggagccgagctcgtccctggccgcacgcatcttcgccgccaccgcctcgtaggagggccgcacctcgtccccgatgtcgaggaactccaccacctccagccccaccgaccgcatgcagtcctcgtacgcctcgcgggtgaggacggggccctgctggaagttgtcgttggcggtgtcg

SEQ ID NO: 97 - DNA, 1167 bp, Streptomyces lavendulae
atgacacctacgtccggtgatgacgtcctgtcctttccctcatggccgcaacacggcgcggaggagcgcgccggactcctgcgggccctggaccagaaggggtggtggcgcgacgcggggcaggaggtcgatctcttcgagcgggagttcgccgaccaccacggcgccccgcacgcgatcgccacgacgaacggcacccacgccctggaactcgccctgggggtcatggggatcggccccggtgacgaggtcatcgtccccgcgttcaccttcatctcgtcgtcgctggccgtgcagcgcatgggcgcggtgccggtgccggcggacgtacggcccgacacctactgcctcgatgccgacgcggcggcggcgctggtgacgccacgcaccaaagcgatcatgccggtccacatggcgggccagttcgccgacatggacgccctggagaagctctccgtcgcgacgggcgtgccggtcctccaggacgccgcgcacgcccacggcgcgcagtggcagggccgccgggtcggggagctcggctcgatcgccgccttcagcttccagaacggcaagctgatgaccgccggcgagggcggcgccctgctcctgccggacgacgagtccttccacgaggcgttcctccagcactgctgcggccgcccgcccggggaccgcgtctaccgccatctgacgcagggctccaactaccgcatgaacgagttctccgcgagcgtcctgcgtgctcaactgaagcgcttgaaggatcagttgcgcatcagggaggagcgctgggcccagctgcgtacggcactggccgccatcgacggcgtggtgccgcaggggcgcgacgagcgcggcgacctccactcccactacatggccatggtccggctgcccggcatctcggcccggcgccgcctcgcgctggtggacgcgctggtcgagcggggagtgcccgcgttcgtcggcttcccgccggtctaccgcaccgagggtttcgcgcgcggcccggcgccggcggacgccgaggagctggccaagagctgtcccgtggcggaggagatcggcagcgactgcctctggctgcaccatcgcgtcctgctcgccgacgtgaccacgctggaccggctggcggaggtcttctccggcctcgtcggcgcgctctga

SEQ ID NO: 98 - DNA, 819 bp, Streptomyces lavendulae
gtggtcgtcgtcgacgacaacgacgggggcgacgccggtgatcaactgatcgccgtgacaggcgagatgagcggccttctcccgctgcgcgtggtgcggggaccgctgcgggggcgggccgccgcccggaacgccggggcggccgcggccctcgcgccccggctggtcttcctcgacgacgacgtcctggtggggcccggcttcctcgccgcacacgccgcggccgcggaaccggacgccttcacccacggccggctgcgcgaactccccaccgcggcgcggttcctcgccgctgtcgagaaggccgccccgaccgaggtccgccgcgcccgcgccggactcgaacccgctgccccggccgcctccgagcggcgccaaccgcaccggcggctcgtcgccaacgccctggagcgggccgtggaggccatggccggcggctccctgccggacgtcgccccctggctcggcttcatcggcgcgaacaccgccctcgacaaggccgcatgggagcataccggcggattcgacgaggagttcgggctcacctgggggtgcgaggacctggagttcggcttccgcctgcacgccgccgggctgcgcaggaccctcgcccccgacgccctcggtgtgcacctcagccacgcccgccccggccgctgggagcagcaccaccgcaacctcacgcacttctccgccggccacccgcacccgtcggtacgcgccttggaggccctgctcgggcccgacggcacgccggaggcgtatgtgcgcgccgtcctggccgaagaggccgcaccggcacgggacgcggcgcgatga

SEQ ID NO: 99 - DNA, 783 bp, Streptomyces lavendulae
atgagcggcacaccggccaccgcgccgtacggtcccgtggtgctctccccgcacgcggacgacgccgtgtggtccctgggcgggcggctggcgcgctgggccgccgagggcccgcggccgaccgtcgtcacggtcttcgccgggcccgcggccgggaagcccgagtcgtggcggagcgccgccgatcccgcggtgcgccgggccgaggaccgggcggcatgtgccgaactgggcgtgcgccacgtgccgctgggcttcaccgacgcggcactgcgtacggcctcgggcgcctatctctacgcttccccgcgccggctcttcggcccctggcacccggccgacctcccgctgctggaggaggtgcgggcggctctgctgccgctgtgcgcgggggcgtcgagcgtccacgttcccctggcggcgggccggcacgtcgaccaccgcctggtccgcggcgcggtggagcccctgtcccccgcccgtaccgtcttctacgaggacttcccctaccggctgcgcgaacgtgaccacacgaacctgcggccgcgcacggaacggctgccgtccgaggcggtggaccgctggctgaccgccgccggtcactactccagccaggcgagcgcccacttcggcggtgcggccgccctgcgcgaggccctgttcgcccgcgcccgcgcacacggcgggcccggccggcccggccacgccgaccgccactgggtgcccgtcggccaggacgaccggggcgaggcccggccggcacccgtggaaagggggccgtga

SEQ ID NO: 100 - protein, 383 aa, Streptomyces lavendulae
MSRSTHPPTATPDAGTRRRLPLIGNDLVINEDSCNLSCTYCLTGQSNLKEGHSLQLIFEPPRRDSYAKDSGLGQRMDKVADRIRDRFGLPLLKVTGGEIFLVRGIMDFLEQEARKYDVLVIQTNGVLVREEHLERFRSWGNVVLQVSLDSHLHHGNSHRVPSGSLHEKVVAAIARILDSGLPVEIYSVLNDRSVTEVCAFAEWLSGFSRPPVYFPFPVRGPDSEDFKVRPGQFGHIQELVDRYDEFARVLPPRPYFDRLTSFYREGRRTFRCHLPRLVVSSFSDGVVTPCPNIWFSDMGNALEDDWSEMLDTVGTSGLYRALLAPKPRLKACHGCFTPWDTLSMYFEDEITLDELCAAPTYSPPRIRQMLSDAKADYLQGGHD

SEQ ID NO: 101 - protein, 707 aa, Streptomyces lavendulae
MALRAPNSPRWVVAFLSLLASGARPLLLEPDTPGPETARLLRAAGGGRSLVVPGTGDGLRLTLTGSPGEPSGAPPAVLLPTSGSTGASKLVARSEESLLAEGRRYRDGVGLTGEDTLLLPVPLSHAYALGWLFGGLLTGAALRPVPPTALGRIAAELSGGATVVALVPSVARLLATRRLRGAAAGRAPAAPGLRLAMVGAGPVDEQLDRAFTEAFGTGLARNYGSTETGAVLAGPAGLEPLCAGAPLPGVECELTGPEGVVPPAGTPGLLSVRVDGRPYAMGDLAVAVPGGLRILGREDRAIRRGGRWVSPLEIEEVLRGHPDVVNVRVGARRGRHRGEDGIVAEVSAAGPGLTPEALREHARRELAPHKVPDEFVLRESLPVNAAGKVRAASVYRLTRSAAEAARAYKASEVLFALHDLGALEALAQGAGTALLAGELGCDADALEWLLRTATALGVLTTGAQEPGDRVRAGELAAFVALEEHLSRGLVTREELVAVARSGTARRPFEERPPESLGPLVALYQGAMDGPGARARAALGLRLLRPGPGARVVEVTAGPGRYLERLLASDPGASGHLVTVGRLSGPLSSAVAAAVEEGRVTVGTELPVGYADFCVVANAVHGPGPGSALGALLGSLRPGGRLLVDDVFLPASGPGSELALDWLTHGGTAWPATGELIAGLLQEGAEVARHVPLDASPCHLIIAKEAGS

SEQ ID NO: 102 - protein, 257 aa, Streptomyces lavendulae
MSTVTDRATERLGQSGRVVVVSGASGQIGGACALELAALGATVVAGYHSGEQAIRKLREQVEGQGGTLVPVAADLSEPEGADALVAAAVERFGRVDGCVAAAGLRTRRLAMATDARSLEKLLRVNLAGSVGLAKACLKPMMRARYGRIVLFGSRAGTSGLPGHSAYAATKGALQPWAASVAGEVGKHGITVNVVAPGAIRAEVMDFSEAERDLVLQFIGAGRLGEPEEVAAAVSFLLSPSASYVNGNTLVVDGGARF

SEQ ID NO: 103 - protein, 404 aa, Streptomyces lavendulae
MTPPTTAREPLRMAVLGAGWVSRKVWLPLLAEHPAFRVDFLVDDDPVAARSALPEGARTRVLSRPEELAPRSVDAAIIALPNHLHLPVAKALLERDVPVFVEKPVCRTLFEAQALALDHQARGDSIGDITLYAWSAARHRTDVCRLAELLPSLGTVRSVGLSWIRATGIPQRTGWFVDRRLAGGGALLDLGWHLLDVGLHLLGWPRVVRAASTMSADWMSRGEATADWSRRSSGTARPGPGETVEDTARGFLVTDTDVGISLETRWASHQALDVTTITVEGTEGVATLRGTFGFSPHRLQKSSLVVLRQGVEETVALPDEPVGVEYRRQVDELARRLGGSADGQGPVSGLGEGSMAEVTILASCIDHIYSAAGVDPPSPLHRPQSDAAPSTSSCPRVLPTRGSQ

SEQ ID NO: 104 - protein, 382 aa, Streptomyces lavendulae
MKFAYFSHVWGRPGITPGERYEELWREVEDADRLGFDYAFSVEHHCTPHESWMPSPAVFCTGAALRTERIRVGPMGWVPPLRHPLHLVEEVATLDQLLGGRLEVGLASGVSRDPFLPFDADFDNRHLLTREALELLRAAFAARGAFDFDGPAHRLRDIALSFPPVQRPHPPMWVPTTNRNTLRYLSEAGAHTSSTMIVPRASMALVYRHYLDWWRGHGHASDPRIGYWTLVHVARTDAEAEERAAAHITETFTKTLRYGSVSRSRDQHAPPSRLSTTDILAGSGDLRFLLENNLVFVGSPATVADRIRAASLEGHFDTLLGEFTFGELADRHRIESMELFAHEVAPALRAFSPYAPRPQEPAYTASDEQQVAARLQALGYID

SEQ ID NO: 105 - protein, 290 aa, Streptomyces lavendulae
MERLKLVPDEHRRFTVDEQSARRLHRIGPELLSALCEAGVPFVGSGAGRLFDGYDLGNAALHLGLSSVQRRAIRSWAGSLRTASAAESPRWRVDVTASCPVPGHAGPCRYGVLLPGARRPVEAASPRETTLARLYTRSRGHWPDFPPAVLDLLRTLEPVGFFLLPEAIRWDPGFLWSTHMADCGGAAAWLVAEGRRRGLDVRFSFGLLVAKPYSTPHCWAEFLVGGRWVPADPLLLRAMAAWGGLDAAAHPPHSSPGAVYHRLAGRFTKVVSHAGVWAPTSLPTELLPCP

SEQ ID NO: 106 - protein, 235 aa, Streptomyces lavendulae
MPLNPPPASRAAADAPATALPCRFTTVVFDLDGVLIDSFAVMREAFAVAYREVVGPGEPPFEEYRTHQGRYFPDIMRLMGLPGEMEEPFVRESHRLMDRVEVYPDVPQLLAELRADGVGTAIATGKSGSRARAVLEAVGLLPLLDEVVGSDEVPRPKPHPDIVREALRRLDAAPEDAVMVGDAVIDIRSGRAAGTATVGATWGEGAAGQLRAERPDFLLDKPQSLLALVRSGGHA

SEQ ID NO: 107 - protein, 346 aa, Streptomyces lavendulae
MTPATPRWSVVAPQGTNLELAGTGGREGWRLLLETARTVHRHGRGALWLLDRTDTLPRREPEPVWEGWTALAALAGAVPGLDLGLLSSAPPFRNAALIAKRAATLDVVCDGRLTLGFPAREYLPEHHSTGREVPTGLEADEEEAAGHRALGETVEALRALWGGQPVTFTGEHIRLTSAHCVPAPRQQPLPLALRTPAGDAGSGALRPADATVRECAHVQWTGEPAQVAAAVTAFRRRRTELGLDPDGVRHAWAAECRIFDSVLERDRWLSTPHEVLFWSHHPDLLARRSLYGTPEQLTERARRLVAAGVAEFVLWFRDYPATTSLERLFQEVVPQVAPGAAKEAEE

SEQ ID NO: 108 - protein, 520 aa, Streptomyces lavendulae
MLNTLSTAPFLSTAWLAGAARLERPPVGERGTVALRLELTDPPPGEPPAVDVQVDLVAGRLGLAAAAGESPGLRIRLPLEAARALLLGPARDRTGVFERGDVRAEGNFSLLFFIDAALERDASGHVAALRGTPGTTAREAAPPPGTEDAAEAVRRARAALPGTMRELEREVGTSTPGAQIYVSRDGVPLADAGLGLARPGVAMTHRSLPLWYCCAKPLLSVALGRLWEAGAYDPYLPVAHYLPEFGNRGKESITSMELLTHTGPLPTGDDPLHGIVAGPDEERVRRAFEVPVAPRPGGTPGINYSQWWAWFVLARILPVVDGREYRAYVQEEILGPCGMSGTRVHLDREEFAALGGELPLIHVSNPEGGPLPTHWWSTEAATTRCIPGVNTRGPLRDMGRLFEMLLRGGDAPGGRVLAPPTVAALTARHRTGLQDRYGNADWGMGFRLECRQLDPRFTSFGSYASPRSFGHDGLWTAVVFADPDAALVVALHLNGKVEHERHRERIVRLADAVYQDLRLS

SEQ ID NO: 109 - protein, 283 aa, Streptomyces lavendulae
MPHSELSELPMPSPASEEVGALYDRFTALGAASLGENLHFGYWDSPDSQVPLAEATDRLTDMMAERLRIGAGSRVLDLGCGVGTPGVRIARLSGAHVTGISVSHEQVVRANALAEEAGLADRARFQRADAMDLPFEDESFDAVIALESIIHMPDRAQVLAQVGRVLRPGGRLVLTDFFERAPLAPEGRAAVQRYLHDFMMTMVSAEAYPPLLRGAGLWLEEFLDISDQTLEKTFRLLSERINSSKQRLETQFGEEMVNQFDPGDLVGVKEFGYLLLVAQRPGK

SEQ ID NO: 110 - protein, 275 aa, Streptomyces lavendulae
MTETASASDRMVELYNRVTDLMVHAEGGYMHGGYWAGPDVPTTVEEAGDRLTDYVSERLRLAPGERVLDVGSGNGKATLRIAARHGVRATGVSINPYQVGLSRQLAEKEGDEATEFRIGDMLALPFPDGSFDACYAIESICHALERADVFTEIARVLRPGGRVTVTDFVLRRPLSDASRTIVDTANDNFQQGPVLTREAYEDCMRSVGLEVVEFLDIGDEVRPSYEAVAAKMRAARDELGSHMDDEAFHRMVDGIDRMGSVEEVGYSVVTARKPA

SEQ ID NO: 111 - protein, 146 aa, Streptomyces lavendulae
MFRLPRGSDRFVSAELSEPLKKALDSLVFGVVATTDPDGRPHQSVVWVRREGSDVLFSITRGSRKERNILRDPRVSVLISPADSPYTYAAIRGTAHFEDVPDPGAYLDTFSIKYHGVPYRESFPEPPEVSTILAVRLVPTSVYEQW

SEQ ID NO: 112 - protein, 343 aa, Streptomyces lavendulae
MQQPHHSRVDVELGERSYPVHVGPGVRHLLPGIVASLGAHRAAVVTARPPDLVPDPGVPALIVRARDGERHKTLATVEDLCRKFTTFGITRHDVVVSCGGGSTTDTVGLAAALHHRGVPVVHLPTTLLAQVDASVGGKTAVNLPEGKNLVGAYWQPKAVLCDTTYLQTLPAEEWVNGYGEIARCHFIGAGDLRGLAVHDQVTASLRLKASVVAADERDTGLRHILNYGHTLGHALETATGFGLRHGLGVAIGTVFAGRLAEALGRIGADRAREHTEVVRHYGLPDSLPGNTDITELVALMRHDKKATSGLTFVLDGPSGVELVSGIPEDVVLRTLAAMPRGTA

SEQ ID NO: 113 - protein, 164 aa, Streptomyces lavendulae
MERVELIRWPVESERRERCRDRGVMRILVLEAGAEAPLCVDPKEDWVRAPVSTDDLRARVEALRLRGAAAESRPEVDPNGVLRFRWRSALLSPTEARLVARLAESYAEVVARDDLLRPPPGRTVPSRNALDLHIMRIRRRLAALGLRVRTVRGRGYVLESAEGV

SEQ ID NO: 114 - protein, 514 aa, Streptomyces lavendulae
MLDRRSVIRVGAGVAAAAAVAGTAATGAAAVGLPGVRGRAASRGVDWASLRRHLSGELVLPADTGYERARKLYSGQFDGIRPQAVAYCRTEEDVRTTLAFAQDHALPLTPRSGGHSFGGYSTTDGIVLDVSGFHAVSLTRNTVVMGAGTQQVDALTALSPRGVAVASGNCAGVCPGGFVQGGGLGWQSRKFGMACDRLVSARVVLADGRAVTASATEHPDLFWAMRGGGGGNFGVVTGFELRPTDVPSVVSYNLTWPWESARRVIEAWQHWIIDGPRDLGAAMAVQWPDAGTGTPVVVVTGAWLGAADALTPVLDSLVASVGSAPATRSAKALSQHDAMMAQYGCADLTPEQCHTVGYSPEAALPRQNFSMDRNRLFSRAIGQGGVERILEAFAADPRAGQFRFLSFFALGGAANRPDRTTTAYVHRDTEFYLGFSIGLNDPEYTAEDERLGRAWAARGLRTLDPHSNGESYQNFIDPELDDWKSAYYAENYVRLAAVKAAYDPHRLFSFAQAV

SEQ ID NO: 115 - protein, 315 aa, Streptomyces lavendulae
MTGDTDGAGGGDVTFRWPAAGDVTADLDLLAARVRGLLGHREDPLAGVGVAMPAICDAAGTVRTWPGRPSWAGLNLTAAFGQLLPGTPVACADDGDLAALAESRAAGCRHLLYVGVGTGIGGGIVHEGRAWPGPGRGSCEVGHVVVDRSGPRCDCGRAGCVQAVASGPATLRRAAERRGRETGFDELASGARLHAPWAEAAVDESAAALATAVTGICELAHPELVLVGGGFAAGVPGYVASVAAHVERLTRPGTDPVRVRPAVLGGRSSLHGALLLAREAHGRGNRPPESDRVSSDVSSDVSFGGVTDRAVGRSD

SEQ ID NO: 116 - protein, 514 aa, Streptomyces lavendulae
MPPSPRALVIGIDGGTFDTVDPLIECGLLPHMAKLLRESASAATDCTWPAHTAPGWSTFVSASDPGGHGIYQFYDTQDPAYGARVTRSGDLGRSCAWDWLAAQEYSLGLINIPMSHPPADLPGYQVTWPLERTLKHCRPDSLLRELAAAKAHFQSDLATMFRGDMAYLEEAERNVAARVRSVRHLMSTRPTDVVMVVLTEADRVGHHYWHYGDPGHPGHRPAPEGSGWDVAMPRIYQAIDHAVGELLELVDEDTSVVLVSDHGLGTGRHGLSVHTLLEEAGLLATAPGEEPQDAAASWFAGNGRHVDFRRTSVYMPVPGSYGLNINVRGRQQRGTVAPRDRERVMDEVTGLLSGLTGPEGQQVFRAVRPREEAYPGPHTGRAPDLLLVPRDETVLPVPDLGGDVWRPSAQTGLHRYRGLWAHRSPRVRPGRLPGTVALTDTLPTLLTDLGAAWPSDIHGRPVTAVLDDGVRVPPSDPRVEATGTPATTIPAAASAADAAEDAYTSDRLREMGYL

SEQ ID NO: 117 - protein, 93 aa, Streptomyces lavendulae
METLTTDKIKDRLRKVLVDSLELSLDPSAVPDEGLVEKLGLDSINTIEFLIWVESEFGIEIADEDLSIKLIDSLDLLAGYVSERVNGVTAPAE

SEQ ID NO: 118 - protein, 470 aa, Streptomyces lavendulae
MDRHALVIGLDGMPRTLLTRLAGDGTMPHTAALLAEGHCAELLAPVPEISSTSWATFLTGTNPGRHGIYGFTDLAPGDGYRITFPGVRQLREPPLWELAARAGRRTVCLNVPGTYPAPAIDGVLVSGFVAPELERAVSPPRLLPLLRGLDYELDVEVGDVAADPAAFLGRAVRALRARTRAMEHLLRQETWDLAVAVLTETDRVHHFLWRAVADPADPLHGDVLAFYRLVDDCVATLVSTLPPGGELFLMSDHGFGPAACQVYLNAWLRESGWLAGLDVCPDLTAVDARSTAFALDPARIHLNRKSRFPGGGLTDAEADEAAHEIARELSALRCDGTRLGPDVDGPLLVRDLYRAQEIYHGPLLGNAPDLVAVPAPGVQLRGGWGGTHTVRNDILTGTHTRDDAVFYRRGAPAPAPGADDGPLDMTDVAPTVLASLGIHPGGLDGAAVLGTTGPASGHGRTDPPLDIREL

SEQ ID NO: 119 - protein, 611 aa, Streptomyces lavendulae
MKHDLGLAPSAPKPGTLDLSLDPRITDPASFRVSFLILLDGDLVMSPEHLGVAYMAGVLRHTGFTAEIREVEHGDDQAAATVEALKEYRPDLVCFTLMSLNLGSCLTLCRMLREELPGTTIACGGPAGTFAGLDVLRNNPWTDVVAVGEGEPTILDLVQRLYLKEPLSACKGICYRDEDGTPRQNPARPLIHNLEDLPFPARDQLRQHGDKLEYVRVSTSRGCVANCAFCSAPHLKNRVQAGKAWRGRGPEQIVDEVAEIVERHQFRTFDFVDSTFEDPDGGRVGKKRVAAIANGILERGLDIYYNVCMRAENWHDTPEDHALLDLLVASGLEKVNVGIEAGTAEELLLWEKRATVEDNVTIIRMLREHGIYLAMGFIPFHPYATLETIVTNAAFLRDNSGHNLRRMTERLEIYPGTAIVSRMRADGLLGESYLEGLDPYGYAFKDPRVGRLAKHFAQLYNNDDYHRHGVITEQSSVFAFETYNVVLQTFISRLHRRFTTLPGVDEVMEAFKARVHEIRQEMGRHNYGFFMSNVEAVMNDTLDPEKQRRQVVDVEHFFRDRLDVLRSEQLRVGKALTRLGARVTEVSSTIPKERPGGLPRQYTGEGSGATW

SEQ ID NO: 120 - protein, 359 aa, Streptomyces lavendulae
MPRGETGTAAARVAVCTLSSRELVGPLARLPGVAAAGTLMTANLGIEQVIKALRCDRTVRGLLVCGRDSPRFRAGQSLIALFRHGLRPEDGHIRGATGYLPVLRSVTARETEEVRARVELVDARGERDVETLRAEVAALLARVRRTPALPSREHDGGQPSFVEPDFGRLHPVGRRRSLDAGIGGFVLISVDREHRRILLRHYTSDVRPRHEMWGTRGEAMLLGLLEAGVIEDPAHAGYLGAELAKAETALRLGLHYEQDLPLRPPGRPPGPVRRRTAKERTTMAQAPALEDFLRLVTRTLGAEDAVLDLHTPLGEQLAVDSARLIELTVVLEEELGADLPDDADLARATPAELHKALVG

SEQ ID NO: 121 - protein, 145 aa, Streptomyces lavendulae
MRSVLLLNGPNLGTLGKRQPEIYGTDTLAEIEAAVAEEVGARGWEVVSEQRNGEGELVDVLQRHDDVVGAVVNPGALMIAGWSLRDALADFAPPWVEVHLSNVWGREAFRHTSVTAPLASGVVMGMGALGYRLAARALTRLVPED

SEQ ID NO: 122 - protein, 177 aa, Streptomyces lavendulae
MGRYGREGLGMSRTAEGNAGGVVVPVVRLVAVTDGPDAEGWRQALAPELVVEHGVEAIAEAAGDGGPWALVCAGAGLGAALRAAERAARPPVHVLLWLGSRGPGEGVGGEVSGQFPCPVTALVSAEVDRGRAVVPAWRGLTEGPFTVRILPAACPLPGACDQAGAQVIKEELRVWPA

SEQ ID NO: 123 - protein, 254 aa, Streptomyces lavendulae
MDATLTNDVEKASRDLVEAGYCLIECPLPAAVFEKLRGRLLEVAEQERENGSAFLYDGGNQRVFSLLNKGEEFEQNVQDPTVMLLMEEILGFGFLLSSTHANIAGPGGSRMHLHADQTFARPPWPPYPLVANSMWMLDDFTEDNGATRLVPGSHLLGRQPDYDRGEGNTETVAVCAPAGSVMVFDGRLWHQTGANTTDRPRHGILNYYCRGYVRQQQNFFSGLREDVATRATPELRRLLGYENYFSLGMTDGLP

SEQ ID NO: 124 - protein, 264 aa, Streptomyces lavendulae
MAHSPRRPDGPLRIGVWLAPQHTSVAELRAAWRAADSLGVDSLWLWDHFFPLTGDPDGSHFEAWTLLAAMAADTRAARLGTLVSNYAYRNPDLLADMARTVDHIGDGRLILGMGAGWVERDLKEYGYPTPGAGERVDGLIEAVERVDRRLGRLRPGPLGDLPLLIGGDGQRRLLRFAAERAAIWNTMAWRFAEGNRVLDEWCARVGRDPAEIERSAFVTRDQTDEELRCLVATGVQHLIFQVGHPFRFDGVERALRFAGGWSKG

SEQ ID NO: 125 - protein, 274 aa, Streptomyces lavendulae
MKISIALPNTVPGADGRLITDWARRAEERGFASLAATERLVYPGHDPLLALAAAAGATSRIGLLTNVLIGPLRTAPVLAKAVASLDSLSGGRFTLGVGPGVREDDFEAAGRAFDDRRAAFEEQLELLGRGARPGAEGPGVPVLVGGVSAAAVRRVARWADGWTAPGLEPERIVPVAERVRRAWSEAGRAGAPHVVALARYTLGEDVAQESAAFVRDYFAVLGEEAEEFVAKTPRTAGQLRAAVSALADGGVDEVVLHPTAAALSQVDRLADALL

SEQ ID NO: 126 - protein, 460 aa, Streptomyces lavendulae
MPAAGKVAVIGLDSATPQYMFDRFAEDMPVFTALRRKSLWGPMRSIDPPITMPAWSCMMSGRSPGELGVYGFRDRGAYDYGPLKFATSHSIQAPRIWDEMTAAGRSSVVLGVPGTYPPAPIRGAMVSCFLAPSTQSRYTSPPGLADELEKLTGGYALDVEDFRSTDLERVSQRVFDMSEQRFEVARHLATTQEWDFLSFVDMGPDRLHHGFWKYCDPDHPRHEPGNAYAGLFRDYYRALDRHLGRFLESLPENTTVLVVSDHGAQPMVGGLFVNEWLRKEGYLVLTEEPAGPTPVAQAAVDWKRTTAWAEGGYYGRIFLNVEGREPQGTIPAAEYESTRDLIASALEALPDDQGQPMGTRALRPGELYGEVNGIAPDLLVYVGNLRWRALATLGMGKGLYTTENDTGPDHANHGDTGIFALSAPGITPGRADGLSLYDVAPTLRELLGLAPQGSRGSLLG

SEQ ID NO: 127 - protein, 511 aa, Streptomyces lavendulae
MKAMDRVDRAVERFPMYIDGQAVQAHDGAVLRTFEPATRRHLADLPSGGAEDVRRAVSAARRAFDEGPWPRMAPGERAGLLRKAAQRLREEAEPLAELEARDNGSTLRKALGADVPGAAAAFEWSAWWAEHVPERQPEAPGSGSYVVWRPVGVVAAIVPWNLPLLLAAWRIAPAIAAGNTCVIKPASFASLSTLRLVELLHECGLPPGVVNVVTGPGGVAGEQLVRSPGVDLVAFTGSDETGAAVREGAAAAGTSARLDLGGKSPNIVLADADLDRAVTGVTWGAFLHNGQVCMAGTRAVVHADVHDDFLRLLSERVGRLRVGDPLDPATDLGPLVSRNQARTARRFTELGLSQGAELVCGGRAPAADELPPGLDAGAYFLPTVLASVGADDAVAQEEIFGPVLAVVRAGSDDDAVRIANGSRYRLSAGVWSADPARARAVAERLRADRVWINDYRLVDLELPGTAGPRSAVWDRLTNELDAYRHKHVVHGGGAGAGGVPAPPTPYALLGG

SEQ ID NO: 128 - protein, 472 aa, Streptomyces lavendulae
MKPASHSVTDTSAALGAAAAEELAAQVAGSVLLPGDEGYDEERSGFELSVEHRPALVVVATGAADVIAAVRFARARGLGIAVQATGHGKSSAATDVLISTRRMTGVRVDPRARTARIEAGVRWEQVIHEAAAHGLAPLSGSAPFVGAVSYLLGGGLGLLSRKYGFAGDHVVSLDLVTADGRFLQVSAEEHPDLFWGVRGSRGNLGIVTSVEVGLFPVTQVYGGGLFFDAGSTRAVLNTYLQWAPRMPEDMASSVFLAAYPDAEGVPGPLRGRFVTHIRLAWLGDPEEGERRFAELRAAGTVVMDTVDTLPYTRAGIIHNDPPAPVSSHSKTVMFGQLDEIAVDEILRLAGPGTDALFGVELRHLGGALARPPRHPSAVGHFPEAVFNAYVGSLVDPDTLAAVDAAQQEFVDSMRPWTTPGVCLNFLAGHNTSRETTRSAYTPEDYARLQALKSQYDPGNVFRFNPNIPPLPA

SEQ ID NO: 129 - protein, 395 aa, Streptomyces lavendulae
MTSAAPPAFPFPPGPGGTVPPEYARLLTDDPVAEVRLADGSRIWLVTRHEDVRTVLTDGRFSRHRAAMLPGSGFGRSQGSGIVDLDPPEHGRLRGPVVAAFGASRTARFAPRIEAAAEAALDRLPAGSGTVDLVAAYTAPFAGRVTAEFLGLPGDRWQDVTSDVELLLLPRGATEQALKEARGRLGQVLDELLAARRAEPGDSVTDTLLDAEELTDDDRRLLLHGLIISGFITIRDLLARHLFGVLSSPGLAARLREDPSVLPSAVQELLRYYPSSNDGLLRVATEDVVLSGRRVAAGDAVLPLVSAASRDPEVFADPHVLDIERVADRGIAFGAGQHACPATGLAVTELTVGIGRLLAAFPRIALAVPPEEVEHSSELLPLGVRSLPVVPGPRN

SEQ ID NO: 130 - protein, 474 aa, Streptomyces lavendulae
MLPEFQLQWNWLDAPAGGGGELQATWARLRIAVGAETVTLVQEPGQGTFREHTTGSLYPLAEWIAFNWWSLVADARPGTQISQLRFAYRHGVGDNRGSWWMRSRRHILRAACDGFRWPDMLFVPEGRETRIVWMPDMGPDVRPGNRFASRGNSCVESAAFTATLASFVDAVTERLTDQGITGTPLQEEWAAVRATDEDEAAFCRIAARLGLDPYAEAEPYEADILKAAEQLAEPLASDFFNGVRPERIADQLQWIARVRTLMGTAPADTPLPPALVELRKDCADLSEKFFAPGRLDNPWDLGYEVAHRVRAWAGLDDTAPFDPAPLMGYRTEQVPYMDRGLVALGTRRGADGPVLVSSRRFTDRPRRFLQARALWHLICDPDDTFLIAAAHTHRQHVARGFALEVLAPAKGVATLLADPGHLVSAEDVEVIADDYGCGNIVVEHQLDNRVLAKDFTWPGHARAGAPAGERSRGA

SEQ ID NO: 131 - protein, 443 aa, Streptomyces lavendulae
MTIRQRVVVVITEGAAPELLDRWCAQGLLPGFAALRSQGASGPLHAEGTPYEPPGLLSVLTGRRAADHGFYSYWTCHDPEYAPQVLTPEHRRHPLLWQHEVFQGVRFASIGLFGTHPPEPFDGSLITYPMYATLHACHPRSLQRTLAKKGIRPVHDVSIFWTGQDRDELLPSLLEADVQRGRAALALLEESDVVIVNLTSIDRCSHIYWQELEHGPEHERESAVFAAYRTCDQVIQDALRAADDRTSVVAFSEIGFGPLRNYCSINDEMEQAGFLATAEDGRVEWAGSAAFEAVQGTHGVNINLRDRYKHGLVPERDYEKVRTDVAAALLERRNPRTGRLFFDAVRRREEVYPGEATQHAPDLILEPADWRYLPLGDPHWASHVHRDWQSGWHRRESYWSAVGPGFTGGARQTRTAAPVDIPATVCALLGRDVPNDWDGVPLS

SEQ ID NO: 132 - protein, 123 aa, Streptomyces lavendulae
MTPEELSDFALELPEAVDDEAFGPGAAVFKVEKKVFAILQDASEDRPPQVTLKCEPDLALHLREQYAAVVPGYHVNKRHWNTVVLNGTVPVEELREMVEHSYDRVVAGLPKAVRERLRLLRTV

SEQ ID NO: 133 - protein, 351 aa, Streptomyces lavendulae
MTVEQTPENPGTAARAAAEETVNDILQGAWKARAIHVAVELGVPELLQEGPRTATALAEATGAHEQTLRRLLRLLATVGVFDDLGHDDLFAQNALSAVLLPDPASPVATDARFQAAPWHWRAWEQLTHSVRTGEASFPSTWPTAPRSGSSPTRDPKARELFNRAMGSVSLTEAGQVAAAYDFSGAATAVDIGGGRGSLMAAVLDAFPGLRGTLLERPPVAEEARELLTGRGLADRCEILPGDFFETIPDGADVYLIKHVLHDWDDDDVVRILRRIATAMKPDSRLLVIDNLIDERPAASTLFVDLLLLVLVGGAERSESEFAALLEKSGLRVERSLPCGAGPVRIVEIRRA

SEQ ID NO: 134 - protein, 546 aa, Streptomyces lavendulae
MTVLGLGGSGHDWASCATDGRRLVAIDEERLVRSKYGLGADLLAGHSRRAVLDALGTSAEAVEHVVACELVPRPFYHSFRRRVTVVNHHLAHAYSAFGASGMTRAAVLVCDNSGSLVTGLKSGPGPREAETISCYTADASGLRLVNRVAGTHAVDASSESAYYQPGETDNSLGHFYRSASLALGLAYSGPKTRYPVSEDGKTMGLAPYGDDRFVDEVAELVTLLPEGGVQISASKVNHLFERLVESGEFEDRAALAYAAQETLERALLHCARDLHRRTGLTDLCIAGGVGLNSVANGRILRETPFERVFVVPAAGDNGISLGCAYYGLHELEGRAPSELPALDTAYLGPDYPAERVDAALAGSGFTVETPDDLPGRVAGLLAEGKIIGWFDGRSEFGPRALGHRSILAAPFPASVRDHLNDNVKHREWFRPYAPIVREDRAADYFDLVQPSPFMLVVARVTRQDAIPAATHVDGTARLQTLNAAQNPKVYELLGRFEALTGCAVLLNTSFNVAGQPIVETPEDAVEAFAGMRLDHLVVGDRLATKP

SEQ ID NO: 135 - protein, 568 aa, Streptomyces lavendulae
MDVPVLVVGGGPTGLAMALFLARHGVGCLLVERRTTTSPVPRATHVSRRSMELFREAGLEEEIRRAGFEVVREDDPRLRTRPERHLPRVVLQAASLAGPGPVGVLETGDEELAVPGPCAPFWCGQDRMEPLLAKAAARHGADVRFGHELTGLWPGEDSTRARVRAAGTGRTYTVDARFVIAADGARGEIAERVGIAREGLGTVAHRVSILFRADPGRWARDRRFFMCMIQNPGFDGAVMELNTPGRWCAAVDYDPARAEPDGTYSARTCLDLVRAAVGDDRSDAAVDTVFHWKARHRIAAAYRSGAVFLIGDAAHLHPPSGGYGSNVGFQDAHNLAWKIAAVLGGWAGPRLLDTYDEERRPVGKATAEQSMLLDGVPPEPLGGSVVRCDPRTLIMGYRYHSAAVLGPPHGPAFPAAFTLRGDPGTRLPHVWLRTDAGERVSTLDLCHGHFVLLSADPVWAAAAARSAKETGVPLRGHHLAATGSELADPSGEFPRSCGTGPAGAVLVRPDGMVAWRTARAVPPDPDSAQDLVTAAVRRVLALPERAAPPVLGPPRLSRGSYRRVGSDG

SEQ ID NO: 136 - protein, 160 aa, Streptomyces lavendulae
MKPHSFCTCWPGATVWLTGPPGAGKTTIARALAERLRERGRRVEVLDGDATRALLTAGSSWEDRGTGLQRVGLMAEVLARNGIVVLVPVTAARADSREAVRRRHERSGTAHLEVRVVRDAVPPSGLPAPPGPDLRIAAHEQSAEESARALHRLLAERELA

SEQ ID NO: 137 - protein, 319 aa, Streptomyces lavendulae
MNPGRGGAYAAGRDGTRGTRRPHGLSHLDLLESESVHIFREVAGEFERPVILFSGGKDSIVMLHLALKSFAPAPVPFALLHVDTGHNFPEVIAYRDRVVAALGLRLEVASVQDFIDNGTLRERPDGTRNPLQTVPLLDAIGRHRFDAVFGGGRRDEEKARAKERVFSLRDEFGGWDPRRQRPELWRLYNGRHAPGEHVRVFPLSNWTELDVWQYVAREEIELPTIYYAHEREVFRRGGMWLAPGEWGGPREGEAVEKRRVRYRTVGDMSCTGAVDSAAATVADVVAEIATSRLTERGATRADDKLSEAAMEDRKREGYF

SEQ ID NO: 138 - protein, 163 aa, Streptomyces lavendulae
MGQDSRPRWLTDEEQRVWRGYLRATRLVEDHLDRRLQREADMPHLYYGLLVQLSEAPRRGIRMTDLARNAKITRPRLSHAITRLEKLGWVRRESCHGDRRGQNAVLTEEGREVLEKSAPGHVAAVRAAVFDSLTPEQVGQLGRICQAIEKGLDREGADLPWLR

SEQ ID NO: 139 - protein, 413 aa, Streptomyces lavendulae
MERHDGAPGWGFTHTQYSADHGERGATRRAGALLSARPLPQNQHIMGWGAENPEPAPGRYDFEVLDERVALMRATGATPVLTLCAAPDWMKGGRPGRTDWSRLETAPDPRHYADFARLAGVIAQRYPDIRHFLVWNELKGFYDEDRRRWDYEGYTRLYNLVHAELKRRNPRNLVGGPYAVVDHDPPAEDAADRSRELRGPWGELDQRSADVIRYWNAHKAGADFVVVDGSSYTREGHRAIPDEFAATEKFADVTRWVRSVTGLPVWWAEWYVEPPAEDDRPGGRDGWGEGHRTAVQATAMMRLAESGASAAFYWNPQRTGKACPGCLWRSTHLRDGGGELPMAGLLSRFAREFPPGTAFRPVAVTCGSGDRVEALADEAAVLVVNTECRPVAARVDGQALSLAPYEVRWLTRP

SEQ ID NO: 140 - protein, 270 aa, Streptomyces lavendulae
MEFLGPAAGVSGATRLYAVLGDPVAQVKAPGLLNPLLSESGLDAVVVPVHVRARDLAEVVEGLKRIGNLDGLLVTVPHKAALCGLADGLGPAAALIGTANAMRREPDGRWYAENFDGLGFVQGLQAAGHTVRDRHVALVGAGGAGSAIATALLMADAARVSVHDTDRAQLDALLLRLGSRRPDGIRALGPGDLEAADFAVNATPLGMRSEDPLPFDPARVRPDAVVVDVVMKPHETALLSAAATAGRRVHHGIHMLEQQVPCYRAFFGWP

SEQ ID NO: 141 - protein, 271 aa, Streptomyces lavendulae
MTRRRPTGPIHRRRASLTLSPTGAAMRRNRIAALLPAALALVGISVLAPATTASAAAPHGGTSQAAAFPVSEAQFKQMFPKRNAFYTYKGLVAALKAYPGFAGTGSAEVRKQEAAAFLANVAHETGGLVYVVEQNTANYPHYCDRSRPYGCPAGQAAYYGRGPLQISWNFNYKAAGDALGIDLLHNPSLVQKDAAVSWKTGLWYWNTQRGPGTMTPHEAMVNHRGFGQTIRSINGALECDGHNPAQVQSRVANYQRFTKILGVAPGGNLSC

SEQ ID NO: 142 - protein, 391 aa, Artificial Sequence
  A consensus sequence
MXXXXXXXXXXFPXWPQXDDAERXGLXRALXQGQWWRXGGXEVXXFEREFAXXHGAXHALAVTNGTHALELALXVMGVGPGTEVIVPAFTFISSSQAXQRLGAVXVPVDVDPXTYCLDXXAAAXAVTPRTXAIMPVHMAGQXADMDALXKXSAXTGVPXXQDAAHAHGAXWXGXRVGELGSIAXFSFQNGKLMTAGEGGAVLFPDXEXXXXEXAFLXHSCGRPXXDRXYFHXTXGSNXRXNEFSASVLRAQLXRLDXQIXXRXERWXXLSXLLAXIDGVVPQXXDXRXDRNXHYMAMFRXPGXTEERRXAXVDXLVERGXPAFXAFRXVYRTXAFWEXGAPDXXXXELAXRCPXXXXIXXDCXWLHHRVLLXXEXXXXXXAXVXADXVXXX

SEQ ID NO: 143 - DNA, 393 bp, Streptomyces lavendulae
atgtcagcaaggatttccctcttcgccgtggtggtcgaggacatggccaagtcgctggagttctaccggaagctgggcgtcgagatccccgccgaggccgactccgcgccgcacacggaggccgtgctcgacggcggcatccggctcgcctgggacaccgtggagacggtgcgcagctacgaccccgagtggcaggcccccaccggcggccaccgcttcgccatcgcgttcgagttccccgacaccgcgagcgtggacaagaagtacgccgagctcgtcgacgccggctacgagggccacctcaagccgtggaacgccgtgtggggtcagcgctacgccatcgtcaaggaccccgacggcaacgtggtggacctcttcgcgcccctcccgtaa

SEQ ID NO: 144 - protein, 16 aa, Artificial Sequence
  A motif
VXGXLXDXXGRKXXXL

SEQ ID NO: 145 - protein, 11 aa, Artificial Sequence
  A motif
LDXTVXNVALP

SEQ ID NO: 146 - DNA, 23 bp, Artificial Sequence
  A primer
gctctagatctacgtctcccgcg

SEQ ID NO: 147 - DNA, 26 bp, Artificial Sequence
  A primer
gcctcgagcatggacgatccctctcg

SEQ ID NO: 148 - DNA, 26 bp, Artificial Sequence
  A primer
gcctcgagccgggaaagtgagcggca

SEQ ID NO: 149 - DNA, 23 bp, Artificial Sequence
  A primer
gcaagcttcggcatcacgcgcca

SEQ ID NO: 150 - DNA, 26 bp, Artificial Sequence
  A primer
gcctcgagcgtcatgccgctcacttt

SEQ ID NO: 151 - DNA, 26 bp, Artificial Sequence
  A primer
gcctcgagtagggctcccacgggaag

SEQ ID NO: 152 - DNA, 30 bp, Artificial Sequence
  A primer
gggatcgcatatgccgcactccgagctgtc

SEQ ID NO: 153 - DNA, 29 bp, Artificial Sequence
  A primer
gtgagccatatgacggaaaccgcgtccgc